(19) United States
(12) Patent Application Publication
(10) Pub. No.: US 2025/0333515 A1
(43) Pub. Date: Oct. 30, 2025

(54) BINDING AGENTS AND METHODS OF USE THEREOF

(71) Applicant: VOR BIOPHARMA INC., Cambridge, MA (US)

(72) Inventors: Julian Scherer, Cambridge, MA (US); Nawazi Fazlullah Salar Khan, Cambridge, MA (US); Mariana Araujo Alves Martins Da Silva, Cambridge, MA (US); Tirtha Chakraborty, Cambridge, MA (US)

(21) Appl. No.: 18/855,405

(22) PCT Filed: Apr. 11, 2023
(86) PCT No.: PCT/US2023/065642

§ 371 Date: Oct. 9, 2024

Related U.S. Application Data
(60) Provisional application No. 63/329,664, filed on Apr. 11, 2022.
(60) Provisional application No. 63/399,588, filed on Aug. 19, 2022.

(51) Int. Cl. C07K 16/28; A61K 35/17; A61K 40/15; A61K 40/31; A61K 45/06; A61P 35/02; C07K 14/705; C07K 14/725; C12N 5/0783; C12N 9/22

(52) U.S. Cl. CPC C07K 16/2851; A61K 35/17; A61K 40/15; A61K 40/31; A61K 45/06; A61P 35/02; C07K 14/7051; C07K 14/70521; C12N 5/0646; C12N 9/226; A61K 2239/13; A61K 2239/21; A61K 2239/22; C07K 2317/24; C07K 2317/524; C07K 2317/526; C07K 2317/565; C07K 2317/569; C12N 2310/20; C12N 2510/00

(57) ABSTRACT

The present disclosure includes agents that specifically bind CLL-1, such as antibodies and chimeric antigen receptors, as well as methods of making and using such agents.

### RELATED APPLICATIONS

This application claims the benefit under 35 U.S.C. 119(e) of U.S. provisional application No. 63/329,664 filed Apr. 11, 2022 and U.S. provisional application No. 63/399,588, filed Aug. 19, 2022, which are incorporated by reference herein in their entirety.

### REFERENCE TO AN ELECTRONIC SEQUENCE LISTING

The contents of the electronic sequence listing (V029170034WO00-SEQ-CEW.xml; Size: 875,826 bytes; and Date of Creation: Apr. 11, 2023) is herein incorporated by reference in its entirety.

### BACKGROUND

C-type lectin-like molecule-1 (CLL-1), which may also be referred to as CLEC12A, is a C-type lectin-like receptor family member that is frequently expressed on acute myeloid leukemia (AML) cells. AML remains a major therapeutic challenge and an unmet need in hematologic oncology. AML is a disease resulting in uncontrollable accumulation of immature myeloid blasts in the bone marrow and peripheral blood, and the disease has multiple subtypes that contribute to the challenge in developing an encompassing targeted therapy. Although there is an increased understanding in the molecular genetics of the disease, there have been relatively few novel therapies approved for AML. Accordingly, there remains a need for novel therapeutics for AML.

### SUMMARY

In one aspect, the disclosure is directed to an anti-CLL-1 antibody, or antigen-binding fragment thereof, comprising an amino acid sequence of SEQ ID NOs: 15, 29, 43, 57, 71, 85, 99, 113, 127, 141, 155, 169, 174, 179, 184, 189, 194, 199, 204, 209, 214, 219, 224, 229, 234, 239, 244, 249, 254, 259, 264, 269, 275, 280, 285, 290, 295, 309, 323, 337, 351, 365, 418, 422, 426, 430, 434, 438, 442, 446, 450, 454, 458, 462, 466, 470, 474, 478, 482, 486, 490, 494, 498, 502, 506, 510, 514, 518, 522, 526, 530, 534, 538, 542, 546, 550, 554, 558, 562, 566, 570, 574, 578, 582, 586, 590, 594, 598, 602, 606, 610, 614, 618, 622, 626, 630, 634, 638, 642, 646, 650, 654, 658, 662, 666, 670, 674, 678, 682, 686, 690, 694, 698, 702, 706, 710, 714, 718, 722, 726, 730, 734, 738, 742, 746, 750, 754, 758, 762, 766, 774, 779, 784, 788, 793, 798, 801, 804, 808, 819, 831, 837, 849, 900, 905, 910, 918, or 920.

In one aspect, the disclosure is directed to an anti-CLL-1 antibody, or antigen-binding fragment thereof, comprising at least one complementarity determining region (CDR) sequence of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 820-825, 838-843, 850-854, 861-866, 896-898, 901-903, 906-908, or 911-916.

In some embodiments, the anti-CLL-1 antibody, or antigen-binding fragment thereof, comprises three CDR sequences of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 820-825, 838-843, 850-854, 861-866, 896-898, 901-903, 906-908, or 911-916.

In some embodiments, the anti-CLL-1 antibody, or antigen-binding fragment thereof, comprises six CDR sequences of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 820-825, 838-843, 850-854, 861-866, 896-898, 901-903, 906-908, or 911-916.

In some embodiments, the anti-CLL-1 antibody, or antigen-binding fragment thereof, comprises three heavy chain CDR sequences of SEQ ID NOs: 5-7, 19-21, 33-35, 47-49, 61-63, 75-77, 89-91, 103-105, 117-119, 131-133, 145-147, 159-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 299-301, 313-315, 327-329, 341-343, 355-357, 423-425, 431-433, 439-441, 447-449, 455-457, 463-465, 471-473, 479-481, 487-489, 495-497, 503-505, 511-513, 519-521, 527-529, 535-537, 543-545, 551-553, 559-561, 567-569, 575-577, 583-585, 591-593, 599-601, 607-609, 615-617, 623-625, 631-633, 639-641, 647-649, 655-657, 663-665, 671-673, 679-681, 687-689, 695-697, 703-705, 711-713, 715-746, 748-808, 816-817, 823-825, 828-829, 834-835, 841-843, 846-847, 852-854, 857-858, 869-870, 880-881, 892-893, 896-910, 914-916, or 919-920.

In another aspect, the disclosure is directed to an anti-CLL-1 antibody, or antigen-binding fragment thereof, comprising a CDR1, CDR2, and CDR3 of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 820-825, 838-843, 850-854, 896-898, 901-903, 906-908, or 911-916.

In another aspect, the disclosure is directed to an anti-CLL-1 antibody, or antigen-binding fragment thereof, comprising at least one CDR that is at least 85%, 90%, 91%, 92%, 93%, 94%, 95%, 96%, 97%, 98%, 99%, or 100% identical to a CDR (e.g., CDR1, CDR2, and/or CDR3) of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 820-825, 838-843, 850-854, 896-898, 901-903, 906-908, or 911-916.

In another aspect, the disclosure is directed to an anti-CLL-1 antibody, or antigen-binding fragment thereof, comprising a heavy chain variable region comprising a CDR1 provided by SEQ ID NO: 5, 19, 33, 47, 61, 75, 89, 103, 117, 131, 145, 159, 170, 175, 180, 185, 190, 195, 200, 205, 210, 215, 220, 225, 230, 235, 240, 245, 250, 255, 260, 265, 271, 276, 281, 286, 291, 299, 313, 327, 341, 355, 423, 431, 439, 447, 455, 463, 471, 479, 487, 495, 503, 511, 519, 527, 535, 543, 551, 559, 567, 575, 583, 591, 599, 607, 615, 623, 631, 639, 647, 655, 663, 671, 679, 687, 695, 703, 711, 715, 719, 723, 727, 731, 735, 739, 743, 747, 751, 755, 759, 763, 767, 770, 775, 780, 785, 789, 794, 823, 841, 852, 896, 901, 906, or 914; a CDR2 provided by SEQ ID NO: 6, 20, 34, 48, 62, 76, 90, 104, 118, 132, 146, 160, 171, 176, 181, 186, 191, 196, 201, 206, 211, 216, 221, 226, 231, 236, 241, 246, 251, 256, 261, 266, 272, 277, 282, 287, 292, 300, 314, 328, 342 356, 424, 432, 440, 448, 456, 464, 472, 480, 488, 496, 504, 512, 520, 528, 536, 544, 552, 560, 568, 576, 584, 592, 600, 608, 616, 624, 632, 640, 648, 656, 664, 672, 680, 688, 696, 704, 712, 716, 720, 724, 728, 732, 736, 740, 744, 748, 752, 756, 760, 764, 768, 771, 776, 781, 786, 790, 795, 799, 802, 805, 824, 842, 853, 897, 902, 907, or 915; and a CDR3 provided by SEQ ID NO: 7, 21, 35, 49, 63, 77, 91, 105, 119, 133, 147, 161, 172, 177, 182, 187, 192, 197, 202, 207, 212, 217, 222, 227, 232, 237, 242, 247, 252, 257, 262, 267, 273, 278, 283, 288, 293, 301, 315, 329, 343, 357, 425, 433, 441, 449, 457, 465, 473, 481, 489, 497, 505, 513, 521, 529, 537, 545, 553, 561, 569, 577, 585, 593, 601, 609, 617, 625, 633, 641, 649, 657, 665, 673, 681, 689, 697, 705, 713, 717, 721, 725, 729, 733, 737, 741, 745, 749, 753, 757, 761, 765, 769, 772, 777, 782, 791, 796, 806, 825, 843, 854, 898, 903, 908, or 916; and a light chain variable region comprising a CDR1 provided by SEQ ID NO: 2, 16, 30, 44, 58, 72, 86, 100, 114, 128, 142, 156, 296, 310, 324, 338, 352, 419, 427, 435, 443, 451, 459, 467, 475, 483, 491, 499, 507, 515, 523, 531, 539, 547, 555, 563, 571, 579, 587, 595, 603, 611, 619, 627, 635, 643, 651, 659, 667, 675, 683, 691, 699, 707, 820, 838, 850, or 911; a CDR2 provided by SEQ ID NO: 3, 17, 31, 45, 59, 73, 87, 101, 115, 129, 143, 157, 297, 311, 325, 339, 353, 420, 428, 436, 444, 452, 460, 468, 476, 484, 492, 500, 508, 516, 524, 532, 540, 548, 556, 564, 572, 580, 588, 596, 604, 612, 620, 628, 636, 644, 652, 660, 668, 676, 684, 692, 700, 708, 821, 839, or 91; and a CDR3 provided by SEQ ID NO: 4, 18, 32, 46, 60, 74, 88, 102, 116, 130, 144, 158, 298, 312, 326, 340, 354, 421, 429, 437, 445, 453, 461, 469, 477, 485, 493, 501, 509, 517, 525, 533, 541, 549, 557, 565, 573, 581, 589, 597, 605, 613, 621, 629, 637, 645, 653, 661, 669, 677, 685, 693, 701, 709, 822, 840, 851, or 913.

In some embodiments, the anti-CLL-1 antibody, or antigen-binding fragment thereof, comprises: a heavy chain variable region comprising a CDR1 provided by SEQ ID NO: 5, a CDR2 provided by SEQ ID NO: 6, and a CDR3 provided by SEQ ID NO: 7; and a light chain variable region comprising a CDR1 provided by SEQ ID NO: 2, a CDR2 provided by SEQ ID NO: 3, and a CDR3 provided by SEQ ID NO: 4.

In some embodiments, the anti-CLL-1 antibody, or antigen-binding fragment thereof, comprises: a heavy chain variable region comprising a CDR1 provided by SEQ ID NO: 19, a CDR2 provided by SEQ ID NO: 20, and a CDR3 provided by SEQ ID NO: 21; and a light chain variable region comprising a CDR1 provided by SEQ ID NO: 16, a CDR2 provided by SEQ ID NO: 17, and a CDR3 provided by SEQ ID NO: 18.

In some embodiments, the anti-CLL-1 antibody, or antigen-binding fragment thereof, comprises: a heavy chain variable region comprising a CDR1 provided by SEQ ID NO: 33, a CDR2 provided by SEQ ID NO: 34, and a CDR3 provided by SEQ ID NO: 35; and a light chain variable region comprising a CDR1 provided by SEQ ID NO: 30, a CDR2 provided by SEQ ID NO: 31, and a CDR3 provided by SEQ ID NO: 32.

In some embodiments, the anti-CLL-1 antibody, or antigen-binding fragment thereof, comprises: a heavy chain variable region comprising a CDR1 provided by SEQ ID NO: 47, a CDR2 provided by SEQ ID NO: 48, and a CDR3 provided by SEQ ID NO: 49; and a light chain variable region comprising a CDR1 provided by SEQ ID NO: 44, a CDR2 provided by SEQ ID NO: 45, and a CDR3 provided by SEQ ID NO: 46.

In some embodiments, the anti-CLL-1 antibody, or antigen-binding fragment thereof, comprises: a heavy chain variable region comprising a CDR1 provided by SEQ ID NO: 61, a CDR2 provided by SEQ ID NO: 62, and a CDR3 provided by SEQ ID NO: 63; and a light chain variable region comprising a CDR1 provided by SEQ ID NO: 58, a CDR2 provided by SEQ ID NO: 59, and a CDR3 provided by SEQ ID NO: 60.

In some embodiments, the anti-CLL-1 antibody, or antigen-binding fragment thereof, comprises: a heavy chain variable region comprising a CDR1 provided by SEQ ID NO: 75, a CDR2 provided by SEQ ID NO: 76, and a CDR3 provided by SEQ ID NO: 77; and a light chain variable region comprising a CDR1 provided by SEQ ID NO: 72, a CDR2 provided by SEQ ID NO: 73, and a CDR3 provided by SEQ ID NO: 74.

In some embodiments, the anti-CLL-1 antibody, or antigen-binding fragment thereof, comprises: a heavy chain variable region comprising a CDR1 provided by SEQ ID NO: 89, a CDR2 provided by SEQ ID NO: 90, and a CDR3 provided by SEQ ID NO: 91; and a light chain variable region comprising a CDR1 provided by SEQ ID NO: 86, a CDR2 provided by SEQ ID NO: 87, and a CDR3 provided by SEQ ID NO: 88.

In some embodiments, the anti-CLL-1 antibody, or antigen-binding fragment thereof, comprises: a heavy chain variable region comprising a CDR1 provided by SEQ ID NO: 103, a CDR2 provided by SEQ ID NO: 104, and a CDR3 provided by SEQ ID NO: 105; and a light chain variable region comprising a CDR1 provided by SEQ ID NO: 100, a CDR2 provided by SEQ ID NO: 101, and a CDR3 provided by SEQ ID NO: 102.

In some embodiments, the anti-CLL-1 antibody, or antigen-binding fragment thereof, comprises: a heavy chain variable region comprising a CDR1 provided by SEQ ID NO: 117, a CDR2 provided by SEQ ID NO: 118, and a CDR3 provided by SEQ ID NO: 119; and a light chain variable region comprising a CDR1 provided by SEQ ID NO: 114, a CDR2 provided by SEQ ID NO: 115, and a CDR3 provided by SEQ ID NO: 116.

In some embodiments, the anti-CLL-1 antibody, or antigen-binding fragment thereof, comprises: a heavy chain variable region comprising a CDR1 provided by SEQ ID NO: 131, a CDR2 provided by SEQ ID NO: 132, and a CDR3 provided by SEQ ID NO: 133; and a light chain variable region comprising a CDR1 provided by SEQ ID NO: 128, a CDR2 provided by SEQ ID NO: 129, and a CDR3 provided by SEQ ID NO: 130.

In some embodiments, the anti-CLL-1 antibody, or antigen-binding fragment thereof, comprises: a heavy chain variable region comprising a CDR1 provided by SEQ ID NO: 145, a CDR2 provided by SEQ ID NO: 146, and a CDR3 provided by SEQ ID NO: 147; and a light chain variable region comprising a CDR1 provided by SEQ ID NO: 142, a CDR2 provided by SEQ ID NO: 143, and a CDR3 provided by SEQ ID NO: 144.

In some embodiments, the anti-CLL-1 antibody, or antigen-binding fragment thereof, comprises: a heavy chain variable region comprising a CDR1 provided by SEQ ID NO: 159, a CDR2 provided by SEQ ID NO: 160, and a CDR3 provided by SEQ ID NO: 161; and a light chain variable region comprising a CDR1 provided by SEQ ID NO: 156, a CDR2 provided by SEQ ID NO: 157, and a CDR3 provided by SEQ ID NO: 158.

In some embodiments, the anti-CLL-1 antibody, or antigen-binding fragment thereof, comprises: a heavy chain variable region comprising a CDR1 provided by SEQ ID NO: 299, a CDR2 provided by SEQ ID NO: 300, and a CDR3 provided by SEQ ID NO: 301; and a light chain variable region comprising a CDR1 provided by SEQ ID NO: 296, a CDR2 provided by SEQ ID NO: 297, and a CDR3 provided by SEQ ID NO: 298.

In some embodiments, the anti-CLL-1 antibody, or antigen-binding fragment thereof, comprises: a heavy chain variable region comprising a CDR1 provided by SEQ ID NO: 313, a CDR2 provided by SEQ ID NO: 314, and a CDR3 provided by SEQ ID NO: 315; and a light chain variable region comprising a CDR1 provided by SEQ ID NO: 310, a CDR2 provided by SEQ ID NO: 311, and a CDR3 provided by SEQ ID NO: 312.

In some embodiments, the anti-CLL-1 antibody, or antigen-binding fragment thereof, comprises: a heavy chain variable region comprising a CDR1 provided by SEQ ID NO: 327, a CDR2 provided by SEQ ID NO: 328, and a CDR3 provided by SEQ ID NO: 329; and a light chain variable region comprising a CDR1 provided by SEQ ID NO: 324, a CDR2 provided by SEQ ID NO: 325, and a CDR3 provided by SEQ ID NO: 326.

In some embodiments, the anti-CLL-1 antibody, or antigen-binding fragment thereof, comprises: a heavy chain variable region comprising a CDR1 provided by SEQ ID NO: 341, a CDR2 provided by SEQ ID NO: 342, and a CDR3 provided by SEQ ID NO: 343; and a light chain variable region comprising a CDR1 provided by SEQ ID NO: 338, a CDR2 provided by SEQ ID NO: 339, and a CDR3 provided by SEQ ID NO: 340.

In some embodiments, the anti-CLL-1 antibody, or antigen-binding fragment thereof, comprises: a heavy chain variable region comprising a CDR1 provided by SEQ ID NO: 355, a CDR2 provided by SEQ ID NO: 356, and a CDR3 provided by SEQ ID NO: 357; and a light chain variable region comprising a CDR1 provided by SEQ ID NO: 352, a CDR2 provided by SEQ ID NO: 353, and a CDR3 provided by SEQ ID NO: 354.

In some embodiments, the anti-CLL-1 antibody, or antigen-binding fragment thereof, comprises: a heavy chain variable region comprising a CDR1 provided by SEQ ID NO: 423, a CDR2 provided by SEQ ID NO: 424, and a CDR3 provided by SEQ ID NO: 425; and a light chain variable region comprising a CDR1 provided by SEQ ID NO: 418, a CDR2 provided by SEQ ID NO: 419, and a CDR3 provided by SEQ ID NO: 421.

In some embodiments, the anti-CLL-1 antibody, or antigen-binding fragment thereof, comprises: a heavy chain variable region comprising a CDR1 provided by SEQ ID NO: 431, a CDR2 provided by SEQ ID NO: 432, and a CDR3 provided by SEQ ID NO: 433; and a light chain variable region comprising a CDR1 provided by SEQ ID NO: 427, a CDR2 provided by SEQ ID NO: 428, and a CDR3 provided by SEQ ID NO: 429.

In some embodiments, the anti-CLL-1 antibody, or antigen-binding fragment thereof, comprises: a heavy chain variable region comprising a CDR1 provided by SEQ ID NO: 439, a CDR2 provided by SEQ ID NO: 440, and a CDR3 provided by SEQ ID NO: 441; and a light chain variable region comprising a CDR1 provided by SEQ ID NO: 435, a CDR2 provided by SEQ ID NO: 436, and a CDR3 provided by SEQ ID NO: 437.

In some embodiments, the anti-CLL-1 antibody, or antigen-binding fragment thereof, comprises: a heavy chain variable region comprising a CDR1 provided by SEQ ID NO: 447, a CDR2 provided by SEQ ID NO: 448, and a CDR3 provided by SEQ ID NO: 449; and a light chain variable region comprising a CDR1 provided by SEQ ID NO: 443, a CDR2 provided by SEQ ID NO: 444, and a CDR3 provided by SEQ ID NO: 445.

In some embodiments, the anti-CLL-1 antibody, or antigen-binding fragment thereof, comprises: a heavy chain variable region comprising a CDR1 provided by SEQ ID NO: 455, a CDR2 provided by SEQ ID NO: 456, and a CDR3 provided by SEQ ID NO: 457; and a light chain variable region comprising a CDR1 provided by SEQ ID NO: 451, a CDR2 provided by SEQ ID NO: 452, and a CDR3 provided by SEQ ID NO: 453.

In some embodiments, the anti-CLL-1 antibody, or antigen-binding fragment thereof, comprises: a heavy chain variable region comprising a CDR1 provided by SEQ ID NO: 463, a CDR2 provided by SEQ ID NO: 464, and a CDR3 provided by SEQ ID NO: 465; and a light chain variable region comprising a CDR1 provided by SEQ ID NO: 459, a CDR2 provided by SEQ ID NO: 460, and a CDR3 provided by SEQ ID NO: 461.

In some embodiments, the anti-CLL-1 antibody, or antigen-binding fragment thereof, comprises: a heavy chain variable region comprising a CDR1 provided by SEQ ID NO: 471, a CDR2 provided by SEQ ID NO: 472, and a CDR3 provided by SEQ ID NO: 473; and a light chain variable region comprising a CDR1 provided by SEQ ID NO: 467, a CDR2 provided by SEQ ID NO: 468, and a CDR3 provided by SEQ ID NO: 469.

In some embodiments, the anti-CLL-1 antibody, or antigen-binding fragment thereof, comprises: a heavy chain variable region comprising a CDR1 provided by SEQ ID NO: 479, a CDR2 provided by SEQ ID NO: 480, and a CDR3 provided by SEQ ID NO: 481; and a light chain variable region comprising a CDR1 provided by SEQ ID NO: 479, a CDR2 provided by SEQ ID NO: 480, and a CDR3 provided by SEQ ID NO: 481.

In some embodiments, the anti-CLL-1 antibody, or antigen-binding fragment thereof, comprises: a heavy chain variable region comprising a CDR1 provided by SEQ ID NO: 487, a CDR2 provided by SEQ ID NO: 488, and a CDR3 provided by SEQ ID NO: 489; and a light chain variable region comprising a CDR1 provided by SEQ ID NO: 483, a CDR2 provided by SEQ ID NO: 484, and a CDR3 provided by SEQ ID NO: 485.

In some embodiments, the anti-CLL-1 antibody, or antigen-binding fragment thereof, comprises: a heavy chain variable region comprising a CDR1 provided by SEQ ID NO: 495, a CDR2 provided by SEQ ID NO: 496, and a CDR3 provided by SEQ ID NO: 497; and a light chain variable region comprising a CDR1 provided by SEQ ID NO: 491, a CDR2 provided by SEQ ID NO: 492, and a CDR3 provided by SEQ ID NO: 493.

In some embodiments, the anti-CLL-1 antibody, or antigen-binding fragment thereof, comprises: a heavy chain variable region comprising a CDR1 provided by SEQ ID NO: 503, a CDR2 provided by SEQ ID NO: 504, and a CDR3 provided by SEQ ID NO: 505; and a light chain variable region comprising a CDR1 provided by SEQ ID NO: 499, a CDR2 provided by SEQ ID NO: 500, and a CDR3 provided by SEQ ID NO: 501.

In some embodiments, the anti-CLL-1 antibody, or antigen-binding fragment thereof, comprises: a heavy chain variable region comprising a CDR1 provided by SEQ ID NO: 511, a CDR2 provided by SEQ ID NO: 512, and a CDR3 provided by SEQ ID NO: 513; and a light chain variable region comprising a CDR1 provided by SEQ ID NO: 507, a CDR2 provided by SEQ ID NO: 508, and a CDR3 provided by SEQ ID NO: 509.

In some embodiments, the anti-CLL-1 antibody, or antigen-binding fragment thereof, comprises: a heavy chain variable region comprising a CDR1 provided by SEQ ID NO: 519, a CDR2 provided by SEQ ID NO: 520, and a CDR3 provided by SEQ ID NO: 521; and a light chain variable region comprising a CDR1 provided by SEQ ID NO: 515, a CDR2 provided by SEQ ID NO: 516, and a CDR3 provided by SEQ ID NO: 517.

In some embodiments, the anti-CLL-1 antibody, or antigen-binding fragment thereof, comprises: a heavy chain variable region comprising a CDR1 provided by SEQ ID NO: 527, a CDR2 provided by SEQ ID NO: 528, and a CDR3 provided by SEQ ID NO: 529; and a light chain variable region comprising a CDR1 provided by SEQ ID NO: 523, a CDR2 provided by SEQ ID NO: 524, and a CDR3 provided by SEQ ID NO: 525.

In some embodiments, the anti-CLL-1 antibody, or antigen-binding fragment thereof, comprises: a heavy chain variable region comprising a CDR1 provided by SEQ ID NO: 535, a CDR2 provided by SEQ ID NO: 536, and a CDR3 provided by SEQ ID NO: 537; and a light chain variable region comprising a CDR1 provided by SEQ ID NO: 531, a CDR2 provided by SEQ ID NO: 532, and a CDR3 provided by SEQ ID NO: 533.

In some embodiments, the anti-CLL-1 antibody, or antigen-binding fragment thereof, comprises: a heavy chain variable region comprising a CDR1 provided by SEQ ID NO: 543, a CDR2 provided by SEQ ID NO: 544, and a CDR3 provided by SEQ ID NO: 545; and a light chain variable region comprising a CDR1 provided by SEQ ID NO: 539, a CDR2 provided by SEQ ID NO: 540, and a CDR3 provided by SEQ ID NO: 541.

In some embodiments, the anti-CLL-1 antibody, or antigen-binding fragment thereof, comprises: a heavy chain variable region comprising a CDR1 provided by SEQ ID NO: 551, a CDR2 provided by SEQ ID NO: 552, and a CDR3 provided by SEQ ID NO: 553; and a light chain variable region comprising a CDR1 provided by SEQ ID NO: 547, a CDR2 provided by SEQ ID NO: 548, and a CDR3 provided by SEQ ID NO: 549.

In some embodiments, the anti-CLL-1 antibody, or antigen-binding fragment thereof, comprises: a heavy chain variable region comprising a CDR1 provided by SEQ ID NO: 559, a CDR2 provided by SEQ ID NO: 560, and a CDR3 provided by SEQ ID NO: 561; and a light chain variable region comprising a CDR1 provided by SEQ ID NO: 555, a CDR2 provided by SEQ ID NO: 556, and a CDR3 provided by SEQ ID NO: 557.

In some embodiments, the anti-CLL-1 antibody, or antigen-binding fragment thereof, comprises: a heavy chain variable region comprising a CDR1 provided by SEQ ID NO: 567, a CDR2 provided by SEQ ID NO: 568, and a CDR3 provided by SEQ ID NO: 569; and a light chain variable region comprising a CDR1 provided by SEQ ID NO: 563, a CDR2 provided by SEQ ID NO: 564, and a CDR3 provided by SEQ ID NO: 565.

In some embodiments, the anti-CLL-1 antibody, or antigen-binding fragment thereof, comprises: a heavy chain variable region comprising a CDR1 provided by SEQ ID NO: 575, a CDR2 provided by SEQ ID NO: 576, and a CDR3 provided by SEQ ID NO: 577; and a light chain variable region comprising a CDR1 provided by SEQ ID NO: 571, a CDR2 provided by SEQ ID NO: 572, and a CDR3 provided by SEQ ID NO: 573.

In some embodiments, the anti-CLL-1 antibody, or antigen-binding fragment thereof, comprises: a heavy chain variable region comprising a CDR1 provided by SEQ ID NO: 583, a CDR2 provided by SEQ ID NO: 584, and a CDR3 provided by SEQ ID NO: 585; and a light chain variable region comprising a CDR1 provided by SEQ ID NO: 579, a CDR2 provided by SEQ ID NO: 580, and a CDR3 provided by SEQ ID NO: 581.

In some embodiments, the anti-CLL-1 antibody, or antigen-binding fragment thereof comprises: a heavy chain variable region comprising a CDR1 provided by SEQ ID NO: 591, a CDR2 provided by SEQ ID NO: 592, and a CDR3 provided by SEQ ID NO: 593; and a light chain variable region comprising a CDR1 provided by SEQ ID NO: 591, a CDR2 provided by SEQ ID NO: 592, and a CDR3 provided by SEQ ID NO: 593.

In some embodiments, the anti-CLL-1 antibody, or antigen-binding fragment thereof comprises: a heavy chain variable region comprising a CDR1 provided by SEQ ID NO: 599, a CDR2 provided by SEQ ID NO: 600, and a CDR3 provided by SEQ ID NO: 601; and a light chain variable region comprising a CDR1 provided by SEQ ID NO: 595, a CDR2 provided by SEQ ID NO: 596, and a CDR3 provided by SEQ ID NO: 597.

In some embodiments, the anti-CLL-1 antibody, or antigen-binding fragment thereof, comprises: a heavy chain variable region comprising a CDR1 provided by SEQ ID NO: 607, a CDR2 provided by SEQ ID NO: 608, and a CDR3 provided by SEQ ID NO: 609; and a light chain variable region comprising a CDR1 provided by SEQ ID NO: 603, a CDR2 provided by SEQ ID NO: 604, and a CDR3 provided by SEQ ID NO: 605.

In some embodiments, the anti-CLL-1 antibody, or antigen-binding fragment thereof, comprises: a heavy chain variable region comprising a CDR1 provided by SEQ ID NO: 615, a CDR2 provided by SEQ ID NO: 616, and a CDR3 provided by SEQ ID NO: 617; and a light chain variable region comprising a CDR1 provided by SEQ ID NO: 611, a CDR2 provided by SEQ ID NO: 612, and a CDR3 provided by SEQ ID NO: 613.

In some embodiments, the anti-CLL-1 antibody, or antigen-binding fragment thereof, comprises: a heavy chain variable region comprising a CDR1 provided by SEQ ID NO: 623, a CDR2 provided by SEQ ID NO: 624, and a CDR3 provided by SEQ ID NO: 625; and a light chain variable region comprising a CDR1 provided by SEQ ID NO: 619, a CDR2 provided by SEQ ID NO: 620, and a CDR3 provided by SEQ ID NO: 621.

In some embodiments, the anti-CLL-1 antibody, or antigen-binding fragment thereof, comprises: a heavy chain variable region comprising a CDR1 provided by SEQ ID NO: 631, a CDR2 provided by SEQ ID NO: 632, and a CDR3 provided by SEQ ID NO: 633; and a light chain variable region comprising a CDR1 provided by SEQ ID NO: 627, a CDR2 provided by SEQ ID NO: 628, and a CDR3 provided by SEQ ID NO: 629.

In some embodiments, the anti-CLL-1 antibody, or antigen-binding fragment thereof, comprises: a heavy chain variable region comprising a CDR1 provided by SEQ ID NO: 639, a CDR2 provided by SEQ ID NO: 640, and a CDR3 provided by SEQ ID NO: 641; and a light chain variable region comprising a CDR1 provided by SEQ ID NO: 635, a CDR2 provided by SEQ ID NO: 636, and a CDR3 provided by SEQ ID NO: 637.

In some embodiments, the anti-CLL-1 antibody, or antigen-binding fragment thereof, comprises: a heavy chain variable region comprising a CDR1 provided by SEQ ID NO: 647, a CDR2 provided by SEQ ID NO: 648, and a CDR3 provided by SEQ ID NO: 649; and a light chain variable region comprising a CDR1 provided by SEQ ID NO: 643, a CDR2 provided by SEQ ID NO: 644, and a CDR3 provided by SEQ ID NO: 645.

In some embodiments, the anti-CLL-1 antibody, or antigen-binding fragment thereof, comprises: a heavy chain variable region comprising a CDR1 provided by SEQ ID NO: 655, a CDR2 provided by SEQ ID NO: 656, and a CDR3 provided by SEQ ID NO: 657; and a light chain variable region comprising a CDR1 provided by SEQ ID NO: 651, a CDR2 provided by SEQ ID NO: 652, and a CDR3 provided by SEQ ID NO: 653.

In some embodiments, the anti-CLL-1 antibody, or antigen-binding fragment thereof, comprises: a heavy chain variable region comprising a CDR1 provided by SEQ ID NO: 663, a CDR2 provided by SEQ ID NO: 664, and a CDR3 provided by SEQ ID NO: 665; and a light chain variable region comprising a CDR1 provided by SEQ ID NO: 659, a CDR2 provided by SEQ ID NO: 659, and a CDR3 provided by SEQ ID NO: 661.

In some embodiments, the anti-CLL-1 antibody, or antigen-binding fragment thereof, comprises: a heavy chain variable region comprising a CDR1 provided by SEQ ID NO: 671, a CDR2 provided by SEQ ID NO: 672, and a CDR3 provided by SEQ ID NO: 673; and a light chain variable region comprising a CDR1 provided by SEQ ID NO: 667, a CDR2 provided by SEQ ID NO: 668, and a CDR3 provided by SEQ ID NO: 669.

In some embodiments, the anti-CLL-1 antibody, or antigen-binding fragment thereof, comprises: a heavy chain variable region comprising a CDR1 provided by SEQ ID NO: 679, a CDR2 provided by SEQ ID NO: 680, and a CDR3 provided by SEQ ID NO: 681; and a light chain variable region comprising a CDR1 provided by SEQ ID NO: 675, a CDR2 provided by SEQ ID NO: 676, and a CDR3 provided by SEQ ID NO: 677.

In some embodiments, the anti-CLL-1 antibody, or antigen-binding fragment thereof, comprises: a heavy chain variable region comprising a CDR1 provided by SEQ ID NO: 687, a CDR2 provided by SEQ ID NO: 688, and a CDR3 provided by SEQ ID NO: 689; and a light chain variable region comprising a CDR1 provided by SEQ ID NO: 683, a CDR2 provided by SEQ ID NO: 684, and a CDR3 provided by SEQ ID NO: 685.

In some embodiments, the anti-CLL-1 antibody, or antigen-binding fragment thereof, comprises: a heavy chain variable region comprising a CDR1 provided by SEQ ID NO: 695, a CDR2 provided by SEQ ID NO: 696, and a CDR3 provided by SEQ ID NO: 697; and a light chain variable region comprising a CDR1 provided by SEQ ID NO: 691, a CDR2 provided by SEQ ID NO: 692, and a CDR3 provided by SEQ ID NO: 693.

In some embodiments, the anti-CLL-1 antibody, or antigen-binding fragment thereof, comprises: a heavy chain variable region comprising a CDR1 provided by SEQ ID NO: 703, a CDR2 provided by SEQ ID NO: 704, and a CDR3 provided by SEQ ID NO: 705; and a light chain variable region comprising a CDR1 provided by SEQ ID NO: 699, a CDR2 provided by SEQ ID NO: 700, and a CDR3 provided by SEQ ID NO: 701.

In some embodiments, the anti-CLL-1 antibody, or antigen-binding fragment thereof, comprises: a heavy chain variable region comprising a CDR1 provided by SEQ ID NO: 711, a CDR2 provided by SEQ ID NO: 712, and a CDR3 provided by SEQ ID NO: 713; and a light chain variable region comprising a CDR1 provided by SEQ ID NO: 707, a CDR2 provided by SEQ ID NO: 708, and a CDR3 provided by SEQ ID NO: 709.

In some embodiments, the anti-CLL-1 antibody, or antigen-binding fragment thereof, comprises a heavy chain variable region comprising a CDR1 provided by SEQ ID NO: 823, a CDR2 provided by SEQ ID NO: 824, and a CDR3 provided by SEQ ID NO: 825; and a light chain variable region comprising a CDR1 provided by SEQ ID NO: 820, a CDR2 provided by SEQ ID NO: 821, and a CDR3 provided by SEQ ID NO: 822.

In some embodiments, the anti-CLL-1 antibody, or antigen-binding fragment thereof, comprises a heavy chain variable region comprising a CDR1 provided by SEQ ID NO: 841, a CDR2 provided by SEQ ID NO: 842, and a CDR3 provided by SEQ ID NO: 843; and a light chain variable region comprising a CDR1 provided by SEQ ID NO: 838, a CDR2 provided by SEQ ID NO: 839, and a CDR3 provided by SEQ ID NO: 840.

In some embodiments, the anti-CLL-1 antibody, or antigen-binding fragment thereof, comprises a heavy chain variable region comprising a CDR1 provided by SEQ ID NO: 852, a CDR2 provided by SEQ ID NO: 853, and a CDR3 provided by SEQ ID NO: 854; and a light chain variable region comprising a CDR1 provided by SEQ ID NO: 850, a CDR2 provided by SEQ ID NO: 839, and a CDR3 provided by SEQ ID NO: 851.

In some embodiments, the anti-CLL-1 antibody, or antigen-binding fragment thereof, comprises a heavy chain variable region comprising a CDR1 provided by SEQ ID NO: 914, a CDR2 provided by SEQ ID NO: 915, and a CDR3 provided by SEQ ID NO: 916; and a light chain variable region comprising a CDR1 provided by SEQ ID NO: 911, a CDR2 provided by SEQ ID NO: 912, and a CDR3 provided by SEQ ID NO: 913.

In some embodiments, the anti-CLL-1 antibody, or antigen-binding fragment thereof, comprises a CDR1 provided by SEQ ID NO: 715, a CDR2 provided by SEQ ID NO: 716, and a CDR3 provided by SEQ ID NO: 717. In some embodiments, the anti-CLL-1 antibody, or antigen-binding fragment thereof, comprises a CDR1 provided by SEQ ID NO: 719, a CDR2 provided by SEQ ID NO: 720, and a CDR3 provided by SEQ ID NO: 721. In some embodiments, the anti-CLL-1 antibody, or antigen-binding fragment thereof, comprises a CDR1 provided by SEQ ID NO: 723, a CDR2 provided by SEQ ID NO: 724, and a CDR3 provided by SEQ ID NO: 725. In some embodiments, the anti-CLL-1 antibody, or antigen-binding fragment thereof, comprises a CDR1 provided by SEQ ID NO: 727, a CDR2 provided by SEQ ID NO: 728, and a CDR3 provided by SEQ ID NO: 729. In some embodiments, the anti-CLL-1 antibody, or antigen-binding fragment thereof, comprises a CDR1 provided by SEQ ID NO: 731, a CDR2 provided by SEQ ID NO: 732, and a CDR3 provided by SEQ ID NO: 733. In some embodiments, the anti-CLL-1 antibody, or antigen-binding fragment thereof, comprises a CDR1 provided by SEQ ID NO: 739, a CDR2 provided by SEQ ID NO: 740, and a CDR3 provided by SEQ ID NO: 741.

In some embodiments, the anti-CLL-1 antibody, or antigen-binding fragment thereof, comprises a CDR1 provided by SEQ ID NO: 743, a CDR2 provided by SEQ ID NO: 744, and a CDR3 provided by SEQ ID NO: 745. In some embodiments, the anti-CLL-1 antibody, or antigen-binding fragment thereof, comprises a CDR1 provided by SEQ ID NO: 747, a CDR2 provided by SEQ ID NO: 748, and a CDR3 provided by SEQ ID NO: 749. In some embodiments, the anti-CLL-1 antibody, or antigen-binding fragment thereof, comprises a CDR1 provided by SEQ ID NO: 751, a CDR2 provided by SEQ ID NO: 752, and a CDR3 provided by SEQ ID NO: 753. In some embodiments, the anti-CLL-1 antibody, or antigen-binding fragment thereof, comprises a CDR1 provided by SEQ ID NO: 755, a CDR2 provided by SEQ ID NO: 756, and a CDR3 provided by SEQ ID NO: 757. In some embodiments, the anti-CLL-1 antibody, or antigen-binding fragment thereof, comprises a CDR1 provided by SEQ ID NO: 759, a CDR2 provided by SEQ ID NO: 760, and a CDR3 provided by SEQ ID NO: 761. In some embodiments, the anti-CLL-1 antibody, or antigen-binding fragment thereof, comprises a CDR1 provided by SEQ ID NO: 763, a CDR2 provided by SEQ ID NO: 764, and a CDR3 provided by SEQ ID NO: 765. In some embodiments, the anti-CLL-1 antibody, or antigen-binding fragment thereof, comprises a CDR1 provided by SEQ ID NO: 767, a CDR2 provided by SEQ ID NO: 768, and a CDR3 provided by SEQ ID NO: 769. In some embodiments, the anti-CLL-1 antibody, or antigen-binding fragment thereof, comprises a CDR1 provided by SEQ ID NO: 770, a CDR2 provided by SEQ ID NO: 771, and a CDR3 provided by SEQ ID NO: 772. In some embodiments, the anti-CLL-1 antibody, or antigen-binding fragment thereof, comprises a CDR1 provided by SEQ ID NO: 775, a CDR2 provided by SEQ ID NO: 776, and a CDR3 provided by SEQ ID NO: 777. In some embodiments, the anti-CLL-1 antibody, or antigen-binding fragment thereof, comprises a CDR1 provided by SEQ ID NO: 780, a CDR2 provided by SEQ ID NO: 781, and a CDR3 provided by SEQ ID NO: 782. In some embodiments, the anti-CLL-1 antibody, or antigen-binding fragment thereof, comprises a CDR1 provided by SEQ ID NO: 785, a CDR2 provided by SEQ ID NO: 786, and a CDR3 provided by SEQ ID NO: 777.

In some embodiments, the anti-CLL-1 antibody, or antigen-binding fragment thereof, comprises a CDR1 provided by SEQ ID NO: 789, a CDR2 provided by SEQ ID NO: 790, and a CDR3 provided by SEQ ID NO: 791.

In some embodiments, the anti-CLL-1 antibody, or antigen-binding fragment thereof, comprises a CDR1 provided by SEQ ID NO: 794, a CDR2 provided by SEQ ID NO: 795, and a CDR3 provided by SEQ ID NO: 796. In some embodiments, the anti-CLL-1 antibody, or antigen-binding fragment thereof, comprises a CDR1 provided by SEQ ID NO: 785, a CDR2 provided by SEQ ID NO: 799, and a CDR3 provided by SEQ ID NO: 777. In some embodiments, the anti-CLL-1 antibody, or antigen-binding fragment thereof, comprises a CDR1 provided by SEQ ID NO: 785, a CDR2 provided by SEQ ID NO: 802, and a CDR3 provided by SEQ ID NO: 777. In some embodiments, the anti-CLL-1 antibody, or antigen-binding fragment thereof, comprises a CDR1 provided by SEQ ID NO: 789, a CDR2 provided by SEQ ID NO: 805, and a CDR3 provided by SEQ ID NO: 806. In some embodiments, the anti-CLL-1 antibody, or antigen-binding fragment thereof, comprises a CDR1 provided by SEQ ID NO: 901, a CDR2 provided by SEQ ID NO: 902, and a CDR3 provided by SEQ ID NO: 903. In some embodiments, the anti-CLL-1 antibody, or antigen-binding fragment thereof, comprises a CDR1 provided by SEQ ID NO: 906, a CDR2 provided by SEQ ID NO: 907, and a CDR3 provided by SEQ ID NO: 908.

In another aspect, the disclosure is directed to an anti-CLL-1 antibody, or antigen-binding fragment thereof, comprising a VHH comprising a CDR1 provided by SEQ ID NO: 5, 19, 33, 47, 61, 75, 89, 103, 117, 131, 145, 159, 170, 175, 180, 185, 190, 195, 200, 205, 210, 215, 220, 225, 230, 235, 240, 245, 250, 255, 260, 265, 271, 276, 281, 286, 291, 299, 313, 327, 341, 355, 423, 431, 439, 447, 455, 463, 471, 479, 487, 495, 503, 511, 519, 527, 535, 543, 551, 559, 567, 575, 583, 591, 599, 607, 615, 623, 631, 639, 647, 655, 663, 671, 679, 687, 695, 703, 711, 715, 719, 723, 727, 731, 735, 739, 743, 747, 751, 755, 759, 763, 767, 770, 775, 780, 785, 789, 794, 823, 841, 852, 896, 901, 906, or 914; a CDR2 provided by SEQ ID NO: 6, 20, 34, 48, 62, 76, 90, 104, 118, 132, 146, 160, 171, 176, 181, 186, 191, 196, 201, 206, 211, 216, 221, 226, 231, 236, 241, 246, 251, 256, 261, 266, 272, 277, 282, 287, 292, 300, 314, 328, 342, 356, 424, 432, 440, 448, 456, 464, 472, 480, 488, 496, 504, 512, 520, 528, 536, 544, 552, 560, 568, 576, 584, 592, 600, 608, 616, 624, 632, 640, 648, 656, 664, 672, 680, 688, 696, 704, 712, 716, 720, 724, 728, 732, 736, 740, 744, 748, 752, 756, 760, 764, 768, 771, 776, 781, 786, 790, 795, 799, 802, 805, 824, 842, 853, 897, 902, 907, or 915; and a CDR3 provided by SEQ ID NO: 7, 21, 35, 49, 63, 77, 91, 105, 119, 133, 147, 161, 172, 177, 182, 187, 192, 197, 202, 207, 212, 217, 222, 227, 232, 237, 242, 247, 252, 257, 262, 267, 273, 278, 283, 288, 293, 301, 315, 329, 343, 357, 425, 433, 441, 449, 457, 465, 473, 481, 489, 497, 505, 513, 521, 529, 537, 545, 553, 561, 569, 577, 585, 593, 601, 609, 617, 625, 633, 641, 649, 657, 665, 673, 681, 689, 697, 705, 713, 717, 721, 725, 729, 733, 737, 741, 745, 749, 753, 757, 761, 765, 769, 772, 777, 782, 791, 796, 806, 825, 843, 854, 898, 903, 908, or 916.

In some embodiments, the VHH comprises a CDR1 provided by SEQ ID NO: 170, a CDR2 provided by SEQ ID NO: 171, and a CDR3 provided by SEQ ID NO: 172. In some embodiments, the VHH comprises a CDR1 provided by SEQ ID NO: 175, a CDR2 provided by SEQ ID NO: 176, and a CDR3 provided by SEQ ID NO: 177. In some embodiments, the VHH comprises a CDR1 provided by SEQ ID NO: 180, a CDR2 provided by SEQ ID NO: 181, and a CDR3 provided by SEQ ID NO: 182. In some embodiments, the VHH comprises a CDR1 provided by SEQ ID NO: 185, a CDR2 provided by SEQ ID NO: 186, and a CDR3 provided by SEQ ID NO: 187. In some embodiments, the VHH comprises a CDR1 provided by SEQ ID NO: 190, a CDR2 provided by SEQ ID NO: 191, and a CDR3 provided by SEQ ID NO: 192. In some embodiments, the VHH comprises a CDR1 provided by SEQ ID NO: 195, a CDR2 provided by SEQ ID NO: 196, and a CDR3 provided by SEQ ID NO: 197. In some embodiments, the VHH comprises a CDR1 provided by SEQ ID NO: 200, a CDR2 provided by SEQ ID NO: 201, and a CDR3 provided by SEQ ID NO: 202. In some embodiments, the VHH comprises a CDR1 provided by SEQ ID NO: 205, a CDR2 provided by SEQ ID NO: 206, and a CDR3 provided by SEQ ID NO: 207. In some embodiments, the VHH comprises a CDR1 provided by SEQ ID NO: 210, a CDR2 provided by SEQ ID NO: 211, and a CDR3 provided by SEQ ID NO: 212. In some embodiments, the VHH comprises a CDR1 provided by SEQ ID NO: 215, a CDR2 provided by SEQ ID NO: 216, and a CDR3 provided by SEQ ID NO: 217. In some embodiments, the VHH comprises a CDR1 provided by SEQ ID NO: 220, a CDR2 provided by SEQ ID NO: 221, and a CDR3 provided by SEQ ID NO: 222. In some embodiments, the VHH comprises a CDR1 provided by SEQ ID NO: 225, a CDR2 provided by SEQ ID NO: 226, and a CDR3 provided by SEQ ID NO: 227. In some embodiments, the VHH comprises a CDR1 provided by SEQ ID NO: 230, a CDR2 provided by SEQ ID NO: 231, and a CDR3 provided by SEQ ID NO: 232. In some embodiments, the VHH comprises a CDR1 provided by SEQ ID NO: 235, a CDR2 provided by SEQ ID NO: 236, and a CDR3 provided by SEQ ID NO: 237. In some embodiments, the VHH comprises a CDR1 provided by SEQ ID NO: 240, a CDR2 provided by SEQ ID NO: 241, and a CDR3 provided by SEQ ID NO: 242. In some embodiments, the VHH comprises a CDR1 provided by SEQ ID NO: 245, a CDR2 provided by SEQ ID NO: 246, and a CDR3 provided by SEQ ID NO: 247. In some embodiments, the VHH comprises a CDR1 provided by SEQ ID NO: 250, a CDR2 provided by SEQ ID NO: 251, and a CDR3 provided by SEQ ID NO: 252. In some embodiments, the VHH comprises a CDR1 provided by SEQ ID NO: 255, a CDR2 provided by SEQ ID NO: 256, and a CDR3 provided by SEQ ID NO: 257. In some embodiments, the VHH comprises a CDR1 provided by SEQ ID NO: 260, a CDR2 provided by SEQ ID NO: 261, and a CDR3 provided by SEQ ID NO: 262. In some embodiments, the VHH comprises a CDR1 provided by SEQ ID NO: 265, a CDR2 provided by SEQ ID NO: 266, and a CDR3 provided by SEQ ID NO: 267. In some embodiments, the VHH comprises a CDR1 provided by SEQ ID NO: 271, a CDR2 provided by SEQ ID NO: 272, and a CDR3 provided by SEQ ID NO: 273. In some embodiments, the VHH comprises a CDR1 provided by SEQ ID NO: 276, a CDR2 provided by SEQ ID NO: 277, and a CDR3 provided by SEQ ID NO: 278. In some embodiments, the VHH comprises a CDR1 provided by SEQ ID NO: 281, a CDR2 provided by SEQ ID NO: 282, and a CDR3 provided by SEQ ID NO: 283. In some embodiments, the VHH comprises a CDR1 provided by SEQ ID NO: 286, a CDR2 provided by SEQ ID NO: 287, and a CDR3 provided by SEQ ID NO: 288. In some embodiments, the VHH comprises a CDR1 provided by SEQ ID NO: 291, a CDR2 provided by SEQ ID NO: 292, and a CDR3 provided by SEQ ID NO: 293. In some embodiments, the VHH comprises a CDR1 provided by SEQ ID NO: 715, a CDR2 provided by SEQ ID NO: 716, and a CDR3 provided by SEQ ID NO: 717. In some embodiments, the VHH comprises a CDR1 provided by SEQ ID NO: 719, a CDR2 provided by SEQ ID NO: 720, and a CDR3 provided by SEQ ID NO: 721. In some embodiments, the VHH comprises a CDR1 provided by SEQ ID NO: 723, a CDR2 provided by SEQ ID NO: 724, and a CDR3 provided by SEQ ID NO: 725. In some embodiments, the VHH comprises a CDR1 provided by SEQ ID NO: 727, a CDR2 provided by SEQ ID NO: 728, and a CDR3 provided by SEQ ID NO: 729. In some embodiments, the VHH comprises a CDR1 provided by SEQ ID NO: 731, a CDR2 provided by SEQ ID NO: 732, and a CDR3 provided by SEQ ID NO: 733. In some embodiments, the VHH comprises a CDR1 provided by SEQ ID NO: 735, a CDR2 provided by SEQ ID NO: 736, and a CDR3 provided by SEQ ID NO: 737. In some embodiments, the VHH comprises a CDR1 provided by SEQ ID NO: 739, a CDR2 provided by SEQ ID NO: 740, and a CDR3 provided by SEQ ID NO: 741. In some embodiments, the VHH comprises a CDR1 provided by SEQ ID NO: 743, a CDR2 provided by SEQ ID NO: 744, and a CDR3 provided by SEQ ID NO: 745. In some embodiments, the VHH comprises a CDR1 provided by SEQ ID NO: 747, a CDR2 provided by SEQ ID NO: 748, and a CDR3 provided by SEQ ID NO: 749. In some embodiments, the VHH comprises a CDR1 provided by SEQ ID NO: 751, a CDR2 provided by SEQ ID NO: 752, and a CDR3 provided by SEQ ID NO: 753. In some embodiments, the VHH comprises a CDR1 provided by SEQ ID NO: 755, a CDR2 provided by SEQ ID NO: 756, and a CDR3 provided by SEQ ID NO: 757. In some embodiments, the VHH comprises a CDR1 provided by SEQ ID NO: 759, a CDR2 provided by SEQ ID NO: 760, and a CDR3 provided by SEQ ID NO: 761. In some embodiments, the VHH comprises a CDR1 provided by SEQ ID NO: 763, a CDR2 provided by SEQ ID NO: 764, and a CDR3 provided by SEQ ID NO: 765. In some embodiments, the VHH comprises a CDR1 provided by SEQ ID NO: 767, a CDR2 provided by SEQ ID NO: 768, and a CDR3 provided by SEQ ID NO: 769.

In some embodiments, the VHH comprises a CDR1 provided by SEQ ID NO: 770, a CDR2 provided by SEQ ID NO: 771, and a CDR3 provided by SEQ ID NO: 772. In some embodiments, the VHH comprises a CDR1 provided by SEQ ID NO: 775, a CDR2 provided by SEQ ID NO: 776, and a CDR3 provided by SEQ ID NO: 777. In some embodiments, the VHH comprises a CDR1 provided by SEQ ID NO: 780, a CDR2 provided by SEQ ID NO: 781, and a CDR3 provided by SEQ ID NO: 782. In some embodiments, the VHH comprises a CDR1 provided by SEQ ID NO: 785, a CDR2 provided by SEQ ID NO: 786, and a CDR3 provided by SEQ ID NO: 777. In some embodiments, the VHH comprises a a CDR1 provided by SEQ ID NO: 789, a CDR2 provided by SEQ ID NO: 790, and a CDR3 provided by SEQ ID NO: 791. In some embodiments, the VHH comprises a CDR1 provided by SEQ ID NO: 794, a CDR2 provided by SEQ ID NO: 795, and a CDR3 provided by SEQ ID NO: 796. In some embodiments, the VHH comprises a CDR1 provided by SEQ ID NO: 785, a CDR2 provided by SEQ ID NO: 799, and a CDR3 provided by SEQ ID NO: 777. In some embodiments, the VHH comprises a CDR1 provided by SEQ ID NO: 785, a CDR2 provided by SEQ ID NO: 802, and a CDR3 provided by SEQ ID NO: 777. In some embodiments, the VHH comprises a CDR1 provided by SEQ ID NO: 789, a CDR2 provided by SEQ ID NO: 805, and a CDR3 provided by SEQ ID NO: 806. In some embodiments, the VHH comprises a CDR1 provided by SEQ ID NO: 823, a CDR2 provided by SEQ ID NO: 824, and a CDR3 provided by SEQ ID NO: 825. In some embodiments, the VHH comprises a CDR1 provided by SEQ ID NO: 823, a CDR2 provided by SEQ ID NO: 824, and a CDR3 provided by SEQ ID NO: 825. In some embodiments, the VHH comprises a CDR1 provided by SEQ ID NO: 841, a CDR2 provided by SEQ ID NO: 842, and a CDR3 provided by SEQ ID NO: 843. In some embodiments, the VHH comprises a CDR1 provided by SEQ ID NO: 852, a CDR2 provided by SEQ ID NO: 853, and a CDR3 provided by SEQ ID NO: 854. In some embodiments, the VHH comprises a CDR1 provided by SEQ ID NO: 896, a CDR2 provided by SEQ ID NO: 897, and a CDR3 provided by SEQ ID NO: 898. In some embodiments, the VHH comprises a CDR1 provided by SEQ ID NO: 901, a CDR2 provided by SEQ ID NO: 902, and a CDR3 provided by SEQ ID NO: 903. In some embodiments, the VHH comprises a CDR1 provided by SEQ ID NO: 906, a CDR2 provided by SEQ ID NO: 907, and a CDR3 provided by SEQ ID NO: 908. In some embodiments, the VHH comprises a CDR1 provided by SEQ ID NO: 914, a CDR2 provided by SEQ ID NO: 915, and a CDR3 provided by SEQ ID NO: 916.

In another aspect, the disclosure is directed to an anti-CLL-1 antibody, or antigen-binding fragment thereof, comprising a VHH comprising an amino acid sequence of SEQ ID NOs: 174, 179, 184, 189, 194, 199, 204, 209, 214, 219, 224, 229, 234, 239, 244, 249, 254, 259, 264, 269, 275, 280, 285, 290, 295, 714, 718, 722, 726, 730, 734, 738, 742, 746, 750, 754, 758, 762, or 766.

In another aspect, the disclosure is directed to an anti-CLL-1 antibody, or antigen-binding fragment thereof, comprising a VHH comprising a CDR sequence of SEQ ID NOs: 5-7, 19-21, 33-35, 47-49, 61-63, 75-77, 89-91, 103-105, 117-119, 131-133, 145-147, 159-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 299-301, 313-315, 327-329, 341-343, 355-357, 423-425, 431-433, 439-441, 447-449, 455-457, 463-465, 471-473, 479-481, 487-489, 495-497, 503-505, 511-513, 519-521, 527-529, 535-537, 543-545, 551-553, 559-561, 567-569, 575-577, 583-585, 591-593, 599-601, 607-609, 615-617, 623-625, 631-633, 639-641, 647-649, 655-657, 663-665, 671-673, 679-681, 687-689, 695-697, 703-705, 711-713, 715-717, 719-821, 723-725, 727-729, 731-733, 735-737, 739-741, 743-745, 747-749, 751-753, 755-757, 759-761, 763-765, 767-772, 775-777, 780-782, 785-786, 789-791, 794-796, 799, 802, 805-806, 823-825, 841-843, 852-854, 896-898, 901-903, 906-908, or 914-916.

In another aspect, the disclosure is directed to an anti-CLL-1 antibody, or antigen-binding fragment thereof, comprising a VHH comprising a CDR1, a CDR2, and a CDR3 of SEQ ID NOs: 5-7, 19-21, 33-35, 47-49, 61-63, 75-77, 89-91, 103-105, 117-119, 131-133, 145-147, 159-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 299-301, 313-315, 327-329, 341-343, 355-357, 423-425, 431-433, 439-441, 447-449, 455-457, 463-465, 471-473, 479-481, 487-489, 495-497, 503-505, 511-513, 519-521, 527-529, 535-537, 543-545, 551-553, 559-561, 567-569, 575-577, 583-585, 591-593, 599-601, 607-609, 615-617, 623-625, 631-633, 639-641, 647-649, 655-657, 663-665, 671-673, 679-681, 687-689, 695-697, 703-705, 711-713, 715-717, 719-821, 723-725, 727-729, 731-733, 735-737, 739-741, 743-745, 747-749, 751-753, 755-757, 759-761, 763-765, 767-772, 775-777, 780-782, 785-786, 789-791, 794-796, 799, 802, 805-806, 823-825, 841-843, 852-854, 896-898, 901-903, 906-908, or 914-916.

In another aspect, the disclosure is directed to an anti-CLL-1 antibody, or antigen-binding fragment thereof, comprising a VHH comprising at least one CDR (e.g., CDR1, CDR2, and/or CDR3) of SEQ ID NOs: 5-7, 19-21, 33-35, 47-49, 61-63, 75-77, 89-91, 103-105, 117-119, 131-133, 145-147, 159-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 299-301, 313-315, 327-329, 341-343, 355-357, 423-425, 431-433, 439-441, 447-449, 455-457, 463-465, 471-473, 479-481, 487-489, 495-497, 503-505, 511-513, 519-521, 527-529, 535-537, 543-545, 551-553, 559-561, 567-569, 575-577, 583-585, 591-593, 599-601, 607-609, 615-617, 623-625, 631-633, 639-641, 647-649, 655-657, 663-665, 671-673, 679-681, 687-689, 695-697, 703-705, 711-713, 715-717, 719-821, 723-725, 727-729, 731-733, 735-737, 739-741, 743-745, 747-749, 751-753, 755-757, 759-761, 763-765, 767-772, 775-777, 780-782, 785-786, 789-791, 794-796, 799, 802, 805-806, 823-825, 841-843, 852-854, 896-898, 901-903, 906-908, or 914-916.

In another aspect, the disclosure is directed to an anti-CLL-1 antibody, or antigen-binding fragment thereof, comprising a VHH comprising at least one CDR that is at least 85%, 90%, 91%, 92%, 93%, 94%, 95%, 96%, 97%, 98%, 99%, or 100% identical to a CDR (e.g., CDR1, CDR2, and/or CDR3) of SEQ ID NOs: 5-7, 19-21, 33-35, 47-49, 61-63, 75-77, 89-91, 103-105, 117-119, 131-133, 145-147, 159-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 299-301, 313-315, 327-329, 341-343, 355-357, 423-425, 431-433, 439-441, 447-449, 455-457, 463-465, 471-473, 479-481, 487-489, 495-497, 503-505, 511-513, 519-521, 527-529, 535-537, 543-545, 551-553, 559-561, 567-569, 575-577, 583-585, 591-593, 599-601, 607-609, 615-617, 623-625, 631-633, 639-641, 647-649, 655-657, 663-665, 671-673, 679-681, 687-689, 695-697, 703-705, 711-713, 715-717, 719-821, 723-725, 727-729, 731-733, 735-737, 739-741, 743-745, 747-749, 751-753, 755-757, 759-761, 763-765, 767-772, 775-777, 780-782, 785-786, 789-791, 794-796, 799, 802, 805-806, 823-825, 841-843, 852-854, 896-898, 901-903, 906-908, or 914-916.

In some embodiments, the antibody, or antigen-binding fragment thereof, is a monoclonal antibody, or antigen-binding fragment thereof. In some embodiments, the antibody, or antigen-binding fragment thereof, is a humanized antibody, or antigen-binding fragment thereof. In another aspect, the disclosure is directed to an anti-CLL-1 antibody, or antigen-binding fragment thereof, that competes for binding to CLL-1 with an antibody, or antigen-binding fragment thereof, described herein. In some embodiments, the antibody, or antigen-binding fragment thereof, comprises a CH2 constant domain and a CH3 constant domain.

In some embodiments, the antibody, or antigen-binding fragment thereof, comprises an amino acid sequence of SEQ ID NO: 15, 29, 43, 57, 71, 85, 99, 113, 127, 141, 155, 169, 174, 179, 184, 189, 194, 199, 204, 209, 214, 219, 224, 229, 234, 239, 244, 249, 254, 259, 264, 269, 275, 280, 285, 290, 295, 309, 323, 337, 351, 365, 418, 422, 426, 430, 434, 438, 442, 446, 450, 454, 458, 462, 466, 470, 474, 478, 482, 486, 490, 494, 498, 502, 506, 510, 514, 518, 522, 526, 530, 534, 538, 542, 546, 550, 554, 558, 562, 566, 570, 574, 578, 582, 586, 590, 584, 598, 602, 606, 610, 614, 618, 622, 626, 630, 634, 638, 642, 646, 650, 654, 658, 662, 666, 670, 674, 678, 682, 686, 690, 694, 698, 702, 706, 710, 714, 718, 722, 726, 730, 734, 738, 742, 746, 750, 754, 758, 762, 766, 774, 779, 784, 788, 793, 798, 801, 804, 808, 817, 829, 831, 835, 837, 847, 849, 858, 860, 893, 900, 905, 910, or 920.

In some embodiments, the antibody, or antigen-binding fragment thereof, is a heavy chain antibody. In some embodiments, the antibody, or antigen-binding fragment thereof, is a camelid antibody.

In some embodiments, the antibody is of the IgG1-, IgG2-, IgG3- or IgG4-type.

In another aspect, the disclosure is directed to a chimeric antigen receptor comprising any of the antibodies, or antigen-binding fragments thereof, described herein.

In another aspect, the disclosure is directed to a cell expressing any of the chimeric antigen receptors described herein. In some embodiments, the cell is an immune effector cell. In some embodiments, the cell is a lymphocyte. In some embodiments, the cell is a T-cell. In some embodiments, the cell is a Natural Killer (NK) cell.

In another aspect, the disclosure is directed to a pharmaceutical composition comprising any of the antibodies described herein, any of the chimeric antigen receptors described herein, or any of the cells described herein, and a pharmaceutically acceptable excipient.

In another aspect, the disclosure is directed to a nucleic acid, comprising a nucleic acid sequence encoding any of the antibodies, or antigen-binding fragment thereof, described herein, or any of the chimeric antigen receptors described herein. In another aspect, the disclosure is directed to a vector comprising any of the nucleic acids described herein.

In another aspect, the disclosure is directed to a cell comprising any of the nucleic acids or vectors described herein. In some embodiments, the nucleic acid comprises a sequence that is 95-99% identical to the sequence of any one of SEQ ID NOs: 8, 10, 12, 14, 22, 24, 26, 28, 36, 38, 40, 42, 50, 52, 54, 56, 64, 66, 68, 70, 78, 80, 82, 84, 92, 94, 96, 98, 106, 108, 110, 112, 120, 122, 124, 126, 134, 136, 138, 140, 148, 150, 152, 154, 162, 164, 166, 168, 173, 178, 183, 188, 193, 198, 203, 208, 213, 218, 223, 228, 233, 238, 243, 248, 253, 258, 263, 268, 274, 279, 284, 289, 294, 302, 304, 306, 308, 316, 318, 320, 322, 330, 332, 334, 336, 344, 346, 348, 350, 358, 360, 362, 364, 773, 778, 783, 787, 792, 797, 800, 803, 807, 826, 828, 830, 832, 834, 836, 844, 846, 848, 855, 857, 859, 904, 909, 917, 919, or 921. In some embodiments, the nucleic acid comprises the sequence of any one of SEQ ID NOs: 8, 10, 12, 14, 22, 24, 26, 28, 36, 38, 40, 42, 50, 52, 54, 56, 64, 66, 68, 70, 78, 80, 82, 84, 92, 94, 96, 98, 106, 108, 110, 112, 120, 122, 124, 126, 134, 136, 138, 140, 148, 150, 152, 154, 162, 164, 166, 168, 173, 178, 183, 188, 193, 198, 203, 208, 213, 218, 223, 228, 233, 238, 243, 248, 253, 258, 263, 268, 274, 279, 284, 289, 294, 302, 304, 306, 308, 316, 318, 320, 322, 330, 332, 334, 336, 344, 346, 348, 350, 358, 360, 362, 364, 773, 778, 783, 787, 792, 797, 800, 803, 807, 826, 828, 830, 832, 834, 836, 844, 846, 848, 855, 857, 859, 904, 909, 917, 919, or 921.

In some embodiments, vector further comprises a promoter sequence that is an SFFV promoter, an EF1α promoter, a tEF1a promoter, a hPGK promoter, a SFFV promoter, or a MND promoter.

In some embodiments, the vector is a DNA vector, an RNA vector, a plasmid, a lentivirus vector, an adenoviral vector or a retrovirus vector.

In some embodiments, the cell is an immune cell. In some embodiments, the immune cell is a T cell, a Natural Killer (NK) cell, a cytotoxic T lymphocyte (CTL), and a regulatory T cell.

In another aspect, the disclosure is directed to a method of producing any of the antibodies, or antigen-binding fragment thereof, comprising culturing a cell described herein under conditions suitable for expression of the antibody or antigen-binding fragment thereof.

In another aspect, the disclosure is directed to a method of treating a CLL-1-associated disease or disorder, the method comprising administering to a subject in need thereof an effective amount of any of the antibodies, or antigen-binding fragment thereof, described herein, any of the antibody drug conjugates described herein, or any of the cells described herein. In some embodiments, the CLL-1-associated disease or disorder is myelodysplastic syndrome (MDS) or acute myeloid leukemia (AML). In some embodiments, the method further comprises administering to the subject an effective amount of a chemotherapeutic agent or an oncolytic therapeutic agent.

In another aspect, the disclosure is directed to CARs comprising a CLL-1 binding domain, a transmembrane domain, and an intracellular signaling domain, wherein the CLL-1 binding domain comprises a heavy chain variable region and/or a light chain variable region; wherein the transmembrane domain comprises a transmembrane domain of a protein selected from CD8a or CD28; and wherein the intracellular signaling domain comprises a signaling domain of CD3ζ. In some embodiments, the heavy chain variable region and the light chain variable region are joined by a linker. In some embodiments, the CLL-1 binding domain comprises at least one of a single-chain variable fragment (scFv), a VH fragment, a VHH fragment, or a VL fragment. In some embodiments, the CLL-1 binding domain is connected to the transmembrane domain by a hinge region. In some embodiments, the hinge region comprises a hinge region of a protein selected from CD8α, IgG4, or CD28.

In some embodiments, the CAR further comprises one or more co-stimulatory domains. In some embodiments, the one more co-stimulatory domains comprises a signaling domain of 4-1BB and/or CD28.

In some embodiments, the CLL-1 binding domain comprises any of the anti-CLL-1 antibody, or antigen-binding fragment thereof, described herein.

In another aspect, the disclosure is directed to a method of treating a subject having or at risk of a neoplastic disease or malignancy of the blood that is associated with CLL-1 expression, the method comprising administering to the subject a therapeutically effective amount of any of the antibodies, or antigen-binding fragment thereof, described herein, any of the antibody drug conjugates described herein, or any of the cells described herein. In some embodiments, the neoplastic disease or malignancy of the blood that is associated with CLL-1 expression is myelodysplastic syndrome (MDS) or acute myeloid leukemia (AML). In some embodiments, the method further comprises administering to the subject an effective amount of a chemotherapeutic agent or an oncolytic therapeutic agent.

In another aspect, the disclosure is directed to a method of treating a subject having or at risk of a neoplastic disease or malignancy of the blood that is associated with CLL-1 expression, the method comprising administering to the subject a therapeutically effective amount of the any of the antibodies, antigen-binding fragment thereof, antibody drug conjugates, chimeric antigens receptors, or cells described herein. In some embodiments, the neoplastic disease or malignancy of the blood that is associated with CLL-1 expression is myelodysplastic syndrome (MDS) or acute myeloid leukemia (AML). In some embodiments, the method further comprises administering to the subject an effective amount of a chemotherapeutic agent or an oncolytic therapeutic agent.

In some embodiments, the method further comprises administering a population of hematopoietic cells, wherein the hematopoietic cells are genetically-engineered such that the gene encoding CLL-1 that is targeted by the antigen-binding domain is engineered to reduce or eliminate the expression of CLL-1. In some embodiments, the immune cells, the hematopoietic cells, or both, are allogeneic or autologous. In some embodiments, the hematopoietic cells are hematopoietic stem cells. In some embodiments, the hematopoietic stem cells are from bone marrow cells or peripheral blood mononuclear cells (PBMCs). In some embodiments, the hematopoietic stem cells are CD34+/CLL-1−. In some embodiments, the hematopoietic cells are prepared by editing the endogenous gene coding for CLL-1 to reduce or eliminate the expression of CLL-1. In some embodiments, the endogenous gene is edited by CRISPR-Cas9.

In some embodiments, the subject has or has been diagnosed with a hematopoietic malignancy or pre-malignancy characterized by the expression of CLL-1 on malignant cells or pre-malignant cells. In some embodiments, the subject has Hodgkin's lymphoma, non-Hodgkin's lymphoma, leukemia, or multiple myeloma. In some embodiments, the leukemia is acute myeloid leukemia, myelodysplastic syndrome, chronic myelogenous leukemia, acute lymphoblastic leukemia, or chronic lymphoblastic leukemia.

### BRIEF DESCRIPTION OF THE FIGURES

FIGS. 1A-1E show flow cytometry analysis plots of target cell-binding ability of CLL-1 antibodies. The cell-binding ability of CLL-1 antibodies was assessed by flow cytometric analysis in HL-60 wildtype (WT) cells (FIG. 1A), U937 cells (FIG. 1B), or HL-60 CLL-1 knockout (KO) control cells (FIG. 1C). In each case for FIGS. 1A-1C, the CLL-1 antibodies were compared to a secondary antibody only control (SEC ONLY). The ability of a control anti-hCLL-1 antibody to bind HL-60 WT (right peak indicated with an asterisk) and HL-60 KO cells (left peak) (FIG. 1D) was assessed by flow cytometry. The ability of a control hCLL-1 antibody to bind U937 cells (FIG. 1E) was assessed by flow cytometry, with the staining of cells with primary and secondary labelled antibodies (right peak indicated with an asterisk) vs. unstained control cells (left peak).

FIGS. 2A and 2B show flow cytometry analysis plots of exemplary NFAT-responsive reporter cell lines as described herein. FIG. 2A shows flow cytometry data of Jurkat cells containing the mOrange reporter molecule under control of the constitutively active E1Falpha promoter and mTurquoise reporter molecule (mTurq) under control of an IL-2 reporter system described herein. Cells were either not activated (“-PMA/Ion,” top row) or activated using phorbol myristate acetate (PMA) and ionomycin (“+PMA/Ion,” bottom row). The left column of plots show cells expressing the mOrange reporter molecule; the middle column shows cells expressing the mTurquoise reporter molecule; and the right column shows cells expressing CD69 (CD69+), an indicator of T cell activation. FIG. 2B shows flow cytometry data of Jurkat cells containing the mTurquoise reporter molecule (mTurq) under control of the constitutively active E1Falpha promoter and mOrange reporter molecule under control of an IL-2 reporter system described herein. Cells were either not activated (“−PMA/Ion,” top row) or activated using phorbol myristate acetate (PMA) and ionomycin (“+PMA/Ion,” bottom row). The left column of plots show cells expressing the mTurquoise reporter molecule; the middle column shows cells expressing the mOrange reporter molecule; and the right column shows cells expressing CD69 (CD69+), an indicator of T cell activation.

FIG. 3 shows a plot quantifying flow cytometric analysis of FIGS. 2A and 2B. The y-axis shows the percentage of cells expressing the second reporter molecule (FR2), which was under control of an IL-2 reporter system described herein, based on the cells expressing the first reporter molecule (FR1), which was under control of the constitutively active promoter EF1a. Cells were either not activated (“−PMA/Ion”) or activated using phorbol myristate acetate (PMA) and ionomycin (“+PMA/Ion”). “EF1a_mOrange_IL-2_mTurq” refers to Jurkat cells containing the mOrange reporter molecule under control of the constitutively active E1Falpha promoter (FR1) and mTurquoise reporter molecule (mTurq) under control of an IL-2 reporter system described herein (FR2). “EF1a_mTurq_IL-2_mOrange” refers to Jurkat cells containing the mTurquoise reporter molecule under control of the constitutively active E1Falpha promoter (FR1) and mOrange reporter molecule under control of an IL-2 reporter system described herein (FR2).

FIG. 4 shows a summary of biochemical and cell-based assays used to characterize anti-CLL-1 antibodies and binders.

FIGS. 5A and 5B show results of enzyme-linked immunosorbent assay (ELSA) analyses of anti-CLL-1 antibody clones. FIG. 5A shows dose-dependent binding of the indicated anti-CLL-1 antibody clones to biotinylated recombinant CLL-1. FIG. 5B shows dose-dependent binding of the indicated anti-CLL-1 binders to biotinylated recombinant CLL-1.

FIGS. 6A-6C show quantification of the specific binding of the indicated anti-CLL-1 binders as calculated from flow cytometric analyses. FIG. 6A shows anti-CLL-1 binders contacted with HL-60 WT cells. FIG. 6B shows anti-CLL-1 binders contacted with CLL-1-positive U937 cells.

FIG. 6C shows anti-CLL-1 binders contacted with CLL-1-negative HEK293 control cells. Dotted line indicates the level obtained from control samples stained with a secondary antibody only.

FIGS. 7A-7C show multi-point flow cytometric analyses of dose-dependent binding of the indicated anti-CLL-1 binders to CLL-1-expressing HL-60 WT cells. FIG. 7A shows cell surface binding analyses of anti-CLL-1 binder clones 1-6. FIG. 7B shows cell surface binding analyses of anti-CLL-1 binder clones 7-12 and 20 VH. FIG. 7C shows cell surface binding analyses of anti-CLL-1 binder clones 38-42

FIGS. 8A and 8B show bar graphs of the reciprocal EC50 values calculated by ELISA analyses of binding of the indicated anti-CLL-1 binders to biotinylated recombinant human CLL-1 extracellular domain protein isoforms (CLL1-K244 or CLL1-Q244). For each binder, the left column corresponds to binding to CLL-1 comprising a lysine at amino acid position 244 (CLL1-K244), the right column corresponds to binding to CLL-1 comprising a glutamine at amino acid position 244 (CLL1-Q244). FIG. 8A shows results from anti-CLL-1 binders identified based on panning with biotinylated recombinant CLL-1 Q244. FIG. 8B shows results from anti-CLL-1 binders identified based on sequential panning with biotinylated recombinant human CLL-1 Q244 and biotinylated recombinant human CLL-1 K244.

FIG. 9 shows a heat map representation of data generated from ELISA analyses of the competitive binding activities of the indicated anti-CLL-1 binder pairs to Fc-tagged recombinant CLL-1 protein extracellular domain.

FIGS. 10A-10C show bio-layer interferometry (BLItz) assay analyses of the indicated anti-CLL-1 binder binding kinetics to biotinylated recombinant CLL-1 protein extracellular domain. FIG. 10A shows plots of the BLItz assay binding analyses of each of the indicated CLL-1 antibody binders. Binding curves for 1 μM of each of the CLL-1 antibody binders are shown. FIG. 10B shows plots of the BLItz assay binding analyses of each of the indicated anti-CLL-1 binders. Binding curves for 1 μM of each of the CLL-1 antibody binders are shown. FIG. 10C shows quantification of EC50 (nM) values as well as Ka and Kd values for each anti-CLL-1 binder calculated from the data in FIGS. 10A and 10B.

FIGS. 11A-11L show results from binding kinetic and affinity assays for the indicated CLL-1 antibody binders to CLL-1 protein, using an Octet® platform. Human recombinant CLL-1 protein was used at 10 g/mL and binders were diluted to 100, 33.33, 11.11, and 3.70 nM to the assay. FIG. 12A shows anti-CLL-1 binder clone 1. FIG. 12B shows anti-CLL-1 binder clone 2. FIG. 12C shows anti-CLL-1 binder clone 3. FIG. 12D shows anti-CLL-1 binder clone 4. FIG. 12E shows anti-CLL-1 binder clone 5. FIG. 12F shows anti-CLL-1 binder clone 6. FIG. 12G shows anti-CLL-1 binder 75. FIG. 12H shows anti-CLL-1 binder clone 8. FIG. 12I shows anti-CLL-1 binder clone 10. FIG. 12J shows anti-CLL-1 binder clone 11. FIG. 12K shows anti-CLL-1 binder clone 12. FIG. 12L shows anti-CLL-1 binder clone 20.

FIGS. 12FA and 12B show biochemical characteristics of the indicated anti-CLL-1 binders. FIG. 12A shows binding affinities of the indicated anti-CLL-1 binders. NB=no binding detect; ND=binding assays not done. FIG. 12B shows a summary of binding characteristics of the anti-CLL-1 binders using the indicated assays.

FIGS. 13A-13C show results from flow cytometry analyses of the indicated anti-CLL-1 VH binders to HEK293 cells expressing CLL-1 variants. FIG. 13A shows binding of the indicated anti-CLL-1 VH binders to HEK293 cells expressing CLL-1 comprising a lysine at amino acid position 244 (293-CLL1-K244). FIG. 13B shows binding of the indicated anti-CLL-1 VH binders to HEK293 cells expressing CLL-1 comprising a glutamine at amino acid position 244 (293-CLL1-Q244). FIG. 13C shows binding of the indicated anti-CLL-1 VH binders to CLL-1-null HEK293 control cell (293-null). Dotted line represents signal from cells stained with the secondary antibody only.

FIG. 14 shows a summary of binding affinity of the indicated CLL-1 binders to CLL-1 (K244) or CLL-1 (Q244) protein isoforms.

FIGS. 15A and 15B show CLL-1-specific activation analysis of Jurkat CD4 T-cells expressing the indicated CLL-1-directed chimeric antigen receptors (CARs), when co-cultured with HL-60 WT target cells, assessed with the IL-2 reporter system (IRS, described in FIG. 2). FIG. 15A shows results from flow cytometry analyses of CD69 and FR2 expression following co-culture of the indicated CLL-1 CAR-T cells and HL-60 WT cells. Delta percentage (Delta %) was calculated by subtracting CD69 and FR2 expression percentages in CLL-1 CAR-T cells co-cultured with HL-60 CLL-1 KO cells (background signal) from CD69 and FR2 expression percentages in CLL-1 CAR-T cells co-cultured with HL-60 WT cells (specific signal). N=2. For each CLL-1 CAR T binder, the left points correspond to CD69 expression and right points correspond to FR2 expression. FIG. 15B shows results from IncuCyte live cell imaging analyses of FR2 levels in CLL-1 CAR-T cells co-cultured with HL-60 WT cells. N=1.

FIGS. 16A-16G show analyses of in vitro co-cultures comprising CLL-1-positive AML cell lines and CLL-1-directed CAR-T cells containing the indicated CLL-1 binders. FIG. 16A shows the transduction efficiency of primary T cells for each of the CLL-1 CAR constructs, as determined by anti-human IgG (H+L) reactivity by flow cytometry. FIG. 16B shows binding of the indicated CLL-1 CAR-T cells to target cells expressing either CLL-1 K244 (K) or CLL-1 Q244 (Q) isoforms, as determined by flow cytometry. N=2. FIG. 16C shows the change in viability of CFSE-labeled HL-60 WT cells or CLL1 KO HL-60 target cells following co-culture with indicated CLL-1 directed CAR-T cells. Viability was normalized to donor matched untransduced control cells (UTD). N=2. FIG. 16D shows CLL-1-directed CAR-T cell activation based on CD25 and CD69 expression (CD25+CD69+) assessed by flow cytometry following co-culture of the CAR-T cells with HL-60 WT cells, HL-60 KO cells, or the effector cells alone. N=2. FIG. 16E shows levels of secreted IL-2 produced following co-culture of CLL-1 CAR-T cells with HL-60 WT cells, HL-60 KO cells, or the effector cells alone. FIG. 16F shows levels of secreted IFN-γ produced following co-culture of CLL-1 CAR-T cells with HL-60 WT cells, HL-60 KO cells, or the effector cells alone. FIG. 16G shows levels of secreted TNF-α produced following co-culture of CLL-1 CAR-T cells with HL-60 WT cells, HL-60 KO cells, or the effector cells alone. For each of the indicated CLL-1 CAR-T cells in FIGS. 18E-18G, the columns refer to effectors alone, CLL1 KO HL60, and WT HL60, from left to right.

FIGS. 17A-17H show analyses of in vitro co-cultures comprising CLL-1-positive AML cell lines and CLL-1-specific CAR-T cells. FIG. 17A shows transduction efficiency of primary T cells for each of the CLL-1-specific CAR constructs, as determined by Protein L reactivity. FIG. 17B shows binding of the indicated CLL-1-specific CAR-T cells to target cells comprising either CLL-1 K244 (CLL1 K) or CLL-1 Q244 (CLL1 Q) protein isoforms, as determined by flow cytometry. N=2. FIG. 17C shows the change in cell viability of CFSE-labeled HL-60 WT cells or HL-60 CLL-1 KO target cells following 24 hour co-culture with CLL-1-specific CAR-T cells. Viability was normalized to donor-matched UTD control cells. N=3. FIG. 17D shows the change in cell viability of CFSE-labeled HL-60 WT cells or HL-60 CLL-1 KO target cells following 48 hour co-culture with CLL-1-specific CAR-T cells. Viability was normalized to donor-matched UTD control cells. N=2. FIG. 17E shows CLL-1-specific CAR-T cell activation based on CD25 and CD69 expression (CD25+CD69+) assessed by flow cytometry following co-culture of the CAR T cells with HL-60 WT cells, HL-60 CLL-1 KO cells, or effector cells alone (CAR alone). N=3. FIG. 17F shows levels of secreted IFN-γ cytokine produced following co-culture of CLL-1-specific CAR-T cells with HL-60 WT cells, HL-60 CLL-1 KO cells, or effector cells along. FIG. 17G shows levels of secreted IL-2 cytokine produced following co-culture of CLL-1-specific CAR-T cells with HL-60 WT cells, HL-60 CLL-1 KO cells, or effector cells along. FIG. 17H shows levels of secreted TNF-α cytokine produced following co-culture of CLL-1-specific CAR-T cells with HL-60 WT cells, HL-60 CLL-1 KO cells, or effector cells along. For FIGS. 19F-19H, N=1. For each of the indicated CLL-1-specific CAR-T cell, the columns refer to WT HL60, CLL1 KO HL60, and effectors alone, from left to right.

### DETAILED DESCRIPTION

The present disclosure is based, in part, on the discovery of novel agents that selectively bind to CLL-1. In some embodiments, the agents are antibodies that selectively bind to CLL-1. In some embodiments, the antibodies comprise a heavy chain variable domain. In some embodiments, the antibodies are single-domain antibodies. In some embodiments, the antibodies comprise a heavy chain variable domain and a light chain variable domain. In some embodiments, the antibodies comprise a heavy chain variable domain and one or more constant domains. The present disclosure also describes chimeric antigen receptors that selectively bind to CLL-1. The disclosure also relates to nucleic acids encoding said antibodies or chimeric antigen receptors, methods of producing said antibodies or chimeric antigen receptors, and methods of use in the treatment of treat malignancies using the same (e.g., acute myeloid leukemia (AML), myelodysplastic syndrome (MDS)).

### Antibodies

The term “antibody” is used herein in the broadest sense and encompasses various antibody structures, including but not limited to monoclonal antibodies, polyclonal antibodies, multispecific antibodies (e.g., bispecific antibodies), and/or antibody fragments (preferably those fragments that exhibit the desired antigen-binding activity). An antibody described herein can be an immunoglobulin, heavy chain antibody, light chain antibody, LRR-based antibody, or other protein scaffold with antibody-like properties, as well as other immunological binding moiety known in the art, including, e.g., a Fab, Fab′, Fab′2, Fab2, Fab3, F(ab′)2, Fd, Fv, Feb, scFv, SMIP, diabody, triabody, tetrabody, minibody, Nanobody® (single domain antibody), maxibody, tandab, DVD, BiTe, TandAb, or the like, or any combination thereof. In some embodiments, the antibody is a heavy chain antibody. In some embodiments, the antibody is a camelid antibody. In some embodiments, the antibody is a llama antibody. In some embodiments, the antibody is an alpaca antibody. In some embodiments, the antibody is a mouse antibody. In some embodiments, the antibody is a human antibody. In some embodiments, the antibody is a naïve human antibody. In some embodiments, the antibody comprises a heavy chain variable region and one or more constant regions (e.g., CH2 and CH3). In some embodiments, the antibody is a Nanobody®, also referred to as a single domain antibody or “VHH.” The subunit structures and three-dimensional configurations of different classes of antibodies are known in the art.

A “monoclonal antibody” or “mAb” refers to an antibody obtained from a population of substantially homogeneous antibodies, i.e., the individual antibodies comprising the population are identical and/or bind the same epitope, except for possible variant antibodies (e.g., containing naturally occurring mutations or arising during production of a monoclonal antibody preparation), such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

An “antigen-binding fragment” refers to a portion of an intact antibody that binds the antigen to which the intact antibody binds. An antigen-binding fragment of an antibody includes any naturally occurring, enzymatically obtainable, synthetic, or genetically engineered polypeptide or glycoprotein that specifically binds an antigen to form a complex. Exemplary antibody fragments include, but are not limited to, Fv, Fab, Fab′, Fab′-SH, F(ab′)2; diabodies; linear antibodies; single-chain antibody molecules (e.g., scFv), single-domain antibody molecules (e.g., VHH or VH or VL domains only); and multispecific antibodies formed from antibody fragments. In some embodiments, the antigen-binding fragments of the antibodies described herein are scFvs. In some embodiments, the antigen-binding fragments of the antibodies described herein are VHH domains only. As with full antibody molecules, antigen-binding fragments may be mono-specific or multispecific (e.g., bispecific). A multispecific antigen-binding fragment of an antibody may comprise at least two different variable domains, wherein each variable domain is capable of specifically binding to a separate antigen or to a different epitope of the same antigen.

A “multispecific antibody” refers to an antibody comprising at least two different antigen binding domains that recognize and specifically bind to at least two different antigens. A “bispecific antibody” is a type of multispecific antibody and refers to an antibody comprising two different antigen binding domains that recognize and specifically bind to at least two different antigens.

A “different antigen” may refer to different and/or distinct proteins, polypeptides, or molecules; as well as different and/or distinct epitopes, which epitopes may be contained within one protein, polypeptide, or another molecule.

The term “epitope” refers to an antigenic determinant that interacts with a specific antigen binding site in the variable region of an antibody molecule known as a paratope. A single antigen may have more than one epitope. Thus, different antibodies may bind to different areas of an antigen and may have different biological effects. The term “epitope” also refers to a site of an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

As used herein, “selective binding,” “selectively binds,” “specific binding,” or “specifically binds” refers, with respect to an antigen binding moiety (e.g., of an antibody) and an antigen target, preferential association of an antigen binding moiety to an antigen target and not to an entity that is not the antigen target. A certain degree of non-specific binding may occur between an antigen binding moiety and a non-target. In some embodiments, an antigen binding moiety selectively binds an antigen target if binding between the antigen binding moiety and the antigen target is greater than 2-fold, greater than 5-fold, greater than 10-fold, or greater than 100-fold as compared with binding of the antigen binding moiety and a non-target. In some embodiments, an antigen binding moiety selectively binds an antigen target if the binding affinity is less than about 10⁻⁵ M, less than about 10⁻⁶ M, less than about 10⁻⁷ M, less than about 10⁻⁸ M, or less than about 10⁻⁹ M. In some embodiments, an antigen binding moiety selectively binds an epitope of an antigen target if binding between the antigen binding moiety and the epitope of the antigen target is greater than 2-fold, greater than 5-fold, greater than 10-fold, or greater than 100-fold as compared with binding of the antigen binding moiety and a non-target or another epitope of the antigen target. In some embodiments, an antigen binding moiety selectively binds an epitope of an antigen target if the binding affinity is less than about 10⁻⁵ M, less than about 10⁻⁶ M, less than about 10⁻⁷ M, less than about 10⁻⁸ M, or less than about 10⁻⁹ M.

In some embodiments, antibodies or fragments thereof that selectively bind to an identical epitope or overlapping epitope that will often cross-compete for binding to an antigen. Thus, in some embodiments, the disclosure provides an antibody or fragment thereof that cross-competes with an exemplary antibody or fragment thereof as disclosed herein. In some embodiments, to “cross-compete,” “compete,” “cross-competition,” or “competition” means antibodies or fragments thereof compete for the same epitope or binding site on a target. Such competition can be determined by an assay in which the reference antibody or fragment thereof prevents or inhibits specific binding of a test antibody or fragment thereof, and vice versa. Numerous types of competitive binding assays can be used to determine if a test molecule competes with a reference molecule for binding. Examples of assays that can be employed include solid phase direct or indirect radioimmunoassay (RIA), solid phase direct or indirect enzyme immunoassay (EIA), sandwich competition assay (see, e.g., Stahli et al. Methods in Enzymology (1983) 9:242-253), solid phase direct biotin-avidin EIA (see, e.g., Kirkland et al., J. Immunol. (1986) 137:3614-9), solid phase direct labeled assay, solid phase direct labeled sandwich assay, Luminex (Jia et al. “A novel method of Multiplexed Competitive Antibody Binning for the characterization of monoclonal antibodies” J. Immunological Methods (2004) 288, 91-98) and surface plasmon resonance (Song et al. “Epitope Mapping of Ibalizumab, a Humanized Anti-CD4 Monoclonal Antibody with Anti-HIV-1 Activity in Infected Patients” J. Virol. (2010) 84, 6935-42). In some embodiments, when a competing antibody or fragment thereof is present in excess, it will inhibit binding of a reference antibody or fragment thereof to a common antigen by at least 50%, 55%, 60%, 65%, 70%, or 75%. In some instances, binding is inhibited by at least 80%, 85%, 90%, 95%, 96%, 97%, 98%, 99%, or more.

An antibody can be an immunoglobulin molecule of four polypeptide chains, e.g., two heavy (H) chains and two light (L) chains. In some embodiments, a light chain is a lambda light chain. In some embodiments, a light chain is a kappa light chain. A heavy chain can include a heavy chain variable domain and a heavy chain constant domain. A heavy chain constant domain can include, any one or more of a CH1, hinge, CH2, CH3, and in some instances CH4 regions. A light chain can include a light chain variable domain and a light chain constant domain. A light chain constant domain can include a CL.

A heavy chain variable domain of a heavy chain and a light chain variable domain of a light chain can typically be further subdivided into regions of variability, termed complementarity determining regions (CDRs), interspersed with regions that are more conserved, termed framework regions (FR). In some embodiments, such heavy chain and/or light chain variable domains can each include three CDRs and four framework regions, arranged from amino-terminus to carboxyl-terminus in the following order: FR1, CDR1, FR2, CDR2, FR3, CDR3, FR4, one or more of which can be engineered as described herein. The CDRs in a heavy chain are designated “CDRH1,” “CDRH2,” and “CDRH3,” respectively, and the CDRs in a light chain are designated “CDRL1,” “CDRL2,” and “CDRL3.” Alternatively, the CDRs in a heavy chain are designated “HC CDR1,” “HC CDR2,” and “HC CDR3,” respectively, and the CDRs in a light chain are designated “LC CDR1,” “LC CDR2,” and “LC CDR3.”

There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

### Exemplary Anti-CLL-1 Antibodies

Provided herein are anti-CLL-1 binders, and antigen-binding fragments thereof, that bind selectively to CLL-1. In some embodiments, the anti-CLL-1 binders described herein are single chain variable fragments (scFv) or single chain antibodies.

In some embodiments, the binders are single domain antibodies. Single domain antibodies are antibodies the complementarity determining regions of which are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. According to one aspect of the disclosure, a single domain antibody as used herein is a naturally occurring single domain antibody known as heavy chain antibody devoid of light chains. Such single domain antibodies are disclosed in, e.g., International Publication No. WO 94/04678. Such variable domains derived from a heavy chain antibody naturally devoid of light chain is referred to herein as a “VHH” or “Nanobody®”. Such a VHH can be derived from antibodies raised in Camelidae species, for example in camel, dromedary, llama, vicuna, alpaca and guanaco. Other species besides Camelidae may produce heavy chain antibodies naturally devoid of light chain; such VHHs are within the scope of the disclosure. In some embodiments, the antibody is a Nanobody® or “VHH” and comprises a heavy chain variable region. In some embodiments, the antibody comprises a heavy chain variable region and one or more heavy chain constant regions. In some embodiments, the antibody comprises a heavy chain variable region and does not comprise one or more heavy chain constant regions. In some embodiments, the antibody comprises a heavy chain variable region and does not comprise a light chain region (light chain variable region or light chain constant region).

The amino acid residues of VHH domains from Camelids are numbered according to the general numbering for VH domains given by Kabat et al., “Sequence of proteins of immunological interest,” US Public Health Services, NIH (Bethesda, MD), Publication No 91-3242 (1991); see also Riechmann et al., J. Immunol. Methods (1999) 231:25-38. According to this numbering, FR1 comprises the amino acid residues at positions 1-30, CDR1 comprises the amino acid residues at positions 31-35, FR2 comprises the amino acids at positions 36-49, CDR2 comprises the amino acid residues at positions 50-65, FR3 comprises the amino acid residues at positions 66-94, CDR3 comprises the amino acid residues at positions 95-102, and FR4 comprises the amino acid residues at positions 103-113.

It should be noted, however (as is well known in the art for VH domains and for VHH domains), that the total number of amino acid residues in each of the CDRs may vary and may not correspond to the total number of amino acid residues indicated by the Kabat numbering (that is, one or more positions according to the Kabat numbering may not be occupied in an actual sequence, or the actual sequence may contain more amino acid residues than the number allowed for by the Kabat numbering). This means that, generally, the numbering according to Kabat may or may not correspond to the actual numbering of the amino acid residues in an actual sequence.

Alternative methods for numbering the amino acid residues of VH domains, which methods can also be applied in an analogous manner to VHH domains, are known in the art. However, in the present disclosure the numbering according to Kabat and applied to VHH domains as described above will be followed, unless indicated otherwise.

In some embodiments, the position numbering of an amino acid residue may be referred to based on a corresponding amino acid residue in a reference sequence.

The present disclosure provides antibodies that can include various heavy chains described herein. In some embodiments, an antibody comprises two heavy chains and light chains. In some embodiments, an antibody comprises two heavy chains, which may be two of the same heavy chain (having the same amino acid sequence) or different heavy chain (having a different amino acid sequence). In some embodiments, an antibody comprises two heavy chains, which may bind to the same epitope or a different epitope of a target antigen. In some embodiments, an antibody comprises two heavy chains, which may bind to epitopes of different target antigens. In some embodiments, the present disclosure encompasses an antibody including at least one heavy chain as disclosed herein, at least one heavy chain framework domain as disclosed herein, and/or at least one heavy chain CDR sequence as disclosed herein.

In some embodiments, an antibody disclosed herein is a homodimeric monoclonal antibody. In some embodiments, an antibody disclosed herein is a heterodimeric antibody. In some embodiments, an antibody is, e.g., a typical antibody or a diabody, triabody, tetrabody, minibody, Nanobody® (single domain antibody), maxibody, tandab, DVD, BiTe, scFv, TandAb scFv, Fab, Fab2, Fab3, F(ab′)2, or the like, or any combination thereof. In some embodiments, the antibody is a heavy chain antibody. In some embodiments, the antibody is a camelid antibody. In some embodiments, the antibody is a llama antibody. In some embodiments, the antibody is an alpaca antibody. In some embodiments, the antibody comprises a heavy chain variable region and one or more constant regions. In some embodiments, the antibody is a Nanobody®, also referred to as a single domain antibody or “VHH.” In some embodiments, the antibody comprises one, two, or three immunoglobulin constant domains (e.g., chosen from CH1, CH2, CH3, and CH4). In some embodiments, the antibody comprises one, two, or three IgG1 constant domains. In some embodiments, the antibody comprises a CH2 and a CH3 domain. In some embodiments, the antibody comprises a CH fusion. An exemplary IgG1 CH2 and CH3 domain for use in an antibody of the disclosure is provided below:

(SEQ ID NO: 1)
VVVDVSHEDPEVKFNWYVDGVEVHNAKTKPR
EEQYNSTYRVVSVLTVLHQDWLNGKEYKCKV
SNKALPAPIEKTISKAKGQPREPQVYTLPPS
RDELTKNQVSLTCLVKGFYPSDIAVEWESNG
QPENNYKTTPPVLDSDGSFFLYSKLTVDKSR
WQQGNVFSCSVMHEALHNHYTQKSLSLSPGK

In some embodiments, the antibodies are single chain antibodies.

In some embodiments, the anti-CLL-1 antibody, or antigen-binding fragment thereof, comprises an amino acid sequence of SEQ ID NOs: 15, 29, 43, 57, 71, 85, 99, 113, 127, 141, 155, 169, 309, 323, 337, 351, 365, 418, 422, 426, 430, 434, 438, 442, 446, 450, 454, 458, 462, 466, 470, 474, 478, 482, 486, 490, 494, 498, 502, 506, 510, 514, 518, 522, 526, 530, 534, 538, 542, 546, 550, 554, 558, 562, 566, 570, 574, 578, 582, 586, 590, 594, 598, 602, 606, 610, 614, 618, 622, 626, 630, 634, 638, 642, 646, 650, 654, 658, 662, 666, 670, 674, 678, 682, 686, 690, 694, 698, 702, 706, 710, 714, 718, 722, 726, 730, 734, 738, 742, 746, 750, 754, 758, 762, 766, 774, 779, 784, 788, 793, 798, 801, 804, 808, 815, 817, 819, 827, 829, 831, 833, 835, 837, 845, 847, 849, 856, 858, 860, 868, 870, 872, 879, 881, 883, 891, 893, 895, 900, 905, 910, 918, or 920.

In some embodiments, the anti-CLL-1 antibody, or antigen-binding fragment thereof, comprises an amino acid sequence of SEQ ID NOs: SEQ ID NOs: 9, 11, 13, 15, 23, 25, 27, 29, 37, 39, 41, 43, 51, 53, 55, 57, 65, 67, 69, 71, 79, 81, 83, 85, 93, 95, 97, 99, 107, 109, 111, 113, 121, 123, 125, 127, 135, 137, 139, 141, 149, 151, 153, 155, 163, 165, 167, 169, 174, 179, 184, 189, 194, 199, 204, 209, 214, 219, 224, 229, 234, 239, 244, 249, 254, 259, 264, 269, 275, 280, 285, 290, 295, 303, 305, 307, 309, 317, 319, 321, 323, 331, 333, 335, 337, 345, 347, 349, 351, 359, 361, 363, 365, 367-368, 418, 422, 426, 430, 434, 438, 442, 446, 450, 454, 458, 462, 466, 470, 474, 478, 482, 486, 490, 494, 498, 502, 506, 510, 514, 518, 522, 526, 530, 534, 538, 542, 546, 550, 554, 558, 562, 566, 570, 574, 578, 582, 586, 590, 594, 598, 602, 606, 610, 614, 618, 622, 626, 630, 634, 638, 642, 646, 650, 654, 658, 662, 666, 670, 674, 678, 682, 686, 690, 694, 698, 702, 706, 710, 714, 718, 722, 726, 730, 734, 738, 742, 746, 750, 754, 758, 762, 766, 774, 779, 784, 788, 793, 798, 801, 804, 808, 815, 817, 819, 827, 829, 831, 833, 835, 837, 845, 847, 849, 856, 858, 860, 868, 870, 872, 879, 881, 883, 891, 893, 895, 900, 905, 910, 918, 920. In some embodiments, the anti-CLL-1 antibody, or antigen-binding fragment thereof, comprises a CDR sequence encompassed within any one of SEQ ID NOs: 15, 29, 43, 57, 71, 85, 99, 113, 127, 141, 155, 169, 309, 323, 337, 351, 365, 418, 422, 426, 430, 434, 438, 442, 446, 450, 454, 458, 462, 466, 470, 474, 478, 482, 486, 490, 494, 498, 502, 506, 510, 514, 518, 522, 526, 530, 534, 538, 542, 546, 550, 554, 558, 562, 566, 570, 574, 578, 582, 586, 590, 594, 598, 602, 606, 610, 614, 618, 622, 626, 630, 634, 638, 642, 646, 650, 654, 658, 662, 666, 670, 674, 678, 682, 686, 690, 694, 698, 702, 706, 710, 714, 718, 722, 726, 730, 734, 738, 742, 746, 750, 754, 758, 762, 766, 774, 779, 784, 788, 793, 798, 801, 804, 808, 815, 817, 819, 827, 829, 831, 833, 835, 837, 845, 847, 849, 856, 858, 860, 868, 870, 872, 879, 881, 883, 891, 893, 895, 900, 905, 910, 918, or 920.

In some embodiments, the anti-CLL-1 antibody, or antigen-binding fragment thereof, comprises a CDR comprising the sequence of any one of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916o.

In some embodiments, the anti-CLL-1 antibody, or antigen-binding fragment thereof, comprises a heavy chain CDR1, CDR2, and CDR3 encompassed within any one of SEQ ID NOs:5-7, 19-21, 33-35, 47-49, 61-63, 75-77, 89-91, 103-105, 117-119, 131-133, 145-147, 159-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 299-301, 313-315, 327-329, 341-343, 355-357, 423-425, 431-433, 439-441, 447-449, 455-457, 463-465, 471-473, 479-481, 487-489, 495-497, 503-505, 511-513, 519-521, 527-529, 535-537, 543-545, 551-553, 559-561, 567-569, 575-577, 583-585, 591-593, 599-601, 607-609, 615-617, 623-625, 631-633, 639-641, 647-649, 655-657, 663-665, 671-673, 679-681, 687-689, 695-697, 703-705, 711-713, 715-746, 748-808, 811-813, 816-817, 823-825, 828-829, 834-835, 841-843, 846-847, 852-854, 857-858, 864-866, 869-870, 875-877, 880-881, 887-889, 892-893, 896-910, 914-916, or 919-920. In some embodiments, the anti-CLL-1 antibody, or antigen-binding fragment thereof, comprises a light chain CDR1, CDR2, and CDR3 encompassed within any one of SEQ ID NOs: 2-4, 16-18, 30-32, 44-46, 58-60, 72-74, 86-88, 100-102, 114-116, 128-130, 142-144, 156-158, 296-298, 310-312, 324-326, 338-340, 352-354, 419-421, 427-429, 435-437, 443-445, 451-453, 459-461, 467-469, 475-477, 483-485, 491-493, 499-501, 507-509, 515-517, 523-525, 531-533, 539-541, 547-549, 555-557, 563-565, 571-573, 579-581, 587-589, 595-597, 603-605, 611-613, 619-621, 627-629, 635-637, 643-645, 651-653, 659-661, 667-669, 675-677, 683-685, 691-693, 699-701, 707-709, 809-810, 820-822, 838-840, 850-851, 861-863, 873-874, 884-886, or 911-913.

In some embodiments, the anti-CLL-1 antibody, or antigen-binding fragment thereof, comprises a heavy chain CDR1, CDR2, and CDR3 encompassed within any one of SEQ ID NOs: 5-7, 19-21, 33-35, 47-49, 61-63, 75-77, 89-91, 103-105, 117-119, 131-133, 145-147, 159-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 299-301, 313-315, 327-329, 341-343, 355-357, 423-425, 431-433, 439-441, 447-449, 455-457, 463-465, 471-473, 479-481, 487-489, 495-497, 503-505, 511-513, 519-521, 527-529, 535-537, 543-545, 551-553, 559-561, 567-569, 575-577, 583-585, 591-593, 599-601, 607-609, 615-617, 623-625, 631-633, 639-641, 647-649, 655-657, 663-665, 671-673, 679-681, 687-689, 695-697, 703-705, 711-713, 715-746, 748-808, 811-813, 816-817, 823-825, 828-829, 834-835, 841-843, 846-847, 852-854, 857-858, 864-866, 869-870, 875-877, 880-881, 887-889, 892-893, 896-910, 914-916, or 919-920 and a light chain CDR1, CDR2, and CDR3 encompassed within any one of SEQ ID NOs: 2-4, 16-18, 30-32, 44-46, 58-60, 72-74, 86-88, 100-102, 114-116, 128-130, 142-144, 156-158, 296-298, 310-312, 324-326, 338-340, 352-354, 419-421, 427-429, 435-437, 443-445, 451-453, 459-461, 467-469, 475-477, 483-485, 491-493, 499-501, 507-509, 515-517, 523-525, 531-533, 539-541, 547-549, 555-557, 563-565, 571-573, 579-581, 587-589, 595-597, 603-605, 611-613, 619-621, 627-629, 635-637, 643-645, 651-653, 659-661, 667-669, 675-677, 683-685, 691-693, 699-701, 707-709, 809-810, 820-822, 838-840, 850-851, 861-863, 873-874, 884-886, or 911-913.

In some embodiments, the anti-CLL-1 antibody, or antigen-binding fragment thereof, comprises at least one CDR (e.g., CDR1, CDR2, and/or CDR3) of any one of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916.

In some embodiments, the anti-CLL-1 antibody, or antigen-binding fragment thereof, comprises two CDRs (e.g., CDR1, CDR2, and/or CDR3) of any one of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916.

In some embodiments, the anti-CLL-1 antibody, or antigen-binding fragment thereof, comprises three CDRs (e.g., CDR1, CDR2, and/or CDR3) of any one of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916.

In some embodiments, the anti-CLL-1 antibody, or antigen-binding fragment thereof, comprises four CDRs (e.g., CDR1, CDR2, and/or CDR3) of any one of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916.

In some embodiments, the anti-CLL-1 antibody, or antigen-binding fragment thereof, comprises five CDRs (e.g., CDR1, CDR2, and/or CDR3) of any one of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916.

In some embodiments, the anti-CLL-1 antibody, or antigen-binding fragment thereof, comprises six CDRs (e.g., CDR1, CDR2, and/or CDR3) of any one of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916.

In some embodiments, the anti-CLL-1 antibody, or antigen-binding fragment thereof, comprises at least one CDR that is at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or 100% identical to a CDR (e.g., CDR1, CDR2, and/or CDR3) of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916.

In some embodiments, the anti-CLL-1 antibody, or antigen-binding fragment thereof, comprises at least one CDR (e.g., CDR1, CDR2, and/or CDR3) depicted in any one of SEQ ID NOs: 15, 29, 43, 57, 71, 85, 99, 113, 127, 141, 155, 169, 309, 323, 337, 351, 365, 418, 422, 426, 430, 434, 438, 442, 446, 450, 454, 458, 462, 466, 470, 474, 478, 482, 486, 490, 494, 498, 502, 506, 510, 514, 518, 522, 526, 530, 534, 538, 542, 546, 550, 554, 558, 562, 566, 570, 574, 578, 582, 586, 590, 584, 598, 602, 606, 610, 614, 618, 622, 626, 630, 634, 638, 642, 646, 650, 654, 658, 662, 666, 670, 674, 678, 682, 686, 690, 694, 698, 702, 706, 710, 714, 718, 722, 726, 730, 734, 738, 742, 746, 750, 754, 758, 762, 766, 774, 779, 784, 788, 793, 798, 801, 804, 808, 815, 817, 819, 827, 829, 831, 833, 835, 837, 845, 847, 849, 856, 858, 860, 868, 870, 872, 879, 881, 883, 891, 893, 895, 900, 905, 910, 918, or 920, e.g., at least one CDR of any one of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916.

In some embodiments, the anti-CLL-1 antibody, or antigen-binding fragment thereof, comprises at least one CDR that is at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or 100% identical to a CDR (e.g., CDR1, CDR2, and/or CDR3) of any one of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916.

In some embodiments, the anti-CLL-1 antibody, or antigen-binding fragment thereof, comprises at least one CDR having one or more (e.g., 1, 2, 3, 4, 5, or more) additions, deletions, or substitutions relative to any one of the CDRs (e.g., CDR1, CDR2, and/or CDR3) provided by SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916.

The present disclosure provides, among other things, an anti-CLL-1 antibody, or antigen-binding fragment thereof comprising a VHH. In some embodiments, the anti-CLL-1 antibody, or antigen-binding fragment thereof, comprises a VHH comprising an amino acid sequence of SEQ ID NOs: 174, 179, 184, 189, 194, 199, 204, 209, 214, 219, 224, 229, 234, 239, 244, 249, 254, 259, 264, 269, 275, 280, 285, 290, 295, 714, 718, 722, 726, 730, 734, 738, 742, 746, 750, 754, 758, 762, 766 774, 779, 784, 788, 793, 798, 801, 804, 808, 817, 829, 835, 847, 858, 870, 881, 893, 900, 905, 910, or 920. In some embodiments, the anti-CLL-1 antibody, or antigen-binding fragment thereof, comprises a VHH comprising a CDR sequence provided by any one of SEQ ID NOs: 5-7, 19-21, 33-35, 47-49, 61-63, 75-77, 89-91, 103-105, 117-119, 131-133, 145-147, 159-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 299-301, 313-315, 327-329, 341-343, 355-357, 423-425, 431-433, 439-441, 447-449, 455-457, 463-465, 471-473, 479-481, 487-489, 495-497, 503-505, 511-513, 519-521, 527-529, 535-537, 543-545, 551-553, 559-561, 567-569, 575-577, 583-585, 591-593, 599-601, 607-609, 615-617, 623-625, 631-633, 639-641, 647-649, 655-657, 663-665, 671-673, 679-681, 687-689, 695-697, 703-705, 711-713, 715-717, 719-821, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916. In some embodiments, the anti-CLL-1 antibody, or antigen-binding fragment thereof, comprises a VHH comprising CDR1, CDR2, and CDR3 encompassed within any one of SEQ ID NOs: 5-7, 19-21, 33-35, 47-49, 61-63, 75-77, 89-91, 103-105, 117-119, 131-133, 145-147, 159-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 299-301, 313-315, 327-329, 341-343, 355-357, 423-425, 431-433, 439-441, 447-449, 455-457, 463-465, 471-473, 479-481, 487-489, 495-497, 503-505, 511-513, 519-521, 527-529, 535-537, 543-545, 551-553, 559-561, 567-569, 575-577, 583-585, 591-593, 599-601, 607-609, 615-617, 623-625, 631-633, 639-641, 647-649, 655-657, 663-665, 671-673, 679-681, 687-689, 695-697, 703-705, 711-713, 715-717, 719-821, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916. In some embodiments, the anti-CLL-1 antibody, or antigen-binding fragment thereof, comprises a VHH comprising at least one CDR (e.g., CDR1, CDR2, and/or CDR3) provided by any one of SEQ ID NOs: 5-7, 19-21, 33-35, 47-49, 61-63, 75-77, 89-91, 103-105, 117-119, 131-133, 145-147, 159-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 299-301, 313-315, 327-329, 341-343, 355-357, 423-425, 431-433, 439-441, 447-449, 455-457, 463-465, 471-473, 479-481, 487-489, 495-497, 503-505, 511-513, 519-521, 527-529, 535-537, 543-545, 551-553, 559-561, 567-569, 575-577, 583-585, 591-593, 599-601, 607-609, 615-617, 623-625, 631-633, 639-641, 647-649, 655-657, 663-665, 671-673, 679-681, 687-689, 695-697, 703-705, 711-713, 715-717, 719-821, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916. In some embodiments, the anti-CLL-1 antibody, or antigen-binding fragment thereof, comprises a VHH comprising at least one CDR that is at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a CDR (e.g., CDR1, CDR2, and/or CDR3) provided by any one of SEQ ID NOs: 5-7, 19-21, 33-35, 47-49, 61-63, 75-77, 89-91, 103-105, 117-119, 131-133, 145-147, 159-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 299-301, 313-315, 327-329, 341-343, 355-357, 423-425, 431-433, 439-441, 447-449, 455-457, 463-465, 471-473, 479-481, 487-489, 495-497, 503-505, 511-513, 519-521, 527-529, 535-537, 543-545, 551-553, 559-561, 567-569, 575-577, 583-585, 591-593, 599-601, 607-609, 615-617, 623-625, 631-633, 639-641, 647-649, 655-657, 663-665, 671-673, 679-681, 687-689, 695-697, 703-705, 711-713, 715-717, 719-821, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916. In some embodiments, the anti-CLL-1 antibody, or antigen-binding fragment thereof, comprises a VHH comprising at least one CDR having one or more (e.g., 1, 2, 3, 4, 5, or more) additions, deletions, or substitutions relative to any one of the CDRs (e.g., CDR1, CDR2, and/or CDR3) provided by SEQ ID NOs: 5-7, 19-21, 33-35, 47-49, 61-63, 75-77, 89-91, 103-105, 117-119, 131-133, 145-147, 159-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 299-301, 313-315, 327-329, 341-343, 355-357, 423-425, 431-433, 439-441, 447-449, 455-457, 463-465, 471-473, 479-481, 487-489, 495-497, 503-505, 511-513, 519-521, 527-529, 535-537, 543-545, 551-553, 559-561, 567-569, 575-577, 583-585, 591-593, 599-601, 607-609, 615-617, 623-625, 631-633, 639-641, 647-649, 655-657, 663-665, 671-673, 679-681, 687-689, 695-697, 703-705, 711-713, 715-717, 719-821, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916.

In some embodiments, the anti-CLL-1 antibody, or antigen-binding fragment thereof, is a monoclonal antibody, or antigen-binding fragment thereof. In some embodiments, an anti-CLL-1 antibody, or antigen-binding fragment thereof, is a humanized antibody, or antigen-binding fragment thereof. In some embodiments, the anti-CLL-1 antibody, or antigen-binding fragment thereof, is a camelid antibody or has been derived from a camelid antibody.

In some embodiments, the present disclosure provides an anti-CLL-1 antibody, or antigen-binding fragment thereof, that competes with an antibody, or antigen-binding fragment thereof, comprising an amino acid sequence of SEQ ID NOs: 2-7, 9, 11, 13, 15, 16-21, 23, 25, 27, 29, 30-35, 37, 39, 41, 43, 44-49, 51, 53, 55, 57, 58-63, 65, 67, 69, 71, 72-77, 79, 81, 83, 85, 86-91, 93, 95, 97, 99, 100-105, 107, 109, 111, 113, 114-119, 121, 123, 125, 127, 128-133, 135, 137, 139, 141, 142-147, 149, 151, 153, 155, 156-161, 163, 165, 167, 169, 170-172, 174, 175-177, 179, 180-182, 184, 185-187 189, 190-192, 194, 195-197, 199, 200-202, 204, 205-207, 209, 210-212, 214, 215-217, 219, 220-222, 224, 225-227, 229, 230-232, 234, 235-237, 239, 240-242, 244, 245-247, 249, 250-252, 254, 255-257, 259, 260-262, 264, 265-267, 269, 271-273, 275, 276-278, 280, 281-283, 285, 286-288, 290, 291-293, 295, 296-301, 303, 305, 307, 309, 310-315, 317, 319, 321, 323, 324-329, 331, 333, 335, 337, 338-343, 345, 347, 349, 351, 352-357, 359, 363, 418-772, 774-777, 779-782, 784-786, 788-791, 793-796, 798-799, 801-802, 804-806, 808-813, 815, 817, 819-825, 827, 829, 831, 833, 835, 837-843, 845, 847, 849-854, 856, 858, 860-866, 868, 870, 872-877, 879, 881, 883-889, 891, 893, 895-898, 900-903, 905-908, 910-916, 918, or 920. In some embodiments, the present disclosure provides an anti-CLL-1 antibody, or antigen-binding fragment thereof, that competes with an antibody, or antigen-binding fragment thereof, comprising an amino acid sequence of SEQ ID NOs: 15, 29, 43, 57, 71, 85, 99, 113, 127, 141, 155, 169, 309, 323, 337, 351, 365, 418, 422, 426, 430, 434, 438, 442, 446, 450, 454, 458, 462, 466, 470, 474, 478, 482, 486, 490, 494, 498, 502, 506, 510, 514, 518, 522, 526, 530, 534, 538, 542, 546, 550, 554, 558, 562, 566, 570, 574, 578, 582, 586, 590, 584, 598, 602, 606, 610, 614, 618, 622, 626, 630, 634, 638, 642, 646, 650, 654, 658, 662, 666, 670, 674, 678, 682, 686, 690, 694, 698, 702, 706, 710, 714, 718, 722, 726, 730, 734, 738, 742, 746, 750, 754, 758, 762, 766, 774, 779, 784, 788, 793, 798, 801, 804, 808, 817, 829, 831, 835, 837, 847, 849, 858, 860, 870, 872, 881, 883, 893, 895, 900, 905, 910, 920.

In some embodiments, the present disclosure provides an anti-CLL-1 antibody, or antigen-binding fragment thereof, comprising between 1 and 24 (e.g., 1, 2, 3, 4, 5, 10, or more) additions, deletions, or substitutions relative to an anti-CLL-1 antibody, or antigen-binding fragment thereof, wherein the anti-CLL-1 antibody comprises an amino acid sequence of SEQ ID NO: 15, 29, 43, 57, 71, 85, 99, 113, 127, 141, 155, 169, 309, 323, 337, 351, 365, 418, 422, 426, 430, 434, 438, 442, 446, 450, 454, 458, 462, 466, 470, 474, 478, 482, 486, 490, 494, 498, 502, 506, 510, 514, 518, 522, 526, 530, 534, 538, 542, 546, 550, 554, 558, 562, 566, 570, 574, 578, 582, 586, 590, 584, 598, 602, 606, 610, 614, 618, 622, 626, 630, 634, 638, 642, 646, 650, 654, 658, 662, 666, 670, 674, 678, 682, 686, 690, 694, 698, 702, 706, 710, 714, 718, 722, 726, 730, 734, 738, 742, 746, 750, 754, 758, 762, 766, 774, 779, 784, 788, 793, 798, 801, 804, 808, 817, 829, 831, 835, 837, 847, 849, 858, 860, 870, 872, 881, 883, 893, 895, 900, 905, 910, 920 and, e.g., the antibody or fragment selectively binds CLL-1. In some embodiments, the present disclosure provides an anti-CLL-1 antibody, or antigen-binding fragment thereof, comprising an amino acid sequence having at least 80%, 85%, 90%, 91%, 92%, 93%, 94%, 95%, 96%, 97%, 98%, or 99% sequence identity to an amino acid sequence of SEQ ID NOs: 15, 29, 43, 57, 71, 85, 99, 113, 127, 141, 155, 169, 309, 323, 337, 351, 365, 418, 422, 426, 430, 434, 438, 442, 446, 450, 454, 458, 462, 466, 470, 474, 478, 482, 486, 490, 494, 498, 502, 506, 510, 514, 518, 522, 526, 530, 534, 538, 542, 546, 550, 554, 558, 562, 566, 570, 574, 578, 582, 586, 590, 584, 598, 602, 606, 610, 614, 618, 622, 626, 630, 634, 638, 642, 646, 650, 654, 658, 662, 666, 670, 674, 678, 682, 686, 690, 694, 698, 702, 706, 710, 714, 718, 722, 726, 730, 734, 738, 742, 746, 750, 754, 758, 762, 766, 774, 779, 784, 788, 793, 798, 801, 804, 808, 817, 829, 831, 835, 837, 847, 849, 858, 860, 870, 872, 881, 883, 893, 895, 900, 905, 910, 920 and, e.g., the antibody or fragment selectively binds CLL-1.

In some embodiments, the present disclosure provides an anti-CLL-1 antibody, or antigen-binding fragment thereof, comprises a heavy chain CDR1 provided by SEQ ID NO: 5, 19, 33, 47, 61, 75, 89, 103, 117, 131, 145, 159, 170, 175, 180, 185, 190, 195, 200, 205, 210, 215, 220, 225, 230, 235, 240, 245, 250, 255, 260, 265, 271, 276, 281, 286, 291, 299, 313, 327, 341, 355, 423, 431, 439, 447, 455, 463, 471, 479, 487, 495, 503, 511, 519, 527, 535, 543, 551, 559, 567, 575, 583, 591, 599, 607, 615, 623, 631, 639, 647, 655, 663, 671, 679, 687, 695, 703, 711, 715, 719, 723, 727, 731, 735, 739, 743, 747, 751, 755, 759, 763, 767, 770, 775, 780, 785, 789, 794, 811, 823, 841, 852, 864, 875, 887, 896, 901, 906, or 914; a heavy chain CDR2 provided by SEQ ID NO: 6, 20, 34, 48, 62, 76, 90, 104, 118, 132, 146, 160, 171, 176, 181, 186, 191, 196, 201, 206, 211, 216, 221, 226, 231, 236, 241, 246, 251, 256, 261, 266, 272, 277, 282, 287, 292, 300, 314, 328, 342 356, 424, 432, 440, 448, 456, 464, 472, 480, 488, 496, 504, 512, 520, 528, 536, 544, 552, 560, 568, 576, 584, 592, 600, 608, 616, 624, 632, 640, 648, 656, 664, 672, 680, 688, 696, 704, 712, 716, 720, 724, 728, 732, 736, 740, 744, 748, 752, 756, 760, 764, 768, 771, 776, 781, 786, 790, 795, 799, 802, 805, 812, 824, 842, 853, 865, 876, 888, 897, 902, 907, or 915 and a heavy chain CDR3 provided by SEQ ID NO: 7, 21, 35, 49, 63, 77, 91, 105, 119, 133, 147, 161, 172, 177, 182, 187, 192, 197, 202, 207, 212, 217, 222, 227, 232, 237, 242, 247, 252, 257, 262, 267, 273, 278, 283, 288, 293, 301, 315, 329, 343, 357, 425, 433, 441, 449, 457, 465, 473, 481, 489, 497, 505, 513, 521, 529, 537, 545, 553, 561, 569, 577, 585, 593, 601, 609, 617, 625, 633, 641, 649, 657, 665, 673, 681, 689, 697, 705, 713, 717, 721, 725, 729, 733, 737, 741, 745, 749, 753, 757, 761, 765, 769, 772, 777, 782, 791, 796, 806, 813, 825, 843, 854, 866, 877, 889, 898, 903, 908, or 916. In some embodiments, the present disclosure provides an anti-CLL-1 antibody, or antigen-binding fragment thereof, comprises a light chain CDR1 provided by SEQ ID NO: 2, 16, 30, 44, 58, 72, 86, 100, 114, 128, 142, 156, 296, 310, 324, 338, 352, 419, 427, 435, 443, 451, 459, 467, 475, 483, 491, 499, 507, 515, 523, 531, 539, 547, 555, 563, 571, 579, 587, 595, 603, 611, 619, 627, 635, 643, 651, 659, 667, 675, 683, 691, 699, 707, 809, 820, 838, 850, 861, 873, 884, or 911, a light chain CDR2 provided by SEQ ID NO: 3, 17, 31, 45, 59, 73, 87, 101, 115, 129, 143, 157, 297, 311, 325, 339, 353, 420, 428, 436, 444, 452, 460, 468, 476, 484, 492, 500, 508, 516, 524, 532, 540, 548, 556, 564, 572, 580, 588, 596, 604, 612, 620, 628, 636, 644, 652, 660, 668, 676, 684, 692, 700, 708, 821, 839, 862, 885, or 912, and a light chain CDR3 provided by SEQ ID NO: 4, 18, 32, 46, 60, 74, 88, 102, 116, 130, 144, 158, 298, 312, 326, 340, 354, 421, 429, 437, 445, 453, 461, 469, 477, 485, 493, 501, 509, 517, 525, 533, 541, 549, 557, 565, 573, 581, 589, 597, 605, 613, 621, 629, 637, 645, 653, 661, 669, 677, 685, 693, 701, 709, 810, 822, 840, 851, 863, 874, 886, or 913.

In some embodiments, the present disclosure provides nucleic acids encoding any of the anti-CLL-1 antibodies, or antigen-binding fragments thereof, described herein. In some embodiments, the present disclosure provides nucleic acids encoding any of the anti-CLL-1 antibodies, or antigen-binding fragments thereof, comprising between 1 and 24 (e.g., 1, 2, 3, 4, 5, 10, or more) additions, deletions, or substitutions relative to an anti-CLL-1 antibody, or antigen-binding fragment thereof, wherein the anti-CLL-1 antibody comprises an amino acid sequence of SEQ ID NO: 2-7, 9, 11, 13, 15, 16-21, 23, 25, 27, 29, 30-35, 37, 39, 41, 43, 44-49, 51, 53, 55, 57, 58-63, 65, 67, 69, 71, 72-77, 79, 81, 83, 85, 86-91, 93, 95, 97, 99, 100-105, 107, 109, 111, 113, 114-119, 121, 123, 125, 127, 128-133, 135, 137, 139, 141, 142-147, 149, 151, 153, 155, 156-161, 163, 165, 167, 169, 170-172, 174, 175-177, 179, 180-182, 184, 185-187 189, 190-192, 194, 195-197, 199, 200-202, 204, 205-207, 209, 210-212, 214, 215-217, 219, 220-222, 224, 225-227, 229, 230-232, 234, 235-237, 239, 240-242, 244, 245-247, 249, 250-252, 254, 255-257, 259, 260-262, 264, 265-267, 269, 271-273, 275, 276-278, 280, 281-283, 285, 286-288, 290, 291-293, 295, 296-301, 303, 305, 307, 309, 310-315, 317, 319, 321, 323, 324-329, 331, 333, 335, 337, 338-343, 345, 347, 349, 351, 352-357, 359, 361, 363, 418-772, 774-777, 779-782, 784-786, 788-791, 793-796, 798-799, 801-802, 804-806, 808-813, 815, 817, 819-825, 827, 829, 831, 833, 835, 837-843, 845, 847, 849-854, 856, 858, 860-866, 868, 870, 872-877, 879, 881, 883-889, 891, 893, 895-898, 900-903, 905-908, 910-916, 918, or 920, and, e.g., the antibody or fragment selectively binds CLL-1. In some embodiments, the present disclosure provides nucleic acids encoding any of the anti-CLL-1 antibodies, or antigen-binding fragments thereof, comprising between 1 and 24 (e.g., 1, 2, 3, 4, 5, 10, or more) additions, deletions, or substitutions relative to an anti-CLL-1 antibody, or antigen-binding fragment thereof, wherein the anti-CLL-1 antibody comprises an amino acid sequence of SEQ ID NO: 15, 29, 43, 57, 71, 85, 99, 113, 127, 141, 155, 169, 309, 323, 337, 351, 365, 418, 422, 426, 430, 434, 438, 442, 446, 450, 454, 458, 462, 466, 470, 474, 478, 482, 486, 490, 494, 498, 502, 506, 510, 514, 518, 522, 526, 530, 534, 538, 542, 546, 550, 554, 558, 562, 566, 570, 574, 578, 582, 586, 590, 584, 598, 602, 606, 610, 614, 618, 622, 626, 630, 634, 638, 642, 646, 650, 654, 658, 662, 666, 670, 674, 678, 682, 686, 690, 694, 698, 702, 706, 710, 714, 718, 722, 726, 730, 734, 738, 742, 746, 750, 754, 758, 762, 766, 774, 779, 784, 788, 793, 798, 801, 804, 808, 817, 829, 831, 835, 837, 847, 849, 858, 860, 870, 872, 881, 883, 893, 895, 900, 905, 910, 920, and, e.g., the antibody or fragment selectively binds CLL-1. In some embodiments, the present disclosure provides nucleic acids encoding any of the anti-CLL-1 antibodies, or antigen-binding fragments thereof, comprising an amino acid sequence having at least 80%, 85%, 90%, 91%, 92%, 93%, 94%, 95%, 96%, 97%, 98%, or 99% sequence identity to an amino acid sequence of SEQ ID NOs: 2-7, 9, 11, 13, 15, 16-21, 23, 25, 27, 29, 30-35, 37, 39, 41, 43, 44-49, 51, 53, 55, 57, 58-63, 65, 67, 69, 71, 72-77, 79, 81, 83, 85, 86-91, 93, 95, 97, 99, 100-105, 107, 109, 111, 113, 114-119, 121, 123, 125, 127, 128-133, 135, 137, 139, 141, 142-147, 149, 151, 153, 155, 156-161, 163, 165, 167, 169, 170-172, 174, 175-177, 179, 180-182, 184, 185-187 189, 190-192, 194, 195-197, 199, 200-202, 204, 205-207, 209, 210-212, 214, 215-217, 219, 220-222, 224, 225-227, 229, 230-232, 234, 235-237, 239, 240-242, 244, 245-247, 249, 250-252, 254, 255-257, 259, 260-262, 264, 265-267, 269, 271-273, 275, 276-278, 280, 281-283, 285, 286-288, 290, 291-293, 295, 296-301, 303, 305, 307, 309, 310-315, 317, 319, 321, 323, 324-329, 331, 333, 335, 337, 338-343, 345, 347, 349, 351, 352-357, 359, 361, 363, 418-772, 774-777, 779-782, 784-786, 788-791, 793-796, 798-799, 801-802, 804-806, 808-813, 815, 817, 819-825, 827, 829, 831, 833, 835, 837-843, 845, 847, 849-854, 856, 858, 860-866, 868, 870, 872-877, 879, 881, 883-889, 891, 893, 895-898, 900-903, 905-908, 910-916, 918, or 920. and, e.g., the antibody or fragment selectively binds CLL-1. In some embodiments, the present disclosure provides nucleic acids encoding any of the anti-CLL-1 antibodies, or antigen-binding fragments thereof, comprising an amino acid sequence having at least 80%, 85%, 90%, 91%, 92%, 93%, 94%, 95%, 96%, 97%, 98%, or 99% sequence identity to an amino acid sequence of SEQ ID NOs: 15, 29, 43, 57, 71, 85, 99, 113, 127, 141, 155, 169, 309, 323, 337, 351, 365, 418, 422, 426, 430, 434, 438, 442, 446, 450, 454, 458, 462, 466, 470, 474, 478, 482, 486, 490, 494, 498, 502, 506, 510, 514, 518, 522, 526, 530, 534, 538, 542, 546, 550, 554, 558, 562, 566, 570, 574, 578, 582, 586, 590, 584, 598, 602, 606, 610, 614, 618, 622, 626, 630, 634, 638, 642, 646, 650, 654, 658, 662, 666, 670, 674, 678, 682, 686, 690, 694, 698, 702, 706, 710, 714, 718, 722, 726, 730, 734, 738, 742, 746, 750, 754, 758, 762, 766, 774, 779, 784, 788, 793, 798, 801, 804, 808, 817, 829, 831, 835, 837, 847, 849, 858, 860, 870, 872, 881, 883, 893, 895, 900, 905, 910, 920 and, e.g., the antibody or fragment selectively binds CLL-1.

In some embodiments, the present disclosure provides a nucleic acid having any one of the sequences provided by SEQ ID NOs: 8, 12, 14, 26, 228, 36, 40, 42, 50, 54, 56, 64, 68, 70, 78, 82, 84, 92, 96, 98, 106, 110, 112, 120, 122, 124, 126, 134, 138, 140, 148, 152, 154, 162, 166, 168, 173, 178, 183, 188, 193, 198, 203, 208, 213, 218, 223, 228, 233, 238, 243, 248, 253, 258, 263, 268, 274, 279, 284, 289, 294, 302, 306, 308, 316, 320, 322, 330, 334, 336, 344, 348, 350, 358, 362, 364, 773, 778, 783, 787, 792, 797, 800, 803, 807, 814, 816, 818, 826, 828, 830, 832, 834, 836, 844, 846, 848, 855, 857, 859, 867, 869, 871, 878, 880, 882, 890, 892, 894, 899, 904, 909, 917, 919, or 921 and encodes an antibody or antigen binding fragment thereof that binds CLL-1. In some embodiments, the present disclosure provides a nucleic acid having at least 80%, at least 85%, at least 90%, at least 91%, at least 92%, at least 93%, at least 94%, at least 95%, at least 96%, at least 97%, at least 98%, or at least 99% sequence identity to any one of the sequences provided by SEQ ID NOs: 8, 12, 14, 26, 228, 36, 40, 42, 50, 54, 56, 64, 68, 70, 78, 82, 84, 92, 96, 98, 106, 110, 112, 120, 122, 124, 126, 134, 138, 140, 148, 152, 154, 162, 166, 168, 173, 178, 183, 188, 193, 198, 203, 208, 213, 218, 223, 228, 233, 238, 243, 248, 253, 258, 263, 268, 274, 279, 284, 289, 294, 302, 306, 308, 316, 320, 322, 330, 334, 336, 344, 348, 350, 358, 362, 364, 773, 778, 783, 787, 792, 797, 800, 803, 807, 814, 816, 818, 826, 828, 830, 832, 834, 836, 844, 846, 848, 855, 857, 859, 867, 869, 871, 878, 880, 882, 890, 892, 894, 899, 904, 909, 917, 919, 921 and encodes an antibody or antigen binding fragment thereof that binds CLL-1.

The present disclosure provides, among other things, methods of making an anti-CLL-1 antibody, or antigen-binding fragment thereof. Methods of making antibodies are known in the art. For example, monoclonal antibodies can be produced using a variety of known techniques, such as the standard somatic cell hybridization technique described by Kohler and Milstein, Nature (1975) 256: 495. Other techniques for producing monoclonal antibodies also can be employed, e.g., viral or oncogenic transformation of B lymphocytes or phage display technique using libraries of human antibody genes.

In some embodiments, human antibodies are obtained by cloning the heavy and light chain genes directly from human B cells obtained from a human subject. The B cells are separated from peripheral blood (e.g., by flow cytometry, e.g., FACS), stained for B cell marker(s), and assessed for antigen binding. The RNA encoding the heavy and light chain variable regions (or the entire heavy and light chains) is extracted and reverse transcribed into DNA, from which the antibody genes are amplified (e.g., by PCR) and sequenced. The known antibody sequences can then be used to express recombinant human antibodies against a known target antigen. In some instances, human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extra-chromosomally or integrated randomly into the animal's chromosomes. In such transgenic mice, the endogenous immunoglobulin loci have generally been inactivated. Human variable regions from intact antibodies generated by such animals may be further modified, for example, by combining with a different human constant region.

In some instances, antibodies can also be made by hybridoma-based methods. In some embodiments, an animal system for generating hybridomas which produce human monoclonal antibodies is the murine system. Hybridoma production in the mouse is well known in the art, including immunization protocols and techniques for isolating and fusing immunized splenocytes. Human myeloma and mouse-human heteromyeloma cell lines for the production of human monoclonal antibodies have been described.

Human antibodies may also be generated by isolating Fv clone variable domain sequences selected from human-derived phage display libraries. Such variable domain sequences may then be combined with a desired human constant domain.

In some embodiments, the present disclosure provides methods of producing an antibody, or antigen-binding fragment thereof, comprising culturing a host cell comprising a nucleic acid encoding any of the anti-CLL-1 antibodies described herein. In some embodiments, the methods involve culturing a cell comprising a nucleic acid sequence of SEQ ID NOs: 8, 10, 12, 14, 24, 26, 228, 36, 38, 40, 42, 50, 52, 54, 56, 64, 66, 68, 70, 78, 80, 82, 84, 92, 94, 96, 98, 106, 108, 110, 112, 120, 122, 124, 126, 134, 136, 138, 140, 148, 150, 152, 154, 162, 164, 166, 168, 173, 178, 183, 188, 193, 198, 203, 208, 213, 218, 223, 228, 233, 238, 243, 248, 253, 258, 263, 268, 274, 279, 284, 289, 294, 302, 304, 306, 308, 316, 318, 320, 322, 330, 332, 334, 336, 344, 346, 348, 350, 358, 360, 362, 364, 773, 778, 783, 787, 792, 797, 800, 803, 807, 814, 816, 818, 826, 828, 830, 832, 834, 836, 844, 846, 848, 855, 857, 859, 867, 869, 871, 878, 880, 882, 890, 892, 894, 899, 904, 909, 917, 919, 921 under conditions suitable for expression of the antibody or antigen-binding fragment thereof. In some embodiments, the methods further comprise collecting, isolating, and/or purifying the antibodies or antigen-binding fragments thereof.

Sequences of exemplary antibody clones, including exemplary single chain and single domain anti-CLL-1 antibodies, are provided in Tables 1-114.

TABLE 1
Anti-CLL-1 Binder Clone 1

| Anti-CLL-1 Binder | |
|---|---|
| Clone 1 | Sequence |
| V_{L} CDR1 | GGNNIGSKSVH (SEQ ID NO: 2) |
| V_{L} CDR2 | DDSDRPS (SEQ ID NO: 3) |
| V_{L} CDR3 | QVWDSSSDHPV (SEQ ID NO: 4) |
| V_{H} CDR1 | GGTFSSY (SEQ ID NO: 5) |
| V_{H} CDR2 | IPILGI (SEQ ID NO: 6) |
| V_{H} CDR3 | AREKDDILTGAYYYGMDV (SEQ ID NO: 7) |
| V_{L} Nucleotide | TCCTATGTGCTGACTCAGCCACCCTCGGTGTCAGTGGCCCCAGGACAGACGG |
| Sequence | CCAGGATTACCTGTGGGGGAAACAACATTGGAAGTAAAAGTGTGCACTGGT |
| | ACCAGCAGAAGCCAGGCCAGGCCCCTGTGCTGGTCGTCTATGATGATAGCGA |
| | CCGGCCCTCAGGGATCCCTGAGCGATTCTCTGGCTCCAACTCTGGGAACACG |
| | GCCACCCTGACCATCAGCAGGGTCGAAGCCGGGGATGAGGCCGACTATTAC |
| | TGTCAGGTGTGGGATAGTAGTAGTGATCATCCGGTATTCGGCGGAGGAACCC |
| | AGCTGATCATTTTA (SEQ ID NO: 8) |
| V_{L} Amino Acid | SYVLTQPPSVSVAPGQTARITCGGNNIGSKSVHWYQQKPGQAPVLVVYDDSDRP |
| Sequence | SGIPERFSGSNSGNTATLTISRVEAGDEADYYCQVWDSSSDHPVFGGGTQLIIL |
| | (SEQ ID NO: 9) |
| Linker Nucleotide | GGTGGAGGCGGTTCCGGAGGCGGTGGCAGCGGCGGTGGCGGTTCG (SEQ ID |
| Sequence | NO: 10) |
| Linker Amino Acid | GGGGSGGGGSGGGGS (SEQ ID NO: 11) |
| Sequence | |
| V_{H} Nucleotide | CAGGTCCAGCTGGTACAGTCTGGGGCTGAGGTGAAGAAGCCTGGGTCCTCGG |
| Sequence | TGAAGGTCTCCTGCAAGGCTTCTGGAGGCACCTTCAGCAGCTATGCTATCAG |
| | CTGGGTGCGACAGGCCCCTGGACAAGGGCTTGAGTGGATGGGAAGGATCAT |
| | CCCTATCCTTGGTATAGCAAACTACGCACAGAAGTTCCAGGGCAGAGTCACG |
| | ATTACCGCGGACGAATCCACGAGCACAGCCTACATGGAGCTGAGCAGCCTG |
| | AGATCTGAGGACACGGCCGTGTATTACTGTGCGAGAGAGAAGGACGATATTT |
| | TGACTGGGGCCTACTACTACGGTATGGACGTCTGGGGCCAAGGGACCACGGT |
| | CACCGTCTCCTCA (SEQ ID NO: 12) |
| V_{H} Amino Acid | QVQLVQSGAEVKKPGSSVKVSCKASGGTFSSYAISWVRQAPGQGLEWMGRIIPI |
| Sequence | LGIANYAQKFQGRVTITADESTSTAYMELSSLRSEDTAVYYCAREKDDILTGAY |
| | YYGMDVWGQGTTVTVSS (SEQ ID NO: 13) |
| scFv Nucleotide | TCCTATGTGCTGACTCAGCCACCCTCGGTGTCAGTGGCCCCAGGACAGACGG |
| Sequence | CCAGGATTACCTGTGGGGGAAACAACATTGGAAGTAAAAGTGTGCACTGGT |
| | ACCAGCAGAAGCCAGGCCAGGCCCCTGTGCTGGTCGTCTATGATGATAGCGA |
| | CCGGCCCTCAGGGATCCCTGAGCGATTCTCTGGCTCCAACTCTGGGAACACG |
| | GCCACCCTGACCATCAGCAGGGTCGAAGCCGGGGATGAGGCCGACTATTACT |
| | GTCAGGTGTGGGATAGTAGTAGTGATCATCCGGTATTCGGCGGAGGAACCCA |
| | GCTGATCATTTTAGGTGGAGGCGGTTCCGGAGGCGGTGGCAGCGGCGGTGGC |
| | GGTTCGCAGGTCCAGCTGGTACAGTCTGGGGCTGAGGTGAAGAAGCCTGGGT |
| | CCTCGGTGAAGGTCTCCTGCAAGGCTTCTGGAGGCACCTTCAGCAGCTATGC |
| | TATCAGCTGGGTGCGACAGGCCCCTGGACAAGGGCTTGAGTGGATGGGAAG |
| | GATCATCCCTATCCTTGGTATAGCAAACTACGCACAGAAGTTCCAGGGCAGA |
| | GTCACGATTACCGCGGACGAATCCACGAGCACAGCCTACATGGAGCTGAGC |
| | AGCCTGAGATCTGAGGACACGGCCGTGTATTACTGTGCGAGAGAGAAGGAC |
| | GATATTTTGACTGGGGCCTACTACTACGGTATGGACGTCTGGGGCCAAGGGA |
| | CCACGGTCACCGTCTCCTCA (SEQ ID NO: 14) |
| scFv Amino Acid | SYVLTQPPSVSVAPGQTARITCGGNNIGSKSVHWYQQKPGQAPVLVVYDDSDRP |
| Sequence | SGIPERFSGSNSGNTATLTISRVEAGDEADYYCQVWDSSSDHPVFGGGTQLIILG |
| | GGGSGGGGSGGGGSQVQLVQSGAEVKKPGSSVKVSCKASGGTFSSYAISWVRQ |
| | APGQGLEWMGRIIPILGIANYAQKFQGRVTITADESTSTAYMELSSLRSEDTAVY |
| | YCAREKDDILTGAYYYGMDVWGQGTTVTVSS (SEQ ID NO: 15) |

TABLE 2
Anti-CLL-1 Binder Clone 2

| Anti-CLL-1 Binder | |
|---|---|
| Clone 2 | Sequence |
| V_{L} CDR1 | GGNNIGSKNVH (SEQ ID NO: 16) |
| V_{L} CDR2 | RDSNRPS (SEQ ID NO: 17) |
| V_{L} CDR3 | QVWDSNTYV (SEQ ID NO: 18) |
| V_{H} CDR1 | GFTFSDY (SEQ ID NO: 19) |
| V_{H} CDR2 | SSSGST (SEQ ID NO: 20) |
| V_{H} CDR3 | ARQSAEIVGAFDH (SEQ ID NO: 21) |
| V_{L} Nucleotide | CAGTCTGTGTTGACGCAGCCACTCTCAGTGTCGGTGGCCCTGGGACAGACGG |
| Sequence | CCAGGATTACCTGTGGGGGAAACAACATTGGAAGTAAAAATGTGCACTGGT |
| | ACCAGCAGAAGGCAGGCCAGGCCCCTGTGCTGGTCATCCAAAGAGATAGCA |
| | ACCGGCCCTCTGGGATCCCTGAGCGATTCTCTGGCTCCAATTCGGGGAACAC |
| | GGCCACCCTGACCATCAGTAGAGCCCAAGCCGGGGATGAGGCTGACTATTTC |
| | TGTCAGGTGTGGGACAGCAACACTTATGTCTTCGGAACTGGGACCAAGCTGA |
| | CCGTCCTA (SEQ ID NO: 22) |
| V_{L} Amino Acid | QSVLTQPLSVSVALGQTARITCGGNNIGSKNVHWYQQKAGQAPVLVIQRDSNRP |
| Sequence | SGIPERFSGSNSGNTATLTISRAQAGDEADYFCQVWDSNTYVFGTGTKLTVL |
| | (SEQ ID NO: 23) |
| Linker Nucleotide | GGTGGAGGCGGTTCCGGAGGCGGTGGCAGCGGCGGTGGCGGTTCG (SEQ ID |
| Sequence | NO: 24) |
| Linker Amino Acid | GGGGSGGGGSGGGGS (SEQ ID NO: 25) |
| Sequence | |
| V_{H} Nucleotide | GAGGTGCAGCTGGTGGAGTCTGGGGGAGGCTTGGTCAAGCCTGGAGGGTCC |
| Sequence | CTGAGACTCTCCTGTGCAGCCTCTGGATTCACCTTCAGTGACTACTACATGAG |
| | CTGGATCCGCCAGGCTCCAGGGAAGGGGCTGGAGTGGGTTTCATACATTAGT |
| | AGTAGTGGTAGTACCATATACTACGCAGACTCTGTGAAGGGCCGATTCACCA |
| | TCTCCAGGGACAACGCCAAGAACTCACTGTATCTGCAAATGAACAGCCTGAG |
| | AGACGAAGACACGGCTGTGTATTACTGTGCGAGACAATCCGCAGAGATAGT |
| | GGGAGCTTTTGACCACTGGGGCCAGGGGACAATGGTCACCGTCTCTTCA |
| | (SEQ ID NO: 26) |
| V_{H} Amino Acid | EVQLVESGGGLVKPGGSLRLSCAASGFTFSDYYMSWIRQAPGKGLEWVSYISSS |
| Sequence | GSTIYYADSVKGRFTISRDNAKNSLYLQMNSLRDEDTAVYYCARQSAEIVGAFD |
| | HWGQGTMVTVSS (SEQ ID NO: 27) |
| scFv Nucleotide | CAGTCTGTGTTGACGCAGCCACTCTCAGTGTCGGTGGCCCTGGGACAGACGG |
| Sequence | CCAGGATTACCTGTGGGGGAAACAACATTGGAAGTAAAAATGTGCACTGGT |
| | ACCAGCAGAAGGCAGGCCAGGCCCCTGTGCTGGTCATCCAAAGAGATAGCA |
| | ACCGGCCCTCTGGGATCCCTGAGCGATTCTCTGGCTCCAATTCGGGGAACAC |
| | GGCCACCCTGACCATCAGTAGAGCCCAAGCCGGGGATGAGGCTGACTATTTC |
| | TGTCAGGTGTGGGACAGCAACACTTATGTCTTCGGAACTGGGACCAAGCTGA |
| | CCGTCCTAGGTGGAGGCGGTTCCGGAGGCGGTGGCAGCGGCGGTGGCGGTT |
| | CGGAGGTGCAGCTGGTGGAGTCTGGGGGAGGCTTGGTCAAGCCTGGAGGGT |
| | CCCTGAGACTCTCCTGTGCAGCCTCTGGATTCACCTTCAGTGACTACTACATG |
| | AGCTGGATCCGCCAGGCTCCAGGGAAGGGGCTGGAGTGGGTTTCATACATTA |
| | GTAGTAGTGGTAGTACCATATACTACGCAGACTCTGTGAAGGGCCGATTCAC |
| | CATCTCCAGGGACAACGCCAAGAACTCACTGTATCTGCAAATGAACAGCCTG |
| | AGAGACGAAGACACGGCTGTGTATTACTGTGCGAGACAATCCGCAGAGATA |
| | GTGGGAGCTTTTGACCACTGGGGCCAGGGGACAATGGTCACCGTCTCTTCA |
| | (SEQ ID NO: 28) |
| scFv Amino Acid | QSVLTQPLSVSVALGQTARITCGGNNIGSKNVHWYQQKAGQAPVLVIQRDSNRP |
| Sequence | SGIPERFSGSNSGNTATLTISRAQAGDEADYFCQVWDSNTYVFGTGTKLTVLGG |
| | GGSGGGGSGGGGSEVQLVESGGGLVKPGGSLRLSCAASGFTFSDYYMSWIRQA |
| | PGKGLEWVSYISSSGSTIYYADSVKGRFTISRDNAKNSLYLQMNSLRDEDTAVY |
| | YCARQSAEIVGAFDHWGQGTMVTVSS (SEQ ID NO: 29) |

TABLE 3
Anti-CLL-1 Binder Clone 3

| Anti-CLL-1 Binder | |
|---|---|
| Clone 3 | Sequence |
| V_{L} CDR1 | TGTSSDVGGYNYVS (SEQ ID NO: 30) |
| V_{L} CDR2 | EVGKRPS (SEQ ID NO: 31) |
| V_{L} CDR3 | SSYTSSSTRV (SEQ ID NO: 32) |
| V_{H} CDR1 | GYTLTEL (SEQ ID NO: 33) |
| V_{H} CDR2 | DPEDGE (SEQ ID NO: 34) |
| V_{H} CDR3 | ATDPHMDV (SEQ ID NO: 35) |
| V_{L} Nucleotide | CAGTCTGCCCTGACTCAGCCTCCCTCCGCGTCCGGGTCTCCTGGACAGTC |
| Sequence | AGTCACCATCTCCTGCACTGGAACCAGCAGTGACGTTGGTGGTTATAACT |
| | ATGTCTCCTGGTACCAACAGCACCCAGGCAAAGCCCCCAAACTCATGAT |
| | TTATGAGGTCGGTAAGCGGCCCTCAGGGGTCCCTGATCGCTTCTCTGGCT |
| | CCAAGTCTGGCAACACGGCCTCCCTGACCGTCTCTGGGCTCCAGGCTGA |
| | GGATGAGGCTGATTATTACTGCAGCTCATATACAAGCAGCAGCACGCGG |
| | GTGTTCGGCGGAGGGACCAAGGTCACCGTCCTA (SEQ ID NO: 36) |
| V_{L} Amino Acid | QSALTQPPSASGSPGQSVTISCTGTSSDVGGYNYVSWYQQHPGKAPKL |
| Sequence | MIYEVGKRPSGVPDRFSGSKSGNTASLTVSGLQAEDEADYYCSSYTSS |
| | STRVFGGGTKVTVL (SEQ ID NO: 37) |
| Linker Nucleotide | GGTGGAGGCGGTTCCGGAGGCGGTGGCAGCGGCGGTGGCGGTTCG (SEQ |
| Sequence | ID NO: 38) |
| Linker Amino Acid | GGGGSGGGGSGGGGS (SEQ ID NO: 39) |
| Sequence | |
| V_{H} Nucleotide | GAGGTCCAGCTGGTACAGTCTGGGGCTGAGGTGAAGAAGCCTGGGGCCT |
| Sequence | CAGTGAAGGTCTCCTGCAAGGTTTCCGGATACACCCTCACTGAATTATCC |
| | ATGCACTGGGTGCGACAGGCTCCTGGAAAAGGGCTTGAGTGGATGGGAG |
| | GTTTTGATCCTGAAGATGGTGAAACAATCTACGCACAGAAGTTTCAGGG |
| | CAGAGTCACCATGACCGAGGACACATCTACAGACACAGCCTACATGGAG |
| | CTGAGCAGCCTGAGATCTGAGGACACGGCCGTGTATTATTGTGCAACAG |
| | ATCCCCACATGGACGTCTGGGGCCAAGGGACAATGGTCACCGTCTCTTCA |
| | (SEQ ID NO: 40) |
| V_{H} Amino Acid | EVQLVQSGAEVKKPGASVKVSCKVSGYTLTELSMHWVRQAPGKGLEWMG |
| Sequence | GFDPEDGETIYAQKFQGRVTMTEDTSTDTAYMELSSLRSEDTAVYYCATDP |
| | HMDVWGQGTMVTVSS (SEQ ID NO: 41) |
| scFv Nucleotide | CAGTCTGCCCTGACTCAGCCTCCCTCCGCGTCCGGGTCTCCTGGACAGTC |
| Sequence | AGTCACCATCTCCTGCACTGGAACCAGCAGTGACGTTGGTGGTTATAACT |
| | ATGTCTCCTGGTACCAACAGCACCCAGGCAAAGCCCCCAAACTCATGATT |
| | TATGAGGTCGGTAAGCGGCCCTCAGGGGTCCCTGATCGCTTCTCTGGCTC |
| | CAAGTCTGGCAACACGGCCTCCCTGACCGTCTCTGGGCTCCAGGCTGAGG |
| | ATGAGGCTGATTATTACTGCAGCTCATATACAAGCAGCAGCACGCGGGT |
| | GTTCGGCGGAGGGACCAAGGTCACCGTCCTAGGTGGAGGCGGTTCCGGA |
| | GGCGGTGGCAGCGGCGGTGGCGGTTCGGAGGTCCAGCTGGTACAGTCTG |
| | GGGCTGAGGTGAAGAAGCCTGGGGCCTCAGTGAAGGTCTCCTGCAAGGT |
| | TTCCGGATACACCCTCACTGAATTATCCATGCACTGGGTGCGACAGGCTC |
| | CTGGAAAAGGGCTTGAGTGGATGGGAGGTTTTGATCCTGAAGATGGTGA |
| | AACAATCTACGCACAGAAGTTTCAGGGCAGAGTCACCATGACCGAGGAC |
| | ACATCTACAGACACAGCCTACATGGAGCTGAGCAGCCTGAGATCTGAGG |
| | ACACGGCCGTGTATTATTGTGCAACAGATCCCCACATGGACGTCTGGGGC |
| | CAAGGGACAATGGTCACCGTCTCTTCA (SEQ ID NO: 42) |
| scFv Amino Acid | QSALTQPPSASGSPGQSVTISCTGTSSDVGGYNYVSWYQQHPGKAPKLMIYE |
| Sequence | VGKRPSGVPDRFSGSKSGNTASLTVSGLQAEDEADYYCSSYTSSSTRVFGGG |
| | TKVTVLGGGGSGGGGSGGGGSEVQLVQSGAEVKKPGASVKVSCKVSGYTL |
| | TELSMHWVRQAPGKGLEWMGGFDPEDGETIYAQKFQGRVTMTEDTSTDTA |
| | YMELSSLRSEDTAVYYCATDPHMDVWGQGTMVTVSS (SEQ ID NO: 43) |

TABLE 4
Anti-CLL-1 Binder Clone 4

| Anti-CLL-1 Binder | |
|---|---|
| Clone 4 | Sequence |
| V_{L} CDR1 | SGSSPNIGKNYVS (SEQ ID NO: 44) |
| V_{L} CDR2 | DNNKRPS (SEQ ID NO: 45) |
| V_{L} CDR3 | GTWDSSLSGYV (SEQ ID NO:46) |
| V_{H} CDR1 | GFTFSSY (SEQ ID NO: 47) |
| V_{H} CDR2 | SHDGSN (SEQ ID NO: 48) |
| V_{H} CDR3 | AREGQLTTSWDFFDY (SEQ ID NO: 49) |
| V_{L} Nucleotide | CAGTCTGTCGTGACGCAGCCGCCCTCAGTGTCTGCGGCCCCAGGACAGAAGG |
| Sequence | TCACCATCTCCTGCTCTGGAAGCAGCCCCAATATTGGCAAAAATTATGTATC |
| | CTGGTACCAGCAGCTCCCAGGAACAGCCCCCAAACTCCTCATTTATGACAAT |
| | AATAAGCGACCCTCAGGGATTCCTGACCGATTCTCTGGCTCCAAGTCTGGCA |
| | CGTCAGCCACCCTGGGCATCACCGGACTCCAGACTGGGGACGAGGCCGATT |
| | ATTACTGCGGAACATGGGATAGCAGCCTGAGTGGTTATGTCTTCGGAACTGG |
| | GACCAAGGTGACCGTCCTA (SEQ ID NO: 50) |
| V_{L} Amino Acid | QSVVTQPPSVSAAPGQKVTISCSGSSPNIGKNYVSWYQQLPGTAPKLLIYDNNKR |
| Sequence | PSGIPDRFSGSKSGTSATLGITGLQTGDEADYYCGTWDSSLSGYVFGTGTKVTVL |
| | (SEQ ID NO: 51) |
| Linker Nucleotide | GGTGGAGGCGGTTCCGGAGGCGGTGGCAGCGGCGGTGGCGGTTCG (SEQ ID |
| Sequence | NO: 52) |
| Linker Amino Acid | GGGGSGGGGSGGGGS (SEQ ID NO: 53) |
| Sequence | |
| V_{H} Nucleotide | CAGGTGCAGCTGGTGGAGTCTGGGGGAGGCGTGGTCCAGCCTGGGAGGTCC |
| Sequence | CTGAGACTCTCCTGTGCAGCCTCTGGATTCACCTTCAGTAGCTATGGCATGCA |
| | CTGGGTCCGCCAGGCTCCAGGCAAGGGGCTGGAGTGGGTGGCATTTATATCA |
| | CATGATGGAAGCAATAAATACTACGCAGACTCCGTGAAGGGCCGATCCACC |
| | ATCTCCAGAGACAATTCCAAGAGCACGCTGTTTCTGCAAATGAACAGCCTGA |
| | GAGATGAGGACACGGCTGTATATTTCTGTGCGAGAGAAGGGCAGCTCACAA |
| | CTTCTTGGGACTTCTTTGACTACTGGGGCCACGGAACCCTGGTCACTGTCTCC |
| | TCA (SEQ ID NO: 54) |
| V_{H} Amino Acid | QVQLVESGGGVVQPGRSLRLSCAASGFTFSSYGMHWVRQAPGKGLEWVAFISH |
| Sequence | DGSNKYYADSVKGRSTISRDNSKSTLFLQMNSLRDEDTAVYFCAREGQLTTSW |
| | DFFDYWGHGTLVTVSS (SEQ ID NO: 55) |
| scFv Nucleotide | CAGTCTGTCGTGACGCAGCCGCCCTCAGTGTCTGCGGCCCCAGGACAGAAGG |
| Sequence | TCACCATCTCCTGCTCTGGAAGCAGCCCCAATATTGGCAAAAATTATGTATC |
| | CTGGTACCAGCAGCTCCCAGGAACAGCCCCCAAACTCCTCATTTATGACAAT |
| | AATAAGCGACCCTCAGGGATTCCTGACCGATTCTCTGGCTCCAAGTCTGGCA |
| | CGTCAGCCACCCTGGGCATCACCGGACTCCAGACTGGGGACGAGGCCGATTA |
| | TTACTGCGGAACATGGGATAGCAGCCTGAGTGGTTATGTCTTCGGAACTGGG |
| | ACCAAGGTGACCGTCCTAGGTGGAGGCGGTTCCGGAGGCGGTGGCAGCGGC |
| | GGTGGCGGTTCGCAGGTGCAGCTGGTGGAGTCTGGGGGAGGCGTGGTCCAG |
| | CCTGGGAGGTCCCTGAGACTCTCCTGTGCAGCCTCTGGATTCACCTTCAGTAG |
| | CTATGGCATGCACTGGGTCCGCCAGGCTCCAGGCAAGGGGCTGGAGTGGGT |
| | GGCATTTATATCACATGATGGAAGCAATAAATACTACGCAGACTCCGTGAAG |
| | GGCCGATCCACCATCTCCAGAGACAATTCCAAGAGCACGCTGTTTCTGCAAA |
| | TGAACAGCCTGAGAGATGAGGACACGGCTGTATATTTCTGTGCGAGAGAAG |
| | GGCAGCTCACAACTTCTTGGGACTTCTTTGACTACTGGGGCCACGGAACCCT |
| | GGTCACTGTCTCCTCA (SEQ ID NO: 56) |
| scFv Amino Acid | QSVVTQPPSVSAAPGQKVTISCSGSSPNIGKNYVSWYQQLPGTAPKLLIYDNNKR |
| Sequence | PSGIPDRESGSKSGTSATLGITGLQTGDEADYYCGTWDSSLSGYVFGTGTKVTVL |
| | GGGGSGGGGSGGGGSQVQLVESGGGVVQPGRSLRLSCAASGFTFSSYGMHWV |
| | RQAPGKGLEWVAFISHDGSNKYYADSVKGRSTISRDNSKSTLFLQMNSLRDEDT |
| | AVYFCAREGQLTTSWDFFDYWGHGTLVTVSS (SEQ ID NO: 57) |

TABLE 5
Anti-CLL-1 Binder Clone 5

| Anti-CLL-1 Binder | |
|---|---|
| Clone 5 | Sequence |
| V_{L} CDR1 | RASQSISSYLN (SEQ ID NO: 58) |
| V_{L} CDR2 | AASSLQS (SEQ ID NO: 59) |
| V_{L} CDR3 | QQSYSTPWT (SEQ ID NO: 60) |
| V_{H} CDR1 | GFTFSSY (SEQ ID NO: 61) |
| V_{H} CDR2 | SHDGSN (SEQ ID NO: 62) |
| V_{H} CDR3 | AREGQLTTSWDFFDY (SEQ ID NO: 63) |
| V_{L} Nucleotide | GACATCCAGTTGACCCAGTCTCCATCCTCCCTGTCTGCATCTGTAGGAGACA |
| Sequence | GAGTCACCATCACTTGCCGGGCAAGCCAGAGCATTAGCAGCTATTTAAATTG |
| | GTATCAGCAGAAACCAGGGAAAGCCCCTAAGCTCCTGATCTATGCTGCATCC |
| | AGTTTGCAAAGTGGGGTCCCATCAAGGTTCAGTGGCAGTGGATCTGGGACAG |
| | ATTTCACTCTCACCATCAGCAGTCTGCAACCTGAAGATTTTGCAACTTACTAC |
| | TGTCAACAGAGTTACAGTACCCCGTGGACGTTCGGCCAAGGGACCAAGGTG |
| | GAGATCAAA (SEQ ID NO: 64) |
| V_{L} Amino Acid | DIQLTQSPSSLSASVGDRVTITCRASQSISSYLNWYQQKPGKAPKLLIYAASSLQP |
| Sequence | GVPSRFSGSGSGTDFTLTISSLQPEDFATYYCQQSYSTPWTFGQGTKVEIK (SEQ |
| | ID NO: 65) |
| Linker Nucleotide | GGTGGAGGCGGTTCCGGAGGCGGTGGCAGCGGCGGTGGCGGTTCG (SEQ ID |
| Sequence | NO: 66) |
| Linker Amino Acid | GGGGSGGGGSGGGGS (SEQ ID NO: 67) |
| Sequence | |
| V_{H} Nucleotide | CAGGTGCAGCTGGTGGAGTCTGGGGGAGGCGTGGTCCAGCCTGGGAGGTCC |
| Sequence | CTGAGACTCTCCTGTGCAGCCTCTGGATTCACCTTCAGTAGCTATGGCATGCA |
| | CTGGGTCCGCCAGGCTCCAGGCAAGGGGCTGGAGTGGGTGGCATTTATATCA |
| | CATGATGGAAGCAATAAATACTACGCAGACTCCGTGAAGGGCCGATCCACC |
| | ATCTCCAGAGACAATTCCAAGAGCACGCTGTTTCTGCAAATGAACAGCCTGA |
| | GAGATGAGGACACGGCTGTATATTTCTGTGCGAGAGAAGGGCAGCTCACAA |
| | CTTCTTGGGACTTCTTTGACTACTGGGGCCACGGAACCCTGGTCACTGTCTCC |
| | TCA (SEQ ID NO: 68) |
| V_{H} Amino Acid | QVQLVESGGGVVQPGRSLRLSCAASGFTFSSYGMHWVRQAPGKGLEWVAFISH |
| Sequence | DGSNKYYADSVKGRSTISRDNSKSTLFLQMNSLRDEDTAVYFCAREGQLTTSW |
| | DFFDYWGHGTLVTVSS (SEQ ID NO: 69) |
| scFv Nucleotide | GACATCCAGTTGACCCAGTCTCCATCCTCCCTGTCTGCATCTGTAGGAGACA |
| Sequence | GAGTCACCATCACTTGCCGGGCAAGCCAGAGCATTAGCAGCTATTTAAATTG |
| | GTATCAGCAGAAACCAGGGAAAGCCCCTAAGCTCCTGATCTATGCTGCATCC |
| | AGTTTGCAAAGTGGGGTCCCATCAAGGTTCAGTGGCAGTGGATCTGGGACAG |
| | ATTTCACTCTCACCATCAGCAGTCTGCAACCTGAAGATTTTGCAACTTACTAC |
| | TGTCAACAGAGTTACAGTACCCCGTGGACGTTCGGCCAAGGGACCAAGGTG |
| | GAGATCAAAGGTGGAGGCGGTTCCGGAGGCGGTGGCAGCGGCGGTGGCGGT |
| | TCGCAGGTGCAGCTGGTGGAGTCTGGGGGAGGCGTGGTCCAGCCTGGGAGG |
| | TCCCTGAGACTCTCCTGTGCAGCCTCTGGATTCACCTTCAGTAGCTATGGCAT |
| | GCACTGGGTCCGCCAGGCTCCAGGCAAGGGGCTGGAGTGGGTGGCATTTATA |
| | TCACATGATGGAAGCAATAAATACTACGCAGACTCCGTGAAGGGCCGATCC |
| | ACCATCTCCAGAGACAATTCCAAGAGCACGCTGTTTCTGCAAATGAACAGCC |
| | TGAGAGATGAGGACACGGCTGTATATTTCTGTGCGAGAGAAGGGCAGCTCA |
| | CAACTTCTTGGGACTTCTTTGACTACTGGGGCCACGGAACCCTGGTCACTGTC |
| | TCCTCA (SEQ ID NO: 70) |
| scFv Amino Acid | DIQLTQSPSSLSASVGDRVTITCRASQSISSYLNWYQQKPGKAPKLLIYAASSLQP |
| Sequence | GVPSRFSGSGSGTDFTLTISSLQPEDFATYYCQQSYSTPWTFGQGTKVEIKGGGG |
| | SGGGGSGGGGSQVQLVESGGGVVQPGRSLRLSCAASGFTFSSYGMHWVRQAPG |
| | KGLEWVAFISHDGSNKYYADSVKGRSTISRDNSKSTLFLQMNSLRDEDTAVYFC |
| | AREGQLTTSWDFFDYWGHGTLVTVSS (SEQ ID NO: 71) |

TABLE 6
Anti-CLL-1 Binder Clone 6

| Anti-CLL-1 Binder | |
|---|---|
| Clone 6 | Sequence |
| V_{L} CDR1 | SGGSSNIGKNYVS (SEQ ID NO: 72) |
| V_{L} CDR2 | ENQHRPS (SEQ ID NO: 73) |
| V_{L} CDR3 | ATWDDSLSAGV (SEQ ID NO: 74) |
| V_{H} CDR1 | GYSFTSY (SEQ ID NO: 75) |
| V_{H} CDR2 | YPGDSD (SEQ ID NO: 76) |
| V_{H} CDR3 | ARLGGYSGYDSEVFYLDY (SEQ ID NO: 77) |
| V_{L} Nucleotide | CAGTCTGTGTTGACGCAGCCGCCCTCAGTGTCTGCGGCCCCAGGACAGAAGG |
| Sequence | TCAGCATCTTTTGCTCTGGAGGCAGCTCCAACATTGGGAAGAACTACGTATC |
| | CTGGTACCAGCAGCTCCCAGGAACAGCCCCCAAACTCCTCATCTATGAAAAT |
| | CAGCATCGACCCTCAGGGATTCCTGACCGATTCTCTGGCTCCAAGTCTGGCA |
| | CGTCAGCCACCCTGGGCATCACCGGACTCCAGACTGGGGACGAGGCCGATT |
| | ATTACTGCGCAACATGGGATGACAGCCTGAGTGCTGGGGTGTTCGGCGGAG |
| | GGACCGAGCTGACCGTCCTA (SEQ ID NO: 78) |
| V_{L} Amino Acid | QSVLTQPPSVSAAPGQKVSIFCSGGSSNIGKNYVSWYQQLPGTAPKLLIYENQHR |
| Sequence | PSGIPDRFSGSKSGTSATLGITGLQTGDEADYYCATWDDSLSAGVFGGGTELTVL |
| | (SEQ ID NO: 79) |
| Linker Nucleotide | GGTGGAGGCGGTTCCGGAGGCGGTGGCAGCGGCGGTGGCGGTTCG (SEQ ID |
| Sequence | NO: 80) |
| Linker Amino Acid | GGGGSGGGGSGGGGS (SEQ ID NO: 81) |
| Sequence | |
| V_{H} Nucleotide | CAGCTGGTGCAGTCTGGAGCAGAGGTGAGAAAGCCCGGGGAGTCTCTGAAG |
| Sequence | ATCTCCTGTAAGGGTTCTGGATACAGCTTTACCAGCTACTGGATCGGCTGGG |
| | TGCGCCAGATGCCCGGGAAAGGCCTGGAGTGGATGGGGTTCATCTATCCTGG |
| | TGACTCTGATACCAGATACAGCCCGTCCTTCCAAGGCCAGGTCACCATCTCA |
| | GCCGACAAGTCCATCAGCACCGCCTACCTGCAGCGGAGCAGCCTGAAGGCCT |
| | CGGACACCGCCATGTATTACTGTGCGAGACTCGGTGGATATAGTGGCTACGA |
| | TTCGGAAGTCTTTTACTTAGACTACTGGGGCCAGGGAACCACGGTCACCGTC |
| | TCCTCA (SEQ ID NO: 82) |
| V_{H} Amino Acid | QLVQSGAEVRKPGESLKISCKGSGYSFTSYWIGWVRQMPGKGLEWMGFIYPGD |
| Sequence | SDTRYSPSFQGQVTISADKSISTAYLORSSLKASDTAMYYCARLGGYSGYDSE |
| | VFYLDYWGQGTTVTVSS (SEQ ID NO: 83) |
| scFv Nucleotide | CAGTCTGTGTTGACGCAGCCGCCCTCAGTGTCTGCGGCCCCAGGACAGAAGG |
| Sequence | TCAGCATCTTTTGCTCTGGAGGCAGCTCCAACATTGGGAAGAACTACGTATC |
| | CTGGTACCAGCAGCTCCCAGGAACAGCCCCCAAACTCCTCATCTATGAAAAT |
| | CAGCATCGACCCTCAGGGATTCCTGACCGATTCTCTGGCTCCAAGTCTGGCA |
| | CGTCAGCCACCCTGGGCATCACCGGACTCCAGACTGGGGACGAGGCCGATTA |
| | TTACTGCGCAACATGGGATGACAGCCTGAGTGCTGGGGTGTTCGGCGGAGGG |
| | ACCGAGCTGACCGTCCTAGGTGGAGGCGGTTCCGGAGGCGGTGGCAGCGGC |
| | GGTGGCGGTTCGCAGCTGGTGCAGTCTGGAGCAGAGGTGAGAAAGCCCGGG |
| | GAGTCTCTGAAGATCTCCTGTAAGGGTTCTGGATACAGCTTTACCAGCTACT |
| | GGATCGGCTGGGTGCGCCAGATGCCCGGGAAAGGCCTGGAGTGGATGGGGT |
| | TCATCTATCCTGGTGACTCTGATACCAGATACAGCCCGTCCTTCCAAGGCCA |
| | GGTCACCATCTCAGCCGACAAGTCCATCAGCACCGCCTACCTGCAGCGGAGC |
| | AGCCTGAAGGCCTCGGACACCGCCATGTATTACTGTGCGAGACTCGGTGGAT |
| | ATAGTGGCTACGATTCGGAAGTCTTTTACTTAGACTACTGGGGCCAGGGAAC |
| | CACGGTCACCGTCTCCTCA (SEQ ID NO: 84) |
| scFv Amino Acid | QSVLTQPPSVSAAPGQKVSIFCSGGSSNIGKNYVSWYQQLPGTAPKLLIYENQ |
| Sequence | HRPSGIPDRFSGSKSGTSATLGITGLQTGDEADYYCATWDDSLSAGVFGGGTE |
| | LTVLGGGGSGGGGSGGGGSQLVQSGAEVRKPGESLKISCKGSGYSFTSYWIGW |
| | VRQMPGKGLEWMGFIYPGDSDTRYSPSFQGQVTISADKSISTAYLQRSSLKAS |
| | DTAMYYCARLGGYSGYDSEVFYLDYWGQGTTVTVSS (SEQ ID NO: 85) |

TABLE 7
Anti-CLL-1 Binder Clone 7

| Anti-CLL-1 Binder | |
|---|---|
| Clone 7 | Sequence |
| V_{L} CDR1 | TGTSSDVGGYNYVS (SEQ ID NO: 86) |
| V_{L} CDR2 | EVGKRPS (SEQ ID NO: 87) |
| V_{L} CDR3 | SSYTSSSTRV (SEQ ID NO: 88) |
| V_{H} CDR1 | GYTLTEL (SEQ ID NO: 89) |
| V_{H} CDR2 | DPEDGE (SEQ ID NO: 90) |
| V_{H} CDR3 | ATDPHMDV (SEQ ID NO: 91) |
| V_{L} Nucleotide | CAGTCTGCCCTGACTCAGCCTCCCTCCGCGTCCGGGTCTCCTGGACAGTCAGT |
| Sequence | CACCATCTCCTGCACTGGAACCAGCAGTGACGTTGGTGGTTATAACTATGTC |
| | TCCTGGTACCAACAGCACCCAGGCAAAGCCCCCAAACTCATGATTTATGAGG |
| | TCGGTAAGCGGCCCTCAGGGGTCCCTGATCGCTTCTCTGGCTCCAAGTCTGG |
| | CAACACGGCCTCCCTGACCGTCTCTGGGCTCCAGGCTGAGGATGAGGCTGAT |
| | TATTACTGCAGCTCATATACAAGCAGCAGCACGCGGGTGTTCGGCGGAGGG |
| | ACCAAGGTCACCGTCCTA (SEQ ID NO: 92) |
| V_{L} Amino Acid | QSALTQPPSASGSPGQSVTISCTGTSSDVGGYNYVSWYQQHPGKAPKLMIYEVG |
| Sequence | KRPSGVPDRFSGSKSGNTASLTVSGLQAEDEADYYCSSYTSSSTRVFGGGTKVT |
| | VL (SEQ ID NO: 93) |
| Linker Nucleotide | GGTGGAGGCGGTTCCGGAGGCGGTGGCAGCGGCGGTGGCGGTTCG (SEQ ID |
| Sequence | NO: 94) |
| Linker Amino Acid | GGGGSGGGGSGGGGS (SEQ ID NO: 95) |
| Sequence | |
| V_{H} Nucleotide | GAGGTCCAGCTGGTACAGTCTGGGGCTGAGGTGAAGAAGCCTGGGGCCTCA |
| Sequence | GTGAAGGTCTCCTGCAAGGTTTCCGGATACACCCTCACTGAATTATCCATGC |
| | ACTGGGTGCGACAGGCTCCTGGAAAAGGGCTTGAGTGGATGGGAGGTTTTG |
| | ATCCTGAAGATGGTGAAACAATCTACGCACAGAAGTTTCAGGGCAGAGTCA |
| | CCATGACCGAGGACACATCTACAGACACAGCCTACATGGAGCTGAGCAGCC |
| | TGAGATCTGAGGACACGGCCGTGTATTATTGTGCAACAGATCCCCACATGGA |
| | CGTCTGGGGCCAAGGGACAATGGTCACCGTCTCTTCA (SEQ ID NO: 96) |
| V_{H} Amino Acid | EVQLVQSGAEVKKPGASVKVSCKVSGYTLTELSMHWVRQAPGKGLEWMGGFD |
| Sequence | PEDGETIYAQKFQGRVTMTEDTSTDTAYMELSSLRSEDTAVYYCATDPHMDVW |
| | GQGTMVTVSS (SEQ ID NO: 97) |
| scFv Nucleotide | CAGTCTGCCCTGACTCAGCCTCCCTCCGCGTCCGGGTCTCCTGGACAGTCAGT |
| Sequence | CACCATCTCCTGCACTGGAACCAGCAGTGACGTTGGTGGTTATAACTATGTC |
| | TCCTGGTACCAACAGCACCCAGGCAAAGCCCCCAAACTCATGATTTATGAGG |
| | TCGGTAAGCGGCCCTCAGGGGTCCCTGATCGCTTCTCTGGCTCCAAGTCTGG |
| | CAACACGGCCTCCCTGACCGTCTCTGGGCTCCAGGCTGAGGATGAGGCTGAT |
| | TATTACTGCAGCTCATATACAAGCAGCAGCACGCGGGTGTTCGGCGGAGGGA |
| | CCAAGGTCACCGTCCTAGGTGGAGGCGGTTCCGGAGGCGGTGGCAGCGGCG |
| | GTGGCGGTTCGGAGGTCCAGCTGGTACAGTCTGGGGCTGAGGTGAAGAAGC |
| | CTGGGGCCTCAGTGAAGGTCTCCTGCAAGGTTTCCGGATACACCCTCACTGA |
| | ATTATCCATGCACTGGGTGCGACAGGCTCCTGGAAAAGGGCTTGAGTGGATG |
| | GGAGGTTTTGATCCTGAAGATGGTGAAACAATCTACGCACAGAAGTTTCAGG |
| | GCAGAGTCACCATGACCGAGGACACATCTACAGACACAGCCTACATGGAGC |
| | TGAGCAGCCTGAGATCTGAGGACACGGCCGTGTATTATTGTGCAACAGATCC |
| | CCACATGGACGTCTGGGGCCAAGGGACAATGGTCACCGTCTCTTCA (SEQ ID |
| | NO: 98) |
| scFv Amino Acid | QSALTQPPSASGSPGQSVTISCTGTSSDVGGYNYVSWYQQHPGKAPKLMIYEVG |
| Sequence | KRPSGVPDRFSGSKSGNTASLTVSGLQAEDEADYYCSSYTSSSTRVFGGGTKVT |
| | VLGGGGSGGGGSGGGGSEVQLVQSGAEVKKPGASVKVSCKVSGYTLTELSMH |
| | WVRQAPGKGLEWMGGFDPEDGETIYAQKFQGRVTMTEDTSTDTAYMELSSLRS |
| | EDTAVYYCATDPHMDVWGQGTMVTVSS (SEQ ID NO: 99) |

TABLE 8
Anti-CLL-1 Binder Clone 8

| Anti-CLL-1 Binder | |
|---|---|
| Clone 8 | Sequence |
| V_{L} CDR1 | RASQSISSYLN (SEQ ID NO: 100) |
| V_{L} CDR2 | AASSLQS (SEQ ID NO: 101) |
| V_{L} CDR3 | QQSYSTPLT (SEQ ID NO: 102) |
| V_{H} CDR1 | GGTFTSY (SEQ ID NO: 103) |
| V_{H} CDR2 | SAYNGN (SEQ ID NO: 104) |
| V_{H} CDR3 | ALPTADDAFDI (SEQ ID NO: 105) |
| V_{L} Nucleotide | GACATCCAGATGACCCAGTCTCCATCCTCCCTGTCTGCATCTGTAGGAGACA |
| Sequence | GAGTCACCATCACTTGCCGGGCAAGTCAGAGCATTAGCAGCTATTTAAATTG |
| | GTATCAGCAGAAACCAGGGAAAGCCCCTAAGCTCCTGATCTATGCTGCATCC |
| | AGTTTGCAAAGTGGTGTCCCATCAAGGTTCAGTGGCAGTGGATCTGGGACAG |
| | ATTTCACTCTCACCATCAGCAGTCTGCAACCTGAAGATTTTGCAACTTACTAC |
| | TGTCAACAGAGTTACAGTACCCCTCTCACTTTCGGCGGAGGGACCAAGGTGG |
| | AAATCAAA (SEQ ID NO: 106) |
| V_{L} Amino Acid | DIQMTQSPSSLSASVGDRVTITCRASQSISSYLNWYQQKPGKAPKLLIYAASSLQP |
| Sequence | GVPSRFSGSGSGTDFTLTISSLQPEDFATYYCQQSYSTPLTFGGGTKVEIK (SEQ |
| | ID NO: 107) |
| Linker Nucleotide | GGTGGAGGCGGTTCCGGAGGCGGTGGCAGCGGCGGTGGCGGTTCG (SEQ ID |
| Sequence | NO: 108) |
| Linker Amino Acid | GGGGSGGGGSGGGGS (SEQ ID NO: 109) |
| Sequence | |
| V_{H} Nucleotide | CAGGTGCAGCTGGTGCAATCTGGGGCTGAGGTGAAGAAGCCTGGGTCCTCG |
| Sequence | GTGAAGGTCTCCTGCAAGGCTTCTGGAGGCACCTTCACCAGCTATGGTATCA |
| | GCTGGGTGCGACAGGCCCCTGGACAAGGGCTTGAGTGGATGGGATGGATCA |
| | GCGCTTACAATGGTAACACAAACTATGCACAGAAGCTCCAGGGCAGAGTCA |
| | CCATGACCACAGACACATCCACGAGCACAGCCTACATGGAGCTGAGGAGCC |
| | TGAGAGCCGAGGACACGGCCGTGTATTACTGTGCCTTACCTACGGCCGATGA |
| | TGCTTTTGATATCTGGGGCCAAGGGACAACGGTCACCGTCTCCTCA (SEQ ID |
| | NO: 110) |
| V_{H} Amino Acid | QVQLVQSGAEVKKPGSSVKVSCKASGGTFTSYGISWVRQAPGQGLEWMGWISA |
| Sequence | YNGNTNYAQKLQGRVTMTTDTSTSTAYMELRSLRAEDTAVYYCALPTADDAF |
| | DIWGQGTTVTVSS (SEQ ID NO: 111) |
| scFv Nucleotide | GACATCCAGATGACCCAGTCTCCATCCTCCCTGTCTGCATCTGTAGGAGACA |
| Sequence | GAGTCACCATCACTTGCCGGGCAAGTCAGAGCATTAGCAGCTATTTAAATTG |
| | GTATCAGCAGAAACCAGGGAAAGCCCCTAAGCTCCTGATCTATGCTGCATCC |
| | AGTTTGCAAAGTGGTGTCCCATCAAGGTTCAGTGGCAGTGGATCTGGGACAG |
| | ATTTCACTCTCACCATCAGCAGTCTGCAACCTGAAGATTTTGCAACTTACTAC |
| | TGTCAACAGAGTTACAGTACCCCTCTCACTTTCGGCGGAGGGACCAAGGTGG |
| | AAATCAAAGGTGGAGGCGGTTCCGGAGGCGGTGGCAGCGGCGGTGGCGGTT |
| | CGCAGGTGCAGCTGGTGCAATCTGGGGCTGAGGTGAAGAAGCCTGGGTCCTC |
| | GGTGAAGGTCTCCTGCAAGGCTTCTGGAGGCACCTTCACCAGCTATGGTATC |
| | AGCTGGGTGCGACAGGCCCCTGGACAAGGGCTTGAGTGGATGGGATGGATC |
| | AGCGCTTACAATGGTAACACAAACTATGCACAGAAGCTCCAGGGCAGAGTC |
| | ACCATGACCACAGACACATCCACGAGCACAGCCTACATGGAGCTGAGGAGC |
| | CTGAGAGCCGAGGACACGGCCGTGTATTACTGTGCCTTACCTACGGCCGATG |
| | ATGCTTTTGATATCTGGGGCCAAGGGACAACGGTCACCGTCTCCTCA (SEQ ID |
| | NO: 112) |
| scFv Amino Acid | DIQMTQSPSSLSASVGDRVTITCRASQSISSYLNWYQQKPGKAPKLLIYAASSLQS |
| Sequence | GVPSRFSGSGSGTDFTLTISSLQPEDFATYYCQQSYSTPLTFGGGTKVEIKGGGGS |
| | GGGGSGGGGSQVQLVQSGAEVKKPGSSVKVSCKASGGTFTSYGISWVRQAPGQ |
| | GLEWMGWISAYNGNTNYAQKLQGRVTMTTDTSTSTAYMELRSLRAEDTAVYY |
| | CALPTADDAFDIWGQGTTVTVSS (SEQ ID NO: 113) |

TABLE 9
Anti-CLL-1 Binder Clone 9

| Anti-CLL-1 Binder | |
|---|---|
| Clone 9 | Sequence |
| V_{L} CDR1 | GGNNIGSKSVH (SEQ ID NO: 114) |
| V_{L} CDR2 | DDSDRPS (SEQ ID NO: 115) |
| V_{L} CDR3 | QVWDSGSDHPVV (SEQ ID NO: 116) |
| V_{H} CDR1 | GFTFSSY (SEQ ID NO: 117) |
| V_{H} CDR2 | GGSGGS (SEQ ID NO: 118) |
| V_{H} CDR3 | ANIDGTF (SEQ ID NO: 119) |
| V_{L} Nucleotide | TCCTATGAGCTGACTCAGCCACCCTCGGTGTCAGTGGCCCCAGGAAAGACGG |
| Sequence | CCAGGATTACCTGTGGGGGAAACAACATTGGAAGTAAAAGTGTGCACTGGT |
| | ACCAGCAGAAGCCAGGCCAGGCCCCTGTGCTGGTCGTCTATGATGATAGCGA |
| | CCGGCCCTCAGGGATCCCTGAGCGATTCTCTGGCTCCAACTCTGGGAACACG |
| | GCCACCCTGACCATCAGCAGGGTCGAAGCCGGGGATGAGGCCGACTATTAC |
| | TGTCAGGTGTGGGATAGTGGTAGTGATCATCCCGTGGTATCCGGCGGAGGAA |
| | CCCAGCTGATCATTTTA (SEQ ID NO: 120) |
| V_{L} Amino Acid | SYELTQPPSVSVAPGKTARITCGGNNIGSKSVHWYQQKPGQAPVLVVYDDSDRP |
| Sequence | SGIPERFSGSNSGNTATLTISRVEAGDEADYYCQVWDSGSDHPVVSGGGTQLIIL |
| | (SEQ ID NO: 121) |
| Linker Nucleotide | GGTGGAGGCGGTTCCGGAGGCGGTGGCAGCGGCGGTGGCGGTTCG (SEQ ID |
| Sequence | NO: 122) |
| Linker Amino Acid | GGGGSGGGGSGGGGS (SEQ ID NO: 123) |
| Sequence | |
| V_{H} Nucleotide | CAGGTGCAGCTGGTGGAGTCTGGGGGAGGCTTGGTACAGCCTGGGGGGTCC |
| Sequence | CTGAGACTCTCCTGTGCAGCCTCTGGATTCACCTTTAGCAGCTATGCCATGAG |
| | CTGGGTCCGCCAGGCTCCAGGGAAGGGGCTGGAGTGGGTCTCAGCTATTGGT |
| | GGTAGTGGTGGTAGCACATACTACGCAGACTCCGTGAAGGGCCGGTTCACCA |
| | TCTCCAGAGACAATTCCAAGAACACGCTGTATCTGCAAATGAACAGCCTGAG |
| | AGCCGAGGACACGGCCGTATATTACTGTGCGAATATCGACGGGACCTTCTGG |
| | GGCCAGGGAACCACGGTCACCGTCTCCTCA (SEQ ID NO: 124) |
| V_{H} Amino Acid | QVQLVESGGGLVQPGGSLRLSCAASGFTFSSYAMSWVRQAPGKGLEWVSAIGG |
| Sequence | SGGSTYYADSVKGRFTISRDNSKNTLYLQMNSLRAEDTAVYYCANIDGTFWGQ |
| | GTTVTVSS (SEQ ID NO: 125) |
| scFv Nucleotide | TCCTATGAGCTGACTCAGCCACCCTCGGTGTCAGTGGCCCCAGGAAAGACGG |
| Sequence | CCAGGATTACCTGTGGGGGAAACAACATTGGAAGTAAAAGTGTGCACTGGT |
| | ACCAGCAGAAGCCAGGCCAGGCCCCTGTGCTGGTCGTCTATGATGATAGCGA |
| | CCGGCCCTCAGGGATCCCTGAGCGATTCTCTGGCTCCAACTCTGGGAACACG |
| | GCCACCCTGACCATCAGCAGGGTCGAAGCCGGGGATGAGGCCGACTATTACT |
| | GTCAGGTGTGGGATAGTGGTAGTGATCATCCCGTGGTATCCGGCGGAGGAAC |
| | CCAGCTGATCATTTTAGGTGGAGGCGGTTCCGGAGGCGGTGGCAGCGGCGGT |
| | GGCGGTTCGCAGGTGCAGCTGGTGGAGTCTGGGGGAGGCTTGGTACAGCCTG |
| | GGGGGTCCCTGAGACTCTCCTGTGCAGCCTCTGGATTCACCTTTAGCAGCTAT |
| | GCCATGAGCTGGGTCCGCCAGGCTCCAGGGAAGGGGCTGGAGTGGGTCTCA |
| | GCTATTGGTGGTAGTGGTGGTAGCACATACTACGCAGACTCCGTGAAGGGCC |
| | GGTTCACCATCTCCAGAGACAATTCCAAGAACACGCTGTATCTGCAAATGAA |
| | CAGCCTGAGAGCCGAGGACACGGCCGTATATTACTGTGCGAATATCGACGG |
| | GACCTTCTGGGGCCAGGGAACCACGGTCACCGTCTCCTCA (SEQ ID NO: 126) |
| scFv Amino Acid | SYELTQPPSVSVAPGKTARITCGGNNIGSKSVHWYQQKPGQAPVLVVYDDSDRP |
| Sequence | SGIPERFSGSNSGNTATLTISRVEAGDEADYYCQVWDSGSDHPVVSGGGTQLIIL |
| | GGGGSGGGGSGGGGSQVQLVESGGGLVQPGGSLRLSCAASGFTFSSYAMSWVR |
| | QAPGKGLEWVSAIGGSGGSTYYADSVKGRFTISRDNSKNTLYLQMNSLRAEDT |
| | AVYYCANIDGTFWGQGTTVTVSS (SEQ ID NO: 127) |

TABLE 10
Anti-CLL-1 Binder Clone 10

| Anti-CLL-1 Binder | |
|---|---|
| Clone 10 | Sequence |
| V_{L} CDR1 | RASQGISSYLA (SEQ ID NO: 128) |
| V_{L} CDR2 | SASSLQS (SEQ ID NO: 129) |
| V_{L} CDR3 | QQSYNSPT (SEQ ID NO: 130) |
| V_{H} CDR1 | GYTFTGY (SEQ ID NO: 131) |
| V_{H} CDR2 | NPNSGN (SEQ ID NO: 132) |
| V_{H} CDR3 | ARGDTGDFFDH (SEQ ID NO: 133) |
| V_{L} Nucleotide | GACATCCAGATGACCCAGTCTCCATCCTTCCTGTCTGCATCTGTAGGAGACA |
| Sequence | GAGTCACCATCACTTGCCGGGCCAGTCAGGGCATTAGCAGTTATTTAGCCTG |
| | GTATCAGCAAAAACCTGGGAAAGCCCCTAAGCTCCTGATCTATTCTGCATCC |
| | AGTTTGCAAAGTGGGGTCCCATCAAGGTTCAGTGGCAGTGGATCTGGGACAG |
| | ATTTCACTCTCACCATCAGCAGTCTGCAACCTGAAGATTTTGCAACTTACTAC |
| | TGTCAACAGAGTTACAATTCCCCTACGTTCGGCCAAGGGACACGACTGGAGA |
| | TTAAA (SEQ ID NO: 134) |
| V_{L} Amino Acid | DIQMTQSPSFLSASVGDRVTITCRASQGISSYLAWYQQKPGKAPKLLIYSASSLQS |
| Sequence | GVPSRFSGSGSGTDFTLTISSLQPEDFATYYCQQSYNSPTFGQGTRLEIK (SEQ ID |
| | NO: 135) |
| Linker Nucleotide | GGTGGAGGCGGTTCCGGAGGCGGTGGCAGCGGCGGTGGCGGTTCG (SEQ ID |
| Sequence | NO: 136) |
| Linker Amino Acid | GGGGSGGGGSGGGGS (SEQ ID NO: 137) |
| Sequence | |
| V_{H} Nucleotide | CAGGTGCAGCTGGTGCAGTCTGGGGCTGAGGTGAAGAAGCCTGGGGCCTCA |
| Sequence | GTGAAGGTCTCCTGCAAGGCTTCTGGATACACCTTCACCGGCTACTATATGC |
| | ACTGGGTGCGACAGGCCCCTGGACAAGGGCTTGAGTGGATGGGATGGATGA |
| | ACCCTAACAGTGGTAACACAGGCTATGCACAGAAGTTCCAGGGCAGAGTCA |
| | CCATGACCAGGAACACCTCCATAAGCACAGCCTACTTGGAGCTGACCAGCCT |
| | GACATCTGACGACACGGCCGTCTATTACTGTGCCAGAGGTGACACAGGCGAC |
| | TTCTTTGACCATTGGGGCCAGGGAACCCTGGTCACCGTCTCCTCA (SEQ ID |
| | NO: 138) |
| V_{H} Amino Acid | QVQLVQSGAEVKKPGASVKVSCKASGYTFTGYYMHWVRQAPGQGLEWMGW |
| Sequence | MNPNSGNTGYAQKFQGRVTMTRNTSISTAYLELTSLTSDDTAVYYCARGDTGD |
| | FFDHWGQGTLVTVSS (SEQ ID NO: 139) |
| scFv Nucleotide | GACATCCAGATGACCCAGTCTCCATCCTTCCTGTCTGCATCTGTAGGAGACA |
| Sequence | GAGTCACCATCACTTGCCGGGCCAGTCAGGGCATTAGCAGTTATTTAGCCTG |
| | GTATCAGCAAAAACCTGGGAAAGCCCCTAAGCTCCTGATCTATTCTGCATCC |
| | AGTTTGCAAAGTGGGGTCCCATCAAGGTTCAGTGGCAGTGGATCTGGGACAG |
| | ATTTCACTCTCACCATCAGCAGTCTGCAACCTGAAGATTTTGCAACTTACTAC |
| | TGTCAACAGAGTTACAATTCCCCTACGTTCGGCCAAGGGACACGACTGGAGA |
| | TTAAAGGTGGAGGCGGTTCCGGAGGCGGTGGCAGCGGCGGTGGCGGTTCGC |
| | AGGTGCAGCTGGTGCAGTCTGGGGCTGAGGTGAAGAAGCCTGGGGCCTCAG |
| | TGAAGGTCTCCTGCAAGGCTTCTGGATACACCTTCACCGGCTACTATATGCA |
| | CTGGGTGCGACAGGCCCCTGGACAAGGGCTTGAGTGGATGGGATGGATGAA |
| | CCCTAACAGTGGTAACACAGGCTATGCACAGAAGTTCCAGGGCAGAGTCAC |
| | CATGACCAGGAACACCTCCATAAGCACAGCCTACTTGGAGCTGACCAGCCTG |
| | ACATCTGACGACACGGCCGTCTATTACTGTGCCAGAGGTGACACAGGCGACT |
| | TCTTTGACCATTGGGGCCAGGGAACCCTGGTCACCGTCTCCTCA (SEQ ID NO: |
| | 140) |
| scFv Amino Acid | DIQMTQSPSFLSASVGDRVTITCRASQGISSYLAWYQQKPGKAPKLLIYSASSLQS |
| Sequence | GVPSRFSGSGSGTDFTLTISSLQPEDFATYYCQQSYNSPTFGQGTRLEIKGGGGSG |
| | GGGSGGGGSQVQLVQSGAEVKKPGASVKVSCKASGYTFTGYYMHWVRQAPG |
| | QGLEWMGWMNPNSGNTGYAQKFQGRVTMTRNTSISTAYLELTSLTSDDTAVY |
| | YCARGDTGDFFDHWGQGTLVTVSS (SEQ ID NO: 141) |

TABLE 11
Anti-CLL-1 Binder Clone 11

| Anti-CLL-1 Binder | |
|---|---|
| Clone 11 | Sequence |
| V_{L} CDR1 | TGTSSDVGASNSVS (SEQ ID NO: 142) |
| V_{L} CDR2 | DVSNRPS (SEQ ID NO: 143) |
| V_{L} CDR3 | SSYTGSSTFVV (SEQ ID NO: 144) |
| V_{H} CDR1 | GFTFSSY (SEQ ID NO: 145) |
| V_{H} CDR2 | RQDGSE (SEQ ID NO: 146) |
| V_{H} CDR3 | AREQDTAMVAFDY (SEQ ID NO: 147) |
| V_{L} Nucleotide | CAGTCTGTGTTGACTCAGCCTGCCTCCGTGTCTGGGTCTCCTGGACAGTCGAT |
| Sequence | CACCATCTCCTGCACTGGAACCAGCAGTGATGTTGGTGCTTCTAACTCTGTCT |
| | CCTGGTACCAGCGACACCCCGGCAAACCCCCCAAACTCATGATTTATGATGT |
| | CAGTAATCGGCCCTCAGGGATTTCTAATCGCTTCTCTGGCTCCAAGTCTGGCA |
| | ACACGGCCTCCCTGACCATCTCTGGGCTCCAGTCTGAGGACGAGGCTGATTA |
| | TTACTGCAGCTCATATACAGGCAGCAGCACTTTTGTGGTATTCGGCGGAGGA |
| | ACCCAGCTGATCATTTTA (SEQ ID NO: 148) |
| V_{L} Amino Acid | QSVLTQPASVSGSPGQSITISCTGTSSDVGASNSVSWYQRHPGKPPKLMIYDVSN |
| Sequence | RPSGISNRFSGSKSGNTASLTISGLQSEDEADYYCSSYTGSSTFVVFGGGTQLIIL |
| | (SEQ ID NO: 149) |
| Linker Nucleotide | GGTGGAGGCGGTTCCGGAGGCGGTGGCAGCGGCGGTGGCGGTTCG (SEQ ID |
| Sequence | NO: 150) |
| Linker Amino Acid | GGGGSGGGGSGGGGS (SEQ ID NO: 151) |
| Sequence | |
| V_{H} Nucleotide | GAGGTGCAACTGGTGGAGTCTGGGGGAGGCGTGGTCCAGCCTGGGGGGTCC |
| Sequence | CTGAGACTCTCCTGTGCAGCCTCTGGATTCACCTTTAGTAGCTATTGGATGAG |
| | CTGGGTCCGCCAGGCTCCAGGGAAGGGGCTGGAGTGGGTGGCCAACATAAG |
| | GCAAGATGGAAGTGAGAAATACTATGTGGACTCTGTGAAGGGCCGATTCAC |
| | CATCTCCAGAGACAACGCCAAGAACTCACTGTATCTGCAAATGAACAGCCTG |
| | AGAGCCGAGGACGCGGCTGTGTATTACTGTGCGAGAGAACAGGATACAGCT |
| | ATGGTCGCCTTTGACTACTGGGGCCAGGGAACCCTGGTCACCGTCTCCTCA |
| | (SEQ ID NO: 152) |
| V_{H} Amino Acid | EVQLVESGGGVVQPGGSLRLSCAASGFTFSSYWMSWVRQAPGKGLEWVANIRQ |
| Sequence | DGSEKYYVDSVKGRFTISRDNAKNSLYLQMNSLRAEDAAVYYCAREQDTAMV |
| | AFDYWGQGTLVTVSS (SEQ ID NO: 153) |
| scFv Nucleotide | CAGTCTGTGTTGACTCAGCCTGCCTCCGTGTCTGGGTCTCCTGGACAGTCGAT |
| Sequence | CACCATCTCCTGCACTGGAACCAGCAGTGATGTTGGTGCTTCTAACTCTGTCT |
| | CCTGGTACCAGCGACACCCCGGCAAACCCCCCAAACTCATGATTTATGATGT |
| | CAGTAATCGGCCCTCAGGGATTTCTAATCGCTTCTCTGGCTCCAAGTCTGGCA |
| | ACACGGCCTCCCTGACCATCTCTGGGCTCCAGTCTGAGGACGAGGCTGATTA |
| | TTACTGCAGCTCATATACAGGCAGCAGCACTTTTGTGGTATTCGGCGGAGGA |
| | ACCCAGCTGATCATTTTAGGTGGAGGCGGTTCCGGAGGCGGTGGCAGCGGCG |
| | GTGGCGGTTCGGAGGTGCAACTGGTGGAGTCTGGGGGAGGCGTGGTCCAGC |
| | CTGGGGGGTCCCTGAGACTCTCCTGTGCAGCCTCTGGATTCACCTTTAGTAGC |
| | TATTGGATGAGCTGGGTCCGCCAGGCTCCAGGGAAGGGGCTGGAGTGGGTG |
| | GCCAACATAAGGCAAGATGGAAGTGAGAAATACTATGTGGACTCTGTGAAG |
| | GGCCGATTCACCATCTCCAGAGACAACGCCAAGAACTCACTGTATCTGCAAA |
| | TGAACAGCCTGAGAGCCGAGGACGCGGCTGTGTATTACTGTGCGAGAGAAC |
| | AGGATACAGCTATGGTCGCCTTTGACTACTGGGGCCAGGGAACCCTGGTCAC |
| | CGTCTCCTCA (SEQ ID NO: 154) |
| scFv Amino Acid | QSVLTQPASVSGSPGQSITISCTGTSSDVGASNSVSWYQRHPGKPPKLMIYDVSN |
| Sequence | RPSGISNRFSGSKSGNTASLTISGLQSEDEADYYCSSYTGSSTFVVFGGGTQLIILG |
| | GGGSGGGGSGGGGSEVQLVESGGGVVQPGGSLRLSCAASGFTFSSYWMSWVR |
| | QAPGKGLEWVANIRQDGSEKYYVDSVKGRFTISRDNAKNSLYLQMNSLRAEDA |
| | AVYYCAREQDTAMVAFDYWGQGTLVTVSS (SEQ ID NO: 155) |

TABLE 12
Anti-CLL-1 Binder Clone 12

| Anti-CLL-1 Binder | |
|---|---|
| Clone 12 | Sequence |
| V_{L} CDR1 | QASQDISNYLN (SEQ ID NO: 156) |
| V_{L} CDR2 | DASNLET (SEQ ID NO: 157) |
| V_{L} CDR3 | QQANSFPFT (SEQ ID NO: 158) |
| V_{H} CDR1 | GYSFTRN (SEQ ID NO: 159) |
| V_{H} CDR2 | NPNNGD (SEQ ID NO: 160) |
| V_{H} CDR3 | ARGFWSFDV (SEQ ID NO: 161) |
| V_{L} Nucleotide | GACATCCAGATGACCCAGTCTCCATCCTCCCTGTCTGCATCTGTAGGAGACA |
| Sequence | GAGTCACCATCACTTGCCAGGCGAGTCAGGACATTAGCAACTATTTAAATTG |
| | GTATCAGCAGAAACCAGGGAAAGCCCCTAAGCTCCTGATCTACGATGCATCC |
| | AATTTGGAAACAGGGGTCCCATCAAGGTTCAGTGGAAGTGGATCTGGGACA |
| | GATTTCACTCTCACCATCAGCAGCCCGCAGCCTGAAGATCTTGCAACTTACT |
| | ATTGTCAACAGGCCAACAGTTTCCCGTTCACTTTTGGCCAGGGGACCAAGGT |
| | GGAGATCAAA (SEQ ID NO: 162) |
| V_{L} Amino Acid | DIQMTQSPSSLSASVGDRVTITCQASQDISNYLNWYQQKPGKAPKLLIYDASNLE |
| Sequence | TGVPSRFSGSGSGTDFTLTISSPQPEDLATYYCQQANSFPFTFGQGTKVEIK (SEQ |
| | ID NO: 163) |
| Linker Nucleotide | GGTGGAGGCGGTTCCGGAGGCGGTGGCAGCGGCGGTGGCGGTTCG (SEQ ID |
| Sequence | NO: 164) |
| Linker Amino Acid | GGGGSGGGGSGGGGS (SEQ ID NO: 165) |
| Sequence | |
| V_{H} Nucleotide | CAGGTGCAGCTGGTGCAGTCTGGGGCTGAGGTGAAGAAGCCTGGGTCCTCG |
| Sequence | GTGAAGGTCTCCTGCAAGGCTTCTGGATACAGCTTCACGAGAAATGATATCA |
| | CCTGGGTCCGACAGGCCAATGGACGGGGGCTTGAGTGGCTGGGACGCTTGA |
| | ACCCGAACAATGGTGACACAGTCTATGCAGAGGTCTTCCAGGGCAGAGTCGC |
| | CATGACTAGAGACACCTCCACCGACACTGCCTACATGGAGCTCGCCAGTCTT |
| | AAATCTGACGACACGGCCGTCTATTTCTGTGCGAGAGGCTTTTGGTCTTTTGA |
| | TGTCTGGGGCCAAGGGACAATGGTCACCGTCTCTTCA (SEQ ID NO: 166) |
| V_{H} Amino Acid | QVQLVQSGAEVKKPGSSVKVSCKASGYSFTRNDITWVRQANGRGLEWLGRLNP |
| Sequence | NNGDTVYAEVFQGRVAMTRDTSTDTAYMELASLKSDDTAVYFCARGFWSFDV |
| | WGQGTMVTVSS (SEQ ID NO: 167) |
| scFv Nucleotide | GACATCCAGATGACCCAGTCTCCATCCTCCCTGTCTGCATCTGTAGGAGACA |
| Sequence | GAGTCACCATCACTTGCCAGGCGAGTCAGGACATTAGCAACTATTTAAATTG |
| | GTATCAGCAGAAACCAGGGAAAGCCCCTAAGCTCCTGATCTACGATGCATCC |
| | AATTTGGAAACAGGGGTCCCATCAAGGTTCAGTGGAAGTGGATCTGGGACA |
| | GATTTCACTCTCACCATCAGCAGCCCGCAGCCTGAAGATCTTGCAACTTACT |
| | ATTGTCAACAGGCCAACAGTTTCCCGTTCACTTTTGGCCAGGGGACCAAGGT |
| | GGAGATCAAAGGTGGAGGCGGTTCCGGAGGCGGTGGCAGCGGCGGTGGCGG |
| | TTCGCAGGTGCAGCTGGTGCAGTCTGGGGCTGAGGTGAAGAAGCCTGGGTCC |
| | TCGGTGAAGGTCTCCTGCAAGGCTTCTGGATACAGCTTCACGAGAAATGATA |
| | TCACCTGGGTCCGACAGGCCAATGGACGGGGGCTTGAGTGGCTGGGACGCTT |
| | GAACCCGAACAATGGTGACACAGTCTATGCAGAGGTCTTCCAGGGCAGAGT |
| | CGCCATGACTAGAGACACCTCCACCGACACTGCCTACATGGAGCTCGCCAGT |
| | CTTAAATCTGACGACACGGCCGTCTATTTCTGTGCGAGAGGCTTTTGGTCTTT |
| | TGATGTCTGGGGCCAAGGGACAATGGTCACCGTCTCTTCA (SEQ ID NO: 168) |
| scFv Amino Acid | DIQMTQSPSSLSASVGDRVTITCQASQDISNYLNWYQQKPGKAPKLLIYDASN |
| Sequence | LETGVPSRFSGSGSGTDFTLTISSPQPEDLATYYCQQANSFPFTFGQGTKVEI |
| | KGGGGSGGGGSGGGGSQVQLVQSGAEVKKPGSSVKVSCKASGYSFTRNDITWV |
| | RQANGRGLEWLGRLNPNNGDTVYAEVFQGRVAMTRDTSTDTAYMELASLKSDD |
| | TAVYFCARGFWSFDVWGQGTMVTVSS (SEQ ID NO: 169) |

TABLE 13
Anti-CLL-1 Binder Clone 13

| Anti-CLL-1 Binder | |
|---|---|
| Clone 13 | Sequence |
| V_{H} CDR1 | SFAFSAY (SEQ ID NO: 170) |
| V_{H} CDR2 | TGSGGT (SEQ ID NO: 171) |
| V_{H} CDR3 | VSHLGGTYGSGFS (SEQ ID NO: 172) |
| V_{H} Nucleotide | CAGGTGCAGCTGGTGCAGTCTGGGGGAGGCTTGGTACAGCCTGGAGGGTCCC |
| Sequence | TGAGACTCTCCTGTGCAGCCTCTTCTTTCGCTTTCTCTGCTTATGAAATGAGC |
| | TGGGTCCGCCAGGCTCCAGGACAACGGCTTGAGTGGGTCTCAGGAATCACAG |
| | GCAGTGGTGGTACCACATATTACGAAGACTCGGTGAAGGGCCGATTCACCAT |
| | CTCCAGAGACAATTCCAAGAACACGCTGTATCTGCAAATGAACACCCTGAGA |
| | GCCGAGGACACAGCCATGTATTACTGTGTTTCTCATCTTGGTGGGACTTACG |
| | GGTCGGGCTTTTCCTGGGGCCAGGGGACCCTGGTCACCGTCTCCTCAAGT |
| | (SEQ ID NO: 173) |
| V_{H} Amino Acid | QVQLVQSGGGLVQPGGSLRLSCAASSFAFSAYEMSWVRQAPGQRLEWVSGITG |
| Sequence | SGGTTYYEDSVKGRFTISRDNSKNTLYLQMNTLRAEDTAMYYCVSHLGGTYGS |
| | GFSWGQGTLVTVSSS (SEQ ID NO: 174) |

TABLE 14
Anti-CLL-1 Binder Clone 14

| Anti-CLL-1 Binder | |
|---|---|
| Clone 14 | Sequence |
| V_{H} CDR1 | DFAFDYY (SEQ ID NO: 175) |
| V_{H} CDR2 | SGSGGT (SEQ ID NO: 176) |
| V_{H} CDR3 | ATEDPSSVY (SEQ ID NO: 177) |
| V_{H} Nucleotide | CAGGTGCAGCTGGTGCAGTCTGGGGGAGGCTTGGTACAGCCTGGAGGGTCCC |
| Sequence | TGAGACTCTCCTGTGCAGCCTCTGATTTCGCTTTCGATTATTATGAAATGAG |
| | CTGGGTCCGCCAGGCTCCAGGACAAGGGCTTGAGTGGGTCTCATCTATTAGT |
| | GGTAGTGGTGGTACCACACTCTACGCAGACTCTGTGAAGGGCCGATTCACCA |
| | TCTCCAGAGACAATTCCAAGAACACGCTGTATCTGCAAATGAATACCCTGAG |
| | AGCCGAGGACACAGCTTTGTATTACTGTGCAACAGAGGACCCTTCCAGTGTC |
| | TACTGGGGCCAGGGAACGCTGGTCACCGTCTCCTCA (SEQ ID NO: 178) |
| V_{H} Amino Acid | QVQLVQSGGGLVQPGGSLRLSCAASDFAFDYYEMSWVRQAPGQGLEWVSSISG |
| Sequence | SGGTTLYADSVKGRFTISRDNSKNTLYLQMNTLRAEDTALYYCATEDPSSVYW |
| | GQGTLVTVSS (SEQ ID NO: 179) |

TABLE 15
Anti-CLL-1 Binder Clone 15

| Anti-CLL-1 Binder | |
|---|---|
| Clone 15 | Sequence |
| V_{H} CDR1 | SFSFSDY (SEQ ID NO: 180) |
| V_{H} CDR2 | SGSGGS (SEQ ID NO: 181) |
| V_{H} CDR3 | VRVPLGHCSSTDCANPY (SEQ ID NO: 182) |
| V_{H} Nucleotide | CAGGTGCAGCTGGTGCAGTCTGGGGGAGGCTTGGTACAGCCTGGAGGGTCCCT |
| Sequence | GAGACTCTCCTGTGCAGCCTCTTCTTTCTCTTTCTCTGATTATGAAATGAGCT |
| | GGGTCCGCCAGGCTCCAGGACAACGGCTTGAGTGGGTCTCAGCTATTAGTGG |
| | TAGTGGTGGTAGCACATACTACGCAGACTCTGTGAAGGGCCGATTCACCATC |
| | TCCAGAGACAATTCCAAGAACACGCTGTATCTGCAAATGAACACCCTGAGAG |
| | CCGAGGACACAGCCACGTATTACTGTGTGAGAGTGCCCCTAGGACATTGTAG |
| | TAGTACCGACTGCGCAAATCCCTACTGGGGCCAGGGAACCCTGGTCACCGTC |
| | TCCTCA (SEQ ID NO: 183) |
| V_{H} Amino Acid | QVQLVQSGGGLVQPGGSLRLSCAASSFSFSDYEMSWVRQAPGQRLEWVSAIS |
| Sequence | GSGGSTYYADSVKGRFTISRDNSKNTLYLQMNTLRAEDTATYYCVRVPLGHC |
| | SSTDCANPYWGQGTLVTVSS (SEQ ID NO: 184) |

TABLE 16
Anti-CLL-1 Binder Clone 16

| Anti-CLL-1 Binder | |
|---|---|
| Clone 16 | Sequence |
| V_{H}CDR1 | SFDFADY |
| | (SEQ ID NO: 185) |
| V_{H}CDR2 | SGSGGD |
| | (SEQ ID NO: 186) |
| V_{H}CDR3 | AKVGPKWELVD |
| | (SEQ ID NO: 187) |
| V_{H} Nucleotide | CAGGTGCAGCTGGTGCAGTCTGGGG |
| Sequence | GAGGCTTGGTACAGCCTGGAGGGTC |
| | CCTGAGACTCTCCTGTGCAGCCTCT |
| | TCTTTCGATTTCGCTGATTATGAAA |
| | TGAGCTGGGTCCGCCAGGCTCCAGG |
| | ACAACGCCTTGAGTGGGTCTCCACT |
| | GTTTCTGGTAGCGGTGGCGACACAT |
| | GGTACGCAGACTCAGTGAAGGGCCG |
| | ATTCACCATCTCCAGAGACAATTCC |
| | AAGAACACGCTGTATCTGCAAATGA |
| | ACACCCTGAGAGCCGAGGACACGGC |
| | TGTATATTACTGTGCAAAAGTAGGG |
| | CCTAAGTGGGAGTTAGTGGACTGGG |
| | GCCAGGGAACCCTGGTCACCGTCTC |
| | CTCA |
| | (SEQ ID NO: 188) |
| V_{H} Amino Acid | QVQLVQSGGGLVQPGGSLRLSCAAS |
| Sequence | SFDFADYEMSWVRQAPGQRLEWVST |
| | VSGSGGDTWYADSVKGRFTISRDNS |
| | KNTLYLQMNTLRAEDTAVYYCAKVG |
| | PKWELVDWGQGTLVTVSS |
| | (SEQ ID NO: 189) |

TABLE 17
Anti-CLL-1 Binder Clone 17

| Anti-CLL-1 Binder | |
|---|---|
| Clone 17 | Sequence |
| V_{H}CDR1 | SFSFSDY |
| | (SEQ ID NO: 190) |
| V_{H}CDR2 | SGDGGS |
| | (SEQ ID NO: 191) |
| V_{H}CDR3 | ARDPYYYGSGRITKLDY |
| | (SEQ ID NO: 192) |
| V_{H} Nucleotide | CAGGTGCAGCTGGTGCAGTCTGGGG |
| Sequence | GAGGCTTGGTACAGCCTGGAGGGTC |
| | CCTGAGACTCTCCTGTGCAGCCTCT |
| | TCTTTCTCTTTCTCTGATTATGAAA |
| | TGAGCTGGGTCCGCCAGGCTCCAGG |
| | GAAGGGGCTGGAGTGGGTCTCTCTT |
| | ATTAGTGGGGATGGTGGTAGCACAT |
| | ACTATGCAGACTCAGTGAAGGGCCG |
| | ATTTACCATCTCCAGAGACAATTCC |
| | AAGAACACGCTGTATCTGCAAATGA |
| | ACACCCTGAGAGCCGAGGACACAGC |
| | CATGTATTACTGTGCGAGAGACCCA |
| | TATTACTATGGTTCGGGGAGAATAA |
| | CAAAACTTGACTACTGGGGCCAGGG |
| | AACCCTGGTCACCGTCTCCTCA |
| | (SEQ ID NO: 193) |
| V_{H} Amino Acid | QVQLVQSGGGLVQPGGSLRLSCAAS |
| Sequence | SFSFSDYEMSWVRQAPGKGLEWVSL |
| | ISGDGGSTYYADSVKGRFTISRDNS |
| | KNTLYLQMNTLRAEDTAMYYCARDP |
| | YYYGSGRITKLDYWGQGTLVTVSS |
| | (SEQ ID NO: 194) |

TABLE 18
Anti-CLL-1 Binder Clone 18

| Anti-CLL-1 Binder | |
|---|---|
| Clone 18 | Sequence |
| V_{H}CDR1 | GFSFSTY |
| | (SEQ ID NO: 195) |
| V_{H}CDR2 | IGNSGR |
| | (SEQ ID NO: 196) |
| V_{H}CDR3 | ARSRGGFDAFDI |
| | (SEQ ID NO: 197) |
| V_{H} Nucleotide | CAGGTGCAGCTGGTGCAGTCTGGGG |
| Sequence | GAGGCTTGGTACAGCCTGGAGGGTC |
| | CCTGAGACTCTCCTGTGCAGCCTCT |
| | GGATTCAGCTTCAGTACTTATGAAA |
| | TGAGCTGGGTCCGCCAGGCTCCAGG |
| | ACAACGGCTTGAGTGGCTCTCAGGC |
| | ATCATTGGTAATAGTGGTCGCACAT |
| | ACTACTCAGACTCCGTGAAGGGCCG |
| | ATTCACCATCTCCAGAGACAATTCC |
| | AAGAACACGCTGTATCTGCAAATGA |
| | ACACCCTGAGAGCCGAGGACACAGC |
| | CATGTATTACTGTGCACGATCTAGG |
| | GGGGGGTTTGATGCTTTTGATATCT |
| | GGGGCCAAGGGACAATGGTCACCGT |
| | CTCCTCA |
| | (SEQ ID NO: 198) |
| V_{H} Amino Acid | QVQLVQSGGGLVQPGGSLRLSCAAS |
| Sequence | GFSFSTYEMSWVRQAPGQRLEWLSG |
| | IIGNSGRTYYSDSVKGRFTISRDNS |
| | KNTLYLQMNTLRAEDTAMYYCARSR |
| | GGFDAFDIWGQGTMVTVSS |
| | (SEQ ID NO: 199) |

TABLE 19
Anti-CLL-1 Binder Clone 19

| Anti-CLL-1 Binder | |
|---|---|
| Clone 19 | Sequence |
| V_{H}CDR1 | DESFASY |
| | (SEQ ID NO: 200) |
| V_{H}CDR2 | DNGGK |
| | (SEQ ID NO: 201) |
| V_{H}CDR3 | VRDGGVAGPPT |
| | (SEQ ID NO: 202) |
| V_{H} Nucleotide | CAGGTGCAGCTGGTGCAGTCTGGGG |
| Sequence | GAGGCTTGGTACAGCCTGGAGGGTC |
| | CCTGAGACTCTCCTGTGCAGCCTCT |
| | GATTTCTCTTTCGCTTCTTATGAAA |
| | TGAGCTGGGTCCGCCAGGCTCCAGG |
| | ACAAGGGCTTGAGTGGGTCGCACTT |
| | ATTGATAACGGTGGCAAAACATATT |
| | ACGCAGACTCGGTGAAGGGCCGATT |
| | CACCATCTCCAGAGACAATTCCAAG |
| | AACACGCTGTATCTGCAAATGAACA |
| | CCCTGAGAGCCGAGGACGCGGCTTT |
| | GTATTACTGTGTGAGAGACGGGGGA |
| | GTGGCCGGTCCCCCCACCTGGGGCC |
| | AGGGAACCCTGGTCACCGTCTCCTC |
| | A |
| | (SEQ ID NO: 203) |
| V_{H} Amino Acid | QVQLVQSGGGLVQPGGSLRLSCAAS |
| Sequence | DFSFASYEMSWVRQAPGQGLEWVAL |
| | IDNGGKTYYADSVKGRFTISRDNSK |
| | NTLYLQMNTLRAEDAALYYCVRDGG |
| | VAGPPTWGQGTLVTVSS |
| | (SEQ ID NO: 204) |

TABLE 20
Anti-CLL-1 Binder Clone 20

| Anti-CLL-1 Binder | |
|---|---|
| Clone 20 | Sequence |
| V_{H}CDR1 | DFSFSYY |
| | (SEQ ID NO: 205) |
| V_{H}CDR2 | SGSGDS |
| | (SEQ ID NO: 206) |
| V_{H}CDR3 | MTDGETWGTHRYTVY |
| | (SEQ ID NO: 207) |
| V_{H} Nucleotide | CAGGTGCAGCTGGTGCAGTCTGGGG |
| Sequence | GAGGCTTGGTACAGCCTGGAGGGTC |
| | CCTGAGACTCTCCTGTGCAGCCTCT |
| | GATTTCTCTTTCTCTTATTATGAAA |
| | TGAGCTGGGTCCGCCAGGCTCCAGG |
| | GAAGGGTCTAGAGTGGGTCTCAACT |
| | ATTAGTGGTAGTGGTGATAGCACAT |
| | ACTACGCAGACTCGGTGAAGGGCCG |
| | ATTCACCATCTCCAGAGACAATACC |
| | AAGAACACGCTGTATCTGCAAATGA |
| | ACACCCTGAGAGCCGAGGACACAGC |
| | CACGTATTACTGTATGACAGATGGG |
| | GAAACTTGGGGGACTCATCGTTATA |
| | CCGTCTATTGGGGCCAGGGAGCCCT |
| | GGTCACCGTCTCCTCA |
| | (SEQ ID NO: 208) |
| V_{H} Amino Acid | QVQLVQSGGGLVQPGGSLRLSCAAS |
| Sequence | DFSFSYYEMSWVRQAPGKGLEWVST |
| | ISGSGDSTYYADSVKGRFTISRDNT |
| | KNTLYLQMNTLRAEDTATYYCMTDG |
| | ETWGTHRYTVYWGQGALVTVSS |
| | (SEQ ID NO: 209) |

TABLE 21
Anti-CLL-1 Binder Clone 21

| Anti-CLL-1 Binder | |
|---|---|
| Clone 21 | Sequence |
| V_{H}CDR1 | DFSFYFY |
| | (SEQ ID NO: 210) |
| V_{H}CDR2 | SGSGDS |
| | (SEQ ID NO: 211) |
| V_{H}CDR3 | ARDPEVGGITGEY |
| | (SEQ ID NO: 212) |
| V_{H} Nucleotide | CAGGTGCAGCTGGTGCAGTCTGGGG |
| Sequence | GAGGCTTGGTACAGCCTGGAGGGTC |
| | CCTGAGACTCTCCTGTGCAGCCTCT |
| | GATTTCTCTTTCTATTTTTATGAAA |
| | TGAGATGGGTCCGCCAGGCTCCAGG |
| | ACAAGGCCTTGAGTGGGTCTCAACT |
| | ATTAGTGGTAGTGGTGATAGCACAT |
| | ACTACGCAGACTCAGTGAAGGGCCG |
| | ATTCACCATCTCCAGAGACAATTCC |
| | AAGAACACGCTGTATCTGCAAATGA |
| | ACACCCTGAGAGCCGAGGACACGGC |
| | CGTATATTACTGTGCGAGAGATCCC |
| | GAAGTGGGAGGTATTACAGGGGAGT |
| | ACTGGGGCCAGGGCACCCTGGTCAC |
| | CGTCTCCTCA |
| | (SEQID NO: 213) |
| V_{H} Amino Acid | QVQLVQSGGGLVQPGGSLRLSCAAS |
| Sequence | DFSFYFYEMRWVRQAPGQGLEWVST |
| | ISGSGDSTYYADSVKGRFTISRDNS |
| | KNTLYLQMNTLRAEDTAVYYCARDP |
| | EVGGITGEYWGQGTLVTVSS |
| | (SEQ ID NO: 214) |

TABLE 22
Anti-CLL-1 Binder Clone 22

| Anti-CLL-1 Clone | |
|---|---|
| G2 | Sequence |
| V_{H}CDR1 | AFDFASY |
| | (SEQ ID NO: 215) |
| V_{H}CDR2 | SGSGGS |
| | (SEQ ID NO: 216) |
| V_{H}CDR3 | VKHTIRGVPEV |
| | (SEQ ID NO: 217) |
| V_{H} Nucleotide | CAGGTGCAGCTGGTGCAGTCTGGGG |
| Sequence | GAGGCTTGGTACAGCCTGGAGGGTC |
| | CCTGAGACTCTCCTGTGCAGCCTCT |
| | GCTTTCGATTTCGCTTCTTATGAAA |
| | TGAGCTGGGTCCGCCAGGCTCCAGG |
| | ACAACGCCTTGAGTGGGTCTCAGGC |
| | ATTAGTGGTAGTGGTGGTTCCACAT |
| | ACTACGCAGACTCGGTGAAGGGCCG |
| | ATTCACCATCTCCAGAGACAATTCC |
| | AAGAACACGCTGTATCTGCAAATGA |
| | ACACCCTGAGAGCCGAGGACACGGC |
| | CTTATATTACTGTGTCAAACACACG |
| | ATTCGGGGAGTTCCGGAGGTCTGGG |
| | GCCAAGGGACTATGGTCACCGTCTC |
| | CTCA |
| | (SEQ ID NO: 218) |
| V_{H} Amino Acid | QVQLVQSGGGLVQPGGSLRLSCAAS |
| Sequence | AFDFASYEMSWVRQAPGQRLEWVSG |
| | ISGSGGSTYYADSVKGRFTISRDNS |
| | KNTLYLQMNTLRAEDTALYYCVKHT |
| | IRGVPEVWGQGTMVTVSS |
| | (SEQ ID NO: 219) |

TABLE 23
Anti-CLL-1 Binder Clone 23

| Anti-CLL-1 Binder | |
|---|---|
| Clone 23 | Sequence |
| V_{H}CDR1 | YFDFDDY |
| | (SEQ ID NO: 220) |
| V_{H}CDR2 | SGGGRD |
| | (SEQ ID NO: 221) |
| V_{H}CDR3 | ARDPYYQVDDKSAYGAFDI |
| | (SEQ ID NO: 222) |
| V_{H} Nucleotide | CAGGTGCAGCTGGTGCAGTCTGGGG |
| | GAGGCTTGGTACAGCCTGGAGGGTC |
| | CCTGAGACTCTCCTGTGCAGCCTCT |
| | TATTTCGATTTCGATGATTATGAAA |
| | TGAGCTGGGTCCGCCAGGCTCCAGG |
| | GAAGGGGCTAGAGTGGGTGGCAGTT |
| | ATTAGTGGTGGTGGTCGTGACACAT |
| | GGTACGCAGACTCCGTGAAGGGCCG |
| | ATTCACCATCTCCAGAGACAATTCC |
| | AAGAACACGCTGTATCTGCAAATGA |
| Sequence | ACACCCTGAGAGCCGAGGACACAGC |
| | TGTGTATTACTGTGCGAGAGATCCC |
| | TATTACCAAGTCGATGATAAAAGTG |
| | CTTATGGGGCTTTTGATATTTGGGG |
| | CCAAGGGACAGCGGTCACCGTCTCC |
| | TCA |
| | (SEQ ID NO: 223) |
| V_{H} Amino Acid | QVQLVQSGGGLVQPGGSLRLSCAAS |
| Sequence | YFDFDDYEMSWVRQAPGKGLEWVAV |
| | ISGGGRDTWYADSVKGRFTISRDNS |
| | KNTLYLQMNTLRAEDTAVYYCARDP |
| | YYQVDDKSAYGAFDIWGQGTAVTVS |
| | S |
| | (SEQ ID NO: 224) |

TABLE 24
Anti-CLL-1 Binder Clone 24

| Anti-CLL-1 Binder | |
|---|---|
| Clone 24 | Sequence |
| V_{H}CDR1 | AFDFYDY |
| | (SEQ ID NO: 225) |
| V_{H}CDR2 | SNSGDA |
| | (SEQ ID NO: 226) |
| V_{H}CDR3 | TTIEDTLPS |
| | (SEQ ID NO: 227) |
| V_{H} Nucleotide | CAGGTGCAGCTGGTGCAGTCTGGGG |
| Sequence | GAGGCTTGGTACAGCCTGGAGGGTC |
| | CCTGAGACTCTCCTGTGCAGCCTCT |
| | GCTTTCGATTTCTATGATTATGAAA |
| | TGAGCTGGGTCCGCCAGGCTCCAGG |
| | ACAAGGGCTTGAGTGGGTCTCGGCT |
| | ATTAGTAATAGTGGTGATGCCACAT |
| | ACTACGCAGACTCAGTGAAGGGCCG |
| | ATTCACCATCTCCAGAGACAATTCC |
| | AAGAACACGCTGTATCTGCAAATGA |
| | ACACCCTGAGAGCCGAGGACACAGC |
| | CGTATATTACTGTACCACAATAGAG |
| | GATACATTACCCTCCTGGGGCCAGG |
| | GAACCCTGGTCACCGTCTCCTCA |
| | (SEQ ID NO: 228) |
| V_{H} Amino Acid | QVQLVQSGGGLVQPGGSLRLSCAAS |
| Sequence | AFDFYDYEMSWVRQAPGQGLEWVSA |
| | ISNSGDATYYADSVKGRFTISRDNS |
| | KNTLYLQMNTLRAEDTAVYYCTTIE |
| | DTLPSWGQGTLVTVSS |
| | (SEQ ID NO: 229) |

TABLE 25
Anti-CLL-1 Binder Clone 25

| Anti-CLL-1 Binder | |
|---|---|
| Clone 25 | Sequence |
| V_{H}CDR1 | SFDFSSY |
| | (SEQ ID NO: 230) |
| V_{H}CDR2 | SGSGDS |
| | (SEQ ID NO: 231) |
| V_{H}CDR3 | ARDPYYGGEYYFDY |
| | (SEQ ID NO: 232) |
| V_{H} Nucleotide | CAGGTGCAGCTGGTGCAGTCTGGGG |
| Sequence | GAGGCTTGGTACAGCCTGGAGGGTC |
| | CCTGAGACTCTCCTGTGCAGCCTCT |
| | TCTTTCGATTTCTCTTCTTATGAAA |
| | TGAGCTGGGTCCGCCAGGCTCCAGG |
| | ACAAGGGCTTGAGTGGGTCTCTCTT |
| | ATTAGTGGTAGTGGTGATAGCACAT |
| | ACTACGCAGACTCCGTGAAGGGCCG |
| | ATTCACCATCTCCAGAGACAATTCC |
| | AAGAACACGCTGTATCTGCAAATGA |
| | ACACCCTGAGAGCCGAGGACACGGC |
| | TTTGTATTACTGTGCAAGAGACCCC |
| | TACTACGGTGGCGAATATTACTTTG |
| | ACTACTGGGGCCAGGGAACCCTGGT |
| | CACCGTCTCCTCA |
| | (SEQ ID NO: 233) |
| V_{H} Amino Acid | QVQLVQSGGGLVQPGGSLRLSCAAS |
| Sequence | SFDFSSYEMSWVRQAPGQGLEWVSL |
| | ISGSGDSTYYADSVKGRFTISRDNS |
| | KNTLYLQMNTLRAEDTALYYCARDP |
| | YYGGEYYFDYWGQGTLVTVSS |
| | (SEQ ID NO: 234) |

TABLE 26
Anti-CLL-1 Binder Clone 26

| Anti-CLL-1 Binder | |
|---|---|
| Clone 26 | Sequence |
| V_{H}CDR1 | AFSFSSY |
| | (SEQ ID NO: 235) |
| V_{H}CDR2 | NGSGGS |
| | (SEQ ID NO: 236) |
| V_{H}CDR3 | ASSYSSSSQFEY |
| | (SEQ ID NO: 237) |
| V_{H} Nucleotide | CAGGTGCAGCTGGTGCAGTCTGGGG |
| Sequence | GAGGCTTGGTACAGCCTGGAGGGTC |
| | CCTGAGACTCTCCTGTGCAGCCTCT |
| | GCTTTCTCTTTCTCTTCTTATGAAA |
| | TGAGCTGGGTCCGCCAGGCTCCAGG |
| | ACAACGGCTTGAGTGGGTCGCACAT |
| | ATTAATGGTAGTGGTGGTAGCACAT |
| | ACTACGCAGACTCGGTGAAGGGCCG |
| | ATTCACCATCTCCAGAGACAATTCC |
| | AAGAACACGCTGTATCTGCAAATGA |
| | ACACCCTGAGAGCCGAGGACACAGC |
| | CACGTATTACTGTGCAAGTTCGTAT |
| | AGCAGCTCGTCGCAATTTGAGTACT |
| | GGGGCCAGGGAACCCTGGTCACCGT |
| | CTCCTCA |
| | (SEQ ID NO: 238) |
| V_{H} Amino Acid | QVQLVQSGGGLVQPGGSLRLSCAAS |
| Sequence | AFSFSSYEMSWVRQAPGQRLEWVAH |
| | INGSGGSTYYADSVKGRFTISRDNS |
| | KNTLYLQMNTLRAEDTATYYCASSY |
| | SSSSQFEYWGQGTLVTVSS |
| | (SEQ ID NO: 239) |

TABLE 27
Anti-CLL-1 Binder Clone 27

| Anti-CLL-1 Binder | |
|---|---|
| Clone 27 | Sequence |
| V_{H}CDR1 | DFSFADY |
| | (SEQ ID NO: 240) |
| V_{H}CDR2 | SGDGGS |
| | (SEQ ID NO: 241) |
| V_{H}CDR3 | ARDPDSGSYWGGDY |
| | (SEQ ID NO: 242) |
| V_{H} Nucleotide | CAGGTGCAGCTGGTGCAGTCTGGGG |
| Sequence | GAGGCTTGGTACAGCCTGGAGGGTC |
| | CCTGAGACTCTCCTGTGCAGCCTCT |
| | GATTTCTCTTTCGCTGATTATGAAA |
| | TGAGCTGGGTCCGCCAGGCTCCAGG |
| | ACAAGGCCTTGAGTGGGTCTCTCTT |
| | ATTAGTGGGGATGGTGGTAGCACAT |
| | ACTATGCAGACTCGGTGAAGGGCCG |
| | ATTCACCATCTCCAGAGACAATTCC |
| | AAGAACACGCTGTATCTGCAAATGA |
| | ACACCCTGAGAGCCGAGGACACAGC |
| | CGTATATTACTGTGCGAGAGATCCC |
| | GATAGTGGGAGCTACTGGGGGGGTG |
| | ACTACTGGGGCCAGGGAACCCTGGT |
| | CACCGTCTCCTCA |
| | (SEQ ID NO: 243) |
| V_{H} Amino Acid | QVQLVQSGGGLVQPGGSLRLSCAAS |
| Sequence | DFSFADYEMSWVRQAPGQGLEWVSL |
| | ISGDGGSTYYADSVKGRFTISRDNS |
| | KNTLYLQMNTLRAEDTAVYYCARDP |
| | DSGSYWGGDYWGQGTLVTVSS |
| | (SEQ ID NO: 244) |

TABLE 28
Anti-CLL-1 Binder Clone 28

| Anti-CLL-1 Binder | |
|---|---|
| Clone 28 | Sequence |
| V_{H}CDR1 | DFYFADY |
| | (SEQ ID NO: 245) |
| V_{H}CDR2 | SGDGGS |
| | (SEQ ID NO: 246) |
| V_{H}CDR3 | ARDPWDV |
| | (SEQ ID NO: 247) |
| V_{H} Nucleotide | CAGGTGCAGCTGGTGCAGTCTGGGG |
| Sequence | GAGGCTTGGTACAGCCTGGAGGGTC |
| | CCTGAGACTCTCCTGTGCAGCCTCT |
| | GATTTCTATTTCGCTGATTATGAAA |
| | TGAGCTGGGTCCGCCAGGCTCCAGG |
| | GAAGGGTCTGGAGTGGGTCTCTCTT |
| | ATTAGTGGGGATGGTGGTAGCACAT |
| | ACTATGCAGACTCGGTGAAGGGCCG |
| | ATTCACCATCTCCAGAGACAATTCC |
| | AAGAACACGCTGTATCTGCAAATGA |
| | ACACCCTGAGAGCCGAGGACACAGC |
| | CACGTATTACTGTGCAAGAGATCCA |
| | TGGGACGTCTGGGGCCAAGGGACCA |
| | CGGTCACCGTCTCCTCA |
| | (SEQ ID NO: 248) |
| V_{H} Amino Acid | QVQLVQSGGGLVQPGGSLRLSCAAS |
| Sequence | DFYFADYEMSWVRQAPGKGLEWVSL |
| | ISGDGGSTYYADSVKGRFTISRDNS |
| | KNTLYLQMNTLRAEDTATYYCARDP |
| | WDVWGQGTTVTVSS |
| | (SEQ ID NO: 249) |

TABLE 29
Anti-CLL-1 Binder Clone 29

| Anti-CLL-1 Binder | |
|---|---|
| Clone 29 | Sequence |
| V_{H}CDR1 | AFDFDSY |
| | (SEQ ID NO: 250) |
| V_{H}CDR2 | SGHGGN |
| | (SEQ ID NO: 251) |
| V_{H}CDR3 | VRGDEAEMSTIR |
| | (SEQ ID NO: 252) |
| V_{H} Nucleotide | CAGGTGCAGCTGGTGCAGTCTGGGG |
| Sequence | GAGGCTTGGTACAGCCTGGAGGGTC |
| | CCTGAGACTCTCCTGTGCAGCCTCT |
| | GCTTTCGATTTCGATTCTTATGAAA |
| | TGAGCTGGGTCCGCCAGGCTCCAGG |
| | ACAACGGCTTGAGTGGGTCTCAACT |
| | ATTAGTGGTCATGGTGGTAACACAT |
| | ACTACGCAGACTCCGTGAAGGGCCG |
| | ATTCACCATCTCCAGAGACAATTCC |
| | AAGAACACGCTGTATCTGCAAATGA |
| | ACACCCTGAGAGCCGAGGACACGGC |
| | TGTATATTACTGTGTGAGAGGGGAC |
| | GAGGCTGAGATGTCTACAATACGCT |
| | GGGGCCAGGGAACCCTGGTCACCGT |
| | CTCCTCA |
| | (SEQ ID NO: 253) |
| V_{H} Amino Acid | QVQLVQSGGGLVQPGGSLRLSCAAS |
| Sequence | AFDFDSYEMSWVRQAPGQRLEWVST |
| | ISGHGGNTYYADSVKGRFTISRDNS |
| | KNTLYLQMNTLRAEDTAVYYCVRGD |
| | EAEMSTIRWGQGTLVTVSS |
| | (SEQ ID NO: 254) |

TABLE 30
Anti-CLL-1 Binder Clone 30

| Anti-CLL-1 Binder | |
|---|---|
| Clone 30 | Sequence |
| V_{H}CDR1 | DFDFDAY |
| | (SEQ ID NO: 255) |
| V_{H}CDR2 | YSDGS |
| | (SEQ ID NO: 256) |
| V_{H}CDR3 | AREKDNGDYGDFES |
| | (SEQ ID NO: 257) |
| V_{H} Nucleotide | CAGGTGCAGCTGGTGCAGTCTGGGG |
| Sequence | GAGGCTTGGTACAGCCTGGAGGGTC |
| | CCTGAGACTCTCCTGTGCAGCCTCT |
| | GATTTCGATTTCGATGCTTATGAAA |
| | TGAGCTGGGTCCGCCAGGCTCCAGG |
| | ACAAGGGCTTGAGTGGGTCTCAGTG |
| | ATTTATAGCGATGGTAGCACATACT |
| | ACGCAGACTCCGTGAAGGGCCGATT |
| | TACCATCTCCAGAGACAATTCCAAG |
| | AACACGCTGTATCTGCAAATGAACA |
| | CCCTGAGAGCCGAGGACACAGCCAT |
| | ATATTACTGTGCGAGAGAAAAGGAC |
| | AACGGTGATTACGGGGACTTTGAGT |
| | CCTGGGGCCAGGGAACCCTGGTCAC |
| | CGTCTCCTCA |
| | (SEQ ID NO: 258) |
| V_{H} Amino Acid | QVQLVQSGGGLVQPGGSLRLSCAAS |
| Sequence | DFDFDAYEMSWVRQAPGQGLEWVSV |
| | IYSDGSTYYADSVKGRFTISRDNSK |
| | NTLYLQMNTLRAEDTAIYYCAREKD |
| | NGDYGDFESWGQGTLVTVSS |
| | (SEQ ID NO: 259) |

TABLE 31
Anti-CLL-1 Binder Clone 31

| Anti-CLL-1 Binder | |
|---|---|
| Clone 31 | Sequence |
| V_{H} CDR1 | SFAFSDY (SEQ ID NO: 260) |
| V_{H} CDR2 | SGSGGS (SEQ ID NO: 261) |
| V_{H} CDR3 | ARDPADYDILTGGDY (SEQ ID NO: 262) |
| V_{H} Nucleotide | CAGGTGCAGCTGGTGCAGTCTGGGGGAGGCTTGGTACAGCCTGGAGGGTCCC |
| Sequence | TGAGACTCTCCTGTGCAGCCTCTTCTTTCGCTTTCTCTGATTATGAAATGAGC |
| | TGGGTCCGCCAGGCTCCAGGACAAGGGCTTGAGTGGGTCTCAACTATAAGTG |
| | GTAGTGGTGGTAGCACATACTACGCAGACTCAGTGAAGGGCCGATTCACCAT |
| | CTCCAGAGACAATTCCAAGAACACGCTGTATCTGCAAATGAACACCCTGAGA |
| | GCCGAGGACACGGCTGTATATTACTGTGCGAGAGATCCAGCCGATTACGATA |
| | TTTTGACTGGTGGGGACTACTGGGGCCAGGGAACTCTGGTCACCGTCTCCTC |
| | A (SEQ ID NO: 263) |
| V_{H} Amino Acid | QVQLVQSGGGLVQPGGSLRLSCAASSFAFSDYEMSWVRQAPGQGLEWVSTISG |
| Sequence | SGGSTYYADSVKGRFTISRDNSKNTLYLQMNTLRAEDTAVYYCARDPADYDILT |
| | GGDYWGQGTLVTVSS (SEQ ID NO: 264) |

TABLE 32
Anti-CLL-1 Binder Clone 32

| Anti-CLL-1 Binder | |
|---|---|
| Clone 32 | Sequence |
| V_{H} CDR1 | DFDFSDY (SEQ ID NO: 265) |
| V_{H} CDR2 | SDSGDT (SEQ ID NO: 266) |
| V_{H} CDR3 | AKAPYYDILTGDDAFDT (SEQ ID NO: 267) |
| V_{H} Nucleotide | CAGGTGCAGCTGGTGCAGTCTGGGGGAGGCTTGGTACAGCCTGGAGGGTCCC |
| Sequence | TGAGACTCTCCTGTGCAGCCTCTGATTTCGATTTCTCTGATTATGAAATGAGC |
| | TGGGTCCGCCAGGCTCCAGGGAAGGGGCTGGAGTGGGTCTCAGCTATCAGTG |
| | ATAGTGGTGATACCACATACTACGCAGACTCGGTGAAGGGCCGATTCACCAT |
| | CTCCAGAGACAATTCCAAGAACACGCTGTATCTGCAAATGAACACCCTGAGA |
| | GCCGAGGACACAGCCTTGTATTACTGTGCGAAAGCCCCGTATTACGATATTT |
| | TGACTGGTGATGATGCTTTTGATACCTGGGGCCAAGGGACAATGGTCACCGT |
| | CTCCTCA (SEQ ID NO: 268) |
| V_{H} Amino Acid | QVQLVQSGGGLVQPGGSLRLSCAASDFDFSDYEMSWVRQAPGKGLEWVSAISD |
| Sequence | SGDTTYYADSVKGRFTISRDNSKNTLYLQMNTLRAEDTALYYCAKAPYYDILTG |
| | DDAFDTWGQGTMVTVSS (SEQ ID NO: 269) |

TABLE 33
Anti-CLL-1 Binder Clone 33

| Anti-CLL-1 Binder | |
|---|---|
| Clone 33 | Sequence |
| V_{H} CDR1 | YFDFDAY (SEQ ID NO: 271) |
| V_{H} CDR2 | SGDGGS (SEQ ID NO: 272) |
| V_{H} CDR3 | VRDSGYSSFH (SEQ ID NO: 273) |
| V_{H} Nucleotide | CAGGTGCAGCTGGTGCAGTCTGGGGGAGGCTTGGTACAGCCTGGAGGGTCCC |
| Sequence | TGAGACTCTCCTGTGCAGCCTCTTATTTCGATTTCGATGCTTATGAAATGAGC |
| | TGGGTCCGCCAGGCTCCAGGGAAGGGTCTGGAGTGGGTCTCTCTTATTAGTG |
| | GGGATGGTGGTAGCACATACTATGCAGACTCTGTGAAGGGCCGATTCACCAT |
| | CTCCAGAGACAATTCCAAGAACACGCTGTATCTGCAAATGAATACCCTGAGA |
| | GCCGAGGACACGGCCGTTTATTTTTGTGTGAGAGATAGCGGCTACAGCTCTT |
| | TCCACTGGGGCCAGGGAACCCAGGTCACCGTCTCCTCAAGT (SEQ ID NO: |
| | 274) |
| V_{H} Amino Acid | QVQLVQSGGGLVQPGGSLRLSCAASYFDFDAYEMSWVRQAPGKGLEWVSLISG |
| Sequence | DGGSTYYADSVKGRFTISRDNSKNTLYLQMNTLRAEDTAVYFCVRDSGYSSFH |
| | WGQGTQVTVSSS (SEQ ID NO: 275) |

TABLE 34
Anti-CLL-1 Binder Clone 34

| Anti-CLL-1 Binder | |
|---|---|
| Clone 34 | Sequence |
| V_{H} CDR1 | YFDFAYY (SEQ ID NO: 276) |
| V_{H} CDR2 | SGDGGS (SEQ ID NO: 277) |
| V_{H} CDR3 | ARDPYYGGEYYFDY (SEQ ID NO: 278) |
| V_{H} Nucleotide | CAGGTGCAGCTGGTGCAGTCTGGGGGAGGCTTGGTACAGCCTGGAGGGTCCC |
| Sequence | TGAGACTCTCCTGTGCAGCCTCTTATTTCGATTTCGCTTATTATGAAATGAGC |
| | TGGGTCCGCCAGGCTCCAGGGAAGGGTCTGGAGTGGGTCTCTCTTATTAGTG |
| | GGGATGGTGGTAGCACATACTATGCAGACTCTGTGAAGGGCCGATTCACCAT |
| | CTCCAGAGACAATTCCAAGAACACGCTGTATCTGCAAATGAACACCCTGAGA |
| | GCCGAGGACACGGCCGTATATTACTGTGCAAGAGACCCCTACTACGGTGGCG |
| | AATATTACTTTGACTACTGGGGCCAGGGAACCCTGGTCACCGTCTCCTCA |
| | (SEQ ID NO: 279) |
| V_{H} Amino Acid | QVQLVQSGGGLVQPGGSLRLSCAASYFDFAYYEMSWVRQAPGKGLEWVSLISG |
| Sequence | DGGSTYYADSVKGRFTISRDNSKNTLYLQMNTLRAEDTAVYYCARDPYYGGEY |
| | YFDYWGQGTLVTVSS (SEQ ID NO: 280) |

TABLE 35
Anti-CLL-1 Binder Clone 35

| Anti-CLL-1 Binder | |
|---|---|
| Clone 35 | Sequence |
| V_{H} CDR1 | AFAFSSY (SEQ ID NO: 281) |
| V_{H} CDR2 | SGDGGS (SEQ ID NO: 282) |
| V_{H} CDR3 | VRETESQDVVY (SEQ ID NO: 283) |
| V_{H} Nucleotide | CAGGTGCAGCTGGTGCAGTCTGGGGGAGGCTTGGTACAGCCTGGAGGGTCCC |
| Sequence | TGAGACTCTCCTGTGCAGCCTCTGCTTTCGCTTTCTCTTCTTATGAAATGAGC |
| | TGGGTCCGCCAGGCTCCAGGGAAGGGGCTGGAGTGGGTCTCTCTTATTAGTG |
| | GGGATGGTGGTAGCACATACTATGCAGACTCTGTGAAGGGCCGATTCACCAT |
| | CTCCAGAGACAATACCAAGAACACGCTGTATCTGCAAATGAACACCCTGAG |
| | AGCCGAGGACACAGCTGTATATTACTGTGTGAGAGAGACTGAATCACAGGA |
| | CGTCGTCTACTGGGGCCAGGGAACCCTGGTCACCGTCTCCTCA (SEQ ID NO: |
| | 284) |
| V_{H} Amino Acid | QVQLVQSGGGLVQPGGSLRLSCAASAFAFSSYEMSWVRQAPGKGLEWVSLISG |
| Sequence | DGGSTYYADSVKGRFTISRDNTKNTLYLQMNTLRAEDTAVYYCVRETESQDVV |
| | YWGQGTLVTVSS (SEQ ID NO: 285) |

TABLE 36
Anti-CLL-1 Binder Clone 36

| Anti-CLL-1 Binder | |
|---|---|
| Clone 36 | Sequence |
| V_{H} CDR1 | DFDFSDY (SEQ ID NO: 286) |
| V_{H} CDR2 | YSDGG (SEQ ID NO: 287) |
| V_{H} CDR3 | ARDPYYYGSGNLRFNDY (SEQ ID NO: 288) |
| V_{H} Nucleotide | CAGGTGCAGCTGGTGCAGTCTGGGGGAGGCTTGGTACAGCCTGGAGGGTCCC |
| Sequence | TGAGACTCTCCTGTGCAGCCTCTGATTTCGATTTCTCTGATTATGAAATGAGC |
| | TGGGTCCGCCAGGCTCCAGGGAAGGGGCTAGAGTGGGTCTCAGGGATTTATA |
| | GCGATGGTGGCACATACTACTCAGACTCGGTGAAGGGCCGATTCTCCATCTC |
| | CAGAGACAATTCCAAGAACACCCTGTATCTGCAAATGAACACCCTGAGAGCC |
| | GAGGACACGGCCGTATATTACTGTGCGAGAGATCCGTATTACTATGGTTCGG |
| | GGAACCTAAGATTCAATGACTACTGGGGCCAGGGAACCCTGGTCACCGTCTC |
| | CTCA (SEQ ID NO: 289) |
| V_{H} Amino Acid | QVQLVQSGGGLVQPGGSLRLSCAASDFDFSDYEMSWVRQAPGKGLEWVSGIYS |
| Sequence | DGGTYYSDSVKGRFSISRDNSKNTLYLQMNTLRAEDTAVYYCARDPYYYGSGN |
| | LRFNDYWGQGTLVTVSS (SEQ ID NO: 290) |

TABLE 37
Anti-CLL-1 Binder Clone 37

| Anti-CLL-1 Binder | |
|---|---|
| Clone 37 | Sequence |
| V_{H} CDR1 | SFDFDDY (SEQ ID NO: 291) |
| V_{H} CDR2 | SGDGGS (SEQ ID NO: 292) |
| V_{H} CDR3 | ARDPFANQQGGDY (SEQ ID NO: 293) |
| V_{H} Nucleotide | CAGGTGCAGCTGGTGCAGTCTGGGGGAGGCTTGGTACAGCCTGGAGGGTCCC |
| Sequence | TGAGACTCTCCTGTGCAGCCTCTTCTTTCGATTTCGATGATTATGAAATGAGC |
| | TGGGTCCGCCAGGCTCCAGGGAAGGGACTAGAGTGGGTCTCTCTTATTAGTG |
| | GGGATGGTGGTAGCACATACTATGCAGACTCCGTGAAGGGCCGATTCACCAT |
| | CTCCAGAGACAATTCCAAGAACACGCTGTATCTGCAAATGAACACCCTGAGA |
| | GCCGAGGACACAGCCGTGTATTACTGTGCGAGAGATCCATTTGCCAACCAGC |
| | AGGGCGGGGACTACTGGGGCCAGGGAACCCTGGTCACCGTCTCCTCA (SEQ |
| | ID NO: 294) |
| V_{H} Amino Acid | QVQLVQSGGGLVQPGGSLRLSCAASSFDFDDYEMSWVRQAPGKGLEWVSLISG |
| Sequence | DGGSTYYADSVKGRFTISRDNSKNTLYLQMNTLRAEDTAVYYCARDPFANQQG |
| | GDYWGQGTLVTVSS (SEQ ID NO: 295) |

TABLE 38
Anti-CLL-1 Binder Clone 38

| Anti-CLL-1 Binder | |
|---|---|
| Clone 38 | Sequence |
| V_{L} CDR1 | RASQSISSWLA (SEQ ID NO: 296) |
| V_{L} CDR2 | TASSLQS (SEQ ID NO: 297) |
| V_{L} CDR3 | QQSYSTPLT (SEQ ID NO: 298) |
| V_{H} CDR1 | GYTFTGY (SEQ ID NO: 299) |
| V_{H} CDR2 | NPNSGG (SEQ ID NO: 300) |
| V_{H} CDR3 | ARGDTGDFFDH (SEQ ID NO: 301) |
| V_{L} Nucleotide | GACATCCAGATGACCCAGTCCCCTTCCACCCTGTCTGCATCTGTAGGAGACA |
| Sequence | GAGTCACCATCACTTGCCGGGCTAGTCAGAGTATTAGTAGCTGGTTGGCCTG |
| | GTATCAGCAGAAACCAGGGAAAGCCCCTAAGCTCCTGATCTATACTGCATCC |
| | AGTTTGCAAAGTGGGGTCCCATCAAGGTTCAGTGGCAGTGAATCTGGGACAG |
| | ATTTCACTCTCACCATCAGCAGTCTGCAACCTGAAGATTTTGCAACTTACTAC |
| | TGTCAACAGAGTTACAGTACCCCTCTCACTTTCGGCGGAGGGACCAAAGTGG |
| | ATATCAAA (SEQ ID NO: 302) |
| V_{L} Amino Acid | DIQMTQSPSTLSASVGDRVTITCRASQSISSWLAWYQQKPGKAPKLLIYTASSLQ |
| Sequence | SGVPSRFSGSESGTDFTLTISSLQPEDFATYYCQQSYSTPLTFGGGTKVDIK (SEQ |
| | ID NO: 303) |
| Linker Nucleotide | GGTGGAGGCGGTTCCGGAGGCGGTGGCAGCGGCGGTGGCGGTTCG (SEQ ID |
| Sequence | NO: 304) |
| Linker Amino Acid | GGGGSGGGGSGGGGS (SEQ ID NO: 305) |
| Sequence | |
| V_{H} Nucleotide | CAGGTGCAGCTGGTGCAGTCTGGGGCTGAGGTGAAGAAGCCTGGGGCCTCA |
| Sequence | GTGAAGGTCTCCTGCAAGGCTTCTGGATACACCTTCACCGGCTACTATATGC |
| | ACTGGGTGCGACAGGCCCCTGGACAAGGGCTTGAGTGGATGGGATGGATCA |
| | ACCCTAACAGTGGTGGCACAAACTATGCACAGAAGTTCCAGGGCAGAGTCA |
| | CCCTGACCAGGGATACCTCCATCACCACAGCCTACTTGGAGCTGACCAGCCT |
| | GACATCTGACGACACGGCCGTCTATTACTGTGCCAGAGGTGACACAGGCGAC |
| | TTCTTTGACCATTGGGGCCAGGGAACCCTGGTCACCGTCTCCTCA (SEQ ID |
| | NO: 306) |
| V_{H} Amino Acid | QVQLVQSGAEVKKPGASVKVSCKASGYTFTGYYMHWVRQAPGQGLEWMGWI |
| Sequence | NPNSGGTNYAQKFQGRVTLTRDTSITTAYLELTSLTSDDTAVYYCARGDTGDFF |
| | DHWGQGTLVTVSS (SEQ ID NO: 307) |
| scFv Nucleotide | GACATCCAGATGACCCAGTCCCCTTCCACCCTGTCTGCATCTGTAGGAGACA |
| Sequence | GAGTCACCATCACTTGCCGGGCTAGTCAGAGTATTAGTAGCTGGTTGGCCTG |
| | GTATCAGCAGAAACCAGGGAAAGCCCCTAAGCTCCTGATCTATACTGCATCC |
| | AGTTTGCAAAGTGGGGTCCCATCAAGGTTCAGTGGCAGTGAATCTGGGACAG |
| | ATTTCACTCTCACCATCAGCAGTCTGCAACCTGAAGATTTTGCAACTTACTAC |
| | TGTCAACAGAGTTACAGTACCCCTCTCACTTTCGGCGGAGGGACCAAAGTGG |
| | ATATCAAAGGTGGAGGCGGTTCCGGAGGCGGTGGCAGCGGCGGTGGCGGTT |
| | CGCAGGTGCAGCTGGTGCAGTCTGGGGCTGAGGTGAAGAAGCCTGGGGCCT |
| | CAGTGAAGGTCTCCTGCAAGGCTTCTGGATACACCTTCACCGGCTACTATAT |
| | GCACTGGGTGCGACAGGCCCCTGGACAAGGGCTTGAGTGGATGGGATGGAT |
| | CAACCCTAACAGTGGTGGCACAAACTATGCACAGAAGTTCCAGGGCAGAGT |
| | CACCCTGACCAGGGATACCTCCATCACCACAGCCTACTTGGAGCTGACCAGC |
| | CTGACATCTGACGACACGGCCGTCTATTACTGTGCCAGAGGTGACACAGGCG |
| | ACTTCTTTGACCATTGGGGCCAGGGAACCCTGGTCACCGTCTCCTCA (SEQ ID |
| | NO: 308) |
| scFv Amino Acid | DIQMTQSPSTLSASVGDRVTITCRASQSISSWLAWYQQKPGKAPKLLIYTASSLQ |
| Sequence | SGVPSRFSGSESGTDFTLTISSLQPEDFATYYCQQSYSTPLTFGGGTKVDIKGGGG |
| | SGGGGSGGGGSQVQLVQSGAEVKKPGASVKVSCKASGYTFTGYYMHWVRQAP |
| | GQGLEWMGWINPNSGGTNYAQKFQGRVTLTRDTSITTAYLELTSLTSDDTAVY |
| | YCARGDTGDFFDHWGQGTLVTVSS (SEQ ID NO: 309) |

TABLE 39
Anti-CLL-1 Binder Clone 39

| Anti-CLL-1 Binder | |
|---|---|
| Clone 39 | Sequence |
| V_{L} CDR1 | RASQSISSYLN (SEQ ID NO: 310) |
| V_{L} CDR2 | AASSLQS (SEQ ID NO: 311) |
| V_{L} CDR3 | QQSYSTPIT (SEQ ID NO: 312) |
| V_{H} CDR1 | GYTFTGY (SEQ ID NO: 313) |
| V_{H} CDR2 | NPSSGN (SEQ ID NO: 314) |
| V_{H} CDR3 | ARGDTGDSFDY (SEQ ID NO: 315) |
| V_{L} Nucleotide | GACATCCAGATGACCCAGTCTCCATCCTCCCTGTCTGCATCTGTAGGAGACA |
| Sequence | GAGTCACCACCACTTGCCGGGCAAGTCAGAGCATTAGCAGCTATTTAAATTG |
| | GTATCAGCAGAAACCAGGGAAAGCCCCTAAGCTCCTGATCTATGCTGCATCC |
| | AGTTTGCAAAGTGGGGTCCCATCAAGGTTCAGTGGCAGTGGATCTGGGACAG |
| | ATTTCACTCTCACCATCAGCAGTCTGCAACCTGAAGATTTTGCAACTTACTAC |
| | TGTCAACAGAGTTACAGTACCCCGATCACCTTCGGCCAAGGGACACGACTGG |
| | AGATTAAA (SEQ ID NO: 316) |
| V_{L} Amino Acid | DIQMTQSPSSLSASVGDRVTTTCRASQSISSYLNWYQQKPGKAPKLLIYAASSLQ |
| Sequence | SGVPSRFSGSGSGTDFTLTISSLQPEDFATYYCQQSYSTPITFGQGTRLEIK (SEQ |
| | ID NO: 317) |
| Linker Nucleotide | GGTGGAGGCGGTTCCGGAGGCGGTGGCAGCGGCGGTGGCGGTTCG (SEQ ID |
| Sequence | NO: 318) |
| Linker Amino Acid | GGGGSGGGGSGGGGS (SEQ ID NO: 319) |
| Sequence | |
| V_{H} Nucleotide | CAGGTGCAGCTGGTGCAGTCTGGGGCTGAGGTGAAGAAGCCTGGGGCCTCA |
| Sequence | GTGAAGGTCTCCTGCAAGGCTTCTGGATACACCTTCACCGGCTACTATATGC |
| | ACTGGGTGCGACAGGCCCCTGGACAAGGGCTTGAGTGGATGGGATGGATGA |
| | ACCCGAGCAGTGGAAATACCGGCTATGCACATGAATTCCAGGGCAGAGTCA |
| | CCATGACCAGGAACACTTCCACAGGCACGGCCTACATGGAGCTGAGCGGCCT |
| | GAGATCTGAGGACACGGCCGTGTATTACTGTGCTAGAGGTGATACTGGGGAC |
| | TCCTTTGACTACTGGGGCCAGGGAACCCTGGTCACCGTCTCCTCA (SEQ ID |
| | NO: 320) |
| V_{H} Amino Acid | QVQLVQSGAEVKKPGASVKVSCKASGYTFTGYYMHWVRQAPGQGLEWMGW |
| Sequence | MNPSSGNTGYAHEFQGRVTMTRNTSTGTAYMELSGLRSEDTAVYYCARGDTG |
| | DSFDYWGQGTLVTVSS (SEQ ID NO: 321) |
| scFv Nucleotide | GACATCCAGATGACCCAGTCTCCATCCTCCCTGTCTGCATCTGTAGGAGACA |
| Sequence | GAGTCACCACCACTTGCCGGGCAAGTCAGAGCATTAGCAGCTATTTAAATTG |
| | GTATCAGCAGAAACCAGGGAAAGCCCCTAAGCTCCTGATCTATGCTGCATCC |
| | AGTTTGCAAAGTGGGGTCCCATCAAGGTTCAGTGGCAGTGGATCTGGGACAG |
| | ATTTCACTCTCACCATCAGCAGTCTGCAACCTGAAGATTTTGCAACTTACTAC |
| | TGTCAACAGAGTTACAGTACCCCGATCACCTTCGGCCAAGGGACACGACTGG |
| | AGATTAAAGGTGGAGGCGGTTCCGGAGGCGGTGGCAGCGGCGGTGGCGGTT |
| | CGCAGGTGCAGCTGGTGCAGTCTGGGGCTGAGGTGAAGAAGCCTGGGGCCT |
| | CAGTGAAGGTCTCCTGCAAGGCTTCTGGATACACCTTCACCGGCTACTATAT |
| | GCACTGGGTGCGACAGGCCCCTGGACAAGGGCTTGAGTGGATGGGATGGAT |
| | GAACCCGAGCAGTGGAAATACCGGCTATGCACATGAATTCCAGGGCAGAGT |
| | CACCATGACCAGGAACACTTCCACAGGCACGGCCTACATGGAGCTGAGCGG |
| | CCTGAGATCTGAGGACACGGCCGTGTATTACTGTGCTAGAGGTGATACTGGG |
| | GACTCCTTTGACTACTGGGGCCAGGGAACCCTGGTCACCGTCTCCTCA (SEQ |
| | ID NO: 322) |
| scFv Amino Acid | DIQMTQSPSSLSASVGDRVTTTCRASQSISSYLNWYQQKPGKAPKLLIYAASSLQ |
| Sequence | SGVPSRFSGSGSGTDFTLTISSLQPEDFATYYCQQSYSTPITFGQGTRLEIKGGGGS |
| | GGGGSGGGGSQVQLVQSGAEVKKPGASVKVSCKASGYTFTGYYMHWVRQAP |
| | GQGLEWMGWMNPSSGNTGYAHEFQGRVTMTRNTSTGTAYMELSGLRSEDTAV |
| | YYCARGDTGDSFDYWGQGTLVTVSS (SEQ ID NO: 323) |

TABLE 40
Anti-CLL-1 Binder Clone 40

| Anti-CLL-1 Binder | |
|---|---|
| Clone 40 | Sequence |
| V_{L} CDR1 | SGSSSNIGNNYVS (SEQ ID NO: 324) |
| V_{L} CDR2 | DNNKRPS (SEQ ID NO: 325) |
| V_{L} CDR3 | GTWDSSLSAWV (SEQ ID NO: 326) |
| V_{H} CDR1 | GDSVSSN (SEQ ID NO: 327) |
| V_{H} CDR2 | HYRSKWY (SEQ ID NO: 328) |
| V_{H} CDR3 | ARDRYTVRPDAFDI (SEQ ID NO: 329) |
| V_{L} Nucleotide | CAGTCTGTGTTGACGCAGCCGCCCTCAGTGTCTGCGGCCCCAGGACAGAAGG |
| Sequence | TCACCATCTCCTGCTCTGGAAGCAGCTCCAACATTGGGAATAATTATGTATC |
| | CTGGTACCAGCAGCTCCCAGGAACAGCCCCCAAACTCCTCATTTATGACAAT |
| | AATAAGCGACCCTCAGGGATTCCTGACCGATTCTCTGGCTCCGAGTCTGGCA |
| | CGTCAGCCACCCTGGGCATCACCGGACTCCAGACTGGGGACGAGGCCGATT |
| | ATTACTGCGGAACATGGGATAGCAGCCTGAGTGCTTGGGTGTTCGGCGGAGG |
| | AACCCAGCTGATCATTTTA (SEQ ID NO: 330) |
| V_{L} Amino Acid | QSVLTQPPSVSAAPGQKVTISCSGSSSNIGNNYVSWYQQLPGTAPKLLIYDNNKR |
| Sequence | PSGIPDRFSGSESGTSATLGITGLQTGDEADYYCGTWDSSLSAWVFGGGTQLIIL |
| | (SEQ ID NO: 331) |
| Linker Nucleotide | GGTGGAGGCGGTTCCGGAGGCGGTGGCAGCGGCGGTGGCGGTTCG (SEQ ID |
| Sequence | NO: 332) |
| Linker Amino Acid | GGGGSGGGGSGGGGS (SEQ ID NO: 333) |
| Sequence | |
| V_{H} Nucleotide | CAGGTACAGCTGCAGCAGTCAGGTCCAGGACTGGTGAAGCCCTCGCAGACC |
| Sequence | CTCTCACTCACCTGTGCCATCTCCGGAGACAGTGTCTCTAGCAACAGTGCTGC |
| | TTGGAACTGGATCAGGCAGTCCCCATCGAGAGGCCTTGAGTGGCTGGGACGG |
| | ACACACTACAGGTCCAAGTGGTATAATGATTATGCAGTATCTGTGAAAAGTC |
| | GAATAACCATCAGCGCAGACACTTCCAAGAACCAATTCTCCCTGCAGCTGAA |
| | CTCTGTGACTCCCGAGGACACGGCTGTGTATTACTGTGCAAGAGATCGTTAT |
| | ACGGTCCGCCCTGATGCTTTTGATATCTGGGGCCAAGGGACAACGGTCACCG |
| | TCTCCTCA (SEQ ID NO: 334) |
| V_{H} Amino Acid | QVQLQQSGPGLVKPSQTLSLTCAISGDSVSSNSAAWNWIRQSPSRGLEWLGRTH |
| Sequence | YRSKWYNDYAVSVKSRITISADTSKNQFSLQLNSVTPEDTAVYYCARDRYTVRP |
| | DAFDIWGQGTTVTVSS (SEQ ID NO: 335) |
| scFv Nucleotide | CAGTCTGTGTTGACGCAGCCGCCCTCAGTGTCTGCGGCCCCAGGACAGAAGG |
| Sequence | TCACCATCTCCTGCTCTGGAAGCAGCTCCAACATTGGGAATAATTATGTATCC |
| | TGGTACCAGCAGCTCCCAGGAACAGCCCCCAAACTCCTCATTTATGACAATA |
| | ATAAGCGACCCTCAGGGATTCCTGACCGATTCTCTGGCTCCGAGTCTGGCAC |
| | GTCAGCCACCCTGGGCATCACCGGACTCCAGACTGGGGACGAGGCCGATTAT |
| | TACTGCGGAACATGGGATAGCAGCCTGAGTGCTTGGGTGTTCGGCGGAGGA |
| | ACCCAGCTGATCATTTTAGGTGGAGGCGGTTCCGGAGGCGGTGGCAGCGGCG |
| | GTGGCGGTTCGCAGGTACAGCTGCAGCAGTCAGGTCCAGGACTGGTGAAGC |
| | CCTCGCAGACCCTCTCACTCACCTGTGCCATCTCCGGAGACAGTGTCTCTAGC |
| | AACAGTGCTGCTTGGAACTGGATCAGGCAGTCCCCATCGAGAGGCCTTGAGT |
| | GGCTGGGACGGACACACTACAGGTCCAAGTGGTATAATGATTATGCAGTATC |
| | TGTGAAAAGTCGAATAACCATCAGCGCAGACACTTCCAAGAACCAATTCTCC |
| | CTGCAGCTGAACTCTGTGACTCCCGAGGACACGGCTGTGTATTACTGTGCAA |
| | GAGATCGTTATACGGTCCGCCCTGATGCTTTTGATATCTGGGGCCAAGGGAC |
| | AACGGTCACCGTCTCCTCA (SEQ ID NO: 336) |
| scFv Amino Acid | QSVLTQPPSVSAAPGQKVTISCSGSSSNIGNNYVSWYQQLPGTAPKLLIYDNNKR |
| Sequence | PSGIPDRFSGSESGTSATLGITGLQTGDEADYYCGTWDSSLSAWVFGGGTQLIILG |
| | GGGSGGGGSGGGGSQVQLQQSGPGLVKPSQTLSLTCAISGDSVSSNSAAWNWIR |
| | QSPSRGLEWLGRTHYRSKWYNDYAVSVKSRITISADTSKNQFSLQLNSVTPEDT |
| | AVYYCARDRYTVRPDAFDIWGQGTTVTVSS (SEQ ID NO: 337) |

TABLE 41
Anti-CLL-1 Binder Clone 41

| Anti-CLL-1 Binder | |
|---|---|
| Clone 41 | Sequence |
| V_{L} CDR1 | TGTSRDVGGYNFVS (SEQ ID NO: 338) |
| V_{L} CDR2 | DVTTRPS (SEQ ID NO: 339) |
| V_{L} CDR3 | GSYTASDTML (SEQ ID NO: 340) |
| V_{H} CDR1 | GFTFDDY (SEQ ID NO: 341) |
| V_{H} CDR2 | SWNSGS (SEQ ID NO: 342) |
| V_{H} CDR3 | AREGRPTAFDY (SEQ ID NO: 343) |
| V_{L} Nucleotide | CAGTCTGTGCTGACTCAGCCTGCCTCCGTGTCTGGGTCTCTTGGACAGTCGAT |
| Sequence | CACCATCTCCTGCACTGGAACCAGCCGTGACGTTGGTGGTTATAACTTTGTCT |
| | CCTGGTACCAACAACACCCAGGCAAAGCCCCCAAACTCATAATTTTTGATGT |
| | CACTACTCGGCCCTCAGGGCTTTCTGATCGCTTCTCTGCTTCCAAGTCCGGCA |
| | ACACGGCCTCCCTGACCATCTCTGGCCTCCAGGCGGAGGACGAGGCTGATTA |
| | TTTCTGTGGCTCATACACAGCCAGTGACACTATGTTATTCGGCGGAGGGACC |
| | AAGCTGACCGTCCTA (SEQ ID NO: 344) |
| V_{L} Amino Acid | QSVLTQPASVSGSLGQSITISCTGTSRDVGGYNFVSWYQQHPGKAPKLIIFDVTTR |
| Sequence | PSGLSDRFSASKSGNTASLTISGLQAEDEADYFCGSYTASDTMLFGGGTKLTVL |
| | (SEQ ID NO: 345) |
| Linker Nucleotide | GGTGGAGGCGGTTCCGGAGGCGGTGGCAGCGGCGGTGGCGGTTCG (SEQ ID |
| Sequence | NO: 346) |
| Linker Amino Acid | GGGGSGGGGSGGGGS (SEQ ID NO: 347) |
| Sequence | |
| V_{H} Nucleotide | CAGGTGCAGCTGTTGGAGTCTGGGGGAGGCGTGGTCCAGCCTGGCAGGTCCC |
| Sequence | TGAGACTCTCCTGTGCAGCCTCTGGATTCACCTTTGATGATTATGCCATGCAC |
| | TGGGTCCGGCAAGCTCCAGGGAAGGGCCTGGAGTGGGTCTCAGGTATTAGTT |
| | GGAATAGTGGTAGCATAGGCTATGCGGACTCTGTGAAGGGCCGATTCACCGT |
| | CTCCAGAGACAATTCCAAGAACACGCTGTATCAGCAAATGAACGGCCTGAG |
| | AGCTGAAGACACGGCTGTATATTACTGTGCGAGAGAGGGGCGGCCAACGGC |
| | CTTTGACTACTGGGGCCAGGGAACCACGGTCACCGTCTCCTCA (SEQ ID NO: |
| | 348) |
| V_{H} Amino Acid | QVQLLESGGGVVQPGRSLRLSCAASGFTFDDYAMHWVRQAPGKGLEWVSGIS |
| Sequence | WNSGSIGYADSVKGRFTVSRDNSKNTLYQQMNGLRAEDTAVYYCAREGRPTAF |
| | DYWGQGTTVTVSS (SEQ ID NO: 349) |
| scFv Nucleotide | CAGTCTGTGCTGACTCAGCCTGCCTCCGTGTCTGGGTCTCTTGGACAGTCGAT |
| Sequence | CACCATCTCCTGCACTGGAACCAGCCGTGACGTTGGTGGTTATAACTTTGTCT |
| | CCTGGTACCAACAACACCCAGGCAAAGCCCCCAAACTCATAATTTTTGATGT |
| | CACTACTCGGCCCTCAGGGCTTTCTGATCGCTTCTCTGCTTCCAAGTCCGGCA |
| | ACACGGCCTCCCTGACCATCTCTGGCCTCCAGGCGGAGGACGAGGCTGATTA |
| | TTTCTGTGGCTCATACACAGCCAGTGACACTATGTTATTCGGCGGAGGGACC |
| | AAGCTGACCGTCCTAGGTGGAGGCGGTTCCGGAGGCGGTGGCAGCGGCGGT |
| | GGCGGTTCGCAGGTGCAGCTGTTGGAGTCTGGGGGAGGCGTGGTCCAGCCTG |
| | GCAGGTCCCTGAGACTCTCCTGTGCAGCCTCTGGATTCACCTTTGATGATTAT |
| | GCCATGCACTGGGTCCGGCAAGCTCCAGGGAAGGGCCTGGAGTGGGTCTCA |
| | GGTATTAGTTGGAATAGTGGTAGCATAGGCTATGCGGACTCTGTGAAGGGCC |
| | GATTCACCGTCTCCAGAGACAATTCCAAGAACACGCTGTATCAGCAAATGAA |
| | CGGCCTGAGAGCTGAAGACACGGCTGTATATTACTGTGCGAGAGAGGGGCG |
| | GCCAACGGCCTTTGACTACTGGGGCCAGGGAACCACGGTCACCGTCTCCTCA |
| | (SEQ ID NO: 350) |
| scFv Amino Acid | QSVLTQPASVSGSLGQSITISCTGTSRDVGGYNFVSWYQQHPGKAPKLIIFDVTTR |
| Sequence | PSGLSDRFSASKSGNTASLTISGLQAEDEADYFCGSYTASDTMLFGGGTKLTVLG |
| | GGGSGGGGSGGGGSQVQLLESGGGVVQPGRSLRLSCAASGFTFDDYAMHWVR |
| | QAPGKGLEWVSGISWNSGSIGYADSVKGRFTVSRDNSKNTLYQQMNGLRAEDT |
| | AVYYCAREGRPTAFDYWGQGTTVTVSS (SEQ ID NO: 351) |

TABLE 42
Anti-CLL-1 Binder Clone 42

| Anti-CLL-1 Binder | |
|---|---|
| Clone 42 | Sequence |
| V_{L} CDR1 | RASQSVSSYLA (SEQ ID NO: 352) |
| V_{L} CDR2 | DASNRAT (SEQ ID NO: 353) |
| V_{L} CDR3 | QQYGSSPVT (SEQ ID NO: 354) |
| V_{H} CDR1 | GGTFSSY (SEQ ID NO: 355) |
| V_{H} CDR2 | IPIFGT (SEQ ID NO: 356) |
| V_{H} CDR3 | ARDADEFGAFDI (SEQ ID NO: 357) |
| V_{L} Nucleotide | GAAATTGTGTTGACGCAGTCTCCAGCCACCCTGTCTTTGTCTCCAGGGGAAA |
| Sequence | GAGCCACCCTCTCCTGCAGGGCCAGTCAGAGTGTTAGCAGCTACTTAGCCTG |
| | GTACCAACAGAAACCTGGCCAGGCTCCCAGGCTCCTCATCTATGATGCATCC |
| | AACAGGGCCACTGGCATCCCAGACAGGTTCAGTGGCAGTGGGTCTGGGACA |
| | GACTTCACTCTCACCATCAGCAGACTGGAGCCTGAAGATTTTGCAGTGTATT |
| | ACTGTCAGCAGTATGGTAGCTCACCCGTCACTTTTGGCCAGGGGACCAAGCT |
| | GGAGATCAAA (SEQ ID NO: 358) |
| V_{L} Amino Acid | EIVLTQSPATLSLSPGERATLSCRASQSVSSYLAWYQQKPGQAPRLLIYDASNRA |
| Sequence | TGIPDRFSGSGSGTDFTLTISRLEPEDFAVYYCQQYGSSPVTFGQGTKLEIK (SEQ |
| | ID NO: 359) |
| Linker Nucleotide | GGTGGAGGCGGTTCCGGAGGCGGTGGCAGCGGCGGTGGCGGTTCG (SEQ ID |
| Sequence | NO: 360) |
| Linker Amino Acid | GGGGSGGGGSGGGGS (SEQ ID NO: 361) |
| Sequence | |
| V_{H} Nucleotide | CAGGTGCAGCTGGTGCAGTCTGGGGCTGAGGTGAAGAAGCCTGGGTCCTCG |
| Sequence | GTGAAGGTCTCCTGCAAGGCTTCTGGAGGCACCTTCAGCAGCTATGCTATCA |
| | GCTGGGTGCGACAGGCCCCTGGACAAGGGCTTGAGTGGATGGGAGGGATCA |
| | TCCCTATCTTTGGTACAGCAAACTACGCACAGAAGTTCCAGGGCAGAGTCAT |
| | CATGACCACAGACACATCCACGAGCACAGCCCACATGGAACTGAGGAGCCT |
| | GAGATCTGACGACACGGCCGTATATTACTGTGCGAGAGACGCTGATGAATTC |
| | GGAGCTTTTGATATCTGGGGCCAAGGGACAACGGTCACCGTCTCCTCG (SEQ |
| | ID NO: 362) |
| V_{H} Amino Acid | QVQLVQSGAEVKKPGSSVKVSCKASGGTFSSYAISWVRQAPGQGLEWMGGIIPI |
| Sequence | FGTANYAQKFQGRVIMTTDTSTSTAHMELRSLRSDDTAVYYCARDADEFGAFDI |
| | WGQGTTVTVSS (SEQ ID NO: 363) |
| scFv Nucleotide | GAAATTGTGTTGACGCAGTCTCCAGCCACCCTGTCTTTGTCTCCAGGGGAAA |
| Sequence | GAGCCACCCTCTCCTGCAGGGCCAGTCAGAGTGTTAGCAGCTACTTAGCCTG |
| | GTACCAACAGAAACCTGGCCAGGCTCCCAGGCTCCTCATCTATGATGCATCC |
| | AACAGGGCCACTGGCATCCCAGACAGGTTCAGTGGCAGTGGGTCTGGGACA |
| | GACTTCACTCTCACCATCAGCAGACTGGAGCCTGAAGATTTTGCAGTGTATT |
| | ACTGTCAGCAGTATGGTAGCTCACCCGTCACTTTTGGCCAGGGGACCAAGCT |
| | GGAGATCAAAGGTGGAGGCGGTTCCGGAGGCGGTGGCAGCGGCGGTGGCGG |
| | TTCGCAGGTGCAGCTGGTGCAGTCTGGGGCTGAGGTGAAGAAGCCTGGGTCC |
| | TCGGTGAAGGTCTCCTGCAAGGCTTCTGGAGGCACCTTCAGCAGCTATGCTA |
| | TCAGCTGGGTGCGACAGGCCCCTGGACAAGGGCTTGAGTGGATGGGAGGGA |
| | TCATCCCTATCTTTGGTACAGCAAACTACGCACAGAAGTTCCAGGGCAGAGT |
| | CATCATGACCACAGACACATCCACGAGCACAGCCCACATGGAACTGAGGAG |
| | CCTGAGATCTGACGACACGGCCGTATATTACTGTGCGAGAGACGCTGATGAA |
| | TTCGGAGCTTTTGATATCTGGGGCCAAGGGACAACGGTCACCGTCTCCTCG |
| | (SEQ ID NO: 364) |
| scFv Amino Acid | EIVLTQSPATLSLSPGERATLSCRASQSVSSYLAWYQQKPGQAPRLLIYDASNRA |
| Sequence | TGIPDRFSGSGSGTDFTLTISRLEPEDFAVYYCQQYGSSPVTFGQGTKLEIKGGGG |
| | SGGGGSGGGGSQVQLVQSGAEVKKPGSSVKVSCKASGGTFSSYAISWVRQAPG |
| | QGLEWMGGIIPIFGTANYAQKFQGRVIMTTDTSTSTAHMELRSLRSDDTAVYYC |
| | ARDADEFGAFDIWGQGTTVTVSS (SEQ ID NO: 365) |

TABLE 43
Anti-CLL-1 Binder Clone 43

| Anti-CLL-1 Naive | |
|---|---|
| Human Binder | |
| Clone AHP17190 | Sequence |
| V_{L} CDR 1 | QSVSTY (SEQ ID NO: 419) |
| V_{L} CDR 2 | DAS (SEQ ID NO: 420) |
| V_{L} CDR 3 | QHRTNWPMYT (SEQ ID NO: 421) |
| V_{H} CDR 1 | GFTFDDYA (SEQ ID NO: 423) |
| V_{H} CDR 2 | ISWNSGST (SEQ ID NO: 424) |
| V_{H} CDR 3 | VRDRAAMAVEHFQK (SEQ ID NO: 425) |
| V_{L} Amino Acid | DVVMTQSPATLSLSPGEGATLSCRASQSVSTYLAWYQHKPGQSPRLLVFDASNRA |
| Sequence | TGTPARFSGSGSGTDFTLTIDSLEPEDFAVYYCQHRTNWPMYTFGQGTKLEIK |
| | (SEQ ID NO: 418) |
| V_{H} Amino Acid | EVQLVESGGGLAQPGMSLRLSCVASGFTFDDYAMHWVRQVPGKGLEWVSGISW |
| Sequence | NSGSTGYADSVKGRFSISRDNAKNSLYLQINSLRPEDTALYYCVRDRAAMAVEHF |
| | QKWGQGTPVTVSS (SEQ ID NO: 422) |

TABLE 44
Anti-CLL-1 Binder Clone 44

| Anti-CLL-1 Naive | |
|---|---|
| Human Binder | |
| Clone AHP17191 | Sequence |
| V_{L} CDR 1 | QSVLYRSNNKNY (SEQ ID NO: 427) |
| V_{L} CDR 2 | WAS (SEQ ID NO: 428) |
| V_{L} CDR 3 | QQYSTTPWT (SEQ ID NO: 429) |
| V_{H} CDR 1 | GFTFSSYG (SEQ ID NO: 431) |
| V_{H} CDR 2 | ISYEGSTK (SEQ ID NO: 432) |
| V_{H} CDR 3 | TKESITFAGPNYFDF (SEQ ID NO: 433) |
| V_{L} Amino Acid | EIVMTQTPDSLAVSLGERATINCKSSQSVLYRSNNKNYLAWYQQKPGQPPKLLIH |
| Sequence | WASTRESGVPDRFSGSGSGTDFTLTIGSLQAEDVAIYYCQQYSTTPWTFGQGTKLE |
| | IK (SEQ ID NO: 426) |
| V_{H} Amino Acid | ETQLVESGGGVVQPGRSLSLSCAASGFTFSSYGMHWVRQAPGKGLEWVAVISYEG |
| Sequence | STKYYADSVKGRFTISRDNSENTLYLQMNSLRAEDTALYYCTKESITFAGPNYFDF |
| | WGQGTLVTVSS (SEQ ID NO: 430) |

TABLE 45
Anti-CLL-1 Binder Clone 45

| Anti-CLL-1 Naive | |
|---|---|
| Human Binder | |
| Clone AHP17192 | Sequence |
| V_{L} CDR 1 | QSLLHSDGKTY (SEQ ID NO: 435) |
| V_{L} CDR 2 | EVS (SEQ ID NO: 436) |
| V_{L} CDR 3 | MQSIQLQT (SEQ ID NO: 437) |
| V_{H} CDR 1 | GYTFMTYA (SEQ ID NO: 439) |
| V_{H} CDR 2 | INTNTGKP (SEQ ID NO: 440) |
| V_{H} CDR 3 | ARGDEYSGYVRLDGVDY (SEQ ID NO: 441) |
| V_{L} Amino Acid | DVVMTQSPLSLSVTPGQPASISCKSTQSLLHSDGKTYLYWYLQKPGQPPQLLIYEV |
| Sequence | SNRFSGVPDRFSGSGSGTDFTLKISRVEAEDVGVYYCMQSIQLQTFGQGTKVDIK |
| | (SEQ ID NO: 434) |
| V_{H} Amino Acid | QMQLVQSGSELKKPGASVKVSCKTSGYTFMTYAMNWVRQAPGQGLEWMGWIN |
| Sequence | TNTGKPTYDQGFTGRFVFSLDTSVSTAYLQINNLKAEDTAVYYCARGDEYSGYVR |
| | LDGVDYWGQGTLVTVSS (SEQ ID NO: 438) |

TABLE 46
Anti-CLL-1 Binder Clone 46

| Anti-CLL-1 Naïve | |
|---|---|
| Human Binder | |
| Clone AHP17194 | Sequence |
| V_{L} CDR 1 | QSVSNY (SEQ ID NO: 443) |
| V_{L} CDR 2 | DAS (SEQ ID NO: 444) |
| V_{L} CDR 3 | QQRSDWPLT (SEQ ID NO: 445) |
| V_{H} CDR 1 | GYSFTNYW (SEQ ID NO: 447) |
| V_{H} CDR 2 | IDPSDSYT (SEQ ID NO: 448) |
| V_{H} CDR 3 | ARLGSGWLQYSANDY (SEQ ID NO: 449) |
| V_{L} Amino Acid | EIVMTQSPATLSLSPGERATLSCRASQSVSNYLAWYQQKPGQAPRLLIYDASNRAT |
| Sequence | GIPARFSGRGSGTDFTLTISSLEPEDFAVYYCQQRSDWPLTFGGGTKVEIK (SEQ ID |
| | NO: 442) |
| V_{H} Amino Acid | QVQLVQSGAEVKKPGESLRISCKGSGYSFTNYWISWVRQMPGKGLEWMGRIDPS |
| Sequence | DSYTNYSPSFQGHVTISADKSISTAYLQWSSLKASDTAMYYCARLGSGWLQYSAN |
| | DYWGQGTLVTVSS (SEQ ID NO: 446) |

TABLE 47
Anti-CLL-1 Binder Clone 47

| Anti-CLL-1 Naïve | |
|---|---|
| Human Binder | |
| Clone AHP17195 | Sequence |
| V_{L} CDR 1 | QRITTF (SEQ ID NO: 451) |
| V_{L} CDR 2 | AAS (SEQ ID NO: 452) |
| V_{L} CDR 3 | QQSYGTPWT (SEQ ID NO: 453) |
| V_{H} CDR 1 | GVTSSSYG (SEQ ID NO: 455) |
| V_{H} CDR 2 | LLNDGTFE (SEQ ID NO: 456) |
| V_{H} CDR 3 | ATTPHYDDMTGFFSPFDY (SEQ ID NO: 457) |
| V_{L} Amino Acid | AIQLTQSPPSLSASVGDRVTISCRASQRITTFLNWYQQKPGKAPRLLIYAASSLQSG |
| Sequence | VPPRFSGSGSGTVFTLTISSLHPEDFATYYCQQSYGTPWTFGQGTKVEIK (SEQ ID |
| | NO: 450) |
| V_{H} Amino Acid | EVQLLESGGGVVQPGRSLRLSCAASGVTSSSYGIHWVRQAPGKGLEWLAVLLND |
| Sequence | GTFEDSRDSVQGRFTISRDKVKNTVSLQMNRLTTEDTAVYYCATTPHYDDMTGFF |
| | SPFDYWGQGTLVTVSS (SEQ ID NO: 454) |

TABLE 48
Anti-CLL-1 Binder Clone 48

| Anti-CLL-1 Naïve | |
|---|---|
| Human Binder | |
| Clone AHP17196 | Sequence |
| V_{L} CDR 1 | QNVYNS (SEQ ID NO: 459) |
| V_{L} CDR 2 | WAS (SEQ ID NO: 460) |
| V_{L} CDR 3 | QQYYSLPLT (SEQ ID NO: 461) |
| V_{H} CDR 1 | GGTFSTFG (SEQ ID NO: 463) |
| V_{H} CDR 2 | ISYDGSTK (SEQ ID NO: 464) |
| V_{H} CDR 3 | GKGLEATIMTHMDV (SEQ ID NO: 465) |
| V_{L} Amino Acid | DIVMTQSPDSLAVSLGERATINCKSSQNVYNSLTWYQQQPGQSPKVLLYWASTRE |
| Sequence | SGVPDRFSGSGSGTDFTLTISSLQAEDVAVYYCQQYYSLPLTFGGGTKVEIK (SEQ |
| | ID NO: 458) |
| V_{H} Amino Acid | QVQLVESGGGVVQPGTSLRLSCAASGGTFSTFGIHWVRQAPGKGLEWVAVISYDG |
| Sequence | STKYYADSVKGRFTISRDNSKNTVFVQMNSLRAEDTAVYYCGKGLEATIMTHMD |
| | VWGQGPLVTVSS (SEQ ID NO: 462) |

TABLE 49
Anti-CLL-1 Binder Clone 49

| Anti-CLL-1 Naïve | |
|---|---|
| Human Binder | |
| Clone AHP17198 | Sequence |
| V_{L} CDR 1 | QSVSSN (SEQ ID NO: 467) |
| V_{L} CDR 2 | GAS (SEQ ID NO: 468) |
| V_{L} CDR 3 | QQYNNWPRT (SEQ ID NO: 469) |
| V_{H} CDR 1 | GFSLSTSGMC (SEQ ID NO: 471) |
| V_{H} CDR 2 | IDWDDDK (SEQ ID NO: 472) |
| V_{H} CDR 3 | ARIRAYYDSSGYYYWYWFDP (SEQ ID NO: 473) |
| V_{L} Amino Acid | DVVMTQSPATLSVSPGERATLSCRASQSVSSNLAWYQQKPGQAPRLLIYGASTRA |
| Sequence | TGIPARFSGSGSGTEFTLTISSLQSEDFAVYYCQQYNNWPRTFGQGTKVEIK (SEQ |
| | ID NO: 466) |
| V_{H} Amino Acid | QITLKESGPALVKPTQTLTLTCTFSGFSLSTSGMCVSWIRQPPGKALEWLALIDWD |
| Sequence | DDKYYSTSLKTRLTISKDTSKNQVVLTMTNMDPVDTATYYCARIRAYYDSSGYY |
| | YWYWFDPWGQGTLVTVSS (SEQ ID NO: 470) |

TABLE 50
Anti-CLL-1 Binder Clone 50

| Anti-CLL-1 Naïve | |
|---|---|
| Human Binder | |
| AHP17199 | Sequence |
| V_{L} CDR 1 | QSVSNN (SEQ ID NO: 475) |
| V_{L} CDR 2 | GAS (SEQ ID NO: 476) |
| V_{L} CDR 3 | QQYDKWPFT (SEQ ID NO: 477) |
| V_{H} CDR 1 | GDSVSSNSAA (SEQ ID NO: 479) |
| V_{H} CDR 2 | TYYRSKWYN (SEQ ID NO: 480) |
| V_{H} CDR 3 | AREGPPYYDFWSGYLLADHYGMDV (SEQ ID NO: 481) |
| V_{L} Amino Acid | EIVLTQSPATLSVSPGERATLSCRASQSVSNNVAWYQQKPGQAPRLLLYGASTRAS |
| Sequence | GIPDRLTGSGSGTEFTLTFSSLQSEDFAVYYCQQYDKWPFTFGPGTKLEIK (SEQ ID |
| | NO: 474) |
| V_{H} Amino Acid | QVQLQQSGPGLVKPSQTLSLTCAISGDSVSSNSAAWNWIRQSPSRGLEWLGRTYY |
| Sequence | RSKWYNDYAVSVKSRITINPDTSKNQFSLQLNSVTPEDTAVYYCAREGPPYYDFW |
| | SGYLLADHYGMDVWGQGTTVTVSS (SEQ ID NO: 478) |

TABLE 51
Anti-CLL-1 Binder Clone 51

| Anti-CLL-1 Naïve | |
|---|---|
| Human Binder | |
| Clone AHP17201 | Sequence |
| V_{L} CDR 1 | QSVLYSSNNKNY (SEQ ID NO: 483) |
| V_{L} CDR 2 | WAS (SEQ ID NO: 484) |
| V_{L} CDR 3 | QQYYSTPRFT (SEQ ID NO: 485) |
| V_{H} CDR 1 | GFTFSTYG (SEQ ID NO: 487) |
| V_{H} CDR 2 | ISYDGSNT (SEQ ID NO: 488) |
| V_{H} CDR 3 | ARGGIRTVMYYFDY (SEQ ID NO: 489) |
| V_{L} Amino Acid | DIVMTQSPDSLAVSLGERATINCKSSQSVLYSSNNKNYLAWYQQKPGQPPKLLIY |
| Sequence | WASTRESGVPDRFSGSGSGTDFTLTISSLQAEDVAVYYCQQYYSTPRFTFGPGTKV |
| | EIK (SEQ ID NO: 482) |
| V_{H} Amino Acid | QVQLLESGGGVVQPGRSLRLSCAASGFTFSTYGMFWVRQAPGKGLEWVAAISYD |
| Sequence | GSNTYYADFVRGQFSISRDNPKNTLYLQMNSLRAEDTAVYYCARGGIRTVMYYF |
| | DYWGQGTLVTVSS (SEQ ID NO: 486) |

TABLE 52
Anti-CLL-1 Binder Clone 52

| Anti-CLL-1 Naïve | |
|---|---|
| Human Binder | |
| Clone AHP17202 | Sequence |
| V_{L} CDR 1 | QNLLYSSDNNNY (SEQ ID NO: 491) |
| V_{L} CDR 2 | WAS (SEQ ID NO: 492) |
| V_{L} CDR 3 | QQYYSTPLT (SEQ ID NO: 493) |
| V_{H} CDR 1 | GITFSSHW (SEQ ID NO: 495) |
| V_{H} CDR 2 | INGDGSST (SEQ ID NO: 496) |
| V_{H} CDR 3 | VRDCGDYGCY (SEQ ID NO: 497) |
| V_{L} Amino Acid | DIVMTQSPDSLTVSLGERATINCKSSQNLLYSSDNNNYLAWFQQKPGQPPKLLIHW |
| Sequence | ASTRESGVPDRFSGSGSGTEFTLTISSLQAEDVAVYYCQQYYSTPLTFGGGTKLEIK |
| | (SEQ ID NO: 490) |
| V_{H} Amino Acid | EVQLVESGGGLVQPGGSLRLSCAASGITFSSHWMHWVRQAPGKGPVWVSRINGD |
| Sequence | GSSTTYVDSVKGRFSISRDNAKNTLYLQMNSLRAEDTAVYYCVRDCGDYGCYWG |
| | QGTLVTVSS (SEQ ID NO: 494) |

TABLE 53
Anti-CLL-1 Binder Clone 53

| Anti-CLL-1 Naïve | |
|---|---|
| Human Binder | |
| Clone AHP17205 | Sequence |
| V_{L} CDR 1 | RSVNSN (SEQ ID NO: 499) |
| V_{L} CDR 2 | DAS (SEQ ID NO: 500) |
| V_{L} CDR 3 | QQYNDWPPIT (SEQ ID NO: 501) |
| V_{H} CDR 1 | GYTFTGYY (SEQ ID NO: 503) |
| V_{H} CDR 2 | INPNSGGT (SEQ ID NO: 504) |
| V_{H} CDR 3 | ARSRIYWSYDILTGYSADTWELEQKIRPGEYYYYYYGMDV (SEQ ID NO: 505) |
| V_{L} Amino Acid | DVVMTQSPATLSLSLGERATLSCRASRSVNSNLAWYQQKPGQAPRLLIYDASTRV |
| Sequence | TGISERFSGSGSGIEFTLTISSLQSEDSAVYYCQQYNDWPPITLGQGTKVEIK (SEQ |
| | ID NO: 498) |
| V_{H} Amino Acid | EVQLVQSGAEVKKPGASVKVSCKASGYTFTGYYMHWVRQAPGQGLEWMGGINP |
| Sequence | NSGGTNYAQKFQGRVTMTRDTSISTAYMELSRLRSDDTAVYYCARSRIYWSYDIL |
| | TGYSADTWELEQKIRPGEYYYYYYGMDVWGQGTTVTVSS (SEQ ID NO: 502) |

TABLE 54
Anti-CLL-1 Binder Clone 54

| Anti-CLL-1 Naïve | |
|---|---|
| Human Binder | |
| Clone AHP17207 | Sequence |
| V_{L} CDR 1 | QSVLYSSNNKNY (SEQ ID NO: 507) |
| V_{L} CDR 2 | WAS (SEQ ID NO: 508) |
| V_{L} CDR 3 | QQYYSTLPT (SEQ ID NO: 509) |
| V_{H} CDR 1 | GFNFNNAW (SEQ ID NO: 511) |
| V_{H} CDR 2 | IKTTPEGATT (SEQ ID NO: 512) |
| V_{H} CDR 3 | HTDDHNWNPHDH (SEQ ID NO: 513) |
| V_{L} Amino Acid | DIVMTQSPDSLAVSLGERATINCMSSQSVLYSSNNKNYLAWYQQKPGQPPKLLIY |
| Sequence | WASTRESGVPDRFSGSGSRTHFTLTISSLQAEDVAAYYCQQYYSTLPTFGGGTKVE |
| | IK (SEQ ID NO: 506) |
| V_{H} Amino Acid | ETQLVESGGGLVKPGGSLRVSCAVSGFNFNNAWMNWVRQTPGKGLEWVGQIKT |
| Sequence | TPEGATTDYPAPVKGRFTISRDDSKNTLSLQMASLTTEDTAIYYCHTDDHNWNPH |
| | DHWGQGTLVTVSS (SEQ ID NO: 510) |

TABLE 55
Anti-CLL-1 Binder Clone 55

| Anti-CLL-1 Naïve | |
|---|---|
| Human Binder | |
| Clone AHP17208 | Sequence |
| V_{L} CDR 1 | QSVLYSSNNKNY (SEQ ID NO: 515) |
| V_{L} CDR 2 | WAS (SEQ ID NO: 516) |
| V_{L} CDR 3 | QQYYTTPT (SEQ ID NO: 517) |
| V_{H} CDR 1 | GGTFSSYA (SEQ ID NO: 519) |
| V_{H} CDR 2 | IIPIFGTA (SEQ ID NO: 520) |
| V_{H} CDR 3 | AGGATSGSYYAGFGGCY (SEQ ID NO: 521) |
| V_{L} Amino Acid | ETTLTQSPDSLAVSLGERATINCKSSQSVLYSSNNKNYLAWYQQKPGQPPKLLIYW |
| Sequence | ASTRESGVPDRFSGSGSGTDFTLTISSLQAGDVAIYYCQQYYTTPTFGQGTRLEIK |
| | (SEQ ID NO: 514) |
| V_{H} Amino Acid | EVOLVQSGAEVKKPGSSVKVSCKASGGTFSSYAISWVRQAPGQGLEWMGGIIPIFG |
| Sequence | TANYAQKFQGRVTITADESTSTAYMELSSLRSEDTAVYYCAGGATSGSYYAGFGG |
| | CYWGQGTLVTVSS (SEQ ID NO: 518) |

TABLE 56
Anti-CLL-1 Binder Clone 56

| Anti-CLL-1 Naïve | |
|---|---|
| Human Binder | |
| Clone AHP17209 | Sequence |
| V_{L} CDR 1 | QSVGNF (SEQ ID NO: 523) |
| V_{L} CDR 2 | DAS (SEQ ID NO: 524) |
| V_{L} CDR 3 | QQRRDWPLT (SEQ ID NO: 525) |
| V_{H} CDR 1 | EYTFTNYF (SEQ ID NO: 527) |
| V_{H} CDR 2 | INPYDAIT (SEQ ID NO: 528) |
| V_{H} CDR 3 | ARASWRDVPLFDY (SEQ ID NO: 529) |
| V_{L} Amino Acid | EIVMTQSPATLSLSPGERATLFCTASQSVGNFLTWYQQKPGQPPRLLIYDASNRAA |
| Sequence | GVPARFSGSGFGTDFTLTISTLEPDDFAVYYCQQRRDWPLTFGGGTKVEIK (SEQ |
| | ID NO: 522) |
| V_{H} Amino Acid | ETQLVESGAEVKEPGASVKISCETSEYTFTNYFFHWVRQTPGHGLEWMGIINPYDA |
| Sequence | ITTSAQKFQGRVTLTWDTSTGTVYMDLNSLRFEDTATYYCARASWRDVPLFDYW |
| | GQGTLVTVSS (SEQ ID NO: 526) |

TABLE 57
Anti-CLL-1 Binder Clone 57

| Anti-CLL-1 Naïve | |
|---|---|
| Human Binder | |
| Clone AHP17210 | Sequence |
| V_{L} CDR 1 | QGISKN (SEQ ID NO: 531) |
| V_{L} CDR 2 | AAS (SEQ ID NO: 532) |
| V_{L} CDR 3 | LOHNTFPRT (SEQ ID NO: 533) |
| V_{H} CDR 1 | GYGFTSYW (SEQ ID NO: 535) |
| V_{H} CDR 2 | IYPGDSDT (SEQ ID NO: 536) |
| V_{H} CDR 3 | ARGGYYYGPGRAFDI (SEQ ID NO: 537) |
| V_{L} Amino Acid | NIQMTQSPSSLSASVGDRVTITCRASQGISKNLGWYQQKPGKAPKRLIYAASFLQS |
| Sequence | GVPSRFSGSGSGTDFTLTISSLQPEDFATYYCLQHNTFPRTFGQGTKVEIK (SEQ ID |
| | NO: 530) |
| V_{H} Amino Acid | QVQLVQSGAEVKKPGESLKISCKGSGYGFTSYWIGWVRQMPGKGLEWMGIIYPG |
| Sequence | DSDTRYSPSFQGQVTISADKSISTAYLQWSSLKASDTAMYYCARGGYYYGPGRAF |
| | DIWGQGTMVTVSS (SEQ ID NO: 534) |

TABLE 58
Anti-CLL-1 Binder Clone 58

| Anti-CLL-1 Naïve | |
|---|---|
| Human Binder | |
| Clone AHP17211 | Sequence |
| V_{L} CDR 1 | QDIDDD (SEQ ID NO: 539) |
| V_{L} CDR 2 | EAT (SEQ ID NO: 540) |
| V_{L} CDR 3 | LQHDNFPRT (SEQ ID NO: 541) |
| V_{H} CDR 1 | GYTFSNYY (SEQ ID NO: 543) |
| V_{H} CDR 2 | ITPSGDSA (SEQ ID NO: 544) |
| V_{H} CDR 3 | ASESPGSGGLEN (SEQ ID NO: 545) |
| V_{L} Amino Acid | ETTLTQSPAFMSATPGDKVNISCKASQDIDDDMNWYQQKPGEAAIFIIQEATTLVP |
| Sequence | GIPPRFSGSGYGTDFTLTINNIESEDAAYYFCLQHDNFPRTFGQGTRLEIK (SEQ ID |
| | NO: 538) |
| V_{H} Amino Acid | EVQLVQSGPEVEKPGASVMLSCKSSGYTFSNYYVHWVRQAPGQGVEWMAVITPS |
| Sequence | GDSAFFAQRFRGRLTTTRDTSTSTVYMELSSLTSEDTALYYCASESPGSGGLENWG |
| | QGTLVTVSS (SEQ ID NO: 542) |

TABLE 59
Anti-CLL-1 Binder Clone 59

| Anti-CLL-1 Naïve | |
|---|---|
| Human Binder | |
| Clone AHP17212 | Sequence |
| V_{L} CDR 1 | HSLVYSDGNTY (SEQ ID NO: 547) |
| V_{L} CDR 2 | KVS (SEQ ID NO: 548) |
| V_{L} CDR 3 | MQGTHWPPT (SEQ ID NO: 549) |
| V_{H} CDR 1 | GFTFSNYW (SEQ ID NO: 551) |
| V_{H} CDR 2 | MKQDGSDK (SEQ ID NO: 552) |
| V_{H} CDR 3 | ARAGRDYFGLGTPDF (SEQ ID NO: 553) |
| V_{L} Amino Acid | DVVMTQSPLSLPVTLGQPASISCRSSHSLVYSDGNTYLNWFQQRPGQSPRRLIYKV |
| Sequence | SNWDSGVPDRFSGSGSGTDFTLKISRVEAEDVGVYYCMQGTHWPPTFGQGTRLEI |
| | K (SEQ ID NO: 546) |
| V_{H} Amino Acid | ETQLVESGGGLVQPGGSLRLSCTASGFTFSNYWMNWVRQAPGKGLEWVANMKQ |
| Sequence | DGSDKYYANSVKGRFAISRDNARNSLFLQMNSLRAEDTAVYFCARAGRDYFGLG |
| | TPDFWGQGTLVTVSS (SEQ ID NO: 550) |

TABLE 60
Anti-CLL-1 Binder Clone 60

| Anti-CLL-1 Naïve | |
|---|---|
| Human Binder | |
| Clone AHP17213 | Sequence |
| V_{L} CDR 1 | QSVSSSY (SEQ ID NO: 555) |
| V_{L} CDR 2 | GAS (SEQ ID NO: 556) |
| V_{L} CDR 3 | QQYGSSSMYT (SEQ ID NO: 557) |
| V_{H} CDR 1 | GYQFNGYY (SEQ ID NO: 559) |
| V_{H} CDR 2 | INAYSGDT (SEQ ID NO: 560) |
| V_{H} CDR 3 | ARGRFYDFWSGYHHLDY (SEQ ID NO: 561) |
| V_{L} Amino Acid | DVVMTQSPGTLSLSPGERATLSCRASQSVSSSYLAWYQQKPGQAPRLLIYGASSRA |
| Sequence | TGIPDRFSGSGSGTDFTLTISRLEPEDFAVYYCQQYGSSSMYTFGQGTKVDIK (SEQ |
| | ID NO: 554) |
| V_{H} Amino Acid | QVQLMQSGAEVKKPGASAKVSCKASGYQFNGYYIYWVRQAPGQGLEWMGWIN |
| Sequence | AYSGDTSYPQTFQGRVTMTRDTSIDTAYMELKSLRSDDTAVYYCARGRFYDFWS |
| | GYHHLDYWGQGTLVTVSS (SEQ ID NO: 558) |

TABLE 61
Anti-CLL-1 Binder Clone 61

| Anti-CLL-1 Naïve | |
|---|---|
| Human Binder | |
| Clone AHP17214 | Sequence |
| V_{L} CDR 1 | QSVNSH (SEQ ID NO: 563) |
| V_{L} CDR 2 | EAS (SEQ ID NO: 564) |
| V_{L} CDR 3 | QQRKNWPP (SEQ ID NO: 565) |
| V_{H} CDR 1 | GFTFSSYE (SEQ ID NO: 567) |
| V_{H} CDR 2 | ISSSVSTI (SEQ ID NO: 568) |
| V_{H} CDR 3 | ARGGSRGAVAGREFDY (SEQ ID NO: 569) |
| V_{L} Amino Acid | DVVMTQSPATLSLSPGERATLSCRASQSVNSHLAWYQQKPGQAPRLLIYEASNRA |
| Sequence | TGIPARFSGSGSGTDFTLTISSLEPEDFAVYYCQQRKNWPPFGQGTRLEIK (SEQ ID |
| | NO: 562) |
| V_{H} Amino Acid | ETQLVESGGGLVQPGGSLRLSCAASGFTFSSYEMNWVRQAPGKGLEWVSYISSSV |
| Sequence | STIYYADSVKGRFTISRDNAKNSLYLQMNSLRAEDTAIYYCARGGSRGAVAGREF |
| | DYWGQGTLVTVSS (SEQ ID NO: 566) |

TABLE 62
Anti-CLL-1 Binder Clone 62

| Anti-CLL-1 Naïve | |
|---|---|
| Human Binder | |
| Clone AHP17216 | Sequence |
| V_{L} CDR 1 | SGDVGGYDF (SEQ ID NO: 571) |
| V_{L} CDR 2 | DVS (SEQ ID NO: 572) |
| V_{L} CDR 3 | TSYTTSITPLYV (SEQ ID NO: 573) |
| V_{H} CDR 1 | GYTFTEYD (SEQ ID NO: 575) |
| V_{H} CDR 2 | INAGNGDT (SEQ ID NO: 576) |
| V_{H} CDR 3 | ARRAWLQLWPHGYYFDY (SEQ ID NO: 577) |
| V_{L} Amino Acid | QSALTQPASVSGSPGQSITISCTGTSGDVGGYDFVSWYQQHPGKAPKVLIYDVSER |
| Sequence | PSGVSTRFSGSKSGNTASLTISGLQAEDEADYYCTSYTTSITPLYVFGTGTKVTVL |
| | (SEQ ID NO: 570) |
| V_{H} Amino Acid | EVQLVESGGEAKMPGGSVKISCKSSGYTFTEYDIAWVRQAPGQGLEWLGWINAG |
| Sequence | NGDTKSAHDFQGRVTLTTDTSTSTAYMELRSLKSDDTAVYFCARRAWLQLWPHG |
| | YYFDYWGQGALVTVSS (SEQ ID NO: 574) |

TABLE 63
Anti-CLL-1 Binder Clone 63

| Anti-CLL-1 Naïve | |
|---|---|
| Human Binder | |
| Clone AHP17217 | Sequence |
| V_{L} CDR 1 | RSSIGRNT (SEQ ID NO: 579) |
| V_{L} CDR 2 | KNN (SEQ ID NO: 580) |
| V_{L} CDR 3 | ATWDDSLPGPV (SEQ ID NO: 581) |
| V_{H} CDR 1 | GFTFSSYS (SEQ ID NO: 583) |
| V_{H} CDR 2 | ISATTTNI (SEQ ID NO: 584) |
| V_{H} CDR 3 | ARVDRTVRTYYYMDV (SEQ ID NO: 585) |
| V_{L} Amino Acid | QSALTQPPAASGTPGQRVTIFCSGSRSSIGRNTVNWYQQFAGEAPKLLIEKNNQRP |
| Sequence | WGVPDRFSASKSGTSASLAISGLQSEDEAHYFCATWDDSLPGPVFGGGTKVTVL |
| | (SEQ ID NO: 578) |
| V_{H} Amino Acid | EVQLVESGGGLVKPGGSLRLSCAASGFTFSSYSMNWVRLAPGKGLEWVAFISATT |
| Sequence | TNILY ADSLKGRFTISRDNAKNSLDLHINSLRADDTAVYYCARVDRTVRTYYYMD |
| | VWGPGTTVTVSS (SEQ ID NO: 582) |

TABLE 64
Anti-CLL-1 Binder Clone 64

| Anti-CLL-1 Naïve | |
|---|---|
| Human Binder | |
| Clone AHP17218 | Sequence |
| V_{L} CDR 1 | SSDVGGYNY (SEQ ID NO: 587) |
| V_{L} CDR 2 | DVS (SEQ ID NO: 588) |
| V_{L} CDR 3 | SSYRNSSTVV (SEQ ID NO: 589) |
| V_{H} CDR 1 | GFTFSTYS (SEQ ID NO: 591) |
| V_{H} CDR 2 | ISSSSSGR (SEQ ID NO: 592) |
| V_{H} CDR 3 | ARDESGLVPCDY (SEQ ID NO: 593) |
| V_{L} Amino Acid | QSALTQPASVSGSPGQSITISCAGTSSDVGGYNYVSWYQQHPGKAPRLIIYDVSNRP |
| Sequence | SGVSDRFSGSKSGNTASLTISGLQADDEADYYCSSYRNSSTVVFGPATKLTVL |
| | (SEQ ID NO: 586) |
| V_{H} Amino Acid | QVQLVESGGGLVQPGGSLRLSCAASGFTFSTYSMNWVRQAPGKGLEWVSYISSSS |
| Sequence | SGRYYADSVKGRFTISRDNAKNSLYLQMNSLRDEDTAVYYCARDESGLVPCDYW |
| | GQGTLVTVSS (SEQ ID NO: 590) |

TABLE 65
Anti-CLL-1 Binder Clone 65

| Anti-CLL-1 Naïve | |
|---|---|
| Human Binder | |
| Clone AHP17219 | Sequence |
| V_{L} CDR 1 | SSDVGGYNW (SEQ ID NO: 595) |
| V_{L} CDR 2 | DVT (SEQ ID NO: 596) |
| V_{L} CDR 3 | TSYTTRSTLV (SEQ ID NO: 597) |
| V_{H} CDR 1 | GFTFSSYA (SEQ ID NO: 599) |
| V_{H} CDR 2 | ISYDGSNK (SEQ ID NO: 600) |
| V_{H} CDR 3 | AREVVGATLDY (SEQ ID NO: 601) |
| V_{L} Amino Acid | QSALTQPASVAGSPGQSITISCTGTSSDVGGYNWVSWYQHNPHKAPKLIIFDVTQR |
| Sequence | PSGVSNRFSGSKSGNTASLTISGLQTEDEADYYCTSYTTRSTLVFGGGTKLTVL |
| | (SEQ ID NO: 594) |
| V_{H} Amino Acid | QVQLQQSGGGVVQPGRSLRLSCAASGFTFSSYAMHWVRQAPGKGLEWVAVISYD |
| Sequence | GSNKYYADSVKGRFTISRDNSKNTLYLQMNSLRAEDTAVYYCAREVVGATLDYW |
| | GQGTLVTVSS (SEQ ID NO: 598) |

TABLE 66
Anti-CLL-1 Binder Clone 66

| Anti-CLL-1 Naïve | |
|---|---|
| Human Binder | |
| Clone AHP17220 | Sequence |
| V_{L} CDR 1 | ALPKQY (SEQ ID NO: 603) |
| V_{L} CDR 2 | KDS (SEQ ID NO: 604) |
| V_{L} CDR 3 | QSADSSGTC (SEQ ID NO: 605) |
| V_{H} CDR 1 | GDSVSSNSAA (SEQ ID NO: 607) |
| V_{H} CDR 2 | TYYRSKWYN (SEQ ID NO: 608) |
| V_{H} CDR 3 | ARAGGPHQTIL VAGRVRYYYYGMDV (SEQ ID NO: 609) |
| V_{L} Amino Acid | SYELTQLPSVSVSPGQTARITCSGDALPKQYAYWYQQKPGQAPVLVIYKDSERPSG |
| Sequence | IPERFSGSSSGTTVTLTISGVQAEDEADYYCQSADSSGTCFGGGTKLTVL (SEQ ID |
| | NO: 602) |
| V_{H} Amino Acid | QVQLQQSGPGLVKPSQTLSLTCDISGDSVSSNSAAWNWIRQSPSRGLEWLGRTYY |
| Sequence | RSKWYNDYAISVQSRITINPDTSKNQFSLQLKSVSPEDTAVYYCARAGGPHQTILV |
| | AGRVRYYYYGMDVWGQGTTVTVSS (SEQ ID NO: 606) |

TABLE 67
Anti-CLL-1 Binder Clone 67

| Anti-CLL-1 Naïve | |
|---|---|
| Human Binder | |
| Clone AHP17221 | Sequence |
| V_{L} CDR 1 | SSNIGGNT (SEQ ID NO: 611) |
| V_{L} CDR 2 | INS (SEQ ID NO: 612) |
| V_{L} CDR 3 | AAWDDSLNARV (SEQ ID NO: 613) |
| V_{H} CDR 1 | GGTFSSYA (SEQ ID NO: 615) |
| V_{H} CDR 2 | IIPILGIA (SEQ ID NO: 616) |
| V_{H} CDR 3 | AREHRDGYNFWSGVYYYGMDV (SEQ ID NO: 617) |
| V_{L} Amino Acid | SYVLTQPPSASGTPGQRVTISCSGSSSNIGGNTVIWYQQLPGSAPKLLIYINSQRPSG |
| Sequence | VPARFSGSKSGTSASLAITGLQSEDEADYYCAAWDDSLNARVFGGGTKVTVL |
| | (SEQ ID NO: 610) |
| V_{H} Amino Acid | QVQLVESGAEVKKPGSSVKVSCKASGGTFSSYAISWVRQAPGQGLEWMGGIIPILG |
| Sequence | IANYAQKFQGRVTITADESTSTAYMELSSLRSEDTAVYYCAREHRDGYNFWSGVY |
| | YYGMDVWDQGTTVTVSS (SEQ ID NO: 614) |

TABLE 68
Anti-CLL-1 Binder Clone 68

| Anti-CLL-1 Naïve | |
|---|---|
| Human Binder | |
| Clone AHP17222 | Sequence |
| V_{L} CDR 1 | NLGDKY (SEQ ID NO: 619) |
| V_{L} CDR 2 | QDN (SEQ ID NO: 620) |
| V_{L} CDR 3 | QAWDSSTAV (SEQ ID NO: 621) |
| V_{H} CDR 1 | GDSISSNNYY (SEQ ID NO: 623) |
| V_{H} CDR 2 | IYYSGST (SEQ ID NO: 624) |
| V_{H} CDR 3 | ARSARVSIPGRGGFDY (SEQ ID NO: 625) |
| V_{L} Amino Acid | SYELTQPPSVSVSPGQTASITCSGDNLGDKYACWYQQKPGQSPVLVMYQDNKRPS |
| Sequence | GIPERFSGSNSGNTATLTISGTQAMDEADYYCQAWDSSTAVFGGGTKVTVL (SEQ |
| | ID NO: 618) |
| V_{H} Amino Acid | QVQLQESGPGLVKPSETLSLTCTVSGDSISSNNYYWGWIRQPPGKGLDWIGFIYYS |
| Sequence | GSTYYNSSLKSRVTMSVDTSKNQFSLKLSSVTAADTAVYYCARSARVSIPGRGGF |
| | DYWGQGTLVTVSS (SEQ ID NO: 622) |

TABLE 69
Anti-CLL-1 Binder Clone 69

| Anti-CLL-1 Naïve | |
|---|---|
| Human Binder | |
| Clone AHP17224 | Sequence |
| V_{L} CDR 1 | TGVVTSGHY (SEQ ID NO: 627) |
| V_{L} CDR 2 | DTT (SEQ ID NO: 628) |
| V_{L} CDR 3 | FLSYSGARV (SEQ ID NO: 629) |
| V_{H} CDR 1 | GDSVSSNSAA (SEQ ID NO: 631) |
| V_{H} CDR 2 | TYYRSKWYN (SEQ ID NO: 632) |
| V_{H} CDR 3 | ARDGKKEKKYYYGSGSYRGAPNYFDY (SEQ ID NO: 633) |
| V_{L} Amino Acid | QTVVTQEPSLTVSPGGTVTLTCGSSTGVVTSGHYPYWFQQKPGQAPKTLIFDTTKK |
| Sequence | YSWTPARFSGSLLGGKAALTLSGAQPEDEADYYCFLSYSGARVFGGGTKLTVL |
| | (SEQ ID NO: 626) |
| V_{H} Amino Acid | QVQLQQSGPGLVKPSQTLSLTCAISGDSVSSNSAAWNWIRQSPSRGLEWLGRTYY |
| Sequence | RSKWYNDYAVSVKSRITINPDTSKNQFSLQLNSVTPEDTAVYYCARDGKKEKKYY |
| | YGSGSYRGAPNYFDYWGQGTLVTVSS (SEQ ID NO: 630) |

TABLE 70
Anti-CLL-1 Binder Clone 70

| Anti-CLL-1 Naïve | |
|---|---|
| Human Binder | |
| Clone AHP17225 | Sequence |
| V_{L} CDR 1 | LGIRND (SEQ ID NO: 635) |
| V_{L} CDR 2 | DAS (SEQ ID NO: 636) |
| V_{L} CDR 3 | LQHSSSLT (SEQ ID NO: 637) |
| V_{H} CDR 1 | GFSISTSGVG (SEQ ID NO: 639) |
| V_{H} CDR 2 | IYWDDDK (SEQ ID NO: 640) |
| V_{H} CDR 3 | AHRSRWASSWDLGWFDY (SEQ ID NO: 641) |
| V_{L} Amino Acid | DIQVTQSPSSLSASVGDRVTITCRASLGIRNDLGWCQQKPGKAPKRLIYDASSLQSG |
| Sequence | VPSRFSGSGSGTDFTLTISSLQPEDFANYNCLQHSSSLTFGGGTKVEIK (SEQ ID NO: |
| | 634) |
| V_{H} Amino Acid | RVTLKESGPPRVKPTQTLTLTCTLSGFSISTSGVGVGWIRQPPGKALEWLAFIYWD |
| Sequence | DDKRYNPSLKSRLTISKDTSKNQVVLTMTNMDPVDTATYFCAHRSRWASSWDLG |
| | WFDYWGQGTLVTVSS (SEQ ID NO: 638) |

TABLE 71
Anti-CLL-1 Binder Clone 71

| Anti-CLL-1 Naïve | |
|---|---|
| Human Binder | |
| Clone AHP17230 | Sequence |
| V_{L} CDR 1 | QSILYSSNNNNY (SEQ ID NO: 643) |
| V_{L} CDR 2 | WAS (SEQ ID NO: 644) |
| V_{L} CDR 3 | QHYLGTPPWT (SEQ ID NO: 645) |
| V_{H} CDR 1 | GGTFSSYA (SEQ ID NO: 647) |
| V_{H} CDR 2 | IIPIFGTA (SEQ ID NO: 648) |
| V_{H} CDR 3 | ASLGYCTNGVCWGPNLLGTPYMDV (SEQ ID NO: 649) |
| V_{L} Amino Acid | DIVMTQSPDSLAVSLGERATINCKSSQSILYSSNNNNYLAWYQQKPGQPPKLLIYW |
| Sequence | ASTRESGVPDRFSGSGSGTDFTLTISSLQAEDVAVYYCQHYLGTPPWTFGQGTKVD |
| | IK (SEQ ID NO: 642) |
| V_{H} Amino Acid | EVQLVQSGAEVKKPGSSVKVSCKASGGTFSSYAISWVRQAPGQGLEWMGGIIPIFG |
| Sequence | TANYAQKFQGRVTITADESTSTAYMELSSLRSEDTAVYYCASLGYCTNGVCWGPN |
| | LLGTPYMDVWGKGTTVTVSS (SEQ ID NO: 646) |

TABLE 72
Anti-CLL-1 Binder Clone 72

| Anti-CLL-1 Naïve | |
|---|---|
| Human Binder | |
| Clone AHP17235 | Sequence |
| V_{L} CDR 1 | SGSVSTNYY (SEQ ID NO: 651) |
| V_{L} CDR 2 | NTD (SEQ ID NO: 652) |
| V_{L} CDR 3 | LLYMGDGFWV (SEQ ID NO: 653) |
| V_{H} CDR 1 | GGSISSGDTY (SEQ ID NO: 655) |
| V_{H} CDR 2 | ISHSGST (SEQ ID NO: 656) |
| V_{H} CDR 3 | ARALYYSGGWGYFDY (SEQ ID NO: 657) |
| V_{L} Amino Acid | QSVVTQEPSLSVSPGGTVTLTCGLSSGSVSTNYYASWYQKTPGQAPRTLIYNTDSR |
| Sequence | SSGVPDRFSGSILGNTAALTITGAQADDESDYYCLLYMGDGFWVFGGGTQLIIL |
| | (SEQ ID NO: 650) |
| V_{H} Amino Acid | QVQLQESGPGLVKPSQTLSLTCTVSGGSISSGDTYWSWIRQHPGMGLVWIGYISHS |
| Sequence | GSTYYNPSLKTRVTISVDTSKNQFSLNLSSVTAADTAVYYCARALYYSGGWGYFD |
| | YWGQGTLVTVSS (SEQ ID NO: 654) |

TABLE 73
Anti-CLL-1 Binder Clone 73

| Anti-CLL-1 Naïve | |
|---|---|
| Human Binder | |
| Clone AHP17236 | Sequence |
| V_{L} CDR 1 | TGAVTNAYH (SEQ ID NO: 659) |
| V_{L} CDR 2 | STN (SEQ ID NO: 660) |
| V_{L} CDR 3 | LLYFDDPQPLWV (SEQ ID NO: 661) |
| V_{H} CDR 1 | GGSISSYY (SEQ ID NO: 663) |
| V_{H} CDR 2 | IYYSGST (SEQ ID NO: 664) |
| V_{H} CDR 3 | AREARPDSYYYYYMDV (SEQ ID NO: 665) |
| V_{L} Amino Acid | QAVVTQEPSLTVSPGGTVTLTCASGTGAVTNAYHPNWFQQKPGQAPRPLIYSTNN |
| Sequence | KYSWTPARFSGSLLGDKAVLTLSGVQPEDEAEYYCLLYFDDPQPLWVFGGGTKLT |
| | VL (SEQ ID NO: 658) |
| V_{H} Amino Acid | QVQLQESGPGLVKPSETLSLTCTVSGGSISSYYWSWIRQPPGKGLEWIGYIYYSGST |
| Sequence | NYNPSLKSRVTISVDTSKNQFSLKLSSVTAADTAVYYCAREARPDSYYYYYMDV |
| | WGKGTTVTVSS (SEQ ID NO: 662) |

TABLE 74
Anti-CLL-1 Binder Clone 74

| Anti-CLL-1 Naïve | |
|---|---|
| Human Binder | |
| Clone AHP17237 | Sequence |
| V_{L} CDR 1 | QSLLHSNGYNY (SEQ ID NO: 667) |
| V_{L} CDR 2 | LGS (SEQ ID NO: 668) |
| V_{L} CDR 3 | MQALQTPPWT (SEQ ID NO: 669) |
| V_{H} CDR 1 | GFTFSSYW (SEQ ID NO: 671) |
| V_{H} CDR 2 | IKQDGSEK (SEQ ID NO: 672) |
| V_{H} CDR 3 | AREGLGYYYDSSGSIDYFDY (SEQ ID NO: 673) |
| V_{L} Amino Acid | DIVMTQTPLSLPVTPGEPASISCRSSQSLLHSNGYNYLDWYLQKPGQSPQLLIYLGS |
| Sequence | NRASGVPDRFSGSGSGTDFTLKISRVEAEDVGVYYCMQALQTPPWTFGQGTKLEI |
| | K (SEQ ID NO: 666) |
| V_{H} Amino Acid | ETQLVESGGGLVQPGGSLRLSCAASGFTFSSYWMSWVRQAPGKGLEWVANIKQD |
| Sequence | GSEKYYVDSVKGRFTISRDNAKNSLYLQMNSLRAEDTAVYYCAREGLGYYYDSS |
| | GSIDYFDYWGQGTLVTVSS (SEQ ID NO: 670) |

TABLE 75
Anti-CLL-1 Binder Clone 75

| Anti-CLL-1 Naïve | |
|---|---|
| Human Binder | |
| Clone AHP17238 | Sequence |
| V_{L} CDR 1 | SGNIATNY (SEQ ID NO: 675) |
| V_{L} CDR 2 | EDN (SEQ ID NO: 676) |
| V_{L} CDR 3 | QSYDGSSVI (SEQ ID NO: 677) |
| V_{H} CDR 1 | GYTFTGYY (SEQ ID NO: 679) |
| V_{H} CDR 2 | INPNSGGT (SEQ ID NO: 680) |
| V_{H} CDR 3 | AEGHSRRWSPGDY (SEQ ID NO: 681) |
| V_{L} Amino Acid | NFMLTQPHSVSESPGKTVTISCARSSGNIATNYVQWYQQRPGSSPTAVIFEDNQRPS |
| Sequence | GVPDRFSGSIDSSSNSASLTISGLQTADEADYYCQSYDGSSVIFGGGTQLIIL (SEQ |
| | ID NO: 674) |
| V_{H} Amino Acid | EVQLVQSGAEVKKPGASVKVSCKASGYTFTGYYMHWVRQAPGQGLEWMGWINP |
| Sequence | NSGGTNYAQKFQGRVTMTRDTSLNTVYMELRALRVDDTAVYYCAEGHSRRWSP |
| | GDYWGQGSLVTVSS (SEQ ID NO: 678) |

TABLE 76
Anti-CLL-1 Binder Clone 76

| Anti-CLL-1 Naïve | |
|---|---|
| Human Binder | |
| Clone AHP17239 | Sequence |
| V_{L} CDR 1 | TGVVTSGHY (SEQ ID NO: 683) |
| V_{L} CDR 2 | DTS (SEQ ID NO: 684) |
| V_{L} CDR 3 | LLSFGGAPNWV (SEQ ID NO: 685) |
| V_{H} CDR 1 | GFSLSNARMG (SEQ ID NO: 687) |
| V_{H} CDR 2 | IFSNDEK (SEQ ID NO: 688) |
| V_{H} CDR 3 | ARVPRGVIIDYYYYGMDV (SEQ ID NO: |
| | 689) |
| V_{L} Amino Acid | QSVVTQEPSLTVSPGGTVTLTCGSSTGVVTS |
| Sequence | GHYPYWFQQKPGQAPTTLIYDTSNKHSWTPA |
| | RFSGSLLGGKAVLTLSGAQPEDEAEYYCLLS |
| | FGGAPNWVFGGGTKLTVL |
| | (SEQ ID NO: 682) |
| V_{H} Amino Acid | RVTLKESGPVLVKPTETLTLTCTVSGFSLSN |
| Sequence | ARMGVSWIRQPPGKALEWLAHIFSNDEKSYS |
| | TSLKSRLTISKDTSKSQVVLTMTNMDPVDTA |
| | TYYCARVPRGVIIDYYYYGMDVWGQGTTVTV |
| | SS (SEQ ID NO: 686) |

TABLE 77
Anti-CLL-1 Binder Clone 77

| Anti-CLL-1 Naïve | |
|---|---|
| Human Binder | |
| Clone AHP17240 | Sequence |
| V_{L} CDR 1 | SSDVGSYDY (SEQ ID NO: 691) |
| V_{L} CDR 2 | NVN (SEQ ID NO: 692) |
| V_{L} CDR 3 | CSYTSSATYV (SEQ ID NO: 693) |
| V_{H} CDR 1 | NYPFTTYG (SEQ ID NO: 695) |
| V_{H} CDR 2 | ISTYKGNT (SEQ ID NO: 696) |
| V_{H} CDR 3 | ARGRTTLLHGEYLV (SEQ ID NO: 697) |
| V_{L} Amino Acid | QSALTQPPSVSGSPGQSVTISCTGTSSDVGS |
| Sequence | YDYVSWYQQHPGTVPKPMIYNVNTQPSGVPD |
| | RFSGSKSGNTASMTISGLQAEDEADYQCCSY |
| | TSSATYVFGTGTKLTVL(SEQ ID NO: |
| | 690) |
| V_{H} Amino Acid | ETQLVESGPEVKRPGASVKVSCKASNYPFTT |
| Sequence | YGITWVRQAPGQGLEWMGWISTYKGNTNYAQ |
| | RLQGRVTMTVETFTSTAYMELKNLTSDDTAI |
| | YYCARGRTTLLHGEYLVWGQGPLVTVSS |
| | (SEQ ID NO: 694) |

TABLE 78
Anti-CLL-1 Binder Clone 78

| Anti-CLL-1 Naïve | |
|---|---|
| Human Binder | |
| Clone AHP17241 | Sequence |
| V_{L} CDR 1 | SSDVGADNY (SEQ ID NO: 699) |
| V_{L} CDR 2 | DLS (SEQ ID NO: 700) |
| V_{L} CDR 3 | SSYTSSSTYV (SEQ ID NO: 701) |
| V_{H} CDR 1 | GGSFSGYY (SEQ ID NO: 703) |
| V_{H} CDR 2 | INHSGST (SEQ ID NO: 704) |
| V_{H} CDR 3 | ARVSTWVRGAHNSYYYYGMDV (SEQ ID |
| | NO: 705) |
| V_{L} Amino Acid | QSALTQPASVSGSPGQSITISCAGTSSDVGA |
| Sequence | DNYVSWYQQHPGKAPKLMIYDLSKRPSGVSN |
| | RFSGSKSGNTASLTISGLQADDEADYYCSSY |
| | TSSSTYVFGTGTKVTVL (SEQ ID NO: |
| | 698) |
| V_{H} Amino Acid | QVQLQQWGAGLLKPSETLSLTCAVYGGSFSG |
| Sequence | YYWSWIRQPPGKGLEWIGEINHSGSTNYNPS |
| | LKSRVTISVDTSKNQFSLKLSSVTAADTAVY |
| | YCARVSTWVRGAHNSYYYYGMDVWGQGTTVT |
| | VSS (SEQ ID NO: 702) |

TABLE 79
Anti-CLL-1 Binder Clone 79

| Anti-CLL-1 Naïve | |
|---|---|
| Human Binder | |
| Clone AHP17243 | Sequence |
| V_{L} CDR 1 | SSDVGGYNY (SEQ ID NO: 707) |
| V_{L} CDR 2 | GVT (SEQ ID NO: 708) |
| V_{L} CDR 3 | SSFTTGSFV (SEQ ID NO: 709) |
| V_{H} CDR 1 | GGTFSSYA (SEQ ID NO: 711) |
| V_{H} CDR 2 | IIPIFGTA (SEQ ID NO: 712) |
| V_{H} CDR 3 | ASPGRERAAAGPVAPSLPGPYYYYYMDV |
| | (SEQ ID NO: 713) |
| V_{L} Amino Acid | QSALTQPASVSGSPGQSITISCTGTSSDVGG |
| Sequence | YNYVSWYQQHPGKVPKLMIYGVTNRPSGVSN |
| | RFSGSKSGNTASLTISGLQADDEADYYCSSF |
| | TTGSFVFGTGTKVTVL (SEQ ID NO: |
| | 706) |
| V_{H} Amino Acid | EVHLVESGAEVKKPGSSVKVSCKASGGTFSS |
| Sequence | YAISWVRQAPGQGLEWMGGIIPIFGTANYAQ |
| | KFQGRVTITADESTSTAYMELSSLRSEDTAV |
| | YYCASPGRERAAAGPVAPSLPGPYYYYYMDV |
| | WGKGTTVTVSS (SEQ ID NO: 710) |

TABLE 80
Anti-CLL-1 Binder Clone 80

| Anti-CLL-1 Naïve | |
|---|---|
| Llama Binder Clone | |
| AHP17245 | Sequence |
| V_{H} Amino Acid | QVQRQESGGGLVQPGGSLRLSCSVSGNID |
| Sequence | RFYAMGWYRQAPGKQRELVAQLTNNEITT |
| | YGDSVEGQFSISGDFDANTVYLQMDSLKP |
| | EDTAAYYCYYRIMSLTHTGPRYSWGQGTQ |
| | VTVSS (SEQ ID NO: 714) |
| V_{H} CDR 1 | GNIDRFYA (SEQ ID NO: 715) |
| V_{H} CDR 2 | LTNNEIT (SEQ ID NO: 716) |
| V_{H} CDR 3 | YYRIMSLTHTGPRYS (SEQ ID NO: |
| | 717) |

TABLE 81
Anti-CLL-1 Binder Clone 81

| Anti-CLL-1 Naïve | |
|---|---|
| Llama Binder Clone | |
| AHP17246 | Sequence |
| V_{H} Amino Acid | EVQLVESGGGLVQPGGSLTLACAASGFTF |
| Sequence | SSATQTWVRQGPGKGLEWVSDINRGDGNG |
| | YYADSVKGRFTISRDNAKNTLYLEMNNLK |
| | PEDTAVYYCGKGFGAWGQGTQVTVSS |
| | (SEQ ID NO: 718) |
| V_{H} CDR 1 | GFTFSSAT (SEQ ID NO: 719) |
| V_{H} CDR 2 | INRGDGNG (SEQ ID NO: 720) |
| V_{H} CDR 3 | GKGFGA (SEQ ID NO: 721) |

TABLE 82
Anti-CLL-1 Binder Clone 82

| Anti-CLL-1 Naïve | |
|---|---|
| Llama Binder Clone | |
| AHP17247 | Sequence |
| V_{H} Amino Acid | QVKLEESGGGLVQPGGSLRLSCAISGSVF |
| Sequence | SIDALGWYRQAPGQQRELVAHISRTGSTH |
| | YSDFVLGRFTISRDNARNSVYLQMNNLKP |
| | EDTAVYFCNADLGPRADPVWGRGTSVTVS |
| | S (SEQ ID NO: 722) |
| V_{H} CDR 1 | GSVFSIDA (SEQ ID NO: 723) |
| V_{H} CDR 2 | ISRTGST (SEQ ID NO: 724) |
| V_{H} CDR 3 | NADLGPRADPV (SEQ ID NO: 725) |

TABLE 83
Anti-CLL-1 Binder Clone 83

| Anti-CLL-1 Naïve | |
|---|---|
| Llama Binder Clone | |
| AHP17248 | Sequence |
| V_{H} Amino Acid | QVKLEESGGGLVQAGGSLRLSCAASPRTS |
| Sequence | GGYTMAWFRQASGKERDFVARTGWISDST |
| | DYADSVKSRFTISRDSAKDTLYLQMNSLK |
| | PEDTAVYYCAASRKYYSSNSYYREVPTYD |
| | YWGQGTQVTVSS (SEQ ID NO: 726) |
| V_{H} CDR 1 | PRTSGGYT (SEQ ID NO: 727) |
| V_{H} CDR 2 | TGWISDST (SEQ ID NO: 728) |
| V_{H} CDR 3 | AASRKYYSSNSYYREVPTYDY (SEQ ID |
| | NO: 729) |

TABLE 84
Anti-CLL-1 Binder Clone 84

| Anti-CLL-1 Naïve | |
|---|---|
| Llama Binder Clone | |
| AHP17252 | Sequence |
| V_{H} Amino Acid | QVQLVESGGGLGQPGGSLRLSCVASGGTF |
| Sequence | AREPIHWYRQTPGNQRELVASIFLTRSPH |
| | YADFVKGRFTVSRDVAANTAYLQMNSLKP |
| | EDTAIYYCALEYQLRHYWGQGTQVTVSS |
| | (SEQ ID NO: 730) |
| V_{H} CDR 1 | GGTFAREP (SEQ ID NO: 731) |
| V_{H} CDR 2 | IFLTRSP (SEQ ID NO: 732) |
| V_{H} CDR 3 | ALEYQLRHY (SEQ ID NO: 733) |

TABLE 85
Anti-CLL-1 Binder Clone 85

| Anti-CLL-1 Naïve | |
|---|---|
| Llama Binder Clone | |
| AHP17253 | Sequence |
| V_{H} Amino Acid | EVQLVESGGGSVQAGDSLRLSCAASGGTF |
| Sequence | RSYTMGWFRQTPGKEREFVAALGGSIWPA |
| | PYYTDSVKGRFNISGDNAKSTVYLQMNSL |
| | KQEDTAVYYCAAGGSDYSLRLSPSDYDYW |
| | GQGTQVTVSP (SEQ ID NO: 734) |
| V_{H} CDR 1 | GGTFRSYT (SEQ ID NO: 735) |
| V_{H} CDR 2 | LGGSIWPA (SEQ ID NO: 736) |
| V_{H} CDR 3 | CAAGGSDYSLRLSPSDYDY (SEQ ID |
| | NO: 737) |

TABLE 86
Anti-CLL-1 Binder Clone 86

| Anti-CLL-1 Naïve | |
|---|---|
| Llama Binder Clone | |
| AHP17254 | Sequence |
| V_{H} Amino Acid | AVQLVDSGGGRVQPGGSLRLSCAASGNTL |
| Sequence | ELYAIGWFRQGPGKEREGISCITSYDGDT |
| | YYTDSVKGRFTIPRDNARNSVYLHMNNLK |
| | PEDTGVYYCAAGTTFTTMQSVCIRTGWYS |
| | TWGQGTQVTVSS (SEQ ID NO: 738) |
| V_{H} CDR 1 | GNTLELYA (SEQ ID NO: 739) |
| V_{H} CDR 2 | ITSYDGDT (SEQ ID NO: 740) |
| V_{H} CDR 3 | AAGTTFTTMQSVCIRTGWYST (SEQ ID |
| | NO: 741) |

TABLE 87
Anti-CLL-1 Binder Clone 87

| Anti-CLL-1 Naïve | |
|---|---|
| Llama Binder Clone | |
| AHP17255 | Sequence |
| V_{H} Amino Acid | DVQLVESGGGLVQPGGSLRLSCTASGSTF |
| Sequence | SDSWMYWVRQAPGKGLELVSPINSYGGAT |
| | YYADSVKGRFTISRDNAKNTLYLQMNSLK |
| | PEDTALYYCATHDSDQGIYQFNYWGQGTQ |
| | VTVSS (SEQ ID NO: 742) |
| V_{H} CDR 1 | GSTFSDSW (SEQ ID NO: 743) |
| V_{H} CDR 2 | INSYGGAT (SEQ ID NO: 744) |
| V_{H} CDR 3 | ATHDSDQGIYQFNY (SEQ ID NO: |
| | 745) |

TABLE 88
Anti-CLL-1 Binder Clone 88

| Anti-CLL-1 Naïve | |
|---|---|
| Llama Binder Clone | |
| AHP17256 | Sequence |
| V_{H} Amino Acid | AVQLVDSGGGLVQPGGSLRLSCAASGFTF |
| Sequence | DDYAMSWVRQAPGKGLEWVSAISWNDGRT |
| | YYAESMKGRFTISRDNAKNTLYLQMNSLK |
| | SEDTAVYYCAKAYEDLETIPNHQGFGSWG |
| | QGIQVTVSS (SEQ ID NO: 746) |
| V_{H} CDR 1 | GFTFDDYA (SEQ ID NO: 747) |
| V_{H} CDR 2 | ISWNDGRT (SEQ ID NO: 748) |
| V_{H} CDR 3 | AKAYEDLETIPNHQGFGS (SEQ ID |
| | NO: 749) |

TABLE 89
Anti-CLL-1 Binder Clone 89

| Anti-CLL-1 Naïve | |
|---|---|
| Llama Binder Clone | |
| AHP17257 | Sequence |
| V_{H} Amino Acid | QVKLEESGGGLVQPGGSLRLSCAASGFTF |
| Sequence | SSSAMSWVRQAPGKGLEWVSSIYNYGSNT |
| | YYADSVKGRLTISTDNAKNTLYLQMNSLK |
| | SGDTAVYYCAKEAPLYYSDYDSGTSFGSW |
| | GQGTQVTVSS (SEQ ID NO: 750) |
| V_{H} CDR 1 | GFTFSSSA (SEQ ID NO: 751) |
| V_{H} CDR 2 | IYNYGSNT (SEQ ID NO: 752) |
| V_{H} CDR 3 | AKEAPLYYSDYDSGTSFGS (SEQ ID |
| | NO: 753) |

TABLE 90
Anti-CLL-1 Binder Clone 90

| Anti-CLL-1 Naïve | |
|---|---|
| Llama Binder Clone | |
| AHP17258 | Sequence |
| V_{H} Amino Acid | QVKLEESGGGLVQPGGSLRLSCAASGFTF |
| Sequence | STSTMSWVRQAPGKGLEWVSSIYSYISNT |
| | YYADSVKGRFTISTDNAKNTLYLRMNSLK |
| | SEDTAVYYCAKTELSSFGLGLNLGYDYWG |
| | QGTQVTVSS (SEQ ID NO: 754) |
| V_{H} CDR 1 | GFTFSTST (SEQ ID NO: 755) |
| V_{H} CDR 2 | IYSYISNT (SEQ ID NO: 756) |
| V_{H} CDR 3 | AKTELSSFGLGLNLGYDY (SEQ ID |
| | NO: 757) |

TABLE 91
Anti-CLL-1 Binder Clone 91

| Anti-CLL-1 Naive | |
|---|---|
| Llama Binder Clone | |
| AHP17259 | Sequence |
| V_{H} Amino Acid | AVQLVDSGGGLVKPGGSLRLSCVASGSIFGMNAMGWYRQVLGQERELVAAITKD |
| Sequence | GETMYAESVKGRFTISGDKAKHTVSLQMNNLNVEDTAVYYCKAAVVNLFGGSTY |
| | EYDYWGQGTQVTVSS (SEQ ID NO: 758) |
| V_{H} CDR 1 | GSIFGMNA (SEQ ID NO: 759) |
| V_{H} CDR 2 | ITKDGET (SEQ ID NO: 760) |
| V_{H} CDR 3 | KAAVVNLFGGSTYEYDY (SEQ ID NO: 761) |

TABLE 92
Anti-CLL-1 Binder Clone 92

| Anti-CLL-1 Naive | |
|---|---|
| Llama Binder | |
| AHP17260 | Sequence |
| V_{H} Amino Acid | QVQLVESGGGSVQPGGSLRLSCAVDAFTFESFHIGWYREAPGKEREGVSCMANRG |
| Sequence | GSTWTADSVQGRFTISRDNTKRTVYLQMDSLRLEDTATYYCATCGSDSPLGYSGK |
| | GTLVTVSS (SEQ ID NO: 762) |
| V_{H} CDR 1 | AFTFESFH (SEQ ID NO: 763) |
| V_{H} CDR 2 | MANRGGST (SEQ ID NO: 764) |
| V_{H} CDR 3 | ATCGSDSPLGY (SEQ ID NO: 765) |

TABLE 93
Anti-CLL-1 Binder Clone 93

| Anti-CLL-1 Naive | |
|---|---|
| Llama Binder Clone | |
| AHP17261 | Sequence |
| V_{H} Amino Acid | AVQLVDSGGGLAQPGGSLRLSCAASGFTFTSYWMHWVRQAPGKGLEWVSVITPD |
| Sequence | GASTRYAESVKGRFTISRDNAKNTLYLQMNSLKPEDTAVYYCISPPAMESWGKGT |
| | LVTVSS (SEQ ID NO: 766) |
| V_{H} CDR 1 | GFTFTSYW (SEQ ID NO: 767) |
| V_{H} CDR 2 | ITPDGAST (SEQ ID NO: 768) |
| V_{H} CDR 3 | ISPPAMES (SEQ ID NO: 769) |

TABLE 94
Anti-CLL-1 Binder Clone 94

| Anti-CLL-1 Binder | |
|---|---|
| Clone 94 | Sequence |
| V_{H} CDR 1 | AYDMD (SEQ ID NO: 770) |
| V_{H} CDR 2 | KISHDGSYSWYADSVKG (SEQ ID NO: 771) |
| V_{H} CDR 3 | GEGGSERADY (SEQ ID NO: 772) |
| V_{H} Nucleotide | CAGGTGCAGCTGGTGCAGTCTGGGGGAGGCTTGGTACAGCCTGGAGGGTCCCT |
| Sequence | GAGACTCTCCTGTGCAGCCTCTTATTTCGATTTCGCTGCTTATGATATGGACTG |
| | GGTCCGCCAGGCTCCAGGACAACGCCTTGAGTGGGTGGCAAAAATTTCACATG |
| | ATGGAAGTTATAGTTGGTATGCAGACTCTGTGAAGGGCCGATTCACCATCTCC |
| | AGAGACAATTCCAAGAACACGCTGTATCTGCAAATGAACACCCTGAGAGCCGA |
| | GGACACGGCCGTATATTACTGTGTGAGAGGGGAAGGTGGCAGTGAACGCGCT |
| | GATTACTGGGGCCGGGGAACCCTGGTCACCGTCTCTTCA (SEQ ID NO: 773) |
| V_{H} Amino Acid | QVQLVQSGGGLVQPGGSLRLSCAASYFDFAAYDMDWVRQAPGQRLEWVAKISH |
| Sequence | DGSYSWYADSVKGRFTISRDNSKNTLYLQMNTLRAEDTAVYYCVRGEGGSERAD |
| | YWGRGTLVTVSS (SEQ ID NO: 774) |

TABLE 95
Anti-CLL-1 Binder Clone 95

| Anti-CLL-1 Binder | |
|---|---|
| Clone 95 | Sequence |
| V_{H} CDR 1 | DSSMD (SEQ ID NO: 775) |
| V_{H} CDR 2 | SISYFGSTYYNPSLKS (SEQ ID NO: 776) |
| V_{H} CDR 3 | SALYGDAAFDV (SEQ ID NO: 777) |
| V_{H} Nucleotide | CAGGTGCAGCTGGTGCAGTCTGGGGGAGGCTTGGTACAGCCTGGAGGGTCCCT |
| Sequence | GAGACTCTCCTGTGCAGCCTCTGCTTTCGATTTCGATGATTCTTCTATGGACTG |
| | GGTCCGCCAGGCTCCAGGGAAGGCCCTGGAGTGGATTGGGAGTATCAGTTATT |
| | TTGGGAGTACCTACTACAACCCGTCCCTCAAGAGTCGAGTCACCATCTCCAGA |
| | GACAATTCCAAGAACACGCTGTATCTGCAAATGAACACCCTGAGAGCCGAGGA |
| | CACAGCCACATATTACTGTACAAGATCGGCCTTATACGGAGACGCAGCCTTCG |
| | ATGTCTGGGCCCAAGGGACAACGGTCACTGTCTCT (SEQ ID NO: 778) |
| V_{H} Amino Acid | QVQLVQSGGGLVQPGGSLRLSCAASAFDFDDSSMDWVRQAPGKALEWIGSISYFG |
| Sequence | STYYNPSLKSRVTISRDNSKNTLYLQMNTLRAEDTATYYCTRSALYGDAAFDVW |
| | AQGTTVTVS (SEQ ID NO: 779) |

TABLE 96
Anti-CLL-1 Binder Clone 96

| Anti-CLL-1 Binder | |
|---|---|
| Clone 96 | Sequence |
| V_{H} CDR 1 | YYEMS (SEQ ID NO: 780) |
| V_{H} CDR 2 | LMSGNGGSTYYADSVKG (SEQ ID NO: 781) |
| V_{H} CDR 3 | HSSTWFS (SEQ ID NO: 782) |
| V_{H} Nucleotide | CAGGTGCAGCTGGTGCAGTCTGGGGGAGGCTTGGTACAGCCTGGAGGGTCCCT |
| Sequence | GAGACTCTCCTGTGCAGCCTCTTATTTCGATTTCTCTTATTATGAAATGAGCTGG |
| | GTCCGCCAGGCTCCAGGGAAGGGGCTGGAGTGGGTCTCACTTATGAGTGGTAA |
| | TGGTGGTAGCACATACTACGCAGACTCAGTGAAGGGCCGATTCACCATCTCCA |
| | GAGACAATTCTAAGAACACGCTGTATCTGCAAATGAATACCCTGAGAGCCGAG |
| | GACACAGCCACGTATTACTGTGTGGCCCACAGCAGCACCTGGTTTTCTTGGGGC |
| | CAAGGAACCCTGGTCACCGTCTCCTCA (SEQ ID NO: 783) |
| V_{H} Amino Acid | QVQLVQSGGGLVQPGGSLRLSCAASYFDFSYYEMSWVRQAPGKGLEWVSLMSGN |
| Sequence | GGSTYYADSVKGRFTISRDNSKNTLYLQMNTLRAEDTATYYCVAHSSTWFSWGQ |
| | GTLVTVSS (SEQ ID NO: 784) |

TABLE 97
Anti-CLL-1 Binder Clone 97

| Anti-CLL-1 Binder | |
|---|---|
| Clone 97 | Sequence |
| V_{H} CDR 1 | DDSMD (SEQ ID NO: 785) |
| V_{H} CDR 2 | SVLHDGATYYNPSLKS (SEQ ID NO: 786) |
| V_{H} CDR 3 | SALYGDAAFDV (SEQ ID NO: 777) |
| V_{H} Nucleotide | CAGGTGCAGCTGGTGCAGTCTGGGGGAGGCTTGGTACAGCCTGGAGGGTCCCT |
| Sequence | GAGACTCTCCTGTGCAGCCTCTGATTTCGATTTCGATGATGATTCTATGGACTG |
| | GGTCCGCCAGGCTCCAGGGAAGGGGCTAGAGTGGATTGGGAGTGTCTTACATG |
| | ACGGAGCCACCTATTACAACCCGTCCCTCAAGAGTCGAGTCACCATCTCCAGA |
| | GACAATTCCAAGAACACGCTGTATCTGCAAATGAACACCCTGAGAGCCGAGGA |
| | CACAGCCACATATTACTGTACAAGATCGGCCTTATACGGGGACGCGGCCTTCG |
| | ATGTCTGGGCCCAAGGGACAATGGTCACCGTCTCTTCA (SEQ ID NO: 787) |
| VH Amino Acid | QVQLVQSGGGLVQPGGSLRLSCAASDFDFDDDSMDWVRQAPGKGLEWIGSVLHD |
| Sequence | GATYYNPSLKSRVTISRDNSKNTLYLQMNTLRAEDTATYYCTRSALYGDAAFDV |
| | WAQGTMVTVSS (SEQ ID NO: 788) |

TABLE 98
Anti-CLL-1 Binder Clone 98

| Anti-CLL-1 Binder | |
|---|---|
| Clone 98 | Sequence |
| V_{H} CDR 1 | DYEMS (SEQ ID NO: 789) |
| V_{H} CDR 2 | LISSDGTTYYADSVKG (SEQ ID NO: 790) |
| V_{H} CDR 3 | TTDSWELDL (SEQ ID NO: 791) |
| V_{H} Nucleotide | CAGGTGCAGCTGGTGCAGTCTGGGGGAGGCTTGGTACAGCCTGGAGGGTCCCT |
| Sequence | GAGACTCTCCTGTGCAGCCTCTGATTTCTATTTCGATGATTATGAAATGAGCTG |
| | GGTCCGCCAGGCTCCAGGACAACGCCTTGAGTGGGTCTCACTTATCTCTAGCGA |
| | CGGTACCACATACTACGCAGACTCTGTGAAGGGCCGATTCACCATCTCCAGAG |
| | ACAATTCCAAGAACACGCTGTATCTGCAAATGAACACCCTGAGAGCCGAGGAC |
| | ACGGCTTTGTATTACTGTGTGCGAACCACGGACTCCTGGGAGCTCGATCTCTGG |
| | GGCCGTGGCACCCTGGTCACCGTCTCCTCA (SEQ ID NO: 792) |
| V_{H} Amino Acid | QVQLVQSGGGLVQPGGSLRLSCAASDFYFDDYEMSWVRQAPGQRLEWVSLISSD |
| Sequence | GTTYYADSVKGRFTISRDNSKNTLYLQMNTLRAEDTALYYCVRTTDSWELDLWG |
| | RGTLVTVSS (SEQ ID NO: 793) |

TABLE 99
Anti-CLL-1 Binder Clone 99

| Anti-CLL-1 Binder | |
|---|---|
| Clone 99 | Sequence |
| V_{H} CDR 1 | SYEMS (SEQ ID NO: 794) |
| V_{H} CDR 2 | YIGPIGTDIGYADSVKG (SEQ ID NO: 795) |
| V_{H} CDR 3 | DPYYDSTGLTSY (SEQ ID NO: 796) |
| V_{H} Nucleotide | CAGGTGCAGCTGGTGCAGTCTGGGGGAGGCTTGGTACAGCCTGGAGGGTCCCT |
| Sequence | GAGACTCTCCTGTGCAGCCTCTGCTTTCGATTTCTATTCTTATGAAATGAGCTG |
| | GGTCCGCCAGGCTCCAGGGAAGGGGCTAGAGTGGGTTTCATACATTGGTCCCA |
| | TTGGTACTGACATAGGCTACGCAGACTCCGTGAAGGGCCGATTCACCATCTCC |
| | AGAGACAATTCCAAGAACACGCTGTATCTGCAAATGAACACCCTGAGAGCCGA |
| | GGACACAGCCATGTATTACTGTGCGAAAGACCCTTACTATGATAGTACTGGGTT |
| | AACGAGCTACTGGGGCCAGGGAACCCTGGTCACCGTCTCCTCA (SEQ ID NO: |
| | 797) |
| V_{H} Amino Acid | QVQLVQSGGGLVQPGGSLRLSCAASAFDFYSYEMSWVRQAPGKGLEWVSYIGPIG |
| Sequence | TDIGYADSVKGRFTISRDNSKNTLYLQMNTLRAEDTAMYYCAKDPYYDSTGLTSY |
| | WGQGTLVTVSS (SEQ ID NO: 798) |

TABLE 100
Anti-CLL-1 Binder Clone 100

| Anti-CLL-1 Binder | |
|---|---|
| Clone 100 | Sequence |
| V_{H} CDR 1 | DDSMD (SEQ ID NO: 785) |
| V_{H} CDR 2 | SVSYSGQTFYNPSLKS (SEQ ID NO: 799) |
| V_{H} CDR 3 | SALYGDAAFDV (SEQ ID NO: 777) |
| V_{H} Nucleotide | CAGGTGCAGCTGGTGCAGTCTGGGGGAGGCTTGGTACAGCCTGGAGGGTCCCT |
| Sequence | GAGACTCTCCTGTGCAGCCTCTGATTTCGATTTCGATGATGATTCTATGGACTG |
| | GGTCCGCCAGGCTCCAGGGAAGGCCCTGGAGTGGATTGGGAGTGTCTCTTATA |
| | GTGGACAAACCTTCTACAACCCGTCCCTCAAGAGTCGAGTCACCATCTCCAGA |
| | GACAATTCCAAGAACACGCTGTATCTGCAAATGAACACCCTGAGAGCCGAGGA |
| | CACAGCCATGTATTACTGTACAAGATCGGCCTTATACGGAGACGCGGCCTTCG |
| | ATGTCTGGGCCCAAGGGACAATGGTCACCGTCTCCTCA (SEQ ID NO: 800) |
| V_{H} Amino Acid | QVQLVQSGGGLVQPGGSLRLSCAASDFDFDDDSMDWVRQAPGKALEWIGSVSYS |
| Sequence | GQTFYNPSLKSRVTISRDNSKNTLYLQMNTLRAEDTAMYYCTRSALYGDAAFDV |
| | WAQGTMVTVSS (SEQ ID NO: 801) |

TABLE 101
Anti-CLL-1 Binder Clone 101

| Anti-CLL-1 Binder | |
|---|---|
| Clone 101 | Sequence |
| V_{H} CDR 1 | DDSMD (SEQ ID NO: 785) |
| V_{H} CDR 2 | SIFYSGTTYYNPSLKS (SEQ ID NO: 802) |
| V_{H} CDR 3 | SALYGDAAFDV (SEQ ID NO: 777) |
| V_{H} Nucleotide | CAGGTGCAGCTGGTGCAGTCTGGGGGAGGCTTGGTACAGCCTGGAGGGTCCCT |
| Sequence | GAGACTCTCCTGTGCAGCCTCTGATTTCGATTTCGCTGATGATTCTATGGACTG |
| | GGTCCGCCAGGCTCCAGGGAAGGCCCTGGAGTGGATTGGGAGTATCTTTTATA |
| | GTGGGACCACCTACTACAACCCGTCCCTCAAGAGTCGAGTCACCATCTCCAGA |
| | GACAATTCCAAGAACACGCTGTATCTGCAAATGAACACCCTGAGAGCCGAGGA |
| | CACAGCCGTATATTACTGTACCAGATCGGCCTTATACGGAGACGCGGCCTTCG |
| | ATGTCTGGGCCCAAGGGACAATGGTCACCGTCTCCTCA (SEQ ID NO: 803) |
| V_{H} Amino Acid | QVQLVQSGGGLVQPGGSLRLSCAASDFDFADDSMDWVRQAPGKALEWIGSIFYSG |
| Sequence | TTYYNPSLKSRVTISRDNSKNTLYLQMNTLRAEDTAVYYCTRSALYGDAAFDVW |
| | AQGTMVTVSS (SEQ ID NO: 804) |

TABLE 102
Anti-CLL-1 Binder Clone 102

| Anti-CLL-1 Binder | |
|---|---|
| Clone 102 | Sequence |
| V_{H} CDR 1 | DYEMS (SEQ ID NO: 789) |
| V_{H} CDR 2 | VVSKDGNELYYADSVKG (SEQ ID NO: 805) |
| V_{H} CDR 3 | DPYDFWSGYYSN (SEQ ID NO: 806) |
| V_{H} Nucleotide | CAGGTGCAGCTGGTGCAGTCTGGGGGAGGCTTGGTACAGCCTGGAGGGTCCCT |
| Sequence | GAGACTCTCCTGTGCAGCCTCTTATTTCTCTTTCGCTGATTATGAAATGAGCTG |
| | GGTCCGCCAGGCTCCAGGACAACGGCTTGAGTGGGTGGCAGTTGTGTCAAAGG |
| | ATGGAAATGAACTATATTACGCAGACTCGGTGAAGGGCCGATTCACCATCTCC |
| | AGAGACAATTCCAAGAACACGCTGTATCTGCAAATGAACACCCTGAGAGCCGA |
| | GGACACAGCTGTGTATTACTGTGTGAAAGATCCTTACGATTTTTGGAGTGGTTA |
| | TTATTCGAACTGGGGCCAGGGAACCCTGGTCACCGTCTCCTCA (SEQ ID NO: |
| | 807) |
| V_{H} Amino Acid | QVQLVQSGGGLVQPGGSLRLSCAASYFSFADYEMSWVRQAPGQRLEWVAVVSK |
| Sequence | DGNELYYADSVKGRFTISRDNSKNTLYLQMNTLRAEDTAVYYCVKDPYDFWSGY |
| | YSNWGQGTLVTVSS (SEQ ID NO: 808) |

TABLE 103
Anti-CLL-1 Clone 103

| Anti-CLL-1 Clone | |
|---|---|
| 103 | Sequence |
| V_{L} CDR 1 | QASQDINNFLN (SEQ ID NO: 809) |
| V_{L} CDR 2 | DASNLET (SEQ ID NO: 157) |
| V_{L} CDR 3 | QQYGNLPFT (SEQ ID NO: 810) |
| V_{H} CDR 1 | SYYWS (SEQ ID NO: 811) |
| V_{H} CDR 2 | YIYYSGSTNYNPSLKS (SEQ ID NO: 812) |
| V_{H} CDR 3 | LVYCGGDCYSGFDY (SEQ ID NO: 813) |
| V_{L} Nucleotide | GACATCCAGCTGACCCAGAGCCCCAGCAGCCTGAGCGCCAGCGTGGGCGACAG |
| Sequence | GGTGAGCTTCACCTGCCAGGCCAGCCAGGACATCAACAACTTCCTGAACTGGT |
| | ACCAGCAGAAGCCCGGCAAGGCCCCCAAGCTGCTGATCTACGACGCCAGCAAC |
| | CTGGAGACCGGCGTGCCCAGCAGGTTCAGCGGCAGCGGCAGCGGCACCGACTT |
| | CACCTTCACCATCAGCAGCCTGCAGCCCGAGGACATCGCCACCTACTACTGCC |
| | AGCAGTACGGCAACCTGCCCTTCACCTTCGGCGGCGGCACCAAGGTGGAGATC |
| | AAGAGG (SEQ ID NO: 814) |
| V_{L} Amino Acid | DIQLTQSPSSLSASVGDRVSFTCQASQDINNFLNWYQQKPGKAPKLLIYDASNLET |
| Sequence | GVPSRFSGSGSGTDFTFTISSLQPEDIATYYCQQYGNLPFTFGGGTKVEIK (SEQ ID |
| | NO: 815) |
| Linker Amino Acid | GGGGSGGGGSGGGGS (SEQ ID NO: 361) |
| Sequence | |
| V_{H} Nucleotide | CAGGTGCAGCTGCAGGAGAGCGGCCCCGGCCTGGTGAAGCCCAGCGAGACCCT |
| Sequence | GAGCCTGACCTGCACCGTGAGCGGCGGCAGCATCAGCAGCTACTACTGGAGCT |
| | GGATCAGGCAGCCCCCCGGCAAGGGCCTGGAGTGGATCGGCTACATCTACTAC |
| | AGCGGCAGCACCAACTACAACCCCAGCCTGAAGAGCAGGGTGACCATCAGCGT |
| | GGACACCAGCAAGAACCAGTTCAGCCTGAAGCTGAGCAGCGTGACCGCCGCCG |
| | ACACCGCCGTGTACTACTGCGTGAGCCTGGTGTACTGCGGCGGCGACTGCTAC |
| | AGCGGCTTCGACTACTGGGGCCAGGGCACCCTGGTGACCGTGAGCAGC (SEQ |
| | ID NO: 816) |
| V_{H} Amino Acid | QVQLQESGPGLVKPSETLSLTCTVSGGSISSYYWSWIRQPPGKGLEWIGYIYYSGST |
| Sequence | NYNPSLKSRVTISVDTSKNQFSLKLSSVTAADTAVYYCVSLVYCGGDCYSGFDYW |
| | GQGTLVTVSS (SEQ ID NO: 817) |
| scFv Nucleotide | GACATCCAGCTGACCCAGAGCCCCAGCAGCCTGAGCGCCAGCGTGGGCGACAG |
| Sequence | GGTGAGCTTCACCTGCCAGGCCAGCCAGGACATCAACAACTTCCTGAACTGGT |
| | ACCAGCAGAAGCCCGGCAAGGCCCCCAAGCTGCTGATCTACGACGCCAGCAAC |
| | CTGGAGACCGGCGTGCCCAGCAGGTTCAGCGGCAGCGGCAGCGGCACCGACTT |
| | CACCTTCACCATCAGCAGCCTGCAGCCCGAGGACATCGCCACCTACTACTGCC |
| | AGCAGTACGGCAACCTGCCCTTCACCTTCGGCGGCGGCACCAAGGTGGAGATC |
| | AAGAGGGGCGGCGGCGGCAGCGGCGGCGGCGGCAGCGGCGGCGGCGGCAGCC |
| | AGGTGCAGCTGCAGGAGAGCGGCCCCGGCCTGGTGAAGCCCAGCGAGACCCT |
| | GAGCCTGACCTGCACCGTGAGCGGCGGCAGCATCAGCAGCTACTACTGGAGCT |
| | GGATCAGGCAGCCCCCCGGCAAGGGCCTGGAGTGGATCGGCTACATCTACTAC |
| | AGCGGCAGCACCAACTACAACCCCAGCCTGAAGAGCAGGGTGACCATCAGCGT |
| | GGACACCAGCAAGAACCAGTTCAGCCTGAAGCTGAGCAGCGTGACCGCCGCCG |
| | ACACCGCCGTGTACTACTGCGTGAGCCTGGTGTACTGCGGCGGCGACTGCTAC |
| | AGCGGCTTCGACTACTGGGGCCAGGGCACCCTGGTGACCGTGAGCAGC (SEQ |
| | ID NO: 818) |
| scFv Amino Acid | DIQLTQSPSSLSASVGDRVSFTCQASQDINNFLNWYQQKPGKAPKLLIYDASNLET |
| Sequence | GVPSRFSGSGSGTDFTFTISSLQPEDIATYYCQQYGNLPFTFGGGTKVEIKRGGGGS |
| | GGGGSGGGGSQVQLQESGPGLVKPSETLSLTCTVSGGSISSYYWSWIRQPPGKGLE |
| | WIGYIYYSGSTNYNPSLKSRVTISVDTSKNQFSLKLSSVTAADTAVYYCVSLVYCG |
| | GDCYSGFDYWGQGTLVTVSS (SEQ ID NO: 819) |

TABLE 104
Anti-CLL-1 Humanized Clone 104

| Anti-CLL-1 | |
|---|---|
| Humanized Clone | |
| 104 | Sequence |
| V_{L} CDR 1 | KSSQSLLYSDNQKNYLA (SEQ ID NO: 820) |
| V_{L} CDR 2 | WASTRES (SEQ ID NO: 821) |
| V_{L} CDR 3 | QQYYTYPYT (SEQ ID NO: 822) |
| V_{H} CDR 1 | GYHMH (SEQ ID NO: 823) |
| V_{H} CDR 2 | RINPYNGAASHNQKFKD (SEQ ID NO: 824) |
| V_{H} CDR 3 | GWDYDGGYYAMDY (SEQ ID NO: 825) |
| V_{L} Nucleotide | GATATCCAGATGACCCAGTCTCCTAGTAGCCTGTCTGCCTCCGTCGGCGATCGG |
| Sequence | GTGACCATTACTTGCAAATCCTCACAGAGCCTCCTCTACTCCGATAATCAGAAG |
| | AACTACCTCGCCTGGTATCAACAGAAACCAGGGAAGGCACCTAAGCTGCTGAT |
| | CTACTGGGCTAGTACCCGCGAATCCGGCGTCCCTAGCAGGTTCTCTGGCAGCG |
| | GGAGCGGGACAGATTTCACCCTCACTATCTCCTCCCTGCAGCCTGAAGACTTCG |
| | CAACTTACTACTGTCAGCAGTATTATACTTACCCATACACTTTCGGACAGGGAA |
| | CAAAACTGGAAATTAAA (SEQ ID NO: 826) |
| V_{L} Amino Acid | DIQMTQSPSSLSASVGDRVTITCKSSQSLLYSDNQKNYLAWYQQKPGKAPKLLIY |
| Sequence | WASTRESGVPSRFSGSGSGTDFTLTISSLQPEDFATYYCQQYYTYPYTFGQGTKLEI |
| | K (SEQ ID NO: 827) |
| Linker Amino Acid | GGGGSGGGGSGGGGS (SEQ ID NO: 361) |
| Sequence | |
| VH Nucleotide | GAAGTCCAACTGGTGCAGAGCGGTGCAGAGGTGAAGAAGCCTGGAGCATCAG |
| Sequence | TGAAGGTGTCTTGCAAAGCCAGTGGCTACACATTCACTGGATATCATATGCATT |
| | GGGTTAGGCAGGCACCCGGCCAGCGGCTGGAGTGGATGGGAAGAATCAACCC |
| | TTATAATGGCGCTGCCTCTCACAATCAAAAGTTTAAGGATCGGGTCACTATCAC |
| | TCGGGACACTTCCGCAAGCACCGCCTATATGGAGCTGAGCAGCCTGCGGAGTG |
| | AAGACACAGCAGTCTACTACTGTGCTCGCGGATGGGACTATGACGGCGGTTAT |
| | TATGCCATGGATTACTGGGGACAGGGCACACTGGTCACCGTGAGCAGC (SEQ |
| | ID NO: 828) |
| V_{H} Amino Acid | EVQLVQSGAEVKKPGASVKVSCKASGYTFTGYHMHWVRQAPGQRLEWMGRINP |
| Sequence | YNGAASHNQKFKDRVTITRDTSASTAYMELSSLRSEDTAVYYCARGWDYDGGYY |
| | AMDYWGQGTLVTVSS (SEQ ID NO: 829) |
| scFv Nucleotide | GATATCCAGATGACCCAGTCTCCTAGTAGCCTGTCTGCCTCCGTCGGCGATCGG |
| Sequence | GTGACCATTACTTGCAAATCCTCACAGAGCCTCCTCTACTCCGATAATCAGAAG |
| | AACTACCTCGCCTGGTATCAACAGAAACCAGGGAAGGCACCTAAGCTGCTGAT |
| | CTACTGGGCTAGTACCCGCGAATCCGGCGTCCCTAGCAGGTTCTCTGGCAGCG |
| | GGAGCGGGACAGATTTCACCCTCACTATCTCCTCCCTGCAGCCTGAAGACTTCG |
| | CAACTTACTACTGTCAGCAGTATTATACTTACCCATACACTTTCGGACAGGGAA |
| | CAAAACTGGAAATTAAAGGTGGAGGTGGATCTGGTGGCGGTGGCAGTGGCGG |
| | AGGCGGGTCTGAAGTCCAACTGGTGCAGAGCGGTGCAGAGGTGAAGAAGCCT |
| | GGAGCATCAGTGAAGGTGTCTTGCAAAGCCAGTGGCTACACATTCACTGGATA |
| | TCATATGCATTGGGTTAGGCAGGCACCCGGCCAGCGGCTGGAGTGGATGGGAA |
| | GAATCAACCCTTATAATGGCGCTGCCTCTCACAATCAAAAGTTTAAGGATCGG |
| | GTCACTATCACTCGGGACACTTCCGCAAGCACCGCCTATATGGAGCTGAGCAG |
| | CCTGCGGAGTGAAGACACAGCAGTCTACTACTGTGCTCGCGGATGGGACTATG |
| | ACGGCGGTTATTATGCCATGGATTACTGGGGACAGGGCACACTGGTCACCGTG |
| | AGCAGC (SEQ ID NO: 830) |
| scFv Amino Acid | DIQMTQSPSSLSASVGDRVTITCKSSQSLLYSDNQKNYLAWYQQKPGKAPKLLIY |
| Sequence | WASTRESGVPSRFSGSGSGTDFTLTISSLQPEDFATYYCQQYYTYPYTFGQGTKLEI |
| | KGGGGSGGGGSGGGGSEVQLVQSGAEVKKPGASVKVSCKASGYTFTGYHMHWV |
| | RQAPGQRLEWMGRINPYNGAASHNQKFKDRVTITRDTSASTAYMELSSLRSEDTA |
| | VYYCARGWDYDGGYYAMDYWGQGTLVTVSS (SEQ ID NO: 831) |

TABLE 105
Anti-CLL-1 Mouse Clone 104

| Anti-CLL-1 Mouse | |
|---|---|
| Clone 104 | Sequence |
| V_{L} CDR 1 | KSSQSLLYSDNQKNYLA (SEQ ID NO: 820) |
| V_{L} CDR 2 | WASTRES (SEQ ID NO: 821) |
| V_{L} CDR 3 | QQYYTYPYT (SEQ ID NO: 822) |
| V_{H} CDR 1 | GYHMH (SEQ ID NO: 823) |
| V_{H} CDR 2 | RINPYNGAASHNQKFKD (SEQ ID NO: 824) |
| V_{H} CDR 3 | GWDYDGGYYAMDY (SEQ ID NO: 825) |
| V_{L} Nucleotide | GACATCGTGATGAGCCAGAGCCCCAGCAGCCTGGCCGTGAGCGTGGGCGAGA |
| Sequence | AGGTGACCATGAGCTGCAAGAGCAGCCAGAGCCTGCTGTACAGCGACAACCA |
| | GAAGAACTACCTGGCCTGGTACCAGCAGAAGCCCGGCCAGAGCCCCAAGCTGC |
| | TGATCTACTGGGCCAGCACCAGGGAGAGCGGCGTGCCCGACAGGTTCACCGGC |
| | AGCGGCAGCGGCACCGACTTCACCCTGACCATCAGCAGCGTGAAGGCCGAGGA |
| | CCTGGCCGTGTACTACTGCCAGCAGTACTACACCTACCCCTACACCTTCGGCGG |
| | CGGCACCAAGCTGGAGATCAAG (SEQ ID NO: 832) |
| V_{L} Amino Acid | DIVMSQSPSSLAVSVGEKVTMSCKSSQSLLYSDNQKNYLAWYQQKPGQSPKLLIY |
| Sequence | WASTRESGVPDRFTGSGSGTDFTLTISSVKAEDLAVYYCQQYYTYPYTFGGGTKLE |
| | IK (SEQ ID NO: 833) |
| Linker Amino Acid | GGGGSGGGGSGGGGS (SEQ ID NO: 361) |
| Sequence | |
| V_{H} Nucleotide | GAGGTGCAGCTGCAGCAGAGCGGCCCCGAGCTGGTGAAGCCCGGCGCCAGCG |
| Sequence | TGAAGATCAGCTGCAAGGCCAGCGGCTACAGCTTCACCGGCTACCACATGCAC |
| | TGGGTGAAGCAGAGCCACGTGAAGAGCCTGGAGTGGATCGGCAGGATCAACC |
| | CCTACAACGGCGCCGCCAGCCACAACCAGAAGTTCAAGGACAAGGCCACCCTG |
| | ACCGTGGACAAGAGCAGCAGCACCGCCTACATGGAGCTGCACAGCCTGACCAG |
| | CGAGGACAGCGCCGTGTACTACTGCGCCAGGGGCTGGGACTACGACGGCGGCT |
| | ACTACGCCATGGACTACTGGGGCCAGGGCACCAGCGTGACCGTGAGCAGC |
| | (SEQ ID NO: 834) |
| V_{H} Amino Acid | EVQLQQSGPELVKPGASVKISCKASGYSFTGYHMHWVKQSHVKSLEWIGRINPYN |
| Sequence | GAASHNQKFKDKATLTVDKSSSTAYMELHSLTSEDSAVYYCARGWDYDGGYYA |
| | MDYWGQGTSVTVSS (SEQ ID NO: 835) |
| scFv Nucleotide | GACATCGTGATGAGCCAGAGCCCCAGCAGCCTGGCCGTGAGCGTGGGCGAGA |
| Sequence | AGGTGACCATGAGCTGCAAGAGCAGCCAGAGCCTGCTGTACAGCGACAACCA |
| | GAAGAACTACCTGGCCTGGTACCAGCAGAAGCCCGGCCAGAGCCCCAAGCTGC |
| | TGATCTACTGGGCCAGCACCAGGGAGAGCGGCGTGCCCGACAGGTTCACCGGC |
| | AGCGGCAGCGGCACCGACTTCACCCTGACCATCAGCAGCGTGAAGGCCGAGGA |
| | CCTGGCCGTGTACTACTGCCAGCAGTACTACACCTACCCCTACACCTTCGGCGG |
| | CGGCACCAAGCTGGAGATCAAGGGCGGCGGCGGCAGCGGCGGCGGCGGCAGC |
| | GGCGGCGGCGGCAGCGAGGTGCAGCTGCAGCAGAGCGGCCCCGAGCTGGTGA |
| | AGCCCGGCGCCAGCGTGAAGATCAGCTGCAAGGCCAGCGGCTACAGCTTCACC |
| | GGCTACCACATGCACTGGGTGAAGCAGAGCCACGTGAAGAGCCTGGAGTGGAT |
| | CGGCAGGATCAACCCCTACAACGGCGCCGCCAGCCACAACCAGAAGTTCAAGG |
| | ACAAGGCCACCCTGACCGTGGACAAGAGCAGCAGCACCGCCTACATGGAGCTG |
| | CACAGCCTGACCAGCGAGGACAGCGCCGTGTACTACTGCGCCAGGGGCTGGGA |
| | CTACGACGGCGGCTACTACGCCATGGACTACTGGGGCCAGGGCACCAGCGTGA |
| | CCGTGAGCAGC (SEQ ID NO: 836) |
| scFv Amino Acid | DIVMSQSPSSLAVSVGEKVTMSCKSSQSLLYSDNQKNYLAWYQQKPGQSPKLLIY |
| Sequence | WASTRESGVPDRFTGSGSGTDFTLTISSVKAEDLAVYYCQQYYTYPYTFGGGTKLE |
| | IKGGGGSGGGGSGGGGSEVQLQQSGPELVKPGASVKISCKASGYSFTGYHMHWV |
| | KQSHVKSLEWIGRINPYNGAASHNQKFKDKATLTVDKSSSTAYMELHSLTSEDSA |
| | VYYCARGWDYDGGYYAMDYWGQGTSVTVSS (SEQ ID NO: 837) |

TABLE 106
Anti-CLL-1 Binder Clone 106

| Anti-CLL-1 Binder | |
|---|---|
| Clone 106 | Sequence |
| V_{L} CDR 1 | SASSSVSYMY (SEQ ID NO: 838) |
| V_{L} CDR 2 | DTSNLAS (SEQ ID NO: 839) |
| V_{L} CDR 3 | QQWSSYPLT (SEQ ID NO: 840) |
| V_{H} CDR 1 | SYWIE (SEQ ID NO: 841) |
| V_{H} CDR 2 | EIFPGSGSIKYNEKFKG (SEQ ID NO: 842) |
| V_{H} CDR 3 | GGTYNDYSLFDY (SEQ ID NO: 843) |
| V_{L} Nucleotide | CAGATCGTGCTGACCCAGAGCCCCGCCATCATGAGCGCCAGCCCCGGCGAGAA |
| Sequence | GGTGACCATGACCTGCAGCGCCAGCAGCAGCGTGAGCTACATGTACTGGTACC |
| | AGCAGAAGCCCGGCAGCAGCCCCAGGCTGCTGATCTTCGACACCAGCAACCTG |
| | GCCAGCGGCGTGCCCGTGAGGTTCAGCGGCAGCGGCAGCGGCACCAGCTACAG |
| | CCTGACCATCAGCAGGATGGAGGCCGAGGACGCCGCCACCTACTACTGCCAGC |
| | AGTGGAGCAGCTACCCCCTGACCTTCGGCGCCGGCACCAAGCTGGAGCTGAAG |
| | (SEQ ID NO: 844) |
| V_{L} Amino Acid | QIVLTQSPAIMSASPGEKVTMTCSASSSVSYMYWYQQKPGSSPRLLIFDTSNLASG |
| Sequence | VPVRFSGSGSGTSYSLTISRMEAEDAATYYCQQWSSYPLTFGAGTKLELK (SEQ ID |
| | NO: 845) |
| Linker Amino Acid | GGGGSGGGGSGGGGS (SEQ ID NO: 361) |
| Sequence | |
| V_{H} Nucleotide | CAGGTGCAGCTGCAGCAGAGCGGCGCCGAGCTGATGAAGCCCGGCGCCAGCG |
| Sequence | TGAAGATCAGCTGCAAGGCCACCGGCTACACCTTCAGCAGCTACTGGATCGAG |
| | TGGGTGAAGCAGAGGCCCGGCCACGGCCTGGAGTGGATCGGCGAGATCTTCCC |
| | CGGCAGCGGCAGCATCAAGTACAACGAGAAGTTCAAGGGCAAGGCCACCTTC |
| | ACCGCCGACACCAGCAGCAACACCGCCTACATGCAGCTGAGCAGCCTGACCAG |
| | CGAGGACAGCGCCGTGCACTACTGCGCCAGGGGCGGCACCTACAACGACTACA |
| | GCCTGTTCGACTACTGGGGCCAGGGCACCACCCTGACCGTGAGCAGC (SEQ ID |
| | NO: 846) |
| V_{H} Amino Acid | QVQLQQSGAELMKPGASVKISCKATGYTFSSYWIEWVKQRPGHGLEWIGEIFPGS |
| Sequence | GSIKYNEKFKGKATFTADTSSNTAYMQLSSLTSEDSAVHYCARGGTYNDYSLFDY |
| | WGQGTTLTVSS (SEQ ID NO: 847) |
| scFv Nucleotide | CAGATCGTGCTGACCCAGAGCCCCGCCATCATGAGCGCCAGCCCCGGCGAGAA |
| Sequence | GGTGACCATGACCTGCAGCGCCAGCAGCAGCGTGAGCTACATGTACTGGTACC |
| | AGCAGAAGCCCGGCAGCAGCCCCAGGCTGCTGATCTTCGACACCAGCAACCTG |
| | GCCAGCGGCGTGCCCGTGAGGTTCAGCGGCAGCGGCAGCGGCACCAGCTACAG |
| | CCTGACCATCAGCAGGATGGAGGCCGAGGACGCCGCCACCTACTACTGCCAGC |
| | AGTGGAGCAGCTACCCCCTGACCTTCGGCGCCGGCACCAAGCTGGAGCTGAAG |
| | GGCGGCGGCGGCAGCGGCGGCGGCGGCAGCGGCGGCGGCGGCAGCCAGGTGC |
| | AGCTGCAGCAGAGCGGCGCCGAGCTGATGAAGCCCGGCGCCAGCGTGAAGAT |
| | CAGCTGCAAGGCCACCGGCTACACCTTCAGCAGCTACTGGATCGAGTGGGTGA |
| | AGCAGAGGCCCGGCCACGGCCTGGAGTGGATCGGCGAGATCTTCCCCGGCAGC |
| | GGCAGCATCAAGTACAACGAGAAGTTCAAGGGCAAGGCCACCTTCACCGCCGA |
| | CACCAGCAGCAACACCGCCTACATGCAGCTGAGCAGCCTGACCAGCGAGGACA |
| | GCGCCGTGCACTACTGCGCCAGGGGCGGCACCTACAACGACTACAGCCTGTTC |
| | GACTACTGGGGCCAGGGCACCACCCTGACCGTGAGCAGC (SEQ ID NO: 848) |
| scFv Amino Acid | QIVLTQSPAIMSASPGEKVTMTCSASSSVSYMYWYQQKPGSSPRLLIFDTSNLASG |
| Sequence | VPVRFSGSGSGTSYSLTISRMEAEDAATYYCQQWSSYPLTFGAGTKLELKGGGGS |
| | GGGGSGGGGSQVQLQQSGAELMKPGASVKISCKATGYTFSSYWIEWVKQRPGHG |
| | LEWIGEIFPGSGSIKYNEKFKGKATFTADTSSNTAYMQLSSLTSEDSAVHYCARGG |
| | TYNDYSLFDYWGQGTTLTVSS (SEQ ID NO: 849) |

TABLE 107
Anti-CLL-1 Human Clone 107

| Anti-CLL-1 Human | |
|---|---|
| Clone 107 | Sequence |
| V_{L} CDR 1 | SASSSVSYIY (SEQ ID NO: 850) |
| V_{L} CDR 2 | DTSNLAS (SEQ ID NO: 839) |
| V_{L} CDR 3 | QQWSSFPPT (SEQ ID NO: 851) |
| V_{H} CDR 1 | RYWMH (SEQ ID NO: 852) |
| V_{H} CDR 2 | YIYPGSGTSNYDEKFKS (SEQ ID NO: 853) |
| V_{H} CDR 3 | EARYTMDY (SEQ ID NO: 854) |
| V_{L} Nucleotide | CAGATCGTGCTGACCCAGAGCCCCGCCATCATGAGCGCCAGCCCCGGCGAGAA |
| Sequence | GGTGACCATGACCTGCAGCGCCAGCAGCAGCGTGAGCTACATCTACTGGTACC |
| | AGCAGAAGCCCGGCAGCAGCCCCGGCCTGCTGATCTACGACACCAGCAACCTG |
| | GCCAGCGGCGTGCCCGTGAGGTTCAGCGGCAGCGGCAGCGGCACCAGCTACAG |
| | CCTGACCATCAGCAGGATGGAGGCCGAGGACGCCGCCACCTACTACTGCCAGC |
| | AGTGGAGCAGCTTCCCCCCCACCTTCGGCGCCGGCACCAAGCTGGAGCTGAAG |
| | (SEQ ID NO: 855) |
| V_{L} Amino Acid | QIVLTQSPAIMSASPGEKVTMTCSASSSVSYIYWYQQKPGSSPGLLIYDTSNLASGV |
| Sequence | PVRFSGSGSGTSYSLTISRMEAEDAATYYCQQWSSFPPTFGAGTKLELK (SEQ ID |
| | NO: 856) |
| Linker Amino Acid | GGGGSGGGGSGGGGS (SEQ ID NO: 361) |
| Sequence | |
| V_{H} Nucleotide | CAGGTGCAGCTGCAGCAGCCCGGCAGCGACCTGGTGAGGCCCGGCGCCAGCGT |
| Sequence | GAAGCTGAGCTGCAAGGCCAGCGGCTACACCTTCACCAGGTACTGGATGCACT |
| | GGGTGAAGCAGAGGCCCGGCCACGGCCTGGAGTGGATCGGCTACATCTACCCC |
| | GGCAGCGGCACCAGCAACTACGACGAGAAGTTCAAGAGCAAGGCCACCCTGA |
| | CCGTGGACACCAGCAGCAGCACCGCCTACATGCAGCTGAGCAGCCTGACCAGC |
| | GAGGACAGCGCCGTGTACTACTGCACCAGGGAGGCCAGGTACACCATGGACTA |
| | CTGGGGCCAGGGCACCAGCGTGACCGTGAGCAGC (SEQ ID NO: 857) |
| V_{H} Amino Acid | QVQLQQPGSDLVRPGASVKLSCKASGYTFTRYWMHWVKQRPGHGLEWIGYIYPG |
| Sequence | SGTSNYDEKFKSKATLTVDTSSSTAYMQLSSLTSEDSAVYYCTREARYTMDYWGQ |
| | GTSVTVSS (SEQ ID NO: 858) |
| scFv Nucleotide | CAGATCGTGCTGACCCAGAGCCCCGCCATCATGAGCGCCAGCCCCGGCGAGAA |
| Sequence | GGTGACCATGACCTGCAGCGCCAGCAGCAGCGTGAGCTACATCTACTGGTACC |
| | AGCAGAAGCCCGGCAGCAGCCCCGGCCTGCTGATCTACGACACCAGCAACCTG |
| | GCCAGCGGCGTGCCCGTGAGGTTCAGCGGCAGCGGCAGCGGCACCAGCTACAG |
| | CCTGACCATCAGCAGGATGGAGGCCGAGGACGCCGCCACCTACTACTGCCAGC |
| | AGTGGAGCAGCTTCCCCCCCACCTTCGGCGCCGGCACCAAGCTGGAGCTGAAG |
| | GGCGGCGGCGGCAGCGGCGGCGGCGGCAGCGGCGGCGGCGGCAGCCAGGTGC |
| | AGCTGCAGCAGCCCGGCAGCGACCTGGTGAGGCCCGGCGCCAGCGTGAAGCTG |
| | AGCTGCAAGGCCAGCGGCTACACCTTCACCAGGTACTGGATGCACTGGGTGAA |
| | GCAGAGGCCCGGCCACGGCCTGGAGTGGATCGGCTACATCTACCCCGGCAGCG |
| | GCACCAGCAACTACGACGAGAAGTTCAAGAGCAAGGCCACCCTGACCGTGGA |
| | CACCAGCAGCAGCACCGCCTACATGCAGCTGAGCAGCCTGACCAGCGAGGACA |
| | GCGCCGTGTACTACTGCACCAGGGAGGCCAGGTACACCATGGACTACTGGGGC |
| | CAGGGCACCAGCGTGACCGTGAGCAGC (SEQ ID NO: 859) |
| scFv Amino Acid | QIVLTQSPAIMSASPGEKVTMTCSASSSVSYIYWYQQKPGSSPGLLIYDTSNLASGV |
| Sequence | PVRFSGSGSGTSYSLTISRMEAEDAATYYCQQWSSFPPTFGAGTKLELKGGGGSGG |
| | GGSGGGGSQVQLQQPGSDLVRPGASVKLSCKASGYTFTRYWMHWVKQRPGHGL |
| | EWIGYIYPGSGTSNYDEKFKSKATLTVDTSSSTAYMQLSSLTSEDSAVYYCTREAR |
| | YTMDYWGQGTSVTVSS (SEQ ID NO: 860) |

TABLE 108
Anti-CLL-1 Clone 108

| Anti-CLL-1 Clone | |
|---|---|
| 108 | Sequence |
| V_{L} CDR 1 | QASQDVRTWLA (SEQ ID NO: 861) |
| V_{L} CDR 2 | SASSLQT (SEQ ID NO: 862) |
| V_{L} CDR 3 | QQSNYLPFT (SEQ ID NO: 863) |
| V_{H} CDR 1 | SYDMS (SEQ ID NO: 864) |
| V_{H} CDR 2 | AINPSGDFTSYNEKFQG (SEQ ID NO: 865) |
| V_{H} CDR 3 | GVGWSFLDY (SEQ ID NO: 866) |
| V_{L} Nucleotide | GACATCCAGATGACCCAGAGCCCCAGCAGCCTGAGCGCCAGCGTGGGCGACA |
| Sequence | GGGTGACCATCACCTGCCAGGCCAGCCAGGACGTGAGGACCTGGCTGGCCTGG |
| | TACCAGCAGAAGCCCGGCAAGGCCCCCAAGCTGCTGATCTACAGCGCCAGCAG |
| | CCTGCAGACCGGCGTGCCCAGCAGGTTCAGCGGCAGCGGCAGCGGCACCGACT |
| | TCACCCTGACCATCAGCAGCCTGCAGCCCGAGGACTTCGCCACCTACTACTGCC |
| | AGCAGAGCAACTACCTGCCCTTCACCTTCGGCCAGGGCACCAAGGTGGAGATC |
| | AAGAGCAGC (SEQ ID NO: 867) |
| V_{L} Amino Acid | DIQMTQSPSSLSASVGDRVTITCQASQDVRTWLAWYQQKPGKAPKLLIYSASSLQT |
| Sequence | GVPSRFSGSGSGTDFTLTISSLQPEDFATYYCQQSNYLPFTFGQGTKVEIKSS (SEQ |
| | ID NO: 868) |
| Linker Amino Acid | GGGGSGGGGSGGGGS (SEQ ID NO: 361) |
| Sequence | |
| V_{H} Nucleotide | CAGGTGCAGCTGGTGCAGAGCGGCGCCGAGGTGAAGAAGCCCGGCGCCAGCG |
| Sequence | TGAAGGTGAGCTGCAAGGCCAGCGGCTACACCTTCACCAGCTACGACATGAGC |
| | TGGGTGAGGCAGGCCCCCGGCCAGGGCCTGGAGTGGATGGGCGCCATCAACCC |
| | CAGCGGCGACTTCACCAGCTACAACGAGAAGTTCCAGGGCAGGGTGACCATGA |
| | CCAGGGACACCAGCACCAGCACCGTGTACATGGAGCTGAGCAGCCTGAGGAG |
| | CGAGGACACCGCCGTGTACTACTGCGCCAGGGGCGTGGGCTGGAGCTTCCTGG |
| | ACTACTGGGGCCAGGGCACCCTGGTGACCGTGAGCAGC (SEQ ID NO: 869) |
| V_{H} Amino Acid | QVQLVQSGAEVKKPGASVKVSCKASGYTFTSYDMSWVRQAPGQGLEWMGAINP |
| Sequence | SGDFTSYNEKFQGRVTMTRDTSTSTVYMELSSLRSEDTAVYYCARGVGWSFLDY |
| | WGQGTLVTVSS (SEQ ID NO: 870) |
| scFv Nucleotide | CAGGTGCAGCTGGTGCAGAGCGGCGCCGAGGTGAAGAAGCCCGGCGCCAGCG |
| Sequence | TGAAGGTGAGCTGCAAGGCCAGCGGCTACACCTTCACCAGCTACGACATGAGC |
| | TGGGTGAGGCAGGCCCCCGGCCAGGGCCTGGAGTGGATGGGCGCCATCAACCC |
| | CAGCGGCGACTTCACCAGCTACAACGAGAAGTTCCAGGGCAGGGTGACCATGA |
| | CCAGGGACACCAGCACCAGCACCGTGTACATGGAGCTGAGCAGCCTGAGGAG |
| | CGAGGACACCGCCGTGTACTACTGCGCCAGGGGCGTGGGCTGGAGCTTCCTGG |
| | ACTACTGGGGCCAGGGCACCCTGGTGACCGTGAGCAGCGGCGGCGGCGGCAG |
| | CGGCGGCGGCGGCAGCGGCGGCGGCGGCAGCGACATCCAGATGACCCAGAGC |
| | CCCAGCAGCCTGAGCGCCAGCGTGGGCGACAGGGTGACCATCACCTGCCAGGC |
| | CAGCCAGGACGTGAGGACCTGGCTGGCCTGGTACCAGCAGAAGCCCGGCAAG |
| | GCCCCCAAGCTGCTGATCTACAGCGCCAGCAGCCTGCAGACCGGCGTGCCCAG |
| | CAGGTTCAGCGGCAGCGGCAGCGGCACCGACTTCACCCTGACCATCAGCAGCC |
| | TGCAGCCCGAGGACTTCGCCACCTACTACTGCCAGCAGAGCAACTACCTGCCC |
| | TTCACCTTCGGCCAGGGCACCAAGGTGGAGATCAAGAGCAGC (SEQ ID NO: |
| | 871) |
| scFv Amino Acid | QVQLVQSGAEVKKPGASVKVSCKASGYTFTSYDMSWVRQAPGQGLEWMGAINP |
| Sequence | SGDFTSYNEKFQGRVTMTRDTSTSTVYMELSSLRSEDTAVYYCARGVGWSFLDY |
| | WGQGTLVTVSSGGGGSGGGGSGGGGSDIQMTQSPSSLSASVGDRVTITCQASQDV |
| | RTWLAWYQQKPGKAPKLLIYSASSLQTGVPSRFSGSGSGTDFTLTISSLQPEDFATY |
| | YCQQSNYLPFTFGQGTKVEIKSS (SEQ ID NO: 872) |

TABLE 109
Anti-CLL-1 Clone 109

| Anti-CLL-1 Clone | |
|---|---|
| 109 | Sequence |
| V_{L} CDR 1 | KASEDIHSDLA (SEQ ID NO: 873) |
| V_{L} CDR 2 | SASSLQS (SEQ ID NO: 129) |
| V_{L} CDR 3 | QQAYYLPLT (SEQ ID NO: 874) |
| V_{H} CDR 1 | DYAMD (SEQ ID NO: 875) |
| V_{H} CDR 2 | WISGQSGSTSYNEKFQG (SEQ ID NO: 876) |
| V_{H} CDR 3 | WLDGPWFDV (SEQ ID NO: 877) |
| V_{L} Nucleotide | GACATCCAGATGACCCAGAGCCCCAGCAGCCTGAGCGCCAGCGTGGGCGACA |
| Sequence | GGGTGACCATCACCTGCAAGGCCAGCGAGGACATCCACAGCGACCTGGCCTGG |
| | TACCAGCAGAAGCCCGGCAAGGCCCCCAAGCTGCTGATCTACAGCGCCAGCAG |
| | CCTGCAGAGCGGCGTGCCCAGCAGGTTCAGCGGCAGCGGCAGCGGCACCGACT |
| | TCACCCTGACCATCAGCAGCCTGCAGCCCGAGGACTTCGCCACCTACTACTGCC |
| | AGCAGGCCTACTACCTGCCCCTGACCTTCGGCCAGGGCACCAAGGTGGAGATC |
| | AAGAGCAGC (SEQ ID NO: 878) |
| V_{L} Amino Acid | DIQMTQSPSSLSASVGDRVTITCKASEDIHSDLAWYQQKPGKAPKLLIYSASSLQSG |
| Sequence | VPSRFSGSGSGTDFTLTISSLQPEDFATYYCQQAYYLPLTFGQGTKVEIKSS (SEQ ID |
| | NO: 879) |
| Linker Amino Acid | GGGGSGGGGSGGGGS (SEQ ID NO: 361) |
| Sequence | |
| V_{H} Nucleotide | CAGGTGCAGCTGGTGCAGAGCGGCGCCGAGGTGAAGAAGCCCGGCGCCAGCG |
| Sequence | TGAAGGTGAGCTGCAAGGCCAGCGGCTACACCTTCACCGACTACGCCATGGAC |
| | TGGGTGAGGCAGGCCCCCGGCCAGGGCCTGGAGTGGATGGGCTGGATCAGCG |
| | GCCAGAGCGGCAGCACCAGCTACAACGAGAAGTTCCAGGGCAGGGTGACCAT |
| | GACCAGGGACACCAGCACCAGCACCGTGTACATGGAGCTGAGCAGCCTGAGG |
| | AGCGAGGACACCGCCGTGTACTACTGCGCCAAGTGGCTGGACGGCCCCTGGTT |
| | CGACGTGTGGGGCCAGGGCACCCTGGTGACCGTGAGCAGC (SEQ ID NO: 880) |
| V_{H} Amino Acid | QVQLVQSGAEVKKPGASVKVSCKASGYTFTDYAMDWVRQAPGQGLEWMGWIS |
| Sequence | GQSGSTSYNEKFQGRVTMTRDTSTSTVYMELSSLRSEDTAVYYCAKWLDGPWFD |
| | VWGQGTLVTVSS (SEQ ID NO: 881) |
| scFv Nucleotide | CAGGTGCAGCTGGTGCAGAGCGGCGCCGAGGTGAAGAAGCCCGGCGCCAGCG |
| Sequence | TGAAGGTGAGCTGCAAGGCCAGCGGCTACACCTTCACCGACTACGCCATGGAC |
| | TGGGTGAGGCAGGCCCCCGGCCAGGGCCTGGAGTGGATGGGCTGGATCAGCG |
| | GCCAGAGCGGCAGCACCAGCTACAACGAGAAGTTCCAGGGCAGGGTGACCAT |
| | GACCAGGGACACCAGCACCAGCACCGTGTACATGGAGCTGAGCAGCCTGAGG |
| | AGCGAGGACACCGCCGTGTACTACTGCGCCAAGTGGCTGGACGGCCCCTGGTT |
| | CGACGTGTGGGGCCAGGGCACCCTGGTGACCGTGAGCAGCGGCGGCGGCGGC |
| | AGCGGCGGCGGCGGCAGCGGCGGCGGCGGCAGCGACATCCAGATGACCCAGA |
| | GCCCCAGCAGCCTGAGCGCCAGCGTGGGCGACAGGGTGACCATCACCTGCAAG |
| | GCCAGCGAGGACATCCACAGCGACCTGGCCTGGTACCAGCAGAAGCCCGGCA |
| | AGGCCCCCAAGCTGCTGATCTACAGCGCCAGCAGCCTGCAGAGCGGCGTGCCC |
| | AGCAGGTTCAGCGGCAGCGGCAGCGGCACCGACTTCACCCTGACCATCAGCAG |
| | CCTGCAGCCCGAGGACTTCGCCACCTACTACTGCCAGCAGGCCTACTACCTGCC |
| | CCTGACCTTCGGCCAGGGCACCAAGGTGGAGATCAAGAGCAGC (SEQ ID NO: |
| | 882) |
| scFv Amino Acid | QVQLVQSGAEVKKPGASVKVSCKASGYTFTDYAMDWVRQAPGQGLEWMGWIS |
| Sequence | GQSGSTSYNEKFQGRVTMTRDTSTSTVYMELSSLRSEDTAVYYCAKWLDGPWFD |
| | VWGQGTLVTVSSGGGGSGGGGSGGGGSDIQMTQSPSSLSASVGDRVTITCKASEDI |
| | HSDLAWYQQKPGKAPKLLIYSASSLQSGVPSRFSGSGSGTDFTLTISSLQPEDFATY |
| | YCQQAYYLPLTFGQGTKVEIKSS (SEQ ID NO: 883) |

TABLE 110
Anti-CLL-1 Clone 110

| Anti-CLL-1 Clone | |
|---|---|
| 110 | Sequence |
| V_{L} CDR 1 | QASQDVSTDLA (SEQ ID NO: 884) |
| V_{L} CDR 2 | HASSLES (SEQ ID NO: 885) |
| V_{L} CDR 3 | QQYYNYPLT (SEQ ID NO: 886) |
| V_{H} CDR 1 | HYAMD (SEQ ID NO: 887) |
| V_{H} CDR 2 | GISPQSGGTSYNEKFQG (SEQ ID NO: 888) |
| V_{H} CDR 3 | VHGDYHFDY (SEQ ID NO: 889) |
| V_{L} Nucleotide | GACATCCAGATGACCCAGAGCCCCAGCAGCCTGAGCGCCAGCGTGGGCGACA |
| Sequence | GGGTGACCATCACCTGCCAGGCCAGCCAGGACGTGAGCACCGACCTGGCCTGG |
| | TACCAGCAGAAGCCCGGCAAGGCCCCCAAGCTGCTGATCTACCACGCCAGCAG |
| | CCTGGAGAGCGGCGTGCCCAGCAGGTTCAGCGGCAGCGGCAGCGGCACCGACT |
| | TCACCCTGACCATCAGCAGCCTGCAGCCCGAGGACTTCGCCACCTACTACTGCC |
| | AGCAGTACTACAACTACCCCCTGACCTTCGGCCAGGGCACCAAGGTGGAGATC |
| | AAGAGCAGC (SEQ ID NO: 890) |
| V_{L} Amino Acid | DIQMTQSPSSLSASVGDRVTITCQASQDVSTDLAWYQQKPGKAPKLLIYHASSLES |
| Sequence | GVPSRFSGSGSGTDFTLTISSLQPEDFATYYCQQYYNYPLTFGQGTKVEIKSS (SEQ |
| | ID NO: 891) |
| Linker Amino Acid | GGGGSGGGGSGGGGS (SEQ ID NO: 361) |
| Sequence | |
| V_{H} Nucleotide | CAGGTGCAGCTGGTGCAGAGCGGCGCCGAGGTGAAGAAGCCCGGCGCCAGCG |
| Sequence | TGAAGGTGAGCTGCAAGGCCAGCGGCTACACCTTCACCCACTACGCCATGGAC |
| | TGGGTGAGGCAGGCCCCCGGCCAGGGCCTGGAGTGGATGGGCGGCATCAGCCC |
| | CCAGAGCGGCGGCACCAGCTACAACGAGAAGTTCCAGGGCAGGGTGACCATG |
| | ACCAGGGACACCAGCACCAGCACCGTGTACATGGAGCTGAGCAGCCTGAGGA |
| | GCGAGGACACCGCCGTGTACTACTGCGCCAGGGTGCACGGCGACTACCACTTC |
| | GACTACTGGGGCCAGGGCACCCTGGTGACCGTGAGCAGC (SEQ ID NO: 892) |
| V_{H} Amino Acid | QVQLVQSGAEVKKPGASVKVSCKASGYTFTHYAMDWVRQAPGQGLEWMGGISP |
| Sequence | QSGGTSYNEKFQGRVTMTRDTSTSTVYMELSSLRSEDTAVYYCARVHGDYHFDY |
| | WGQGTLVTVSS (SEQ ID NO: 893) |
| scFv Nucleotide | CAGGTGCAGCTGGTGCAGAGCGGCGCCGAGGTGAAGAAGCCCGGCGCCAGCG |
| Sequence | TGAAGGTGAGCTGCAAGGCCAGCGGCTACACCTTCACCCACTACGCCATGGAC |
| | TGGGTGAGGCAGGCCCCCGGCCAGGGCCTGGAGTGGATGGGCGGCATCAGCCC |
| | CCAGAGCGGCGGCACCAGCTACAACGAGAAGTTCCAGGGCAGGGTGACCATG |
| | ACCAGGGACACCAGCACCAGCACCGTGTACATGGAGCTGAGCAGCCTGAGGA |
| | GCGAGGACACCGCCGTGTACTACTGCGCCAGGGTGCACGGCGACTACCACTTC |
| | GACTACTGGGGCCAGGGCACCCTGGTGACCGTGAGCAGCGGCGGCGGCGGCA |
| | GCGGCGGCGGCGGCAGCGGCGGCGGCGGCAGCGACATCCAGATGACCCAGAG |
| | CCCCAGCAGCCTGAGCGCCAGCGTGGGCGACAGGGTGACCATCACCTGCCAGG |
| | CCAGCCAGGACGTGAGCACCGACCTGGCCTGGTACCAGCAGAAGCCCGGCAA |
| | GGCCCCCAAGCTGCTGATCTACCACGCCAGCAGCCTGGAGAGCGGCGTGCCCA |
| | GCAGGTTCAGCGGCAGCGGCAGCGGCACCGACTTCACCCTGACCATCAGCAGC |
| | CTGCAGCCCGAGGACTTCGCCACCTACTACTGCCAGCAGTACTACAACTACCCC |
| | CTGACCTTCGGCCAGGGCACCAAGGTGGAGATCAAGAGCAGC (SEQ ID NO: |
| | 894) |
| scFv Amino Acid | QVQLVQSGAEVKKPGASVKVSCKASGYTFTHYAMDWVRQAPGQGLEWMGGISP |
| Sequence | QSGGTSYNEKFQGRVTMTRDTSTSTVYMELSSLRSEDTAVYYCARVHGDYHFDY |
| | WGQGTLVTVSSGGGGSGGGGSGGGGSDIQMTQSPSSLSASVGDRVTITCQASQDV |
| | STDLAWYQQKPGKAPKLLIYHASSLESGVPSRFSGSGSGTDFTLTISSLQPEDFATY |
| | YCQQYYNYPLTFGQGTKVEIKSS (SEQ ID NO: 895) |

TABLE 111
Anti-CLL-1 Clone 111

| Anti-CLL-1 Binder | |
|---|---|
| Clone 111 | Sequence |
| V_{H} CDR 1 | AYFMG (SEQ ID NO: 896) |
| V_{H} CDR 2 | AINWNGDSSWYRDSVKG (SEQ ID NO: 897) |
| V_{H} CDR 3 | DTHGAVGLGSERLYDY (SEQ ID NO: 898) |
| V_{H} Nucleotide | CAGGTGCAGCTGCAGGAGAGCGGCGGCGGCCTGGTGCAGACCGGCGACAGCC |
| Sequence | TGAGGCTGAGCTGCGTGGCCAGCGGCAGGACCTTCAGCGCCTACTTCATGGGC |
| | TGGTTCAGGCAGGCCCCCGGCAAGGAGAGGGAGTTCGTGAGCGCCATCAACTG |
| | GAACGGCGACAGCAGCTGGTACAGGGACAGCGTGAAGGGCAGGTTCACCGTG |
| | AGCAGGGACAACGCCAAGAACACCGTGTACCTGCAGATGAACAGCCTGGAGC |
| | CCGAGGACACCGCCGTGTACTACTGCGCCGCCGACACCCACGGCGCCGTGGGC |
| | CTGGGCAGCGAGAGGCTGTACGACTACTGGGGCCAGGGCACCCAGGTGACCGT |
| | GAGCAGC (SEQ ID NO: 899) |
| V_{H} Amino Acid | QVQLQESGGGLVQTGDSLRLSCVASGRTFSAYFMGWFRQAPGKEREFVSAINWN |
| Sequence | GDSSWYRDSVKGRFTVSRDNAKNTVYLQMNSLEPEDTAVYYCAADTHGAVGLG |
| | SERLYDYWGQGTQVTVSS (SEQ ID NO: 900) |

TABLE 112
Anti-CLL-1 Clone 112

| Anti-CLL-1 Alpaca | |
|---|---|
| Clone 112 | Sequence |
| V_{H} CDR 1 | HMNPVT (SEQ ID NO: 901) |
| V_{H} CDR 2 | TIDSNDIPDYTDAVKG (SEQ ID NO: 902) |
| V_{H} CDR 3 | RIIGRYY (SEQ ID NO: 903) |
| V_{H} Nucleotide | GAGGTGCAGGTGCAGGAGAGCGGCGGCGGCCTGGTGCAGGCCGGCGGCAGCC |
| Sequence | TGACCCTGACCTGCACCACCACCGGCCCCGGCGTGCTGCACATGAACCCCGTG |
| | ACCAGGTACAGGCAGTTCCCCGGCCAGCAGAGGGAGATGGTGGCCACCATCGA |
| | CAGCAACGACATCCCCGACTACACCGACGCCGTGAAGGGCAGGTTCACCCTGA |
| | GCAGGGACGCCAGGAGGACCACCGCCTACCTGAGGATGAACAACCTGCAGAG |
| | CGACGACACCGCCATCCACTTCTGCAACGCCAGGATCATCGGCAGGTACTACT |
| | GGGGCCAGGGCACCCAGGTGACCGTGAGCAGC (SEQ ID NO: 904) |
| V_{H} Amino Acid | EVQVQESGGGLVQAGGSLTLTCTTTGPGVLHMNPVTRYRQFPGQQREMVATIDSN |
| Sequence | DIPDYTDAVKGRFTLSRDARRTTAYLRMNNLQSDDTAIHFCNARIIGRYYWGQGT |
| | QVTVSS (SEQ ID NO: 905) |

TABLE 113
Anti-CLL-1 Clone 113

| Anti-CLL-1 Clone | |
|---|---|
| 113 | Sequence |
| V_{H} CDR 1 | VARMA (SEQ ID NO: 906) |
| V_{H} CDR 2 | SITSFGDTNYADSVKG (SEQ ID NO: 907) |
| V_{H} CDR 3 | DNNRAFTDY (SEQ ID NO: 908) |
| V_{H} Nucleotide | GCCGTGCAGCTGGTGGACAGCGGCGGCGGCCTGGTGCAGCCCGGCGGCAGCCT |
| Sequence | GAGGCTGAGCTGCGCCGCCAGCGGCATCATCTTCAGCGTGGCCAGGATGGCCT |
| | GGTACAGGCAGGCCCCCGGCAAGCAGAGGGAGATGGTGGCCAGCATCACCAG |
| | CTTCGGCGACACCAACTACGCCGACAGCGTGAAGGGCAGGTTCACCATCAGCA |
| | GGAACAACGACCAGAACACCGTGTTCCTGCAGATGGACAGCCTGAAGCCCGAG |
| | GACGCCGCCCACTACTACTGCCACGTGGACAACAACAGGGCCTTCACCGACTA |
| | CTGGGGCCAGGGCACCCAGGTGACCGTGAGCAGC (SEQ ID NO: 909) |
| V_{H} Amino Acid | AVQLVDSGGGLVQPGGSLRLSCAASGIIFSVARMAWYRQAPGKQREMVASITSFG |
| Sequence | DTNYADSVKGRFTISRNNDQNTVFLQMDSLKPEDAAHYYCHVDNNRAFTDYWG |
| | QGTQVTVSS (SEQ ID NO: 910) |

TABLE 114
Anti-CLL-1 Naive Clone 114

| Anti-CLL-1 Human | |
|---|---|
| Naive Clone 114 | Sequence |
| V_{L} CDR 1 | RASQSVGNYLA (SEQ ID NO: 911) |
| V_{L} CDR 2 | DASTRAT (SEQ ID NO: 912) |
| V_{L} CDR 3 | QQSYSYPFT (SEQ ID NO: 913) |
| V_{H} CDR 1 | SYAMN (SEQ ID NO: 914) |
| V_{H} CDR 2 | WINPGSGNTNYADSVKG (SEQ ID NO: 915) |
| V_{H} CDR 3 | YAWDLDAFDY (SEQ ID NO: 916) |
| V_{L} Nucleotide | GACATCCAGATGACCCAGAGCCCCAGCAGCCTGAGCGCCAGCGTGGGCGACA |
| Sequence | GGGTGACCATCACCTGCAGGGCCAGCCAGAGCGTGGGCAACTACCTGGCCTGG |
| | TACCAGCAGAAGCCCGGCAAGGCCCCCAAGCTGCTGATCTACGACGCCAGCAC |
| | CAGGGCCACCGGCGTGCCCAGCAGGTTCAGCGGCAGCGGCAGCGGCACCGACT |
| | TCACCCTGACCATCAGCAGCCTGCAGCCCGAGGACTTCGCCACCTACTACTGCC |
| | AGCAGAGCTACAGCTACCCCTTCACCTTCGGCCAGGGCACCAAGGTGGAGATC |
| | AAG (SEQ ID NO: 917) |
| V_{L} Amino Acid | DIQMTQSPSSLSASVGDRVTITCRASQSVGNYLAWYQQKPGKAPKLLIYDASTRAT |
| Sequence | GVPSRFSGSGSGTDFTLTISSLQPEDFATYYCQQSYSYPFTFGQGTKVEIK (SEQ ID |
| | NO: 918) |
| Linker Amino Acid | GGGGSGGGGSGGGGS (SEQ ID NO: 361) |
| Sequence | |
| V_{H} Nucleotide | GAGGTGCAGCTGGTGGAGAGCGGCGGCGGCCTGGTGCAGCCCGGCGGCAGCC |
| Sequence | TGAGGCTGAGCTGCGCCGCCAGCGGCTTCAACATCAAGAGCTACGCCATGAAC |
| | TGGGTGAGGCAGGCCCCCGGCAAGGGCCTGGAGTGGGTGGCCTGGATCAACCC |
| | CGGCAGCGGCAACACCAACTACGCCGACAGCGTGAAGGGCAGGTTCACCATCA |
| | GCGCCGACACCAGCAAGAACACCGCCTACCTGCAGATGAACAGCCTGAGGGCC |
| | GAGGACACCGCCGTGTACTACTGCGCCAGGTACGCCTGGGACCTGGACGCCTT |
| | CGACTACTGGGGCCAGGGCACCCTGGTGACCGTGAGCAGC (SEQ ID NO: 919) |
| V_{H} Amino Acid | EVQLVESGGGLVQPGGSLRLSCAASGFNIKSYAMNWVRQAPGKGLEWVAWINPG |
| Sequence | SGNTNYADSVKGRFTISADTSKNTAYLQMNSLRAEDTAVYYCARYAWDLDAFDY |
| | WGQGTLVTVSS (SEQ ID NO: 920) |
| Clone Nucleotide | GACATCCAGATGACCCAGAGCCCCAGCAGCCTGAGCGCCAGCGTGGGCGACA |
| Sequence | GGGTGACCATCACCTGCAGGGCCAGCCAGAGCGTGGGCAACTACCTGGCCTGG |
| | TACCAGCAGAAGCCCGGCAAGGCCCCCAAGCTGCTGATCTACGACGCCAGCAC |
| | CAGGGCCACCGGCGTGCCCAGCAGGTTCAGCGGCAGCGGCAGCGGCACCGACT |
| | TCACCCTGACCATCAGCAGCCTGCAGCCCGAGGACTTCGCCACCTACTACTGCC |
| | AGCAGAGCTACAGCTACCCCTTCACCTTCGGCCAGGGCACCAAGGTGGAGATC |
| | AAGGGCGGCGGCGGCAGCGGCGGCGGCGGCAGCGGCGGCGGCGGCAGCGAGG |
| | TGCAGCTGGTGGAGAGCGGCGGCGGCCTGGTGCAGCCCGGCGGCAGCCTGAG |
| | GCTGAGCTGCGCCGCCAGCGGCTTCAACATCAAGAGCTACGCCATGAACTGGG |
| | TGAGGCAGGCCCCCGGCAAGGGCCTGGAGTGGGTGGCCTGGATCAACCCCGGC |
| | AGCGGCAACACCAACTACGCCGACAGCGTGAAGGGCAGGTTCACCATCAGCGC |
| | CGACACCAGCAAGAACACCGCCTACCTGCAGATGAACAGCCTGAGGGCCGAG |
| | GACACCGCCGTGTACTACTGCGCCAGGTACGCCTGGGACCTGGACGCCTTCGA |
| | CTACTGGGGCCAGGGCACCCTGGTGACCGTGAGCAGC (SEQ ID NO: 921) |

### Fusion Proteins and Conjugates

In some embodiments, the disclosure provides fusion proteins comprising (i) one or more single domain antibodies, or antigen-binding fragments thereof, described herein (e.g., comprising one or more CDRs described herein), and (ii) one or more additional polypeptides. In some embodiments, the disclosure provides fusion proteins comprising (i) one or more single domain antibodies, or antigen-binding fragments thereof, described herein (e.g., comprising one or more CDRs described herein), and (ii) one or more additional domains. For example, a fusion protein can include one or more single domain antibodies described herein and one or more (e.g., 1, 2, 3, 4 or more) constant regions or an Fc region. In some embodiments, one or more single domain antibodies, or antigen-binding fragments thereof, described herein (e.g., one or more CDRs described herein) can be conjugated non-covalently or covalently, e.g., fused, to an antigen (e.g., an antigen target for a cellular therapeutic, e.g., a CAR-T cell or an antibody drug conjugate) as described in, e.g., PCT Publication Nos. WO2017/075537, WO2017/075533, WO2018156802, and WO2018156791.

In some embodiments, the disclosure provides a fusion protein comprising one or more VHH as described herein and one or more additional polypeptides or polypeptide domains. In some embodiments, an additional polypeptide comprises an additional antibody or fragment thereof. Additional antibodies include, e.g., intact IgG, IgE and IgM, bi- or multi-specific antibodies (e.g., Zybodies®, etc), single chain Fvs, polypeptide-Fc fusions, Fabs, cameloid antibodies, masked antibodies (e.g., Probodies®), Small Modular ImmunoPharmaceuticals (“SMIPs™”), single chain or Tandem diabodies (TandAb®), VHHs (including but not limited to those described in the present disclosure), Anticalins®, Nanobodies®, minibodies, BiTE®s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, and Centyrins®.

In some embodiments, the one or more additional polypeptides or polypeptide domains comprises a second antigen-binding domain, such as a second antigen-binding domain that binds to the same target antigen (i.e., CLL-1), for example any of the anti-CLL-1 antibody, or antigen-binding fragments thereof, described herein. In some embodiments, the one or more additional polypeptides or polypeptide domains comprises a second antigen-binding domain, such as a second antigen-binding domain that binds to a different target antigen (e.g., not an epitope of CLL-1).

In some embodiments, an antibody of the disclosure can be covalently attached by linker (e.g., by a disulfide or non-cleavable thioether linker) to a drug (e.g., a cytotoxic agent, such as a toxin) as an antibody drug conjugate (ADC). The drug to which the antibody is covalently attached may have cytotoxic or cytostatic effect when it is not conjugated to the antibody. The ADC can be used to selectively delivery an effective dose of a cytotoxic agent to cells (e.g., to tumor tissue). The ADC may improve the bioavailability of the drug and/or the antibody compared to when the drug and/or antibody is administered in its unconjugated form.

A variety of linker types and strategies are known in the art and any or all of these are contemplated for use with the antibodies or ADCs of the disclosure. In some embodiments, the linker is biodegradable, e.g., cleavable by an endogenous protease (e.g., present in the target tissue and/or cells). In some embodiments the linker comprises a protease cleavable site. In some embodiments, the linker comprises a pH sensitive site, e.g., a site sensitive to acidic pH, e.g., that hydrolyzes under acidic conditions. In some embodiments, the linker is stable under physiological conditions, e.g., sufficiently stable so that the antibody targets the drug to the target tissue prior to release of the drug. In some embodiments, the linker comprises a disulfide bond, e.g., a glutathione-sensitive disulfide bond. In some embodiments, the drug conjugated to the antibody is active only after cleavage of the linker. In some embodiments, the drug conjugated to the antibody is active only after proteolytic digestion of the antibody (e.g., in the lysosome of a target cell). In some embodiments, the linker is a non-cleavable heterobifunctional thiooether linker, e.g., a maleimide linker, e.g., comprising N-hydroxysuccinimide ester (succinimidyl-4-(N-maleimidomethyl) cyclohexane-1-carboxylate or SMCC.

A variety of drugs compatible with ADCs of the disclosure are known in the art and any or all of these are contemplated for use with the antibodies of the disclosure.

Also within the scope of the present disclosure are chimeric antigen receptors (CARs) comprising any of the anti-CLL-1 antibodies, or antigen-binding fragments thereof, described herein. A CAR is an artificially constructed hybrid protein or polypeptide containing the antigen-binding domain of one or more antibodies (e.g., single chain variable fragment (scFv)) linked to T-cell signaling domains. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains. The phrases “antigen(ic) specificity” and “elicit antigen-specific response,” as used herein, means that the CAR can specifically bind to and immunologically recognize antigen, such that binding of the CAR to the antigen elicits an immune response.

Of the conventional CARs containing an antigen-binding domain of an antibody, there are three generations of CARs. “First generation” CARs are typically composed of an extracellular antigen-binding domain (e.g., a scFv), which is fused to a transmembrane domain, which is fused to cytoplasmic/intracellular signaling domain. First generation CARs can provide de novo antigen recognition and cause activation of both CD4+ and CD8+ T cells through their CD3ζ chain signaling domain in a single fusion molecule, independent of HLA-mediated antigen presentation. “Second generation” CARs add an intracellular signaling domain from various co-stimulatory signaling molecules (e.g., CD28, 4-1BB, ICOS, 0X40, CD27, CD40/My88 and NKGD2) to the cytoplasmic tail of the CAR to provide additional signals to the T cell. Second generation CARs comprise those that provide both co-stimulation (e.g., CD28 or 4-1BB) and activation (CD3ζ). “Third generation” CARs comprise those that provide multiple co-stimulatory domains (e.g., CD28 and 4-1BB) and a signaling domain providing activation (e.g., CD3ζ).

The CARs described herein comprise an extracellular portion of the CAR containing anti-CLL-1 binding fragment, a transmembrane domain, and a signaling domain. In some embodiments, the CAR further comprises one or more of a linker region, hinge region, and co-stimulatory signaling domains. In some embodiments, the CAR further comprises a signal peptide/signal sequence.

A CAR can consist of or consist essentially of the specified amino acid sequence or sequences described herein, such that other components, e.g., other amino acids, do not materially change the biological activity of the functional variant.

CARs of the present disclosure (including functional portions and functional variants) can be of any length, i.e., can comprise any number of amino acids, provided that the CARs (or functional portions or functional variants thereof) retain their biological activity. e.g., the ability to specifically bind to the target antigen (e.g., CLL-1), detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

In some embodiments, CAR constructs (including functional portions and functional variants of the invention) can comprise synthetic amino acids in place of one or more naturally-occurring amino acids. Such synthetic amino acids are known in the art, and include, for example, aminocyclohexane carboxylic acid, norleucine, a-amino n-decanoic acid, homoserine. S-acetylaminomethyl-cysteine, trans-3- and trans-4-hydroxyproline, 4-aminophenylalanine, 4-nitrophenylalanine, 4-chlorophenylalanine, 4-carboxyphenylalanine, b-phenylserine b-hydroxyphenylalanine, phenylglycine, a-naphthylalanine, cyclohexylalanine, cyclohexylglycine, indoline-2-carboxylic acid, 1, 2, 3, 4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, a-aminocyclopentane carboxylic acid, a-aminocyclohexane carboxylic acid, a-aminocycloheptane carboxylic acid, a-(2-amino-2-norbornane)-carboxylic acid, a,g-diaminobutyric acid, a,b-diaminopropionic acid, homophenylalanine, and a-tert-butylglycine.

In some embodiments, CAR constructs (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

In some embodiments, CAR constructs (including functional portions and functional variants thereof) can be obtained by methods known in the art. In some embodiments, CAR constructs may be made by any suitable method of making polypeptides or proteins, including de novo synthesis. CAR constructs can be recombinantly produced using the nucleic acids described herein using standard recombinant methods. See, for instance, Green et al., Molecular Cloning: A Laboratory Manual, 4th ed., Cold Spring Harbor Press, Cold Spring Harbor, NY 2012. Further, portions of some of the CAR constructs described herein (including functional portions and functional variants thereof) can be isolated and/or purified from a source, such as a plant, a bacterium, an insect, a mammal, e.g., a rat, a human, etc. Methods of isolation and purification are well known in the art. Alternatively, the CAR constructs described herein (including functional portions and functional variants thereof) can be commercially synthesized by companies, such as Synpep (Dublin, CA), Peptide Technologies Corp. (Gaithersburg, MD), and Multiple Peptide Systems (San Diego. CA). In this respect, the CAR constructs can be synthetic, recombinant, isolated, and/or purified.

Further provided herein are nucleic acids comprising a nucleotide sequence encoding any of the CAR constructs described herein (including functional portions and functional variants thereof). The nucleic acids described herein may comprise a nucleotide sequence encoding any of the leader sequences (e.g., signal peptides), antigen binding domains, transmembrane domains, linker regions, costimulatory signaling domains, and/or intracellular T cell signaling domains described herein.

In some aspects, any of the antigen-binding domains described herein may be operably linked to another domain of the CAR, such as the transmembrane domain or the intracellular domain, for expression in the cell. In some embodiments, a nucleic acid encoding the antigen-binding domain is operably linked to a nucleic acid encoding a transmembrane domain and a nucleic acid encoding an intracellular domain.

In some embodiments, a nucleic acid encoding the anti-CLL-1 antigen binding domain is operably linked to a nucleic acid encoding a linker region, a nucleic acid encoding a transmembrane domain, and/or a nucleic acid encoding an intracellular domain (e.g., a costimulatory signaling domain, a signaling domain). In some embodiments, the CAR comprises any of the anti-CLL-1 antibodies or antigen-binding fragments thereof, described herein (e.g., comprising one or more CDRs described herein). In some embodiments, the CAR comprises any of the anti-CLL-1 antibodies or antigen-binding fragments thereof, provided in any one of SEQ ID NOs: 15, 29, 43, 57, 71, 85, 99, 113, 127, 141, 155, 169, 309, 323, 337, 351, 365, 376-417, or 922-928 or comprising any one of SEQ ID NOs: 2-7, 9, 11, 13, 15, 16-21, 23, 25, 27, 29, 30-35, 37, 39, 41, 43, 44-49, 51, 53, 55, 57, 58-63, 65, 67, 69, 71, 72-77, 79, 81, 83, 85, 86-91, 93, 95, 97, 99, 100-105, 107, 109, 111, 113, 114-119, 121, 123, 125, 127, 128-133, 135, 137, 139, 141, 142-147, 149, 151, 153, 155, 156-161, 163, 165, 167, 169, 170-172, 174, 175-177, 179, 180-182, 184, 185-187 189, 190-192, 194, 195-197, 199, 200-202, 204, 205-207, 209, 210-212, 214, 215-217, 219, 220-222, 224, 225-227, 229, 230-232, 234, 235-237, 239, 240-242, 244, 245-247, 249, 250-252, 254, 255-257, 259, 260-262, 264, 265-267, 269, 271-273, 275, 276-278, 280, 281-283, 285, 286-288, 290, 291-293, 295, 296-301, 303, 305, 307, 309, 310-315, 317, 319, 321, 323, 324-329, 331, 333, 335, 337, 338-343, 345, 347, 349, 351, 352-357, 359, 361, 363, 365-369, 418, 422, 426, 430, 434, 438, 442, 446, 450, 454, 458, 462, 466, 470, 474, 478, 482, 486, 490, 494, 498, 502, 506, 510, 514, 518, 522, 526, 530, 534, 538, 542, 546, 550, 554, 558, 562, 566, 570, 574, 578, 582, 586, 590, 584, 598, 602, 606, 610, 614, 618, 622, 626, 630, 634, 638, 642, 646, 650, 654, 658, 662, 666, 670, 674, 678, 682, 686, 690, 694, 698, 702, 706, 710, 714, 718, 722, 726, 730, 734, 738, 742, 746, 750, 754, 758, 762, 766, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916.

In some embodiments, the CAR comprises a linker region. In some embodiments, the light chain variable region and the heavy chain variable region of the antigen-binding domain can be joined to each other by a linker. In some embodiments, the antigen-binding domain can be joined to another domain, such as a transmembrane domain, hinge, and/or intracellular domain with a linker region. The linker may comprise any suitable amino acid sequence. In some embodiments, the linker is a Gly/Ser linker from about 1 to about 100, from about 3 to about 20, from about 5 to about 30, from about 5 to about 18, or from about 3 to about 8 amino acids in length and consists of glycine and/or serine residues in sequence. Accordingly, the Gly/Ser linker may consist of glycine and/or serine residues. Preferably, the Gly/Ser linker comprises the amino acid sequence of GGGGS (SEQ ID NO: 366), and multiple amino acid sequences of SEQ ID NO: 366 may be present within the linker. Any linker sequence may be used as a spacer between the antigen-binding domain and any other domain of the CAR, such as the transmembrane domain. In some, embodiments, the region linker is ([G]x[S]y)z, for example wherein x can be 1-10, y can be 1-3, and z can be 1-5. In some embodiments, the linker region comprises the amino acid sequence GGGGSGGGGS (SEQ ID NO: 367). In some embodiments, the linker region comprises the amino acid sequence GGGGSGGGGSGGGGS (SEQ ID NO: 368). In some embodiments, the linker region comprises an amino acid sequence of any one of SEQ ID NOs: 11, 25, 39, 53, 67, 81, 95, 109, 123, 137, 151, 165, 305, 319, 333, 347, 361, or 366-369.

In some embodiments, the antigen-binding domain comprises one or more leader sequences (signal peptides, signal sequence), such as those described herein. In some embodiments, the leader sequence may be positioned at the amino terminus of the CAR within the CAR construct. The leader sequence may comprise any suitable leader sequence, e.g., any CARs described herein may comprise any leader sequence, such as those described herein. In some embodiments, while the leader sequence may facilitate expression of the released CARs on the surface of the cell, the presence of the leader sequence in an expressed CAR is not necessary in order for the CAR to function. In some embodiments, upon expression of the CAR on the cell surface, the leader sequence may be cleaved off. Accordingly, in some embodiments, the released CARs (e.g., surface expressed) lack a leader sequence. In some embodiments, the CARs within the CAR construct lack a leader sequence.

### Hinge

In some embodiments, the CAR comprises a hinge/spacer region that links the extracellular antigen-binding domain to another domain, such as a transmembrane domain. The hinge/spacer region can be flexible enough to allow the antigen-binding domain to orient in different directions to facilitate target antigen recognition. In some embodiments, the hinge domain is a portion of the hinge domain of CD8α or CD28, e.g., a fragment containing at least 15 (e.g., 20, 25, 30, 35, or 40) consecutive amino acids of the hinge domain of CD8α or CD28.

In some embodiments, the CAR comprises a hinge domain, such as a hinge domain from CD8, CD28, or IgG4. In some embodiments, the hinge domain is a CD8 (e.g., CD8α) hinge domain. In some embodiments, the CD8 hinge domain is human (e.g., obtained from/derived from a human protein sequence). In some embodiments, the CD8 hinge domain comprises, consists of, or consists essentially of SEQ ID NO: 369.

CD8 hinge region
[SEQ ID NO: 369]
TTTPAPRPPTPAPTIASQPLSLRPEACRPAAGGAVHTRGLDFACD

In some embodiments, the hinge domain is a CD28 hinge domain. In some embodiments, the CD28 hinge domain is human (e.g., obtained from/derived from a human protein sequence). In some embodiments, the CD28 hinge domain comprises, consists of, or consists essentially of SEQ ID NO: 370.

CD28 hinge region
[SEQ ID NO: 370]
AAAIEVMYPPPYLDNEKSNGTIIHVKGKHLCPSPLFPGPSKP

Hinge domains of antibodies, such as an IgG, IgA, IgM, IgE, or IgD antibody, are also compatible for use in the chimeric receptors described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody. In some embodiments, the hinge domain is an IgG4 hinge domain.

Also within the scope of the present disclosure are CARs comprising a hinge domain that is a non-naturally occurring peptide. In some embodiments, the hinge domain between the C-terminus of the extracellular ligand-binding domain of an Fc receptor and the N-terminus of the transmembrane domain is a peptide linker, such as a (GlyxSer)n linker, wherein x and n, independently can be an integer between 3 and 12, including 3, 4, 5, 6, 7, 8, 9, 10, 11, 12, or more. In some embodiments, the linker region comprises an amino acid sequence of any one of SEQ ID NOs: 11, 25, 39, 53, 67, 81, 95, 109, 123, 137, 151, 165, 305, 319, 333, 347, 361, or 366-369.

Additional peptide linkers that may be used in a hinge domain of the chimeric receptors described herein are known in the art. See, e.g., Wriggers et al. Current Trends in Peptide Science (2005) 80(6): 736-74 and PCT Publication No. WO 2012/088461.

In some embodiments, the hinge/spacer region of a presently disclosed CAR comprises a native or modified hinge region of a CD28 polypeptide as described herein. In certain embodiments, the hinge/spacer region of a presently disclosed CAR construct comprises a native or modified hinge region of a CD8α polypeptide as described herein. In certain embodiments, the hinge/spacer region of a presently disclosed CAR construct comprises a native or modified hinge region of a IgG4 polypeptide as described herein.

### Transmembrane Domain

With respect to the transmembrane domain, a CAR can be designed to comprise a transmembrane domain that connects the antigen-binding domain of the CAR to an intracellular region of the CAR. In some embodiments, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of the alpha, beta or zeta chain of the T-cell receptor, CD28, CD3 epsilon, CD45, CD4, CD5, CD8, CD8α, CD9, CD 16, CD22, CD33, CD37, CD64, CD80, CD86, CD134, CD137, CD154, Toll-like receptor 1 (TLR1), TLR2, TLR3, TLR4, TLR5, TLR6, TLR7, TLR8, and TLR9.

In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

In some embodiments, the transmembrane domain is a CD8 (e.g., CD8α) transmembrane domain. In some embodiments, the CD8 transmembrane domain is human (e.g., obtained from/derived from a human protein sequence). In some embodiments, a CD8 transmembrane domain comprises, consists of, or consists essentially of SEQ ID NO: 371.

CD8 transmembrane region
[SEQ ID NO: 371]
IYIWAPLAGTCGVLLLSLVITLYC

In some embodiments, the transmembrane domain is a CD28 transmembrane domain. In some embodiments, the CD28 transmembrane domain is human (e.g., obtained from/derived from a human protein sequence). In some embodiments, the CD28 transmembrane domain comprises, consists of, or consists essentially of SEQ ID NO: 372.

CD28 transmembrane domain
[SEQ ID NO: 372]
FWVLVVVGGVLACYSLLVTVAFIIFWVRSKRSRLLHSDYMNMTPRRPGP
TRKHYQPYAPPRDFAAYRS

### Intracellular Signaling Domains

In some embodiments, the CAR construct comprises an intracellular signaling domain, which may be comprised of one or more signaling domains and costimulatory domains. The intracellular signaling domain of the CAR, is involved in activation of the cell in which the CAR is expressed. In some embodiments, the intracellular signaling domain of the CAR construct described herein is involved in activation of a T lymphocyte or NK cells. In some embodiments, the signaling domain of the CAR construct described herein includes a domain involved in signal activation and/or transduction.

Examples of an intracellular signaling domains for use in the CAR constructs described herein include, but are not limited to, the cytoplasmic portion of a surface receptor, co-stimulatory molecule, and any molecule that acts in concert to initiate signal transduction in a cell (e.g., an immune cell (e.g., a T lymphocyte), NK cell), as well as any derivative or variant of these elements and any synthetic sequence that has the same functional capability.

Examples of the signaling domains that may be used in the intracellular signaling domain of the CARs described herein include, without limitation, a fragment or domain from one or more molecules or receptors including, but are not limited to, TCR, CD3 zeta (CD3ζ), CD3 gamma, CD3 delta, CD3 epsilon, CD86, common FcR gamma, FcR beta (Fc Epsilon Rib), CD79a, CD79b, Fcgamma RIIa, DAP10, DAP 12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1 id, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Any cytoplasmic signaling domain can be used in the CARs described herein. In general, a cytoplasmic signaling domain relays a signal, such as interaction of an extracellular ligand-binding domain with its ligand, to stimulate a cellular response, such as inducing an effector function of the cell (e.g., cytotoxicity).

As will be evident to one of ordinary skill in the art, a factor involved in T cell activation is the phosphorylation of immunoreceptor tyrosine-based activation motif (ITAM) of a cytoplasmic signaling domain. Any ITAM-containing domain known in the art may be used to construct the chimeric receptors described herein, and included as part of the cytoplasmic signaling domain. In general, an ITAM motif may comprise two repeats of the amino acid sequence YxxL/I separated by 6-8 amino acids, wherein each x is independently any amino acid, producing the conserved motif YxxL/Ix(6-8)YxxL/I. In some embodiments, the cytoplasmic signaling domain is from CD3ζ.

CD3ζ associates with TCRs to produce a signal and contains immunoreceptor tyrosine-based activation motifs (ITAMs). In some embodiments, a CD3ζ intracellular T cell signaling sequence is human (e.g., obtained from or derived from a human protein). In some embodiments, a CD3ζ intracellular T cell signaling sequence comprises, consists of, or consists essentially of the amino acid sequence of SEQ ID NO: 373 or 374, or a sequence that is at least 70%, at least 75%, at least 80%, at least 85%, at least 90%, at least 95%, or at least 99% identical the amino acid sequence of SEQ ID NO: 373 or 374. In some embodiments, an intracellular T cell signaling domain comprises a CD3ζ that contains on or more mutated and/or deleted ITAMs.

CD3ζ signaling domain (variant A)
[SEQ ID NO: 373]
RVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR
CD3ζ signaling domain (variant B)
[SEQ ID NO: 374]
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In certain non-limiting embodiments, an intracellular signaling domain of the CAR further comprises at least one (e.g., 1, 2, 3 or more) co-stimulatory signaling domain. In some embodiments, the co-stimulatory signaling domain comprises at least one co-stimulatory molecule, which can provide optimal lymphocyte activation. In general, many immune cells require co-stimulation, in addition to stimulation of an antigen-specific signal, to promote cell proliferation, differentiation and survival, and to activate effector functions of the cell. Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory protein may be compatible for use in the chimeric receptors described herein. The type(s) of co-stimulatory signaling domains may be selected based on factors such as the type of the cells in which the CARs would be expressed (e.g., primary T cells, T cell lines, NK cell lines) and the desired immune effector function (e.g., cytotoxicity).

Examples of such co-stimulatory signaling domains include a fragment or domain from one or more molecules or receptors including, without limitation, 4-1BB, CD28, ICOS, TLR1, TLR2, TLR3, TLR4, TLR5, TLR6, TLR7, TLR8, TLR9, TLR10, TLR11, CD116 receptor beta chain, CSF1-R, LRP1/CD91, SR-A1, SR-A2, MARCO, SR-CL1, SR-CL2, SR-C, SR-E, CR1, CR3, CR4, dectin 1, DEC-205, DC-SIGN, CD14, CD36, LOX-1, CD11b, together with any of the signaling domains listed in the above paragraph in any combination. In some embodiments, the intracellular signaling domain of the CAR includes any portion of one or more co-stimulatory signaling molecules, such as at least one signaling domain from CD3, Fc epsilon RI gamma chain, any derivative or variant thereof, including any synthetic sequence thereof that has the same functional capability, and any combination thereof.

In some embodiments, one or more co-stimulatory signaling domains (e.g., 1, 2, 3, or more) are included in a CAR construct with a CD3 intracellular T cell signaling sequence. In some embodiments, the one or more co-stimulatory signaling domains are selected from CD137 (4-1BB) and CD28, or a combination thereof. In some embodiments, the CAR comprises a 4-1BB (CD137) costimulatory signaling domain. In some embodiments, the CAR comprises a CD28 costimulatory signaling domain. In some embodiments, the CAR comprises both a 4-1BB costimulatory signaling domain and a CD28 costimulatory signaling domain.

4-1BB, also known as CD137, transmits a potent costimulatory signal to T cells, promoting differentiation and enhancing long-term survival of T lymphocytes. In some embodiments, a 4-1BB intracellular signaling sequence is human (e.g., obtained from/derived from a human protein sequence). In some embodiments, the 4-1BB intracellular T cell signaling sequence comprises, consists of, or consists essentially of the amino acid sequence of SEQ ID NO: 375. In some embodiments, the 4-1BB costimulatory signaling domain comprises, consists of, or consists essentially of the amino acid sequence of SEQ ID NO: 375, or a sequence that is at least 70%, at least 75%, at least 80%, at least 85%, at least 90%, at least 95%, or at least 99% identical the amino acid sequence of SEQ ID NO: 375.

4-1BB costimulatory signaling domain
[SEQ ID NO: 375]
KRGRKKLLYIFKQPFMRPVQTTQEEDGCSCRFPEEEEGGCEL

Some suitable costimulatory domains are provided herein, and other suitable costimulatory domains and costimulatory domain sequences will be apparent to the skilled artisan based on the present disclosure in view of the knowledge in the art. Suitable costimulatory domains include, for example, those described in Weinkove et al., Selecting costimulatory domains for chimeric antigen receptors: functional and clinical considerations, Clin Transl Immunology (2019) 8(5): e1049, the entire contents of which are incorporated herein by reference.

Between the antigen-binding domain and the transmembrane domain of the CAR, or between the intracellular signaling domain and the transmembrane domain of the CAR, a spacer domain may be incorporated. As used herein, the term “spacer domain” generally means any oligo- or polypeptide that functions to link the transmembrane domain to, either the antigen binding domain or, the intracellular domain in the polypeptide chain. In some embodiments, the spacer domain may comprise up to 300 amino acids, preferably 10 to 100 amino acids and most preferably 25 to 50 amino acids. In some embodiments, a short oligo- or polypeptide linker, preferably between 2 and 10 amino acids in length may form the linkage between the transmembrane domain and the intracellular domain of the CAR. An example of a linker includes a glycine-serine doublet.

### Signal Peptides

In some embodiments, any of the CARs described herein may further comprise a signal peptide (signal sequence). In general, signal peptides are short amino acid sequences that target a polypeptide to a site in a cell. In some embodiments, the signal peptide directs the CAR to the secretory pathway of the cell and will allow for integration and anchoring of the CAR into the lipid bilayer at the cell surface. Signal sequences including signal sequences of naturally occurring proteins or synthetic, non-naturally occurring signal sequences, that are compatible for use in the chimeric receptors described herein will be evident to one of skill in the art.

The CARs described herein may be prepared in constructs with, e.g., self-cleaving peptides, such that the CAR constructs containing anti-CLL-1 CAR components are bicistronic, tricistronic, etc.

Various CAR constructs and numerous elements of CAR constructs (for example, various CLL-1 binding domains, signal peptides, linkers, hinge sequences, transmembrane domains, costimulatory domains, and signaling domains) are disclosed herein, and those of skill in the art will be able to ascertain the sequences of these elements and of additional suitable elements known in the art based on the present disclosure in view of the knowledge in the art. Exemplary CAR element sequences, e.g., for CLL-1 binding domains, signal peptides, linkers, hinge sequences, transmembrane domains, costimulatory domains, and signaling domains, are disclosed, for example, in WO/2016/120218, e.g., throughout the specification and in Tables 1-8, the entire contents of which are incorporated herein by reference.

Any of the anti-CLL-1 antibodies, or antigen-binding fragments thereof, may be used in a CAR construct, with any one or more of the additional components described herein, e.g., hinge, transmembrane domain, co-stimulatory domain, intracellular signaling domain. In some embodiments, the CAR construct comprises a CLL-1 binding domain comprising one or more of the CDR sequences provided by SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916, a CD8α transmembrane domain, a CD8α hinge domain, a CD137 (4-1BB) co-stimulatory domain, and a CD3ζ intracellular signaling domain. In some embodiments, the CAR construct comprises a CLL-1 binding domain comprising three of the CDR sequences provided by SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916 (e.g., a VHH), a CD8α transmembrane domain, a CD8α hinge domain, a CD137 (4-1BB) co-stimulatory domain, and a CD3ζ intracellular signaling domain. In some embodiments, the CAR construct comprises a CLL-1 binding domain comprising six of the CDR sequences provided by SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 809-813, 820-825, 838-843, 850-854, 861-866, 873-877, 884-889, 896-898, 901-903, 906-908, or 911-916 (e.g., a scFv), a CD8α transmembrane domain, a CD8α hinge domain, a CD137 (4-1BB) co-stimulatory domain, and a CD3ζ intracellular signaling domain.

As will be understood by one of ordinary skill in the art, one or more of the domains of the exemplary CAR constructs may be replaced by another domain. For example, the exemplary CAR constructs provided below comprise a CD8α transmembrane domain, a CD8α hinge domain, a CD137 (4-1BB) co-stimulatory domain, and a CD3ζ intracellular signaling domain, any one of the domains (such as the hinge domain, transmembrane domain, co-stimulatory domain, and/or intracellular signaling domain) may be replaced by another domain, such as those described herein.

In some embodiments, a CAR comprises an amino acid sequence that is at least 70%, at least 75%, at least 80%, at least 85%, at least 90%, at least 95%, or at least 99% identical to the amino acid sequence shown in any one of SEQ ID NOs: 376-417, or 922-928. In amino acid sequences of the exemplary CAR constructs described herein, the following annotations may be used: long dashed underline denotes a hinge domain; double underline denotes a transmembrane domain; [symbol] denotes a costimulatory domain; and bold underline denotes an intracellular signaling domain.

### 1. CAR Clone 1

An exemplary CAR construct, as described herein, comprises a CLL-1 binding domain scFv comprising SEQ ID NO: 15, a CD8α transmembrane domain, a CD8α hinge domain, a CD137 (4-1BB) co-stimulatory domain, and a CD3ζ intracellular signaling domain. In some embodiments, a CAR comprises an amino acid sequence shown in SEQ ID NO: 376, or an amino acid sequence that is at least 70%, at least 75%, at least 80%, at least 85%, at least 90%, at least 95%, or at least 99% identical to the amino acid sequence shown in SEQ ID NO: 376.

(SEQ ID NO: 376)
SYVLTQPPSVSVAPGQTARITCGGNNIGSKSVHWYQQKPGQAPVLVVYDDSDRPSGIPERFSGSNSGNT
ATLTISRVEAGDEADYYCQVWDSSSDHPVFGGGGTQLIILGGGGSGGGGSGGGGSQVQLVQSGAEVKKP
GSSVKVSCKASGGTFSSYAISWVRQAPGQGLEWMGRIIPILGIANYAQKFQGRVTITADESTSTAYMEL

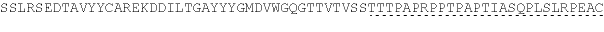

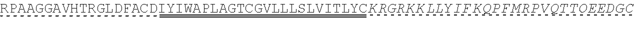

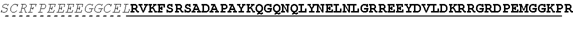

RKNPQEGLYNELQKDKMAEAYSEIGMKGERRRGKGHDGLYQGLSTATKDTYDALHMQALPPR

In some embodiments, a CAR construct as shown in SEQ ID NO: 376 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 2. CAR Clone 2

An exemplary CAR construct, as described herein, comprises a CLL-1 binding domain scFv comprising SEQ ID NO: 29, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 377, or an amino acid sequence that is at least 70%, at least 75%, at least 80%, at least 85%, at least 90%, at least 95%, or at least 99% identical to the amino acid sequence shown in SEQ ID NO: 377.

(SEQ ID NO: 377)
QSVLTQPLSVSVALGQTARITCGGNNIGSKNVHWYQQKAGQAPVLVIQRDSNRPSGIPERFSGSNSGNT
ATLTISRAQAGDEADYFCQVWDSNTYVFGTGTKLTVLGGGGSGGGGSGGGGSEVQLVESGGGLVKPG
GSLRLSCAASGFTFSDYYMSWIRQAPGKGLEWVSYISSSGSTIYYADSVKGRFTISRDNAKNSLYLQMN

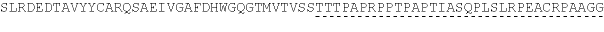

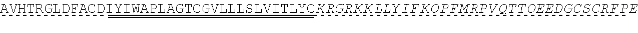

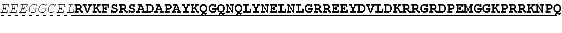

EGLYNELQKDKMAEAYSEIGMKGERRRGKGHDGLYQGLSTATKDTYDALHMQALPPR

In some embodiments, a CAR construct as shown in SEQ ID NO: 377 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 3. CAR Clone 3

An exemplary CAR construct, as described herein, comprises a CLL-1 binding domain scFv comprising SEQ ID NO: 43, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 378, or an amino acid sequence that is at least 70%, at least 75%, at least 80%, at least 85%, at least 90%, at least 95%, or at least 99% identical to the amino acid sequence shown in SEQ ID NO: 378.

(SEQ ID NO: 378)
QSALTQPPSASGSPGQSVTISCTGTSSDVGGYNYVSWYQQHPGKAPKLMIYEVGKRPSGVPDRFSGSKS
GNTASLTVSGLQAEDEADYYCSSYTSSSTRVFGGGTKVTVLGGGGSGGGGSGGGGSEVQLVQSGAEV
KKPGASVKVSCKVSGYTLTELSMHWVRQAPGKGLEWMGGFDPEDGETIYAQKFQGRVTMTEDTSTD

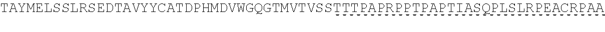

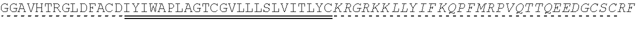

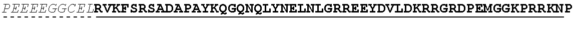

QEGLYNELQKDKMAEAYSEIGMKGERRRGKGHDGLYQGLSTATKDTYDALHMQALPPR

In some embodiments, a CAR construct as shown in SEQ ID NO: 378 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 4. CAR Clone 4

An exemplary CAR construct, as described herein, comprises a CLL-1 binding domain scFv comprising SEQ ID NO: 57, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 379, or an amino acid sequence that is at least 70%, at least 75%, at least 80%, at least 85%, at least 90%, at least 95%, or at least 99% identical to the amino acid sequence shown in SEQ ID NO: 379.

(SEQ ID NO: 379)
QSVVTQPPSVSAAPGQKVTISCSGSSPNIGKNYVSWYQQLPGTAPKLLIYDNNKRPSGIPDRFSGSKSGT
SATLGITGLQTGDEADYYCGTWDSSLSGYVFGTGTKVTVLGGGGSGGGGSGGGGSQVQLVESGGGVV
QPGRSLRLSCAASGFTFSSYGMHWVRQAPGKGLEWVAFISHDGSNKYYADSVKGRSTISRDNSKSTLF

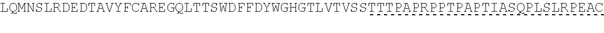

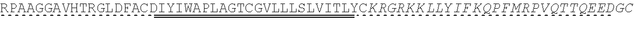

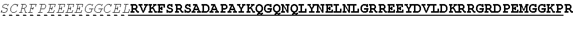

RKNPQEGLYNELQKDKMAEAYSEIGMKGERRRGKGHDGLYQGLSTATKDTYDALHMQALPPR

In some embodiments, a CAR construct as shown in SEQ ID NO: 379 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter)

### 5. CAR Clone 5

An exemplary CAR construct, as described herein, comprises a CLL-1 binding domain scFv comprising SEQ ID NO: 71, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 380, or an amino acid sequence that is at least 70%, at least 75%, at least 80%, at least 85%, at least 90%, at least 95%, or at least 99% identical to the amino acid sequence shown in SEQ ID NO: 380.

(SEQ ID NO: 380)
DIQLTQSPSSLSASVGDRVTITCRASQSISSYLNWYQQKPGKAPKLLIYAASSLQSGVPSRFSGSGSGTDF
TLTISSLQPEDFATYYCQQSYSTPWTFGQGTKVEIKGGGGSGGGGSGGGGSQVQLVESGGGVVQPGRS
LRLSCAASGFTFSSYGMHWVRQAPGKGLEWVAFISHDGSNKYYADSVKGRSTISRDNSKSTLFLQMNS
LRDEDTAVYFCAREGQLTTSWDFFDYWGHGTLVTVSS 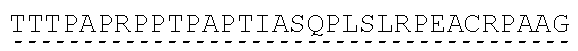 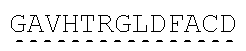
IYIWAPLAGTCGVLLLSLVITLYC 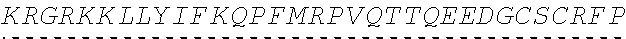 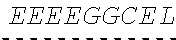
RVKFSRSADAPAYKQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR

In some embodiments, a CAR construct as shown in SEQ ID NO: 380 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 6. CAR Clone 6

An exemplary CAR construct, as described herein, comprises a CLL-1 binding domain scFv comprising SEQ ID NO: 85, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 381, or an amino acid sequence that is at least 70%, at least 75%, at least 80%, at least 85%, at least 90%, at least 95%, or at least 99% identical to the amino acid sequence shown in SEQ ID NO: 381.

(SEQ ID NO: 381)
QSVLTQPPSVSAAPGQKVSIFCSGGSSNIGKNYVSWYQQLPGTAPKLLIYENQHRPSGIPDRFSGSKSGT
SATLGITGLQTGDEADYYCATWDDSLSAGVFGGGTELTVLGGGGSGGGGSGGGGSQLVQSGAEVRKP
GESLKISCKGSGYSFTSYWIGWVRQMPGKGLEWMGFIYPGDSDTRYSPSFQGQVTISADKSISTAYLQR
SSLKASDTAMYYCARLGGYSGYDSEVFYLDYWGQGTTVTVSS 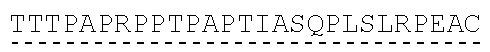 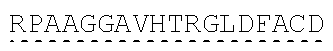
IYIWAPLAGTCGVLLLSLVITLYC 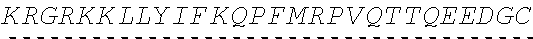 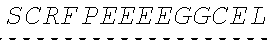
RVKFSRSADAPAYKQGQNQLYNELNLGRREEYDVLDKRRGRDPEMGGKPR
RKNPQEGLYNELOKDKMAEAYSEIGMKGERRRGKGHDGLYQGLSTATKDTYDALHMQALPPR

In some embodiments, a CAR construct as shown in SEQ ID NO: 381 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 7. CAR Clone 7

An exemplary CAR construct, as described herein, comprises a CLL-1 binding domain scFv comprising SEQ ID NO: 99, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 382, or an amino acid sequence that is at least 70%, at least 75%, at least 80%, at least 85%, at least 90%, at least 95%, or at least 99% identical to the amino acid sequence shown in SEQ ID NO: 382.

(SEQ ID NO: 382)
QSALTQPPSASGSPGQSVTISCTGTSSDVGGYNYVSWYQQHPGKAPKLMIYEVGKRPSGVPDRFSGSKS
GNTASLTVSGLQAEDEADYYCSSYTSSSTRVFGGGTKVTVLGGGGSGGGGSGGGGSEVQLVQSGAEV
KKPGASVKVSCKVSGYTLTELSMHWVRQAPGKGLEWMGGFDPEDGETIYAQKFQGRVTMTEDTSTD
TAYMELSSLRSEDTAVYYCATDPHMDVWGQGTMVTVSS 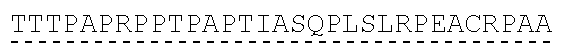 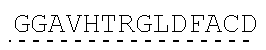
IYIWAPLAGTCGVLLLSLVITLYC 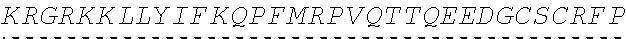 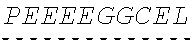
RVKFSRSADAPAYKOGONOLYNELNLGRREEYDVLDKRRGRDPEMGGKPRRKNP
QEGLYNELQKDKMAEAYSEIGMKGERRRGKGHDGLYQGLSTATKDTYDALHMQALPPR

In some embodiments, a CAR construct as shown in SEQ ID NO: 382 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 8. CAR Clone 8

An exemplary CAR construct, as described herein, comprises a CLL-1 binding domain scFv comprising SEQ ID NO: 113, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 383, or an amino acid sequence that is at least 70%, at least 75%, at least 80%, at least 85%, at least 90%, at least 95%, or at least 99% identical to the amino acid sequence shown in SEQ ID NO: 383.

(SEQ ID NO: 383)
DIQMTQSPSSLSASVGDRVTITCRASQSISSYLNWYQQKPGKAPKLLIYAASSLQSGVPSRFSGSGSGTD
FTLTISSLQPEDFATYYCQQSYSTPLTFGGGTKVEIKGGGGSGGGGSGGGGSQVQLVQSGAEVKKPGSS
VKVSCKASGGTFTSYGISWVRQAPGQGLEWMGWISAYNGNTNYAQKLQGRVTMTTDTSTSTAYMEL
RSLRAEDTAVYYCALPTADDAFDIWGQGTTVTVSS 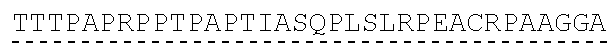 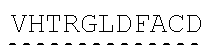
IYIWAPLAGTCGVLLLSLVITLYC 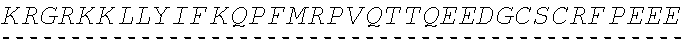 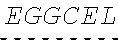
RVKFSRSADAPAYKQGQNQLYNELNLGRREEYDVLDKRRGRDPEMGGKPRRKNPQEG
LYNELQKDKMAEAYSEIGMKGERRRGKGHDGLYOGLSTATKDTYDALHMQALPPR

In some embodiments, a CAR construct as shown in SEQ ID NO: 383 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 9. CAR Clone 9

An exemplary CAR construct, as described herein, comprises a CLL-1 binding domain scFv comprising SEQ ID NO: 127, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 384, or an amino acid sequence that is at least 70%, at least 75%, at least 80%, at least 85%, at least 90%, at least 95%, or at least 99% identical to the amino acid sequence shown in SEQ ID NO: 384.

(SEQ ID NO: 384)
SYELTQPPSVSVAPGKTARITCGGNNIGSKSVHWYQQKPGQAPVLVVYDDSDRPSGIPERFSGSNSGNT
ATLTISRVEAGDEADYYCQVWDSGSDHPVVSGGGTQLIILGGGGSGGGGSGGGGSQVQLVESGGGLV
QPGGSLRLSCAASGFTFSSYAMSWVRQAPGKGLEWVSAIGGSGGSTYYADSVKGRFTISRDNSKNTLY
LQMNSLRAEDTAVYYCANIDGTFWGQGTTVTVSS 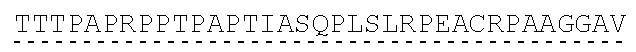 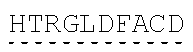
IYIWAPLAGTCGVLLLSLVITLYC 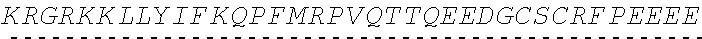 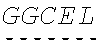
RVKFSRSADAPAYKQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR

In some embodiments, a CAR construct as shown in SEQ ID NO: 384 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 10. CAR Clone 10

An exemplary CAR construct, as described herein, comprises a CLL-1 binding domain scFv comprising SEQ ID NO: 141, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 385, or an amino acid sequence that is at least 70%, at least 75%, at least 80%, at least 85%, at least 90%, at least 95%, or at least 99% identical to the amino acid sequence shown in SEQ ID NO: 385.

(SEQ ID NO: 385)
DIQMTQSPSFLSASVGDRVTITCRASQGISSYLAWYQQKPGKAPKLLIYSASSLQSGVPSRFSGSGSGTD
FTLTISSLOPEDFATYYCQQSYNSPTFGQGTRLEIKGGGGSGGGGSGGGGSQVQLVQSGAEVKKPGASV
KVSCKASGYTFTGYYMHWVRQAPGQGLEWMGWMNPNSGNTGYAQKFQGRVTMTRNTSISTAYLEL
TSLTSDDTAVYYCARGDTGDFFDHWGQGTLVTVSS 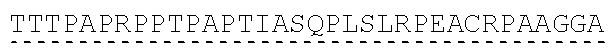 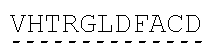
IYIWAPLAGTCGVLLLSLVITLYC 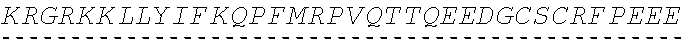 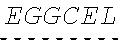
RVKFSRSADAPAYKQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR

In some embodiments, a CAR construct as shown in SEQ ID NO: 385 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 11. CAR Clone 11

An exemplary CAR construct, as described herein, comprises a CLL-1 binding domain scFv comprising SEQ ID NO: 155, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 386, or an amino acid sequence that is at least 70%, at least 75%, at least 80%, at least 85%, at least 90%, at least 95%, or at least 99% identical to the amino acid sequence shown in SEQ ID NO: 386.

(SEQ ID NO: 386)
QSVLTQPASVSGSPGQSITISCTGTSSDVGASNSVSWYQRHPGKPPKLMIYDVSNRPSGISNRFSGSKSG
NTASLTISGLQSEDEADYYCSSYTGSSTFVVFGGGTQLIILGGGGSGGGGSGGGGSEVQLVESGGGVVQ
PGGSLRLSCAASGFTFSSYWMSWVRQAPGKGLEWVANIRQDGSEKYYVDSVKGRFTISRDNAKNSLY
LQMNSLRAEDAAVYYCAREQDTAMVAFDYWGQGTLVTVSS 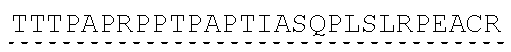 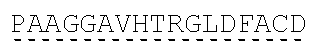
IYIWAPLAGTCGVLLLSLVITLYC 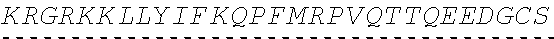 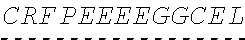
RVKFSRSADAPAYKOGONOLYNELNLGRREEYDVLDKRRGRDPEMGGKPRR
KNPQEGLYNELQKDKMAEAYSEIGMKGERRRGKGHDGLYQGLSTATKDTYDALHMQALPPR

In some embodiments, a CAR construct as shown in SEQ ID NO: 386 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 12. CAR Clone 12

An exemplary CAR construct, as described herein, comprises a CLL-1 binding domain scFv comprising SEQ ID NO: 169, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 387, or an amino acid sequence that is at least 70%, at least 75%, at least 80%, at least 85%, at least 90%, at least 95%, or at least 99% identical to the amino acid sequence shown in SEQ ID NO: 387.

(SEQ ID NO: 387)
DIQMTQSPSSLSASVGDRVTITCQASQDISNYLNWYQQKPGKAPKLLIYDASNLETGVPSRFSGSGSGT
DFTLTISSPQPEDLATYYCQQANSFPFTFGQGTKVEIKGGGGSGGGGSGGGGSQVQLVQSGAEVKKPGS
SVKVSCKASGYSFTRNDITWVRQANGRGLEWLGRLNPNNGDTVYAEVFQGRVAMTRDTSTDTAYME
LASLKSDDTAVYFCARGFWSFDVWGQGTMVTVSS 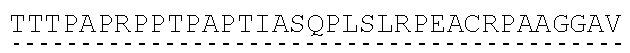 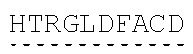
IYIWAPLAGTCGVLLLSLVITLYC 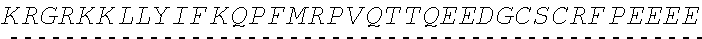 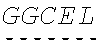
RVKFSRSADAPAYKQGQNQLYNELNLGRREEYDVLDKRRGRDPEMGGKPRRKNPQEGL
YNELQKDKMAEAYSEIGMKGERRRGKGHDGLYOGLSTATKDTYDALHMQALPPR

In some embodiments, a CAR construct as shown in SEQ ID NO: 387 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 13. CAR Clone 13

An exemplary CAR construct, as described herein, comprises a CLL-1 binding domain VHH comprising SEQ ID NO: 174, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 388, or an amino acid sequence that is at least 70%, at least 75%, at least 80%, at least 85%, at least 90%, at least 95%, or at least 99% identical to the amino acid sequence shown in SEQ ID NO: 388.

(SEQ ID NO: 388)
QVQLVQSGGGLVQPGGSLRLSCAASSFAFSAYEMSWVRQAPGQRLEWVSGITGSGGTTYYEDSVKGR
IFTISRDNSKNTLYLQMNTLRAEDTAMYYCVSHLGGTYGSGFSWGQGTLVTVSSS 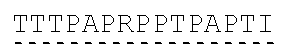 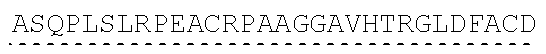
IYIWAPLAGTCGVLLLSLVITLYC 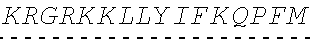 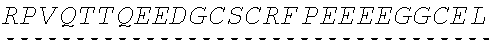
RVKFSRSADAPAYKQGQNQLYNELNLGRREEYDVLDKRRG
RDPEMGGKPRRKNPQEGLYNELQKDKMAEAYSEIGMKGERRRGKGHDGLYQGLSTATKDTYD
ALHMOALPPR

In some embodiments, a CAR construct as shown in SEQ ID NO: 388 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 14. CAR Clone 14

An exemplary CAR construct, as described herein, comprises a CLL-1 binding domain VHH comprising SEQ ID NO: 179, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 389, or an amino acid sequence that is at least 70%, at least 75%, at least 80%, at least 85%, at least 90%, at least 95%, or at least 99% identical to the amino acid sequence shown in SEQ ID NO: 389.

(SEQ ID NO: 389)
QVQLVQSGGGLVQPGGSLRLSCAASDFAFDYYEMSWVRQAPGQGLEWVSSISGSGGTTLYADSVKGR
FTISRDNSKNTLYLQMNTLRAEDTALYYCATEDPSSVYWGQGTLVTVSS 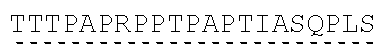 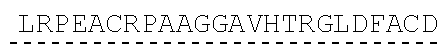
IYIWAPLAGTCGVLLLSLVITLYC 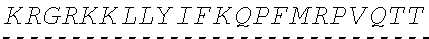 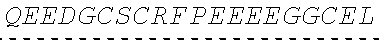
RVKFSRSADAPAYKQGQNQLYNELNLGRREEYDVLDKRRGRDPEM
GGKPRRKNPQEGLYNELQKDKMAEAYSEIGMKGERRRGKGHDGLYQGLSTATKDTYDALHMQ
ALPPR

In some embodiments, a CAR construct as shown in SEQ ID NO: 389 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 15. CAR Clone 15

An exemplary CAR construct, as described herein, comprises a CLL-1 binding domain VHH comprising SEQ ID NO: 184, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 390, or an amino acid sequence that is at least 70%, at least 75%, at least 80%, at least 85%, at least 90%, at least 95%, or at least 99% identical to the amino acid sequence shown in SEQ ID NO: 390.

(SEQ ID NO: 390)
QVQLVQSGGGLVQPGGSLRLSCAASSFSFSDYEMSWVRQAPGQRLEWVSAISGSGGSTYYADSVKGR
FTISRDNSKNTLYLQMNTLRAEDTATYYCVRVPLGHCSSTDCANPYWGQGTLVTVSS 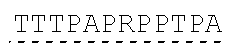 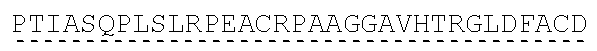
IYIWAPLAGTCGVLLLSLVITLYC 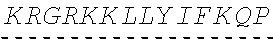 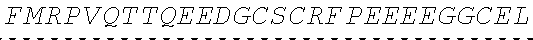
RVKFSRSADAPAYKQGQNQLYNELNLGRREEYDVLDKR
RGRDPEMGGKPRRKNPQEGLYNELQKDKMAEAYSEIGMKGERRRGKGHDGLYQGLSTATKDT
YDALHMQALPPR

In some embodiments, a CAR construct as shown in SEQ ID NO: 390 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 16. CAR Clone 16

An exemplary CAR construct, as described herein, comprises a CLL-1 binding domain VHH comprising SEQ ID NO: 189, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 391, or an amino acid sequence that is at least 70%, at least 75%, at least 80%, at least 85%, at least 90%, at least 95%, or at least 99% identical to the amino acid sequence shown in SEQ ID NO: 391.

(SEQ ID NO: 391)
QVQLVQSGGGLVQPGGSLRLSCAASSFDFADYEMSWVRQAPGQRLEWVSTVSGSGGDTWYADSVKG
RFTISRDNSKNTLYLQMNTLRAEDTAVYYCAKVGPKWELVDWGQGTLVTVSS 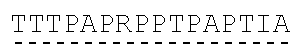 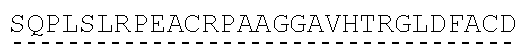
IYIWAPLAGTCGVLLLSLVITLYC 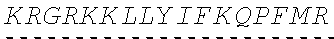 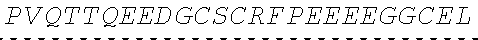
RVKFSRSADAPAYKOGONOLYNELNLGRREEYDVLDKRRGR
DPEMGGKPRRKNPQEGLYNELQKDKMAEAYSEIGMKGERRRGKGHDGLYOGLSTATKDTYDA
LHMQALPPR

In some embodiments, a CAR construct as shown in SEQ ID NO: 391 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 17. CAR Clone 17

An exemplary CAR construct, as described herein, comprises a CLL-1 binding domain VHH comprising SEQ ID NO: 194, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 392, or an amino acid sequence that is at least 70%, at least 75%, at least 80%, at least 85%, at least 90%, at least 95%, or at least 99% identical to the amino acid sequence shown in SEQ ID NO: 392.

(SEQ ID NO: 392)
QVQLVQSGGGLVQPGGSLRLSCAASSFSFSDYEMSWVRQAPGKGLEWVSLISGDGGSTYYADSVKGR
FTISRDNSKNTLYLQMNTLRAEDTAMYYCARDPYYYGSGRITKLDYWGQGTLVTVSS 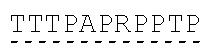 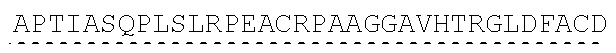
IYIWAPLAGTCGVLLLSLVITLYC 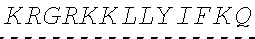 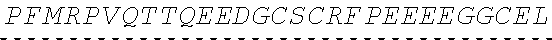
RVKFSRSADAPAYKQGONQLYNELNLGRREEYDVLDK
RRGRDPEMGGKPRRKNPQEGLYNELQKDKMAEAYSEIGMKGERRRGKGHDGLYQGLSTATKD
TYDALHMQALPPR

In some embodiments, a CAR construct as shown in SEQ ID NO: 392 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 18. CAR Clone 18

An exemplary CAR construct, as described herein, comprises a CLL-1 binding domain VHH comprising SEQ ID NO: 199, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 393, or an amino acid sequence that is at least 70%, at least 75%, at least 80%, at least 85%, at least 90%, at least 95%, or at least 99% identical to the amino acid sequence shown in SEQ ID NO: 393.

(SEQ ID NO: 393)
QVQLVQSGGGLVQPGGSLRLSCAASGFSFSTYEMSWVRQAPGQRLEWLSGIIGNSGRTYYSDSVKGRF
TISRDNSKNTLYLQMNTLRAEDTAMYYCARSRGGFDAFDIWGQGTMVTVSS 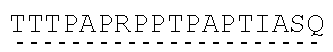 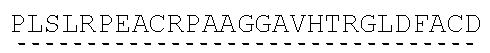
IYIWAPLAGTCGVLLLSLVITLYC 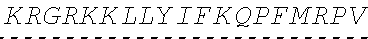 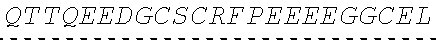
RVKFSRSADAPAYKQGQNQLYNELNLGRREEYDVLDKRRGRDP
EMGGKPRRKNPQEGLYNELQKDKMAEAYSEIGMKGERRRGKGHDGLYQGLSTATKDTYDALH
MQALPPR

In some embodiments, a CAR construct as shown in SEQ ID NO: 393 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 19. CAR Clone 19

An exemplary CAR construct, as described herein, comprises a CLL-1 binding domain VHH comprising SEQ ID NO: 204, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 394, or an amino acid sequence that is at least 70%, at least 75%, at least 80%, at least 85%, at least 90%, at least 95%, or at least 99% identical to the amino acid sequence shown in SEQ ID NO: 394.

(SEQ ID NO:394)
QVQLVQSGGGLVQPGGSLRLSCAASDFSFASYEMSWVRQAPGQGLEWVALIDNGGKTYYADSVKGR
FTISRDNSKNTLYLQMNTLRAEDAALYYCVRDGGVAGPPTWGQGTLVTVSS 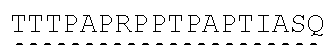 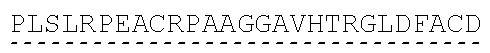
IYIWAPLAGTCGVLLLSLVITLYC 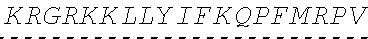 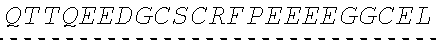
RVKFSRSADAPAYKQGQNQLYNELNLGRREEYDVLDKRRGRDP
EMGGKPRRKNPQEGLYNELQKDKMAEAYSEIGMKGERRRGKGHDGLYQGLSTATKDTYDALH
MQALPPR

In some embodiments, a CAR construct as shown in SEQ ID NO: 394 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 20. CAR Clone 20

An exemplary CAR construct, as described herein, comprises a CLL-1 binding domain VHH comprising SEQ ID NO: 209, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 395, or an amino acid sequence that is at least 70%, at least 75%, at least 80%, at least 85%, at least 90%, at least 95%, or at least 99% identical to the amino acid sequence shown in SEQ ID NO:395.

(SEQ ID NO: 395)
QVQLVQSGGGLVQPGGSLRLSCAASDFSFSYYEMSWVRQAPGKGLEWVSTISGSGDSTYYADSVKGR

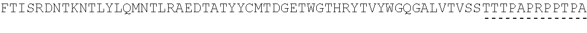

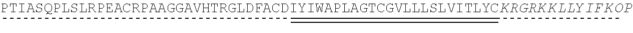

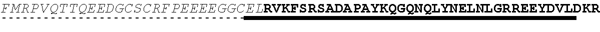

RGRDPEMGGKPRRKNPQEGLYNELQKDKMAEAYSEIGMKGERRRGKGHDGLYQGLSTATKDT
YDALHMQALPPR

In some embodiments, a CAR construct as shown in SEQ ID NO: 395 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 21. CAR Clone 21

An exemplary CAR construct, as described herein, comprises a CLL-1 binding domain VHH comprising SEQ ID NO: 214, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO:396, or an amino acid sequence that is at least 70%, at least 75%, at least 80%, at least 85%, at least 90%, at least 95%, or at least 99% identical to the amino acid sequence shown in SEQ ID NO:396.

(SEQ ID NO: 396)
QVQLVQSGGGLVQPGGSLRLSCAASDFSFYFYEMRWVRQAPGQGLEWVSTISGSGDSTYYADSVKGR

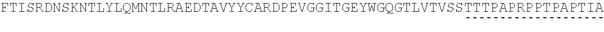

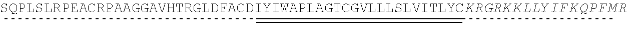

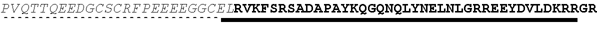

DPEMGGKPRRKNPQEGLYNELQKDKMAEAYSEIGMKGERRRGKGHDGLYQGLSTATKDTYDA
LHMQALPPR

In some embodiments, a CAR construct as shown in SEQ ID NO: 396 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 22. CAR Clone 22

An exemplary CAR construct, as described herein, comprises a CLL-1 binding domain VHH comprising SEQ ID NO: 219, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 397, or an amino acid sequence that is at least 70%, at least 75%, at least 80%, at least 85%, at least 90%, at least 95%, or at least 99% identical to the amino acid sequence shown in SEQ ID NO: 397.

(SEQ ID NO: 397)
QVQLVQSGGGLVQPGGSLRLSCAASAFDFASYEMSWVRQAPGQRLEWVSGISGSGGSTYYADSVKGR

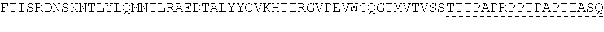

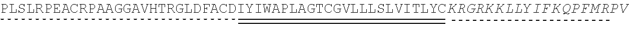

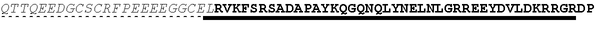

EMGGKPRRKNPQEGLYNELQKDKMAEAYSEIGMKGERRRGKGHDGLYQGLSTATKDTYDALH
MQALPPR

In some embodiments, a CAR construct as shown in SEQ ID NO: 397 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 23. CAR Clone 23

An exemplary CAR construct, as described herein, comprises a CLL-1 binding domain VHH comprising SEQ ID NO: 224, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 398, or an amino acid sequence that is at least 70%, at least 75%, at least 80%, at least 85%, at least 90%, at least 95%, or at least 99% identical to the amino acid sequence shown in SEQ ID NO: 398.

(SEQ ID NO: 398)
QVQLVQSGGGLVQPGGSLRLSCAASYFDFDDYEMSWVRQAPGKGLEWVAVISGGGRDTWYADSVK

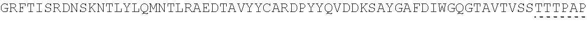

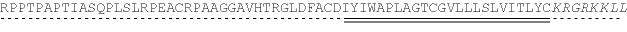

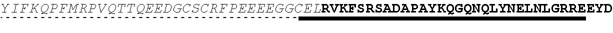

VLDKRRGRDPEMGGKPRRKNPQEGLYNELQKDKMAEAYSEIGMKGERRRGKGHDGLYQGLST
ATKDTYDALHMQALPPR

In some embodiments, a CAR construct as shown in SEQ ID NO: 398 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 24. CAR Clone 24

An exemplary CAR construct, as described herein, comprises a CLL-1 binding domain VHH comprising SEQ ID NO: 229, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 399, or an amino acid sequence that is at least 70%, at least 75%, at least 80%, at least 85%, at least 90%, at least 95%, or at least 99% identical to the amino acid sequence shown in SEQ ID NO: 399.

(SEQ ID NO: 399)
QVQLVQSGGGLVQPGGSLRLSCAASAFDFYDYEMSWVRQAPGQGLEWVSAISNSGDATYYADSVKG

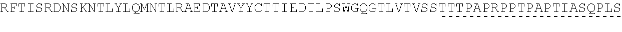

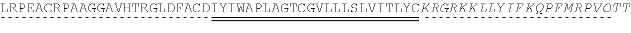

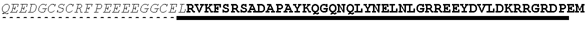

GGKPRRKNPQEGLYNELQKDKMAEAYSEIGMKGERRRGKGHDGLYQGLSTATKDTYDALHMQ
ALPPR

In some embodiments, a CAR construct as shown in SEQ ID NO: 399 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 25. CAR Clone 25

An exemplary CAR construct, as described herein, comprises a CLL-1 binding domain VHH comprising SEQ ID NO: 234, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 400, or an amino acid sequence that is at least 70%, at least 75%, at least 80%, at least 85%, at least 90%, at least 95%, or at least 99% identical to the amino acid sequence shown in SEQ ID NO: 400.

(SEQ ID NO: 400)
QVQLVQSGGGLVQPGGSLRLSCAASSFDFSSYEMSWVRQAPGQGLEWVSLISGSGDSTYYADSVKGRF

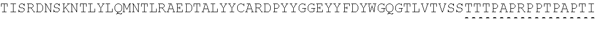

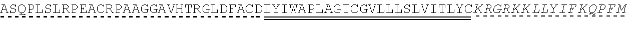

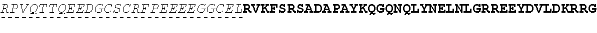

RDPEMGGKPRRKNPQEGLYNELQKDKMAEAYSEIGMKGERRRGKGHDGLYQGLSTATKDTYD
ALHMQALPPR

In some embodiments, a CAR construct as shown in SEQ ID NO: 400 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 26. CAR Clone 26

An exemplary CAR construct, as described herein, comprises a CLL-1 binding domain VHH comprising SEQ ID NO: 239, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 401, or an amino acid sequence that is at least 70%, at least 75%, at least 80%, at least 85%, at least 90%, at least 95%, or at least 99% identical to the amino acid sequence shown in SEQ ID NO: 401.

(SEQ ID NO: 401)
QVQLVQSGGGLVQPGGSLRLSCAASAFSFSSYEMSWVRQAPGQRLEWVAHINGSGGSTYYADSVKGR

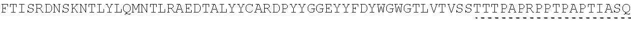

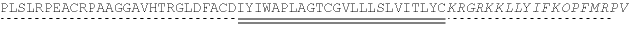

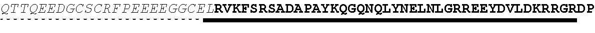

EMGGKPRRKNPQEGLYNELQKDKMAEAYSEIGMKGERRRGKGHDGLYQGLSTATKDTYDALH
MQALPPR

In some embodiments, a CAR construct as shown in SEQ ID NO: 401 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 27. CAR Clone 27

An exemplary CAR construct, as described herein, comprises a CLL-1 binding domain VHH comprising SEQ ID NO: 244, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO:402, or an amino acid sequence that is at least 70%, at least 75%, at least 80%, at least 85%, at least 90%, at least 95%, or at least 99% identical to the amino acid sequence shown in SEQ ID NO:402.

(SEQ ID NO: 402)
QVQLVQSGGGLVQPGGSLRLSCAASDFSFADYEMSWVRQAPGQGLEWVSLISGDGGSTYYADSVKGR

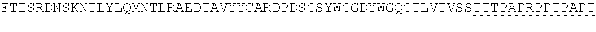

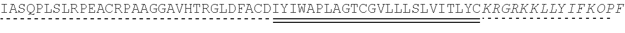

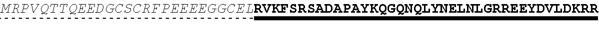

GRDPEMGGKPRRKNPQEGLYNELQKDKMAEAYSEIGMKGERRRGKGHDGLYQGLSTATKDTY
DALHMQALPPR

In some embodiments, a CAR construct as shown in SEQ ID NO: 402 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 28. CAR Clone 28

An exemplary CAR construct, as described herein, comprises a CLL-1 binding domain VHH comprising SEQ ID NO: 249, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO:403, or an amino acid sequence that is at least 70%, at least 75%, at least 80%, at least 85%, at least 90%, at least 95%, or at least 99% identical to the amino acid sequence shown in SEQ ID NO:403.

(SEQ ID NO: 403)
QVQLVQSGGGLVQPGGSLRLSCAASDFYFADYEMSWVRQAPGKGLEWVSLISGDGGSTYYADSVKG

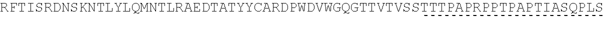

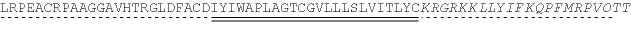

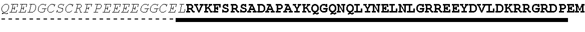

GGKPRRKNPQEGLYNELQKDKMAEAYSEIGMKGERRRGKGHDGLYQGLSTATKDTYDALHMQ
ALPPR

In some embodiments, a CAR construct as shown in SEQ ID NO: 403 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 29. CAR Clone 29

An exemplary CAR construct, as described herein, comprises a CLL-1 binding domain VHH comprising SEQ ID NO: 254, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO:404, or an amino acid sequence that is at least 70%, at least 75%, at least 80%, at least 85%, at least 90%, at least 95%, or at least 99% identical to the amino acid sequence shown in SEQ ID NO:404.

(SEQ ID NO: 404)
QVQLVQSGGGLVQPGGSLRLSCAASAFDFDSYEMSWVRQAPGQRLEWVSTISGHGGNTYYADSVKG

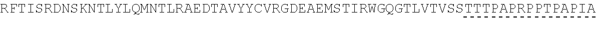

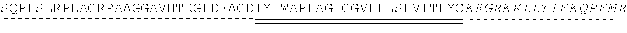

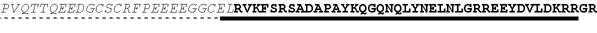

DPEMGGKPRRKNPQEGLYNELQKDKMAEAYSEIGMKGERRRGKGHDGLYQGLSTATKDTYDA
LHMQALPPR

In some embodiments, a CAR construct as shown in SEQ ID NO: 404 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 30. CAR Clone 30

An exemplary CAR construct, as described herein, comprises a CLL-1 binding domain VHH comprising SEQ ID NO: 259, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 405, or an amino acid sequence that is at least 70%, at least 75%, at least 80%, at least 85%, at least 90%, at least 95%, or at least 99% identical to the amino acid sequence shown in SEQ ID NO: 405.

(SEQ ID NO: 405)
QVQLVQSGGGLVQPGGSLRLSCAASDFDFDAYEMSWVRQAPGQGLEWVSVIYSDGSTYYADSVKGR

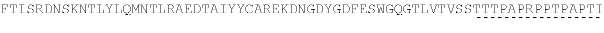

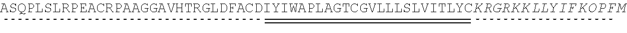

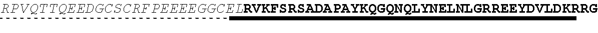

RDPEMGGKPRRKNPQEGLYNELQKDKMAEAYSEIGMKGERRRGKGHDGLYQGLSTATKDTYD
ALHMQALPPR

In some embodiments, a CAR construct as shown in SEQ ID NO: 405 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 31. CAR Clone 31

An exemplary CAR construct, as described herein, comprises a CLL-1 binding domain VHH comprising SEQ ID NO: 264, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 406, or an amino acid sequence that is at least 70%, at least 75%, at least 80%, at least 85%, at least 90%, at least 95%, or at least 99% identical to the amino acid sequence shown in SEQ ID NO: 406.

(SEQ ID NO: 406)
QVQLVQSGGGLVQPGGSLRLSCAASSFAFSDYEMSWVRQAPGQGLEWVSTISGSGGSTYYADSVKGR

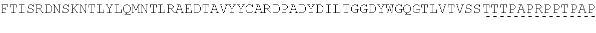

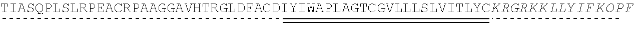

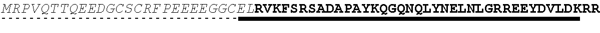

GRDPEMGGKPRRKNPQEGLYNELQKDKMAEAYSEIGMKGERRRGKGHDGLYQGLSTATKDTY
DALHMQALPPR

In some embodiments, a CAR construct as shown in SEQ ID NO: 406 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 32. CAR Clone 32

An exemplary CAR construct, as described herein, comprises a CLL-1 binding domain VHH comprising SEQ ID NO: 269, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 407, or an amino acid sequence that is at least 70%, at least 75%, at least 80%, at least 85%, at least 90%, at least 95%, or at least 99% identical to the amino acid sequence shown in SEQ ID NO: 407.

(SEQ ID NO: 407)
QVQLVQSGGGLVQPGGSLRLSCAASDFDFSDYEMSWVRQAPGKGLEWVSAISDSGDTTYYADSVKGR

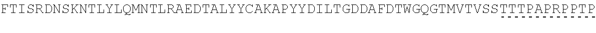

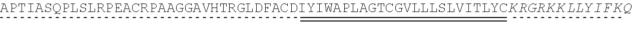

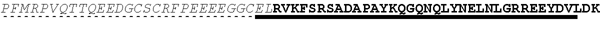

RRGRDPEMGGKPRRKNPQEGLYNELQKDKMAEAYSEIGMKGERRRGKGHDGLYQGLSTATKD
TYDALHMQALPPR

In some embodiments, a CAR construct as shown in SEQ ID NO: 407 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 33. CAR Clone 33

An exemplary CAR construct, as described herein, comprises a CLL-1 binding domain VHH comprising SEQ ID NO: 275, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 408, or an amino acid sequence that is at least 70%, at least 75%, at least 80%, at least 85%, at least 90%, at least 95%, or at least 99% identical to the amino acid sequence shown in SEQ ID NO: 408.

(SEQ ID NO: 408)
QVQLVQSGGGLVQPGGSLRLSCAASYFDFDAYEMSWVRQAPGKGLEWVSLISGDGGSTYYADSVKG

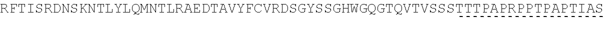

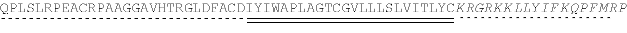

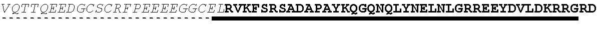

PEMGGKPRRKNPQEGLYNELQKDKMAEAYSEIGMKGERRRGKGHDGLYQGLSTATKDTYDAL
HMQALPPR

In some embodiments, a CAR construct as shown in SEQ ID NO: 408 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 34. CAR Clone 34

An exemplary CAR construct, as described herein, comprises a CLL-1 binding domain VHH comprising SEQ ID NO: 280, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 409, or an amino acid sequence that is at least 70%, at least 75%, at least 80%, at least 85%, at least 90%, at least 95%, or at least 99% identical to the amino acid sequence shown in SEQ ID NO: 409.

(SEQ ID NO: 409)
QVQLVQSGGGLVQPGGSLRLSCAASYFDFAYYEMSWVRQAPGKGLEWVSLISGDGGSTYYADSVKG

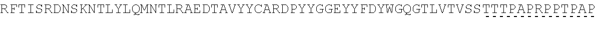

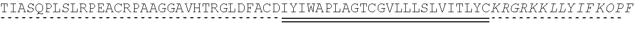

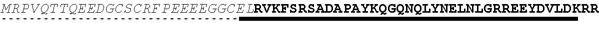

GRDPEMGGKPRRKNPQEGLYNELQKDKMAEAYSEIGMKGERRRGKGHDGLYQGLSTATKDTY
DALHMQALPPR

In some embodiments, a CAR construct as shown in SEQ ID NO: 409 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 35. CAR Clone 35

An exemplary CAR construct, as described herein, comprises a CLL-1 binding domain VHH comprising SEQ ID NO: 285, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 410, or an amino acid sequence that is at least 70%, at least 75%, at least 80%, at least 85%, at least 90%, at least 95%, or at least 99% identical to the amino acid sequence shown in SEQ ID NO: 410.

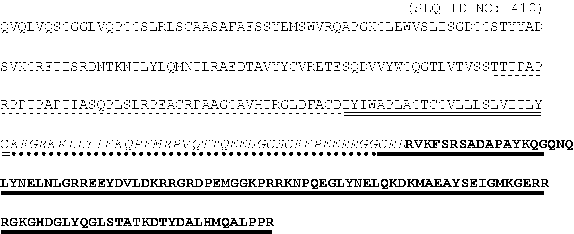

In some embodiments, a CAR construct as shown in SEQ ID NO: 410 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 36. CAR Clone 36

An exemplary CAR construct, as described herein, comprises a CLL-1 binding domain VHH comprising SEQ ID NO: 290, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 411, or an amino acid sequence that is at least 70%, at least 75%, at least 80%, at least 85%, at least 90%, at least 95%, or at least 99% identical to the amino acid sequence shown in SEQ ID NO: 411.

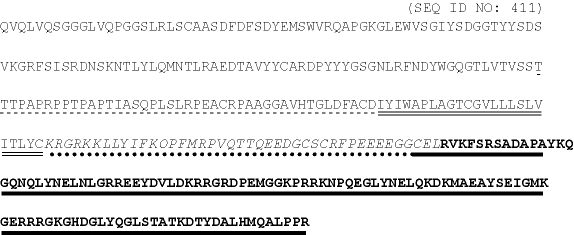

In some embodiments, a CAR construct as shown in SEQ ID NO: 411 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 37. CAR Clone 37

An exemplary CAR construct, as described herein, comprises a CLL-1 binding domain VHH comprising SEQ ID NO: 295, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 412, or an amino acid sequence that is at least 70%, at least 75%, at least 80%, at least 85%, at least 90%, at least 95%, or at least 99% identical to the amino acid sequence shown in SEQ ID NO: 412.

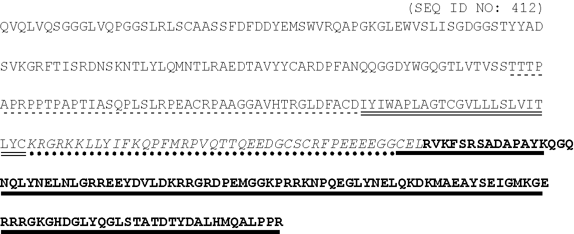

In some embodiments, a CAR construct as shown in SEQ ID NO: 412 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 38. CAR Clone 38

An exemplary CAR construct, as described herein, comprises a CLL-1 binding domain scFv comprising SEQ ID NO: 309, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 413, or an amino acid sequence that is at least 70%, at least 75%, at least 80%, at least 85%, at least 90%, at least 95%, or at least 99% identical to the amino acid sequence shown in SEQ ID NO: 413.

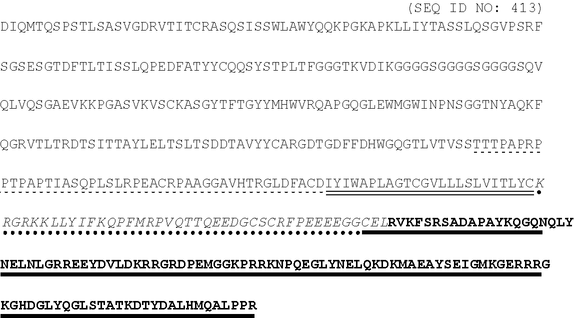

In some embodiments, a CAR construct as shown in SEQ ID NO: 413 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 39. CAR Clone 39

An exemplary CAR construct, as described herein, comprises a CLL-1 binding domain scFv comprising SEQ ID NO: 323, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 414, or an amino acid sequence that is at least 70%, at least 75%, at least 80%, at least 85%, at least 90%, at least 95%, or at least 99% identical to the amino acid sequence shown in SEQ ID NO: 414.

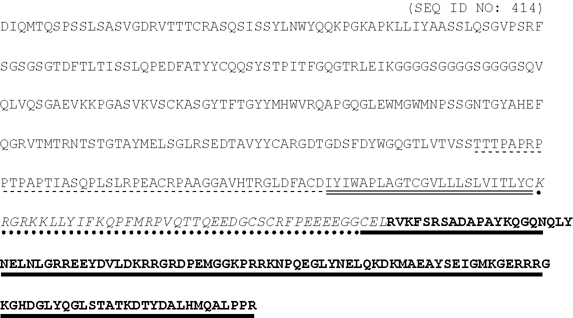

In some embodiments, a CAR construct as shown in SEQ ID NO: 414 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 40. CAR Clone 40

An exemplary CAR construct, as described herein, comprises a CLL-1 binding domain scFv comprising SEQ ID NO: 337, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 415, or an amino acid sequence that is at least 70%, at least 75%, at least 80%, at least 85%, at least 90%, at least 95%, or at least 99% identical to the amino acid sequence shown in SEQ ID NO: 415.

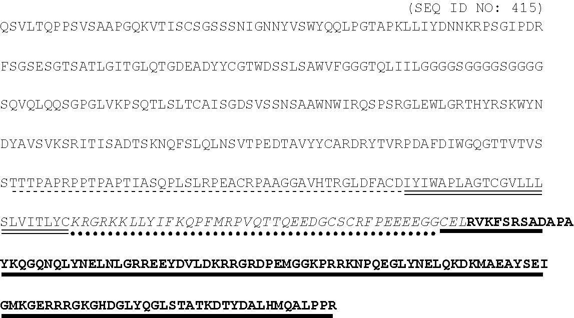

In some embodiments, a CAR construct as shown in SEQ ID NO: 415 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 41. CAR Clone 41

An exemplary CAR construct, as described herein, comprises a CLL-1 binding domain scFv comprising SEQ ID NO: 351, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 416, or an amino acid sequence that is at least 70%, at least 75%, at least 80%, at least 85%, at least 90%, at least 95%, or at least 99% identical to the amino acid sequence shown in SEQ ID NO: 416.

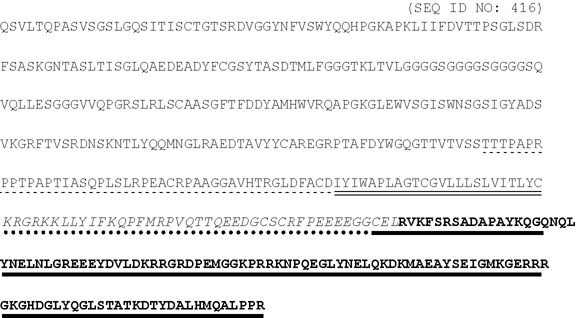

In some embodiments, a CAR construct as shown in SEQ ID NO: 416 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 42. CAR Clone 42

An exemplary CAR construct, as described herein, comprises a CLL-1 binding domain scFv comprising SEQ ID NO: 365, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 417, or an amino acid sequence that is at least 70%, at least 75%, at least 80%, at least 85%, at least 90%, at least 95%, or at least 99% identical to the amino acid sequence shown in SEQ ID NO: 417.

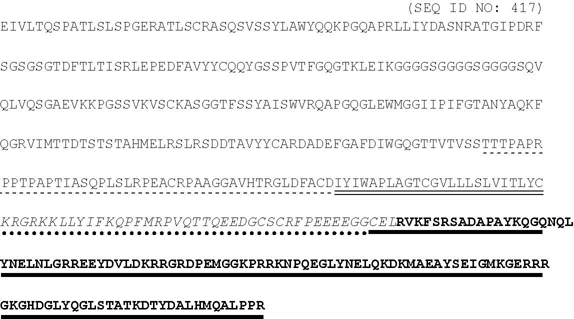

In some embodiments, a CAR construct as shown in SEQ ID NO: 417 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

43. CAR Clone 103.28z (Control)

An exemplary CAR construct, as described herein, comprises a CLL-1 binding domain scFv comprising SEQ ID NO: 922, a CD28 transmembrane domain, a CD28 hinge domain, a CD137 (4-1BB) co-stimulatory domain, and a CD3ζ intracellular signaling domain.

In some embodiments, a CAR comprises an amino acid sequence shown in SEQ ID NO: 922, or an amino acid sequence that is at least 70%, at least 75%, at least 80%, at least 85%, at least 90%, at least 95%, or at least 99% identical to the amino acid sequence shown in SEQ ID NO: 922.

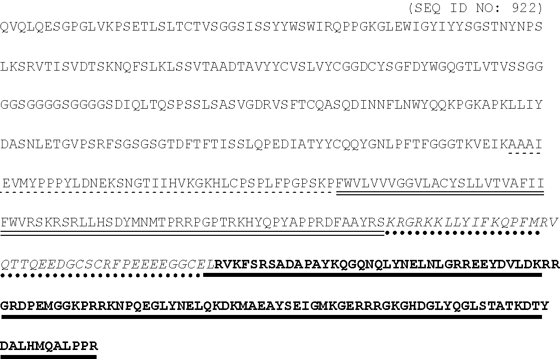

In some embodiments, a CAR construct as shown in SEQ ID NO: 922 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 44. CAR Clone 103.bbz (Control)

An exemplary CAR construct, as described herein, comprises a CLL-1 binding domain scFv comprising SEQ ID NO: 923, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 923, or an amino acid sequence that is at least 70%, at least 75%, at least 80%, at least 85%, at least 90%, at least 95%, or at least 99% identical to the amino acid sequence shown in SEQ ID NO: 923.

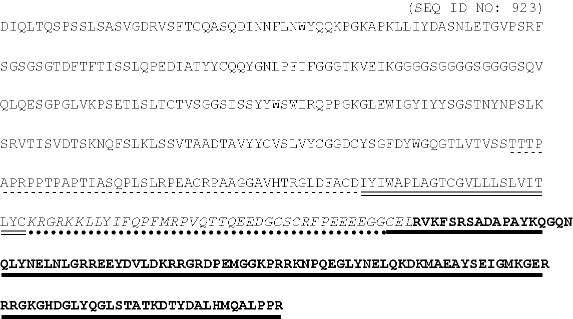

In some embodiments, a CAR construct as shown in SEQ ID NO: 923 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

45. CAR Clone h104

An exemplary CAR construct, as described herein, comprises a CLL-1 binding domain scFv comprising SEQ ID NO: 924, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 924, or an amino acid sequence that is at least 70%, at least 75%, at least 80%, at least 85%, at least 90%, at least 95%, or at least 99% identical to the amino acid sequence shown in SEQ ID NO: 924.

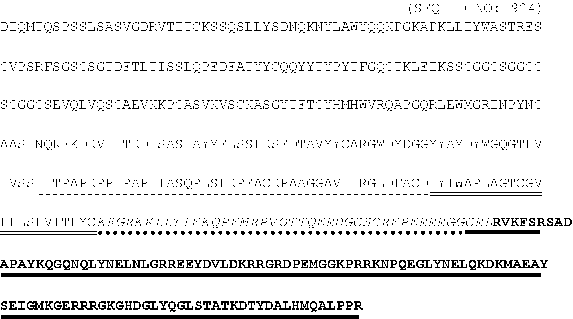

In some embodiments, a CAR construct as shown in SEQ ID NO: 924 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

46. CAR Clone m104

An exemplary CAR construct, as described herein, comprises a CLL-1 binding domain scFv comprising SEQ ID NO: 925, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 925, or an amino acid sequence that is at least 70%, at least 75%, at least 80%, at least 85%, at least 90%, at least 95%, or at least 99% identical to the amino acid sequence shown in SEQ ID NO: 925.

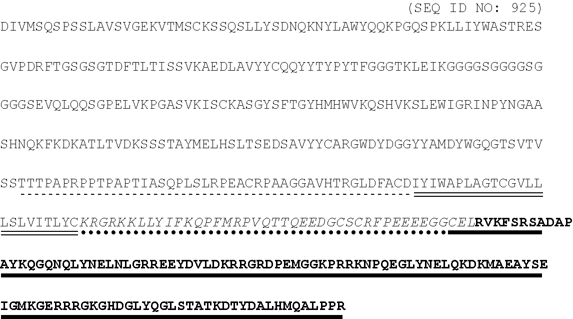

In some embodiments, a CAR construct as shown in SEQ ID NO: 925 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 47. CAR Clone 108

An exemplary CAR construct, as described herein, comprises a CLL-1 binding domain scFv comprising SEQ ID NO: 926, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 926, or an amino acid sequence that is at least 70%, at least 75%, at least 80%, at least 85%, at least 90%, at least 95%, or at least 99% identical to the amino acid sequence shown in SEQ ID NO: 926.

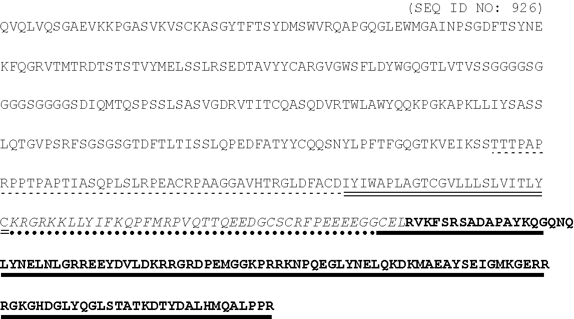

In some embodiments, a CAR construct as shown in SEQ ID NO: 926 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 48. CAR Clone 109

An exemplary CAR construct, as described herein, comprises a CLL-1 binding domain scFv comprising SEQ ID NO: 927, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 927, or an amino acid sequence that is at least 70%, at least 75%, at least 80%, at least 85%, at least 90%, at least 95%, or at least 99% identical to the amino acid sequence shown in SEQ ID NO: 927.

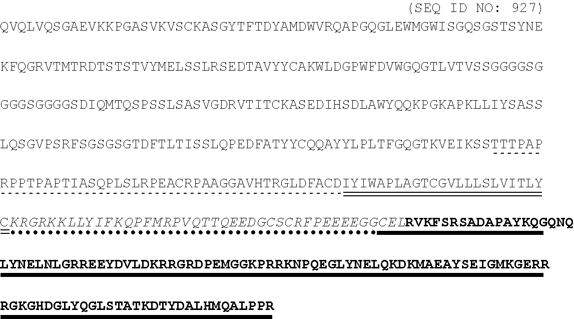

In some embodiments, a CAR construct as shown in SEQ ID NO: 927 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

### 49. CAR Clone 110

An exemplary CAR construct, as described herein, comprises a CLL-1 binding domain scFv comprising SEQ ID NO: 928, a CD8α transmembrane domain, a CD8α hinge domain, a CD137 (4-1BB) co-stimulatory domain, and a CD3ζ intracellular signaling domain.

In some embodiments, a CAR comprises an amino acid sequence shown in SEQ ID NO: 928, or an amino acid sequence that is at least 70%, at least 75%, at least 80%, at least 85%, at least 90%, at least 95%, or at least 99% identical to the amino acid sequence shown in SEQ ID NO: 928.

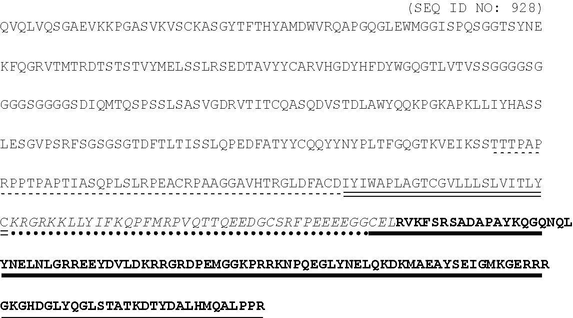

In some embodiments, a CAR construct as shown in SEQ ID NO: 928 is encoded in a recombinant expression vector. In some embodiments, the recombinant expression vector includes a promoter (e.g., an SFFV promoter, an EF1α promoter, a tEF1a promoter, a hPGK promoter, a SFFV promoter, or a MND promoter).

Any of the fusion proteins, such as any of the CARs described herein may be expressed in a cell and thereby presented on the surface of the cell. In some embodiments, the cell may be an immune cell, such as a T cell (i.e., T lymphocyte) or an NK cell. A T cell lymphocyte can be any T cell, such as a cultured T cell, e.g., a primary T cell, or a T cell from a cultured T cell line, e.g., TIB-153™, Jurkat, SupTl, etc., or a T cell obtained from a mammal (e.g., primary T cells).

### Nucleotide Sequences and Expression

The present disclosure includes nucleotide sequences encoding any one or more anti-CLL-1 antibodies described herein (e.g., a VHH described herein), or portion thereof (e.g., one or more CDRs described herein), and/or one or more fusion proteins described herein. In various instances, such nucleotide sequences may be present in a vector, such as an expression vector. In various instances such nucleotides may be present in the genome of a cell, e.g., a cell of a subject in need of treatment or a cell for production of an antibody, e.g., a mammalian cell for production of an antibody.

In some embodiments, any of the antibodies described herein are encoded by a polynucleotide comprised in a vector, e.g., a viral vector. Optionally, a polynucleotide encoding a polypeptide as described herein can be codon-optimized to enhance expression or stability. Codon optimization may be performed according to any standard methods known in the art. In some embodiments, expression of the polypeptide can be driven by a constitutively expressed promoter or an inducibly expressed promoter. In some embodiments, an antibody as described herein includes a signal peptide. Signal peptides can be derived from any protein that has an extracellular domain or is secreted. An antibody as described herein may include any signal peptides known in the art.

Retroviruses, such as lentiviruses, provide a convenient platform for delivery of nucleic acid sequences encoding a gene, or chimeric gene of interest. A selected nucleic acid sequence can be inserted into a vector and packaged in retroviral particles using techniques known in the art. The recombinant virus can then be isolated and delivered to cells, e.g., in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Calster Academic Press (ISBN:978-1-90455-55-4); and Hu and Pathak Pharmacological Reviews (2000) 52:493-512; which are incorporated by reference herein in their entirety. In some embodiments, an antibody described herein is expressed in a mammalian cell via transfection or electroporation of an expression vector comprising nucleic acid encoding the antibody. Transfection or electroporation methods are known in the art.

In another aspect, the disclosure relates to a cell, e.g., a mammalian cell, comprising any of the antibodies described herein; or a nucleic acid encoding any of the antibodies described herein. In one embodiment, the cell comprises an antibody described herein, or a nucleic acid encoding such an antibody described herein. The cell or tissue, e.g., mammalian cell or tissue, can be of human, primate, hamster, rabbit, rodent, cow, pig, sheep, horse, goat, dog or cat origin. In some embodiments, any other mammalian cell may be used. In some embodiments, the mammalian cell is human.

Efficient expression of an antibody described herein can be assessed using standard assays that detect the mRNA, DNA, or a gene product of the nucleic acid encoding the antibody, such as RT-PCR, FACS, Northern blotting, Western blotting, ELISA, or immunohistochemistry. In some embodiments, the antibody described herein is encoded by recombinant nucleic acid sequence.

CLL-1-Associated Diseases and/or Disorders

The present disclosure provides, among other things, compositions and methods for treating a disease associated with expression of CLL-1 or a condition associated with cells expressing CLL-1, including, e.g., a proliferative disease such as a cancer or malignancy (e.g., a hematopoietic malignancy), or a precancerous condition such as a myelodysplasia, a myelodysplastic syndrome or a preleukemia.

CLL-1, also known as CLEC12A, CD371, MICL, and CLL1, is a C-type lectin domain family 12 member A protein. CLL-1 is expressed on most granulocytes and monocytes but not on T, B, NK cells, and erythrocytes, and is thought to function in as a negative regulator of granulocyte and monocyte function. See, e.g., Wang et al. J. Hematol. Oncol. (2018) 11:7.

In some embodiments, the hematopoietic malignancy or hematological disorder is associated with CLL-1 expression. A hematopoietic malignancy has been described as a malignant abnormality involving hematopoietic cells (e.g., blood cells, including progenitor and stem cells). Examples of hematopoietic malignancies include, without limitation, Hodgkin lymphoma, non-Hodgkin lymphoma, leukemia, or multiple myeloma. Exemplary leukemias include, without limitation, acute myeloid leukemia, acute lymphoid leukemia, chronic myelogenous leukemia, acute lymphoblastic leukemia or chronic lymphoblastic leukemia, and chronic lymphoid leukemia.

In some embodiments, cells involved in the hematopoietic malignancy are resistant to conventional or standard therapeutics used to treat the malignancy. For example, the cells (e.g., cancer cells) may be resistant to a chemotherapeutic agent and/or CAR T cells used to treat the malignancy.

In some embodiments, the leukemia is acute myeloid leukemia (AML). Acute myeloid leukemia (AML) is a cancer of the bone marrow that needs more effective therapies. According to the National Cancer Institute, more than 60,000 people in the U.S. have AML, and less than 30% of patients survive five years following diagnosis. AML cells can be characterized and distinguished from other cells by detecting cell surface marker expression. AML cells can be CLL-1+, CD33+(though some are CD33−), CD45+, and CDw52+. AML is characterized as a heterogeneous, clonal, neoplastic disease that originates from transformed cells that have progressively acquired critical genetic changes that disrupt key differentiation and growth-regulatory pathways. See, e.g., Dohner et al., NEJM, (2015) 373:1136. Without wishing to be bound by theory, it is believed in some embodiments, that CLL-1 is expressed on myeloid leukemia cells as well as on normal myeloid and monocytic precursors and is an attractive target for AML therapy.

In some embodiments, the hematopoietic malignancy or hematological disease/disorder associated with CLL-1 is a precancerous condition such as a myelodysplasia, a myelodysplastic syndrome or a preleukemia. Myelodysplastic syndromes (MDS) are hematological medical conditions characterized by disorderly and ineffective hematopoiesis, or blood production. Thus, the number and quality of blood-forming cells decline irreversibly. Some patients with MDS can develop severe anemia, while others are asymptomatic. The classification scheme for MDS is known in the art, with criteria designating the ratio or frequency of particular blood cell types, e.g., myeloblasts, monocytes, and red cell precursors. MDS includes refractory anemia, refractory anemia with ring sideroblasts, refractory anemia with excess blasts, refractory anemia with excess blasts in transformation, chronic myelomonocytic leukemia (CML). In some embodiments, MDS can progress to AML.

In various instances, an antibody and/or fusion protein and/or cells expressing any of the foregoing described herein treats, alleviates, reduces the prevalence of, reduces the frequency of, or reduces the level or amount of one or more symptoms or biomarkers of a CLL-1-associated disorder (e.g., AML, MDS). Specific symptoms and progression of symptoms vary among subjects. Thus, in some embodiments, an antibody and/or fusion protein described herein is administered to a subject in need thereof, e.g., a subject having a CLL-1-associated disorder (e.g., AML, MDS). In some embodiments, administration of any of the antibodies and/or fusion proteins described herein prevents cancer or hematopoietic malignancy or pre-malignancy, including reducing one or more symptoms and/or delaying the progression of the disease.

In some cases, a subject may initially respond to a therapy (e.g., for a hematopoietic malignancy) and subsequently experience relapse. In some embodiments, the subject has or is susceptible to relapse of a hematopoietic malignancy (e.g., AML) following administration of one or more previous therapies. In some embodiments, the administration of any of the antibodies and/or fusion proteins described herein reduce the subject's risk of relapse or the severity of relapse.

In some embodiments, one or more anti-CLL-1 antibodies described herein are used in a method of treating one or more disorders described herein, e.g., one or more diseases or disorders associated with CLL-1 expression. In some embodiments, the method comprises administering to a subject in need thereof a therapeutically effective amount of an antibody, or antigen-binding fragment thereof, described herein, or a cell expressing any of the foregoing, or an antibody drug conjugate described herein. In some embodiments, one or more anti-CLL-1 antibodies described herein, including fusion proteins comprising any of the anti-CLL-1 antibodies, are used in a method of treating diseases or disorders associated with CLL-1 expression. In some embodiments, one or more anti-CLL-1 antibodies described herein are used in a method of treating neoplastic diseases and malignancies of the blood that are associated with CLL-1 expression. In some embodiments, one or more anti-CLL-1 antibodies described herein are used in a method of treating MDS or AML.

In various instances, administration of an antibody and/or fusion protein described herein results in a decrease in the prevalence, frequency, level, and/or amount of one or more symptoms or biomarkers of a CLL-1-associated disorder, e.g., a decrease of at least 3%, 4%, 5%, 6%, 7%, 8%, 9%, 10%, 15%, 20%, 25%, 30%, 35%, 40%, 45%, 50%, 60%, 70%, 80%, 90%, 95%, 99%, or 100% of one or more symptoms or biomarkers as compared to a prior measurement in the subject or to a reference value.

In some embodiments, an antibody and/or fusion protein described herein can be used in a number of diagnostic and/or therapeutic applications. For example, detectably-labeled versions of antibodies as described herein can be used in assays to detect the presence or amount of CLL-1 in a sample (e.g., a biological sample). Antibodies and/or fusion proteins described herein can be used in in vitro assays for studying inhibition of CLL-1 activity. In some embodiments, an antibody and/or fusion protein described herein can be used as a positive control in an assay designed to identify additional novel compounds that inhibit CLL-1 or otherwise are useful for treating a CLL-1-associated disorder.

Antibodies and/or fusion proteins described herein may be used in monitoring a subject, e.g., a subject having, suspected of having, at risk of developing, or under treatment for one or more CLL-1-associated disorders. Monitoring may include determining the amount or activity of CLL-1 in a subject, e.g., in the serum of a subject. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality, e.g., adding or dropping any of the treatments for a CLL-1-associated disorder described herein.

### Measuring Interactions of Antibodies and CLL-1

The binding properties of an antibody described herein to CLL-1 can be measured by methods known in the art, e.g., one of the following methods: BIACORE™ analysis, enzyme-linked immunosorbent assay (ELISA), x-ray crystallography, sequence analysis and scanning mutagenesis. The binding interaction of an antibody and CLL-1 can be analyzed using surface plasmon resonance (SPR). SPR or Biomolecular Interaction Analysis (BIA) detects bio-specific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden). Additionally, a KinExA® (Kinetic Exclusion Assay) assay, available from Sapidyne Instruments (Boise, Id.) can also be used.

Information from SPR can be used to provide an accurate and quantitative measure of the equilibrium dissociation constant (K_{D}), and kinetic parameters, including Kₒₙ and K_{off}, for the binding of an antibody to CLL-1. Such data can be used to compare different molecules. Information from SPR can also be used to develop structure-activity relationships (SAR). Variant amino acids at given positions can be identified that correlate with particular binding parameters, e.g., high affinity.

In certain embodiments, an antibody described herein exhibits high affinity for binding CLL-1. In various embodiments, K_{D} of an antibody as described herein for CLL-1 is less than about 10⁻⁴, 10⁻⁵, 10⁻⁶, 10⁻⁷, 10⁻⁸, 10⁻⁹, 10⁻¹⁰, 10¹¹, 10⁻¹², 10⁻¹³, 10⁻¹⁴, or 10⁻¹⁵ M. In certain instances, K_{D} of an antibody as described herein for CLL-1 is between 0.001 and 1 nM, e.g., 0.001 nM, 0.005 nM, 0.01 nM, 0.05 nM, 0.1 nM, 0.5 nM, or 1 nM.

In some embodiments, an antibody described herein binds to a specific epitope of CLL-1, e.g., comprising one or more specific amino acids of CLL-1. Without wishing to be bound by theory, disruption of the availability of the specific amino acids of a CLL-1 epitope of an antibody described herein (e.g., by mutagenesis or due to binding by a competing antibody) may decrease or eliminate binding of the antibody. In therapeutic applications or product manufacture where multiple anti-CLL-1 antibody to CLL-1 interactions are desired, it may be important to select or design anti-CLL-1 antibodies to target non-competing CLL-1 epitopes to avoid interfering with one another.

### Combination Therapy

In some embodiments, an anti-CLL-1 antibody described herein, or portion thereof (e.g., one or more CDRs described herein), and/or one or more fusion proteins described herein is administered in combination with one or more additional therapeutic agents, such as a chemotherapeutic agent or an oncolytic therapeutic agent. “Combination therapy,” as used herein, refers to those situations in which two or more different pharmaceutical agents are administered in overlapping regimens so that the subject is simultaneously exposed to both agents. When used in combination therapy, two or more different agents may be administered simultaneously or separately. Administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

As used herein, the term “chemotherapeutic agent” or “oncolytic therapeutic agent” (e.g., anti-cancer drug, e.g., anti-cancer therapy, e.g., immune cell therapy) has its art-understood meaning referring to one or more pro-apoptotic, cytostatic and/or cytotoxic agents, and/or hormonal agents, for example, specifically including agents utilized and/or recommended for use in treating one or more diseases, disorders or conditions associated with undesirable cell proliferation. In some embodiments, a chemotherapeutic agent and/or oncolytic therapeutic agent may be or comprise platinum compounds (e.g., cisplatin, carboplatin, and oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, and bendamustine), antitumor antibiotics (e.g., daunorubicin, doxorubicin, idarubicin, epirubicin, mitoxantrone, bleomycin, mytomycin C, plicamycin, and dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, and nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vicristine, vindesine, vinorelbine, and vinblastine), tyrosine kinase inhibitors (e.g., imatinib, dasatinib, nilotinib, sorafenib, and sunitinib), nitrosoureas (e.g., carmustine, fotemustine, and lomustine), hexamethylmelamine, mitotane, angiogenesis inhibitors (e.g., thalidomide and lenalidomide), steroids (e.g., prednisone, dexamethasone, and prednisolone), hormonal agents (e.g., tamoxifen, raloxifene, leuprolide, bicaluatmide, granisetron, and flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, and oxaprozin), selective cyclooxygenase-2 (COX-2) inhibitors, or any combination thereof.

In certain embodiments, chemotherapeutic agents and/or oncolytic therapeutic agents for anti-cancer treatment comprise biological agents such as tumor-infiltrating lymphocytes, CAR T-cells, antibodies, antigens, therapeutic vaccines (e.g., made from a patient's own tumor cells or other substances such as antigens that are produced by certain tumors), immune-modulating agents (e.g., cytokines, e.g., immunomodulatory drugs or biological response modifiers), checkpoint inhibitors or other immunologic agents. In certain embodiments, immunologic agents include immunoglobins, immunostimulants (e.g., bacterial vaccines, colony stimulating factors, interferons, interleukins, therapeutic vaccines, vaccine combinations, viral vaccines) and/or immunosuppressive agents (e.g., calcineurin inhibitors, interleukin inhibitors, TNF alpha inhibitors). In certain embodiments, hormonal agents include agents for anti-androgen therapy (e.g., Ketoconazole, ABiraterone, TAK-700, TOK-OOl, Bicalutamide, Nilutamide, Flutamide, Enzalutamide, ARN-509).

Additional chemotherapeutic agents and/or oncolytic therapeutic agents include immune checkpoint therapeutics (e.g., pembrolizumab, nivolumab, ipilimumab, atezolizumab, avelumab, durvalumab, tremelimumab, or cemiplimab), other monoclonal antibodies (e.g., rituximab, cetuximab, panetumumab, tositumomab, trastuzumab, alemtuzumab, gemtuzumab ozogamicin, bevacizumab, catumaxomab, denosumab, obinutuzumab, ofatumumab, ramucirumab, pertuzumab, nimotuzumab, lambrolizumab, pidilizumab, siltuximab, BMS-936559, RG7446/MPDL3280A, MEDI4736), antibody-drug conjugates (e.g., brentuximab vedotin (ADCETRIS®, Seattle Genetics); ado-trastuzumab emtansine (KADCYLA®, Roche); Gemtuzumab ozogamicin (Wyeth); CMC-544; SAR3419; CDX-011; PSMA-ADC; BT-062; and IMGN901 (see, e.g., Sassoon et al., Methods Mol. Biol. (2013) 1045:1-27; Bouchard et al., Bioorganic Med. Chem. Lett. (2014) 24: 5357-5363), or any combination thereof.

In some embodiments, combined administration of an anti-CLL-1 antibody or portion thereof (e.g., one or more CDRs described herein), and/or one or more fusion proteins described herein and an additional therapeutic agent result in an improvement in cancer to an extent that is greater than one produced by either the anti-CLL-1 antibody or the additional therapeutic agent alone. The difference between the combined effect and the effect of each agent alone can be a statistically significant difference. In some embodiments, the combined effect can be a synergistic effect. In some embodiments, combined administration of an anti-CLL-1 antibody or portion thereof (e.g., one or more CDRs described herein), and/or one or more fusion proteins described herein and an additional therapeutic agent allows administration of the additional therapeutic agent at a reduced dose, at a reduced number of doses, and/or at a reduced frequency of dosage compared to a standard dosing regimen, e.g., an approved dosing regimen for the additional therapeutic agent.

In some embodiments, treatment methods described herein are performed on subjects for whom other treatments of the medical condition have failed or have had less success in treatment through other means. Additionally, the treatment methods described herein can be performed in conjunction with one or more additional treatments of the medical condition. For instance, the method can comprise administering a cancer regimen, e.g., non-myeloablative chemotherapy, surgery, hormone therapy, and/or radiation, prior to, substantially simultaneously with, or after the administration of an anti-CLL-1 antibody described herein or portion thereof (e.g., one or more CDRs described herein), and/or one or more fusion proteins described herein, or composition thereof.

Aspects of the present disclosure involve administration of hematopoietic cells that are genetically modified to have reduced or eliminated expression of CLL-1, e.g., in the context of treating a subject in need of such hematopoietic stem cells, which may include, for example, a subject having a hematologic malignancy, such as, e.g., AML, or premalignancy, such as, e.g., MDS, and undergoing an immunotherapy regimen targeting CLL-1, e.g., a CLL-1-antibody-drug conjugate or a CLL-1 CAR-T or CAR-NK therapy. Such treatment regimen can involve, for example, the following steps: (1) administering a therapeutically effective amount of any of the anti-CLL-1 antibodies, CLL-1 binding fragments thereof, including fusion proteins, such as e.g., CARs, and cells expressing any of the foregoing, e.g., CAR-T or CAR-NK cells, as described herein or otherwise apparent to the skilled artisan based on the present disclosure; and (2) administering (e.g., infusing or reinfusing) the patient with hematopoietic stem cells, either autologous or allogeneic, where the hematopoietic cells have reduced expression or eliminated expression of CLL-1. In some embodiments, the hematopoietic cells are genetically modified to have reduced expression of CLL-1. In some embodiments, the hematopoietic cells are genetically modified to have eliminated expression of CLL-1. In some embodiments, the hematopoietic cells are genetically modified to have reduced or eliminated expression of a CLL-1 epitope bound by an antibody, a CLL-1-binding fragment thereof, or portion thereof (e.g., one or more CDRs described herein), and/or one or more fusion proteins described herein.

### Formulations and Administration

In various embodiments, any of the antibodies or portion thereof (e.g., one or more CDRs described herein), and/or one or more fusion proteins described herein can be incorporated into a pharmaceutical composition. Such a pharmaceutical composition can be useful, e.g., for the prevention and/or treatment of diseases, e.g., cancers, such as AML, MDS. Pharmaceutical compositions can be formulated by methods known to those skilled in the art (such as described in Remington's Pharmaceutical Sciences, 17th edition, ed. Alfonso R. Gennaro, Mack Publishing Company, Easton, Pa. (1985)).

In some embodiments, a pharmaceutical composition can be formulated to include a pharmaceutically acceptable carrier or excipient. Examples of pharmaceutically acceptable carriers include, without limitation, any and all solvents, dispersion media, coatings, antibacterial and antifungal agents, isotonic and absorption delaying agents, and the like that are physiologically compatible. Compositions of the present invention can include a pharmaceutically acceptable salt, e.g., an acid addition salt or a base addition salt.

In some embodiments, a composition including an antibody as described herein, e.g., a sterile formulation for injection, can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, such as, for example, an alcohol such as ethanol and/or a polyalcohol such as propylene glycol or polyethylene glycol, and/or a nonionic surfactant such as polysorbate 80™ or HCO-50.

As disclosed herein, a pharmaceutical composition may be in any form known in the art. Such forms include, e.g., liquid, semi-solid and solid dosage forms, such as liquid solutions (e.g., injectable and infusible solutions), dispersions or suspensions, tablets, pills, powders, liposomes and suppositories.

Selection or use of any particular form may depend, in part, on the intended mode of administration and therapeutic application. For example, compositions containing a composition intended for systemic or local delivery can be in the form of injectable or infusible solutions. Accordingly, compositions can be formulated for administration by a parenteral mode (e.g., intravenous, subcutaneous, intraperitoneal, or intramuscular injection). As used herein, parenteral administration refers to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

Route of administration can be parenteral, for example, administration by injection, transnasal administration, transpulmonary administration, or transcutaneous administration. Administration can be systemic or local by intravenous injection, intramuscular injection, intraperitoneal injection, or subcutaneous injection.

In some embodiments, a pharmaceutical composition of the present invention can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filter sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

A pharmaceutical composition can be administered parenterally in the form of an injectable formulation comprising a sterile solution or suspension in water or another pharmaceutically acceptable liquid. For example, the pharmaceutical composition can be formulated by suitably combining the therapeutic molecule with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of active ingredient included in a pharmaceutical preparation is such that a suitable dose within the designated range is provided. Non-limiting examples of oily liquid include sesame oil and soybean oil, and may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. A formulated injection can be packaged in a suitable ampule.

In various embodiments, subcutaneous administration can be accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for combining with antibody drug for subcutaneous injection.

An injection system of the present disclosure may employ a delivery pen as described in U.S. Pat. No. 5,308,341. Pen devices, most commonly used for self-delivery of insulin to patients with diabetes, are well known in the art. Such devices can comprise at least one injection needle (e.g., a 31 gauge needle of about 5 to 8 mm in length), are typically pre-filled with one or more therapeutic unit doses of a therapeutic solution, and are useful for rapidly delivering solution to a subject with as little pain as possible. One medication delivery pen includes a vial holder into which a vial of a therapeutic or other medication may be received. The pen may be an entirely mechanical device or it may be combined with electronic circuitry to accurately set and/or indicate the dosage of medication that is injected into the user. See, e.g., U.S. Pat. No. 6,192,891. In some embodiments, the needle of the pen device is disposable and the kits include one or more disposable replacement needles. Pen devices suitable for delivery of any one of the presently featured compositions are also described in, e.g., U.S. Pat. Nos. 6,277,099; 6,200,296; and 6,146,361, the disclosures of each of which are incorporated herein by reference in their entirety. A microneedle-based pen device is described in, e.g., U.S. Pat. No. 7,556,615, the disclosure of which is incorporated herein by reference in its entirety. See also the Precision Pen Injector (PPI) device, MOLLY™, manufactured by Scandinavian Health Ltd.

In some embodiments, a composition described herein can be therapeutically delivered to a subject by way of local administration. As used herein, “local administration” or “local delivery,” can refer to delivery that does not rely upon transport of the composition or agent to its intended target tissue or site via the vascular system. For example, the composition may be delivered by injection or implantation of the composition or agent or by injection or implantation of a device containing the composition or agent. In certain embodiments, following local administration in the vicinity of a target tissue or site, the composition or agent, or one or more components thereof, may diffuse to an intended target tissue or site that is not the site of administration.

In some embodiments, a composition can be formulated for storage at a temperature below 0° C. (e.g., −20° C. or −80° C.). In some embodiments, the composition can be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, in some embodiments, the compositions described herein are stable in storage for at least 1 year at 2-8° C. (e.g., 4° C.).

In some embodiments, a pharmaceutical composition can be formulated as a solution. In some embodiments, a composition can be formulated, for example, as a buffered solution at a concentration suitable for storage at 2-8° C. (e.g., 4° C.).

Compositions including one or more antibodies as described herein can be formulated in immunoliposome compositions. Such formulations can be prepared by methods known in the art. Liposomes with enhanced circulation time are disclosed in, e.g., U.S. Pat. No. 5,013,556.

In certain embodiments, compositions can be formulated with a carrier that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art. See, e.g., J. R. Robinson (1978) “Sustained and Controlled Release Drug Delivery Systems,” Marcel Dekker, Inc., New York.

In some embodiments, administration of an antibody as described herein is achieved by administering to a subject a nucleic acid encoding the antibody. Nucleic acids encoding a therapeutic antibody described herein can be incorporated into a gene construct to be used as a part of a gene therapy protocol to deliver nucleic acids that can be used to express and produce antibody within cells. Expression constructs of such components may be administered in any therapeutically effective carrier, e.g., any formulation or composition capable of effectively delivering the component gene to cells in vivo. Approaches include insertion of the subject gene in viral vectors including recombinant retroviruses, adenovirus, adeno-associated virus, lentivirus, and herpes simplex virus-1 (HSV-1), or recombinant bacterial or eukaryotic plasmids. Viral vectors can transfect cells directly; plasmid DNA can be delivered with the help of, for example, cationic liposomes (lipofectin) or derivatized, polylysine conjugates, gramicidin S, artificial viral envelopes or other such intracellular carriers, as well as direct injection of the gene construct or CaPO₄ precipitation (see, e.g., WO04/060407). Examples of suitable retroviruses include pLJ, pZIP, pWE and pEM which are known to those skilled in the art (see, e.g., Eglitis et al. Science (19785) 230:1395-1398; Danos and Mulligan Proc Natl Acad Sci USA (1988) 85:6460-6464; Wilson et al. Proc Natl Acad Sci USA (1988) 85:3014-3018; Armentano et al. Proc Natl Acad Sci USA (1990) 87:6141-6145; Huber et al. Proc Natl Acad Sci USA (1991) 88:8039-8043; Ferry et al. Proc Natl Acad Sci USA (1991) 88:8377-8381; Chowdhury et al. Science (1991) 254:1802-1805; van Beusechem et al. Proc Natl Acad Sci USA (1992) 89:7640-7644; Kay et al. Human Gene Therapy (1992)3:641-647; Dai et al. Proc Natl Acad Sci USA (1992) 89:10892-10895; Hwu et al. J Immunol (1993) 150:4104-4115; U.S. Pat. Nos. 4,868,116 and 4,980,286; and PCT Publication Nos. WO89/07136, WO89/02468, WO89/05345, and WO92/07573). Another viral gene delivery system utilizes adenovirus-derived vectors (see, e.g., Berkner et al. BioTechniques (1988) 6:616; Rosenfeld et al. Science (1991) 252:431-434; and Rosenfeld et al. Cell (1992) 68:143-155). Suitable adenoviral vectors derived from the adenovirus strain Ad type 5 d1324 or other strains of adenovirus (e.g., Ad2, Ad3, Ad7, etc.) are known to those skilled in the art. Yet another viral vector system useful for delivery of the subject gene is the adeno-associated virus (AAV). See, e.g., Flotte et al. Am J Respir Cell Mol Biol (1992) 7:349-356; Samulski et al. J Virol (1989) 63:3822-3828; and McLaughlin et al. J Virol (1989) 62:1963-1973.

In some embodiments, the compositions provided herein are present in unit dosage form, which unit dosage form can be suitable for self-administration. Such a unit dosage form may be provided within a container, typically, for example, a vial, cartridge, prefilled syringe or disposable pen. A doser such as the doser device described, for example, in U.S. Pat. No. 6,302,855, may also be used, for example, with an injection system as described herein.

A suitable dose of a composition described herein, which dose is capable of treating or preventing a disorder in a subject, can depend on a variety of factors including, e.g., the age, sex, and weight of a subject to be treated and the particular inhibitor compound used. For example, a different dose of one composition including an antibody, or portion thereof (e.g., one or more CDRs described herein), and/or one or more fusion proteins described herein may be required to treat a subject with a cancer (e.g., AML) as compared to the dose of a different formulation of that antibody. Other factors affecting the dose administered to the subject include, e.g., the type or severity of the disorder. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject can also be adjusted based upon the judgment of the treating medical practitioner.

A pharmaceutical solution can include a therapeutically effective amount of a composition described herein. Such effective amounts can be readily determined by one of ordinary skill in the art based, in part, on the effect of the administered composition, or the combinatorial effect of the composition and one or more additional active agents, if more than one agent is used. A therapeutically effective amount of a composition described herein can also vary according to factors such as the disease state, age, sex, and weight of the individual, and the ability of the composition (and one or more additional active agents) to elicit a desired response in the individual, e.g., amelioration of at least one condition parameter, e.g., amelioration of at least one symptom of a cancer (e.g., AML). For example, a therapeutically effective amount of a composition described herein can inhibit (lessen the severity of or eliminate the occurrence of) and/or prevent a particular disorder, and/or any one of the symptoms of the particular disorder known in the art or described herein. A therapeutically effective amount is also one in which any toxic or detrimental effects of the composition are outweighed by the therapeutically beneficial effects.

Suitable human doses of any of the compositions described herein can further be evaluated in, e.g., Phase I dose escalation studies. See, e.g., van Gurp et al. Am J Transplantation (2008) 8(8):1711-1718; Hanouska et al. Clin Cancer Res (2007) 13(2, part 1):523-531; and Hetherington et al. Antimicrobial Agents and Chemotherapy (2006) 50(10): 3499-3500.

Toxicity and therapeutic efficacy of compositions can be determined by known pharmaceutical procedures in cell cultures or experimental animals (e.g., animal models of any of the cancers described herein). These procedures can be used, e.g., for determining the LD₅₀ (the dose lethal to 50% of the population) and the EDso (the dose therapeutically effective in 50% of the population). The dose ratio between toxic and therapeutic effects is the therapeutic index and it can be expressed as the ratio LD₅₀/ED₅₀. A composition described herein that exhibits a high therapeutic index is preferred. While compositions that exhibit toxic side effects may be used, care should be taken to design a delivery system that targets such compounds to the site of affected tissue and to minimize potential damage to normal cells and, thereby, reduce side effects. Those of skill in the art will appreciate that data obtained from cell culture assays and animal studies can be used in formulating a range of dosage for use in humans. Appropriate dosages of compositions described herein lie generally within a range of circulating concentrations of the compositions that include the ED₅₀ with little or no toxicity. The dosage may vary within this range depending upon the dosage form employed and the route of administration utilized. For a composition described herein, the therapeutically effective dose can be estimated initially from cell culture assays. A dose can be formulated in animal models to achieve a circulating plasma concentration range that includes the IC₅₀ (i.e., the concentration of the antibody which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration (e.g., to the eye or a joint) is desired, cell culture or animal modeling can be used to determine a dose required to achieve a therapeutically effective concentration within the local site.

### CAR and CAR-T Cell Characterization Assays

In various embodiments, one or more CAR characterization assays are used to assess the activity of the CARs and activation of a cell (e.g., T cells) expressing the CARs comprising any of the anti-CLL-1 antibodies described herein.

Some suitable CAR characterization assays are provided herein. Exemplary CAR characterization assays include but are not limited to cytotoxicity assays (e.g., chromium (⁵¹Cr)-release assay, luciferase-mediated bioluminescence imaging (BLI) assay, impedance-based assay, and flow cytometry assay), cytokine excretion assays to quantify various cytokines released by CAR-T cells using either flow cytometry-based methods or enzyme-linked immunosorbent assays (e.g. interleukin-σ secretion assay), or reporter constructs (e.g. IL-2 Reporter Systems (IRS)). Additional suitable CAR characterization assays will be apparent to those of skill in the art based on the present disclosure. Exemplary CAR characterization assays are described in, for example, MCB: CAR T Cells: Development, Characterization and Applications. United Kingdom: Elsevier Science, 2022; Cell Reprogramming for Immunotherapy, Springer Nature 2020, Vol. 2097; Zaritskaya et al., Expert Rev Vaccines. 2010 June; 9(6): 601-616; Xi et al., J Vis Exp. 2019; (153); Mukherjee et al., Mol Ther. 2017; 25(8):1757-1768, which are incorporated by reference herein in their entirety.

In some embodiments, an IL-2 reporter system comprising a minimal nuclear factor of activated T cells (NFAT)-responsive promoter (i.e., an NFAT-responsive reporter system) is used to assess the activity of CARs and activation of a cell (e.g., T cells) expressing the CARs comprising any of the anti-CLL-1 antibodies described herein. CAR activation sets in motion an intracellular pathway leading to T-cell activation and effector function of the T cell, which involves NFAT signaling and gene expression (see, e.g., Hogan, Cell Calcium. (2017) 63:66-9). As used herein, the term “NFAT-responsive promoter” refers to a promoter region that is activated by NFAT signaling and promotes expression of a gene that is operably linked to the NFAT-responsive promoter upon activation. In some embodiments, the gene that is operably linked (under control of) the NFAT-responsive promoter encodes a reporter molecule. Various NFAT-responsive reporter systems are described in U.S. Provisional Patent Application Nos. 63/227,046 and 63/278,613, which are incorporated by reference in their entirety.

### Hematopoietic Cells Deficient in a Lineage-Specific Cell-Surface Antigen

Additionally, the present disclosure provides methods for the combined inhibition of CLL-1 lineage specific antigen. Such treatment regimen can involve the following steps: (1) administering a therapeutically effective amount of an immune cell described herein (e.g., a T cell) to the patient, where the immune cell comprises a nucleic acid sequence encoding a chimeric antigen receptor (CAR) targeting CLL-1 lineage specific antigens; and (2) infusing or reinfusing the patient with hematopoietic stem cells, either autologous or allogeneic, where the hematopoietic cells have reduced expression of CLL-1 lineage specific antigens.

In some embodiments, the hematopoietic cells are hematopoietic stem cells. Hematopoietic stem cells (HSCs) are capable of giving rise to both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. HSCs are characterized by the expression of the cell surface marker CD34 (e.g., CD34+), which can be used for the identification and/or isolation of HSCs, and absence of cell surface markers associated with commitment to a cell lineage.

In some embodiments, HSCs are obtained from a subject, such as a mammalian subject. In some embodiments, the mammalian subject is a non-human primate, a rodent (e.g., mouse or rat), a bovine, a porcine, an equine, or a domestic animal. In some embodiments, HSCs are obtained from a human patient, such as a human patient having a hematopoietic malignancy. In some embodiments, HSCs are obtained from a healthy donor. In some embodiments, HSCs are obtained from the subject to whom the immune cells expressing the chimeric receptors will be subsequently administered.

HSCs may be obtained from any suitable source using convention means known in the art. In some embodiments, HSCs are obtained from a sample from a subject, such as bone marrow sample or from a blood sample. Alternatively, or in addition, HSCs may be obtained from an umbilical cord. In some embodiments, the HSCs are from bone marrow or peripheral blood mononuclear cells (PBMCs). In general, bone marrow cells may be obtained from iliac crest, femora, tibiae, spine, rib or other medullary spaces of a subject. Bone marrow may be taken out of the patient and isolated through various separations and washing procedures known in the art. An exemplary procedure for isolation of bone marrow cells comprises the following steps: a) extraction of a bone marrow sample; b) centrifugal separation of bone marrow suspension in three fractions and collecting the intermediate fraction, or buffycoat; c) the buffycoat fraction from step (b) is centrifuged one more time in a separation fluid, commonly Ficoll™, and an intermediate fraction which contains the bone marrow cells is collected; and d) washing of the collected fraction from step (c) for recovery of re-transfusable bone marrow cells.

HSCs typically reside in the bone marrow but can be mobilized into the circulating blood by administering a mobilizing agent in order to harvest HSCs from the peripheral blood. In some embodiments, the subject from which the HSCs are obtained is administered a mobilizing agent, such as granulocyte colony-stimulating factor (G-CSF). The number of the HSCs collected following mobilization using a mobilizing agent is typically greater than the number of cells obtained without use of a mobilizing agent.

In some embodiments, a sample is obtained from a subject and is then enriched for a desired cell type (e.g., CD34+/CLL-1− cells). For example, PBMCs and/or CD34+hematopoietic cells can be isolated from blood as described herein. Cells can also be isolated from other cells, for example by isolation and/or activation with an antibody binding to an epitope on the cell surface of the desired cell type. Another method that can be used includes negative selection using antibodies to cell surface markers to selectively enrich for a specific cell type without activating the cell by receptor engagement.

Populations of HSC can be expanded prior to or after genetically engineering the HSC to become deficient in a lineage specific cell-surface antigen. The cells may be cultured under conditions that comprise an expansion medium comprising one or more cytokines, such as stem cell factor (SCF), Flt-3 ligand (FLt3L), thrombopoietin (TPO), Interleukin 3 (IL-3), or Interleukin 6 (IL-6). The cell may be expanded for about 1, 2, 3, 4, 5, 6, 7, 8, 9, 10, 11, 12, 13, 14, 15, 16, 17, 18, 19, 20, 21, 22, 23, 23, 25 days or any range necessary. In some embodiments, HSCs are expanded after isolation of a desired cell population (e.g., CD34+/CLL-1−) from a sample obtained from a subject and prior to genetic engineering. In some embodiments, the HSC are expanded after genetic engineering, thereby selectively expanding cells that have undergone the genetic modification and are deficient in a lineage-specific cell-surface antigen. In some embodiments, a cell (“a clone”) or several cells having a desired characteristic (e.g., phenotype or genotype) following genetic modification may be selected and independently expanded.

In some embodiments, the hematopoietic cells are genetically engineered to be deficient in a cell-surface lineage-specific antigen. In some embodiments, the hematopoietic cells are genetically engineered to be deficient in the same cell-surface lineage-specific antigen that is targeted by the agent. As used herein, a hematopoietic cell is considered to be deficient in a cell-surface lineage-specific antigen if hematopoietic cell has substantially reduced expression of the cell-surface lineage-specific antigen as compared to a naturally occurring hematopoietic cell of the same type as the genetically engineered hematopoietic cell (e.g., is characterized by the presence of the same cell surface markers, such as CD34).

In some embodiments, the hematopoietic cell has no detectable expression of the cell-surface lineage-specific antigen. The expression level of a cell-surface lineage-specific antigen can be assessed by any means known in the art. For example, the expression level of a cell surface lineage-specific antigen can be assessed by detecting the antigen with an antigen specific antibody (e.g., flow cytometry methods, Western blotting).

In some embodiments, the expression of the cell-surface lineage-specific antigen on the genetically engineered hematopoietic cell is compared to the expression of the cell-surface lineage-specific antigen on a naturally occurring hematopoietic cell. In some embodiments, the genetic engineering results in a reduction in the expression level of the cell-surface lineage-specific antigen by at least about 50%, 60%, 70%, 80%, 90%, 91%, 92%, 93%, 94%, 95%, 96%, 97%, 98%, or 99% as compared to the expression of the cell-surface lineage-specific antigen on a naturally occurring hematopoietic cell. In some embodiments, the hematopoietic cell is deficient in the whole endogenous gene encoding the cell-surface lineage-specific antigen. In some embodiments, the whole endogenous gene encoding the cell-surface lineage-specific antigen has been deleted. In some embodiments, the hematopoietic cell comprises a portion of endogenous gene encoding the cell-surface lineage-specific antigen. In some embodiments, the hematopoietic cell expressing a portion (e.g., a truncated protein) of the cell-surface lineage specific antigen. In some embodiments, a portion of the endogenous gene encoding the cell surface lineage-specific antigen has been deleted. In some embodiments, at least 10%, 20%, 30%, 40%, 50%, 60%, 70% or more of the gene encoding the cell-surface lineage-specific antigen has been deleted.

As will be appreciated by one of ordinary skill in the art, a portion of the nucleotide sequence encoding the cell-surface lineage-specific antigen may be deleted or one or more non-coding sequences, such that the hematopoietic cell is deficient in the antigen (e.g., has substantially reduced expression of the antigen).

In some embodiments, the cell-surface lineage-specific antigen is CLL-1. In some embodiments, a portion of CLL-1 is deleted.

Any of the genetically engineering hematopoietic cells, such as HSCs, that are deficient in a cell-surface lineage-specific antigen can be prepared by a routine method or by a method described herein. In some embodiments, the genetic engineering is performed using genome editing. As used herein, “genome editing” refers to a method of modifying the genome, including any protein-coding or non-coding nucleotide sequence, of an organism to knock out the expression of a target gene. In general, genome editing methods involve use of an endonuclease that is capable of cleaving the nucleic acid of the genome, for example at a targeted nucleotide sequence. Repair of the double-stranded breaks in the genome may be repaired introducing mutations and/or exogenous nucleic acid may be inserted into the targeted site.

Genome editing methods are generally classified based on the type of endonuclease that is involved in generating double stranded breaks in the target nucleic acid. These methods include use of zinc finger nucleases (ZFN), transcription activator-like effector-based nuclease (TALEN), meganucleases, and CRISPR/Cas systems. Methods of editing the genome of HSCs described herein can be found, e.g., in International Publication Nos. WO 2017/066760, WO 2020/047164, WO 2021/041971, and WO 2022/047168, all of which are incorporated by reference in their entirety.

### Combination Therapy

As described herein, agents comprising an antigen-binding domain that binds to a cell-surface lineage-specific antigen (e.g., CLL-1 CAR) may be administered to a subject in combination with hematopoietic cells that are deficient for the cell-surface lineage-specific antigen.

In some embodiments, the agents and/or the hematopoietic cells may be mixed with a pharmaceutically acceptable carrier to form a pharmaceutical composition, which is also within the scope of the present disclosure.

To perform the methods described herein, an effective amount of the agent comprising an antigen-binding domain that binds to a cell-surface lineage-specific antigen and an effective amount of hematopoietic cells can be co-administered to a subject in need of. the treatment.

As described herein, the hematopoietic cells and/or immune cells expressing chimeric receptors may be autologous to the subject. i.e., the cells are obtained from the subject in need of the treatment, genetically engineered to be deficient for expression of the cell-surface lineage-specific antigen or for expression of the chimeric receptor constructs, and then administered to the same subject. Administration of autologous cells to a subject may result in reduced rejection of the host cells as compared to administration of non-autologous cells. Alternatively, the host cells are allogeneic cells, i.e., the cells are obtained from a first subject, genetically engineered to be deficient for expression of the cell-surface lineage-specific antigen or for expression of the chimeric receptor constructs, and administered to a second subject that is different from the first subject but of the same species. For example, allogeneic immune cells may be derived from a human donor and administered to a human recipient who is different from the donor.

In some embodiments, the immune cells and/or hematopoietic cells are allogeneic cells and have been further genetically engineered to reduced graft-versus-host disease. For example, as described herein, the hematopoietic stem cells may be genetically engineered (e.g., using genome editing) to have reduced expression of CD45RA.

In some embodiments, the immune cells expressing any of the chimeric receptors described herein are administered to a subject in an amount effective in to reduce the number of target cells (e.g., cancer cells) by least 20%, e.g., 50%, 80%, 100%, 2-fold, 5-fold, 10-fold, 20-fold, 50-fold, 100-fold or more.

A typical amount of cells, i.e., immune cells or hematopoietic cells, administered to a mammal (e.g., a human) can be, for example, in the range of one million to 100 billion cells; however, amounts below or above this exemplary range are also within the scope of the present disclosure. For example, the daily dose of cells can be about 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), preferably about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), more preferably about 100 million cells to about 50 billion cells (e.g., about 120 million cells, about 350 million cells, about 350 million cells, about 450 million cells, about 650 million cells, about 800 million cells, about 900 million cells, about 3 billion cells, about 30 billion cells, about 45 billion cells, or a range defined by any two of the foregoing values).

In some embodiments, the chimeric receptor (e.g., a nucleic acid encoding the chimeric receptor) is introduced into an immune cell, and the subject (e.g., human patient) receives an initial administration or dose of the immune cells expressing the chimeric receptor. One or more subsequent administrations of the agent (e.g., immune cells expressing the chimeric receptor) may be provided to the patient at intervals of 15 days, 14, 13, 12, 11, 10, 9, 8, 7, 6, 5, 4, 3, or 2 days after the previous administration. More than one dose of the agent can be administered to the subject per week, e.g., 2, 3, 4, or more administrations of the agent. The subject may receive more than one doses of the agent (e.g., an immune cell expressing a chimeric receptor) per week, followed by a week of no administration of the agent, and finally followed by one or more additional doses of the agent (e.g., more than one administration of immune cells expressing a chimeric receptor per week). The immune cells expressing a chimeric receptor may be administered every other day for 3 administrations per week for two, three, four, five, six, seven, eight or more weeks.

In some embodiments, an agent comprises an antigen-binding domain that binds a cell-surface lineage-specific antigen and a population of hematopoietic cells deficient in the cell-surface lineage-specific antigen. Accordingly, in such therapeutic methods, the agent recognizes (binds) a target cell expressing the cell-surface lineage-specific antigen for targeting killing. The hematopoietic cells that are deficient in the antigen allow for repopulation of a cell type that is targeted by the agent. In some embodiments, the treatment of the patient can involve the following steps: (1) administering a therapeutically effective amount of an agent targeting a cell-surface lineage-specific antigen to the patient and (2) infusing or reinfusing the patient with hematopoietic stem cells, either autologous or allogenic, where the hematopoietic cells have reduced expression of a lineage specific disease-associated antigen. In some embodiments, the treatment of the patient can involve the following steps: (1) administering a therapeutically effective amount of an immune cell expressing a chimeric receptor to the patient, wherein the immune cell comprises a nucleic acid sequence encoding a chimeric receptor that binds a cell-surface lineage-specific, disease associated antigen; and (2) infusing or reinfusing the patient with hematopoietic cells (e.g., hematopoietic stem cells), either autologous or allogenic, where the hematopoietic cells have reduced expression of a lineage specific disease-associated antigen.

The efficacy of the therapeutic methods using an agent comprising an antigen-binding fragment that binds a cell-surface lineage-specific antigen and a population of hematopoietic cells deficient in the cell-surface lineage-specific antigen may be assessed by any method known in the art and would be evident to a skilled medical professional. For example, the efficacy of the therapy may be assessed by survival of the subject or cancer burden in the subject or tissue or sample thereof. In some embodiments, the efficacy of the therapy is assessed by quantifying the number of cells belonging to a particular population or lineage of cells. In some embodiments, the efficacy of the therapy is assessed by quantifying the number of cells presenting the cell-surface lineage-specific antigen. In some embodiments, the agent comprising an antigen-binding fragment that binds to the cell-surface lineage-specific antigen and the population of hematopoietic cells is administered concomitantly.

In some embodiments, the agent comprising an antigen-binding fragment that binds a cell-surface lineage-specific antigen (e.g., immune cells expressing a chimeric receptor as described herein) is administered prior to administration of the hematopoietic cells. In some embodiments, the agent comprising an antigen-binding fragment that binds a cell-surface lineage-specific antigen (e.g., immune cells expressing a chimeric receptor as described herein) is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the hematopoietic cells.

In some embodiments, the hematopoietic cells are administered prior to the agent comprising an antigen-binding fragment that binds a cell-surface lineage-specific antigen (e.g., immune cells expressing a chimeric receptor as described herein). In some embodiments, the population of hematopoietic cells is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the agent comprising an antigen-binding fragment that binds to the cell-surface lineage-specific antigen.

In some embodiments, the agent targeting the cell-surface lineage-specific antigen and the population of hematopoietic cells are administered at substantially the same time. In some embodiments, agent targeting the cell-surface lineage-specific antigen is administered and the patient is assessed for a period of time, the population of hematopoietic cells is administered, and the patient is assessed for a period of time, after which agent targeting the cell-surface lineage-specific antigen is administered.

Also within the scope of the present disclosure are multiple administrations (e.g., doses) of the agents and/or populations of hematopoietic cells. In some embodiments, the agents and/or populations of hematopoietic cells are administered to the subject once. In some embodiments, agents and/or populations of hematopoietic cells are administered to the subject more than once (e.g., at least 2, 3, 4, 5, or more times). In some embodiments, the agents and/or populations of hematopoietic cells are administered to the subject at a regular interval, e.g., every six months.

In some embodiments, the subject is a human subject having a hematopoietic malignancy. As used herein a hematopoietic malignancy refers to a malignant abnormality involving hematopoietic cells (e.g., blood cells, including progenitor and stem cells). Examples of hematopoietic malignancies include, without limitation, Hodgkin's lymphoma, non-Hodgkin's lymphoma, leukemia, or multiple myeloma. Leukemias include acute myeloid leukemia, chronic myelogenous leukemia, chronic lymphoblastic leukemia, and chronic lymphoid leukemia.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.): Handbook of Experimental Immunology (D. M. Weir and C. C. Blackwell, eds.): Gene Transfer Vectors for Mammalian Cells (J. M. Miller and M. P. Calos, eds., 1987; Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985; Transcription and Translation (B. D. Hames & S. J. Higgins, eds. (1984»; Animal Cell Culture (R. I. Freshney, ed. (1986»; Immobilized Cells and Enzymes (IRL Press, (1986; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXAMPLES

### Example 1. Generation of Novel Antibodies Against CLL-1

A fully human scFv phage display library and a fully human VHH (heavy chain only) phage display library were panned and screened to identify single domain antibodies that specifically recognize CLL-1. A recombinant CLL-1 protein was used during panning. A HL-60 cell line and a U937 cell line were used as CLL-1-positive cell lines, and a HL-60 cell line harboring a knockout of CLL-1 was used as a CLL-1 negative cell line. A purified anti-human CLL-1 antibody was used as a positive control. Specific binding activity of identified binders was confirmed by FACS flow cytometry as described below. The heavy chain variable region sequences of the identified CLL-1 binders are included herein.

### Flow Cytometry Analysis of CLL-1 Binders

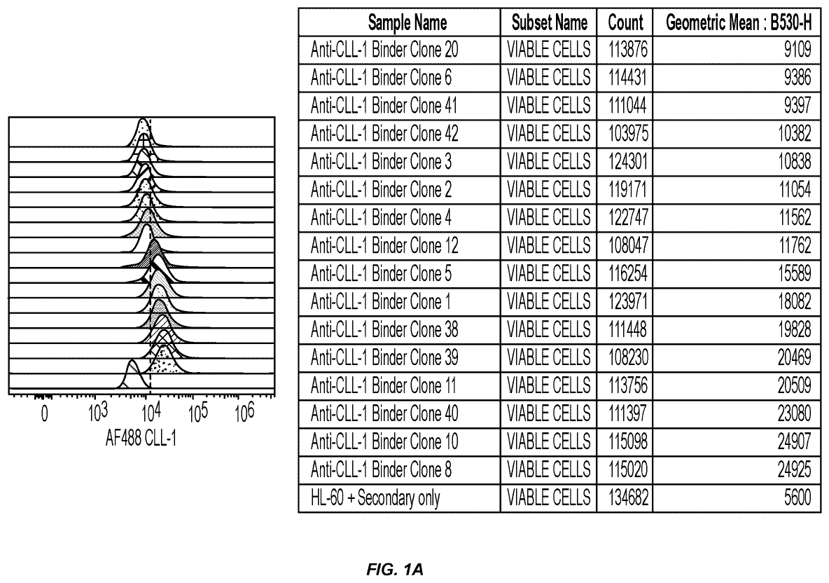

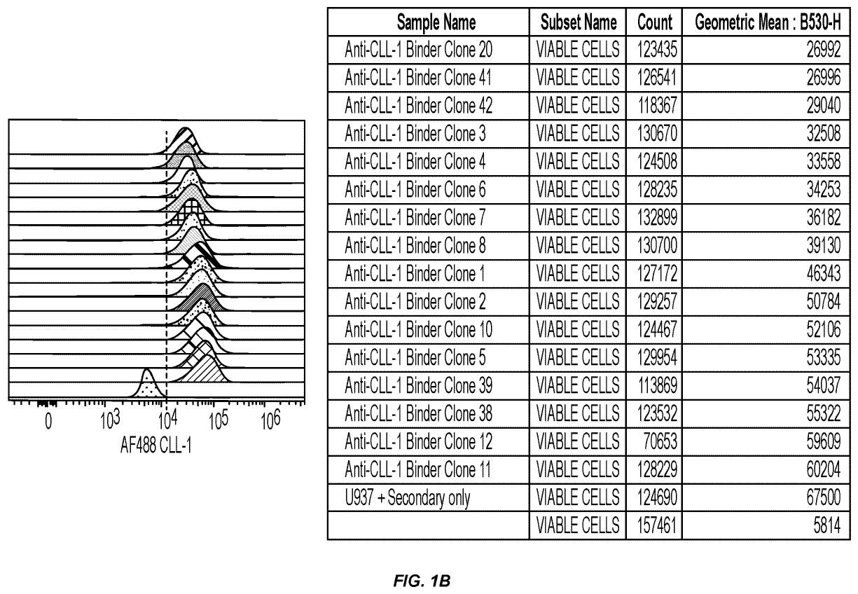

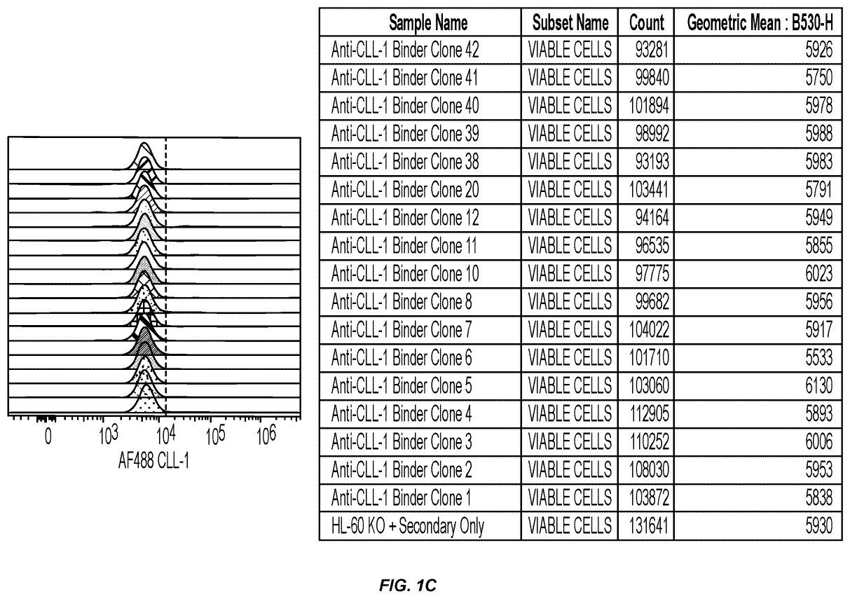

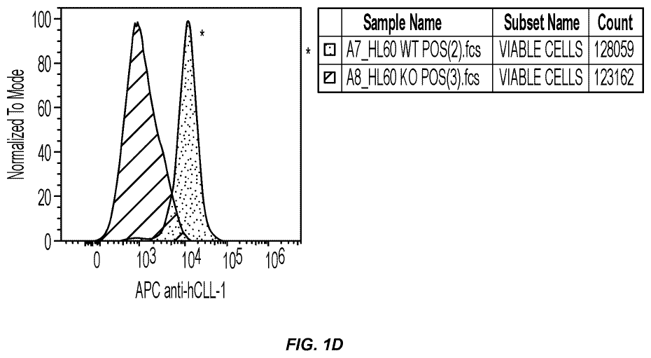

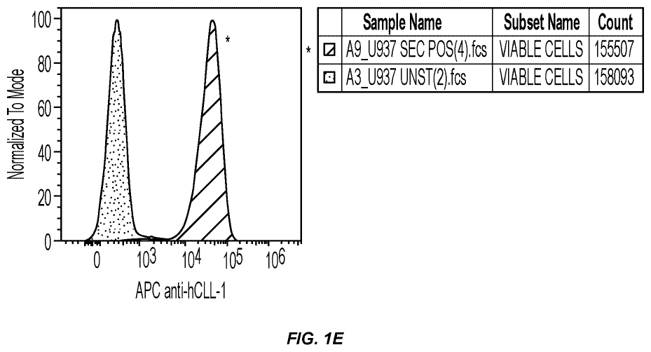

HL60 WT, U937, or HL-60 CLL-1 knockout (KO) control cells were counted, washed in PBS and mixed with 1 ml of Fixable Viability dye eFluor 780 (diluted at 1:1000 in PBS) then incubated at room temperature for 10 minutes to distinguish Live/Dead cells. Subsequently the cells were washed once with FACS Buffer (PBS with 2% FBS) and incubated with human TruStain FcX™ (Fc block diluted at 1:10 in FACS buffer) at room temperature for 5 minutes. Fc blocked cells were stained with 50 μL of the primary CLL-1 antibody (at 200 nM concentrations) and incubated at 4° C. for 30 minutes in the dark. Cells were subsequently stained with 100 μL of secondary antibody (AF488 Rabbit anti-FLAG-tag diluted at 1:1000) incubated at 4° C. for 30 minutes in the dark. Cells were washed and flow cytometric analysis was performed using flow cytometry and data analysis was performed using FlowJo. See FIGS. 1A-1E.

As shown in FIGS. 1A and 1B, CLL-1 antibody binders displayed significantly higher levels of binding to target cells expressing CLL-1, as compared to a secondary antibody only control for both HL60 WT (FIG. 1A) cells and U937 cells (FIG. 1B). FIG. 1C shows CLL-1 antibody binders and secondary antibody only control binding to HL-60 knockout (KO) negative control cells. These data demonstrate specific binding of the panel of CLL-1 binders to AML cell lines.

### Example 2. Generation and Evaluation of CAR Constructs

### CAR Constructs

CAR constructs are developed with CLL-1-specific antibody fragments described herein. Exemplary CAR constructs comprises a single chain antibody or single domain antibody linked with either a CD8α or CD28 transmembrane domain, paired with either a 4-1BB or CD28 co-stimulatory domain, and a CD3ζ (zeta) signaling domain. The CAR sequences are cloned in a third-generation lentiviral plasmid. All restriction enzymes are purchased from New England Biolabs (Ipswich, MA, USA). The sequences of all CAR constructs are confirmed by sequencing. Exemplary CAR construct sequences are provided by SEQ ID NOs: 376-417 and 922-928.

### Cell Lines

The GFP and luciferase expressing AML cells lines MV411, THP1, and MOLM-13, HL-60 and U937 containing varying levels of CLL-1 expression are used to test the efficacy of the CAR constructs described above. MV411 is an acute monocytic leukemia line established from a 10-year-old boy with acute monocytic leukemia (AML FAB M5). THP-1 is a human monocytic cell line derived from an acute monocytic leukemia patient. MOLM-13 is an AML cell line established from the peripheral blood of a 20-year-old man with acute myeloid leukemia (AML FAB M5a) at relapse in 1995 after initial myelodysplastic syndrome (MDS, refractory anemia with excess of blasts, RAEB). Through DNA isolation, it was found that MOLM134 has a CC genotype and does not contain the SNP, while THE1P1 and MV411 are both heterozygous for the SNP with the CT genotype (Lamba et al., J. Clin. Oncol., 35: 2674-82 (2017)). HL-60 is an AML cell line (promyeoloblasts) established from the peripheral blood by leukopheresis of a 36-year-old, Caucasian female with acute promyelocytic leukemia. U937 is an AML cell line derived by Sundstrom and Nilsson in 1974 from malignant cells obtained from the pleural effusion of a patient with histiocytic lymphoma.

### CAR T-Cell Generation

CLL-1 CAR-encoding lentiviral vectors are produced by transient transfection of the Lenti-X 293T lenti packaging cell line Lenti-X 293T cells and plated into poly-D lysine coated 15-cm plates (BD Biosciences, San Jose, CA, USA). The following day, Lenti-X 293T cells are transfected using Lipofectamine™ 3000 (Thermo Fisher Scientific, Waltham, MA, USA) with plasmids encoding the CAR along with packaging and envelope vectors (pMDLg/pRRE, pMD-2G, and pRSV-Rev). Lentiviral supernatants are harvested at 24 and 48 hours post-transfection, centrifuged at 3000 RPM for 10 minutes to remove cell debris, and frozen on dry ice and stored at −80° C. Human PBMCs from normal donors are obtained with an NIH-approved protocol and activated with a 1:3 ratio of CD3/CD28 microbeads (Dynabeads Human T-Expander CD3/CD28, Thermo Fisher Scientific, Cat #11141D) in AIM-V media containing 40 IU/mL recombinant IL-2 and 5% FBS for 24 hours. Activated T cells are re-suspended at 2 million cells per 2 mL of lentiviral supernatant plus 1 mL of fresh AIM-V media with 10 mcg/mL protamine sulfate and 100 IU/mL IL-2 in 6-well plates. Plates are centrifuged at 1000×g for 2 hours at 32° C. and incubated overnight at 37° C. A second transduction is performed on the following day by repeating the same transduction procedure described above. The CD3/CD28 beads are removed on the third day following transduction, and the cells are cultured at 300,000 cells/mL in AIM-V containing 100 IU/mL IL2 with fresh IL2-containing media added every 2-3 days until harvest on day 8 or 9.

### Flow Cytometry Analysis of CLL-1 CAR-T

Surface expression of CLL-1 CAR-transduced T cells is determined by flow cytometry, for example using a Biotinylated Human CLL-1 Protein (Aero Acro Biosystems, Newark, DE, USA) followed by incubation with Streptavidin-PE (BioLegend, San Diego, CA, USA).

### In Vivo Analysis

1 million cells of a leukemia cell line expressing CLL-1 are injected into NOD scid gamma (NSG) mice one week ahead of adoptive CAR T cell transfer. The mice are treated with CAR T cells on day 0. Two weeks later, the mice are sacrificed and analysis is performed.

### Cytotoxicity Assay

5×10⁴ of target tumor cells in 100 μl of RPMI media are loaded into a 96-well plate. An equal amount of CAR T cells are added into the designated well on the following day. The initial Incucyte® apoptosis marker (Essen BioScience, Ann Arbor, MI, USA) is diluted in 100 μL PBS and 1 μL of the diluent is added into each well. The plate is scanned for the GFP and or RFP fluorescent expression to monitor the cell apoptosis using an IncuCyte ZOOM® system every 30 minutes in a duration of 40 hours. The percentage of cell killing at each time point is baseline-corrected.

### Analysis of Cytokine Production

Target tumor cells and transduced CAR-positive T cells are washed 3 times with 1×PBS and re-suspended in RPMI at 1×10⁶/mL. 100 μL of tumor cells with 100 μL of CAR positive T cells are loaded into each well of a 96-well plate. T cell only and tumor cell only controls are set up. All tests are performed in duplicate or triplicate. Cells are incubated for 18 hours at 37° C. and 120 μL of the culture supernatant was harvested for detection of cytokine production. Cytokine levels in supernatants are measured using either ELISA kits (R&D Systems, Minneapolis, MN) or a multiplex assay (Meso Scale Discovery, Rockville, MD, EISA).

### In Vivo Experiments

Animal experiments are carried out under protocols approved by an Animal Care and Use Committee. AML cell lines and the xenografted human AML specimens are intravenously (IV) injected into NSG mice. For luciferase-expressing lines, leukemia is detected using the Xenogen IVIS® Lumina (Caliper Life Sciences, Hopkinton, MA, USA). NSG mice are injected intraperitoneally with 3 mg D-luciferin (Caliper Life Sciences) and are imaged 4 minutes later with an exposure time of 1 min for AML cell lines. Living Image Version 4.1 software (Caliper Life Sciences) is used to analyze the total bioluminescent signal flux for each mouse as photons. At time of take down, bone marrow, spleen, and liver of mice are sacrificed, and cells assessed by flow cytometry.

### Statistical Analysis

Statistics analysis is performed using Prism 7.0 software. Plots are presented as mean+/−SD. Statistical significance of all data is calculated using an unpaired student t test. p<0.05 is considered as significant.

### Example 3. Characterization of CLL-1-CAR T Cells Using a T Cell Activation Reporter System

Exemplary nucleic acid constructs were designed to encode a reporter molecule operably linked to a minimal NFAT-responsive promoter and a second reporter molecule operably linked to a constitutive promoter (e.g., EF1a). The minimal NFAT-responsive promoter contained 6 NFAT binding sites upstream of a minimal IL-2 promoter comprising a TATA box and the coding sequence of the reporter molecule. The nucleic acids were produced using conventional methods known in the art.

The first nucleic acid construct (EF1a_mOrange_IL-2_mTurq) contained the mOrange reporter molecule under control of the constitutively active E1Falpha promoter and mTurquoise reporter molecule (mTurq) under control of the minimal NFAT-responsive promoter. The second nucleic acid construct (EF1a_mTurq_IL-2_mOrange) contained the mTurquoise reporter molecule under control of the constitutively active E1Falpha promoter and mOrange reporter molecule under control of the minimal NFAT-responsive promoter.

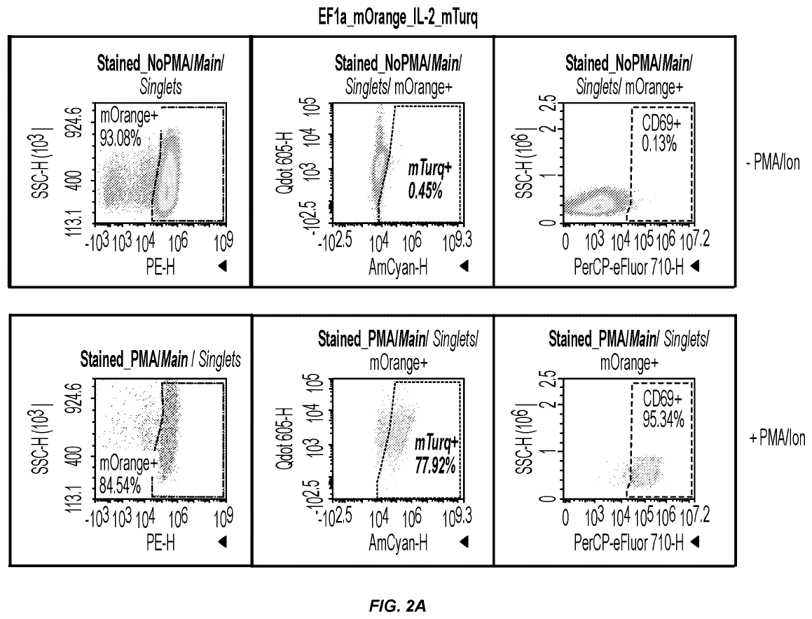

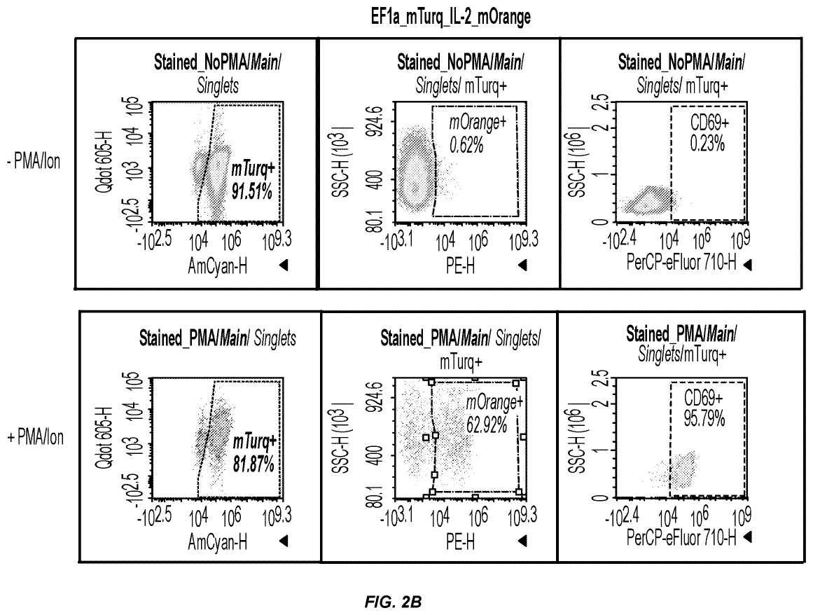

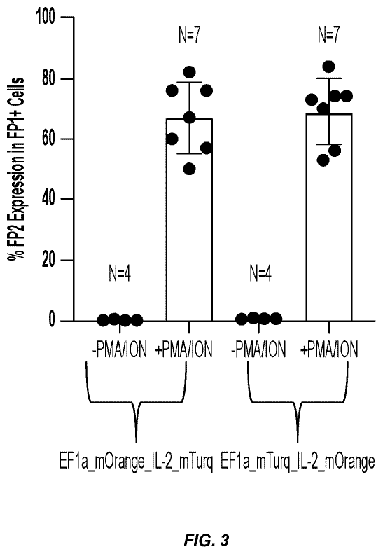

Two IL-2 reporter cell lines were generated by transducing the lentiviral vectors into Jurkat cells. 1×10⁶ cells/mL were activated using 2 μL phorbol myristate acetate (PMA) and ionomycin (a T-cell activation cocktail (see, e.g., BioLegend Activation Cocktail) for 24 hours and assessed for expression of each of the reporter molecules as well as CD69, an indicator of T cell activation, using flow cytometry. As shown in FIGS. 2A and 2B, expression of the reporter molecule under control of the minimal NFAT-responsive promoter was minimally detected when cells are not activated, and significantly increased when cells were activated with PMA/ionomycin. In contrast, expression of the reporter molecule under control of EF1a (the constitutive promoter) was detected in the presence and absence of cell activation. Expression of the reporter molecule under control of the minimal NFAT-responsive promoter was normalized to the expression of the reporter molecule under control of EF1a (the constitutive promoter). See, FIG. 3.

These results indicated that the minimal NFAT-responsive promoter induces expression of the reporter molecule when activated. Expression of the reporter molecule under control of the minimal NFAT-responsive promoter relative to expression of reporter molecule under control of EF1α (the constitutive promoter) provides a means of normalizing expression to account for factors, such as any differing transduction efficiencies between the CLL-1 constructs.

### Example 4. Evaluating CAR Constructs Using a Reporter System

CAR constructs are designed to target CLL-1, as described in Example 2. CLL-1 is a C-type lectin-like receptor that plays a role in regulating granulocyte and monocyte function. CLL-1 is expressed on the surface of the vast majority of AML cells, blast cells, and monocytes, but not on normal hematopoietic stem cells. Currently, treating AML with a therapy that targets CLL-1 can be effective, but the therapy may be limited in utility due to toxicity to normal, healthy cells and tissues. The methods described herein allow for comparison of CAR constructs, such as the activity and function of the CAR constructs, as well as high-throughput screening methods for identifying CAR constructs having desired properties (e.g., level of activation of T cells). Example CAR constructs are known in the art. See, for example, International Publication No. WO 2016/120218 A1, as well as Laborada et al. Int. J. Mol. Sci. (2017) 18(11): 2259.

Reporter cells containing the exemplary nucleic acid construct EF1a_mOrange_IL-2_mTurq or EF1a_mTurq_IL-2_mOrange are generated as described in Example 3. The cells are transduced with the different CLL-1 CARs constructs and are co-cultured for 24 hours with either wild-type MOLM-13 cells (CLL-1+) or MOLM-13 cells that are deficient for CLL-1 (MOLM-13 CLL-1KO).

Following co-culture, expression of the reporter molecules are assessed by flow cytometry. Cells are pre-gated on Jurkat cells displaying the fluorescent marker linked to the EF1α promoter, which indicates cells that are transduced with the nucleic acid construct and are able to express the construct. Next, expression of the IL2 linked fluorescent reporter is determined in each co-culture for each of the CLL-1 CAR constructs as a percentage of constitutive-fluorescence-positive cells (e.g., in cells transduced with EF1a_mOrange_IL-2_mTurq, the expression of mTurq as a percentage of mOrange-positive cells). A ratio is determined for expression of the NFAT-inducible reporter when co-cultured in the presence of wild-type MOLM-13 cells relative to expression of the NFAT-inducible reporter when co-cultured in the presence to MOLM-13 CLL-1KO cells to determine activity of the CLL-1 CAR (CLL-1-specific activation).

Results indicate that the IL-2 reporter system cells can be used as an objective and reliable reporter system for comparing activity of CAR constructs. Assessing expression of a reporter molecule that is constitutively expressed eliminates false outcomes, potentially due to altered transduction efficiencies, and verifies successful transduction of the reporter construct. Expression of the reporter molecule, driven only in activated cells, represents antigen recognition by and activity of the CAR construct.

The extent to which the CLL-1 CARs activate T cells is further evaluated by examining the fold increase in NFAT-inducible fluorescence, the absolute change in NFAT-inducible fluorescence (AFP2), and the degree to which the transduced Jurkat cells express the CLL-1 CAR. As controls, lentiviral vectors encoding known costimulatory or co-inhibitory agents (OX40, ICOS, TIM3, or a VH/VL against CD28) may be transduced into Jurkat cells previously transduced with the EF1a_mOrange_IL-2_mTurq or EF1a_mTurq_IL-2_mOrange construct.

### Example 5. Treatment of Hematologic Disease

An exemplary treatment regimen using the methods, cells, and agents (e.g., a CAR or immune cells expressing a CAR) described herein for AML or MDS is provided. Briefly, a subject having AML or MDS that is a candidate for receiving a hematopoietic stem cell transplant (HSCT) is identified. A suitable HSC donor, e.g., an HLA-matched donor, is identified and HSCs are obtained from the donor, or, if suitable, autologous HSCs from the subject are obtained.

The HSCs so obtained are edited according to the protocols and using the strategies and compositions as described in International Publication Nos. WO/2022/047168 and WO/2022/047165 (both incorporated by reference in their entirety), e.g., a suitable guide RNA targeting a CLL-1 target domain. Briefly, a targeted modification (deletion, truncation, substitution) of CLL-1 is introduced via CRISPR gene editing using a suitable guide RNA and a suitable RNA-guided nuclease, e.g., a Cas9 nuclease, resulting in a loss of CLL-1 expression in at least 80% of the edited HSC population.

The subject having AML or MDS may be preconditioned according to a clinical standard of care, which may include, for example, infusion of chemotherapy agents e.g., etoposide, cyclophosphamide) and/or irradiation. Depending on the health status of the subject and the status of disease progression in the subject, such pre-conditioning may be omitted, however.

T cells expressing a CAR targeting CLL-1 (i.e., CLL-1 CAR-T cells) as described herein is administered to the subject. The edited HSCs from the donor or the edited HSCs from the subject are administered to the subject, and engraftment, survival, and/or differentiation of the HSCs into mature cells of the hematopoietic lineages in the subject are monitored. The CLL-1 CAR-T cells selectively target and kill CLL-1 expressing malignant or pre-malignant cells and may also target some healthy cells expressing CLL-1 in the subject but does not target the edited HSCs or their progeny in the subject, as these cells are resistant to targeting and killing by a CLL-1 CAR-T cells.

The health status and disease progression in the subject is monitored regularly after administration of the CLL-1 CAR-T cells and edited HSCs to confirm a reduction in the burden of CLL-1-expressing malignant or pre-malignant cells, and to confirm successful engraftment of the edited HSCs and their progeny.

### Example 6: Identification and Characterization of CLL-1 Binders

This example describes characterization of binding activities of anti-CLL-1 binders.

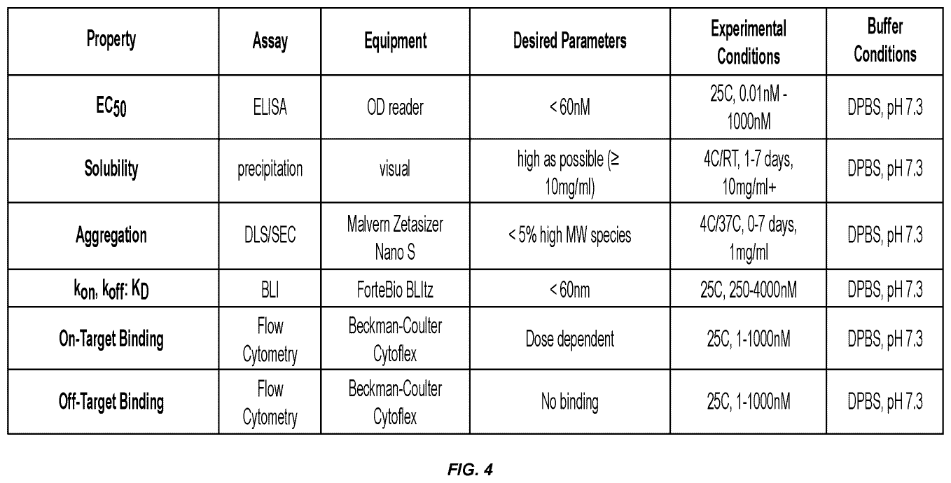

All candidate CLL-1 antibody clones were initially screened using both ELISA and via flow cytometry analyses. The candidates that showed binding activity were then assayed to quantify the binding affinity, for example using biolayer interferometry (BLItz). Properties including half-maximal effective concentration (EC50), solubility, aggregation, binding kinetics, on-targeting binding, and off-target binding were analyzed using the methods summarized in FIG. 4.

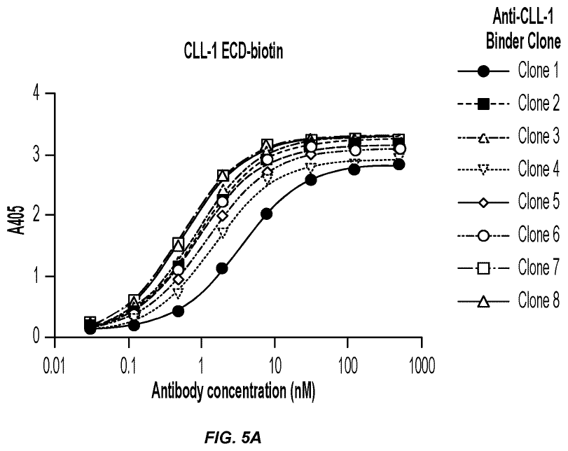

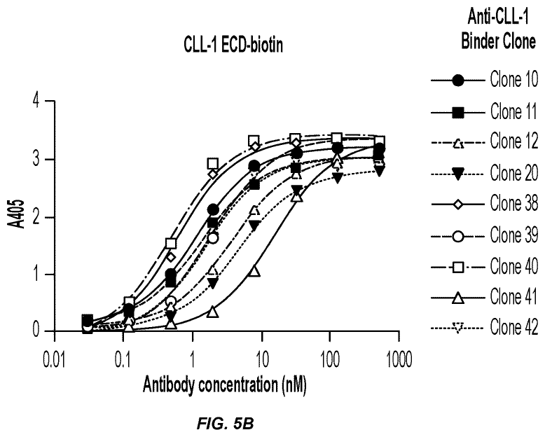

Dose-depending binding activities were analyzed by ELISA. COSTAR® microtiter plates were coated with 1 μg/mL neutravidin protein. Following washing, neutravidin-coated wells were seeded with 100 nM of biotinylated recombinant CLL-1 protein. Following washing, purified FLAG-tagged CLL-1 antibody binders were added to the wells and subsequently detected by mouse anti-FLAG tag HRP conjugate (Sigma). EC50 values were calculated based on measured HRP signal (see, FIGS. 5A-5B and Table 115).

TABLE 115
Half-maximal effective concentration (EC50, nM)
values calculated from ELISA analysis of anti-
CLL-1 antibody binding to recombinant CLL-1.

| Anti-CLL-1 | EC50 |
|---|---|
| Antibody Clone | (nm) |
| 1 | 3.4 |
| 2 | 0.9 |
| 3 | 0.6 |
| 4 | 1.4 |
| 5 | 1.2 |
| 6 | 0.9 |
| 7 | 0.5 |
| 8 | 0.8 |
| 10 | 1.1 |
| 11 | 1.4 |
| 12 | 3.7 |
| 20 VH | 4.7 |
| 38 | 0.6 |
| 39 | 1.9 |
| 40 | 0.5 |
| 41 | 15.0 |
| 42 | 1.5 |

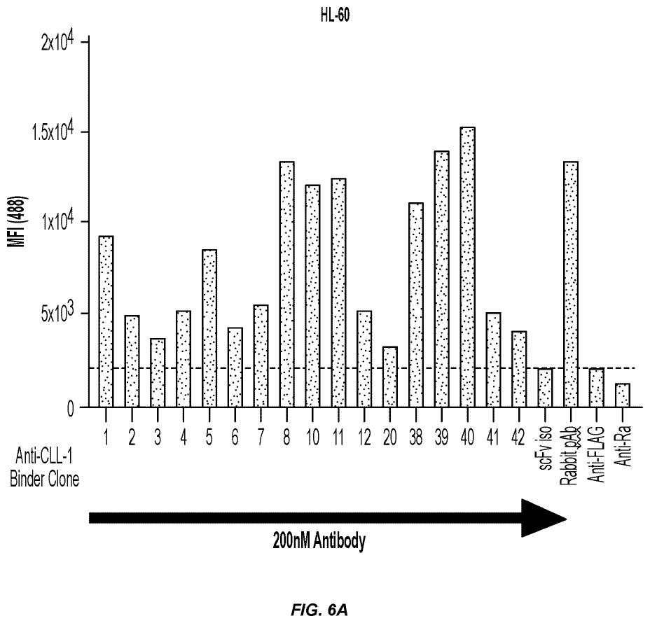

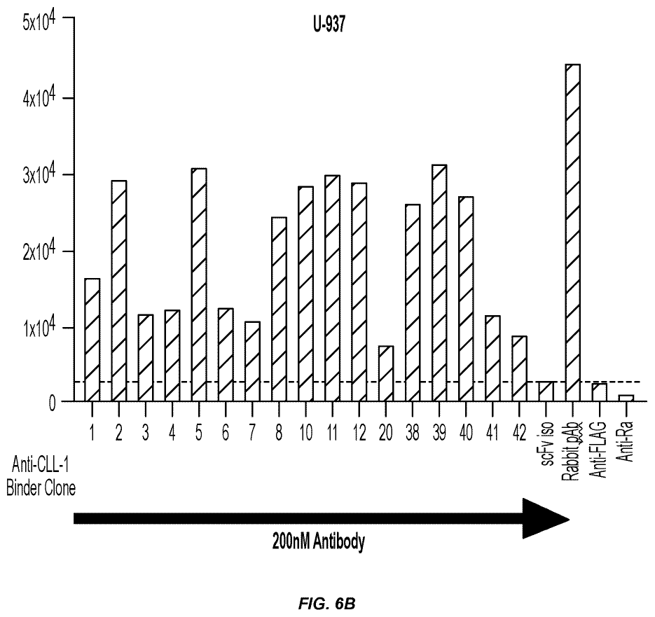

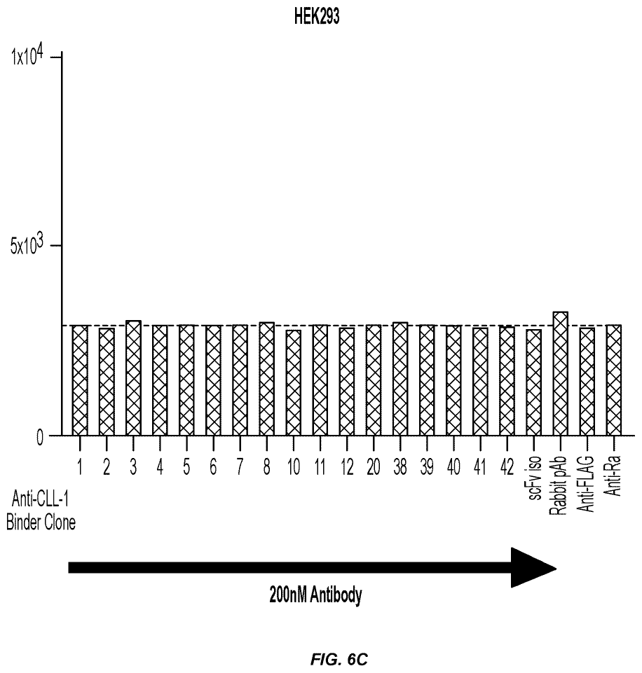

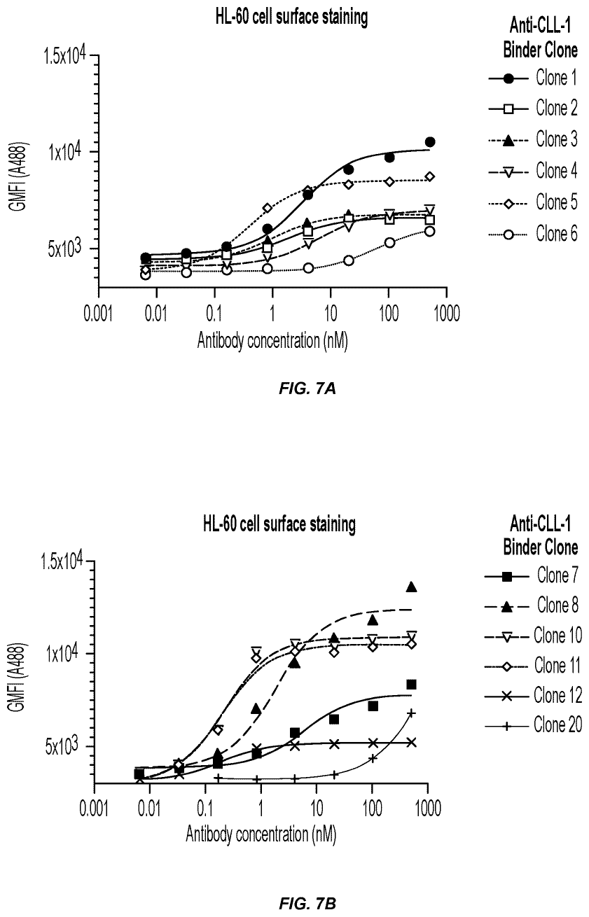

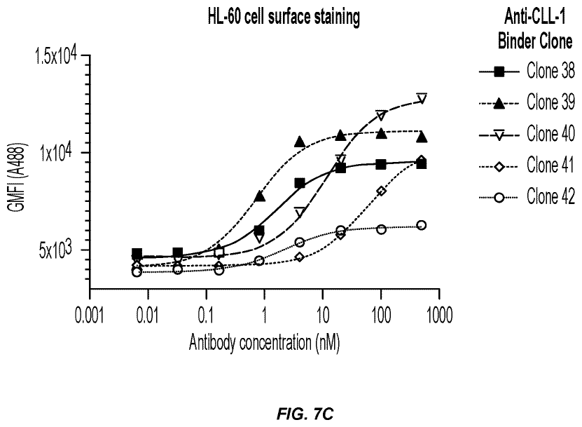

Target cell staining was assessed relative to control samples comprising cells stained with secondary antibodies only (see FIG. 6). Multi-point FACS via flow cytometry was performed to assess dose-depending binding of CLL-1 antibody binders to the cell surface of HL-60 WT cells. Un-fixed, un-permeabilized cells were incubated with various concentrations of purified FLAG-tagged anti-CLL-1 antibody clones and cell-surface binding was detected by subsequent incubation in the presence of rabbit anti-FLAG tag antibody AlexaFluor 488-conjugate antibody (R&D Systems). Fluorescence intensity was calculated as geometric mean for each sample (see, FIGS. 7A-7C) and were used to calculate the EC50 of each CLL-1 antibody binder (see, Table 116).

TABLE 116
Half-maximal effective concentration (EC50, nM) values
calculated from flow cytometry analysis of anti-
CLL-1 antibody binding to recombinant CLL-1.

| Anti-CLL-1 | EC50 |
|---|---|
| Antibody Clone | (nm) |
| 1 | 3.2 |
| 2 | 1.8 |
| 3 | 0.9 |
| 4 | 6.4 |
| 5 | 0.4 |
| 6 | 59 |
| 7 | 6 |
| 8 | 1.8 |
| 10 | 0.2 |
| 11 | 0.2 |
| 12 | 0.2 |
| 20 VH | NA |
| 38 | 1.6 |
| 39 | 0.7 |
| 40 | 11.4 |
| 41 | 57.4 |
| 42 | 2.2 |

ELISAs were performed to characterize the binding of CLL-1 antibody binders to CLL-1 comprising a lysine at amino acid position 244 (CLL-1 K244) and CLL-1 comprising a glutamine at amino acid position 244 (CLL-1 Q244) protein isoforms. For the ELISAs, Nunc™ microtiter plates were coated with 2 μg/mL NeutrAvidin protein. Then, 50 nM of biotinylated recombinant CLL-1 extracellular domain (ECD) protein was bound in 2% BSA blocking buffer. Purified FLAG-tagged CLL-1 antibody binders were added to the wells and detected using mouse anti-FLAG HRP-conjugated secondary antibody (Sigma #A8592). The EC50 of binding of the CLL-1 antibody binders to each antigen (K244, Q244) was calculated, and the reciprocal value was plotted to compare relative affinities.

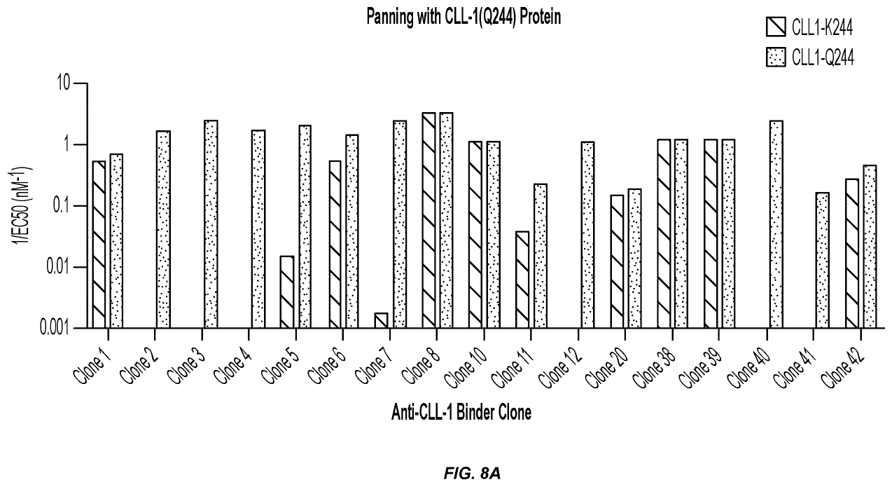

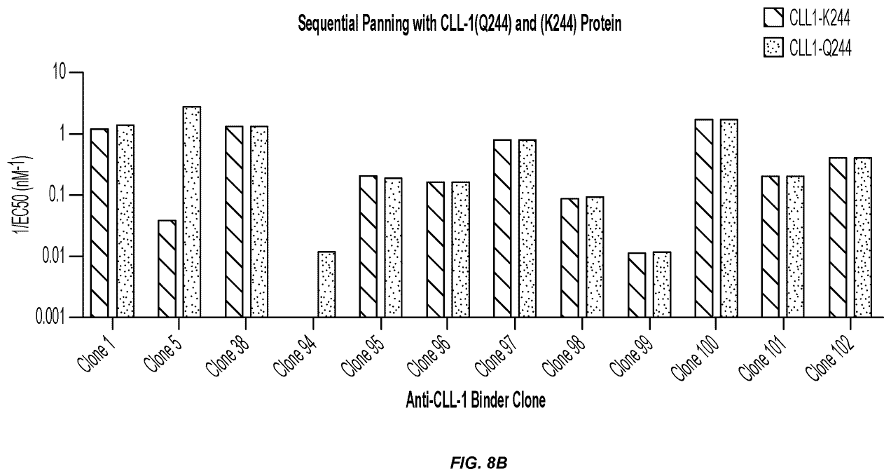

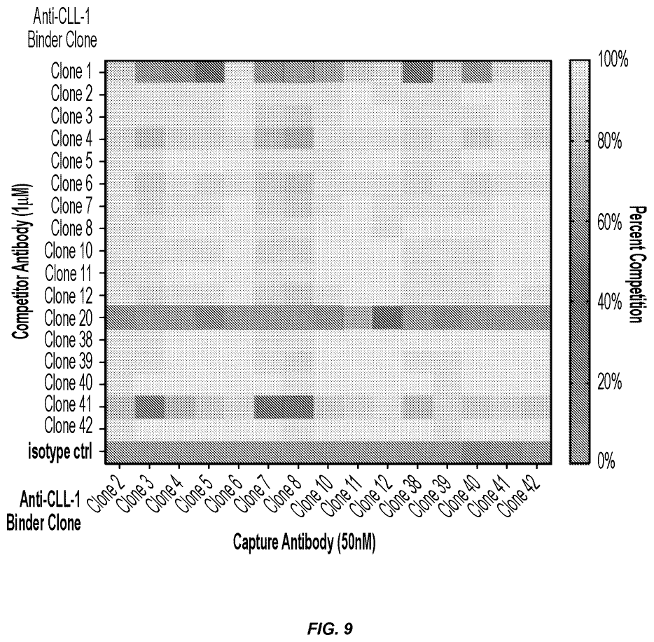

FIG. 8A shows the EC50 of CLL-1 antibody binders that were selected by panning using CLL-1 Q244 isoform. All binders showed reactivity to CLL-1 Q244 (right columns for each indicated binder). Several binders showed binding affinity to CLL-1 K244 (left columns for each indicated binder), indicating the binding for these clones was unaffected by the K/Q variation. Binders that were found to have no significant binding to CLL-1 K244 are dependent on the Q244 isoform of CLL-1.

As shown in FIG. 8B, CLL-1 antibody binders were identified by screening using sequential panning with both CLL-1 K244 and CLL-1 Q244 proteins. This process was used to identify CLL-1 antibody binders that recognize CLL-1 epitopes unaffected by the variation of the amino acid at position 244.

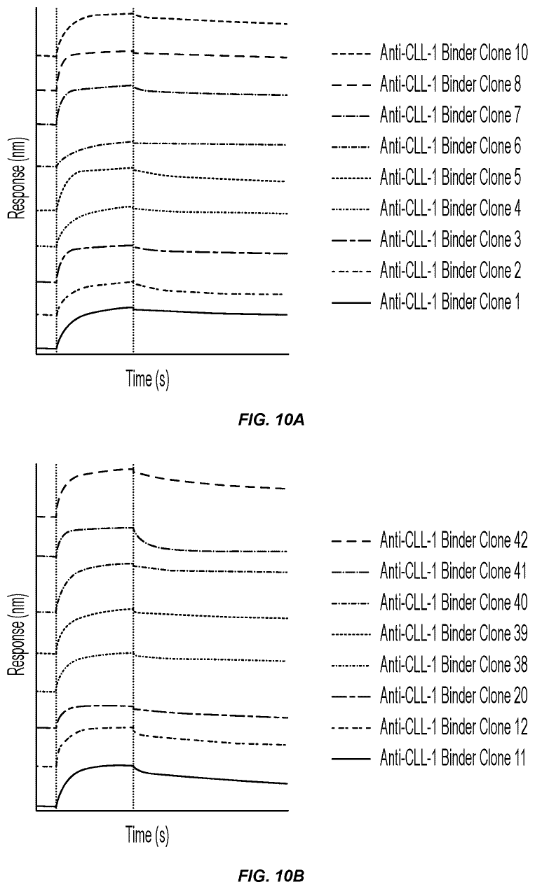

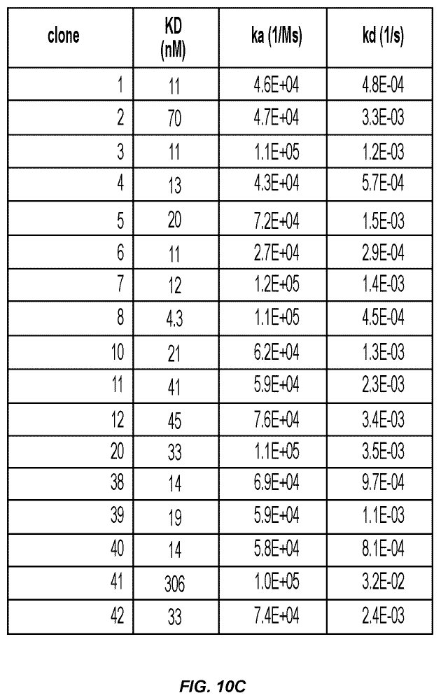

ELISAs were further employed to determine competitive binding activities between CLL-1 antibody binder pairs. Briefly, COSTAR® microtiter plates were coated with 50 nM purified CLL-1 antibody binders. The competitive binding of non-saturating amounts of Fc-tagged CLL-1 ECD protein in the presence of 1 μM competitor antibody was detected by mouse anti-Fc tag HRP conjugate (Sino). Competition was calculated as the percent signal reduction compared to the CLL-1 extracellular domain protein-only (no competitor) control. VH clone 20 showed non-competitive binding with all anti-CLL-1 antibody clones except clone 11. Clones 1 and 41 showed non-competitive binding with select clones (e.g., 8, 39 and 7, 8, respectively) (see, FIGS. 10A and 10B).

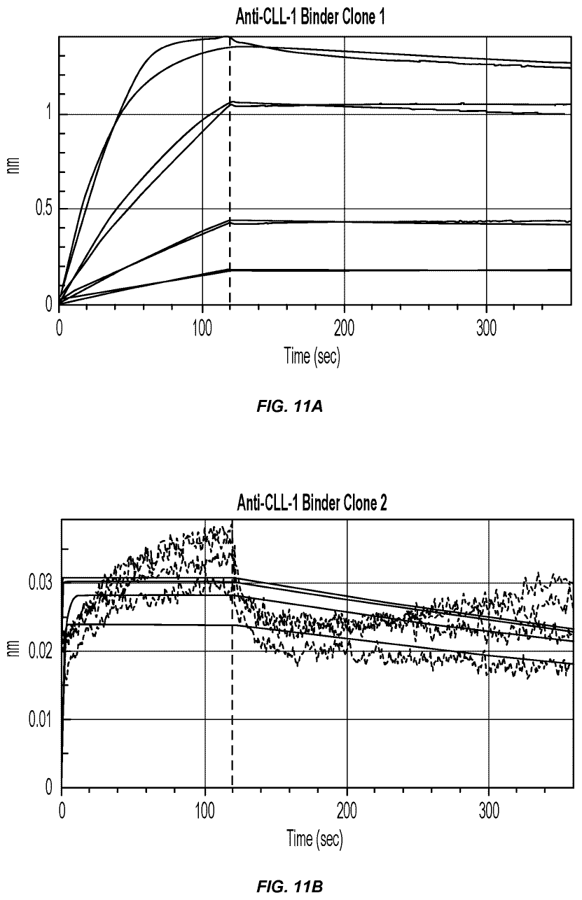

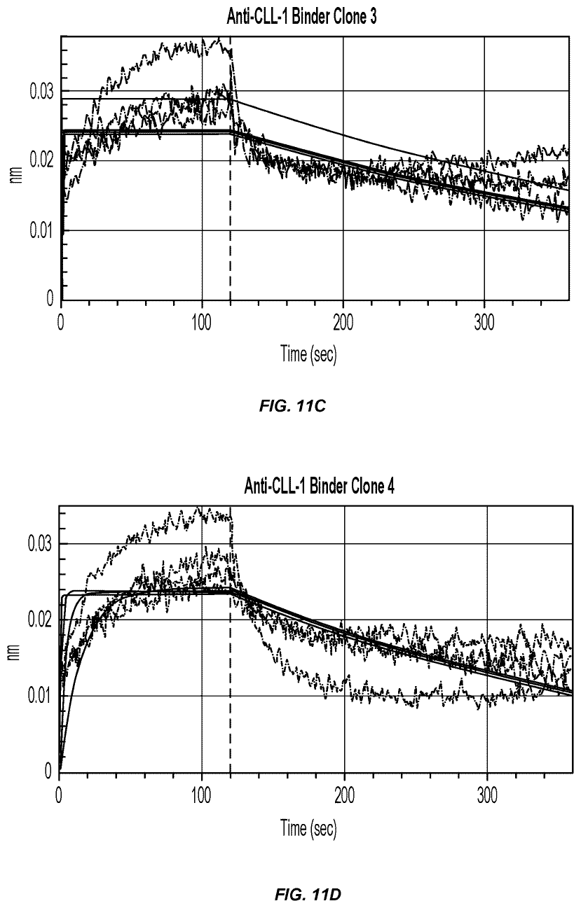

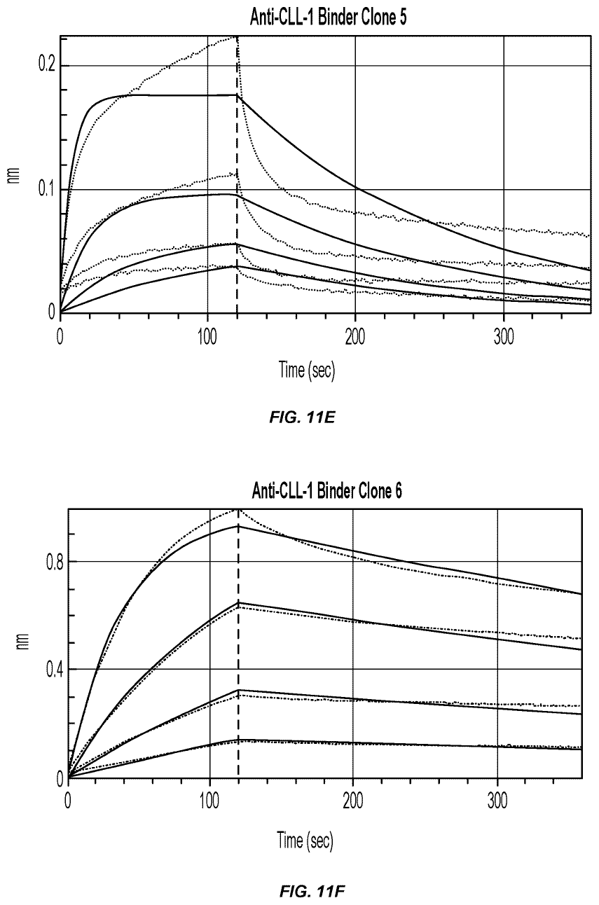

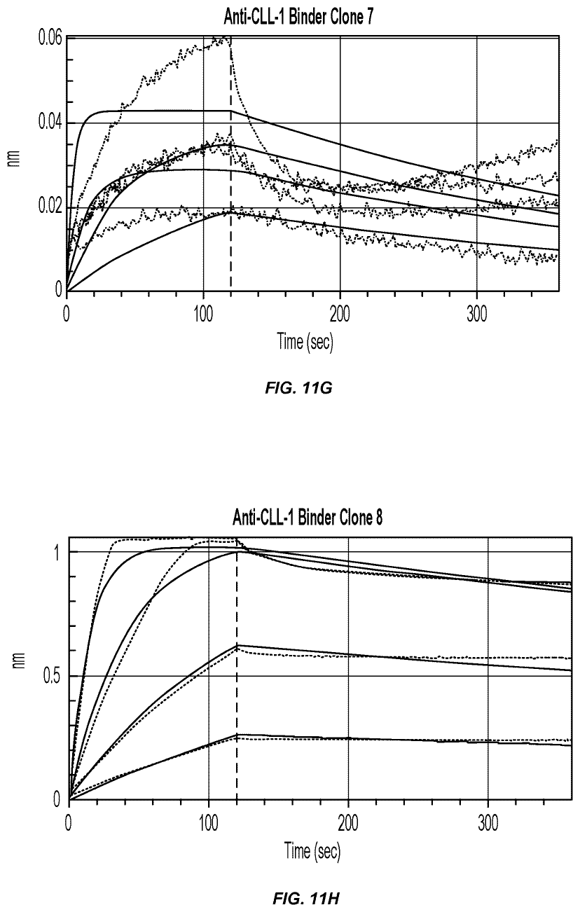

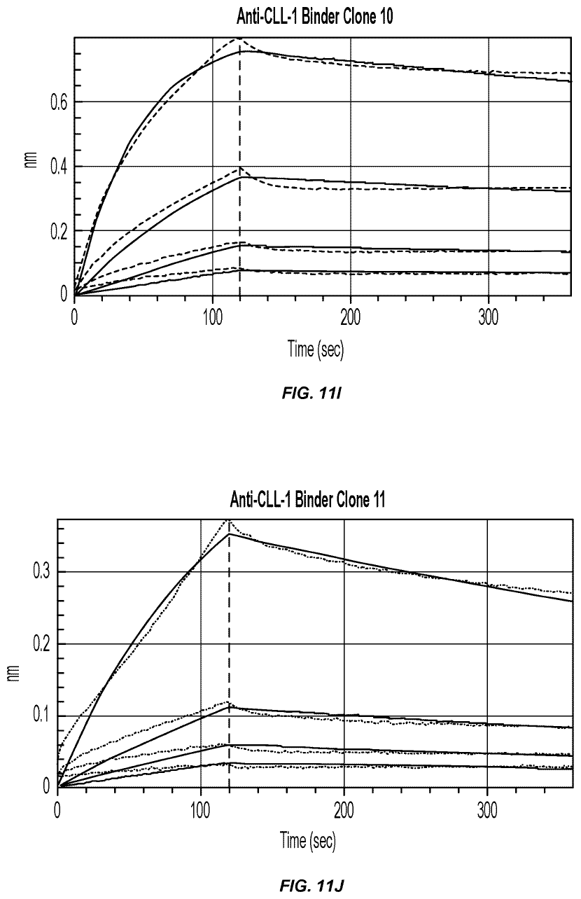

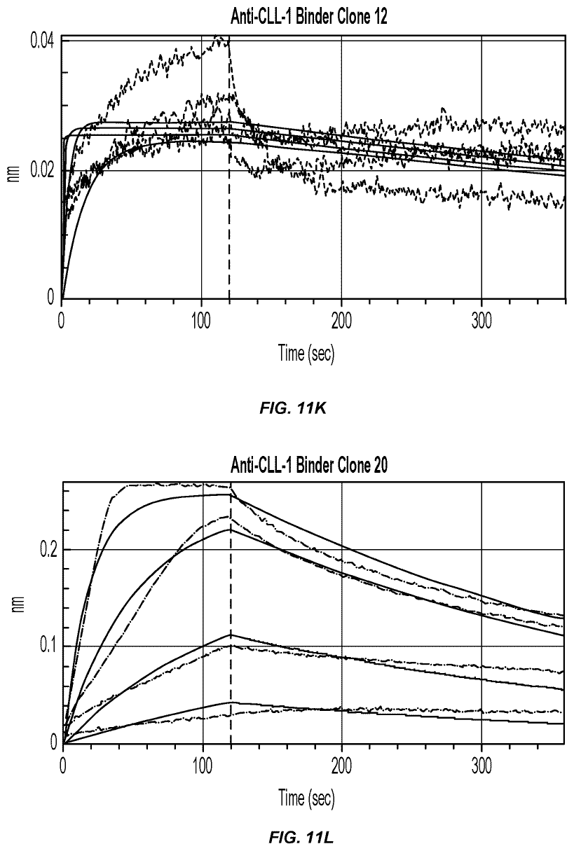

BLItz assays were used to analyze the binding kinetics of CLL-1 antibody binders to recombinant CLL-1 extracellular domain (ECD) protein. Streptavidin-coated biosensors (Sartorius) were loaded with 1 μM biotinylated CLL-1 extracellular domain protein for 4 minutes before being contacted with either 1 μM, 100 nM, or 50 nM of anti-CLL-1 antibody clone for 2 minutes. Dissociation was measured for 4 minutes and kinetic analyses were performed using a 1:1 binding model. Then, binding affinities were calculated (see, FIGS. 10A-10C, and FIG. 11A-11L).

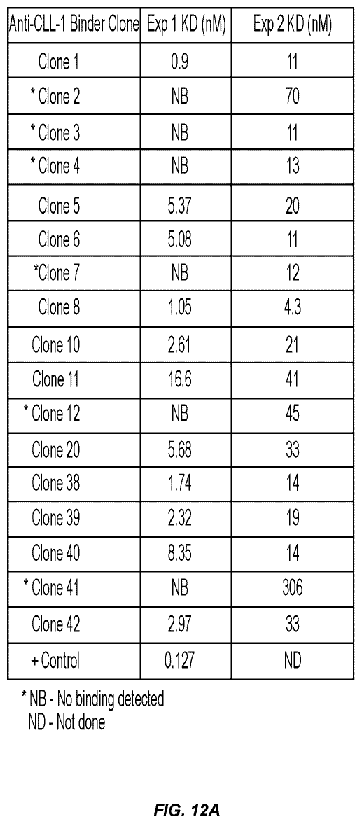

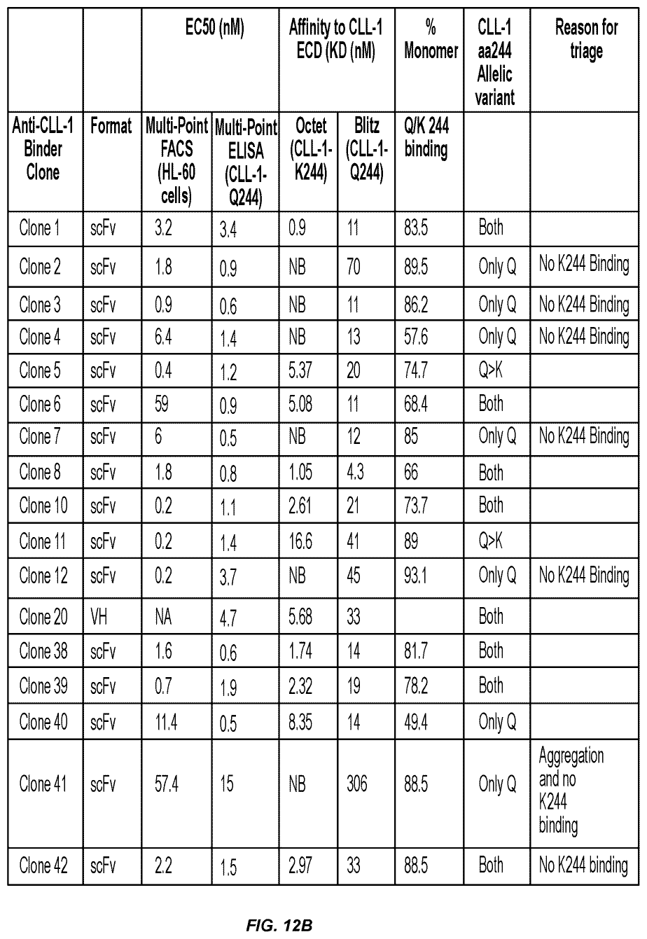

The composite data obtained using clones 1-8, 10-12, and 20 and 38-42 was analyzed. The results indicated clones 2, 3, 4, 7, 12, and 41 showed no binding to recombinant CLL-1 extracellular domain protein. The results also indicated that clones 1, 5, 6, 8, 9, 11, 20, 38, 39, and 42 showed the strongest binding properties. In particular, clone 39 showed the best binding properties to CLL-1 (see, FIGS. 12A and 12B). Exemplary clones, 5, 8, 10, 11, 20, 38, 39, and 42 were used to generate chimeric antigen receptors (CARs).

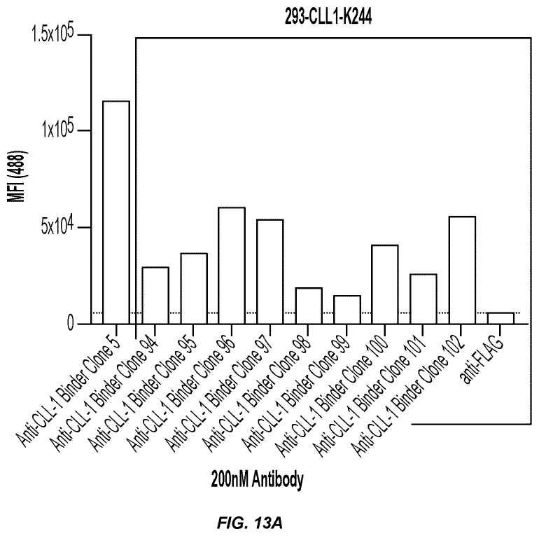

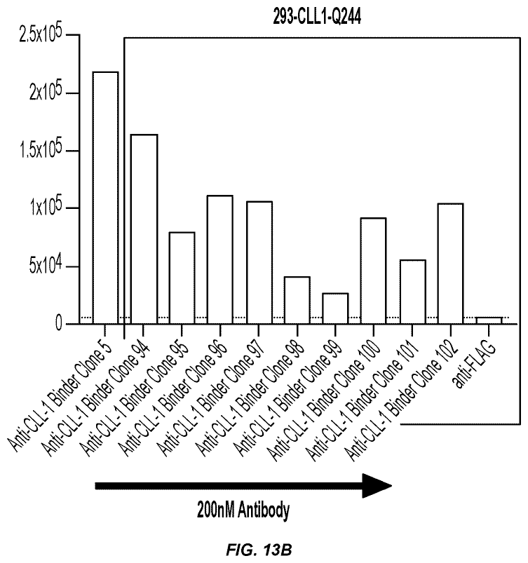

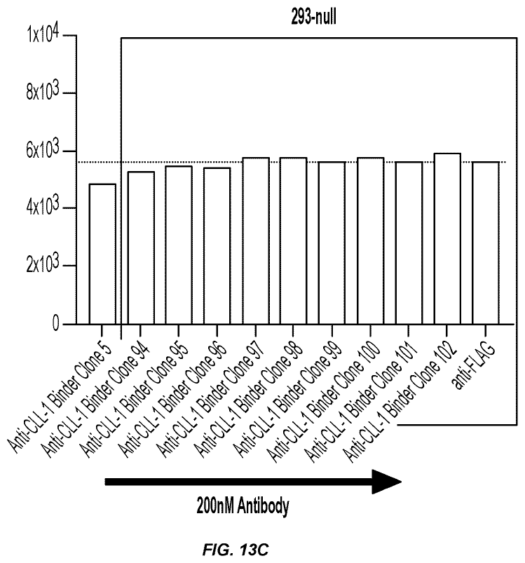

Additional flow cytometry analyses were used to characterize binding of anti-CLL-1 antibody clones. Briefly, HEK293 cells expressing CLL-1-K244, CLL-1-Q244, or no CLL-1 protein were stained with 200 nM of the indicated purified CLL-1 antibody binders. Cell-surface binding activity was detected by rabbit anti-FLAG tag AlexaFluor 488-conjugated antibody (R&D Systems) without fixation or permeabilization. Fluorescence intensity was calculated as the geometric mean for each sample. The anti-CLL-1 antibody clones showed specific binding to the surfaces of cells overexpressing CLL-1 (see, FIGS. 13A-13C).

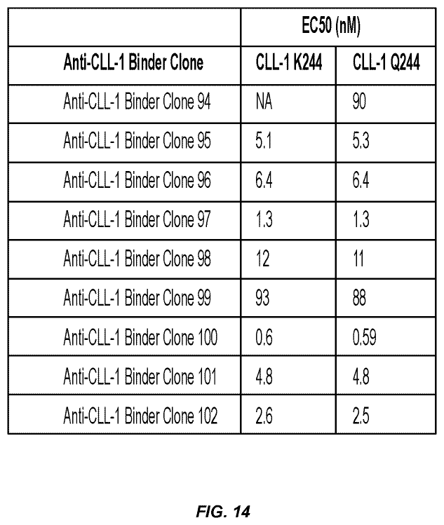

The composite data obtained using exemplary VH-CLL-1 antibody binders was analyzed and select clones were used to generate CAR constructs (see, FIG. 14).

### Example 7: Evaluating CAR-T Cells Comprising CLL-1 Binders

This example describes cell-based assays that were used to characterize activation and cytotoxicity of CLL-1 CAR-T cells.

CLL-1 CAR-T cell activity was evaluated using an IL-2 reporter system (IRS), as described herein. CLL-1 CAR IRS cell lines comprised CD69 and FR2 operably linked to the NFAT-sensitive minimal IL-2 promoter. CLL-1 CAR IRS cells were co-cultured with HL-60 WT cells, HL-60 CLL-1 KO cells, or alone, at a 1:1 ratio for 24 hours. Specific activation of CLL-1 CAR IRS cells was determined by analysis of CD69 and FR2 expression via flow cytometry (see, FIG. 15A). FR2 detection was determined by IncuCyte live cell imaging analysis (see, FIG. 15B). These results demonstrated antigen-specific activation of CLL-1 CAR-T cells as a result of co-culture with HL-60 WT cells.

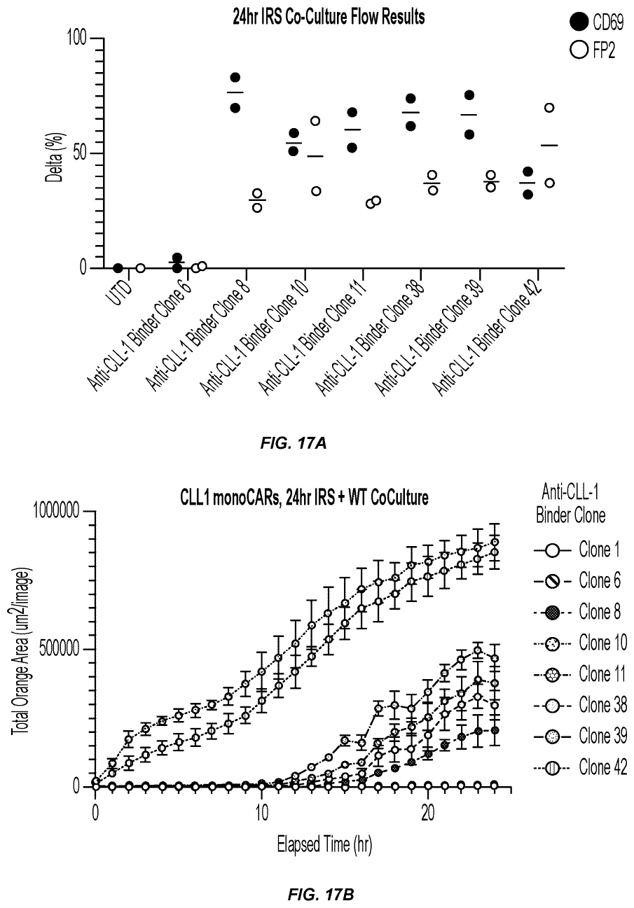

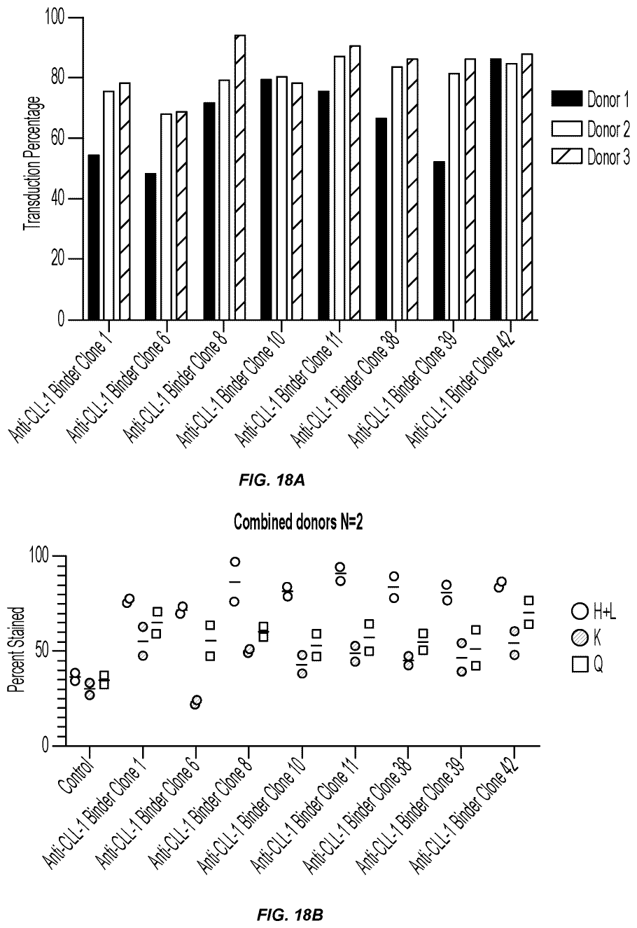

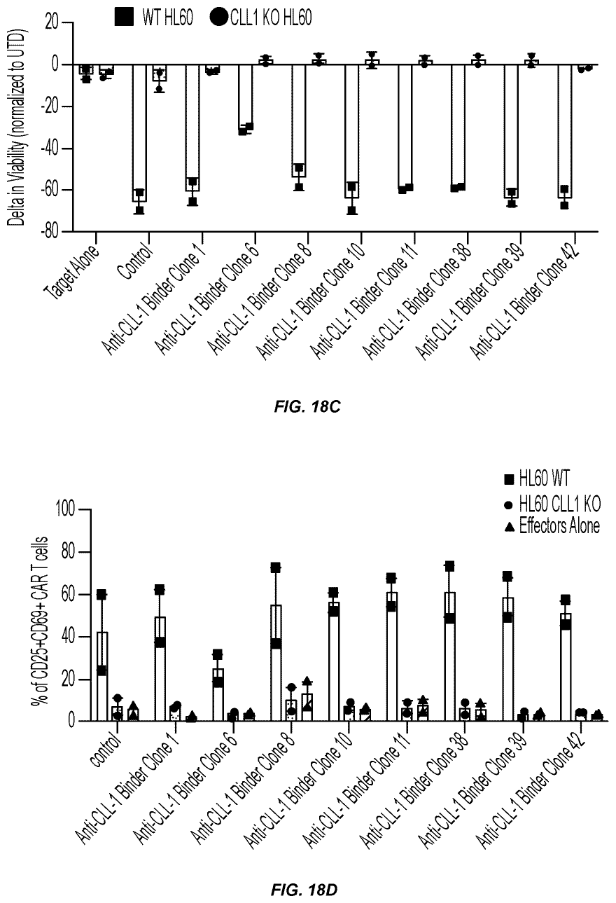

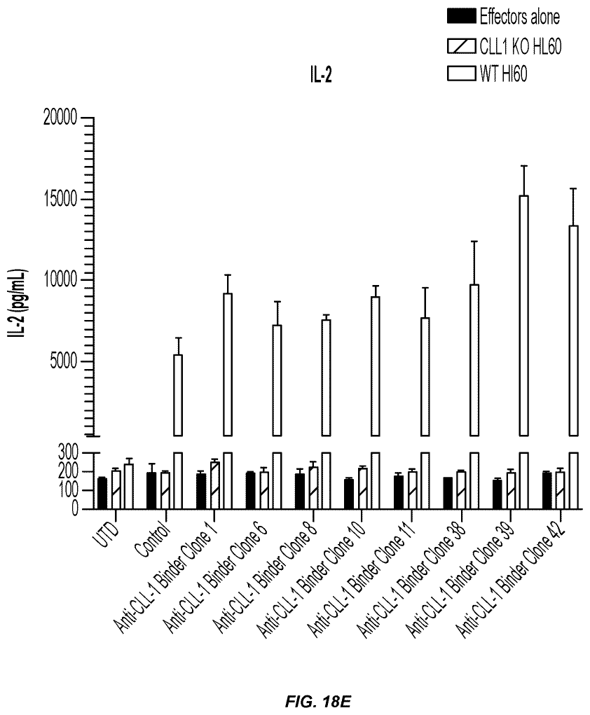

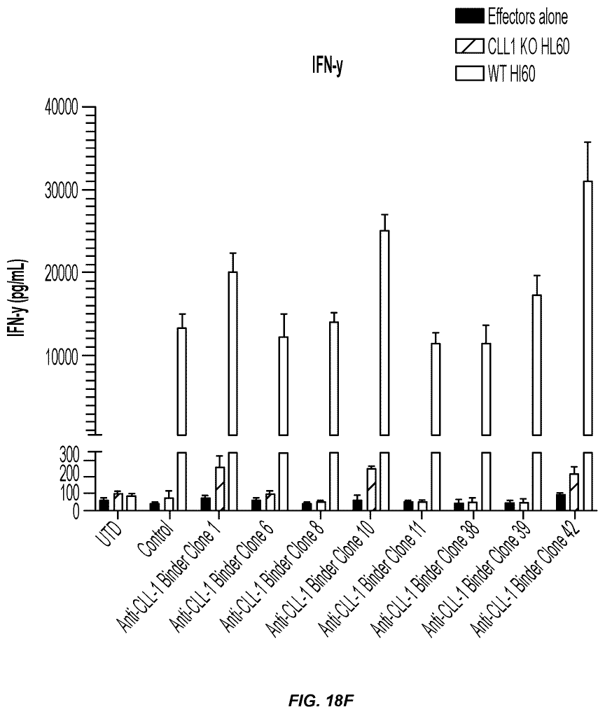

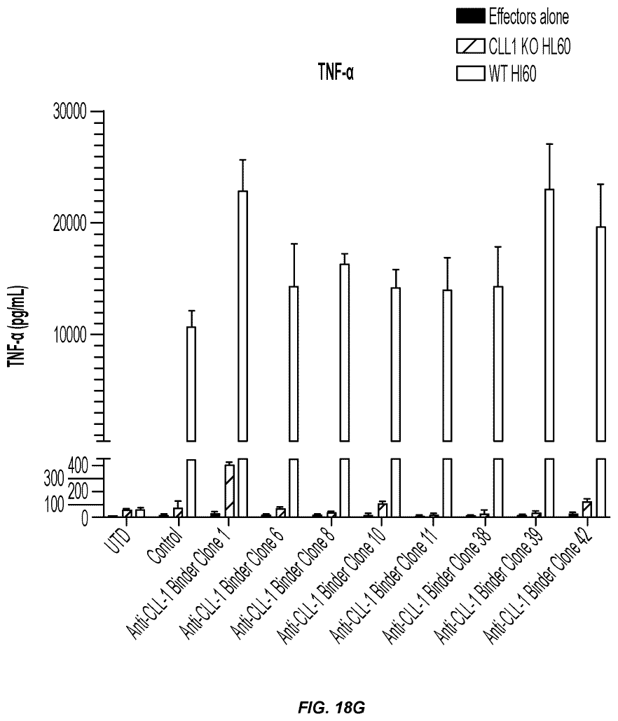

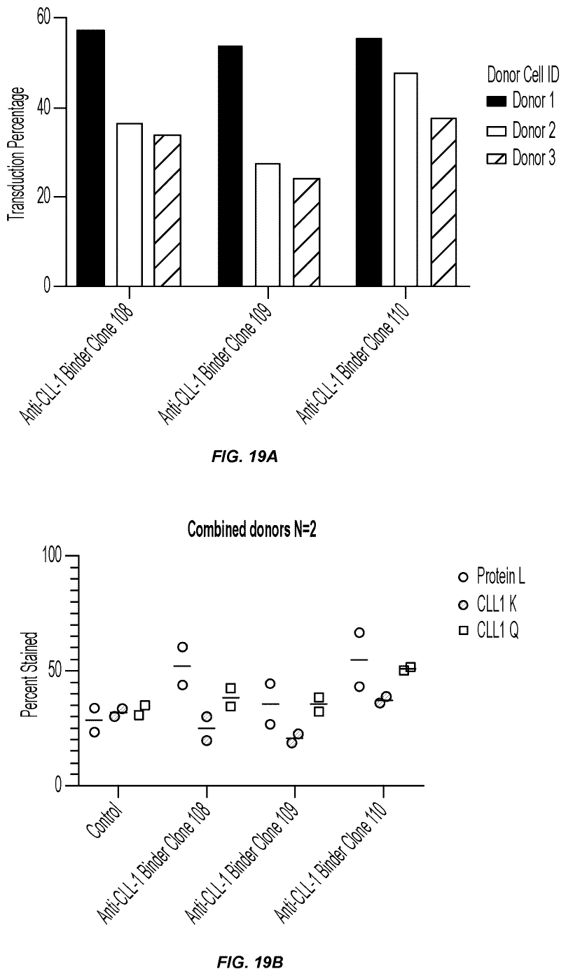

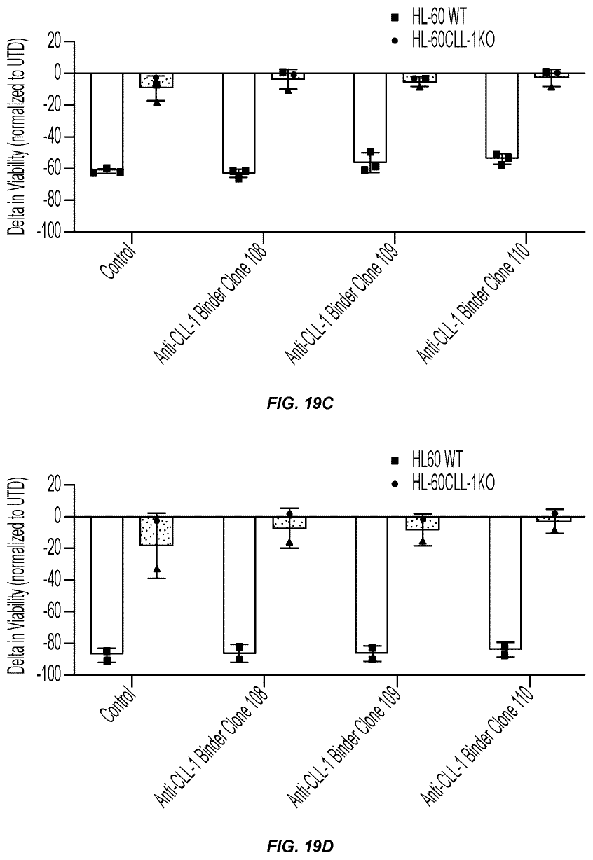

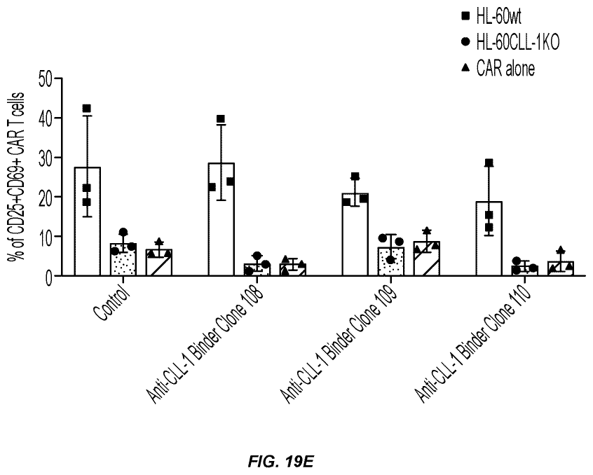

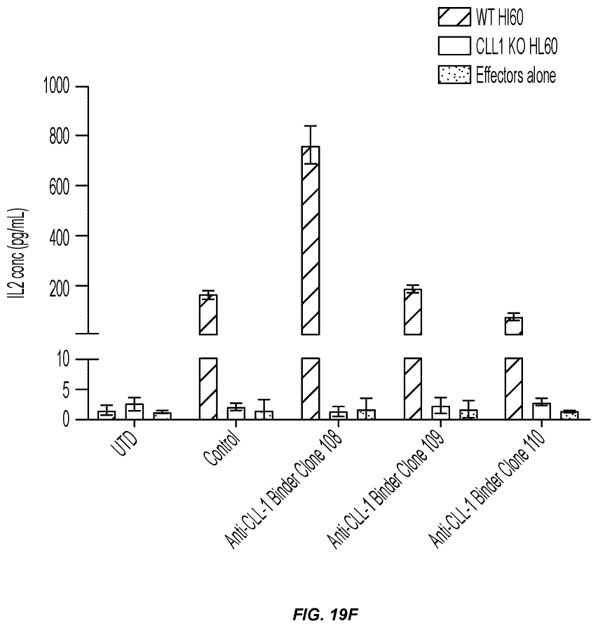

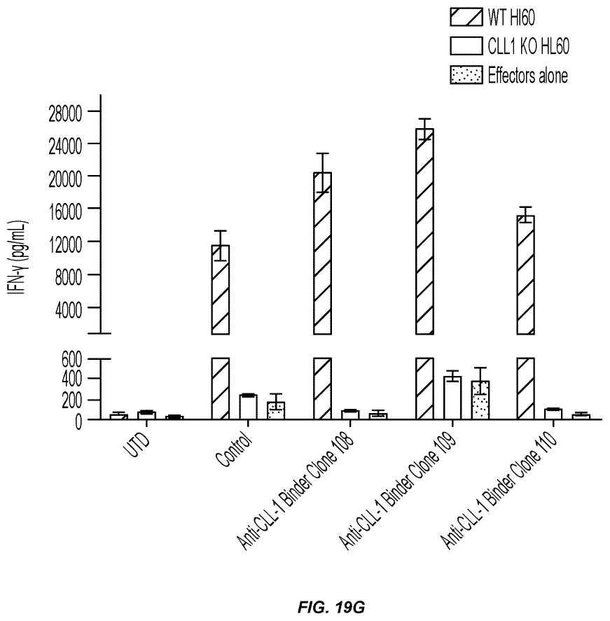

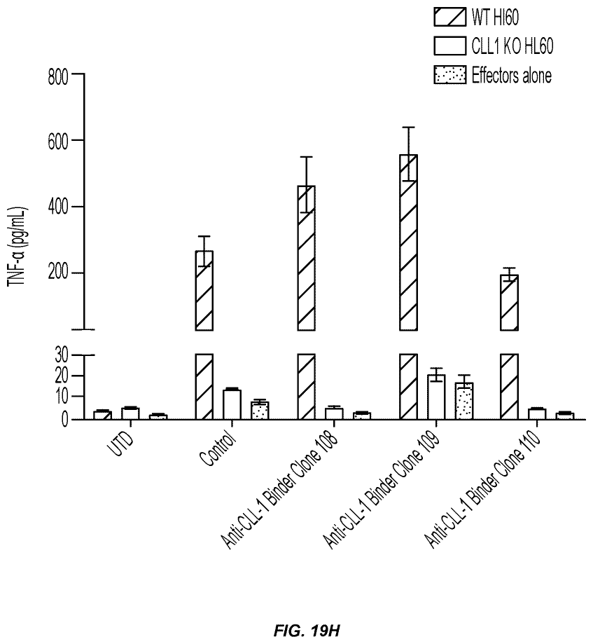

Transduction efficiency of primary T cells with the CLL-1 CAR constructs was determined by anti-human IgG (H+L) reactivity using flow cytometry (see, FIG. 16A) or by Protein L reactivity via flow cytometry (see, FIG. 17A). Cell-surface binding analyses determined via flow cytometry indicated that CLL-1 CAR-T cells bound both K244 and Q244 CLL-1 isoforms on the surface of target cells (see, FIGS. 16B and 16B). First, the cytotoxicity of the CLL-1 CAR T cells was assessed. Briefly, carboxyfluorescein succinimidyl ester (CFSE)-labeled target cells—HL-60 WT cells or HL-60 CLL-1 KO cells—were co-cultured with the CLL-1 CAR T cells at a ratio of 1:1. The percentage of viable target cells was determined by the absence of Annexin V and Fixable Viability dye reactivity as measured by flow cytometry and normalized to donor-matched untransduced cells (UTD). The results indicated a high amount of CLL-1 antigen-specific target cell killing (see FIGS. 16C, 17C, and 17D). In addition, CD25 and CD69 expression were analyzed via flow cytometry. The results indicated rapid CLL-1 CAR-T cell activation in nearly all populations co-cultured with CFSE-labeled HL-60 WT cells (see, Table 116 and FIG. 17E). Additionally, cytokine analysis was performed following co-culture. Levels of each of IL-2, IFN-γ, and TNF-α in the supernatant of co-cultures was determined by Luminex® Assay and indicated increased cytokine levels from CLL-1 CAR-T cells co-cultured with HL-60 WT cells (see, FIGS. 16E-16G and 17F-17G).

All references, including publications, patent applications, and patents, cited herein are hereby incorporated by reference to the same extent as if each reference were individually and specifically indicated to be incorporated by reference and were set forth in its entirety herein.

### EQUIVALENTS AND SCOPE

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which two or more members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods described herein, can be excluded from any one or more claims. For purposes of brevity, all of the embodiments in which one or more elements, features, purposes, or aspects is excluded are not set forth.

1. An anti-CLL-1 antibody, or antigen-binding fragment thereof, comprising an amino acid sequence of SEQ ID NOs: 15, 29, 43, 57, 71, 85, 99, 113, 127, 141, 155, 169, 174, 179, 184, 189, 194, 199, 204, 209, 214, 219, 224, 229, 234, 239, 244, 249, 254, 259, 264, 269, 275, 280, 285, 290, 295, 309, 323, 337, 351, 365, 418, 422, 426, 430, 434, 438, 442, 446, 450, 454, 458, 462, 466, 470, 474, 478, 482, 486, 490, 494, 498, 502, 506, 510, 514, 518, 522, 526, 530, 534, 538, 542, 546, 550, 554, 558, 562, 566, 570, 574, 578, 582, 586, 590, 594, 598, 602, 606, 610, 614, 618, 622, 626, 630, 634, 638, 642, 646, 650, 654, 658, 662, 666, 670, 674, 678, 682, 686, 690, 694, 698, 702, 706, 710, 714, 718, 722, 726, 730, 734, 738, 742, 746, 750, 754, 758, 762, 766, 774, 779, 784, 788, 793, 798, 801, 804, 808, 819, 831, 837, 849, 900, 905, 910, 918 or 920.

2. An anti-CLL-1 antibody, or antigen-binding fragment thereof, comprising at least one complementarity determining region (CDR) sequence of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 820-825, 838-843, 850-854, 861-866, 896-898, 901-903, 906-908, or 911-916.

3. The anti-CLL-1 antibody, or antigen-binding fragment thereof, of claim 2, comprising three CDR sequences of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 820-825, 838-843, 850-854, 861-866, 896-898, 901-903, 906-908, or 911-916.

4. The anti-CLL-1 antibody, or antigen-binding fragment thereof, of claim 2, comprising six CDR sequences of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 820-825, 838-843, 850-854, 861-866, 896-898, 901-903, 906-908, or 911-916.

5. The anti-CLL-1 antibody, or antigen-binding fragment thereof, of claim 2, comprising three heavy chain CDR sequences of SEQ ID NOs: 5-7, 19-21, 33-35, 47-49, 61-63, 75-77, 89-91, 103-105, 117-119, 131-133, 145-147, 159-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 299-301, 313-315, 327-329, 341-343, 355-357, 423-425, 431-433, 439-441, 447-449, 455-457, 463-465, 471-473, 479-481, 487-489, 495-497, 503-505, 511-513, 519-521, 527-529, 535-537, 543-545, 551-553, 559-561, 567-569, 575-577, 583-585, 591-593, 599-601, 607-609, 615-617, 623-625, 631-633, 639-641, 647-649, 655-657, 663-665, 671-673, 679-681, 687-689, 695-697, 703-705, 711-713, 715-746, 748-808, 816-817, 823-825, 828-829, 834-835, 841-843, 846-847, 852-854, 857-858, 869-870, 880-881, 892-893, 896-910, 914-916, or 919-920.

6. An anti-CLL-1 antibody, or antigen-binding fragment thereof, comprising a CDR1, CDR2, and CDR3 of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 820-825, 838-843, 850-854, 896-898, 901-903, 906-908, or 911-916.

7. An anti-CLL-1 antibody, or antigen-binding fragment thereof, comprising at least one CDR that is at least 85%, 90%, 91%, 92%, 93%, 94%, 95%, 96%, 97%, 98%, 99%, or 100% identical to a CDR (e.g., CDR1, CDR2, and/or CDR3) of SEQ ID NOs: 2-7, 16-21, 30-35, 44-49, 58-63, 72-77, 86-91, 100-105, 114-119, 128-133, 142-147, 156-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 296-301, 310-315, 324-329, 338-343, 352-357, 419-421, 423-425, 427-429, 431-433, 435-437, 439-441, 443-445, 447-449, 451-453, 455-457, 459-461, 463-465, 467-469, 471-473, 475-477, 479-481, 483-485, 487-489, 491-493, 495-497, 499-501, 503-505, 507-509, 511-513, 515-517, 519-521, 523-525, 527-529, 531-533, 535-537, 539-541, 543-545, 547-549, 551-553, 555-557, 559-561, 563-565, 567-569, 571-573, 575-577, 579-581, 583-585, 587-589, 591-593, 595-597, 599-601, 603-605, 607-609, 611-613, 615-617, 619-621, 623-625, 627-629, 631-633, 635-637, 639-641, 643-645, 647-649, 651-653, 655-657, 659-661, 663-665, 667-669, 671-673, 675-677, 679-681, 683-685, 687-689, 691-693, 695-697, 699-701, 703-705, 707-709, 711-713, 715-717, 719-721, 723-725, 727-729, 731-733, 735-737, 739-741, 743-745, 747-749, 751-753, 755-757, 759-761, 763-765, 767-769, 770-772, 775-777, 780-782, 785-786, 789-791, 794-796, 799, 802, 805-806, 820-825, 838-843, 850-854, 896-898, 901-903, 906-908, or 911-916.

8. An anti-CLL-1 antibody, or antigen-binding fragment thereof, comprising
a heavy chain variable region comprising a CDR1 provided by SEQ ID NO: 5, 19, 33, 47, 61, 75, 89, 103, 117, 131, 145, 159, 170, 175, 180, 185, 190, 195, 200, 205, 210, 215, 220, 225, 230, 235, 240, 245, 250, 255, 260, 265, 271, 276, 281, 286, 291, 299, 313, 327, 341, 355, 423, 431, 439, 447, 455, 463, 471, 479, 487, 495, 503, 511, 519, 527, 535, 543, 551, 559, 567, 575, 583, 591, 599, 607, 615, 623, 631, 639, 647, 655, 663, 671, 679, 687, 695, 703, 711, 715, 719, 723, 727, 731, 735, 739, 743, 747, 751, 755, 759, 763, 767, 770, 775, 780, 785, 789, 794, 823, 841, 852, 896, 901, 906, or 914;
a CDR2 provided by SEQ ID NO: 6, 20, 34, 48, 62, 76, 90, 104, 118, 132, 146, 160, 171, 176, 181, 186, 191, 196, 201, 206, 211, 216, 221, 226, 231, 236, 241, 246, 251, 256, 261, 266, 272, 277, 282, 287, 292, 300, 314, 328, 342 356, 424, 432, 440, 448, 456, 464, 472, 480, 488, 496, 504, 512, 520, 528, 536, 544, 552, 560, 568, 576, 584, 592, 600, 608, 616, 624, 632, 640, 648, 656, 664, 672, 680, 688, 696, 704, 712, 716, 720, 724, 728, 732, 736, 740, 744, 748, 752, 756, 760, 764, 768, 771, 776, 781, 786, 790, 795, 799, 802, 805, 824, 842, 853, 897, 902, 907, or 915; and
a CDR3 provided by SEQ ID NO: 7, 21, 35, 49, 63, 77, 91, 105, 119, 133, 147, 161, 172, 177, 182, 187, 192, 197, 202, 207, 212, 217, 222, 227, 232, 237, 242, 247, 252, 257, 262, 267, 273, 278, 283, 288, 293, 301, 315, 329, 343, 357, 425, 433, 441, 449, 457, 465, 473, 481, 489, 497, 505, 513, 521, 529, 537, 545, 553, 561, 569, 577, 585, 593, 601, 609, 617, 625, 633, 641, 649, 657, 665, 673, 681, 689, 697, 705, 713, 717, 721, 725, 729, 733, 737, 741, 745, 749, 753, 757, 761, 765, 769, 772, 777, 782, 791, 796, 806, 825, 843, 854, 898, 903, 908, or 916; and
a light chain variable region comprising a CDR1 provided by SEQ ID NO: 2, 16, 30, 44, 58, 72, 86, 100, 114, 128, 142, 156, 296, 310, 324, 338, 352, 419, 427, 435, 443, 451, 459, 467, 475, 483, 491, 499, 507, 515, 523, 531, 539, 547, 555, 563, 571, 579, 587, 595, 603, 611, 619, 627, 635, 643, 651, 659, 667, 675, 683, 691, 699, 707, 820, 838, 850, or 911,
a CDR2 provided by SEQ ID NO: 3, 17, 31, 45, 59, 73, 87, 101, 115, 129, 143, 157, 297, 311, 325, 339, 353, 420, 428, 436, 444, 452, 460, 468, 476, 484, 492, 500, 508, 516, 524, 532, 540, 548, 556, 564, 572, 580, 588, 596, 604, 612, 620, 628, 636, 644, 652, 660, 668, 676, 684, 692, 700, 708, 821, 839, or 912, and
a CDR3 provided by SEQ ID NO: 4, 18, 32, 46, 60, 74, 88, 102, 116, 130, 144, 158, 298, 312, 326, 340, 354, 421, 429, 437, 445, 453, 461, 469, 477, 485, 493, 501, 509, 517, 525, 533, 541, 549, 557, 565, 573, 581, 589, 597, 605, 613, 621, 629, 637, 645, 653, 661, 669, 677, 685, 693, 701, 709, 822, 840, 851, or 913.

9. The anti-CLL-1 antibody, or antigen-binding fragment thereof, of any one of claims 2-8, comprising:
a heavy chain variable region comprising a CDR1 provided by SEQ ID NO: 5, a CDR2 provided by SEQ ID NO: 6, and a CDR3 provided by SEQ ID NO: 7; and
a light chain variable region comprising a CDR1 provided by SEQ ID NO: 2, a CDR2 provided by SEQ ID NO: 3, and a CDR3 provided by SEQ ID NO: 4.

10. The anti-CLL-1 antibody, or antigen-binding fragment thereof, of any one of claims 2-8, comprising:
a heavy chain variable region comprising a CDR1 provided by SEQ ID NO: 19, a CDR2 provided by SEQ ID NO: 20, and a CDR3 provided by SEQ ID NO: 21; and
a light chain variable region comprising a CDR1 provided by SEQ ID NO: 16, a CDR2 provided by SEQ ID NO: 17, and a CDR3 provided by SEQ ID NO: 18.

11. The anti-CLL-1 antibody, or antigen-binding fragment thereof, of any one of claims 2-8, comprising:
a heavy chain variable region comprising a CDR1 provided by SEQ ID NO: 33, a CDR2 provided by SEQ ID NO: 34, and a CDR3 provided by SEQ ID NO: 35; and
a light chain variable region comprising a CDR1 provided by SEQ ID NO: 30, a CDR2 provided by SEQ ID NO: 31, and a CDR3 provided by SEQ ID NO: 32.

12. The anti-CLL-1 antibody, or antigen-binding fragment thereof, of any one of claims 2-8, comprising:
a heavy chain variable region comprising a CDR1 provided by SEQ ID NO: 47, a CDR2 provided by SEQ ID NO: 48, and a CDR3 provided by SEQ ID NO: 49; and
a light chain variable region comprising a CDR1 provided by SEQ ID NO: 44, a CDR2 provided by SEQ ID NO: 45, and a CDR3 provided by SEQ ID NO: 46.

13. The anti-CLL-1 antibody, or antigen-binding fragment thereof, of any one of claims 2-8, comprising:
a heavy chain variable region comprising a CDR1 provided by SEQ ID NO: 61, a CDR2 provided by SEQ ID NO: 62, and a CDR3 provided by SEQ ID NO: 63; and
a light chain variable region comprising a CDR1 provided by SEQ ID NO: 58, a CDR2 provided by SEQ ID NO: 59, and a CDR3 provided by SEQ ID NO: 60.

14. The anti-CLL-1 antibody, or antigen-binding fragment thereof, of any one of claims 2-8, comprising:
a heavy chain variable region comprising a CDR1 provided by SEQ ID NO: 75, a CDR2 provided by SEQ ID NO: 76, and a CDR3 provided by SEQ ID NO: 77; and
a light chain variable region comprising a CDR1 provided by SEQ ID NO: 72, a CDR2 provided by SEQ ID NO: 73, and a CDR3 provided by SEQ ID NO: 74.

15. The anti-CLL-1 antibody, or antigen-binding fragment thereof, of any one of claims 2-8, comprising:
a heavy chain variable region comprising a CDR1 provided by SEQ ID NO: 89, a CDR2 provided by SEQ ID NO: 90, and a CDR3 provided by SEQ ID NO: 91; and
a light chain variable region comprising a CDR1 provided by SEQ ID NO: 86, a CDR2 provided by SEQ ID NO: 87, and a CDR3 provided by SEQ ID NO: 88.

16. The anti-CLL-1 antibody, or antigen-binding fragment thereof, of any one of claims 2-8, comprising:
a heavy chain variable region comprising a CDR1 provided by SEQ ID NO: 103, a CDR2 provided by SEQ ID NO: 104, and a CDR3 provided by SEQ ID NO: 105; and
a light chain variable region comprising a CDR1 provided by SEQ ID NO: 100, a CDR2 provided by SEQ ID NO: 101, and a CDR3 provided by SEQ ID NO: 102.

17. The anti-CLL-1 antibody, or antigen-binding fragment thereof, of any one of claims 2-8, comprising:
a heavy chain variable region comprising a CDR1 provided by SEQ ID NO: 117, a CDR2 provided by SEQ ID NO: 118, and a CDR3 provided by SEQ ID NO: 119; and
a light chain variable region comprising a CDR1 provided by SEQ ID NO: 114, a CDR2 provided by SEQ ID NO: 115, and a CDR3 provided by SEQ ID NO: 116.

18. The anti-CLL-1 antibody, or antigen-binding fragment thereof, of any one of claims 2-8, comprising:
a heavy chain variable region comprising a CDR1 provided by SEQ ID NO: 131, a CDR2 provided by SEQ ID NO: 132, and a CDR3 provided by SEQ ID NO: 133; and
a light chain variable region comprising a CDR1 provided by SEQ ID NO: 128, a CDR2 provided by SEQ ID NO: 129, and a CDR3 provided by SEQ ID NO: 130.

19. The anti-CLL-1 antibody, or antigen-binding fragment thereof, of any one of claims 2-8, comprising:
a heavy chain variable region comprising a CDR1 provided by SEQ ID NO: 145, a CDR2 provided by SEQ ID NO: 146, and a CDR3 provided by SEQ ID NO: 147; and
a light chain variable region comprising a CDR1 provided by SEQ ID NO: 142, a CDR2 provided by SEQ ID NO: 143, and a CDR3 provided by SEQ ID NO: 144.

20. The anti-CLL-1 antibody, or antigen-binding fragment thereof, of any one of claims 2-8, comprising:
a heavy chain variable region comprising a CDR1 provided by SEQ ID NO: 159, a CDR2 provided by SEQ ID NO: 160, and a CDR3 provided by SEQ ID NO: 161; and
a light chain variable region comprising a CDR1 provided by SEQ ID NO: 156, a CDR2 provided by SEQ ID NO: 157, and a CDR3 provided by SEQ ID NO: 158.

21. The anti-CLL-1 antibody, or antigen-binding fragment thereof, of any one of claims 2-8, comprising:
a heavy chain variable region comprising a CDR1 provided by SEQ ID NO: 299, a CDR2 provided by SEQ ID NO: 300, and a CDR3 provided by SEQ ID NO: 301; and
a light chain variable region comprising a CDR1 provided by SEQ ID NO: 296, a CDR2 provided by SEQ ID NO: 297, and a CDR3 provided by SEQ ID NO: 298.

22. The anti-CLL-1 antibody, or antigen-binding fragment thereof, of any one of claims 2-8, comprising:
a heavy chain variable region comprising a CDR1 provided by SEQ ID NO: 313, a CDR2 provided by SEQ ID NO: 314, and a CDR3 provided by SEQ ID NO: 315; and
a light chain variable region comprising a CDR1 provided by SEQ ID NO: 310, a CDR2 provided by SEQ ID NO: 311, and a CDR3 provided by SEQ ID NO: 312.

23. The anti-CLL-1 antibody, or antigen-binding fragment thereof, of any one of claims 2-8, comprising:
a heavy chain variable region comprising a CDR1 provided by SEQ ID NO: 327, a CDR2 provided by SEQ ID NO: 328, and a CDR3 provided by SEQ ID NO: 329; and
a light chain variable region comprising a CDR1 provided by SEQ ID NO: 324, a CDR2 provided by SEQ ID NO: 325, and a CDR3 provided by SEQ ID NO: 326.

24. The anti-CLL-1 antibody, or antigen-binding fragment thereof, of any one of claims 2-8, comprising:
a heavy chain variable region comprising a CDR1 provided by SEQ ID NO: 341, a CDR2 provided by SEQ ID NO: 342, and a CDR3 provided by SEQ ID NO: 343; and
a light chain variable region comprising a CDR1 provided by SEQ ID NO: 338, a CDR2 provided by SEQ ID NO: 339, and a CDR3 provided by SEQ ID NO: 340.

25. The anti-CLL-1 antibody, or antigen-binding fragment thereof, of any one of claims 2-8, comprising:
a heavy chain variable region comprising a CDR1 provided by SEQ ID NO: 355, a CDR2 provided by SEQ ID NO: 356, and a CDR3 provided by SEQ ID NO: 357; and
a light chain variable region comprising a CDR1 provided by SEQ ID NO: 352, a CDR2 provided by SEQ ID NO: 353, and a CDR3 provided by SEQ ID NO: 354.

26. The anti-CLL-1 antibody, or antigen-binding fragment thereof, of any one of claims 2-8, comprising:
a heavy chain variable region comprising a CDR1 provided by SEQ ID NO: 423, a CDR2 provided by SEQ ID NO: 424, and a CDR3 provided by SEQ ID NO: 425; and
a light chain variable region comprising a CDR1 provided by SEQ ID NO: 418, a CDR2 provided by SEQ ID NO: 419, and a CDR3 provided by SEQ ID NO: 421.

27. The anti-CLL-1 antibody, or antigen-binding fragment thereof, of any one of claims 2-8, comprising:
a heavy chain variable region comprising a CDR1 provided by SEQ ID NO: 431, a CDR2 provided by SEQ ID NO: 432, and a CDR3 provided by SEQ ID NO: 433; and
a light chain variable region comprising a CDR1 provided by SEQ ID NO: 427, a CDR2 provided by SEQ ID NO: 428, and a CDR3 provided by SEQ ID NO: 429.

28. The anti-CLL-1 antibody, or antigen-binding fragment thereof, of any one of claims 2-8, comprising:
a heavy chain variable region comprising a CDR1 provided by SEQ ID NO: 439, a CDR2 provided by SEQ ID NO: 440, and a CDR3 provided by SEQ ID NO: 441; and
a light chain variable region comprising a CDR1 provided by SEQ ID NO: 435, a CDR2 provided by SEQ ID NO: 436, and a CDR3 provided by SEQ ID NO: 437.

29. The anti-CLL-1 antibody, or antigen-binding fragment thereof, of any one of claims 2-8, comprising:
a heavy chain variable region comprising a CDR1 provided by SEQ ID NO: 447, a CDR2 provided by SEQ ID NO: 448, and a CDR3 provided by SEQ ID NO: 449; and
a light chain variable region comprising a CDR1 provided by SEQ ID NO: 443, a CDR2 provided by SEQ ID NO: 444, and a CDR3 provided by SEQ ID NO: 445.

30. The anti-CLL-1 antibody, or antigen-binding fragment thereof, of any one of claims 2-8, comprising:
a heavy chain variable region comprising a CDR1 provided by SEQ ID NO: 455, a CDR2 provided by SEQ ID NO: 456, and a CDR3 provided by SEQ ID NO: 457; and
a light chain variable region comprising a CDR1 provided by SEQ ID NO: 451, a CDR2 provided by SEQ ID NO: 452, and a CDR3 provided by SEQ ID NO: 453.

31. The anti-CLL-1 antibody, or antigen-binding fragment thereof, of any one of claims 2-8, comprising:
a heavy chain variable region comprising a CDR1 provided by SEQ ID NO: 463, a CDR2 provided by SEQ ID NO: 464, and a CDR3 provided by SEQ ID NO: 465; and
a light chain variable region comprising a CDR1 provided by SEQ ID NO: 459, a CDR2 provided by SEQ ID NO: 460, and a CDR3 provided by SEQ ID NO: 461.

32. The anti-CLL-1 antibody, or antigen-binding fragment thereof, of any one of claims 2-8, comprising:
a heavy chain variable region comprising a CDR1 provided by SEQ ID NO: 471, a CDR2 provided by SEQ ID NO: 472, and a CDR3 provided by SEQ ID NO: 473; and
a light chain variable region comprising a CDR1 provided by SEQ ID NO: 467, a CDR2 provided by SEQ ID NO: 468, and a CDR3 provided by SEQ ID NO: 469.

33. The anti-CLL-1 antibody, or antigen-binding fragment thereof, of any one of claims 2-8, comprising:
a heavy chain variable region comprising a CDR1 provided by SEQ ID NO: 479, a CDR2 provided by SEQ ID NO: 480, and a CDR3 provided by SEQ ID NO: 481; and
a light chain variable region comprising a CDR1 provided by SEQ ID NO: 475, a CDR2 provided by SEQ ID NO: 476, and a CDR3 provided by SEQ ID NO: 477.

34. The anti-CLL-1 antibody, or antigen-binding fragment thereof, of any one of claims 2-8, comprising:
a heavy chain variable region comprising a CDR1 provided by SEQ ID NO: 487, a CDR2 provided by SEQ ID NO: 488, and a CDR3 provided by SEQ ID NO: 489; and
a light chain variable region comprising a CDR1 provided by SEQ ID NO: 483, a CDR2 provided by SEQ ID NO: 484, and a CDR3 provided by SEQ ID NO: 485.

35. The anti-CLL-1 antibody, or antigen-binding fragment thereof, of any one of claims 2-8, comprising:
a heavy chain variable region comprising a CDR1 provided by SEQ ID NO: 495, a CDR2 provided by SEQ ID NO: 496, and a CDR3 provided by SEQ ID NO: 497; and
a light chain variable region comprising a CDR1 provided by SEQ ID NO: 491, a CDR2 provided by SEQ ID NO: 492, and a CDR3 provided by SEQ ID NO: 493.

36. The anti-CLL-1 antibody, or antigen-binding fragment thereof, of any one of claims 2-8, comprising:
a heavy chain variable region comprising a CDR1 provided by SEQ ID NO: 503, a CDR2 provided by SEQ ID NO: 504, and a CDR3 provided by SEQ ID NO: 505; and
a light chain variable region comprising a CDR1 provided by SEQ ID NO: 499, a CDR2 provided by SEQ ID NO: 500, and a CDR3 provided by SEQ ID NO: 501.

37. The anti-CLL-1 antibody, or antigen-binding fragment thereof, of any one of claims 2-8, comprising:
a heavy chain variable region comprising a CDR1 provided by SEQ ID NO: 511, a CDR2 provided by SEQ ID NO: 512, and a CDR3 provided by SEQ ID NO: 513; and
a light chain variable region comprising a CDR1 provided by SEQ ID NO: 507, a CDR2 provided by SEQ ID NO: 508, and a CDR3 provided by SEQ ID NO: 509.

38. The anti-CLL-1 antibody, or antigen-binding fragment thereof, of any one of claims 2-8, comprising:
a heavy chain variable region comprising a CDR1 provided by SEQ ID NO: 519, a CDR2 provided by SEQ ID NO: 520, and a CDR3 provided by SEQ ID NO: 521; and
a light chain variable region comprising a CDR1 provided by SEQ ID NO: 515, a CDR2 provided by SEQ ID NO: 516, and a CDR3 provided by SEQ ID NO: 517.

39. The anti-CLL-1 antibody, or antigen-binding fragment thereof, of any one of claims 2-8, comprising:
a heavy chain variable region comprising a CDR1 provided by SEQ ID NO: 527, a CDR2 provided by SEQ ID NO: 528, and a CDR3 provided by SEQ ID NO: 529; and
a light chain variable region comprising a CDR1 provided by SEQ ID NO: 523, a CDR2 provided by SEQ ID NO: 524, and a CDR3 provided by SEQ ID NO: 525.

40. The anti-CLL-1 antibody, or antigen-binding fragment thereof, of any one of claims 2-8, comprising:
a heavy chain variable region comprising a CDR1 provided by SEQ ID NO: 535, a CDR2 provided by SEQ ID NO: 536, and a CDR3 provided by SEQ ID NO: 537; and
a light chain variable region comprising a CDR1 provided by SEQ ID NO: 531, a CDR2 provided by SEQ ID NO: 532, and a CDR3 provided by SEQ ID NO: 533.

41. The anti-CLL-1 antibody, or antigen-binding fragment thereof, of any one of claims 2-8, comprising:
a heavy chain variable region comprising a CDR1 provided by SEQ ID NO: 543, a CDR2 provided by SEQ ID NO: 544, and a CDR3 provided by SEQ ID NO: 545; and
a light chain variable region comprising a CDR1 provided by SEQ ID NO: 539, a CDR2 provided by SEQ ID NO: 540, and a CDR3 provided by SEQ ID NO: 541.

42. The anti-CLL-1 antibody, or antigen-binding fragment thereof, of any one of claims 2-8, comprising:
a heavy chain variable region comprising a CDR1 provided by SEQ ID NO: 551, a CDR2 provided by SEQ ID NO: 552, and a CDR3 provided by SEQ ID NO: 553; and
a light chain variable region comprising a CDR1 provided by SEQ ID NO: 547, a CDR2 provided by SEQ ID NO: 548, and a CDR3 provided by SEQ ID NO: 549.

43. The anti-CLL-1 antibody, or antigen-binding fragment thereof, of any one of claims 2-8, comprising:
a heavy chain variable region comprising a CDR1 provided by SEQ ID NO: 559, a CDR2 provided by SEQ ID NO: 560, and a CDR3 provided by SEQ ID NO: 561; and
a light chain variable region comprising a CDR1 provided by SEQ ID NO: 555, a CDR2 provided by SEQ ID NO: 556, and a CDR3 provided by SEQ ID NO: 557.

44. The anti-CLL-1 antibody, or antigen-binding fragment thereof, of any one of claims 2-8, comprising:
a heavy chain variable region comprising a CDR1 provided by SEQ ID NO: 567, a CDR2 provided by SEQ ID NO: 568, and a CDR3 provided by SEQ ID NO: 569; and
a light chain variable region comprising a CDR1 provided by SEQ ID NO: 563, a CDR2 provided by SEQ ID NO: 564, and a CDR3 provided by SEQ ID NO: 565.

45. The anti-CLL-1 antibody, or antigen-binding fragment thereof, of any one of claims 2-8, comprising:
a heavy chain variable region comprising a CDR1 provided by SEQ ID NO: 575, a CDR2 provided by SEQ ID NO: 576, and a CDR3 provided by SEQ ID NO: 577; and
a light chain variable region comprising a CDR1 provided by SEQ ID NO: 571, a CDR2 provided by SEQ ID NO: 572, and a CDR3 provided by SEQ ID NO: 573.

46. The anti-CLL-1 antibody, or antigen-binding fragment thereof, of any one of claims 2-8, comprising:
a heavy chain variable region comprising a CDR1 provided by SEQ ID NO: 583, a CDR2 provided by SEQ ID NO: 584, and a CDR3 provided by SEQ ID NO: 585; and
a light chain variable region comprising a CDR1 provided by SEQ ID NO: 579, a CDR2 provided by SEQ ID NO: 580, and a CDR3 provided by SEQ ID NO: 581.

47. The anti-CLL-1 antibody, or antigen-binding fragment thereof, of any one of claims 2-8, comprising:
a heavy chain variable region comprising a CDR1 provided by SEQ ID NO: 591, a CDR2 provided by SEQ ID NO: 592, and a CDR3 provided by SEQ ID NO: 593; and
a light chain variable region comprising a CDR1 provided by SEQ ID NO: 594, a CDR2 provided by SEQ ID NO: 595, and a CDR3 provided by SEQ ID NO: 596.

48. The anti-CLL-1 antibody, or antigen-binding fragment thereof, of any one of claims 2-8, comprising:
a heavy chain variable region comprising a CDR1 provided by SEQ ID NO: 599, a CDR2 provided by SEQ ID NO: 600, and a CDR3 provided by SEQ ID NO: 601; and
a light chain variable region comprising a CDR1 provided by SEQ ID NO: 595, a CDR2 provided by SEQ ID NO: 596, and a CDR3 provided by SEQ ID NO: 597.

49. The anti-CLL-1 antibody, or antigen-binding fragment thereof, of any one of claims 2-8, comprising:
a heavy chain variable region comprising a CDR1 provided by SEQ ID NO: 607, a CDR2 provided by SEQ ID NO: 608, and a CDR3 provided by SEQ ID NO: 609; and
a light chain variable region comprising a CDR1 provided by SEQ ID NO: 603, a CDR2 provided by SEQ ID NO: 604, and a CDR3 provided by SEQ ID NO: 605.

50. The anti-CLL-1 antibody, or antigen-binding fragment thereof, of any one of claims 2-8, comprising:
a heavy chain variable region comprising a CDR1 provided by SEQ ID NO: 615, a CDR2 provided by SEQ ID NO: 616, and a CDR3 provided by SEQ ID NO: 617; and
a light chain variable region comprising a CDR1 provided by SEQ ID NO: 611, a CDR2 provided by SEQ ID NO: 612, and a CDR3 provided by SEQ ID NO: 613.

51. The anti-CLL-1 antibody, or antigen-binding fragment thereof, of any one of claims 2-8, comprising:
a heavy chain variable region comprising a CDR1 provided by SEQ ID NO: 623, a CDR2 provided by SEQ ID NO: 624, and a CDR3 provided by SEQ ID NO: 625; and
a light chain variable region comprising a CDR1 provided by SEQ ID NO: 619, a CDR2 provided by SEQ ID NO: 620, and a CDR3 provided by SEQ ID NO: 621.

52. The anti-CLL-1 antibody, or antigen-binding fragment thereof, of any one of claims 2-8, comprising:
a heavy chain variable region comprising a CDR1 provided by SEQ ID NO: 631, a CDR2 provided by SEQ ID NO: 632, and a CDR3 provided by SEQ ID NO: 633; and
a light chain variable region comprising a CDR1 provided by SEQ ID NO: 627, a CDR2 provided by SEQ ID NO: 628, and a CDR3 provided by SEQ ID NO: 629.

53. The anti-CLL-1 antibody, or antigen-binding fragment thereof, of any one of claims 2-8, comprising:
a heavy chain variable region comprising a CDR1 provided by SEQ ID NO: 639, a CDR2 provided by SEQ ID NO: 640, and a CDR3 provided by SEQ ID NO: 641; and
a light chain variable region comprising a CDR1 provided by SEQ ID NO: 635, a CDR2 provided by SEQ ID NO: 636, and a CDR3 provided by SEQ ID NO: 637.

54. The anti-CLL-1 antibody, or antigen-binding fragment thereof, of any one of claims 2-8, comprising:
a heavy chain variable region comprising a CDR1 provided by SEQ ID NO: 647, a CDR2 provided by SEQ ID NO: 648, and a CDR3 provided by SEQ ID NO: 649; and
a light chain variable region comprising a CDR1 provided by SEQ ID NO: 643, a CDR2 provided by SEQ ID NO: 644, and a CDR3 provided by SEQ ID NO: 645.

55. The anti-CLL-1 antibody, or antigen-binding fragment thereof, of any one of claims 2-8, comprising:
a heavy chain variable region comprising a CDR1 provided by SEQ ID NO: 655, a CDR2 provided by SEQ ID NO: 656, and a CDR3 provided by SEQ ID NO: 657; and
a light chain variable region comprising a CDR1 provided by SEQ ID NO: 651, a CDR2 provided by SEQ ID NO: 652, and a CDR3 provided by SEQ ID NO: 653.

56. The anti-CLL-1 antibody, or antigen-binding fragment thereof, of any one of claims 2-8, comprising:
a heavy chain variable region comprising a CDR1 provided by SEQ ID NO: 663, a CDR2 provided by SEQ ID NO: 664, and a CDR3 provided by SEQ ID NO: 665; and
a light chain variable region comprising a CDR1 provided by SEQ ID NO: 659, a CDR2 provided by SEQ ID NO: 660, and a CDR3 provided by SEQ ID NO: 661.

57. The anti-CLL-1 antibody, or antigen-binding fragment thereof, of any one of claims 2-8, comprising:
a heavy chain variable region comprising a CDR1 provided by SEQ ID NO: 671, a CDR2 provided by SEQ ID NO: 672, and a CDR3 provided by SEQ ID NO: 673; and
a light chain variable region comprising a CDR1 provided by SEQ ID NO: 667, a CDR2 provided by SEQ ID NO: 668, and a CDR3 provided by SEQ ID NO: 669.

58. The anti-CLL-1 antibody, or antigen-binding fragment thereof, of any one of claims 2-8, comprising:
a heavy chain variable region comprising a CDR1 provided by SEQ ID NO: 679, a CDR2 provided by SEQ ID NO: 680, and a CDR3 provided by SEQ ID NO: 681; and
a light chain variable region comprising a CDR1 provided by SEQ ID NO: 675, a CDR2 provided by SEQ ID NO: 676, and a CDR3 provided by SEQ ID NO: 677.

59. The anti-CLL-1 antibody, or antigen-binding fragment thereof, of any one of claims 2-8, comprising:
a heavy chain variable region comprising a CDR1 provided by SEQ ID NO: 687, a CDR2 provided by SEQ ID NO: 688, and a CDR3 provided by SEQ ID NO: 689; and
a light chain variable region comprising a CDR1 provided by SEQ ID NO: 683, a CDR2 provided by SEQ ID NO: 684, and a CDR3 provided by SEQ ID NO: 685.

60. The anti-CLL-1 antibody, or antigen-binding fragment thereof, of any one of claims 2-8, comprising:
a heavy chain variable region comprising a CDR1 provided by SEQ ID NO: 695, a CDR2 provided by SEQ ID NO: 696, and a CDR3 provided by SEQ ID NO: 697; and
a light chain variable region comprising a CDR1 provided by SEQ ID NO: 691, a CDR2 provided by SEQ ID NO: 692, and a CDR3 provided by SEQ ID NO: 693.

61. The anti-CLL-1 antibody, or antigen-binding fragment thereof, of any one of claims 2-8, comprising:
a heavy chain variable region comprising a CDR1 provided by SEQ ID NO: 703, a CDR2 provided by SEQ ID NO: 704, and a CDR3 provided by SEQ ID NO: 705; and
a light chain variable region comprising a CDR1 provided by SEQ ID NO: 699, a CDR2 provided by SEQ ID NO: 700, and a CDR3 provided by SEQ ID NO: 701.

62. The anti-CLL-1 antibody, or antigen-binding fragment thereof, of any one of claims 2-8, comprising:
a heavy chain variable region comprising a CDR1 provided by SEQ ID NO: 711, a CDR2 provided by SEQ ID NO: 712, and a CDR3 provided by SEQ ID NO: 713; and
a light chain variable region comprising a CDR1 provided by SEQ ID NO: 707, a CDR2 provided by SEQ ID NO: 708, and a CDR3 provided by SEQ ID NO: 709.

63. The anti-CLL-1 antibody, or antigen-binding fragment thereof, of any one of claims 2-8, comprising:
a heavy chain variable region comprising a CDR1 provided by SEQ ID NO: 823, a CDR2 provided by SEQ ID NO: 824, and a CDR3 provided by SEQ ID NO: 825; and
a light chain variable region comprising a CDR1 provided by SEQ ID NO: 820, a CDR2 provided by SEQ ID NO: 821, and a CDR3 provided by SEQ ID NO: 822.

64. The anti-CLL-1 antibody, or antigen-binding fragment thereof, of any one of claims 2-8, comprising:
a heavy chain variable region comprising a CDR1 provided by SEQ ID NO: 841, a CDR2 provided by SEQ ID NO: 842, and a CDR3 provided by SEQ ID NO: 843; and
a light chain variable region comprising a CDR1 provided by SEQ ID NO: 838, a CDR2 provided by SEQ ID NO: 839, and a CDR3 provided by SEQ ID NO: 840.

65. The anti-CLL-1 antibody, or antigen-binding fragment thereof, of any one of claims 2-8, comprising:
a heavy chain variable region comprising a CDR1 provided by SEQ ID NO: 852, a CDR2 provided by SEQ ID NO: 853, and a CDR3 provided by SEQ ID NO: 854; and
a light chain variable region comprising a CDR1 provided by SEQ ID NO: 850, a CDR2 provided by SEQ ID NO: 839, and a CDR3 provided by SEQ ID NO: 851.

66. The anti-CLL-1 antibody, or antigen-binding fragment thereof, of any one of claims 2-8, comprising:
a heavy chain variable region comprising a CDR1 provided by SEQ ID NO: 914, a CDR2 provided by SEQ ID NO: 915, and a CDR3 provided by SEQ ID NO: 916; and
a light chain variable region comprising a CDR1 provided by SEQ ID NO: 911, a CDR2 provided by SEQ ID NO: 912, and a CDR3 provided by SEQ ID NO: 913.

67. The anti-CLL-1 antibody, or antigen-binding fragment thereof, of any one of claims 2-8, comprising a CDR1 provided by SEQ ID NO: 715, a CDR2 provided by SEQ ID NO: 716, and a CDR3 provided by SEQ ID NO: 717.

68. The anti-CLL-1 antibody, or antigen-binding fragment thereof, of any one of claims 2-8, comprising a CDR1 provided by SEQ ID NO: 719, a CDR2 provided by SEQ ID NO: 720, and a CDR3 provided by SEQ ID NO: 721.

69. The anti-CLL-1 antibody, or antigen-binding fragment thereof, of any one of claims 2-8, comprising a CDR1 provided by SEQ ID NO: 723, a CDR2 provided by SEQ ID NO: 724, and a CDR3 provided by SEQ ID NO: 725.

70. The anti-CLL-1 antibody, or antigen-binding fragment thereof, of any one of claims 2-8, comprising a CDR1 provided by SEQ ID NO: 727, a CDR2 provided by SEQ ID NO: 728, and a CDR3 provided by SEQ ID NO: 729.

71. The anti-CLL-1 antibody, or antigen-binding fragment thereof, of any one of claims 2-8, comprising a CDR1 provided by SEQ ID NO: 731, a CDR2 provided by SEQ ID NO: 732, and a CDR3 provided by SEQ ID NO: 733.

72. The anti-CLL-1 antibody, or antigen-binding fragment thereof, of any one of claims 2-8, comprising a CDR1 provided by SEQ ID NO: 735, a CDR2 provided by SEQ ID NO: 736, and a CDR3 provided by SEQ ID NO: 737.

73. The anti-CLL-1 antibody, or antigen-binding fragment thereof, of any one of claims 2-8, comprising a CDR1 provided by SEQ ID NO: 739, a CDR2 provided by SEQ ID NO: 740, and a CDR3 provided by SEQ ID NO: 741.

74. The anti-CLL-1 antibody, or antigen-binding fragment thereof, of any one of claims 2-8, comprising a CDR1 provided by SEQ ID NO: 743, a CDR2 provided by SEQ ID NO: 744, and a CDR3 provided by SEQ ID NO: 745.

75. The anti-CLL-1 antibody, or antigen-binding fragment thereof, of any one of claims 2-8, comprising a CDR1 provided by SEQ ID NO: 747, a CDR2 provided by SEQ ID NO: 748, and a CDR3 provided by SEQ ID NO: 749.

76. The anti-CLL-1 antibody, or antigen-binding fragment thereof, of any one of claims 2-8, comprising a CDR1 provided by SEQ ID NO: 751, a CDR2 provided by SEQ ID NO: 752, and a CDR3 provided by SEQ ID NO: 753.

77. The anti-CLL-1 antibody, or antigen-binding fragment thereof, of any one of claims 2-8, comprising a CDR1 provided by SEQ ID NO: 755, a CDR2 provided by SEQ ID NO: 756, and a CDR3 provided by SEQ ID NO: 757.

78. The anti-CLL-1 antibody, or antigen-binding fragment thereof, of any one of claims 2-8, comprising a CDR1 provided by SEQ ID NO: 759, a CDR2 provided by SEQ ID NO: 760, and a CDR3 provided by SEQ ID NO: 761.

79. The anti-CLL-1 antibody, or antigen-binding fragment thereof, of any one of claims 2-8, comprising a CDR1 provided by SEQ ID NO: 763, a CDR2 provided by SEQ ID NO: 764, and a CDR3 provided by SEQ ID NO: 765.

80. The anti-CLL-1 antibody, or antigen-binding fragment thereof, of any one of claims 2-8, comprising a CDR1 provided by SEQ ID NO: 767, a CDR2 provided by SEQ ID NO: 768, and a CDR3 provided by SEQ ID NO: 769.

81. The anti-CLL-1 antibody, or antigen-binding fragment thereof, of any one of claims 2-8, comprising a CDR1 provided by SEQ ID NO: 770, a CDR2 provided by SEQ ID NO: 771, and a CDR3 provided by SEQ ID NO: 772.

82. The anti-CLL-1 antibody, or antigen-binding fragment thereof, of any one of claims 2-8, comprising a CDR1 provided by SEQ ID NO: 775, a CDR2 provided by SEQ ID NO: 776, and a CDR3 provided by SEQ ID NO: 777.

83. The anti-CLL-1 antibody, or antigen-binding fragment thereof, of any one of claims 2-8, comprising a CDR1 provided by SEQ ID NO: 780, a CDR2 provided by SEQ ID NO: 781, and a CDR3 provided by SEQ ID NO: 782.

84. The anti-CLL-1 antibody, or antigen-binding fragment thereof, of any one of claims 2-8, comprising a CDR1 provided by SEQ ID NO: 785, a CDR2 provided by SEQ ID NO: 786, and a CDR3 provided by SEQ ID NO: 777.

85. The anti-CLL-1 antibody, or antigen-binding fragment thereof, of any one of claims 2-8, comprising a CDR1 provided by SEQ ID NO: 789, a CDR2 provided by SEQ ID NO: 790, and a CDR3 provided by SEQ ID NO: 791.

86. The anti-CLL-1 antibody, or antigen-binding fragment thereof, of any one of claims 2-8, comprising a CDR1 provided by SEQ ID NO: 794, a CDR2 provided by SEQ ID NO: 795, and a CDR3 provided by SEQ ID NO: 796.

87. The anti-CLL-1 antibody, or antigen-binding fragment thereof, of any one of claims 2-8, comprising a CDR1 provided by SEQ ID NO: 785, a CDR2 provided by SEQ ID NO: 799, and a CDR3 provided by SEQ ID NO: 777.

88. The anti-CLL-1 antibody, or antigen-binding fragment thereof, of any one of claims 2-8, comprising a CDR1 provided by SEQ ID NO: 785, a CDR2 provided by SEQ ID NO: 802, and a CDR3 provided by SEQ ID NO: 777.

89. The anti-CLL-1 antibody, or antigen-binding fragment thereof, of any one of claims 2-8, comprising a CDR1 provided by SEQ ID NO: 789, a CDR2 provided by SEQ ID NO: 805, and a CDR3 provided by SEQ ID NO: 806.

90. The anti-CLL-1 antibody, or antigen-binding fragment thereof, of any one of claims 2-8, comprising a CDR1 provided by SEQ ID NO: 901, a CDR2 provided by SEQ ID NO: 902, and a CDR3 provided by SEQ ID NO: 903.

91. The anti-CLL-1 antibody, or antigen-binding fragment thereof, of any one of claims 2-8, comprising a CDR1 provided by SEQ ID NO: 906, a CDR2 provided by SEQ ID NO: 907, and a CDR3 provided by SEQ ID NO: 908.

92. An anti-CLL-1 antibody, or antigen-binding fragment thereof, comprising a VHH comprising
a CDR1 provided by SEQ ID NO: 5, 19, 33, 47, 61, 75, 89, 103, 117, 131, 145, 159, 170, 175, 180, 185, 190, 195, 200, 205, 210, 215, 220, 225, 230, 235, 240, 245, 250, 255, 260, 265, 271, 276, 281, 286, 291, 299, 313, 327, 341, 355,423, 431, 439, 447, 455, 463, 471, 479, 487, 495, 503, 511, 519, 527, 535, 543, 551, 559, 567, 575, 583, 591, 599, 607, 615, 623, 631, 639, 647, 655, 663, 671, 679, 687, 695, 703, 711, 715, 719, 723, 727, 731, 735, 739, 743, 747, 751, 755, 759, 763, 767, 770, 775, 780, 785, 789, 794, 823, 841, 852, 896, 901, 906, or 914;
a CDR2 provided by SEQ ID NO: 6, 20, 34, 48, 62, 76, 90, 104, 118, 132, 146, 160, 171, 176, 181, 186, 191, 196, 201, 206, 211, 216, 221, 226, 231, 236, 241, 246, 251, 256, 261, 266, 272, 277, 282, 287, 292, 300, 314, 328, 342,356, 424, 432, 440, 448, 456, 464, 472, 480, 488, 496, 504, 512, 520, 528, 536, 544, 552, 560, 568, 576, 584, 592, 600, 608, 616, 624, 632, 640, 648, 656, 664, 672, 680, 688, 696, 704, 712, 716, 720, 724, 728, 732, 736, 740, 744, 748, 752, 756, 760, 764, 768, 771, 776, 781, 786, 790, 795, 799, 802, 805, 824, 842, 853, 897, 902, 907, or 915; and
a CDR3 provided by SEQ ID NO: 7, 21, 35, 49, 63, 77, 91, 105, 119, 133, 147, 161, 172, 177, 182, 187, 192, 197, 202, 207, 212, 217, 222, 227, 232, 237, 242, 247, 252, 257, 262, 267, 273, 278, 283, 288, 293, 301, 315, 329, 343, 357, 425, 433, 441, 449, 457, 465, 473, 481, 489, 497, 505, 513, 521, 529, 537, 545, 553, 561, 569, 577, 585, 593, 601, 609, 617, 625, 633, 641, 649, 657, 665, 673, 681, 689, 697, 705, 713, 717, 721, 725, 729, 733, 737, 741, 745, 749, 753, 757, 761, 765, 769, 772, 777, 782, 791, 796, 806, 825, 843, 854, 898, 903, 908, or 916.

93. The anti-CLL-1 antibody, or antigen-binding fragment thereof, of claim 92, wherein the VHH comprises a CDR1 provided by SEQ ID NO: 170, a CDR2 provided by SEQ ID NO: 171, and a CDR3 provided by SEQ ID NO: 172.

94. The anti-CLL-1 antibody, or antigen-binding fragment thereof, of claim 92, wherein the VHH comprises a CDR1 provided by SEQ ID NO: 175, a CDR2 provided by SEQ ID NO: 176, and a CDR3 provided by SEQ ID NO: 177.

95. The anti-CLL-1 antibody, or antigen-binding fragment thereof, of claim 92, wherein the VHH comprises a CDR1 provided by SEQ ID NO: 180, a CDR2 provided by SEQ ID NO: 181, and a CDR3 provided by SEQ ID NO: 182.

96. The anti-CLL-1 antibody, or antigen-binding fragment thereof, of claim 92, wherein the VHH comprises a CDR1 provided by SEQ ID NO: 185, a CDR2 provided by SEQ ID NO: 186, and a CDR3 provided by SEQ ID NO: 187.

97. The anti-CLL-1 antibody, or antigen-binding fragment thereof, of claim 92, wherein the VHH comprises a CDR1 provided by SEQ ID NO: 190, a CDR2 provided by SEQ ID NO: 191, and a CDR3 provided by SEQ ID NO: 192.

98. The anti-CLL-1 antibody, or antigen-binding fragment thereof, of claim 92, wherein the VHH comprises a CDR1 provided by SEQ ID NO: 195, a CDR2 provided by SEQ ID NO: 196, and a CDR3 provided by SEQ ID NO: 197.

99. The anti-CLL-1 antibody, or antigen-binding fragment thereof, of claim 92, wherein the VHH comprises a CDR1 provided by SEQ ID NO: 200, a CDR2 provided by SEQ ID NO: 201, and a CDR3 provided by SEQ ID NO: 202.

100. The anti-CLL-1 antibody, or antigen-binding fragment thereof, of claim 92, wherein the VHH comprises a CDR1 provided by SEQ ID NO: 205, a CDR2 provided by SEQ ID NO: 206, and a CDR3 provided by SEQ ID NO: 207.

101. The anti-CLL-1 antibody, or antigen-binding fragment thereof, of claim 92, wherein the VHH comprises a CDR1 provided by SEQ ID NO: 210, a CDR2 provided by SEQ ID NO: 211, and a CDR3 provided by SEQ ID NO: 212.

102. The anti-CLL-1 antibody, or antigen-binding fragment thereof, of claim 92, wherein the VHH comprises a CDR1 provided by SEQ ID NO: 215, a CDR2 provided by SEQ ID NO: 216, and a CDR3 provided by SEQ ID NO: 217.

103. The anti-CLL-1 antibody, or antigen-binding fragment thereof, of claim 92, wherein the VHH comprises a CDR1 provided by SEQ ID NO: 220, a CDR2 provided by SEQ ID NO: 221, and a CDR3 provided by SEQ ID NO: 222.

104. The anti-CLL-1 antibody, or antigen-binding fragment thereof, of claim 92, wherein the VHH comprises a CDR1 provided by SEQ ID NO: 225, a CDR2 provided by SEQ ID NO: 226, and a CDR3 provided by SEQ ID NO: 227.

105. The anti-CLL-1 antibody, or antigen-binding fragment thereof, of claim 92, wherein the VHH comprises a CDR1 provided by SEQ ID NO: 230, a CDR2 provided by SEQ ID NO: 231, and a CDR3 provided by SEQ ID NO: 232.

106. The anti-CLL-1 antibody, or antigen-binding fragment thereof, of claim 92, wherein the VHH comprises a CDR1 provided by SEQ ID NO: 235, a CDR2 provided by SEQ ID NO: 236, and a CDR3 provided by SEQ ID NO: 237.

107. The anti-CLL-1 antibody, or antigen-binding fragment thereof, of claim 92, wherein the VHH comprises a CDR1 provided by SEQ ID NO: 240, a CDR2 provided by SEQ ID NO: 241, and a CDR3 provided by SEQ ID NO: 242.

108. The anti-CLL-1 antibody, or antigen-binding fragment thereof, of claim 92, wherein the VHH comprises a CDR1 provided by SEQ ID NO: 245, a CDR2 provided by SEQ ID NO: 246, and a CDR3 provided by SEQ ID NO: 247.

109. The anti-CLL-1 antibody, or antigen-binding fragment thereof, of claim 92, wherein the VHH comprises a CDR1 provided by SEQ ID NO: 250, a CDR2 provided by SEQ ID NO: 251, and a CDR3 provided by SEQ ID NO: 252.

110. The anti-CLL-1 antibody, or antigen-binding fragment thereof, of claim 92, wherein the VHH comprises a CDR1 provided by SEQ ID NO: 255, a CDR2 provided by SEQ ID NO: 256, and a CDR3 provided by SEQ ID NO: 257.

111. The anti-CLL-1 antibody, or antigen-binding fragment thereof, of claim 92, wherein the VHH comprises a CDR1 provided by SEQ ID NO: 260, a CDR2 provided by SEQ ID NO: 261, and a CDR3 provided by SEQ ID NO: 262.

112. The anti-CLL-1 antibody, or antigen-binding fragment thereof, of claim 92, wherein the VHH comprises a CDR1 provided by SEQ ID NO: 265, a CDR2 provided by SEQ ID NO: 266, and a CDR3 provided by SEQ ID NO: 267.

113. The anti-CLL-1 antibody, or antigen-binding fragment thereof, of claim 92, wherein the VHH comprises a CDR1 provided by SEQ ID NO: 271, a CDR2 provided by SEQ ID NO: 272, and a CDR3 provided by SEQ ID NO: 273.

114. The anti-CLL-1 antibody, or antigen-binding fragment thereof, of claim 92, wherein the VHH comprises a CDR1 provided by SEQ ID NO: 276, a CDR2 provided by SEQ ID NO: 277, and a CDR3 provided by SEQ ID NO: 278.

115. The anti-CLL-1 antibody, or antigen-binding fragment thereof, of claim 92, wherein the VHH comprises a CDR1 provided by SEQ ID NO: 281, a CDR2 provided by SEQ ID NO: 282, and a CDR3 provided by SEQ ID NO: 283.

116. The anti-CLL-1 antibody, or antigen-binding fragment thereof, of claim 92, wherein the VHH comprises a CDR1 provided by SEQ ID NO: 286, a CDR2 provided by SEQ ID NO: 287, and a CDR3 provided by SEQ ID NO: 288.

117. The anti-CLL-1 antibody, or antigen-binding fragment thereof, of claim 92, wherein the VHH comprises a CDR1 provided by SEQ ID NO: 291, a CDR2 provided by SEQ ID NO: 292, and a CDR3 provided by SEQ ID NO: 293.

118. The anti-CLL-1 antibody, or antigen-binding fragment thereof, of claim 92, wherein the VHH comprises a CDR1 provided by SEQ ID NO: 715, a CDR2 provided by SEQ ID NO: 716, and a CDR3 provided by SEQ ID NO: 717.

119. The anti-CLL-1 antibody, or antigen-binding fragment thereof, of claim 92, wherein the VHH comprises a CDR1 provided by SEQ ID NO: 719, a CDR2 provided by SEQ ID NO: 720, and a CDR3 provided by SEQ ID NO: 721.

120. The anti-CLL-1 antibody, or antigen-binding fragment thereof, of claim 92, wherein the VHH comprises a CDR1 provided by SEQ ID NO: 723, a CDR2 provided by SEQ ID NO: 724, and a CDR3 provided by SEQ ID NO: 725.

121. The anti-CLL-1 antibody, or antigen-binding fragment thereof, of claim 92, wherein the VHH comprises a CDR1 provided by SEQ ID NO: 727, a CDR2 provided by SEQ ID NO: 728, and a CDR3 provided by SEQ ID NO: 729.

122. The anti-CLL-1 antibody, or antigen-binding fragment thereof, of claim 92, wherein the VHH comprises a CDR1 provided by SEQ ID NO: 731, a CDR2 provided by SEQ ID NO: 732, and a CDR3 provided by SEQ ID NO: 733.

123. The anti-CLL-1 antibody, or antigen-binding fragment thereof, of claim 92, wherein the VHH comprises a CDR1 provided by SEQ ID NO: 735, a CDR2 provided by SEQ ID NO: 736, and a CDR3 provided by SEQ ID NO: 737.

124. The anti-CLL-1 antibody, or antigen-binding fragment thereof, of claim 92, wherein the VHH comprises a CDR1 provided by SEQ ID NO: 739, a CDR2 provided by SEQ ID NO: 740, and a CDR3 provided by SEQ ID NO: 741.

125. The anti-CLL-1 antibody, or antigen-binding fragment thereof, of claim 92, wherein the VHH comprises a CDR1 provided by SEQ ID NO: 743, a CDR2 provided by SEQ ID NO: 744, and a CDR3 provided by SEQ ID NO: 745.

126. The anti-CLL-1 antibody, or antigen-binding fragment thereof, of claim 92, wherein the VHH comprises a CDR1 provided by SEQ ID NO: 747, a CDR2 provided by SEQ ID NO: 748, and a CDR3 provided by SEQ ID NO: 749.

127. The anti-CLL-1 antibody, or antigen-binding fragment thereof, of claim 92, wherein the VHH comprises a CDR1 provided by SEQ ID NO: 751, a CDR2 provided by SEQ ID NO: 752, and a CDR3 provided by SEQ ID NO: 753.

128. The anti-CLL-1 antibody, or antigen-binding fragment thereof, of claim 92, wherein the VHH comprises a CDR1 provided by SEQ ID NO: 755, a CDR2 provided by SEQ ID NO: 756, and a CDR3 provided by SEQ ID NO: 757.

129. The anti-CLL-1 antibody, or antigen-binding fragment thereof, of claim 92, wherein the VHH comprises a CDR1 provided by SEQ ID NO: 759, a CDR2 provided by SEQ ID NO: 760, and a CDR3 provided by SEQ ID NO: 761.

130. The anti-CLL-1 antibody, or antigen-binding fragment thereof, of claim 92, wherein the VHH comprises a CDR1 provided by SEQ ID NO: 763, a CDR2 provided by SEQ ID NO: 764, and a CDR3 provided by SEQ ID NO: 765.

131. The anti-CLL-1 antibody, or antigen-binding fragment thereof, of claim 92, wherein the VHH comprises a CDR1 provided by SEQ ID NO: 767, a CDR2 provided by SEQ ID NO: 768, and a CDR3 provided by SEQ ID NO: 769.

132. The anti-CLL-1 antibody, or antigen-binding fragment thereof, of claim 92, wherein the VHH comprises a CDR1 provided by SEQ ID NO: 770, a CDR2 provided by SEQ ID NO: 771, and a CDR3 provided by SEQ ID NO: 772.

133. The anti-CLL-1 antibody, or antigen-binding fragment thereof, of claim 92, wherein the VHH comprises a CDR1 provided by SEQ ID NO: 775, a CDR2 provided by SEQ ID NO: 776, and a CDR3 provided by SEQ ID NO: 777.

134. The anti-CLL-1 antibody, or antigen-binding fragment thereof, of claim 92, wherein the VHH comprises a CDR1 provided by SEQ ID NO: 780, a CDR2 provided by SEQ ID NO: 781, and a CDR3 provided by SEQ ID NO: 782.

135. The anti-CLL-1 antibody, or antigen-binding fragment thereof, of claim 92, wherein the VHH comprises a CDR1 provided by SEQ ID NO: 785, a CDR2 provided by SEQ ID NO: 786, and a CDR3 provided by SEQ ID NO: 777.

136. The anti-CLL-1 antibody, or antigen-binding fragment thereof, of claim 92, wherein the VHH comprises a CDR1 provided by SEQ ID NO: 789, a CDR2 provided by SEQ ID NO: 790, and a CDR3 provided by SEQ ID NO: 791.

137. The anti-CLL-1 antibody, or antigen-binding fragment thereof, of claim 92, wherein the VHH comprises a CDR1 provided by SEQ ID NO: 794, a CDR2 provided by SEQ ID NO: 795, and a CDR3 provided by SEQ ID NO: 796.

138. The anti-CLL-1 antibody, or antigen-binding fragment thereof, of claim 92, wherein the VHH comprises a CDR1 provided by SEQ ID NO: 785, a CDR2 provided by SEQ ID NO: 799, and a CDR3 provided by SEQ ID NO: 777.

139. The anti-CLL-1 antibody, or antigen-binding fragment thereof, of claim 92, wherein the VHH comprises a CDR1 provided by SEQ ID NO: 785, a CDR2 provided by SEQ ID NO: 802, and a CDR3 provided by SEQ ID NO: 777.

140. The anti-CLL-1 antibody, or antigen-binding fragment thereof, of claim 92, wherein the VHH comprises a CDR1 provided by SEQ ID NO: 789, a CDR2 provided by SEQ ID NO: 805, and a CDR3 provided by SEQ ID NO: 806.

141. The anti-CLL-1 antibody, or antigen-binding fragment thereof, of claim 92, wherein the VHH comprises a CDR1 provided by SEQ ID NO: 823, a CDR2 provided by SEQ ID NO: 824, and a CDR3 provided by SEQ ID NO: 825.

142. The anti-CLL-1 antibody, or antigen-binding fragment thereof, of claim 92, wherein the VHH comprises a CDR1 provided by SEQ ID NO: 823, a CDR2 provided by SEQ ID NO: 824, and a CDR3 provided by SEQ ID NO: 825.

143. The anti-CLL-1 antibody, or antigen-binding fragment thereof, of claim 92, wherein the VHH comprises a CDR1 provided by SEQ ID NO: 841, a CDR2 provided by SEQ ID NO: 842, and a CDR3 provided by SEQ ID NO: 843.

144. The anti-CLL-1 antibody, or antigen-binding fragment thereof, of claim 92, wherein the VHH comprises a CDR1 provided by SEQ ID NO: 852, a CDR2 provided by SEQ ID NO: 853, and a CDR3 provided by SEQ ID NO: 854.

145. The anti-CLL-1 antibody, or antigen-binding fragment thereof, of claim 9, wherein the VHH comprises a CDR1 provided by SEQ ID NO: 896, a CDR2 provided by SEQ ID NO: 897, and a CDR3 provided by SEQ ID NO: 898.

146. The anti-CLL-1 antibody, or antigen-binding fragment thereof, of claim 9, wherein the VHH comprises a CDR1 provided by SEQ ID NO: 901, a CDR2 provided by SEQ ID NO: 902, and a CDR3 provided by SEQ ID NO: 903.

147. The anti-CLL-1 antibody, or antigen-binding fragment thereof, of claim 9, wherein the VHH comprises a CDR1 provided by SEQ ID NO: 906, a CDR2 provided by SEQ ID NO: 907, and a CDR3 provided by SEQ ID NO: 908.

148. The anti-CLL-1 antibody, or antigen-binding fragment thereof, of claim 9, wherein the VHH comprises a CDR1 provided by SEQ ID NO: 914, a CDR2 provided by SEQ ID NO: 915, and a CDR3 provided by SEQ ID NO: 916.

149. An anti-CLL-1 antibody, or antigen-binding fragment thereof, comprising a VHH comprising an amino acid sequence of SEQ ID NOs: 174, 179, 184, 189, 194, 199, 204, 209, 214, 219, 224, 229, 234, 239, 244, 249, 254, 259, 264, 269, 275, 280, 285, 290, 295, 714, 718, 722, 726, 730, 734, 738, 742, 746, 750, 754, 758, 762, 766, 774, 779, 784, 788, 793, 798, 801, 804, 808, 829, 835, 847, 858, 900, 905, 910, or 920.

150. An anti-CLL-1 antibody, or antigen-binding fragment thereof, comprising a VHH comprising a CDR sequence of SEQ ID NOs: 5-7, 19-21, 33-35, 47-49, 61-63, 75-77, 89-91, 103-105, 117-119, 131-133, 145-147, 159-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 299-301, 313-315, 327-329, 341-343, 355-357, 423-425, 431-433, 439-441, 447-449, 455-457, 463-465, 471-473, 479-481, 487-489, 495-497, 503-505, 511-513, 519-521, 527-529, 535-537, 543-545, 551-553, 559-561, 567-569, 575-577, 583-585, 591-593, 599-601, 607-609, 615-617, 623-625, 631-633, 639-641, 647-649, 655-657, 663-665, 671-673, 679-681, 687-689, 695-697, 703-705, 711-713, 715-717, 719-821, 723-725, 727-729, 731-733, 735-737, 739-741, 743-745, 747-749, 751-753, 755-757, 759-761, 763-765, 767-772, 775-777, 780-782, 785-786, 789-791, 794-796, 799, 802, 805-806, 823-825, 841-843, 852-854, 896-898, 901-903, 906-908, or 914-916.

151. An anti-CLL-1 antibody, or antigen-binding fragment thereof, comprising a VHH comprising a CDR1, a CDR2, and a CDR3 of SEQ ID NOs: 5-7, 19-21, 33-35, 47-49, 61-63, 75-77, 89-91, 103-105, 117-119, 131-133, 145-147, 159-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 299-301, 313-315, 327-329, 341-343, 355-357, 423-425, 431-433, 439-441, 447-449, 455-457, 463-465, 471-473, 479-481, 487-489, 495-497, 503-505, 511-513, 519-521, 527-529, 535-537, 543-545, 551-553, 559-561, 567-569, 575-577, 583-585, 591-593, 599-601, 607-609, 615-617, 623-625, 631-633, 639-641, 647-649, 655-657, 663-665, 671-673, 679-681, 687-689, 695-697, 703-705, 711-713, 715-717, 719-821, 723-725, 727-729, 731-733, 735-737, 739-741, 743-745, 747-749, 751-753, 755-757, 759-761, 763-765, 767-772, 775-777, 780-782, 785-786, 789-791, 794-796, 799, 802, 805-806, 823-825, 841-843, 852-854, 896-898, 901-903, 906-908, or 914-916.

152. An anti-CLL-1 antibody, or antigen-binding fragment thereof, comprising a VHH comprising at least one CDR (e.g., CDR1, CDR2, and/or CDR3) of SEQ ID NOs: 5-7, 19-21, 33-35, 47-49, 61-63, 75-77, 89-91, 103-105, 117-119, 131-133, 145-147, 159-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 299-301, 313-315, 327-329, 341-343, 355-357, 423-425, 431-433, 439-441, 447-449, 455-457, 463-465, 471-473, 479-481, 487-489, 495-497, 503-505, 511-513, 519-521, 527-529, 535-537, 543-545, 551-553, 559-561, 567-569, 575-577, 583-585, 591-593, 599-601, 607-609, 615-617, 623-625, 631-633, 639-641, 647-649, 655-657, 663-665, 671-673, 679-681, 687-689, 695-697, 703-705, 711-713, 715-717, 719-821, 723-725, 727-729, 731-733, 735-737, 739-741, 743-745, 747-749, 751-753, 755-757, 759-761, 763-765, 767-772, 775-777, 780-782, 785-786, 789-791, 794-796, 799, 802, 805-806, 823-825, 841-843, 852-854, 896-898, 901-903, 906-908, or 914-916.

153. An anti-CLL-1 antibody, or antigen-binding fragment thereof, comprising a VHH comprising at least one CDR that is at least 85%, 90%, 91%, 92%, 93%, 94%, 95%, 96%, 97%, 98%, 99%, or 100% identical to a CDR (e.g., CDR1, CDR2, and/or CDR3) of SEQ ID NOs: 5-7, 19-21, 33-35, 47-49, 61-63, 75-77, 89-91, 103-105, 117-119, 131-133, 145-147, 159-161, 170-172, 175-177, 180-182, 185-187, 190-192, 195-197, 200-202, 205-207, 210-212, 215-217, 220-222, 225-227, 230-232, 235-237, 240-242, 245-247, 250-252, 255-257, 260-262, 265-267, 271-273, 276-278, 281-283, 286-288, 291-293, 299-301, 313-315, 327-329, 341-343, 355-357, 423-425, 431-433, 439-441, 447-449, 455-457, 463-465, 471-473, 479-481, 487-489, 495-497, 503-505, 511-513, 519-521, 527-529, 535-537, 543-545, 551-553, 559-561, 567-569, 575-577, 583-585, 591-593, 599-601, 607-609, 615-617, 623-625, 631-633, 639-641, 647-649, 655-657, 663-665, 671-673, 679-681, 687-689, 695-697, 703-705, 711-713, 715-717, 719-821, 723-725, 727-729, 731-733, 735-737, 739-741, 743-745, 747-749, 751-753, 755-757, 759-761, 763-765, 767-772, 775-777, 780-782, 785-786, 789-791, 794-796, 799, 802, 805-806, 823-825, 841-843, 852-854, 896-898, 901-903, 906-908, or 914-916.

154. The anti-CLL-1 antibody, or antigen-binding fragment thereof, of any one of claims 1-153, wherein the antibody, or antigen-binding fragment thereof, is a monoclonal antibody, or antigen-binding fragment thereof.

155. The anti-CLL-1 antibody, or antigen-binding fragment thereof, of any one of claims 1-154, wherein the antibody, or antigen-binding fragment thereof, is a humanized antibody, or antigen-binding fragment thereof.

156. An anti-CLL-1 antibody, or antigen-binding fragment thereof, that competes with the antibody, or antigen-binding fragment thereof, of any one of claims 1-155.

157. The anti-CLL-1 antibody, or antigen-binding fragment thereof, of any one of claims 1-156, wherein the antibody, or antigen-binding fragment thereof, comprises a CH2 constant domain and a CH3 constant domain.

158. The anti-CLL-1 antibody, or antigen-binding fragment thereof, of claim 157, wherein the antibody, or antigen-binding fragment thereof, comprises an amino acid sequence of SEQ ID NO: 15, 29, 43, 57, 71, 85, 99, 113, 127, 141, 155, 169, 174, 179, 184, 189, 194, 199, 204, 209, 214, 219, 224, 229, 234, 239, 244, 249, 254, 259, 264, 269, 275, 280, 285, 290, 295, 309, 323, 337, 351, 365, 418, 422, 426, 430, 434, 438, 442, 446, 450, 454, 458, 462, 466, 470, 474, 478, 482, 486, 490, 494, 498, 502, 506, 510, 514, 518, 522, 526, 530, 534, 538, 542, 546, 550, 554, 558, 562, 566, 570, 574, 578, 582, 586, 590, 584, 598, 602, 606, 610, 614, 618, 622, 626, 630, 634, 638, 642, 646, 650, 654, 658, 662, 666, 670, 674, 678, 682, 686, 690, 694, 698, 702, 706, 710, 714, 718, 722, 726, 730, 734, 738, 742, 746, 750, 754, 758, 762, 766, 774, 779, 784, 788, 793, 798, 801, 804, 808, 817, 829, 831, 835, 837, 847, 849, 858, 860, 893, 900, 905, 910, 920.

159. The anti-CLL-1 antibody, or antigen-binding fragment thereof, of any one of claims 1-158, wherein the antibody, or antigen-binding fragment thereof, is a heavy chain antibody.

160. The anti-CLL-1 antibody, or antigen-binding fragment thereof, of any one of claims 1-159, wherein the antibody, or antigen-binding fragment thereof, is a camelid antibody.

161. The anti-CLL-1 antibody, or antigen-binding fragment thereof, of any one of claims 1-159, wherein the antibody, or antigen-binding fragment thereof, is a llama antibody.

162. The anti-CLL-1 antibody, or antigen-binding fragment thereof, of any one of claims 1-159, wherein the antibody, or antigen-binding fragment thereof, is an alpaca antibody.

163. The anti-CLL-1 antibody, or antigen-binding fragment thereof, of any one of claims 1-158, wherein said antibody is of the IgG1-, IgG2-, IgG3 or IgG4-type.

164. A chimeric antigen receptor comprising any of the antibody, or antigen-binding fragment thereof, of any one of claims 1-163.

165. A cell expressing the chimeric antigen receptor of claim 164.

166. The cell of claim 165, wherein the cell is an immune effector cell.

167. The cell of claim 165 or 166, wherein the cell is a lymphocyte.

168. The cell of any one of claims 165-167, wherein the cell is a T-cell.

169. The cell of any one of claims 165-167, wherein the cell is a Natural Killer (NK) cell.

170. A pharmaceutical composition comprising at least one antibody of any one of claims 1-163, chimeric antigen receptor of claim 164, or cell of any one of claims 165-169, and a pharmaceutically acceptable excipient.

171. A nucleic acid, comprising a nucleic acid sequence encoding an antibody, or antigen-binding fragment thereof, of any one of claims 1-163, or the chimeric antigen receptor of claim 164.

172. The nucleic acid of claim 172, wherein the nucleic acid comprises a sequence that is 95-99% identical to the sequence of any one of SEQ ID NOs: 8, 10, 12, 14, 22, 24, 26, 28, 36, 38, 40, 42, 50, 52, 54, 56, 64, 66, 68, 70, 78, 80, 82, 84, 92, 94, 96, 98, 106, 108, 110, 112, 120, 122, 124, 126, 134, 136, 138, 140, 148, 150, 152, 154, 162, 164, 166, 168, 173, 178, 183, 188, 193, 198, 203, 208, 213, 218, 223, 228, 233, 238, 243, 248, 253, 258, 263, 268, 274, 279, 284, 289, 294, 302, 304, 306, 308, 316, 318, 320, 322, 330, 332, 334, 336, 344, 346, 348, 350, 358, 360, 362, 364, 773, 778, 783, 787, 792, 797, 800, 803, 807, 826, 828, 830, 832, 834, 836, 844, 846, 848, 855, 857, 859, 904, 909, 917, 919, or 921.

173. The nucleic acid of claim 172, wherein the nucleic acid comprises the sequence of any one of SEQ ID NOs: 8, 10, 12, 14, 22, 24, 26, 28, 36, 38, 40, 42, 50, 52, 54, 56, 64, 66, 68, 70, 78, 80, 82, 84, 92, 94, 96, 98, 106, 108, 110, 112, 120, 122, 124, 126, 134, 136, 138, 140, 148, 150, 152, 154, 162, 164, 166, 168, 173, 178, 183, 188, 193, 198, 203, 208, 213, 218, 223, 228, 233, 238, 243, 248, 253, 258, 263, 268, 274, 279, 284, 289, 294, 302, 304, 306, 308, 316, 318, 320, 322, 330, 332, 334, 336, 344, 346, 348, 350, 358, 360, 362, 364, 773, 778, 783, 787, 792, 797, 800, 803, 807, 826, 828, 830, 832, 834, 836, 844, 846, 848, 855, 857, 859, 904, 909, 917, 919, or 921.

174. A vector comprising the nucleic acid of any one of claims 171-173.

175. A cell comprising the nucleic acid of any one of claims 171-173 or the vector of claim 174.

176. The cell of claim 175, wherein the cell is an immune cell.

177. The cell of claim 176, wherein the immune cell is a T cell, a Natural Killer (NK) cell, a cytotoxic T lymphocyte (CTL), or a regulatory T cell.

178. A method of producing an antibody, or antigen-binding fragment thereof, comprising culturing the cell of any one of claims 165-169 or 175-177 under conditions suitable for expression of the antibody or antigen-binding fragment thereof.

179. A method of treating a CLL-1-associated disease or disorder, the method comprising administering to a subject in need thereof an effective amount of the antibody, or antigen-binding fragment thereof, of any one of claims 1-163, or the cell of any one of claims 165-169 or 175-177.

180. The method of claim 179, wherein the CLL-1-associated disease or disorder is myelodysplastic syndrome (MDS) or acute myeloid leukemia (AML).

181. The method of claim 179 or 180, further comprising administering to the subject an effective amount of a chemotherapeutic agent or an oncolytic therapeutic agent.

182. A method of treating a subject having or at risk of a neoplastic disease or malignancy of the blood that is associated with CLL-1 expression, the method comprising administering to the subject a therapeutically effective amount of the antibody, or antigen-binding fragment thereof, of any one of claims 1-163, or the cell of any one of claims 165-169 or 175-177.

183. The method of claim 182, wherein the neoplastic disease or malignancy of the blood that is associated with CLL-1 expression is myelodysplastic syndrome (MDS) or acute myeloid leukemia (AML).

184. The method of claim 182 or 183, further comprising administering to the subject an effective amount of a chemotherapeutic agent or an oncolytic therapeutic agent.

185. A chimeric antigen receptor (CAR), wherein the CAR comprises a CLL-1 binding domain, a transmembrane domain, and an intracellular signaling domain,
wherein the CLL-1 binding domain comprises a heavy chain variable region and/or a light chain variable region;
wherein the transmembrane domain comprises a transmembrane domain of a protein selected from CD8α or CD28; and
wherein the intracellular signaling domain comprises a signaling domain of CD3ζ.

186. The CAR of claim 185, wherein the heavy chain variable region and the light chain variable region are joined by a linker.

187. The CAR of claim 185 or 186, wherein the CLL-1 binding domain comprises at least one of a single-chain variable fragment (scFv), a VH fragment, a VHH fragment, or a VL fragment.

188. The CAR of any one of claims 185-187, wherein the CLL-1 binding domain is connected to the transmembrane domain by a hinge region.

189. The CAR of claim 188, wherein the hinge region comprises a hinge region of a protein selected from CD8α, IgG4, or CD28.

190. The CAR of any one of claims 185-189, further comprising one or more co-stimulatory domains.

191. The CAR of claim 190, wherein the one more co-stimulatory domains comprises a signaling domain of 4-1BB and/or CD28.

192. The CAR of any one of claims 185-191, wherein the CLL-1 binding domain comprises the anti-CLL-1 antibody, or antigen-binding fragment thereof, or any one of claims 1-164.

193. An nucleic acid encoding the CAR of any one of claims 185-192.

194. The nucleic acid of claim 193, wherein the isolated nucleic acid comprises 95-99% sequence identity to the sequence selected from any one of SEQ ID NOs: 8, 10, 12, 14, 22, 24, 26, 28, 36, 38, 40, 42, 50, 52, 54, 56, 64, 66, 68, 70, 78, 80, 82, 84, 92, 94, 96, 98, 106, 108, 110, 112, 120, 122, 124, 126, 134, 136, 138, 140, 148, 150, 152, 154, 162, 164, 166, 168, 173, 178, 183, 188, 193, 198, 203, 208, 213, 218, 223, 228, 233, 238, 243, 248, 253, 258, 263, 268, 274, 279, 284, 289, 294, 302, 304, 306, 308, 316, 318, 320, 322, 330, 332, 334, 336, 344, 346, 348, 350, 358, 360, 362, 364, 773, 778, 783, 787, 792, 797, 800, 803, 807, 826, 828, 830, 832, 834, 836, 844, 846, 848, 855, 857, 859, 904, 909, 917, 919, or 921.

195. The nucleic acid of claim 194, wherein the isolated nucleic acid comprises the sequence selected from any one of SEQ ID NOs: 8, 10, 12, 14, 22, 24, 26, 28, 36, 38, 40, 42, 50, 52, 54, 56, 64, 66, 68, 70, 78, 80, 82, 84, 92, 94, 96, 98, 106, 108, 110, 112, 120, 122, 124, 126, 134, 136, 138, 140, 148, 150, 152, 154, 162, 164, 166, 168, 173, 178, 183, 188, 193, 198, 203, 208, 213, 218, 223, 228, 233, 238, 243, 248, 253, 258, 263, 268, 274, 279, 284, 289, 294, 302, 304, 306, 308, 316, 318, 320, 322, 330, 332, 334, 336, 344, 346, 348, 350, 358, 360, 362, 364, 773, 778, 783, 787, 792, 797, 800, 803, 807, 826, 828, 830, 832, 834, 836, 844, 846, 848, 855, 857, 859, 904, 909, 917, 919, 921.

196. The nucleic acid of any one of claims 193-195, wherein the CAR sequence is operably linked to a promoter sequence.

197. The nucleic acid of claim 196, wherein the promoter sequence is an SFFV promoter, an EF1α promoter, a tEF1α promoter, a hPGK promoter, a SFFV promoter, or a MND promoter.

198. An expression vector comprising the nucleic acid molecule encoding a CAR of any one of claims 193-197.

199. The expression vector of claim 198, wherein the vector is a DNA vector, an RNA vector, a plasmid, a lentivirus vector, an adenoviral vector or a retrovirus vector.

200. An immune effector cell comprising the CAR of any one of claims 185-192 or the nucleic acid molecule of any one of claims 193-197.

201. The immune effector cell of claim 202, wherein the cell is selected from the group consisting of a T cell, a Natural Killer (NK) cell, a cytotoxic T lymphocyte (CTL), a regulatory T cell, a human embryonic stem cell, and a pluripotent stem cell from which lymphoid cells may be differentiated.

202. A population of cells comprising at least one immune effector cell of claim 200 or claim 201.

203. A pharmaceutical composition comprising the population of cells of claim 202 and a pharmaceutically acceptable carrier.

204. A method of treating a hematopoietic malignancy, comprising administering to a subject in need thereof an effective amount of the CAR of any one of claims 185-192, the immune effector cell of claim 200 or 201, or population of cells of claim 202, or pharmaceutical composition of claim 203.

205. The method of claim 204, where the method further comprises administering a population of hematopoietic cells, wherein the hematopoietic cells are genetically-engineered such that the gene encoding CLL-1 that is targeted by the antigen-binding domain is engineered to reduce or eliminate the expression of CLL-1.

206. The method of claim 205, wherein the immune cells, the hematopoietic cells, or both, are allogeneic or autologous.

207. The method of any one of claims 205 or 206, wherein the hematopoietic cells are hematopoietic stem cells.

208. The method of claim 207, wherein the hematopoietic stem cells are from bone marrow cells or peripheral blood mononuclear cells (PBMCs).

209. The method of claim 207 or claim 208, wherein the hematopoietic stem cells are CD34+/CLL-1−.

210. The method of any one of claims 205-209, wherein the hematopoietic cells are prepared by editing the endogenous gene coding for CLL-1 to reduce or eliminate the expression of CLL-1.

211. The method of claim 210, wherein the endogenous gene is edited by CRISPR-Cas9.

212. The method of any one of claims 204-211, wherein the subject has or has been diagnosed with a hematopoietic malignancy or pre-malignancy characterized by the expression of CLL-1 on malignant cells or pre-malignant cells.

213. The method of any one of claims 204-212, wherein the subject has Hodgkin's lymphoma, non-Hodgkin's lymphoma, leukemia, or multiple myeloma.

214. The method of claim 213, wherein the leukemia is acute myeloid leukemia, myelodysplastic syndrome, chronic myelogenous leukemia, acute lymphoblastic leukemia, or chronic lymphoblastic leukemia.


SEQUENCE LISTING (928 sequences)

SEQ ID NO: 1 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 11 aa, synthetic construct
GGNNIGSKSVH

SEQ ID NO: 3 - protein, 7 aa, synthetic construct
DDSDRPS

SEQ ID NO: 4 - protein, 11 aa, synthetic construct
QVWDSSSDHPV

SEQ ID NO: 5 - protein, 7 aa, synthetic construct
GGTFSSY

SEQ ID NO: 6 - protein, 6 aa, synthetic construct
IPILGI

SEQ ID NO: 7 - protein, 18 aa, synthetic construct
AREKDDILTGAYYYGMDV

SEQ ID NO: 8 - DNA, 324 bp, synthetic construct
  source 1..324: mol_type = other DNA
tcctatgtgctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatccggtattcggcggaggaacccagctgatcatttta

SEQ ID NO: 9 - protein, 108 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHPVFGGGTQLIIL

SEQ ID NO: 10 - DNA, 45 bp, synthetic construct
  source 1..45: mol_type = other DNA
ggtggaggcggttccggaggcggtggcagcggcggtggcggttcg

SEQ ID NO: 11 - protein, 15 aa, synthetic construct
GGGGSGGGGSGGGGS

SEQ ID NO: 12 - DNA, 375 bp, synthetic construct
  source 1..375: mol_type = other DNA
caggtccagctggtacagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacgaatccacgagcacagcctacatggagctgagcagcctgagatctgaggacacggccgtgtattactgtgcgagagagaaggacgatattttgactggggcctactactacggtatggacgtctggggccaagggaccacggtcaccgtctcctca

SEQ ID NO: 13 - protein, 125 aa, synthetic construct
QVQLVQSGAEVKKPGSSVKVSCKASGGTFSSYAISWVRQAPGQGLEWMGRIIPILGIANYAQKFQGRVTITADESTSTAYMELSSLRSEDTAVYYCAREKDDILTGAYYYGMDVWGQGTTVTVSS

SEQ ID NO: 14 - DNA, 744 bp, synthetic construct
  source 1..744: mol_type = other DNA
tcctatgtgctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatccggtattcggcggaggaacccagctgatcattttaggtggaggcggttccggaggcggtggcagcggcggtggcggttcgcaggtccagctggtacagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacgaatccacgagcacagcctacatggagctgagcagcctgagatctgaggacacggccgtgtattactgtgcgagagagaaggacgatattttgactggggcctactactacggtatggacgtctggggccaagggaccacggtcaccgtctcctca

SEQ ID NO: 15 - protein, 248 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHPVFGGGTQLIILGGGGSGGGGSGGGGSQVQLVQSGAEVKKPGSSVKVSCKASGGTFSSYAISWVRQAPGQGLEWMGRIIPILGIANYAQKFQGRVTITADESTSTAYMELSSLRSEDTAVYYCAREKDDILTGAYYYGMDVWGQGTTVTVSS

SEQ ID NO: 16 - protein, 11 aa, synthetic construct
GGNNIGSKNVH

SEQ ID NO: 17 - protein, 7 aa, synthetic construct
RDSNRPS

SEQ ID NO: 18 - protein, 9 aa, synthetic construct
QVWDSNTYV

SEQ ID NO: 19 - protein, 7 aa, synthetic construct
GFTFSDY

SEQ ID NO: 20 - protein, 6 aa, synthetic construct
SSSGST

SEQ ID NO: 21 - protein, 13 aa, synthetic construct
ARQSAEIVGAFDH

SEQ ID NO: 22 - DNA, 318 bp, synthetic construct
  source 1..318: mol_type = other DNA
cagtctgtgttgacgcagccactctcagtgtcggtggccctgggacagacggccaggattacctgtgggggaaacaacattggaagtaaaaatgtgcactggtaccagcagaaggcaggccaggcccctgtgctggtcatccaaagagatagcaaccggccctctgggatccctgagcgattctctggctccaattcggggaacacggccaccctgaccatcagtagagcccaagccggggatgaggctgactatttctgtcaggtgtgggacagcaacacttatgtcttcggaactgggaccaagctgaccgtccta

SEQ ID NO: 23 - protein, 106 aa, synthetic construct
QSVLTQPLSVSVALGQTARITCGGNNIGSKNVHWYQQKAGQAPVLVIQRDSNRPSGIPERFSGSNSGNTATLTISRAQAGDEADYFCQVWDSNTYVFGTGTKLTVL

SEQ ID NO: 24 - DNA, 45 bp, synthetic construct; identical to SEQ ID NO: 10
  source 1..45: mol_type = other DNA

SEQ ID NO: 25 - protein, 15 aa, synthetic construct
GGGGSGGGGSGGGGS

SEQ ID NO: 26 - DNA, 360 bp, synthetic construct
  source 1..360: mol_type = other DNA
gaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagacgaagacacggctgtgtattactgtgcgagacaatccgcagagatagtgggagcttttgaccactggggccaggggacaatggtcaccgtctcttca

SEQ ID NO: 27 - protein, 120 aa, synthetic construct
EVQLVESGGGLVKPGGSLRLSCAASGFTFSDYYMSWIRQAPGKGLEWVSYISSSGSTIYYADSVKGRFTISRDNAKNSLYLQMNSLRDEDTAVYYCARQSAEIVGAFDHWGQGTMVTVSS

SEQ ID NO: 28 - DNA, 723 bp, synthetic construct
  source 1..723: mol_type = other DNA
cagtctgtgttgacgcagccactctcagtgtcggtggccctgggacagacggccaggattacctgtgggggaaacaacattggaagtaaaaatgtgcactggtaccagcagaaggcaggccaggcccctgtgctggtcatccaaagagatagcaaccggccctctgggatccctgagcgattctctggctccaattcggggaacacggccaccctgaccatcagtagagcccaagccggggatgaggctgactatttctgtcaggtgtgggacagcaacacttatgtcttcggaactgggaccaagctgaccgtcctaggtggaggcggttccggaggcggtggcagcggcggtggcggttcggaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagacgaagacacggctgtgtattactgtgcgagacaatccgcagagatagtgggagcttttgaccactggggccaggggacaatggtcaccgtctcttca

SEQ ID NO: 29 - protein, 241 aa, synthetic construct
QSVLTQPLSVSVALGQTARITCGGNNIGSKNVHWYQQKAGQAPVLVIQRDSNRPSGIPERFSGSNSGNTATLTISRAQAGDEADYFCQVWDSNTYVFGTGTKLTVLGGGGSGGGGSGGGGSEVQLVESGGGLVKPGGSLRLSCAASGFTFSDYYMSWIRQAPGKGLEWVSYISSSGSTIYYADSVKGRFTISRDNAKNSLYLQMNSLRDEDTAVYYCARQSAEIVGAFDHWGQGTMVTVSS

SEQ ID NO: 30 - protein, 14 aa, synthetic construct
TGTSSDVGGYNYVS

SEQ ID NO: 31 - protein, 7 aa, synthetic construct
EVGKRPS

SEQ ID NO: 32 - protein, 10 aa, synthetic construct
SSYTSSSTRV

SEQ ID NO: 33 - protein, 7 aa, synthetic construct
GYTLTEL

SEQ ID NO: 34 - protein, 6 aa, synthetic construct
DPEDGE

SEQ ID NO: 35 - protein, 8 aa, synthetic construct
ATDPHMDV

SEQ ID NO: 36 - DNA, 330 bp, synthetic construct
  source 1..330: mol_type = other DNA
cagtctgccctgactcagcctccctccgcgtccgggtctcctggacagtcagtcaccatctcctgcactggaaccagcagtgacgttggtggttataactatgtctcctggtaccaacagcacccaggcaaagcccccaaactcatgatttatgaggtcggtaagcggccctcaggggtccctgatcgcttctctggctccaagtctggcaacacggcctccctgaccgtctctgggctccaggctgaggatgaggctgattattactgcagctcatatacaagcagcagcacgcgggtgttcggcggagggaccaaggtcaccgtccta

SEQ ID NO: 37 - protein, 110 aa, synthetic construct
QSALTQPPSASGSPGQSVTISCTGTSSDVGGYNYVSWYQQHPGKAPKLMIYEVGKRPSGVPDRFSGSKSGNTASLTVSGLQAEDEADYYCSSYTSSSTRVFGGGTKVTVL

SEQ ID NO: 38 - DNA, 45 bp, synthetic construct; identical to SEQ ID NO: 10
  source 1..45: mol_type = other DNA

SEQ ID NO: 39 - protein, 15 aa, synthetic construct
GGGGSGGGGSGGGGS

SEQ ID NO: 40 - DNA, 345 bp, synthetic construct
  source 1..345: mol_type = other DNA
gaggtccagctggtacagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatcctgaagatggtgaaacaatctacgcacagaagtttcagggcagagtcaccatgaccgaggacacatctacagacacagcctacatggagctgagcagcctgagatctgaggacacggccgtgtattattgtgcaacagatccccacatggacgtctggggccaagggacaatggtcaccgtctcttca

SEQ ID NO: 41 - protein, 115 aa, synthetic construct
EVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDPHMDVWGQGTMVTVSS

SEQ ID NO: 42 - DNA, 720 bp, synthetic construct
  source 1..720: mol_type = other DNA
cagtctgccctgactcagcctccctccgcgtccgggtctcctggacagtcagtcaccatctcctgcactggaaccagcagtgacgttggtggttataactatgtctcctggtaccaacagcacccaggcaaagcccccaaactcatgatttatgaggtcggtaagcggccctcaggggtccctgatcgcttctctggctccaagtctggcaacacggcctccctgaccgtctctgggctccaggctgaggatgaggctgattattactgcagctcatatacaagcagcagcacgcgggtgttcggcggagggaccaaggtcaccgtcctaggtggaggcggttccggaggcggtggcagcggcggtggcggttcggaggtccagctggtacagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatcctgaagatggtgaaacaatctacgcacagaagtttcagggcagagtcaccatgaccgaggacacatctacagacacagcctacatggagctgagcagcctgagatctgaggacacggccgtgtattattgtgcaacagatccccacatggacgtctggggccaagggacaatggtcaccgtctcttca

SEQ ID NO: 43 - protein, 240 aa, synthetic construct
QSALTQPPSASGSPGQSVTISCTGTSSDVGGYNYVSWYQQHPGKAPKLMIYEVGKRPSGVPDRFSGSKSGNTASLTVSGLQAEDEADYYCSSYTSSSTRVFGGGTKVTVLGGGGSGGGGSGGGGSEVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDPHMDVWGQGTMVTVSS

SEQ ID NO: 44 - protein, 13 aa, synthetic construct
SGSSPNIGKNYVS

SEQ ID NO: 45 - protein, 7 aa, synthetic construct
DNNKRPS

SEQ ID NO: 46 - protein, 11 aa, synthetic construct
GTWDSSLSGYV

SEQ ID NO: 47 - protein, 7 aa, synthetic construct
GFTFSSY

SEQ ID NO: 48 - protein, 6 aa, synthetic construct
SHDGSN

SEQ ID NO: 49 - protein, 15 aa, synthetic construct
AREGQLTTSWDFFDY

SEQ ID NO: 50 - DNA, 330 bp, synthetic construct
  source 1..330: mol_type = other DNA
cagtctgtcgtgacgcagccgccctcagtgtctgcggccccaggacagaaggtcaccatctcctgctctggaagcagccccaatattggcaaaaattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtggttatgtcttcggaactgggaccaaggtgaccgtccta

SEQ ID NO: 51 - protein, 110 aa, synthetic construct
QSVVTQPPSVSAAPGQKVTISCSGSSPNIGKNYVSWYQQLPGTAPKLLIYDNNKRPSGIPDRFSGSKSGTSATLGITGLQTGDEADYYCGTWDSSLSGYVFGTGTKVTVL

SEQ ID NO: 52 - DNA, 45 bp, synthetic construct; identical to SEQ ID NO: 10
  source 1..45: mol_type = other DNA

SEQ ID NO: 53 - protein, 15 aa, synthetic construct
GGGGSGGGGSGGGGS

SEQ ID NO: 54 - DNA, 366 bp, synthetic construct
  source 1..366: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcatttatatcacatgatggaagcaataaatactacgcagactccgtgaagggccgatccaccatctccagagacaattccaagagcacgctgtttctgcaaatgaacagcctgagagatgaggacacggctgtatatttctgtgcgagagaagggcagctcacaacttcttgggacttctttgactactggggccacggaaccctggtcactgtctcctca

SEQ ID NO: 55 - protein, 122 aa, synthetic construct
QVQLVESGGGVVQPGRSLRLSCAASGFTFSSYGMHWVRQAPGKGLEWVAFISHDGSNKYYADSVKGRSTISRDNSKSTLFLQMNSLRDEDTAVYFCAREGQLTTSWDFFDYWGHGTLVTVSS

SEQ ID NO: 56 - DNA, 741 bp, synthetic construct
  source 1..741: mol_type = other DNA
cagtctgtcgtgacgcagccgccctcagtgtctgcggccccaggacagaaggtcaccatctcctgctctggaagcagccccaatattggcaaaaattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtggttatgtcttcggaactgggaccaaggtgaccgtcctaggtggaggcggttccggaggcggtggcagcggcggtggcggttcgcaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcatttatatcacatgatggaagcaataaatactacgcagactccgtgaagggccgatccaccatctccagagacaattccaagagcacgctgtttctgcaaatgaacagcctgagagatgaggacacggctgtatatttctgtgcgagagaagggcagctcacaacttcttgggacttctttgactactggggccacggaaccctggtcactgtctcctca

SEQ ID NO: 57 - protein, 247 aa, synthetic construct
QSVVTQPPSVSAAPGQKVTISCSGSSPNIGKNYVSWYQQLPGTAPKLLIYDNNKRPSGIPDRFSGSKSGTSATLGITGLQTGDEADYYCGTWDSSLSGYVFGTGTKVTVLGGGGSGGGGSGGGGSQVQLVESGGGVVQPGRSLRLSCAASGFTFSSYGMHWVRQAPGKGLEWVAFISHDGSNKYYADSVKGRSTISRDNSKSTLFLQMNSLRDEDTAVYFCAREGQLTTSWDFFDYWGHGTLVTVSS

SEQ ID NO: 58 - protein, 11 aa, synthetic construct
RASQSISSYLN

SEQ ID NO: 59 - protein, 7 aa, synthetic construct
AASSLQS

SEQ ID NO: 60 - protein, 9 aa, synthetic construct
QQSYSTPWT

SEQ ID NO: 61 - protein, 7 aa, synthetic construct
GFTFSSY

SEQ ID NO: 62 - protein, 6 aa, synthetic construct
SHDGSN

SEQ ID NO: 63 - protein, 15 aa, synthetic construct
AREGQLTTSWDFFDY

SEQ ID NO: 64 - DNA, 321 bp, synthetic construct
  source 1..321: mol_type = other DNA
gacatccagttgacccagtctccatcctccctgtctgcatctgtaggagacagagtcaccatcacttgccgggcaagccagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgtggacgttcggccaagggaccaaggtggagatcaaa

SEQ ID NO: 65 - protein, 107 aa, synthetic construct
DIQLTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKVEIK

SEQ ID NO: 66 - DNA, 45 bp, synthetic construct; identical to SEQ ID NO: 10
  source 1..45: mol_type = other DNA

SEQ ID NO: 67 - protein, 15 aa, synthetic construct
GGGGSGGGGSGGGGS

SEQ ID NO: 68 - DNA, 366 bp, synthetic construct; identical to SEQ ID NO: 54
  source 1..366: mol_type = other DNA

SEQ ID NO: 69 - protein, 122 aa, synthetic construct; identical to SEQ ID NO: 55

SEQ ID NO: 70 - DNA, 732 bp, synthetic construct
  source 1..732: mol_type = other DNA
gacatccagttgacccagtctccatcctccctgtctgcatctgtaggagacagagtcaccatcacttgccgggcaagccagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgtggacgttcggccaagggaccaaggtggagatcaaaggtggaggcggttccggaggcggtggcagcggcggtggcggttcgcaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcatttatatcacatgatggaagcaataaatactacgcagactccgtgaagggccgatccaccatctccagagacaattccaagagcacgctgtttctgcaaatgaacagcctgagagatgaggacacggctgtatatttctgtgcgagagaagggcagctcacaacttcttgggacttctttgactactggggccacggaaccctggtcactgtctcctca

SEQ ID NO: 71 - protein, 244 aa, synthetic construct
DIQLTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKVEIKGGGGSGGGGSGGGGSQVQLVESGGGVVQPGRSLRLSCAASGFTFSSYGMHWVRQAPGKGLEWVAFISHDGSNKYYADSVKGRSTISRDNSKSTLFLQMNSLRDEDTAVYFCAREGQLTTSWDFFDYWGHGTLVTVSS

SEQ ID NO: 72 - protein, 13 aa, synthetic construct
SGGSSNIGKNYVS

SEQ ID NO: 73 - protein, 7 aa, synthetic construct
ENQHRPS

SEQ ID NO: 74 - protein, 11 aa, synthetic construct
ATWDDSLSAGV

SEQ ID NO: 75 - protein, 7 aa, synthetic construct
GYSFTSY

SEQ ID NO: 76 - protein, 6 aa, synthetic construct
YPGDSD

SEQ ID NO: 77 - protein, 18 aa, synthetic construct
ARLGGYSGYDSEVFYLDY

SEQ ID NO: 78 - DNA, 330 bp, synthetic construct
  source 1..330: mol_type = other DNA
cagtctgtgttgacgcagccgccctcagtgtctgcggccccaggacagaaggtcagcatcttttgctctggaggcagctccaacattgggaagaactacgtatcctggtaccagcagctcccaggaacagcccccaaactcctcatctatgaaaatcagcatcgaccctcagggattcctgaccgattctctggctccaagtctggcacgtcagccaccctgggcatcaccggactccagactggggacgaggccgattattactgcgcaacatgggatgacagcctgagtgctggggtgttcggcggagggaccgagctgaccgtccta

SEQ ID NO: 79 - protein, 110 aa, synthetic construct
QSVLTQPPSVSAAPGQKVSIFCSGGSSNIGKNYVSWYQQLPGTAPKLLIYENQHRPSGIPDRFSGSKSGTSATLGITGLQTGDEADYYCATWDDSLSAGVFGGGTELTVL

SEQ ID NO: 80 - DNA, 45 bp, synthetic construct; identical to SEQ ID NO: 10
  source 1..45: mol_type = other DNA

SEQ ID NO: 81 - protein, 15 aa, synthetic construct
GGGGSGGGGSGGGGS

SEQ ID NO: 82 - DNA, 369 bp, synthetic construct
  source 1..369: mol_type = other DNA
cagctggtgcagtctggagcagaggtgagaaagcccggggagtctctgaagatctcctgtaagggttctggatacagctttaccagctactggatcggctgggtgcgccagatgcccgggaaaggcctggagtggatggggttcatctatcctggtgactctgataccagatacagcccgtccttccaaggccaggtcaccatctcagccgacaagtccatcagcaccgcctacctgcagcggagcagcctgaaggcctcggacaccgccatgtattactgtgcgagactcggtggatatagtggctacgattcggaagtcttttacttagactactggggccagggaaccacggtcaccgtctcctca

SEQ ID NO: 83 - protein, 123 aa, synthetic construct
QLVQSGAEVRKPGESLKISCKGSGYSFTSYWIGWVRQMPGKGLEWMGFIYPGDSDTRYSPSFQGQVTISADKSISTAYLQRSSLKASDTAMYYCARLGGYSGYDSEVFYLDYWGQGTTVTVSS

SEQ ID NO: 84 - DNA, 744 bp, synthetic construct
  source 1..744: mol_type = other DNA
cagtctgtgttgacgcagccgccctcagtgtctgcggccccaggacagaaggtcagcatcttttgctctggaggcagctccaacattgggaagaactacgtatcctggtaccagcagctcccaggaacagcccccaaactcctcatctatgaaaatcagcatcgaccctcagggattcctgaccgattctctggctccaagtctggcacgtcagccaccctgggcatcaccggactccagactggggacgaggccgattattactgcgcaacatgggatgacagcctgagtgctggggtgttcggcggagggaccgagctgaccgtcctaggtggaggcggttccggaggcggtggcagcggcggtggcggttcgcagctggtgcagtctggagcagaggtgagaaagcccggggagtctctgaagatctcctgtaagggttctggatacagctttaccagctactggatcggctgggtgcgccagatgcccgggaaaggcctggagtggatggggttcatctatcctggtgactctgataccagatacagcccgtccttccaaggccaggtcaccatctcagccgacaagtccatcagcaccgcctacctgcagcggagcagcctgaaggcctcggacaccgccatgtattactgtgcgagactcggtggatatagtggctacgattcggaagtcttttacttagactactggggccagggaaccacggtcaccgtctcctca

SEQ ID NO: 85 - protein, 248 aa, synthetic construct
QSVLTQPPSVSAAPGQKVSIFCSGGSSNIGKNYVSWYQQLPGTAPKLLIYENQHRPSGIPDRFSGSKSGTSATLGITGLQTGDEADYYCATWDDSLSAGVFGGGTELTVLGGGGSGGGGSGGGGSQLVQSGAEVRKPGESLKISCKGSGYSFTSYWIGWVRQMPGKGLEWMGFIYPGDSDTRYSPSFQGQVTISADKSISTAYLQRSSLKASDTAMYYCARLGGYSGYDSEVFYLDYWGQGTTVTVSS

SEQ ID NO: 86 - protein, 14 aa, synthetic construct
TGTSSDVGGYNYVS

SEQ ID NO: 87 - protein, 7 aa, synthetic construct
EVGKRPS

SEQ ID NO: 88 - protein, 10 aa, synthetic construct
SSYTSSSTRV

SEQ ID NO: 89 - protein, 7 aa, synthetic construct
GYTLTEL

SEQ ID NO: 90 - protein, 6 aa, synthetic construct
DPEDGE

SEQ ID NO: 91 - protein, 8 aa, synthetic construct
ATDPHMDV

SEQ ID NO: 92 - DNA, 330 bp, synthetic construct; identical to SEQ ID NO: 36
  source 1..330: mol_type = other DNA

SEQ ID NO: 93 - protein, 110 aa, synthetic construct; identical to SEQ ID NO: 37

SEQ ID NO: 94 - DNA, 45 bp, synthetic construct; identical to SEQ ID NO: 10
  source 1..45: mol_type = other DNA

SEQ ID NO: 95 - protein, 15 aa, synthetic construct
GGGGSGGGGSGGGGS

SEQ ID NO: 96 - DNA, 345 bp, synthetic construct; identical to SEQ ID NO: 40
  source 1..345: mol_type = other DNA

SEQ ID NO: 97 - protein, 115 aa, synthetic construct; identical to SEQ ID NO: 41

SEQ ID NO: 98 - DNA, 720 bp, synthetic construct; identical to SEQ ID NO: 42
  source 1..720: mol_type = other DNA

SEQ ID NO: 99 - protein, 240 aa, synthetic construct; identical to SEQ ID NO: 43

SEQ ID NO: 100 - protein, 11 aa, synthetic construct
RASQSISSYLN

SEQ ID NO: 101 - protein, 7 aa, synthetic construct
AASSLQS

SEQ ID NO: 102 - protein, 9 aa, synthetic construct
QQSYSTPLT

SEQ ID NO: 103 - protein, 7 aa, synthetic construct
GGTFTSY

SEQ ID NO: 104 - protein, 6 aa, synthetic construct
SAYNGN

SEQ ID NO: 105 - protein, 11 aa, synthetic construct
ALPTADDAFDI

SEQ ID NO: 106 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggtgtcccatcaaggttcagtggcagtggatctgggacagatttcactctcaccatcagcagtctgcaacctgaagattttgcaacttactactgtcaacagagttacagtacccctctcactttcggcggagggaccaaggtggaaatcaaa

SEQ ID NO: 107 - protein, 107 aa, synthetic construct [sequence omitted]

SEQ ID NO: 108 - DNA, 45 bp, synthetic construct; identical to SEQ ID NO: 10
  source 1..45: mol_type = other DNA

SEQ ID NO: 109 - protein, 15 aa, synthetic construct
GGGGSGGGGSGGGGS

SEQ ID NO: 110 - DNA, 354 bp, synthetic construct
  source 1..354: mol_type = other DNA
caggtgcagctggtgcaatctggggctgaggtgaagaagcctgggtcctcggtgaaggtctcctgcaaggcttctggaggcaccttcaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagagccgaggacacggccgtgtattactgtgccttacctacggccgatgatgcttttgatatctggggccaagggacaacggtcaccgtctcctca

SEQ ID NO: 111 - protein, 118 aa, synthetic construct
QVQLVQSGAEVKKPGSSVKVSCKASGGTFTSYGISWVRQAPGQGLEWMGWISAYNGNTNYAQKLQGRVTMTTDTSTSTAYMELRSLRAEDTAVYYCALPTADDAFDIWGQGTTVTVSS

SEQ ID NO: 112 - DNA, 720 bp, synthetic construct
  source 1..720: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggtgtcccatcaaggttcagtggcagtggatctgggacagatttcactctcaccatcagcagtctgcaacctgaagattttgcaacttactactgtcaacagagttacagtacccctctcactttcggcggagggaccaaggtggaaatcaaaggtggaggcggttccggaggcggtggcagcggcggtggcggttcgcaggtgcagctggtgcaatctggggctgaggtgaagaagcctgggtcctcggtgaaggtctcctgcaaggcttctggaggcaccttcaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagagccgaggacacggccgtgtattactgtgccttacctacggccgatgatgcttttgatatctggggccaagggacaacggtcaccgtctcctca

SEQ ID NO: 113 - protein, 240 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSGGGGSGGGGSQVQLVQSGAEVKKPGSSVKVSCKASGGTFTSYGISWVRQAPGQGLEWMGWISAYNGNTNYAQKLQGRVTMTTDTSTSTAYMELRSLRAEDTAVYYCALPTADDAFDIWGQGTTVTVSS

SEQ ID NO: 114 - protein, 11 aa, synthetic construct
GGNNIGSKSVH

SEQ ID NO: 115 - protein, 7 aa, synthetic construct
DDSDRPS

SEQ ID NO: 116 - protein, 12 aa, synthetic construct
QVWDSGSDHPVV

SEQ ID NO: 117 - protein, 7 aa, synthetic construct
GFTFSSY

SEQ ID NO: 118 - protein, 6 aa, synthetic construct
GGSGGS

SEQ ID NO: 119 - protein, 7 aa, synthetic construct
ANIDGTF

SEQ ID NO: 120 - DNA, 327 bp, synthetic construct
  source 1..327: mol_type = other DNA
tcctatgagctgactcagccaccctcggtgtcagtggccccaggaaagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtggtagtgatcatcccgtggtatccggcggaggaacccagctgatcatttta

SEQ ID NO: 121 - protein, 109 aa, synthetic construct
SYELTQPPSVSVAPGKTARITCGGNNIGSKSVHWYQQKPGQAPVLVVYDDSDRPSGIPERFSGSNSGNTATLTISRVEAGDEADYYCQVWDSGSDHPVVSGGGTQLIIL

SEQ ID NO: 122 - DNA, 45 bp, synthetic construct; identical to SEQ ID NO: 10
  source 1..45: mol_type = other DNA

SEQ ID NO: 123 - protein, 15 aa, synthetic construct
GGGGSGGGGSGGGGS

SEQ ID NO: 124 - DNA, 342 bp, synthetic construct
  source 1..342: mol_type = other DNA
caggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattggtggtagtggtggtagcacatactacgcagactccgtgaagggccggttcaccatctccagagacaattccaagaacacgctgtatctgcaaatgaacagcctgagagccgaggacacggccgtatattactgtgcgaatatcgacgggaccttctggggccagggaaccacggtcaccgtctcctca

SEQ ID NO: 125 - protein, 114 aa, synthetic construct
QVQLVESGGGLVQPGGSLRLSCAASGFTFSSYAMSWVRQAPGKGLEWVSAIGGSGGSTYYADSVKGRFTISRDNSKNTLYLQMNSLRAEDTAVYYCANIDGTFWGQGTTVTVSS

SEQ ID NO: 126 - DNA, 714 bp, synthetic construct
  source 1..714: mol_type = other DNA
tcctatgagctgactcagccaccctcggtgtcagtggccccaggaaagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtggtagtgatcatcccgtggtatccggcggaggaacccagctgatcattttaggtggaggcggttccggaggcggtggcagcggcggtggcggttcgcaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattggtggtagtggtggtagcacatactacgcagactccgtgaagggccggttcaccatctccagagacaattccaagaacacgctgtatctgcaaatgaacagcctgagagccgaggacacggccgtatattactgtgcgaatatcgacgggaccttctggggccagggaaccacggtcaccgtctcctca

SEQ ID NO: 127 - protein, 238 aa, synthetic construct
SYELTQPPSVSVAPGKTARITCGGNNIGSKSVHWYQQKPGQAPVLVVYDDSDRPSGIPERFSGSNSGNTATLTISRVEAGDEADYYCQVWDSGSDHPVVSGGGTQLIILGGGGSGGGGSGGGGSQVQLVESGGGLVQPGGSLRLSCAASGFTFSSYAMSWVRQAPGKGLEWVSAIGGSGGSTYYADSVKGRFTISRDNSKNTLYLQMNSLRAEDTAVYYCANIDGTFWGQGTTVTVSS

SEQ ID NO: 128 - protein, 11 aa, synthetic construct
RASQGISSYLA

SEQ ID NO: 129 - protein, 7 aa, synthetic construct
SASSLQS

SEQ ID NO: 130 - protein, 8 aa, synthetic construct
QQSYNSPT

SEQ ID NO: 131 - protein, 7 aa, synthetic construct
GYTFTGY

SEQ ID NO: 132 - protein, 6 aa, synthetic construct
NPNSGN

SEQ ID NO: 133 - protein, 11 aa, synthetic construct
ARGDTGDFFDH

SEQ ID NO: 134 - DNA, 318 bp, synthetic construct
  source 1..318: mol_type = other DNA
gacatccagatgacccagtctccatccttcctgtctgcatctgtaggagacagagtcaccatcacttgccgggccagtcagggcattagcagttatttagcctggtatcagcaaaaacctgggaaagcccctaagctcctgatctattctgcatccagtttgcaaagtggggtcccatcaaggttcagtggcagtggatctgggacagatttcactctcaccatcagcagtctgcaacctgaagattttgcaacttactactgtcaacagagttacaattcccctacgttcggccaagggacacgactggagattaaa

SEQ ID NO: 135 - protein, 106 aa, synthetic construct
DIQMTQSPSFLSASVGDRVTITCRASQGISSYLAWYQQKPGKAPKLLIYSASSLQSGVPSRFSGSGSGTDFTLTISSLQPEDFATYYCQQSYNSPTFGQGTRLEIK

SEQ ID NO: 136 - DNA, 45 bp, synthetic construct; identical to SEQ ID NO: 10
  source 1..45: mol_type = other DNA

SEQ ID NO: 137 - protein, 15 aa, synthetic construct
GGGGSGGGGSGGGGS

SEQ ID NO: 138 - DNA, 354 bp, synthetic construct
  source 1..354: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatgaaccctaacagtggtaacacaggctatgcacagaagttccagggcagagtcaccatgaccaggaacacctccataagcacagcctacttggagctgaccagcctgacatctgacgacacggccgtctattactgtgccagaggtgacacaggcgacttctttgaccattggggccagggaaccctggtcaccgtctcctca

SEQ ID NO: 139 - protein, 118 aa, synthetic construct
QVQLVQSGAEVKKPGASVKVSCKASGYTFTGYYMHWVRQAPGQGLEWMGWMNPNSGNTGYAQKFQGRVTMTRNTSISTAYLELTSLTSDDTAVYYCARGDTGDFFDHWGQGTLVTVSS

SEQ ID NO: 140 - DNA, 717 bp, synthetic construct
  source 1..717: mol_type = other DNA
gacatccagatgacccagtctccatccttcctgtctgcatctgtaggagacagagtcaccatcacttgccgggccagtcagggcattagcagttatttagcctggtatcagcaaaaacctgggaaagcccctaagctcctgatctattctgcatccagtttgcaaagtggggtcccatcaaggttcagtggcagtggatctgggacagatttcactctcaccatcagcagtctgcaacctgaagattttgcaacttactactgtcaacagagttacaattcccctacgttcggccaagggacacgactggagattaaaggtggaggcggttccggaggcggtggcagcggcggtggcggttcgcaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatgaaccctaacagtggtaacacaggctatgcacagaagttccagggcagagtcaccatgaccaggaacacctccataagcacagcctacttggagctgaccagcctgacatctgacgacacggccgtctattactgtgccagaggtgacacaggcgacttctttgaccattggggccagggaaccctggtcaccgtctcctca

SEQ ID NO: 141 - protein, 239 aa, synthetic construct
DIQMTQSPSFLSASVGDRVTITCRASQGISSYLAWYQQKPGKAPKLLIYSASSLQSGVPSRFSGSGSGTDFTLTISSLQPEDFATYYCQQSYNSPTFGQGTRLEIKGGGGSGGGGSGGGGSQVQLVQSGAEVKKPGASVKVSCKASGYTFTGYYMHWVRQAPGQGLEWMGWMNPNSGNTGYAQKFQGRVTMTRNTSISTAYLELTSLTSDDTAVYYCARGDTGDFFDHWGQGTLVTVSS

SEQ ID NO: 142 - protein, 14 aa, synthetic construct
TGTSSDVGASNSVS

SEQ ID NO: 143 - protein, 7 aa, synthetic construct
DVSNRPS

SEQ ID NO: 144 - protein, 11 aa, synthetic construct
SSYTGSSTFVV

SEQ ID NO: 145 - protein, 7 aa, synthetic construct
GFTFSSY

SEQ ID NO: 146 - protein, 6 aa, synthetic construct
RQDGSE

SEQ ID NO: 147 - protein, 13 aa, synthetic construct
AREQDTAMVAFDY

SEQ ID NO: 148 - DNA, 333 bp, synthetic construct
  source 1..333: mol_type = other DNA
cagtctgtgttgactcagcctgcctccgtgtctgggtctcctggacagtcgatcaccatctcctgcactggaaccagcagtgatgttggtgcttctaactctgtctcctggtaccagcgacaccccggcaaaccccccaaactcatgatttatgatgtcagtaatcggccctcagggatttctaatcgcttctctggctccaagtctggcaacacggcctccctgaccatctctgggctccagtctgaggacgaggctgattattactgcagctcatatacaggcagcagcacttttgtggtattcggcggaggaacccagctgatcatttta

SEQ ID NO: 149 - protein, 111 aa, synthetic construct
QSVLTQPASVSGSPGQSITISCTGTSSDVGASNSVSWYQRHPGKPPKLMIYDVSNRPSGISNRFSGSKSGNTASLTISGLQSEDEADYYCSSYTGSSTFVVFGGGTQLIIL

SEQ ID NO: 150 - DNA, 45 bp, synthetic construct; identical to SEQ ID NO: 10
  source 1..45: mol_type = other DNA

SEQ ID NO: 151 - protein, 15 aa, synthetic construct
GGGGSGGGGSGGGGS

SEQ ID NO: 152 - DNA, 360 bp, synthetic construct
  source 1..360: mol_type = other DNA
gaggtgcaactggtggagtctgggggaggcgtggtccagcctggggggtccctgagactctcctgtgcagcctctggattcacctttagtagctattggatgagctgggtccgccaggctccagggaaggggctggagtgggtggccaacataaggcaagatggaagtgagaaatactatgtggactctgtgaagggccgattcaccatctccagagacaacgccaagaactcactgtatctgcaaatgaacagcctgagagccgaggacgcggctgtgtattactgtgcgagagaacaggatacagctatggtcgcctttgactactggggccagggaaccctggtcaccgtctcctca

SEQ ID NO: 153 - protein, 120 aa, synthetic construct
EVQLVESGGGVVQPGGSLRLSCAASGFTFSSYWMSWVRQAPGKGLEWVANIRQDGSEKYYVDSVKGRFTISRDNAKNSLYLQMNSLRAEDAAVYYCAREQDTAMVAFDYWGQGTLVTVSS

SEQ ID NO: 154 - DNA, 738 bp, synthetic construct
  source 1..738: mol_type = other DNA
cagtctgtgttgactcagcctgcctccgtgtctgggtctcctggacagtcgatcaccatctcctgcactggaaccagcagtgatgttggtgcttctaactctgtctcctggtaccagcgacaccccggcaaaccccccaaactcatgatttatgatgtcagtaatcggccctcagggatttctaatcgcttctctggctccaagtctggcaacacggcctccctgaccatctctgggctccagtctgaggacgaggctgattattactgcagctcatatacaggcagcagcacttttgtggtattcggcggaggaacccagctgatcattttaggtggaggcggttccggaggcggtggcagcggcggtggcggttcggaggtgcaactggtggagtctgggggaggcgtggtccagcctggggggtccctgagactctcctgtgcagcctctggattcacctttagtagctattggatgagctgggtccgccaggctccagggaaggggctggagtgggtggccaacataaggcaagatggaagtgagaaatactatgtggactctgtgaagggccgattcaccatctccagagacaacgccaagaactcactgtatctgcaaatgaacagcctgagagccgaggacgcggctgtgtattactgtgcgagagaacaggatacagctatggtcgcctttgactactggggccagggaaccctggtcaccgtctcctca

SEQ ID NO: 155 - protein, 246 aa, synthetic construct
QSVLTQPASVSGSPGQSITISCTGTSSDVGASNSVSWYQRHPGKPPKLMIYDVSNRPSGISNRFSGSKSGNTASLTISGLQSEDEADYYCSSYTGSSTFVVFGGGTQLIILGGGGSGGGGSGGGGSEVQLVESGGGVVQPGGSLRLSCAASGFTFSSYWMSWVRQAPGKGLEWVANIRQDGSEKYYVDSVKGRFTISRDNAKNSLYLQMNSLRAEDAAVYYCAREQDTAMVAFDYWGQGTLVTVSS

SEQ ID NO: 156 - protein, 11 aa, synthetic construct
QASQDISNYLN

SEQ ID NO: 157 - protein, 7 aa, synthetic construct
DASNLET

SEQ ID NO: 158 - protein, 9 aa, synthetic construct
QQANSFPFT

SEQ ID NO: 159 - protein, 7 aa, synthetic construct
GYSFTRN

SEQ ID NO: 160 - protein, 6 aa, synthetic construct
NPNNGD

SEQ ID NO: 161 - protein, 9 aa, synthetic construct
ARGFWSFDV

SEQ ID NO: 162 - DNA, 321 bp, synthetic construct
  source 1..321: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagatttcactctcaccatcagcagcccgcagcctgaagatcttgcaacttactattgtcaacaggccaacagtttcccgttcacttttggccaggggaccaaggtggagatcaaa

SEQ ID NO: 163 - protein, 107 aa, synthetic construct
DIQMTQSPSSLSASVGDRVTITCQASQDISNYLNWYQQKPGKAPKLLIYDASNLETGVPSRFSGSGSGTDFTLTISSPQPEDLATYYCQQANSFPFTFGQGTKVEIK

SEQ ID NO: 164 - DNA, 45 bp, synthetic construct; identical to SEQ ID NO: 10
  source 1..45: mol_type = other DNA

SEQ ID NO: 165 - protein, 15 aa, synthetic construct
GGGGSGGGGSGGGGS

SEQ ID NO: 166 - DNA, 348 bp, synthetic construct
  source 1..348: mol_type = other DNA
caggtgcagctggtgcagtctggggctgaggtgaagaagcctgggtcctcggtgaaggtctcctgcaaggcttctggatacagcttcacgagaaatgatatcacctgggtccgacaggccaatggacgggggcttgagtggctgggacgcttgaacccgaacaatggtgacacagtctatgcagaggtcttccagggcagagtcgccatgactagagacacctccaccgacactgcctacatggagctcgccagtcttaaatctgacgacacggccgtctatttctgtgcgagaggcttttggtcttttgatgtctggggccaagggacaatggtcaccgtctcttca

SEQ ID NO: 167 - protein, 116 aa, synthetic construct
QVQLVQSGAEVKKPGSSVKVSCKASGYSFTRNDITWVRQANGRGLEWLGRLNPNNGDTVYAEVFQGRVAMTRDTSTDTAYMELASLKSDDTAVYFCARGFWSFDVWGQGTMVTVSS

SEQ ID NO: 168 - DNA, 714 bp, synthetic construct
  source 1..714: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagatttcactctcaccatcagcagcccgcagcctgaagatcttgcaacttactattgtcaacaggccaacagtttcccgttcacttttggccaggggaccaaggtggagatcaaaggtggaggcggttccggaggcggtggcagcggcggtggcggttcgcaggtgcagctggtgcagtctggggctgaggtgaagaagcctgggtcctcggtgaaggtctcctgcaaggcttctggatacagcttcacgagaaatgatatcacctgggtccgacaggccaatggacgggggcttgagtggctgggacgcttgaacccgaacaatggtgacacagtctatgcagaggtcttccagggcagagtcgccatgactagagacacctccaccgacactgcctacatggagctcgccagtcttaaatctgacgacacggccgtctatttctgtgcgagaggcttttggtcttttgatgtctggggccaagggacaatggtcaccgtctcttca

SEQ ID NO: 169 - protein, 238 aa, synthetic construct
DIQMTQSPSSLSASVGDRVTITCQASQDISNYLNWYQQKPGKAPKLLIYDASNLETGVPSRFSGSGSGTDFTLTISSPQPEDLATYYCQQANSFPFTFGQGTKVEIKGGGGSGGGGSGGGGSQVQLVQSGAEVKKPGSSVKVSCKASGYSFTRNDITWVRQANGRGLEWLGRLNPNNGDTVYAEVFQGRVAMTRDTSTDTAYMELASLKSDDTAVYFCARGFWSFDVWGQGTMVTVSS

SEQ ID NO: 170 - protein, 7 aa, synthetic construct
SFAFSAY

SEQ ID NO: 171 - protein, 6 aa, synthetic construct
TGSGGT

SEQ ID NO: 172 - protein, 13 aa, synthetic construct
VSHLGGTYGSGFS

SEQ ID NO: 173 - DNA, 363 bp, synthetic construct
  source 1..363: mol_type = other DNA
caggtgcagctggtgcagtctgggggaggcttggtacagcctggagggtccctgagactctcctgtgcagcctcttctttcgctttctctgcttatgaaatgagctgggtccgccaggctccaggacaacggcttgagtgggtctcaggaatcacaggcagtggtggtaccacatattacgaagactcggtgaagggccgattcaccatctccagagacaattccaagaacacgctgtatctgcaaatgaacaccctgagagccgaggacacagccatgtattactgtgtttctcatcttggtgggacttacgggtcgggcttttcctggggccaggggaccctggtcaccgtctcctcaagt

SEQ ID NO: 174 - protein, 121 aa, synthetic construct
QVQLVQSGGGLVQPGGSLRLSCAASSFAFSAYEMSWVRQAPGQRLEWVSGITGSGGTTYYEDSVKGRFTISRDNSKNTLYLQMNTLRAEDTAMYYCVSHLGGTYGSGFSWGQGTLVTVSSS

SEQ ID NO: 175 - protein, 7 aa, synthetic construct
DFAFDYY

SEQ ID NO: 176 - protein, 6 aa, synthetic construct
SGSGGT

SEQ ID NO: 177 - protein, 9 aa, synthetic construct
ATEDPSSVY

SEQ ID NO: 178 - DNA, 348 bp, synthetic construct
  source 1..348: mol_type = other DNA
caggtgcagctggtgcagtctgggggaggcttggtacagcctggagggtccctgagactctcctgtgcagcctctgatttcgctttcgattattatgaaatgagctgggtccgccaggctccaggacaagggcttgagtgggtctcatctattagtggtagtggtggtaccacactctacgcagactctgtgaagggccgattcaccatctccagagacaattccaagaacacgctgtatctgcaaatgaataccctgagagccgaggacacagctttgtattactgtgcaacagaggacccttccagtgtctactggggccagggaacgctggtcaccgtctcctca

SEQ ID NO: 179 - protein, 116 aa, synthetic construct
QVQLVQSGGGLVQPGGSLRLSCAASDFAFDYYEMSWVRQAPGQGLEWVSSISGSGGTTLYADSVKGRFTISRDNSKNTLYLQMNTLRAEDTALYYCATEDPSSVYWGQGTLVTVSS

SEQ ID NO: 180 - protein, 7 aa, synthetic construct
SFSFSDY

SEQ ID NO: 181 - protein, 6 aa, synthetic construct
SGSGGS

SEQ ID NO: 182 - protein, 17 aa, synthetic construct
VRVPLGHCSSTDCANPY

SEQ ID NO: 183 - DNA, 372 bp, synthetic construct
  source 1..372: mol_type = other DNA
caggtgcagctggtgcagtctgggggaggcttggtacagcctggagggtccctgagactctcctgtgcagcctcttctttctctttctctgattatgaaatgagctgggtccgccaggctccaggacaacggcttgagtgggtctcagctattagtggtagtggtggtagcacatactacgcagactctgtgaagggccgattcaccatctccagagacaattccaagaacacgctgtatctgcaaatgaacaccctgagagccgaggacacagccacgtattactgtgtgagagtgcccctaggacattgtagtagtaccgactgcgcaaatccctactggggccagggaaccctggtcaccgtctcctca

SEQ ID NO: 184 - protein, 124 aa, synthetic construct
QVQLVQSGGGLVQPGGSLRLSCAASSFSFSDYEMSWVRQAPGQRLEWVSAISGSGGSTYYADSVKGRFTISRDNSKNTLYLQMNTLRAEDTATYYCVRVPLGHCSSTDCANPYWGQGTLVTVSS

SEQ ID NO: 185 - protein, 7 aa, synthetic construct
SFDFADY

SEQ ID NO: 186 - protein, 6 aa, synthetic construct
SGSGGD

SEQ ID NO: 187 - protein, 11 aa, synthetic construct
AKVGPKWELVD

SEQ ID NO: 188 - DNA, 354 bp, synthetic construct
  source 1..354: mol_type = other DNA
caggtgcagctggtgcagtctgggggaggcttggtacagcctggagggtccctgagactctcctgtgcagcctcttctttcgatttcgctgattatgaaatgagctgggtccgccaggctccaggacaacgccttgagtgggtctccactgtttctggtagcggtggcgacacatggtacgcagactcagtgaagggccgattcaccatctccagagacaattccaagaacacgctgtatctgcaaatgaacaccctgagagccgaggacacggctgtatattactgtgcaaaagtagggcctaagtgggagttagtggactggggccagggaaccctggtcaccgtctcctca

SEQ ID NO: 189 - protein, 118 aa, synthetic construct
QVQLVQSGGGLVQPGGSLRLSCAASSFDFADYEMSWVRQAPGQRLEWVSTVSGSGGDTWYADSVKGRFTISRDNSKNTLYLQMNTLRAEDTAVYYCAKVGPKWELVDWGQGTLVTVSS

SEQ ID NO: 190 - protein, 7 aa, synthetic construct
SFSFSDY

SEQ ID NO: 191 - protein, 6 aa, synthetic construct
SGDGGS

SEQ ID NO: 192 - protein, 17 aa, synthetic construct
ARDPYYYGSGRITKLDY

SEQ ID NO: 193 - DNA, 372 bp, synthetic construct
  source 1..372: mol_type = other DNA
caggtgcagctggtgcagtctgggggaggcttggtacagcctggagggtccctgagactctcctgtgcagcctcttctttctctttctctgattatgaaatgagctgggtccgccaggctccagggaaggggctggagtgggtctctcttattagtggggatggtggtagcacatactatgcagactcagtgaagggccgatttaccatctccagagacaattccaagaacacgctgtatctgcaaatgaacaccctgagagccgaggacacagccatgtattactgtgcgagagacccatattactatggttcggggagaataacaaaacttgactactggggccagggaaccctggtcaccgtctcctca

SEQ ID NO: 194 - protein, 124 aa, synthetic construct
QVQLVQSGGGLVQPGGSLRLSCAASSFSFSDYEMSWVRQAPGKGLEWVSLISGDGGSTYYADSVKGRFTISRDNSKNTLYLQMNTLRAEDTAMYYCARDPYYYGSGRITKLDYWGQGTLVTVSS

SEQ ID NO: 195 - protein, 7 aa, synthetic construct
GFSFSTY

SEQ ID NO: 196 - protein, 6 aa, synthetic construct
IGNSGR

SEQ ID NO: 197 - protein, 12 aa, synthetic construct
ARSRGGFDAFDI

SEQ ID NO: 198 - DNA, 357 bp, synthetic construct
  source 1..357: mol_type = other DNA
caggtgcagctggtgcagtctgggggaggcttggtacagcctggagggtccctgagactctcctgtgcagcctctggattcagcttcagtacttatgaaatgagctgggtccgccaggctccaggacaacggcttgagtggctctcaggcatcattggtaatagtggtcgcacatactactcagactccgtgaagggccgattcaccatctccagagacaattccaagaacacgctgtatctgcaaatgaacaccctgagagccgaggacacagccatgtattactgtgcacgatctaggggggggtttgatgcttttgatatctggggccaagggacaatggtcaccgtctcctca

SEQ ID NO: 199 - protein, 119 aa, synthetic construct
QVQLVQSGGGLVQPGGSLRLSCAASGFSFSTYEMSWVRQAPGQRLEWLSGIIGNSGRTYYSDSVKGRFTISRDNSKNTLYLQMNTLRAEDTAMYYCARSRGGFDAFDIWGQGTMVTVSS

SEQ ID NO: 200 - protein, 7 aa, synthetic construct
DFSFASY

SEQ ID NO: 201 - protein, 5 aa, synthetic construct
DNGGK

SEQ ID NO: 202 - protein, 11 aa, synthetic construct
VRDGGVAGPPT

SEQ ID NO: 203 - DNA, 351 bp, synthetic construct
  source 1..351: mol_type = other DNA
caggtgcagctggtgcagtctgggggaggcttggtacagcctggagggtccctgagactctcctgtgcagcctctgatttctctttcgcttcttatgaaatgagctgggtccgccaggctccaggacaagggcttgagtgggtcgcacttattgataacggtggcaaaacatattacgcagactcggtgaagggccgattcaccatctccagagacaattccaagaacacgctgtatctgcaaatgaacaccctgagagccgaggacgcggctttgtattactgtgtgagagacgggggagtggccggtccccccacctggggccagggaaccctggtcaccgtctcctca

SEQ ID NO: 204 - protein, 117 aa, synthetic construct
QVQLVQSGGGLVQPGGSLRLSCAASDFSFASYEMSWVRQAPGQGLEWVALIDNGGKTYYADSVKGRFTISRDNSKNTLYLQMNTLRAEDAALYYCVRDGGVAGPPTWGQGTLVTVSS

SEQ ID NO: 205 - protein, 7 aa, synthetic construct
DFSFSYY

SEQ ID NO: 206 - protein, 6 aa, synthetic construct
SGSGDS

SEQ ID NO: 207 - protein, 15 aa, synthetic construct
MTDGETWGTHRYTVY

SEQ ID NO: 208 - DNA, 366 bp, synthetic construct
  source 1..366: mol_type = other DNA
caggtgcagctggtgcagtctgggggaggcttggtacagcctggagggtccctgagactctcctgtgcagcctctgatttctctttctcttattatgaaatgagctgggtccgccaggctccagggaagggtctagagtgggtctcaactattagtggtagtggtgatagcacatactacgcagactcggtgaagggccgattcaccatctccagagacaataccaagaacacgctgtatctgcaaatgaacaccctgagagccgaggacacagccacgtattactgtatgacagatggggaaacttgggggactcatcgttataccgtctattggggccagggagccctggtcaccgtctcctca

SEQ ID NO: 209 - protein, 122 aa, synthetic construct
QVQLVQSGGGLVQPGGSLRLSCAASDFSFSYYEMSWVRQAPGKGLEWVSTISGSGDSTYYADSVKGRFTISRDNTKNTLYLQMNTLRAEDTATYYCMTDGETWGTHRYTVYWGQGALVTVSS

SEQ ID NO: 210 - protein, 7 aa, synthetic construct
DFSFYFY

SEQ ID NO: 211 - protein, 6 aa, synthetic construct
SGSGDS

SEQ ID NO: 212 - protein, 13 aa, synthetic construct
ARDPEVGGITGEY

SEQ ID NO: 213 - DNA, 360 bp, synthetic construct
  source 1..360: mol_type = other DNA
caggtgcagctggtgcagtctgggggaggcttggtacagcctggagggtccctgagactctcctgtgcagcctctgatttctctttctatttttatgaaatgagatgggtccgccaggctccaggacaaggccttgagtgggtctcaactattagtggtagtggtgatagcacatactacgcagactcagtgaagggccgattcaccatctccagagacaattccaagaacacgctgtatctgcaaatgaacaccctgagagccgaggacacggccgtatattactgtgcgagagatcccgaagtgggaggtattacaggggagtactggggccagggcaccctggtcaccgtctcctca

SEQ ID NO: 214 - protein, 120 aa, synthetic construct
QVQLVQSGGGLVQPGGSLRLSCAASDFSFYFYEMRWVRQAPGQGLEWVSTISGSGDSTYYADSVKGRFTISRDNSKNTLYLQMNTLRAEDTAVYYCARDPEVGGITGEYWGQGTLVTVSS

SEQ ID NO: 215 - protein, 7 aa, synthetic construct
AFDFASY

SEQ ID NO: 216 - protein, 6 aa, synthetic construct
SGSGGS

SEQ ID NO: 217 - protein, 11 aa, synthetic construct
VKHTIRGVPEV

SEQ ID NO: 218 - DNA, 354 bp, synthetic construct
  source 1..354: mol_type = other DNA
caggtgcagctggtgcagtctgggggaggcttggtacagcctggagggtccctgagactctcctgtgcagcctctgctttcgatttcgcttcttatgaaatgagctgggtccgccaggctccaggacaacgccttgagtgggtctcaggcattagtggtagtggtggttccacatactacgcagactcggtgaagggccgattcaccatctccagagacaattccaagaacacgctgtatctgcaaatgaacaccctgagagccgaggacacggccttatattactgtgtcaaacacacgattcggggagttccggaggtctggggccaagggactatggtcaccgtctcctca

SEQ ID NO: 219 - protein, 118 aa, synthetic construct
QVQLVQSGGGLVQPGGSLRLSCAASAFDFASYEMSWVRQAPGQRLEWVSGISGSGGSTYYADSVKGRFTISRDNSKNTLYLQMNTLRAEDTALYYCVKHTIRGVPEVWGQGTMVTVSS

SEQ ID NO: 220 - protein, 7 aa, synthetic construct
YFDFDDY

SEQ ID NO: 221 - protein, 6 aa, synthetic construct
SGGGRD

SEQ ID NO: 222 - protein, 19 aa, synthetic construct
ARDPYYQVDDKSAYGAFDI

SEQ ID NO: 223 - DNA, 378 bp, synthetic construct
  source 1..378: mol_type = other DNA
caggtgcagctggtgcagtctgggggaggcttggtacagcctggagggtccctgagactctcctgtgcagcctcttatttcgatttcgatgattatgaaatgagctgggtccgccaggctccagggaaggggctagagtgggtggcagttattagtggtggtggtcgtgacacatggtacgcagactccgtgaagggccgattcaccatctccagagacaattccaagaacacgctgtatctgcaaatgaacaccctgagagccgaggacacagctgtgtattactgtgcgagagatccctattaccaagtcgatgataaaagtgcttatggggcttttgatatttggggccaagggacagcggtcaccgtctcctca

SEQ ID NO: 224 - protein, 126 aa, synthetic construct
QVQLVQSGGGLVQPGGSLRLSCAASYFDFDDYEMSWVRQAPGKGLEWVAVISGGGRDTWYADSVKGRFTISRDNSKNTLYLQMNTLRAEDTAVYYCARDPYYQVDDKSAYGAFDIWGQGTAVTVSS

SEQ ID NO: 225 - protein, 7 aa, synthetic construct
AFDFYDY

SEQ ID NO: 226 - protein, 6 aa, synthetic construct
SNSGDA

SEQ ID NO: 227 - protein, 9 aa, synthetic construct
TTIEDTLPS

SEQ ID NO: 228 - DNA, 348 bp, synthetic construct
  source 1..348: mol_type = other DNA
caggtgcagctggtgcagtctgggggaggcttggtacagcctggagggtccctgagactctcctgtgcagcctctgctttcgatttctatgattatgaaatgagctgggtccgccaggctccaggacaagggcttgagtgggtctcggctattagtaatagtggtgatgccacatactacgcagactcagtgaagggccgattcaccatctccagagacaattccaagaacacgctgtatctgcaaatgaacaccctgagagccgaggacacagccgtatattactgtaccacaatagaggatacattaccctcctggggccagggaaccctggtcaccgtctcctca

SEQ ID NO: 229 - protein, 116 aa, synthetic construct
QVQLVQSGGGLVQPGGSLRLSCAASAFDFYDYEMSWVRQAPGQGLEWVSAISNSGDATYYADSVKGRFTISRDNSKNTLYLQMNTLRAEDTAVYYCTTIEDTLPSWGQGTLVTVSS

SEQ ID NO: 230 - protein, 7 aa, synthetic construct
SFDFSSY

SEQ ID NO: 231 - protein, 6 aa, synthetic construct
SGSGDS

SEQ ID NO: 232 - protein, 14 aa, synthetic construct
ARDPYYGGEYYFDY

SEQ ID NO: 233 - DNA, 363 bp, synthetic construct
  source 1..363: mol_type = other DNA
caggtgcagctggtgcagtctgggggaggcttggtacagcctggagggtccctgagactctcctgtgcagcctcttctttcgatttctcttcttatgaaatgagctgggtccgccaggctccaggacaagggcttgagtgggtctctcttattagtggtagtggtgatagcacatactacgcagactccgtgaagggccgattcaccatctccagagacaattccaagaacacgctgtatctgcaaatgaacaccctgagagccgaggacacggctttgtattactgtgcaagagacccctactacggtggcgaatattactttgactactggggccagggaaccctggtcaccgtctcctca

SEQ ID NO: 234 - protein, 121 aa, synthetic construct
QVQLVQSGGGLVQPGGSLRLSCAASSFDFSSYEMSWVRQAPGQGLEWVSLISGSGDSTYYADSVKGRFTISRDNSKNTLYLQMNTLRAEDTALYYCARDPYYGGEYYFDYWGQGTLVTVSS

SEQ ID NO: 235 - protein, 7 aa, synthetic construct
AFSFSSY

SEQ ID NO: 236 - protein, 6 aa, synthetic construct
NGSGGS

SEQ ID NO: 237 - protein, 12 aa, synthetic construct
ASSYSSSSQFEY

SEQ ID NO: 238 - DNA, 357 bp, synthetic construct
  source 1..357: mol_type = other DNA
caggtgcagctggtgcagtctgggggaggcttggtacagcctggagggtccctgagactctcctgtgcagcctctgctttctctttctcttcttatgaaatgagctgggtccgccaggctccaggacaacggcttgagtgggtcgcacatattaatggtagtggtggtagcacatactacgcagactcggtgaagggccgattcaccatctccagagacaattccaagaacacgctgtatctgcaaatgaacaccctgagagccgaggacacagccacgtattactgtgcaagttcgtatagcagctcgtcgcaatttgagtactggggccagggaaccctggtcaccgtctcctca

SEQ ID NO: 239 - protein, 119 aa, synthetic construct
QVQLVQSGGGLVQPGGSLRLSCAASAFSFSSYEMSWVRQAPGQRLEWVAHINGSGGSTYYADSVKGRFTISRDNSKNTLYLQMNTLRAEDTATYYCASSYSSSSQFEYWGQGTLVTVSS

SEQ ID NO: 240 - protein, 7 aa, synthetic construct
DFSFADY

SEQ ID NO: 241 - protein, 6 aa, synthetic construct
SGDGGS

SEQ ID NO: 242 - protein, 14 aa, synthetic construct
ARDPDSGSYWGGDY

SEQ ID NO: 243 - DNA, 363 bp, synthetic construct
  source 1..363: mol_type = other DNA
caggtgcagctggtgcagtctgggggaggcttggtacagcctggagggtccctgagactctcctgtgcagcctctgatttctctttcgctgattatgaaatgagctgggtccgccaggctccaggacaaggccttgagtgggtctctcttattagtggggatggtggtagcacatactatgcagactcggtgaagggccgattcaccatctccagagacaattccaagaacacgctgtatctgcaaatgaacaccctgagagccgaggacacagccgtatattactgtgcgagagatcccgatagtgggagctactgggggggtgactactggggccagggaaccctggtcaccgtctcctca

SEQ ID NO: 244 - protein, 121 aa, synthetic construct
QVQLVQSGGGLVQPGGSLRLSCAASDFSFADYEMSWVRQAPGQGLEWVSLISGDGGSTYYADSVKGRFTISRDNSKNTLYLQMNTLRAEDTAVYYCARDPDSGSYWGGDYWGQGTLVTVSS

SEQ ID NO: 245 - protein, 7 aa, synthetic construct
DFYFADY

SEQ ID NO: 246 - protein, 6 aa, synthetic construct
SGDGGS

SEQ ID NO: 247 - protein, 7 aa, synthetic construct
ARDPWDV

SEQ ID NO: 248 - DNA, 342 bp, synthetic construct
  source 1..342: mol_type = other DNA
caggtgcagctggtgcagtctgggggaggcttggtacagcctggagggtccctgagactctcctgtgcagcctctgatttctatttcgctgattatgaaatgagctgggtccgccaggctccagggaagggtctggagtgggtctctcttattagtggggatggtggtagcacatactatgcagactcggtgaagggccgattcaccatctccagagacaattccaagaacacgctgtatctgcaaatgaacaccctgagagccgaggacacagccacgtattactgtgcaagagatccatgggacgtctggggccaagggaccacggtcaccgtctcctca

SEQ ID NO: 249 - protein, 114 aa, synthetic construct
QVQLVQSGGGLVQPGGSLRLSCAASDFYFADYEMSWVRQAPGKGLEWVSLISGDGGSTYYADSVKGRFTISRDNSKNTLYLQMNTLRAEDTATYYCARDPWDVWGQGTTVTVSS

SEQ ID NO: 250 - protein, 7 aa, synthetic construct
AFDFDSY

SEQ ID NO: 251 - protein, 6 aa, synthetic construct
SGHGGN

SEQ ID NO: 252 - protein, 12 aa, synthetic construct
VRGDEAEMSTIR

SEQ ID NO: 253 - DNA, 357 bp, synthetic construct
  source 1..357: mol_type = other DNA
caggtgcagctggtgcagtctgggggaggcttggtacagcctggagggtccctgagactctcctgtgcagcctctgctttcgatttcgattcttatgaaatgagctgggtccgccaggctccaggacaacggcttgagtgggtctcaactattagtggtcatggtggtaacacatactacgcagactccgtgaagggccgattcaccatctccagagacaattccaagaacacgctgtatctgcaaatgaacaccctgagagccgaggacacggctgtatattactgtgtgagaggggacgaggctgagatgtctacaatacgctggggccagggaaccctggtcaccgtctcctca

SEQ ID NO: 254 - protein, 119 aa, synthetic construct
QVQLVQSGGGLVQPGGSLRLSCAASAFDFDSYEMSWVRQAPGQRLEWVSTISGHGGNTYYADSVKGRFTISRDNSKNTLYLQMNTLRAEDTAVYYCVRGDEAEMSTIRWGQGTLVTVSS

SEQ ID NO: 255 - protein, 7 aa, synthetic construct
DFDFDAY

SEQ ID NO: 256 - protein, 5 aa, synthetic construct
YSDGS

SEQ ID NO: 257 - protein, 14 aa, synthetic construct
AREKDNGDYGDFES

SEQ ID NO: 258 - DNA, 360 bp, synthetic construct
  source 1..360: mol_type = other DNA
caggtgcagctggtgcagtctgggggaggcttggtacagcctggagggtccctgagactctcctgtgcagcctctgatttcgatttcgatgcttatgaaatgagctgggtccgccaggctccaggacaagggcttgagtgggtctcagtgatttatagcgatggtagcacatactacgcagactccgtgaagggccgatttaccatctccagagacaattccaagaacacgctgtatctgcaaatgaacaccctgagagccgaggacacagccatatattactgtgcgagagaaaaggacaacggtgattacggggactttgagtcctggggccagggaaccctggtcaccgtctcctca

SEQ ID NO: 259 - protein, 120 aa, synthetic construct
QVQLVQSGGGLVQPGGSLRLSCAASDFDFDAYEMSWVRQAPGQGLEWVSVIYSDGSTYYADSVKGRFTISRDNSKNTLYLQMNTLRAEDTAIYYCAREKDNGDYGDFESWGQGTLVTVSS

SEQ ID NO: 260 - protein, 7 aa, synthetic construct
SFAFSDY

SEQ ID NO: 261 - protein, 6 aa, synthetic construct
SGSGGS

SEQ ID NO: 262 - protein, 15 aa, synthetic construct
ARDPADYDILTGGDY

SEQ ID NO: 263 - DNA, 366 bp, synthetic construct
  source 1..366: mol_type = other DNA
caggtgcagctggtgcagtctgggggaggcttggtacagcctggagggtccctgagactctcctgtgcagcctcttctttcgctttctctgattatgaaatgagctgggtccgccaggctccaggacaagggcttgagtgggtctcaactataagtggtagtggtggtagcacatactacgcagactcagtgaagggccgattcaccatctccagagacaattccaagaacacgctgtatctgcaaatgaacaccctgagagccgaggacacggctgtatattactgtgcgagagatccagccgattacgatattttgactggtggggactactggggccagggaactctggtcaccgtctcctca

SEQ ID NO: 264 - protein, 122 aa, synthetic construct
QVQLVQSGGGLVQPGGSLRLSCAASSFAFSDYEMSWVRQAPGQGLEWVSTISGSGGSTYYADSVKGRFTISRDNSKNTLYLQMNTLRAEDTAVYYCARDPADYDILTGGDYWGQGTLVTVSS

SEQ ID NO: 265 - protein, 7 aa, synthetic construct
DFDFSDY

SEQ ID NO: 266 - protein, 6 aa, synthetic construct
SDSGDT

SEQ ID NO: 267 - protein, 17 aa, synthetic construct
AKAPYYDILTGDDAFDT

SEQ ID NO: 268 - DNA, 372 bp, synthetic construct
  source 1..372: mol_type = other DNA
caggtgcagctggtgcagtctgggggaggcttggtacagcctggagggtccctgagactctcctgtgcagcctctgatttcgatttctctgattatgaaatgagctgggtccgccaggctccagggaaggggctggagtgggtctcagctatcagtgatagtggtgataccacatactacgcagactcggtgaagggccgattcaccatctccagagacaattccaagaacacgctgtatctgcaaatgaacaccctgagagccgaggacacagccttgtattactgtgcgaaagccccgtattacgatattttgactggtgatgatgcttttgatacctggggccaagggacaatggtcaccgtctcctca

SEQ ID NO: 269 - protein, 124 aa, synthetic construct
QVQLVQSGGGLVQPGGSLRLSCAASDFDFSDYEMSWVRQAPGKGLEWVSAISDSGDTTYYADSVKGRFTISRDNSKNTLYLQMNTLRAEDTALYYCAKAPYYDILTGDDAFDTWGQGTMVTVSS

SEQ ID NO: 270 - skipped

SEQ ID NO: 271 - protein, 7 aa, synthetic construct
YFDFDAY

SEQ ID NO: 272 - protein, 6 aa, synthetic construct
SGDGGS

SEQ ID NO: 273 - protein, 10 aa, synthetic construct
VRDSGYSSFH

SEQ ID NO: 274 - DNA, 354 bp, synthetic construct
  source 1..354: mol_type = other DNA
caggtgcagctggtgcagtctgggggaggcttggtacagcctggagggtccctgagactctcctgtgcagcctcttatttcgatttcgatgcttatgaaatgagctgggtccgccaggctccagggaagggtctggagtgggtctctcttattagtggggatggtggtagcacatactatgcagactctgtgaagggccgattcaccatctccagagacaattccaagaacacgctgtatctgcaaatgaataccctgagagccgaggacacggccgtttatttttgtgtgagagatagcggctacagctctttccactggggccagggaacccaggtcaccgtctcctcaagt

SEQ ID NO: 275 - protein, 118 aa, synthetic construct
QVQLVQSGGGLVQPGGSLRLSCAASYFDFDAYEMSWVRQAPGKGLEWVSLISGDGGSTYYADSVKGRFTISRDNSKNTLYLQMNTLRAEDTAVYFCVRDSGYSSFHWGQGTQVTVSSS

SEQ ID NO: 276 - protein, 7 aa, synthetic construct
YFDFAYY

SEQ ID NO: 277 - protein, 6 aa, synthetic construct
SGDGGS

SEQ ID NO: 278 - protein, 14 aa, synthetic construct
ARDPYYGGEYYFDY

SEQ ID NO: 279 - DNA, 363 bp, synthetic construct
  source 1..363: mol_type = other DNA
caggtgcagctggtgcagtctgggggaggcttggtacagcctggagggtccctgagactctcctgtgcagcctcttatttcgatttcgcttattatgaaatgagctgggtccgccaggctccagggaagggtctggagtgggtctctcttattagtggggatggtggtagcacatactatgcagactctgtgaagggccgattcaccatctccagagacaattccaagaacacgctgtatctgcaaatgaacaccctgagagccgaggacacggccgtatattactgtgcaagagacccctactacggtggcgaatattactttgactactggggccagggaaccctggtcaccgtctcctca

SEQ ID NO: 280 - protein, 121 aa, synthetic construct
QVQLVQSGGGLVQPGGSLRLSCAASYFDFAYYEMSWVRQAPGKGLEWVSLISGDGGSTYYADSVKGRFTISRDNSKNTLYLQMNTLRAEDTAVYYCARDPYYGGEYYFDYWGQGTLVTVSS

SEQ ID NO: 281 - protein, 7 aa, synthetic construct
AFAFSSY

SEQ ID NO: 282 - protein, 6 aa, synthetic construct
SGDGGS

SEQ ID NO: 283 - protein, 11 aa, synthetic construct
VRETESQDVVY

SEQ ID NO: 284 - DNA, 354 bp, synthetic construct
  source 1..354: mol_type = other DNA
caggtgcagctggtgcagtctgggggaggcttggtacagcctggagggtccctgagactctcctgtgcagcctctgctttcgctttctcttcttatgaaatgagctgggtccgccaggctccagggaaggggctggagtgggtctctcttattagtggggatggtggtagcacatactatgcagactctgtgaagggccgattcaccatctccagagacaataccaagaacacgctgtatctgcaaatgaacaccctgagagccgaggacacagctgtatattactgtgtgagagagactgaatcacaggacgtcgtctactggggccagggaaccctggtcaccgtctcctca

SEQ ID NO: 285 - protein, 118 aa, synthetic construct
QVQLVQSGGGLVQPGGSLRLSCAASAFAFSSYEMSWVRQAPGKGLEWVSLISGDGGSTYYADSVKGRFTISRDNTKNTLYLQMNTLRAEDTAVYYCVRETESQDVVYWGQGTLVTVSS

SEQ ID NO: 286 - protein, 7 aa, synthetic construct
DFDFSDY

SEQ ID NO: 287 - protein, 5 aa, synthetic construct
YSDGG

SEQ ID NO: 288 - protein, 17 aa, synthetic construct
ARDPYYYGSGNLRFNDY

SEQ ID NO: 289 - DNA, 369 bp, synthetic construct
  source 1..369: mol_type = other DNA
caggtgcagctggtgcagtctgggggaggcttggtacagcctggagggtccctgagactctcctgtgcagcctctgatttcgatttctctgattatgaaatgagctgggtccgccaggctccagggaaggggctagagtgggtctcagggatttatagcgatggtggcacatactactcagactcggtgaagggccgattctccatctccagagacaattccaagaacaccctgtatctgcaaatgaacaccctgagagccgaggacacggccgtatattactgtgcgagagatccgtattactatggttcggggaacctaagattcaatgactactggggccagggaaccctggtcaccgtctcctca

SEQ ID NO: 290 - protein, 123 aa, synthetic construct
QVQLVQSGGGLVQPGGSLRLSCAASDFDFSDYEMSWVRQAPGKGLEWVSGIYSDGGTYYSDSVKGRFSISRDNSKNTLYLQMNTLRAEDTAVYYCARDPYYYGSGNLRFNDYWGQGTLVTVSS

SEQ ID NO: 291 - protein, 7 aa, synthetic construct
SFDFDDY

SEQ ID NO: 292 - protein, 6 aa, synthetic construct
SGDGGS

SEQ ID NO: 293 - protein, 13 aa, synthetic construct
ARDPFANQQGGDY

SEQ ID NO: 294 - DNA, 360 bp, synthetic construct
  source 1..360: mol_type = other DNA
caggtgcagctggtgcagtctgggggaggcttggtacagcctggagggtccctgagactctcctgtgcagcctcttctttcgatttcgatgattatgaaatgagctgggtccgccaggctccagggaagggactagagtgggtctctcttattagtggggatggtggtagcacatactatgcagactccgtgaagggccgattcaccatctccagagacaattccaagaacacgctgtatctgcaaatgaacaccctgagagccgaggacacagccgtgtattactgtgcgagagatccatttgccaaccagcagggcggggactactggggccagggaaccctggtcaccgtctcctca

SEQ ID NO: 295 - protein, 120 aa, synthetic construct
QVQLVQSGGGLVQPGGSLRLSCAASSFDFDDYEMSWVRQAPGKGLEWVSLISGDGGSTYYADSVKGRFTISRDNSKNTLYLQMNTLRAEDTAVYYCARDPFANQQGGDYWGQGTLVTVSS

SEQ ID NO: 296 - protein, 11 aa, synthetic construct
RASQSISSWLA

SEQ ID NO: 297 - protein, 7 aa, synthetic construct
TASSLQS

SEQ ID NO: 298 - protein, 9 aa, synthetic construct
QQSYSTPLT

SEQ ID NO: 299 - protein, 7 aa, synthetic construct
GYTFTGY

SEQ ID NO: 300 - protein, 6 aa, synthetic construct
NPNSGG

SEQ ID NO: 301 - protein, 11 aa, synthetic construct
ARGDTGDFFDH

SEQ ID NO: 302 - DNA, 321 bp, synthetic construct
  source 1..321: mol_type = other DNA
gacatccagatgacccagtccccttccaccctgtctgcatctgtaggagacagagtcaccatcacttgccgggctagtcagagtattagtagctggttggcctggtatcagcagaaaccagggaaagcccctaagctcctgatctatactgcatccagtttgcaaagtggggtcccatcaaggttcagtggcagtgaatctgggacagatttcactctcaccatcagcagtctgcaacctgaagattttgcaacttactactgtcaacagagttacagtacccctctcactttcggcggagggaccaaagtggatatcaaa

SEQ ID NO: 303 - protein, 107 aa, synthetic construct
DIQMTQSPSTLSASVGDRVTITCRASQSISSWLAWYQQKPGKAPKLLIYTASSLQSGVPSRFSGSESGTDFTLTISSLQPEDFATYYCQQSYSTPLTFGGGTKVDIK

SEQ ID NO: 304 - DNA, 45 bp, synthetic construct; identical to SEQ ID NO: 10
  source 1..45: mol_type = other DNA

SEQ ID NO: 305 - protein, 15 aa, synthetic construct
GGGGSGGGGSGGGGS

SEQ ID NO: 306 - DNA, 354 bp, synthetic construct
  source 1..354: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagttccagggcagagtcaccctgaccagggatacctccatcaccacagcctacttggagctgaccagcctgacatctgacgacacggccgtctattactgtgccagaggtgacacaggcgacttctttgaccattggggccagggaaccctggtcaccgtctcctca

SEQ ID NO: 307 - protein, 118 aa, synthetic construct
QVQLVQSGAEVKKPGASVKVSCKASGYTFTGYYMHWVRQAPGQGLEWMGWINPNSGGTNYAQKFQGRVTLTRDTSITTAYLELTSLTSDDTAVYYCARGDTGDFFDHWGQGTLVTVSS

SEQ ID NO: 308 - DNA, 720 bp, synthetic construct
  source 1..720: mol_type = other DNA
gacatccagatgacccagtccccttccaccctgtctgcatctgtaggagacagagtcaccatcacttgccgggctagtcagagtattagtagctggttggcctggtatcagcagaaaccagggaaagcccctaagctcctgatctatactgcatccagtttgcaaagtggggtcccatcaaggttcagtggcagtgaatctgggacagatttcactctcaccatcagcagtctgcaacctgaagattttgcaacttactactgtcaacagagttacagtacccctctcactttcggcggagggaccaaagtggatatcaaaggtggaggcggttccggaggcggtggcagcggcggtggcggttcgcaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagttccagggcagagtcaccctgaccagggatacctccatcaccacagcctacttggagctgaccagcctgacatctgacgacacggccgtctattactgtgccagaggtgacacaggcgacttctttgaccattggggccagggaaccctggtcaccgtctcctca

SEQ ID NO: 309 - protein, 240 aa, synthetic construct
DIQMTQSPSTLSASVGDRVTITCRASQSISSWLAWYQQKPGKAPKLLIYTASSLQSGVPSRFSGSESGTDFTLTISSLQPEDFATYYCQQSYSTPLTFGGGTKVDIKGGGGSGGGGSGGGGSQVQLVQSGAEVKKPGASVKVSCKASGYTFTGYYMHWVRQAPGQGLEWMGWINPNSGGTNYAQKFQGRVTLTRDTSITTAYLELTSLTSDDTAVYYCARGDTGDFFDHWGQGTLVTVSS

SEQ ID NO: 310 - protein, 11 aa, synthetic construct
RASQSISSYLN

SEQ ID NO: 311 - protein, 7 aa, synthetic construct
AASSLQS

SEQ ID NO: 312 - protein, 9 aa, synthetic construct
QQSYSTPIT

SEQ ID NO: 313 - protein, 7 aa, synthetic construct
GYTFTGY

SEQ ID NO: 314 - protein, 6 aa, synthetic construct
NPSSGN

SEQ ID NO: 315 - protein, 11 aa, synthetic construct
ARGDTGDSFDY

SEQ ID NO: 316 - DNA, 321 bp, synthetic construct
  source 1..321: mol_type = other DNA
gacatccagatgacccagtctccatcctccctgtctgcatctgtaggagacagagtcaccaccacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgatcaccttcggccaagggacacgactggagattaaa

SEQ ID NO: 317 - protein, 107 aa, synthetic construct
DIQMTQSPSSLSASVGDRVTTTCRASQSISSYLNWYQQKPGKAPKLLIYAASSLQSGVPSRFSGSGSGTDFTLTISSLQPEDFATYYCQQSYSTPITFGQGTRLEIK

SEQ ID NO: 318 - DNA, 45 bp, synthetic construct; identical to SEQ ID NO: 10
  source 1..45: mol_type = other DNA

SEQ ID NO: 319 - protein, 15 aa, synthetic construct
GGGGSGGGGSGGGGS

SEQ ID NO: 320 - DNA, 354 bp, synthetic construct
  source 1..354: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatgaacccgagcagtggaaataccggctatgcacatgaattccagggcagagtcaccatgaccaggaacacttccacaggcacggcctacatggagctgagcggcctgagatctgaggacacggccgtgtattactgtgctagaggtgatactggggactcctttgactactggggccagggaaccctggtcaccgtctcctca

SEQ ID NO: 321 - protein, 118 aa, synthetic construct
QVQLVQSGAEVKKPGASVKVSCKASGYTFTGYYMHWVRQAPGQGLEWMGWMNPSSGNTGYAHEFQGRVTMTRNTSTGTAYMELSGLRSEDTAVYYCARGDTGDSFDYWGQGTLVTVSS

SEQ ID NO: 322 - DNA, 720 bp, synthetic construct
  source 1..720: mol_type = other DNA
gacatccagatgacccagtctccatcctccctgtctgcatctgtaggagacagagtcaccaccacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgatcaccttcggccaagggacacgactggagattaaaggtggaggcggttccggaggcggtggcagcggcggtggcggttcgcaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatgaacccgagcagtggaaataccggctatgcacatgaattccagggcagagtcaccatgaccaggaacacttccacaggcacggcctacatggagctgagcggcctgagatctgaggacacggccgtgtattactgtgctagaggtgatactggggactcctttgactactggggccagggaaccctggtcaccgtctcctca

SEQ ID NO: 323 - protein, 240 aa, synthetic construct
DIQMTQSPSSLSASVGDRVTTTCRASQSISSYLNWYQQKPGKAPKLLIYAASSLQSGVPSRFSGSGSGTDFTLTISSLQPEDFATYYCQQSYSTPITFGQGTRLEIKGGGGSGGGGSGGGGSQVQLVQSGAEVKKPGASVKVSCKASGYTFTGYYMHWVRQAPGQGLEWMGWMNPSSGNTGYAHEFQGRVTMTRNTSTGTAYMELSGLRSEDTAVYYCARGDTGDSFDYWGQGTLVTVSS

SEQ ID NO: 324 - protein, 13 aa, synthetic construct
SGSSSNIGNNYVS

SEQ ID NO: 325 - protein, 7 aa, synthetic construct
DNNKRPS

SEQ ID NO: 326 - protein, 11 aa, synthetic construct
GTWDSSLSAWV

SEQ ID NO: 327 - protein, 7 aa, synthetic construct
GDSVSSN

SEQ ID NO: 328 - protein, 7 aa, synthetic construct
HYRSKWY

SEQ ID NO: 329 - protein, 14 aa, synthetic construct
ARDRYTVRPDAFDI

SEQ ID NO: 330 - DNA, 330 bp, synthetic construct
  source 1..330: mol_type = other DNA
cagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccgagtctggcacgtcagccaccctgggcatcaccggactccagactggggacgaggccgattattactgcggaacatgggatagcagcctgagtgcttgggtgttcggcggaggaacccagctgatcatttta

SEQ ID NO: 331 - protein, 110 aa, synthetic construct
QSVLTQPPSVSAAPGQKVTISCSGSSSNIGNNYVSWYQQLPGTAPKLLIYDNNKRPSGIPDRFSGSESGTSATLGITGLQTGDEADYYCGTWDSSLSAWVFGGGTQLIIL

SEQ ID NO: 332 - DNA, 45 bp, synthetic construct; identical to SEQ ID NO: 10
  source 1..45: mol_type = other DNA

SEQ ID NO: 333 - protein, 15 aa, synthetic construct
GGGGSGGGGSGGGGS

SEQ ID NO: 334 - DNA, 372 bp, synthetic construct
  source 1..372: mol_type = other DNA
caggtacagctgcagcagtcaggtccaggactggtgaagccctcgcagaccctctcactcacctgtgccatctccggagacagtgtctctagcaacagtgctgcttggaactggatcaggcagtccccatcgagaggccttgagtggctgggacggacacactacaggtccaagtggtataatgattatgcagtatctgtgaaaagtcgaataaccatcagcgcagacacttccaagaaccaattctccctgcagctgaactctgtgactcccgaggacacggctgtgtattactgtgcaagagatcgttatacggtccgccctgatgcttttgatatctggggccaagggacaacggtcaccgtctcctca

SEQ ID NO: 335 - protein, 124 aa, synthetic construct
QVQLQQSGPGLVKPSQTLSLTCAISGDSVSSNSAAWNWIRQSPSRGLEWLGRTHYRSKWYNDYAVSVKSRITISADTSKNQFSLQLNSVTPEDTAVYYCARDRYTVRPDAFDIWGQGTTVTVSS

SEQ ID NO: 336 - DNA, 747 bp, synthetic construct
  source 1..747: mol_type = other DNA
cagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccgagtctggcacgtcagccaccctgggcatcaccggactccagactggggacgaggccgattattactgcggaacatgggatagcagcctgagtgcttgggtgttcggcggaggaacccagctgatcattttaggtggaggcggttccggaggcggtggcagcggcggtggcggttcgcaggtacagctgcagcagtcaggtccaggactggtgaagccctcgcagaccctctcactcacctgtgccatctccggagacagtgtctctagcaacagtgctgcttggaactggatcaggcagtccccatcgagaggccttgagtggctgggacggacacactacaggtccaagtggtataatgattatgcagtatctgtgaaaagtcgaataaccatcagcgcagacacttccaagaaccaattctccctgcagctgaactctgtgactcccgaggacacggctgtgtattactgtgcaagagatcgttatacggtccgccctgatgcttttgatatctggggccaagggacaacggtcaccgtctcctca

SEQ ID NO: 337 - protein, 249 aa, synthetic construct
QSVLTQPPSVSAAPGQKVTISCSGSSSNIGNNYVSWYQQLPGTAPKLLIYDNNKRPSGIPDRFSGSESGTSATLGITGLQTGDEADYYCGTWDSSLSAWVFGGGTQLIILGGGGSGGGGSGGGGSQVQLQQSGPGLVKPSQTLSLTCAISGDSVSSNSAAWNWIRQSPSRGLEWLGRTHYRSKWYNDYAVSVKSRITISADTSKNQFSLQLNSVTPEDTAVYYCARDRYTVRPDAFDIWGQGTTVTVSS

SEQ ID NO: 338 - protein, 14 aa, synthetic construct
TGTSRDVGGYNFVS

SEQ ID NO: 339 - protein, 7 aa, synthetic construct
DVTTRPS

SEQ ID NO: 340 - protein, 10 aa, synthetic construct
GSYTASDTML

SEQ ID NO: 341 - protein, 7 aa, synthetic construct
GFTFDDY

SEQ ID NO: 342 - protein, 6 aa, synthetic construct
SWNSGS

SEQ ID NO: 343 - protein, 11 aa, synthetic construct
AREGRPTAFDY

SEQ ID NO: 344 - DNA, 330 bp, synthetic construct
  source 1..330: mol_type = other DNA
cagtctgtgctgactcagcctgcctccgtgtctgggtctcttggacagtcgatcaccatctcctgcactggaaccagccgtgacgttggtggttataactttgtctcctggtaccaacaacacccaggcaaagcccccaaactcataatttttgatgtcactactcggccctcagggctttctgatcgcttctctgcttccaagtccggcaacacggcctccctgaccatctctggcctccaggcggaggacgaggctgattatttctgtggctcatacacagccagtgacactatgttattcggcggagggaccaagctgaccgtccta

SEQ ID NO: 345 - protein, 110 aa, synthetic construct
QSVLTQPASVSGSLGQSITISCTGTSRDVGGYNFVSWYQQHPGKAPKLIIFDVTTRPSGLSDRFSASKSGNTASLTISGLQAEDEADYFCGSYTASDTMLFGGGTKLTVL

SEQ ID NO: 346 - DNA, 45 bp, synthetic construct; identical to SEQ ID NO: 10
  source 1..45: mol_type = other DNA

SEQ ID NO: 347 - protein, 15 aa, synthetic construct
GGGGSGGGGSGGGGS

SEQ ID NO: 348 - DNA, 354 bp, synthetic construct
  source 1..354: mol_type = other DNA
caggtgcagctgttggagtctgggggaggcgtggtccagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccgtctccagagacaattccaagaacacgctgtatcagcaaatgaacggcctgagagctgaagacacggctgtatattactgtgcgagagaggggcggccaacggcctttgactactggggccagggaaccacggtcaccgtctcctca

SEQ ID NO: 349 - protein, 118 aa, synthetic construct
QVQLLESGGGVVQPGRSLRLSCAASGFTFDDYAMHWVRQAPGKGLEWVSGISWNSGSIGYADSVKGRFTVSRDNSKNTLYQQMNGLRAEDTAVYYCAREGRPTAFDYWGQGTTVTVSS

SEQ ID NO: 350 - DNA, 729 bp, synthetic construct
  source 1..729: mol_type = other DNA
cagtctgtgctgactcagcctgcctccgtgtctgggtctcttggacagtcgatcaccatctcctgcactggaaccagccgtgacgttggtggttataactttgtctcctggtaccaacaacacccaggcaaagcccccaaactcataatttttgatgtcactactcggccctcagggctttctgatcgcttctctgcttccaagtccggcaacacggcctccctgaccatctctggcctccaggcggaggacgaggctgattatttctgtggctcatacacagccagtgacactatgttattcggcggagggaccaagctgaccgtcctaggtggaggcggttccggaggcggtggcagcggcggtggcggttcgcaggtgcagctgttggagtctgggggaggcgtggtccagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccgtctccagagacaattccaagaacacgctgtatcagcaaatgaacggcctgagagctgaagacacggctgtatattactgtgcgagagaggggcggccaacggcctttgactactggggccagggaaccacggtcaccgtctcctca

SEQ ID NO: 351 - protein, 243 aa, synthetic construct
QSVLTQPASVSGSLGQSITISCTGTSRDVGGYNFVSWYQQHPGKAPKLIIFDVTTRPSGLSDRFSASKSGNTASLTISGLQAEDEADYFCGSYTASDTMLFGGGTKLTVLGGGGSGGGGSGGGGSQVQLLESGGGVVQPGRSLRLSCAASGFTFDDYAMHWVRQAPGKGLEWVSGISWNSGSIGYADSVKGRFTVSRDNSKNTLYQQMNGLRAEDTAVYYCAREGRPTAFDYWGQGTTVTVSS

SEQ ID NO: 352 - protein, 11 aa, synthetic construct
RASQSVSSYLA

SEQ ID NO: 353 - protein, 7 aa, synthetic construct
DASNRAT

SEQ ID NO: 354 - protein, 9 aa, synthetic construct
QQYGSSPVT

SEQ ID NO: 355 - protein, 7 aa, synthetic construct
GGTFSSY

SEQ ID NO: 356 - protein, 6 aa, synthetic construct
IPIFGT

SEQ ID NO: 357 - protein, 12 aa, synthetic construct
ARDADEFGAFDI

SEQ ID NO: 358 - DNA, 321 bp, synthetic construct
  source 1..321: mol_type = other DNA
gaaattgtgttgacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagacaggttcagtggcagtgggtctgggacagacttcactctcaccatcagcagactggagcctgaagattttgcagtgtattactgtcagcagtatggtagctcacccgtcacttttggccaggggaccaagctggagatcaaa

SEQ ID NO: 359 - protein, 107 aa, synthetic construct
EIVLTQSPATLSLSPGERATLSCRASQSVSSYLAWYQQKPGQAPRLLIYDASNRATGIPDRFSGSGSGTDFTLTISRLEPEDFAVYYCQQYGSSPVTFGQGTKLEIK

SEQ ID NO: 360 - DNA, 45 bp, synthetic construct; identical to SEQ ID NO: 10
  source 1..45: mol_type = other DNA

SEQ ID NO: 361 - protein, 15 aa, synthetic construct
GGGGSGGGGSGGGGS

SEQ ID NO: 362 - DNA, 357 bp, synthetic construct
  source 1..357: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcatcatgaccacagacacatccacgagcacagcccacatggaactgaggagcctgagatctgacgacacggccgtatattactgtgcgagagacgctgatgaattcggagcttttgatatctggggccaagggacaacggtcaccgtctcctcg

SEQ ID NO: 363 - protein, 119 aa, synthetic construct
QVQLVQSGAEVKKPGSSVKVSCKASGGTFSSYAISWVRQAPGQGLEWMGGIIPIFGTANYAQKFQGRVIMTTDTSTSTAHMELRSLRSDDTAVYYCARDADEFGAFDIWGQGTTVTVSS

SEQ ID NO: 364 - DNA, 723 bp, synthetic construct
  source 1..723: mol_type = other DNA
gaaattgtgttgacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagacaggttcagtggcagtgggtctgggacagacttcactctcaccatcagcagactggagcctgaagattttgcagtgtattactgtcagcagtatggtagctcacccgtcacttttggccaggggaccaagctggagatcaaaggtggaggcggttccggaggcggtggcagcggcggtggcggttcgcaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcatcatgaccacagacacatccacgagcacagcccacatggaactgaggagcctgagatctgacgacacggccgtatattactgtgcgagagacgctgatgaattcggagcttttgatatctggggccaagggacaacggtcaccgtctcctcg

SEQ ID NO: 365 - protein, 241 aa, synthetic construct
EIVLTQSPATLSLSPGERATLSCRASQSVSSYLAWYQQKPGQAPRLLIYDASNRATGIPDRFSGSGSGTDFTLTISRLEPEDFAVYYCQQYGSSPVTFGQGTKLEIKGGGGSGGGGSGGGGSQVQLVQSGAEVKKPGSSVKVSCKASGGTFSSYAISWVRQAPGQGLEWMGGIIPIFGTANYAQKFQGRVIMTTDTSTSTAHMELRSLRSDDTAVYYCARDADEFGAFDIWGQGTTVTVSS

SEQ ID NO: 366 - protein, 5 aa, synthetic construct
GGGGS

SEQ ID NO: 367 - protein, 10 aa, synthetic construct
GGGGSGGGGS

SEQ ID NO: 368 - protein, 15 aa, synthetic construct
GGGGSGGGGSGGGGS

SEQ ID NO: 369 - protein, 45 aa, synthetic construct
TTTPAPRPPTPAPTIASQPLSLRPEACRPAAGGAVHTRGLDFACD

SEQ ID NO: 370 - protein, 42 aa, synthetic construct
AAAIEVMYPPPYLDNEKSNGTIIHVKGKHLCPSPLFPGPSKP

SEQ ID NO: 371 - protein, 24 aa, synthetic construct
IYIWAPLAGTCGVLLLSLVITLYC

SEQ ID NO: 372 - protein, 68 aa, synthetic construct
FWVLVVVGGVLACYSLLVTVAFIIFWVRSKRSRLLHSDYMNMTPRRPGPTRKHYQPYAPPRDFAAYRS

SEQ ID NO: 373 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 374 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 375 - protein, 42 aa, synthetic construct
KRGRKKLLYIFKQPFMRPVQTTQEEDGCSCRFPEEEEGGCEL

SEQ ID NO: 376 - protein, 471 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHPVFGGGTQLIILGGGGSGGGGSGGGGSQVQLVQSGAEVKKPGSSVKVSCKASGGTFSSYAISWVRQAPGQGLEWMGRIIPILGIANYAQKFQGRVTITADESTSTAYMELSSLRSEDTAVYYCAREKDDILTGAYYYGMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 377 - protein, 464 aa, synthetic construct
QSVLTQPLSVSVALGQTARITCGGNNIGSKNVHWYQQKAGQAPVLVIQRDSNRPSGIPERFSGSNSGNTATLTISRAQAGDEADYFCQVWDSNTYVFGTGTKLTVLGGGGSGGGGSGGGGSEVQLVESGGGLVKPGGSLRLSCAASGFTFSDYYMSWIRQAPGKGLEWVSYISSSGSTIYYADSVKGRFTISRDNAKNSLYLQMNSLRDEDTAVYYCARQSAEIVGAFDHWGQGTMVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 378 - protein, 463 aa, synthetic construct
QSALTQPPSASGSPGQSVTISCTGTSSDVGGYNYVSWYQQHPGKAPKLMIYEVGKRPSGVPDRFSGSKSGNTASLTVSGLQAEDEADYYCSSYTSSSTRVFGGGTKVTVLGGGGSGGGGSGGGGSEVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDPHMDVWGQGTMVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 379 - protein, 470 aa, synthetic construct
QSVVTQPPSVSAAPGQKVTISCSGSSPNIGKNYVSWYQQLPGTAPKLLIYDNNKRPSGIPDRFSGSKSGTSATLGITGLQTGDEADYYCGTWDSSLSGYVFGTGTKVTVLGGGGSGGGGSGGGGSQVQLVESGGGVVQPGRSLRLSCAASGFTFSSYGMHWVRQAPGKGLEWVAFISHDGSNKYYADSVKGRSTISRDNSKSTLFLQMNSLRDEDTAVYFCAREGQLTTSWDFFDYWGHGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 380 - protein, 467 aa, synthetic construct
DIQLTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKVEIKGGGGSGGGGSGGGGSQVQLVESGGGVVQPGRSLRLSCAASGFTFSSYGMHWVRQAPGKGLEWVAFISHDGSNKYYADSVKGRSTISRDNSKSTLFLQMNSLRDEDTAVYFCAREGQLTTSWDFFDYWGHGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 381 - protein, 471 aa, synthetic construct
QSVLTQPPSVSAAPGQKVSIFCSGGSSNIGKNYVSWYQQLPGTAPKLLIYENQHRPSGIPDRFSGSKSGTSATLGITGLQTGDEADYYCATWDDSLSAGVFGGGTELTVLGGGGSGGGGSGGGGSQLVQSGAEVRKPGESLKISCKGSGYSFTSYWIGWVRQMPGKGLEWMGFIYPGDSDTRYSPSFQGQVTISADKSISTAYLQRSSLKASDTAMYYCARLGGYSGYDSEVFYLDYWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 382 - protein, 463 aa, synthetic construct; identical to SEQ ID NO: 378

SEQ ID NO: 383 - protein, 463 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSGGGGSGGGGSQVQLVQSGAEVKKPGSSVKVSCKASGGTFTSYGISWVRQAPGQGLEWMGWISAYNGNTNYAQKLQGRVTMTTDTSTSTAYMELRSLRAEDTAVYYCALPTADDAFDIWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 384 - protein, 461 aa, synthetic construct
SYELTQPPSVSVAPGKTARITCGGNNIGSKSVHWYQQKPGQAPVLVVYDDSDRPSGIPERFSGSNSGNTATLTISRVEAGDEADYYCQVWDSGSDHPVVSGGGTQLIILGGGGSGGGGSGGGGSQVQLVESGGGLVQPGGSLRLSCAASGFTFSSYAMSWVRQAPGKGLEWVSAIGGSGGSTYYADSVKGRFTISRDNSKNTLYLQMNSLRAEDTAVYYCANIDGTFWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 385 - protein, 462 aa, synthetic construct
DIQMTQSPSFLSASVGDRVTITCRASQGISSYLAWYQQKPGKAPKLLIYSASSLQSGVPSRFSGSGSGTDFTLTISSLQPEDFATYYCQQSYNSPTFGQGTRLEIKGGGGSGGGGSGGGGSQVQLVQSGAEVKKPGASVKVSCKASGYTFTGYYMHWVRQAPGQGLEWMGWMNPNSGNTGYAQKFQGRVTMTRNTSISTAYLELTSLTSDDTAVYYCARGDTGDFFDHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 386 - protein, 469 aa, synthetic construct
QSVLTQPASVSGSPGQSITISCTGTSSDVGASNSVSWYQRHPGKPPKLMIYDVSNRPSGISNRFSGSKSGNTASLTISGLQSEDEADYYCSSYTGSSTFVVFGGGTQLIILGGGGSGGGGSGGGGSEVQLVESGGGVVQPGGSLRLSCAASGFTFSSYWMSWVRQAPGKGLEWVANIRQDGSEKYYVDSVKGRFTISRDNAKNSLYLQMNSLRAEDAAVYYCAREQDTAMVAFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 387 - protein, 461 aa, synthetic construct
DIQMTQSPSSLSASVGDRVTITCQASQDISNYLNWYQQKPGKAPKLLIYDASNLETGVPSRFSGSGSGTDFTLTISSPQPEDLATYYCQQANSFPFTFGQGTKVEIKGGGGSGGGGSGGGGSQVQLVQSGAEVKKPGSSVKVSCKASGYSFTRNDITWVRQANGRGLEWLGRLNPNNGDTVYAEVFQGRVAMTRDTSTDTAYMELASLKSDDTAVYFCARGFWSFDVWGQGTMVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 388 - protein, 344 aa, synthetic construct
QVQLVQSGGGLVQPGGSLRLSCAASSFAFSAYEMSWVRQAPGQRLEWVSGITGSGGTTYYEDSVKGRFTISRDNSKNTLYLQMNTLRAEDTAMYYCVSHLGGTYGSGFSWGQGTLVTVSSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 389 - protein, 339 aa, synthetic construct
QVQLVQSGGGLVQPGGSLRLSCAASDFAFDYYEMSWVRQAPGQGLEWVSSISGSGGTTLYADSVKGRFTISRDNSKNTLYLQMNTLRAEDTALYYCATEDPSSVYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 390 - protein, 347 aa, synthetic construct
QVQLVQSGGGLVQPGGSLRLSCAASSFSFSDYEMSWVRQAPGQRLEWVSAISGSGGSTYYADSVKGRFTISRDNSKNTLYLQMNTLRAEDTATYYCVRVPLGHCSSTDCANPYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 391 - protein, 341 aa, synthetic construct
QVQLVQSGGGLVQPGGSLRLSCAASSFDFADYEMSWVRQAPGQRLEWVSTVSGSGGDTWYADSVKGRFTISRDNSKNTLYLQMNTLRAEDTAVYYCAKVGPKWELVDWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 392 - protein, 347 aa, synthetic construct
QVQLVQSGGGLVQPGGSLRLSCAASSFSFSDYEMSWVRQAPGKGLEWVSLISGDGGSTYYADSVKGRFTISRDNSKNTLYLQMNTLRAEDTAMYYCARDPYYYGSGRITKLDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 393 - protein, 342 aa, synthetic construct
QVQLVQSGGGLVQPGGSLRLSCAASGFSFSTYEMSWVRQAPGQRLEWLSGIIGNSGRTYYSDSVKGRFTISRDNSKNTLYLQMNTLRAEDTAMYYCARSRGGFDAFDIWGQGTMVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 394 - protein, 340 aa, synthetic construct
QVQLVQSGGGLVQPGGSLRLSCAASDFSFASYEMSWVRQAPGQGLEWVALIDNGGKTYYADSVKGRFTISRDNSKNTLYLQMNTLRAEDAALYYCVRDGGVAGPPTWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 395 - protein, 345 aa, synthetic construct
QVQLVQSGGGLVQPGGSLRLSCAASDFSFSYYEMSWVRQAPGKGLEWVSTISGSGDSTYYADSVKGRFTISRDNTKNTLYLQMNTLRAEDTATYYCMTDGETWGTHRYTVYWGQGALVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 396 - protein, 343 aa, synthetic construct
QVQLVQSGGGLVQPGGSLRLSCAASDFSFYFYEMRWVRQAPGQGLEWVSTISGSGDSTYYADSVKGRFTISRDNSKNTLYLQMNTLRAEDTAVYYCARDPEVGGITGEYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 397 - protein, 341 aa, synthetic construct
QVQLVQSGGGLVQPGGSLRLSCAASAFDFASYEMSWVRQAPGQRLEWVSGISGSGGSTYYADSVKGRFTISRDNSKNTLYLQMNTLRAEDTALYYCVKHTIRGVPEVWGQGTMVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 398 - protein, 349 aa, synthetic construct
QVQLVQSGGGLVQPGGSLRLSCAASYFDFDDYEMSWVRQAPGKGLEWVAVISGGGRDTWYADSVKGRFTISRDNSKNTLYLQMNTLRAEDTAVYYCARDPYYQVDDKSAYGAFDIWGQGTAVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 399 - protein, 339 aa, synthetic construct
QVQLVQSGGGLVQPGGSLRLSCAASAFDFYDYEMSWVRQAPGQGLEWVSAISNSGDATYYADSVKGRFTISRDNSKNTLYLQMNTLRAEDTAVYYCTTIEDTLPSWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 400 - protein, 344 aa, synthetic construct
QVQLVQSGGGLVQPGGSLRLSCAASSFDFSSYEMSWVRQAPGQGLEWVSLISGSGDSTYYADSVKGRFTISRDNSKNTLYLQMNTLRAEDTALYYCARDPYYGGEYYFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 401 - protein, 342 aa, synthetic construct
QVQLVQSGGGLVQPGGSLRLSCAASAFSFSSYEMSWVRQAPGQRLEWVAHINGSGGSTYYADSVKGRFTISRDNSKNTLYLQMNTLRAEDTATYYCASSYSSSSQFEYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 402 - protein, 344 aa, synthetic construct
QVQLVQSGGGLVQPGGSLRLSCAASDFSFADYEMSWVRQAPGQGLEWVSLISGDGGSTYYADSVKGRFTISRDNSKNTLYLQMNTLRAEDTAVYYCARDPDSGSYWGGDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 403 - protein, 337 aa, synthetic construct
QVQLVQSGGGLVQPGGSLRLSCAASDFYFADYEMSWVRQAPGKGLEWVSLISGDGGSTYYADSVKGRFTISRDNSKNTLYLQMNTLRAEDTATYYCARDPWDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 404 - protein, 342 aa, synthetic construct
QVQLVQSGGGLVQPGGSLRLSCAASAFDFDSYEMSWVRQAPGQRLEWVSTISGHGGNTYYADSVKGRFTISRDNSKNTLYLQMNTLRAEDTAVYYCVRGDEAEMSTIRWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 405 - protein, 343 aa, synthetic construct
QVQLVQSGGGLVQPGGSLRLSCAASDFDFDAYEMSWVRQAPGQGLEWVSVIYSDGSTYYADSVKGRFTISRDNSKNTLYLQMNTLRAEDTAIYYCAREKDNGDYGDFESWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 406 - protein, 345 aa, synthetic construct
QVQLVQSGGGLVQPGGSLRLSCAASSFAFSDYEMSWVRQAPGQGLEWVSTISGSGGSTYYADSVKGRFTISRDNSKNTLYLQMNTLRAEDTAVYYCARDPADYDILTGGDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 407 - protein, 347 aa, synthetic construct
QVQLVQSGGGLVQPGGSLRLSCAASDFDFSDYEMSWVRQAPGKGLEWVSAISDSGDTTYYADSVKGRFTISRDNSKNTLYLQMNTLRAEDTALYYCAKAPYYDILTGDDAFDTWGQGTMVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 408 - protein, 341 aa, synthetic construct
QVQLVQSGGGLVQPGGSLRLSCAASYFDFDAYEMSWVRQAPGKGLEWVSLISGDGGSTYYADSVKGRFTISRDNSKNTLYLQMNTLRAEDTAVYFCVRDSGYSSFHWGQGTQVTVSSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 409 - protein, 344 aa, synthetic construct
QVQLVQSGGGLVQPGGSLRLSCAASYFDFAYYEMSWVRQAPGKGLEWVSLISGDGGSTYYADSVKGRFTISRDNSKNTLYLQMNTLRAEDTAVYYCARDPYYGGEYYFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 410 - protein, 341 aa, synthetic construct
QVQLVQSGGGLVQPGGSLRLSCAASAFAFSSYEMSWVRQAPGKGLEWVSLISGDGGSTYYADSVKGRFTISRDNTKNTLYLQMNTLRAEDTAVYYCVRETESQDVVYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 411 - protein, 346 aa, synthetic construct
QVQLVQSGGGLVQPGGSLRLSCAASDFDFSDYEMSWVRQAPGKGLEWVSGIYSDGGTYYSDSVKGRFSISRDNSKNTLYLQMNTLRAEDTAVYYCARDPYYYGSGNLRFNDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 412 - protein, 343 aa, synthetic construct
QVQLVQSGGGLVQPGGSLRLSCAASSFDFDDYEMSWVRQAPGKGLEWVSLISGDGGSTYYADSVKGRFTISRDNSKNTLYLQMNTLRAEDTAVYYCARDPFANQQGGDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 413 - protein, 463 aa, synthetic construct
DIQMTQSPSTLSASVGDRVTITCRASQSISSWLAWYQQKPGKAPKLLIYTASSLQSGVPSRFSGSESGTDFTLTISSLQPEDFATYYCQQSYSTPLTFGGGTKVDIKGGGGSGGGGSGGGGSQVQLVQSGAEVKKPGASVKVSCKASGYTFTGYYMHWVRQAPGQGLEWMGWINPNSGGTNYAQKFQGRVTLTRDTSITTAYLELTSLTSDDTAVYYCARGDTGDFFDHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 414 - protein, 463 aa, synthetic construct
DIQMTQSPSSLSASVGDRVTTTCRASQSISSYLNWYQQKPGKAPKLLIYAASSLQSGVPSRFSGSGSGTDFTLTISSLQPEDFATYYCQQSYSTPITFGQGTRLEIKGGGGSGGGGSGGGGSQVQLVQSGAEVKKPGASVKVSCKASGYTFTGYYMHWVRQAPGQGLEWMGWMNPSSGNTGYAHEFQGRVTMTRNTSTGTAYMELSGLRSEDTAVYYCARGDTGDSFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 415 - protein, 472 aa, synthetic construct
QSVLTQPPSVSAAPGQKVTISCSGSSSNIGNNYVSWYQQLPGTAPKLLIYDNNKRPSGIPDRFSGSESGTSATLGITGLQTGDEADYYCGTWDSSLSAWVFGGGTQLIILGGGGSGGGGSGGGGSQVQLQQSGPGLVKPSQTLSLTCAISGDSVSSNSAAWNWIRQSPSRGLEWLGRTHYRSKWYNDYAVSVKSRITISADTSKNQFSLQLNSVTPEDTAVYYCARDRYTVRPDAFDIWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 416 - protein, 466 aa, synthetic construct
QSVLTQPASVSGSLGQSITISCTGTSRDVGGYNFVSWYQQHPGKAPKLIIFDVTTRPSGLSDRFSASKSGNTASLTISGLQAEDEADYFCGSYTASDTMLFGGGTKLTVLGGGGSGGGGSGGGGSQVQLLESGGGVVQPGRSLRLSCAASGFTFDDYAMHWVRQAPGKGLEWVSGISWNSGSIGYADSVKGRFTVSRDNSKNTLYQQMNGLRAEDTAVYYCAREGRPTAFDYWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 417 - protein, 464 aa, synthetic construct
EIVLTQSPATLSLSPGERATLSCRASQSVSSYLAWYQQKPGQAPRLLIYDASNRATGIPDRFSGSGSGTDFTLTISRLEPEDFAVYYCQQYGSSPVTFGQGTKLEIKGGGGSGGGGSGGGGSQVQLVQSGAEVKKPGSSVKVSCKASGGTFSSYAISWVRQAPGQGLEWMGGIIPIFGTANYAQKFQGRVIMTTDTSTSTAHMELRSLRSDDTAVYYCARDADEFGAFDIWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 418 - protein, 108 aa, synthetic construct
DVVMTQSPATLSLSPGEGATLSCRASQSVSTYLAWYQHKPGQSPRLLVFDASNRATGTPARFSGSGSGTDFTLTIDSLEPEDFAVYYCQHRTNWPMYTFGQGTKLEIK

SEQ ID NO: 419 - protein, 6 aa, synthetic construct
QSVSTY

SEQ ID NO: 420 - skipped

SEQ ID NO: 421 - protein, 10 aa, synthetic construct
QHRTNWPMYT

SEQ ID NO: 422 - protein, 121 aa, synthetic construct
EVQLVESGGGLAQPGMSLRLSCVASGFTFDDYAMHWVRQVPGKGLEWVSGISWNSGSTGYADSVKGRFSISRDNAKNSLYLQINSLRPEDTALYYCVRDRAAMAVEHFQKWGQGTPVTVSS

SEQ ID NO: 423 - protein, 8 aa, synthetic construct
GFTFDDYA

SEQ ID NO: 424 - protein, 8 aa, synthetic construct
ISWNSGST

SEQ ID NO: 425 - protein, 14 aa, synthetic construct
VRDRAAMAVEHFQK

SEQ ID NO: 426 - protein, 113 aa, synthetic construct
EIVMTQTPDSLAVSLGERATINCKSSQSVLYRSNNKNYLAWYQQKPGQPPKLLIHWASTRESGVPDRFSGSGSGTDFTLTIGSLQAEDVAIYYCQQYSTTPWTFGQGTKLEIK

SEQ ID NO: 427 - protein, 12 aa, synthetic construct
QSVLYRSNNKNY

SEQ ID NO: 428 - skipped

SEQ ID NO: 429 - protein, 9 aa, synthetic construct
QQYSTTPWT

SEQ ID NO: 430 - protein, 122 aa, synthetic construct
ETQLVESGGGVVQPGRSLSLSCAASGFTFSSYGMHWVRQAPGKGLEWVAVISYEGSTKYYADSVKGRFTISRDNSENTLYLQMNSLRAEDTALYYCTKESITFAGPNYFDFWGQGTLVTVSS

SEQ ID NO: 431 - protein, 8 aa, synthetic construct
GFTFSSYG

SEQ ID NO: 432 - protein, 8 aa, synthetic construct
ISYEGSTK

SEQ ID NO: 433 - protein, 15 aa, synthetic construct
TKESITFAGPNYFDF

SEQ ID NO: 434 - protein, 111 aa, synthetic construct
DVVMTQSPLSLSVTPGQPASISCKSTQSLLHSDGKTYLYWYLQKPGQPPQLLIYEVSNRFSGVPDRFSGSGSGTDFTLKISRVEAEDVGVYYCMQSIQLQTFGQGTKVDIK

SEQ ID NO: 435 - protein, 11 aa, synthetic construct
QSLLHSDGKTY

SEQ ID NO: 436 - skipped

SEQ ID NO: 437 - protein, 8 aa, synthetic construct
MQSIQLQT

SEQ ID NO: 438 - protein, 124 aa, synthetic construct
QMQLVQSGSELKKPGASVKVSCKTSGYTFMTYAMNWVRQAPGQGLEWMGWINTNTGKPTYDQGFTGRFVFSLDTSVSTAYLQINNLKAEDTAVYYCARGDEYSGYVRLDGVDYWGQGTLVTVSS

SEQ ID NO: 439 - protein, 8 aa, synthetic construct
GYTFMTYA

SEQ ID NO: 440 - protein, 8 aa, synthetic construct
INTNTGKP

SEQ ID NO: 441 - protein, 17 aa, synthetic construct
ARGDEYSGYVRLDGVDY

SEQ ID NO: 442 - protein, 107 aa, synthetic construct
EIVMTQSPATLSLSPGERATLSCRASQSVSNYLAWYQQKPGQAPRLLIYDASNRATGIPARFSGRGSGTDFTLTISSLEPEDFAVYYCQQRSDWPLTFGGGTKVEIK

SEQ ID NO: 443 - protein, 6 aa, synthetic construct
QSVSNY

SEQ ID NO: 444 - skipped

SEQ ID NO: 445 - protein, 9 aa, synthetic construct
QQRSDWPLT

SEQ ID NO: 446 - protein, 122 aa, synthetic construct
QVQLVQSGAEVKKPGESLRISCKGSGYSFTNYWISWVRQMPGKGLEWMGRIDPSDSYTNYSPSFQGHVTISADKSISTAYLQWSSLKASDTAMYYCARLGSGWLQYSANDYWGQGTLVTVSS

SEQ ID NO: 447 - protein, 8 aa, synthetic construct
GYSFTNYW

SEQ ID NO: 448 - protein, 8 aa, synthetic construct
IDPSDSYT

SEQ ID NO: 449 - protein, 15 aa, synthetic construct
ARLGSGWLQYSANDY

SEQ ID NO: 450 - protein, 107 aa, synthetic construct
AIQLTQSPPSLSASVGDRVTISCRASQRITTFLNWYQQKPGKAPRLLIYAASSLQSGVPPRFSGSGSGTVFTLTISSLHPEDFATYYCQQSYGTPWTFGQGTKVEIK

SEQ ID NO: 451 - protein, 6 aa, synthetic construct
QRITTF

SEQ ID NO: 452 - skipped

SEQ ID NO: 453 - protein, 9 aa, synthetic construct
QQSYGTPWT

SEQ ID NO: 454 - protein, 125 aa, synthetic construct
EVQLLESGGGVVQPGRSLRLSCAASGVTSSSYGIHWVRQAPGKGLEWLAVLLNDGTFEDSRDSVQGRFTISRDKVKNTVSLQMNRLTTEDTAVYYCATTPHYDDMTGFFSPFDYWGQGTLVTVSS

SEQ ID NO: 455 - protein, 8 aa, synthetic construct
GVTSSSYG

SEQ ID NO: 456 - protein, 8 aa, synthetic construct
LLNDGTFE

SEQ ID NO: 457 - protein, 18 aa, synthetic construct
ATTPHYDDMTGFFSPFDY

SEQ ID NO: 458 - protein, 107 aa, synthetic construct
DIVMTQSPDSLAVSLGERATINCKSSQNVYNSLTWYQQQPGQSPKVLLYWASTRESGVPDRFSGSGSGTDFTLTISSLQAEDVAVYYCQQYYSLPLTFGGGTKVEIK

SEQ ID NO: 459 - protein, 6 aa, synthetic construct
QNVYNS

SEQ ID NO: 460 - skipped

SEQ ID NO: 461 - protein, 9 aa, synthetic construct
QQYYSLPLT

SEQ ID NO: 462 - protein, 121 aa, synthetic construct
QVQLVESGGGVVQPGTSLRLSCAASGGTFSTFGIHWVRQAPGKGLEWVAVISYDGSTKYYADSVKGRFTISRDNSKNTVFVQMNSLRAEDTAVYYCGKGLEATIMTHMDVWGQGPLVTVSS

SEQ ID NO: 463 - protein, 8 aa, synthetic construct
GGTFSTFG

SEQ ID NO: 464 - protein, 8 aa, synthetic construct
ISYDGSTK

SEQ ID NO: 465 - protein, 14 aa, synthetic construct
GKGLEATIMTHMDV

SEQ ID NO: 466 - protein, 107 aa, synthetic construct
DVVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIK

SEQ ID NO: 467 - protein, 6 aa, synthetic construct
QSVSSN

SEQ ID NO: 468 - skipped

SEQ ID NO: 469 - protein, 9 aa, synthetic construct
QQYNNWPRT

SEQ ID NO: 470 - protein, 128 aa, synthetic construct
QITLKESGPALVKPTQTLTLTCTFSGFSLSTSGMCVSWIRQPPGKALEWLALIDWDDDKYYSTSLKTRLTISKDTSKNQVVLTMTNMDPVDTATYYCARIRAYYDSSGYYYWYWFDPWGQGTLVTVSS

SEQ ID NO: 471 - protein, 10 aa, synthetic construct
GFSLSTSGMC

SEQ ID NO: 472 - protein, 7 aa, synthetic construct
IDWDDDK

SEQ ID NO: 473 - protein, 20 aa, synthetic construct
ARIRAYYDSSGYYYWYWFDP

SEQ ID NO: 474 - protein, 107 aa, synthetic construct
EIVLTQSPATLSVSPGERATLSCRASQSVSNNVAWYQQKPGQAPRLLLYGASTRASGIPDRLTGSGSGTEFTLTFSSLQSEDFAVYYCQQYDKWPFTFGPGTKLEIK

SEQ ID NO: 475 - protein, 6 aa, synthetic construct
QSVSNN

SEQ ID NO: 476 - skipped

SEQ ID NO: 477 - protein, 9 aa, synthetic construct
QQYDKWPFT

SEQ ID NO: 478 - protein, 134 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREGPPYYDFWSGYLLADHYGMDVWGQGTTVTVSS

SEQ ID NO: 479 - protein, 10 aa, synthetic construct
GDSVSSNSAA

SEQ ID NO: 480 - protein, 9 aa, synthetic construct
TYYRSKWYN

SEQ ID NO: 481 - protein, 24 aa, synthetic construct
AREGPPYYDFWSGYLLADHYGMDV

SEQ ID NO: 482 - protein, 114 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPRFTFGPGTKVEIK

SEQ ID NO: 483 - protein, 12 aa, synthetic construct
QSVLYSSNNKNY

SEQ ID NO: 484 - skipped

SEQ ID NO: 485 - protein, 10 aa, synthetic construct
QQYYSTPRFT

SEQ ID NO: 486 - protein, 121 aa, synthetic construct
QVQLLESGGGVVQPGRSLRLSCAASGFTFSTYGMFWVRQAPGKGLEWVAAISYDGSNTYYADFVRGQFSISRDNPKNTLYLQMNSLRAEDTAVYYCARGGIRTVMYYFDYWGQGTLVTVSS

SEQ ID NO: 487 - protein, 8 aa, synthetic construct
GFTFSTYG

SEQ ID NO: 488 - protein, 8 aa, synthetic construct
ISYDGSNT

SEQ ID NO: 489 - protein, 14 aa, synthetic construct
ARGGIRTVMYYFDY

SEQ ID NO: 490 - protein, 113 aa, synthetic construct
DIVMTQSPDSLTVSLGERATINCKSSQNLLYSSDNNNYLAWFQQKPGQPPKLLIHWASTRESGVPDRFSGSGSGTEFTLTISSLQAEDVAVYYCQQYYSTPLTFGGGTKLEIK

SEQ ID NO: 491 - protein, 12 aa, synthetic construct
QNLLYSSDNNNY

SEQ ID NO: 492 - skipped

SEQ ID NO: 493 - protein, 9 aa, synthetic construct
QQYYSTPLT

SEQ ID NO: 494 - protein, 117 aa, synthetic construct
EVQLVESGGGLVQPGGSLRLSCAASGITFSSHWMHWVRQAPGKGPVWVSRINGDGSSTTYVDSVKGRFSISRDNAKNTLYLQMNSLRAEDTAVYYCVRDCGDYGCYWGQGTLVTVSS

SEQ ID NO: 495 - protein, 8 aa, synthetic construct
GITFSSHW

SEQ ID NO: 496 - protein, 8 aa, synthetic construct
INGDGSST

SEQ ID NO: 497 - protein, 10 aa, synthetic construct
VRDCGDYGCY

SEQ ID NO: 498 - protein, 108 aa, synthetic construct
DVVMTQSPATLSLSLGERATLSCRASRSVNSNLAWYQQKPGQAPRLLIYDASTRVTGISERFSGSGSGIEFTLTISSLQSEDSAVYYCQQYNDWPPITLGQGTKVEIK

SEQ ID NO: 499 - protein, 6 aa, synthetic construct
RSVNSN

SEQ ID NO: 500 - skipped

SEQ ID NO: 501 - protein, 10 aa, synthetic construct
QQYNDWPPIT

SEQ ID NO: 502 - protein, 147 aa, synthetic construct
EVQLVQSGAEVKKPGASVKVSCKASGYTFTGYYMHWVRQAPGQGLEWMGGINPNSGGTNYAQKFQGRVTMTRDTSISTAYMELSRLRSDDTAVYYCARSRIYWSYDILTGYSADTWELEQKIRPGEYYYYYYGMDVWGQGTTVTVSS

SEQ ID NO: 503 - protein, 8 aa, synthetic construct
GYTFTGYY

SEQ ID NO: 504 - protein, 8 aa, synthetic construct
INPNSGGT

SEQ ID NO: 505 - protein, 40 aa, synthetic construct
ARSRIYWSYDILTGYSADTWELEQKIRPGEYYYYYYGMDV

SEQ ID NO: 506 - protein, 113 aa, synthetic construct
DIVMTQSPDSLAVSLGERATINCMSSQSVLYSSNNKNYLAWYQQKPGQPPKLLIYWASTRESGVPDRFSGSGSRTHFTLTISSLQAEDVAAYYCQQYYSTLPTFGGGTKVEIK

SEQ ID NO: 507 - protein, 12 aa, synthetic construct
QSVLYSSNNKNY

SEQ ID NO: 508 - skipped

SEQ ID NO: 509 - protein, 9 aa, synthetic construct
QQYYSTLPT

SEQ ID NO: 510 - protein, 121 aa, synthetic construct
ETQLVESGGGLVKPGGSLRVSCAVSGFNFNNAWMNWVRQTPGKGLEWVGQIKTTPEGATTDYPAPVKGRFTISRDDSKNTLSLQMASLTTEDTAIYYCHTDDHNWNPHDHWGQGTLVTVSS

SEQ ID NO: 511 - protein, 8 aa, synthetic construct
GFNFNNAW

SEQ ID NO: 512 - protein, 10 aa, synthetic construct
IKTTPEGATT

SEQ ID NO: 513 - protein, 12 aa, synthetic construct
HTDDHNWNPHDH

SEQ ID NO: 514 - protein, 112 aa, synthetic construct
ETTLTQSPDSLAVSLGERATINCKSSQSVLYSSNNKNYLAWYQQKPGQPPKLLIYWASTRESGVPDRFSGSGSGTDFTLTISSLQAGDVAIYYCQQYYTTPTFGQGTRLEIK

SEQ ID NO: 515 - protein, 12 aa, synthetic construct
QSVLYSSNNKNY

SEQ ID NO: 516 - skipped

SEQ ID NO: 517 - protein, 8 aa, synthetic construct
QQYYTTPT

SEQ ID NO: 518 - protein, 124 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCAGGATSGSYYAGFGGCYWGQGTLVTVSS

SEQ ID NO: 519 - protein, 8 aa, synthetic construct
GGTFSSYA

SEQ ID NO: 520 - protein, 8 aa, synthetic construct
IIPIFGTA

SEQ ID NO: 521 - protein, 17 aa, synthetic construct
AGGATSGSYYAGFGGCY

SEQ ID NO: 522 - protein, 107 aa, synthetic construct
EIVMTQSPATLSLSPGERATLFCTASQSVGNFLTWYQQKPGQPPRLLIYDASNRAAGVPARFSGSGFGTDFTLTISTLEPDDFAVYYCQQRRDWPLTFGGGTKVEIK

SEQ ID NO: 523 - protein, 6 aa, synthetic construct
QSVGNF

SEQ ID NO: 524 - skipped

SEQ ID NO: 525 - protein, 9 aa, synthetic construct
QQRRDWPLT

SEQ ID NO: 526 - protein, 120 aa, synthetic construct
ETQLVESGAEVKEPGASVKISCETSEYTFTNYFFHWVRQTPGHGLEWMGIINPYDAITTSAQKFQGRVTLTWDTSTGTVYMDLNSLRFEDTATYYCARASWRDVPLFDYWGQGTLVTVSS

SEQ ID NO: 527 - protein, 8 aa, synthetic construct
EYTFTNYF

SEQ ID NO: 528 - protein, 8 aa, synthetic construct
INPYDAIT

SEQ ID NO: 529 - protein, 13 aa, synthetic construct
ARASWRDVPLFDY

SEQ ID NO: 530 - protein, 107 aa, synthetic construct
NIQMTQSPSSLSASVGDRVTITCRASQGISKNLGWYQQKPGKAPKRLIYAASFLQSGVPSRFSGSGSGTDFTLTISSLQPEDFATYYCLQHNTFPRTFGQGTKVEIK

SEQ ID NO: 531 - protein, 6 aa, synthetic construct
QGISKN

SEQ ID NO: 532 - skipped

SEQ ID NO: 533 - protein, 9 aa, synthetic construct
LQHNTFPRT

SEQ ID NO: 534 - protein, 122 aa, synthetic construct
QVQLVQSGAEVKKPGESLKISCKGSGYGFTSYWIGWVRQMPGKGLEWMGIIYPGDSDTRYSPSFQGQVTISADKSISTAYLQWSSLKASDTAMYYCARGGYYYGPGRAFDIWGQGTMVTVSS

SEQ ID NO: 535 - protein, 8 aa, synthetic construct
GYGFTSYW

SEQ ID NO: 536 - protein, 8 aa, synthetic construct
IYPGDSDT

SEQ ID NO: 537 - protein, 15 aa, synthetic construct
ARGGYYYGPGRAFDI

SEQ ID NO: 538 - protein, 107 aa, synthetic construct
ETTLTQSPAFMSATPGDKVNISCKASQDIDDDMNWYQQKPGEAAIFIIQEATTLVPGIPPRFSGSGYGTDFTLTINNIESEDAAYYFCLQHDNFPRTFGQGTRLEIK

SEQ ID NO: 539 - protein, 6 aa, synthetic construct
QDIDDD

SEQ ID NO: 540 - skipped

SEQ ID NO: 541 - protein, 9 aa, synthetic construct
LQHDNFPRT

SEQ ID NO: 542 - protein, 119 aa, synthetic construct
EVQLVQSGPEVEKPGASVMLSCKSSGYTFSNYYVHWVRQAPGQGVEWMAVITPSGDSAFFAQRFRGRLTTTRDTSTSTVYMELSSLTSEDTALYYCASESPGSGGLENWGQGTLVTVSS

SEQ ID NO: 543 - protein, 8 aa, synthetic construct
GYTFSNYY

SEQ ID NO: 544 - protein, 8 aa, synthetic construct
ITPSGDSA

SEQ ID NO: 545 - protein, 12 aa, synthetic construct
ASESPGSGGLEN

SEQ ID NO: 546 - protein, 112 aa, synthetic construct
DVVMTQSPLSLPVTLGQPASISCRSSHSLVYSDGNTYLNWFQQRPGQSPRRLIYKVSNWDSGVPDRFSGSGSGTDFTLKISRVEAEDVGVYYCMQGTHWPPTFGQGTRLEIK

SEQ ID NO: 547 - protein, 11 aa, synthetic construct
HSLVYSDGNTY

SEQ ID NO: 548 - skipped

SEQ ID NO: 549 - protein, 9 aa, synthetic construct
MQGTHWPPT

SEQ ID NO: 550 - protein, 122 aa, synthetic construct
ETQLVESGGGLVQPGGSLRLSCTASGFTFSNYWMNWVRQAPGKGLEWVANMKQDGSDKYYANSVKGRFAISRDNARNSLFLQMNSLRAEDTAVYFCARAGRDYFGLGTPDFWGQGTLVTVSS

SEQ ID NO: 551 - protein, 8 aa, synthetic construct
GFTFSNYW

SEQ ID NO: 552 - protein, 8 aa, synthetic construct
MKQDGSDK

SEQ ID NO: 553 - protein, 15 aa, synthetic construct
ARAGRDYFGLGTPDF

SEQ ID NO: 554 - protein, 109 aa, synthetic construct
DVVMTQSPGTLSLSPGERATLSCRASQSVSSSYLAWYQQKPGQAPRLLIYGASSRATGIPDRFSGSGSGTDFTLTISRLEPEDFAVYYCQQYGSSSMYTFGQGTKVDIK

SEQ ID NO: 555 - protein, 7 aa, synthetic construct
QSVSSSY

SEQ ID NO: 556 - skipped

SEQ ID NO: 557 - protein, 10 aa, synthetic construct
QQYGSSSMYT

SEQ ID NO: 558 - protein, 124 aa, synthetic construct
QVQLMQSGAEVKKPGASAKVSCKASGYQFNGYYIYWVRQAPGQGLEWMGWINAYSGDTSYPQTFQGRVTMTRDTSIDTAYMELKSLRSDDTAVYYCARGRFYDFWSGYHHLDYWGQGTLVTVSS

SEQ ID NO: 559 - protein, 8 aa, synthetic construct
GYQFNGYY

SEQ ID NO: 560 - protein, 8 aa, synthetic construct
INAYSGDT

SEQ ID NO: 561 - protein, 17 aa, synthetic construct
ARGRFYDFWSGYHHLDY

SEQ ID NO: 562 - protein, 106 aa, synthetic construct
DVVMTQSPATLSLSPGERATLSCRASQSVNSHLAWYQQKPGQAPRLLIYEASNRATGIPARFSGSGSGTDFTLTISSLEPEDFAVYYCQQRKNWPPFGQGTRLEIK

SEQ ID NO: 563 - protein, 6 aa, synthetic construct
QSVNSH

SEQ ID NO: 564 - skipped

SEQ ID NO: 565 - protein, 8 aa, synthetic construct
QQRKNWPP

SEQ ID NO: 566 - protein, 123 aa, synthetic construct
ETQLVESGGGLVQPGGSLRLSCAASGFTFSSYEMNWVRQAPGKGLEWVSYISSSVSTIYYADSVKGRFTISRDNAKNSLYLQMNSLRAEDTAIYYCARGGSRGAVAGREFDYWGQGTLVTVSS

SEQ ID NO: 567 - protein, 8 aa, synthetic construct
GFTFSSYE

SEQ ID NO: 568 - protein, 8 aa, synthetic construct
ISSSVSTI

SEQ ID NO: 569 - protein, 16 aa, synthetic construct
ARGGSRGAVAGREFDY

SEQ ID NO: 570 - protein, 112 aa, synthetic construct
QSALTQPASVSGSPGQSITISCTGTSGDVGGYDFVSWYQQHPGKAPKVLIYDVSERPSGVSTRFSGSKSGNTASLTISGLQAEDEADYYCTSYTTSITPLYVFGTGTKVTVL

SEQ ID NO: 571 - protein, 9 aa, synthetic construct
SGDVGGYDF

SEQ ID NO: 572 - skipped

SEQ ID NO: 573 - protein, 12 aa, synthetic construct
TSYTTSITPLYV

SEQ ID NO: 574 - protein, 124 aa, synthetic construct
EVQLVESGGEAKMPGGSVKISCKSSGYTFTEYDIAWVRQAPGQGLEWLGWINAGNGDTKSAHDFQGRVTLTTDTSTSTAYMELRSLKSDDTAVYFCARRAWLQLWPHGYYFDYWGQGALVTVSS

SEQ ID NO: 575 - protein, 8 aa, synthetic construct
GYTFTEYD

SEQ ID NO: 576 - protein, 8 aa, synthetic construct
INAGNGDT

SEQ ID NO: 577 - protein, 17 aa, synthetic construct
ARRAWLQLWPHGYYFDY

SEQ ID NO: 578 - protein, 110 aa, synthetic construct
QSALTQPPAASGTPGQRVTIFCSGSRSSIGRNTVNWYQQFAGEAPKLLIEKNNQRPWGVPDRFSASKSGTSASLAISGLQSEDEAHYFCATWDDSLPGPVFGGGTKVTVL

SEQ ID NO: 579 - protein, 8 aa, synthetic construct
RSSIGRNT

SEQ ID NO: 580 - skipped

SEQ ID NO: 581 - protein, 11 aa, synthetic construct
ATWDDSLPGPV

SEQ ID NO: 582 - protein, 122 aa, synthetic construct
EVQLVESGGGLVKPGGSLRLSCAASGFTFSSYSMNWVRLAPGKGLEWVAFISATTTNILYADSLKGRFTISRDNAKNSLDLHINSLRADDTAVYYCARVDRTVRTYYYMDVWGPGTTVTVSS

SEQ ID NO: 583 - protein, 8 aa, synthetic construct
GFTFSSYS

SEQ ID NO: 584 - protein, 8 aa, synthetic construct
ISATTTNI

SEQ ID NO: 585 - protein, 15 aa, synthetic construct
ARVDRTVRTYYYMDV

SEQ ID NO: 586 - protein, 110 aa, synthetic construct
QSALTQPASVSGSPGQSITISCAGTSSDVGGYNYVSWYQQHPGKAPRLIIYDVSNRPSGVSDRFSGSKSGNTASLTISGLQADDEADYYCSSYRNSSTVVFGPATKLTVL

SEQ ID NO: 587 - protein, 9 aa, synthetic construct
SSDVGGYNY

SEQ ID NO: 588 - skipped

SEQ ID NO: 589 - protein, 10 aa, synthetic construct
SSYRNSSTVV

SEQ ID NO: 590 - protein, 119 aa, synthetic construct
QVQLVESGGGLVQPGGSLRLSCAASGFTFSTYSMNWVRQAPGKGLEWVSYISSSSSGRYYADSVKGRFTISRDNAKNSLYLQMNSLRDEDTAVYYCARDESGLVPCDYWGQGTLVTVSS

SEQ ID NO: 591 - protein, 8 aa, synthetic construct
GFTFSTYS

SEQ ID NO: 592 - protein, 8 aa, synthetic construct
ISSSSSGR

SEQ ID NO: 593 - protein, 12 aa, synthetic construct
ARDESGLVPCDY

SEQ ID NO: 594 - protein, 110 aa, synthetic construct
QSALTQPASVAGSPGQSITISCTGTSSDVGGYNWVSWYQHNPHKAPKLIIFDVTQRPSGVSNRFSGSKSGNTASLTISGLQTEDEADYYCTSYTTRSTLVFGGGTKLTVL

SEQ ID NO: 595 - protein, 9 aa, synthetic construct
SSDVGGYNW

SEQ ID NO: 596 - skipped

SEQ ID NO: 597 - protein, 10 aa, synthetic construct
TSYTTRSTLV

SEQ ID NO: 598 - protein, 118 aa, synthetic construct
QVQLQQSGGGVVQPGRSLRLSCAASGFTFSSYAMHWVRQAPGKGLEWVAVISYDGSNKYYADSVKGRFTISRDNSKNTLYLQMNSLRAEDTAVYYCAREVVGATLDYWGQGTLVTVSS

SEQ ID NO: 599 - protein, 8 aa, synthetic construct
GFTFSSYA

SEQ ID NO: 600 - protein, 8 aa, synthetic construct
ISYDGSNK

SEQ ID NO: 601 - protein, 11 aa, synthetic construct
AREVVGATLDY

SEQ ID NO: 602 - protein, 106 aa, synthetic construct
SYELTQLPSVSVSPGQTARITCSGDALPKQYAYWYQQKPGQAPVLVIYKDSERPSGIPERFSGSSSGTTVTLTISGVQAEDEADYYCQSADSSGTCFGGGTKLTVL

SEQ ID NO: 603 - protein, 6 aa, synthetic construct
ALPKQY

SEQ ID NO: 604 - skipped

SEQ ID NO: 605 - protein, 9 aa, synthetic construct
QSADSSGTC

SEQ ID NO: 606 - protein, 135 aa, synthetic construct
QVQLQQSGPGLVKPSQTLSLTCDISGDSVSSNSAAWNWIRQSPSRGLEWLGRTYYRSKWYNDYAISVQSRITINPDTSKNQFSLQLKSVSPEDTAVYYCARAGGPHQTILVAGRVRYYYYGMDVWGQGTTVTVSS

SEQ ID NO: 607 - protein, 10 aa, synthetic construct
GDSVSSNSAA

SEQ ID NO: 608 - protein, 9 aa, synthetic construct
TYYRSKWYN

SEQ ID NO: 609 - protein, 25 aa, synthetic construct
ARAGGPHQTILVAGRVRYYYYGMDV

SEQ ID NO: 610 - protein, 110 aa, synthetic construct
SYVLTQPPSASGTPGQRVTISCSGSSSNIGGNTVIWYQQLPGSAPKLLIYINSQRPSGVPARFSGSKSGTSASLAITGLQSEDEADYYCAAWDDSLNARVFGGGTKVTVL

SEQ ID NO: 611 - protein, 8 aa, synthetic construct
SSNIGGNT

SEQ ID NO: 612 - skipped

SEQ ID NO: 613 - protein, 11 aa, synthetic construct
AAWDDSLNARV

SEQ ID NO: 614 - protein, 128 aa, synthetic construct
QVQLVESGAEVKKPGSSVKVSCKASGGTFSSYAISWVRQAPGQGLEWMGGIIPILGIANYAQKFQGRVTITADESTSTAYMELSSLRSEDTAVYYCAREHRDGYNFWSGVYYYGMDVWDQGTTVTVSS

SEQ ID NO: 615 - protein, 8 aa, synthetic construct
GGTFSSYA

SEQ ID NO: 616 - protein, 8 aa, synthetic construct
IIPILGIA

SEQ ID NO: 617 - protein, 21 aa, synthetic construct
AREHRDGYNFWSGVYYYGMDV

SEQ ID NO: 618 - protein, 106 aa, synthetic construct
SYELTQPPSVSVSPGQTASITCSGDNLGDKYACWYQQKPGQSPVLVMYQDNKRPSGIPERFSGSNSGNTATLTISGTQAMDEADYYCQAWDSSTAVFGGGTKVTVL

SEQ ID NO: 619 - protein, 6 aa, synthetic construct
NLGDKY

SEQ ID NO: 620 - skipped

SEQ ID NO: 621 - protein, 9 aa, synthetic construct
QAWDSSTAV

SEQ ID NO: 622 - protein, 124 aa, synthetic construct
QVQLQESGPGLVKPSETLSLTCTVSGDSISSNNYYWGWIRQPPGKGLDWIGFIYYSGSTYYNSSLKSRVTMSVDTSKNQFSLKLSSVTAADTAVYYCARSARVSIPGRGGFDYWGQGTLVTVSS

SEQ ID NO: 623 - protein, 10 aa, synthetic construct
GDSISSNNYY

SEQ ID NO: 624 - protein, 7 aa, synthetic construct
IYYSGST

SEQ ID NO: 625 - protein, 16 aa, synthetic construct
ARSARVSIPGRGGFDY

SEQ ID NO: 626 - protein, 109 aa, synthetic construct
QTVVTQEPSLTVSPGGTVTLTCGSSTGVVTSGHYPYWFQQKPGQAPKTLIFDTTKKYSWTPARFSGSLLGGKAALTLSGAQPEDEADYYCFLSYSGARVFGGGTKLTVL

SEQ ID NO: 627 - protein, 9 aa, synthetic construct
TGVVTSGHY

SEQ ID NO: 628 - skipped

SEQ ID NO: 629 - protein, 9 aa, synthetic construct
FLSYSGARV

SEQ ID NO: 630 - protein, 136 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARDGKKEKKYYYGSGSYRGAPNYFDYWGQGTLVTVSS

SEQ ID NO: 631 - protein, 10 aa, synthetic construct
GDSVSSNSAA

SEQ ID NO: 632 - protein, 9 aa, synthetic construct
TYYRSKWYN

SEQ ID NO: 633 - protein, 26 aa, synthetic construct
ARDGKKEKKYYYGSGSYRGAPNYFDY

SEQ ID NO: 634 - protein, 106 aa, synthetic construct
DIQVTQSPSSLSASVGDRVTITCRASLGIRNDLGWCQQKPGKAPKRLIYDASSLQSGVPSRFSGSGSGTDFTLTISSLQPEDFANYNCLQHSSSLTFGGGTKVEIK

SEQ ID NO: 635 - protein, 6 aa, synthetic construct
LGIRND

SEQ ID NO: 636 - skipped

SEQ ID NO: 637 - protein, 8 aa, synthetic construct
LQHSSSLT

SEQ ID NO: 638 - protein, 125 aa, synthetic construct
RVTLKESGPPRVKPTQTLTLTCTLSGFSISTSGVGVGWIRQPPGKALEWLAFIYWDDDKRYNPSLKSRLTISKDTSKNQVVLTMTNMDPVDTATYFCAHRSRWASSWDLGWFDYWGQGTLVTVSS

SEQ ID NO: 639 - protein, 10 aa, synthetic construct
GFSISTSGVG

SEQ ID NO: 640 - protein, 7 aa, synthetic construct
IYWDDDK

SEQ ID NO: 641 - protein, 17 aa, synthetic construct
AHRSRWASSWDLGWFDY

SEQ ID NO: 642 - protein, 114 aa, synthetic construct
DIVMTQSPDSLAVSLGERATINCKSSQSILYSSNNNNYLAWYQQKPGQPPKLLIYWASTRESGVPDRFSGSGSGTDFTLTISSLQAEDVAVYYCQHYLGTPPWTFGQGTKVDIK

SEQ ID NO: 643 - protein, 12 aa, synthetic construct
QSILYSSNNNNY

SEQ ID NO: 644 - skipped

SEQ ID NO: 645 - protein, 10 aa, synthetic construct
QHYLGTPPWT

SEQ ID NO: 646 - protein, 131 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCASLGYCTNGVCWGPNLLGTPYMDVWGKGTTVTVSS

SEQ ID NO: 647 - protein, 8 aa, synthetic construct
GGTFSSYA

SEQ ID NO: 648 - protein, 8 aa, synthetic construct
IIPIFGTA

SEQ ID NO: 649 - protein, 24 aa, synthetic construct
ASLGYCTNGVCWGPNLLGTPYMDV

SEQ ID NO: 650 - protein, 110 aa, synthetic construct
QSVVTQEPSLSVSPGGTVTLTCGLSSGSVSTNYYASWYQKTPGQAPRTLIYNTDSRSSGVPDRFSGSILGNTAALTITGAQADDESDYYCLLYMGDGFWVFGGGTQLIIL

SEQ ID NO: 651 - protein, 9 aa, synthetic construct
SGSVSTNYY

SEQ ID NO: 652 - skipped

SEQ ID NO: 653 - protein, 10 aa, synthetic construct
LLYMGDGFWV

SEQ ID NO: 654 - protein, 123 aa, synthetic construct
QVQLQESGPGLVKPSQTLSLTCTVSGGSISSGDTYWSWIRQHPGMGLVWIGYISHSGSTYYNPSLKTRVTISVDTSKNQFSLNLSSVTAADTAVYYCARALYYSGGWGYFDYWGQGTLVTVSS

SEQ ID NO: 655 - protein, 10 aa, synthetic construct
GGSISSGDTY

SEQ ID NO: 656 - protein, 7 aa, synthetic construct
ISHSGST

SEQ ID NO: 657 - protein, 15 aa, synthetic construct
ARALYYSGGWGYFDY

SEQ ID NO: 658 - protein, 112 aa, synthetic construct
QAVVTQEPSLTVSPGGTVTLTCASGTGAVTNAYHPNWFQQKPGQAPRPLIYSTNNKYSWTPARFSGSLLGDKAVLTLSGVQPEDEAEYYCLLYFDDPQPLWVFGGGTKLTVL

SEQ ID NO: 659 - protein, 9 aa, synthetic construct
TGAVTNAYH

SEQ ID NO: 660 - skipped

SEQ ID NO: 661 - protein, 12 aa, synthetic construct
LLYFDDPQPLWV

SEQ ID NO: 662 - protein, 122 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAREARPDSYYYYYMDVWGKGTTVTVSS

SEQ ID NO: 663 - protein, 8 aa, synthetic construct
GGSISSYY

SEQ ID NO: 664 - protein, 7 aa, synthetic construct
IYYSGST

SEQ ID NO: 665 - protein, 16 aa, synthetic construct
AREARPDSYYYYYMDV

SEQ ID NO: 666 - protein, 113 aa, synthetic construct
DIVMTQTPLSLPVTPGEPASISCRSSQSLLHSNGYNYLDWYLQKPGQSPQLLIYLGSNRASGVPDRFSGSGSGTDFTLKISRVEAEDVGVYYCMQALQTPPWTFGQGTKLEIK

SEQ ID NO: 667 - protein, 11 aa, synthetic construct
QSLLHSNGYNY

SEQ ID NO: 668 - skipped

SEQ ID NO: 669 - protein, 10 aa, synthetic construct
MQALQTPPWT

SEQ ID NO: 670 - protein, 127 aa, synthetic construct
ETQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAREGLGYYYDSSGSIDYFDYWGQGTLVTVSS

SEQ ID NO: 671 - protein, 8 aa, synthetic construct
GFTFSSYW

SEQ ID NO: 672 - protein, 8 aa, synthetic construct
IKQDGSEK

SEQ ID NO: 673 - protein, 20 aa, synthetic construct
AREGLGYYYDSSGSIDYFDY

SEQ ID NO: 674 - protein, 110 aa, synthetic construct
NFMLTQPHSVSESPGKTVTISCARSSGNIATNYVQWYQQRPGSSPTAVIFEDNQRPSGVPDRFSGSIDSSSNSASLTISGLQTADEADYYCQSYDGSSVIFGGGTQLIIL

SEQ ID NO: 675 - protein, 8 aa, synthetic construct
SGNIATNY

SEQ ID NO: 676 - skipped

SEQ ID NO: 677 - protein, 9 aa, synthetic construct
QSYDGSSVI

SEQ ID NO: 678 - protein, 120 aa, synthetic construct
EVQLVQSGAEVKKPGASVKVSCKASGYTFTGYYMHWVRQAPGQGLEWMGWINPNSGGTNYAQKFQGRVTMTRDTSLNTVYMELRALRVDDTAVYYCAEGHSRRWSPGDYWGQGSLVTVSS

SEQ ID NO: 679 - protein, 8 aa, synthetic construct
GYTFTGYY

SEQ ID NO: 680 - protein, 8 aa, synthetic construct
INPNSGGT

SEQ ID NO: 681 - protein, 13 aa, synthetic construct
AEGHSRRWSPGDY

SEQ ID NO: 682 - protein, 111 aa, synthetic construct
QSVVTQEPSLTVSPGGTVTLTCGSSTGVVTSGHYPYWFQQKPGQAPTTLIYDTSNKHSWTPARFSGSLLGGKAVLTLSGAQPEDEAEYYCLLSFGGAPNWVFGGGTKLTVL

SEQ ID NO: 683 - protein, 9 aa, synthetic construct
TGVVTSGHY

SEQ ID NO: 684 - skipped

SEQ ID NO: 685 - protein, 11 aa, synthetic construct
LLSFGGAPNWV

SEQ ID NO: 686 - protein, 126 aa, synthetic construct
RVTLKESGPVLVKPTETLTLTCTVSGFSLSNARMGVSWIRQPPGKALEWLAHIFSNDEKSYSTSLKSRLTISKDTSKSQVVLTMTNMDPVDTATYYCARVPRGVIIDYYYYGMDVWGQGTTVTVSS

SEQ ID NO: 687 - protein, 10 aa, synthetic construct
GFSLSNARMG

SEQ ID NO: 688 - protein, 7 aa, synthetic construct
IFSNDEK

SEQ ID NO: 689 - protein, 18 aa, synthetic construct
ARVPRGVIIDYYYYGMDV

SEQ ID NO: 690 - protein, 110 aa, synthetic construct
QSALTQPPSVSGSPGQSVTISCTGTSSDVGSYDYVSWYQQHPGTVPKPMIYNVNTQPSGVPDRFSGSKSGNTASMTISGLQAEDEADYQCCSYTSSATYVFGTGTKLTVL

SEQ ID NO: 691 - protein, 9 aa, synthetic construct
SSDVGSYDY

SEQ ID NO: 692 - skipped

SEQ ID NO: 693 - protein, 10 aa, synthetic construct
CSYTSSATYV

SEQ ID NO: 694 - protein, 121 aa, synthetic construct
ETQLVESGPEVKRPGASVKVSCKASNYPFTTYGITWVRQAPGQGLEWMGWISTYKGNTNYAQRLQGRVTMTVETFTSTAYMELKNLTSDDTAIYYCARGRTTLLHGEYLVWGQGPLVTVSS

SEQ ID NO: 695 - protein, 8 aa, synthetic construct
NYPFTTYG

SEQ ID NO: 696 - protein, 8 aa, synthetic construct
ISTYKGNT

SEQ ID NO: 697 - protein, 14 aa, synthetic construct
ARGRTTLLHGEYLV

SEQ ID NO: 698 - protein, 110 aa, synthetic construct
QSALTQPASVSGSPGQSITISCAGTSSDVGADNYVSWYQQHPGKAPKLMIYDLSKRPSGVSNRFSGSKSGNTASLTISGLQADDEADYYCSSYTSSSTYVFGTGTKVTVL

SEQ ID NO: 699 - protein, 9 aa, synthetic construct
SSDVGADNY

SEQ ID NO: 700 - skipped

SEQ ID NO: 701 - protein, 10 aa, synthetic construct
SSYTSSSTYV

SEQ ID NO: 702 - protein, 127 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVSTWVRGAHNSYYYYGMDVWGQGTTVTVSS

SEQ ID NO: 703 - protein, 8 aa, synthetic construct
GGSFSGYY

SEQ ID NO: 704 - protein, 7 aa, synthetic construct
INHSGST

SEQ ID NO: 705 - protein, 21 aa, synthetic construct
ARVSTWVRGAHNSYYYYGMDV

SEQ ID NO: 706 - protein, 109 aa, synthetic construct
QSALTQPASVSGSPGQSITISCTGTSSDVGGYNYVSWYQQHPGKVPKLMIYGVTNRPSGVSNRFSGSKSGNTASLTISGLQADDEADYYCSSFTTGSFVFGTGTKVTVL

SEQ ID NO: 707 - protein, 9 aa, synthetic construct
SSDVGGYNY

SEQ ID NO: 708 - skipped

SEQ ID NO: 709 - protein, 9 aa, synthetic construct
SSFTTGSFV

SEQ ID NO: 710 - protein, 135 aa, synthetic construct
EVHLVESGAEVKKPGSSVKVSCKASGGTFSSYAISWVRQAPGQGLEWMGGIIPIFGTANYAQKFQGRVTITADESTSTAYMELSSLRSEDTAVYYCASPGRERAAAGPVAPSLPGPYYYYYMDVWGKGTTVTVSS

SEQ ID NO: 711 - protein, 8 aa, synthetic construct
GGTFSSYA

SEQ ID NO: 712 - protein, 8 aa, synthetic construct
IIPIFGTA

SEQ ID NO: 713 - protein, 28 aa, synthetic construct
ASPGRERAAAGPVAPSLPGPYYYYYMDV

SEQ ID NO: 714 - protein, 121 aa, synthetic construct
QVQRQESGGGLVQPGGSLRLSCSVSGNIDRFYAMGWYRQAPGKQRELVAQLTNNEITTYGDSVEGQFSISGDFDANTVYLQMDSLKPEDTAAYYCYYRIMSLTHTGPRYSWGQGTQVTVSS

SEQ ID NO: 715 - protein, 8 aa, synthetic construct
GNIDRFYA

SEQ ID NO: 716 - protein, 7 aa, synthetic construct
LTNNEIT

SEQ ID NO: 717 - protein, 15 aa, synthetic construct
YYRIMSLTHTGPRYS

SEQ ID NO: 718 - protein, 113 aa, synthetic construct
EVQLVESGGGLVQPGGSLTLACAASGFTFSSATQTWVRQGPGKGLEWVSDINRGDGNGYYADSVKGRFTISRDNAKNTLYLEMNNLKPEDTAVYYCGKGFGAWGQGTQVTVSS

SEQ ID NO: 719 - protein, 8 aa, synthetic construct
GFTFSSAT

SEQ ID NO: 720 - protein, 8 aa, synthetic construct
INRGDGNG

SEQ ID NO: 721 - protein, 6 aa, synthetic construct
GKGFGA

SEQ ID NO: 722 - protein, 117 aa, synthetic construct
QVKLEESGGGLVQPGGSLRLSCAISGSVFSIDALGWYRQAPGQQRELVAHISRTGSTHYSDFVLGRFTISRDNARNSVYLQMNNLKPEDTAVYFCNADLGPRADPVWGRGTSVTVSS

SEQ ID NO: 723 - protein, 8 aa, synthetic construct
GSVFSIDA

SEQ ID NO: 724 - protein, 7 aa, synthetic construct
ISRTGST

SEQ ID NO: 725 - protein, 11 aa, synthetic construct
NADLGPRADPV

SEQ ID NO: 726 - protein, 128 aa, synthetic construct
QVKLEESGGGLVQAGGSLRLSCAASPRTSGGYTMAWFRQASGKERDFVARTGWISDSTDYADSVKSRFTISRDSAKDTLYLQMNSLKPEDTAVYYCAASRKYYSSNSYYREVPTYDYWGQGTQVTVSS

SEQ ID NO: 727 - protein, 8 aa, synthetic construct
PRTSGGYT

SEQ ID NO: 728 - protein, 8 aa, synthetic construct
TGWISDST

SEQ ID NO: 729 - protein, 21 aa, synthetic construct
AASRKYYSSNSYYREVPTYDY

SEQ ID NO: 730 - protein, 115 aa, synthetic construct
QVQLVESGGGLGQPGGSLRLSCVASGGTFAREPIHWYRQTPGNQRELVASIFLTRSPHYADFVKGRFTVSRDVAANTAYLQMNSLKPEDTAIYYCALEYQLRHYWGQGTQVTVSS

SEQ ID NO: 731 - protein, 8 aa, synthetic construct
GGTFAREP

SEQ ID NO: 732 - protein, 7 aa, synthetic construct
IFLTRSP

SEQ ID NO: 733 - protein, 9 aa, synthetic construct
ALEYQLRHY

SEQ ID NO: 734 - protein, 126 aa, synthetic construct
EVQLVESGGGSVQAGDSLRLSCAASGGTFRSYTMGWFRQTPGKEREFVAALGGSIWPAPYYTDSVKGRFNISGDNAKSTVYLQMNSLKQEDTAVYYCAAGGSDYSLRLSPSDYDYWGQGTQVTVSP

SEQ ID NO: 735 - protein, 8 aa, synthetic construct
GGTFRSYT

SEQ ID NO: 736 - protein, 8 aa, synthetic construct
LGGSIWPA

SEQ ID NO: 737 - protein, 19 aa, synthetic construct
CAAGGSDYSLRLSPSDYDY

SEQ ID NO: 738 - protein, 128 aa, synthetic construct
AVQLVDSGGGRVQPGGSLRLSCAASGNTLELYAIGWFRQGPGKEREGISCITSYDGDTYYTDSVKGRFTIPRDNARNSVYLHMNNLKPEDTGVYYCAAGTTFTTMQSVCIRTGWYSTWGQGTQVTVSS

SEQ ID NO: 739 - protein, 8 aa, synthetic construct
GNTLELYA

SEQ ID NO: 740 - protein, 8 aa, synthetic construct
ITSYDGDT

SEQ ID NO: 741 - protein, 21 aa, synthetic construct
AAGTTFTTMQSVCIRTGWYST

SEQ ID NO: 742 - protein, 121 aa, synthetic construct
DVQLVESGGGLVQPGGSLRLSCTASGSTFSDSWMYWVRQAPGKGLELVSPINSYGGATYYADSVKGRFTISRDNAKNTLYLQMNSLKPEDTALYYCATHDSDQGIYQFNYWGQGTQVTVSS

SEQ ID NO: 743 - protein, 8 aa, synthetic construct
GSTFSDSW

SEQ ID NO: 744 - protein, 8 aa, synthetic construct
INSYGGAT

SEQ ID NO: 745 - protein, 14 aa, synthetic construct
ATHDSDQGIYQFNY

SEQ ID NO: 746 - protein, 125 aa, synthetic construct
AVQLVDSGGGLVQPGGSLRLSCAASGFTFDDYAMSWVRQAPGKGLEWVSAISWNDGRTYYAESMKGRFTISRDNAKNTLYLQMNSLKSEDTAVYYCAKAYEDLETIPNHQGFGSWGQGIQVTVSS

SEQ ID NO: 747 - protein, 8 aa, synthetic construct
GFTFDDYA

SEQ ID NO: 748 - protein, 8 aa, synthetic construct
ISWNDGRT

SEQ ID NO: 749 - protein, 18 aa, synthetic construct
AKAYEDLETIPNHQGFGS

SEQ ID NO: 750 - protein, 126 aa, synthetic construct
QVKLEESGGGLVQPGGSLRLSCAASGFTFSSSAMSWVRQAPGKGLEWVSSIYNYGSNTYYADSVKGRLTISTDNAKNTLYLQMNSLKSGDTAVYYCAKEAPLYYSDYDSGTSFGSWGQGTQVTVSS

SEQ ID NO: 751 - protein, 8 aa, synthetic construct
GFTFSSSA

SEQ ID NO: 752 - protein, 8 aa, synthetic construct
IYNYGSNT

SEQ ID NO: 753 - protein, 19 aa, synthetic construct
AKEAPLYYSDYDSGTSFGS

SEQ ID NO: 754 - protein, 125 aa, synthetic construct
QVKLEESGGGLVQPGGSLRLSCAASGFTFSTSTMSWVRQAPGKGLEWVSSIYSYISNTYYADSVKGRFTISTDNAKNTLYLRMNSLKSEDTAVYYCAKTELSSFGLGLNLGYDYWGQGTQVTVSS

SEQ ID NO: 755 - protein, 8 aa, synthetic construct
GFTFSTST

SEQ ID NO: 756 - protein, 8 aa, synthetic construct
IYSYISNT

SEQ ID NO: 757 - protein, 18 aa, synthetic construct
AKTELSSFGLGLNLGYDY

SEQ ID NO: 758 - protein, 123 aa, synthetic construct
AVQLVDSGGGLVKPGGSLRLSCVASGSIFGMNAMGWYRQVLGQERELVAAITKDGETMYAESVKGRFTISGDKAKHTVSLQMNNLNVEDTAVYYCKAAVVNLFGGSTYEYDYWGQGTQVTVSS

SEQ ID NO: 759 - protein, 8 aa, synthetic construct
GSIFGMNA

SEQ ID NO: 760 - protein, 7 aa, synthetic construct
ITKDGET

SEQ ID NO: 761 - protein, 17 aa, synthetic construct
KAAVVNLFGGSTYEYDY

SEQ ID NO: 762 - protein, 118 aa, synthetic construct
QVQLVESGGGSVQPGGSLRLSCAVDAFTFESFHIGWYREAPGKEREGVSCMANRGGSTWTADSVQGRFTISRDNTKRTVYLQMDSLRLEDTATYYCATCGSDSPLGYSGKGTLVTVSS

SEQ ID NO: 763 - protein, 8 aa, synthetic construct
AFTFESFH

SEQ ID NO: 764 - protein, 8 aa, synthetic construct
MANRGGST

SEQ ID NO: 765 - protein, 11 aa, synthetic construct
ATCGSDSPLGY

SEQ ID NO: 766 - protein, 115 aa, synthetic construct
AVQLVDSGGGLAQPGGSLRLSCAASGFTFTSYWMHWVRQAPGKGLEWVSVITPDGASTRYAESVKGRFTISRDNAKNTLYLQMNSLKPEDTAVYYCISPPAMESWGKGTLVTVSS

SEQ ID NO: 767 - protein, 8 aa, synthetic construct
GFTFTSYW

SEQ ID NO: 768 - protein, 8 aa, synthetic construct
ITPDGAST

SEQ ID NO: 769 - protein, 8 aa, synthetic construct
ISPPAMES

SEQ ID NO: 770 - protein, 5 aa, synthetic construct
AYDMD

SEQ ID NO: 771 - protein, 17 aa, synthetic construct
KISHDGSYSWYADSVKG

SEQ ID NO: 772 - protein, 10 aa, synthetic construct
GEGGSERADY

SEQ ID NO: 773 - DNA, 357 bp, synthetic construct
  source 1..357: mol_type = other DNA
caggtgcagctggtgcagtctgggggaggcttggtacagcctggagggtccctgagactctcctgtgcagcctcttatttcgatttcgctgcttatgatatggactgggtccgccaggctccaggacaacgccttgagtgggtggcaaaaatttcacatgatggaagttatagttggtatgcagactctgtgaagggccgattcaccatctccagagacaattccaagaacacgctgtatctgcaaatgaacaccctgagagccgaggacacggccgtatattactgtgtgagaggggaaggtggcagtgaacgcgctgattactggggccggggaaccctggtcaccgtctcttca

SEQ ID NO: 774 - protein, 119 aa, synthetic construct
QVQLVQSGGGLVQPGGSLRLSCAASYFDFAAYDMDWVRQAPGQRLEWVAKISHDGSYSWYADSVKGRFTISRDNSKNTLYLQMNTLRAEDTAVYYCVRGEGGSERADYWGRGTLVTVSS

SEQ ID NO: 775 - protein, 5 aa, synthetic construct
DSSMD

SEQ ID NO: 776 - protein, 16 aa, synthetic construct
SISYFGSTYYNPSLKS

SEQ ID NO: 777 - protein, 11 aa, synthetic construct
SALYGDAAFDV

SEQ ID NO: 778 - DNA, 354 bp, synthetic construct
  source 1..354: mol_type = other DNA
caggtgcagctggtgcagtctgggggaggcttggtacagcctggagggtccctgagactctcctgtgcagcctctgctttcgatttcgatgattcttctatggactgggtccgccaggctccagggaaggccctggagtggattgggagtatcagttattttgggagtacctactacaacccgtccctcaagagtcgagtcaccatctccagagacaattccaagaacacgctgtatctgcaaatgaacaccctgagagccgaggacacagccacatattactgtacaagatcggccttatacggagacgcagccttcgatgtctgggcccaagggacaacggtcactgtctct

SEQ ID NO: 779 - protein, 118 aa, synthetic construct
QVQLVQSGGGLVQPGGSLRLSCAASAFDFDDSSMDWVRQAPGKALEWIGSISYFGSTYYNPSLKSRVTISRDNSKNTLYLQMNTLRAEDTATYYCTRSALYGDAAFDVWAQGTTVTVS

SEQ ID NO: 780 - protein, 5 aa, synthetic construct
YYEMS

SEQ ID NO: 781 - protein, 17 aa, synthetic construct
LMSGNGGSTYYADSVKG

SEQ ID NO: 782 - protein, 7 aa, synthetic construct
HSSTWFS

SEQ ID NO: 783 - DNA, 348 bp, synthetic construct
  source 1..348: mol_type = other DNA
caggtgcagctggtgcagtctgggggaggcttggtacagcctggagggtccctgagactctcctgtgcagcctcttatttcgatttctcttattatgaaatgagctgggtccgccaggctccagggaaggggctggagtgggtctcacttatgagtggtaatggtggtagcacatactacgcagactcagtgaagggccgattcaccatctccagagacaattctaagaacacgctgtatctgcaaatgaataccctgagagccgaggacacagccacgtattactgtgtggcccacagcagcacctggttttcttggggccaaggaaccctggtcaccgtctcctca

SEQ ID NO: 784 - protein, 116 aa, synthetic construct
QVQLVQSGGGLVQPGGSLRLSCAASYFDFSYYEMSWVRQAPGKGLEWVSLMSGNGGSTYYADSVKGRFTISRDNSKNTLYLQMNTLRAEDTATYYCVAHSSTWFSWGQGTLVTVSS

SEQ ID NO: 785 - protein, 5 aa, synthetic construct
DDSMD

SEQ ID NO: 786 - protein, 16 aa, synthetic construct
SVLHDGATYYNPSLKS

SEQ ID NO: 787 - DNA, 357 bp, synthetic construct
  source 1..357: mol_type = other DNA
caggtgcagctggtgcagtctgggggaggcttggtacagcctggagggtccctgagactctcctgtgcagcctctgatttcgatttcgatgatgattctatggactgggtccgccaggctccagggaaggggctagagtggattgggagtgtcttacatgacggagccacctattacaacccgtccctcaagagtcgagtcaccatctccagagacaattccaagaacacgctgtatctgcaaatgaacaccctgagagccgaggacacagccacatattactgtacaagatcggccttatacggggacgcggccttcgatgtctgggcccaagggacaatggtcaccgtctcttca

SEQ ID NO: 788 - protein, 119 aa, synthetic construct
QVQLVQSGGGLVQPGGSLRLSCAASDFDFDDDSMDWVRQAPGKGLEWIGSVLHDGATYYNPSLKSRVTISRDNSKNTLYLQMNTLRAEDTATYYCTRSALYGDAAFDVWAQGTMVTVSS

SEQ ID NO: 789 - protein, 5 aa, synthetic construct
DYEMS

SEQ ID NO: 790 - protein, 16 aa, synthetic construct
LISSDGTTYYADSVKG

SEQ ID NO: 791 - protein, 9 aa, synthetic construct
TTDSWELDL

SEQ ID NO: 792 - DNA, 351 bp, synthetic construct
  source 1..351: mol_type = other DNA
caggtgcagctggtgcagtctgggggaggcttggtacagcctggagggtccctgagactctcctgtgcagcctctgatttctatttcgatgattatgaaatgagctgggtccgccaggctccaggacaacgccttgagtgggtctcacttatctctagcgacggtaccacatactacgcagactctgtgaagggccgattcaccatctccagagacaattccaagaacacgctgtatctgcaaatgaacaccctgagagccgaggacacggctttgtattactgtgtgcgaaccacggactcctgggagctcgatctctggggccgtggcaccctggtcaccgtctcctca

SEQ ID NO: 793 - protein, 117 aa, synthetic construct
QVQLVQSGGGLVQPGGSLRLSCAASDFYFDDYEMSWVRQAPGQRLEWVSLISSDGTTYYADSVKGRFTISRDNSKNTLYLQMNTLRAEDTALYYCVRTTDSWELDLWGRGTLVTVSS

SEQ ID NO: 794 - protein, 5 aa, synthetic construct
SYEMS

SEQ ID NO: 795 - protein, 17 aa, synthetic construct
YIGPIGTDIGYADSVKG

SEQ ID NO: 796 - protein, 12 aa, synthetic construct
DPYYDSTGLTSY

SEQ ID NO: 797 - DNA, 363 bp, synthetic construct
  source 1..363: mol_type = other DNA
caggtgcagctggtgcagtctgggggaggcttggtacagcctggagggtccctgagactctcctgtgcagcctctgctttcgatttctattcttatgaaatgagctgggtccgccaggctccagggaaggggctagagtgggtttcatacattggtcccattggtactgacataggctacgcagactccgtgaagggccgattcaccatctccagagacaattccaagaacacgctgtatctgcaaatgaacaccctgagagccgaggacacagccatgtattactgtgcgaaagacccttactatgatagtactgggttaacgagctactggggccagggaaccctggtcaccgtctcctca

SEQ ID NO: 798 - protein, 121 aa, synthetic construct
QVQLVQSGGGLVQPGGSLRLSCAASAFDFYSYEMSWVRQAPGKGLEWVSYIGPIGTDIGYADSVKGRFTISRDNSKNTLYLQMNTLRAEDTAMYYCAKDPYYDSTGLTSYWGQGTLVTVSS

SEQ ID NO: 799 - protein, 16 aa, synthetic construct
SVSYSGQTFYNPSLKS

SEQ ID NO: 800 - DNA, 357 bp, synthetic construct
  source 1..357: mol_type = other DNA
caggtgcagctggtgcagtctgggggaggcttggtacagcctggagggtccctgagactctcctgtgcagcctctgatttcgatttcgatgatgattctatggactgggtccgccaggctccagggaaggccctggagtggattgggagtgtctcttatagtggacaaaccttctacaacccgtccctcaagagtcgagtcaccatctccagagacaattccaagaacacgctgtatctgcaaatgaacaccctgagagccgaggacacagccatgtattactgtacaagatcggccttatacggagacgcggccttcgatgtctgggcccaagggacaatggtcaccgtctcctca

SEQ ID NO: 801 - protein, 119 aa, synthetic construct
QVQLVQSGGGLVQPGGSLRLSCAASDFDFDDDSMDWVRQAPGKALEWIGSVSYSGQTFYNPSLKSRVTISRDNSKNTLYLQMNTLRAEDTAMYYCTRSALYGDAAFDVWAQGTMVTVSS

SEQ ID NO: 802 - protein, 16 aa, synthetic construct
SIFYSGTTYYNPSLKS

SEQ ID NO: 803 - DNA, 357 bp, synthetic construct
  source 1..357: mol_type = other DNA
caggtgcagctggtgcagtctgggggaggcttggtacagcctggagggtccctgagactctcctgtgcagcctctgatttcgatttcgctgatgattctatggactgggtccgccaggctccagggaaggccctggagtggattgggagtatcttttatagtgggaccacctactacaacccgtccctcaagagtcgagtcaccatctccagagacaattccaagaacacgctgtatctgcaaatgaacaccctgagagccgaggacacagccgtatattactgtaccagatcggccttatacggagacgcggccttcgatgtctgggcccaagggacaatggtcaccgtctcctca

SEQ ID NO: 804 - protein, 119 aa, synthetic construct
QVQLVQSGGGLVQPGGSLRLSCAASDFDFADDSMDWVRQAPGKALEWIGSIFYSGTTYYNPSLKSRVTISRDNSKNTLYLQMNTLRAEDTAVYYCTRSALYGDAAFDVWAQGTMVTVSS

SEQ ID NO: 805 - protein, 17 aa, synthetic construct
VVSKDGNELYYADSVKG

SEQ ID NO: 806 - protein, 12 aa, synthetic construct
DPYDFWSGYYSN

SEQ ID NO: 807 - DNA, 363 bp, synthetic construct
  source 1..363: mol_type = other DNA
caggtgcagctggtgcagtctgggggaggcttggtacagcctggagggtccctgagactctcctgtgcagcctcttatttctctttcgctgattatgaaatgagctgggtccgccaggctccaggacaacggcttgagtgggtggcagttgtgtcaaaggatggaaatgaactatattacgcagactcggtgaagggccgattcaccatctccagagacaattccaagaacacgctgtatctgcaaatgaacaccctgagagccgaggacacagctgtgtattactgtgtgaaagatccttacgatttttggagtggttattattcgaactggggccagggaaccctggtcaccgtctcctca

SEQ ID NO: 808 - protein, 121 aa, synthetic construct
QVQLVQSGGGLVQPGGSLRLSCAASYFSFADYEMSWVRQAPGQRLEWVAVVSKDGNELYYADSVKGRFTISRDNSKNTLYLQMNTLRAEDTAVYYCVKDPYDFWSGYYSNWGQGTLVTVSS

SEQ ID NO: 809 - protein, 11 aa, synthetic construct
QASQDINNFLN

SEQ ID NO: 810 - protein, 9 aa, synthetic construct
QQYGNLPFT

SEQ ID NO: 811 - protein, 5 aa, synthetic construct
SYYWS

SEQ ID NO: 812 - protein, 16 aa, synthetic construct
YIYYSGSTNYNPSLKS

SEQ ID NO: 813 - protein, 14 aa, synthetic construct
LVYCGGDCYSGFDY

SEQ ID NO: 814 - DNA, 324 bp, synthetic construct
  source 1..324: mol_type = other DNA
gacatccagctgacccagagccccagcagcctgagcgccagcgtgggcgacagggtgagcttcacctgccaggccagccaggacatcaacaacttcctgaactggtaccagcagaagcccggcaaggcccccaagctgctgatctacgacgccagcaacctggagaccggcgtgcccagcaggttcagcggcagcggcagcggcaccgacttcaccttcaccatcagcagcctgcagcccgaggacatcgccacctactactgccagcagtacggcaacctgcccttcaccttcggcggcggcaccaaggtggagatcaagagg

SEQ ID NO: 815 - protein, 107 aa, synthetic construct
DIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIK

SEQ ID NO: 816 - DNA, 366 bp, synthetic construct
  source 1..366: mol_type = other DNA
caggtgcagctgcaggagagcggccccggcctggtgaagcccagcgagaccctgagcctgacctgcaccgtgagcggcggcagcatcagcagctactactggagctggatcaggcagccccccggcaagggcctggagtggatcggctacatctactacagcggcagcaccaactacaaccccagcctgaagagcagggtgaccatcagcgtggacaccagcaagaaccagttcagcctgaagctgagcagcgtgaccgccgccgacaccgccgtgtactactgcgtgagcctggtgtactgcggcggcgactgctacagcggcttcgactactggggccagggcaccctggtgaccgtgagcagc

SEQ ID NO: 817 - protein, 122 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSS

SEQ ID NO: 818 - DNA, 735 bp, synthetic construct
  source 1..735: mol_type = other DNA
gacatccagctgacccagagccccagcagcctgagcgccagcgtgggcgacagggtgagcttcacctgccaggccagccaggacatcaacaacttcctgaactggtaccagcagaagcccggcaaggcccccaagctgctgatctacgacgccagcaacctggagaccggcgtgcccagcaggttcagcggcagcggcagcggcaccgacttcaccttcaccatcagcagcctgcagcccgaggacatcgccacctactactgccagcagtacggcaacctgcccttcaccttcggcggcggcaccaaggtggagatcaagaggggcggcggcggcagcggcggcggcggcagcggcggcggcggcagccaggtgcagctgcaggagagcggccccggcctggtgaagcccagcgagaccctgagcctgacctgcaccgtgagcggcggcagcatcagcagctactactggagctggatcaggcagccccccggcaagggcctggagtggatcggctacatctactacagcggcagcaccaactacaaccccagcctgaagagcagggtgaccatcagcgtggacaccagcaagaaccagttcagcctgaagctgagcagcgtgaccgccgccgacaccgccgtgtactactgcgtgagcctggtgtactgcggcggcgactgctacagcggcttcgactactggggccagggcaccctggtgaccgtgagcagc

SEQ ID NO: 819 - protein, 245 aa, synthetic construct
DIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRGGGGSGGGGSGGGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSS

SEQ ID NO: 820 - protein, 17 aa, synthetic construct
KSSQSLLYSDNQKNYLA

SEQ ID NO: 821 - protein, 7 aa, synthetic construct
WASTRES

SEQ ID NO: 822 - protein, 9 aa, synthetic construct
QQYYTYPYT

SEQ ID NO: 823 - protein, 5 aa, synthetic construct
GYHMH

SEQ ID NO: 824 - protein, 17 aa, synthetic construct
RINPYNGAASHNQKFKD

SEQ ID NO: 825 - protein, 13 aa, synthetic construct
GWDYDGGYYAMDY

SEQ ID NO: 826 - DNA, 339 bp, synthetic construct
  source 1..339: mol_type = other DNA
gatatccagatgacccagtctcctagtagcctgtctgcctccgtcggcgatcgggtgaccattacttgcaaatcctcacagagcctcctctactccgataatcagaagaactacctcgcctggtatcaacagaaaccagggaaggcacctaagctgctgatctactgggctagtacccgcgaatccggcgtccctagcaggttctctggcagcgggagcgggacagatttcaccctcactatctcctccctgcagcctgaagacttcgcaacttactactgtcagcagtattatacttacccatacactttcggacagggaacaaaactggaaattaaa

SEQ ID NO: 827 - protein, 113 aa, synthetic construct
DIQMTQSPSSLSASVGDRVTITCKSSQSLLYSDNQKNYLAWYQQKPGKAPKLLIYWASTRESGVPSRFSGSGSGTDFTLTISSLQPEDFATYYCQQYYTYPYTFGQGTKLEIK

SEQ ID NO: 828 - DNA, 366 bp, synthetic construct
  source 1..366: mol_type = other DNA
gaagtccaactggtgcagagcggtgcagaggtgaagaagcctggagcatcagtgaaggtgtcttgcaaagccagtggctacacattcactggatatcatatgcattgggttaggcaggcacccggccagcggctggagtggatgggaagaatcaacccttataatggcgctgcctctcacaatcaaaagtttaaggatcgggtcactatcactcgggacacttccgcaagcaccgcctatatggagctgagcagcctgcggagtgaagacacagcagtctactactgtgctcgcggatgggactatgacggcggttattatgccatggattactggggacagggcacactggtcaccgtgagcagc

SEQ ID NO: 829 - protein, 122 aa, synthetic construct
EVQLVQSGAEVKKPGASVKVSCKASGYTFTGYHMHWVRQAPGQRLEWMGRINPYNGAASHNQKFKDRVTITRDTSASTAYMELSSLRSEDTAVYYCARGWDYDGGYYAMDYWGQGTLVTVSS

SEQ ID NO: 830 - DNA, 750 bp, synthetic construct
  source 1..750: mol_type = other DNA
gatatccagatgacccagtctcctagtagcctgtctgcctccgtcggcgatcgggtgaccattacttgcaaatcctcacagagcctcctctactccgataatcagaagaactacctcgcctggtatcaacagaaaccagggaaggcacctaagctgctgatctactgggctagtacccgcgaatccggcgtccctagcaggttctctggcagcgggagcgggacagatttcaccctcactatctcctccctgcagcctgaagacttcgcaacttactactgtcagcagtattatacttacccatacactttcggacagggaacaaaactggaaattaaaggtggaggtggatctggtggcggtggcagtggcggaggcgggtctgaagtccaactggtgcagagcggtgcagaggtgaagaagcctggagcatcagtgaaggtgtcttgcaaagccagtggctacacattcactggatatcatatgcattgggttaggcaggcacccggccagcggctggagtggatgggaagaatcaacccttataatggcgctgcctctcacaatcaaaagtttaaggatcgggtcactatcactcgggacacttccgcaagcaccgcctatatggagctgagcagcctgcggagtgaagacacagcagtctactactgtgctcgcggatgggactatgacggcggttattatgccatggattactggggacagggcacactggtcaccgtgagcagc

SEQ ID NO: 831 - protein, 250 aa, synthetic construct
DIQMTQSPSSLSASVGDRVTITCKSSQSLLYSDNQKNYLAWYQQKPGKAPKLLIYWASTRESGVPSRFSGSGSGTDFTLTISSLQPEDFATYYCQQYYTYPYTFGQGTKLEIKGGGGSGGGGSGGGGSEVQLVQSGAEVKKPGASVKVSCKASGYTFTGYHMHWVRQAPGQRLEWMGRINPYNGAASHNQKFKDRVTITRDTSASTAYMELSSLRSEDTAVYYCARGWDYDGGYYAMDYWGQGTLVTVSS

SEQ ID NO: 832 - DNA, 339 bp, synthetic construct
  source 1..339: mol_type = other DNA
gacatcgtgatgagccagagccccagcagcctggccgtgagcgtgggcgagaaggtgaccatgagctgcaagagcagccagagcctgctgtacagcgacaaccagaagaactacctggcctggtaccagcagaagcccggccagagccccaagctgctgatctactgggccagcaccagggagagcggcgtgcccgacaggttcaccggcagcggcagcggcaccgacttcaccctgaccatcagcagcgtgaaggccgaggacctggccgtgtactactgccagcagtactacacctacccctacaccttcggcggcggcaccaagctggagatcaag

SEQ ID NO: 833 - protein, 113 aa, synthetic construct
DIVMSQSPSSLAVSVGEKVTMSCKSSQSLLYSDNQKNYLAWYQQKPGQSPKLLIYWASTRESGVPDRFTGSGSGTDFTLTISSVKAEDLAVYYCQQYYTYPYTFGGGTKLEIK

SEQ ID NO: 834 - DNA, 366 bp, synthetic construct
  source 1..366: mol_type = other DNA
gaggtgcagctgcagcagagcggccccgagctggtgaagcccggcgccagcgtgaagatcagctgcaaggccagcggctacagcttcaccggctaccacatgcactgggtgaagcagagccacgtgaagagcctggagtggatcggcaggatcaacccctacaacggcgccgccagccacaaccagaagttcaaggacaaggccaccctgaccgtggacaagagcagcagcaccgcctacatggagctgcacagcctgaccagcgaggacagcgccgtgtactactgcgccaggggctgggactacgacggcggctactacgccatggactactggggccagggcaccagcgtgaccgtgagcagc

SEQ ID NO: 835 - protein, 122 aa, synthetic construct
EVQLQQSGPELVKPGASVKISCKASGYSFTGYHMHWVKQSHVKSLEWIGRINPYNGAASHNQKFKDKATLTVDKSSSTAYMELHSLTSEDSAVYYCARGWDYDGGYYAMDYWGQGTSVTVSS

SEQ ID NO: 836 - DNA, 750 bp, synthetic construct
  source 1..750: mol_type = other DNA
gacatcgtgatgagccagagccccagcagcctggccgtgagcgtgggcgagaaggtgaccatgagctgcaagagcagccagagcctgctgtacagcgacaaccagaagaactacctggcctggtaccagcagaagcccggccagagccccaagctgctgatctactgggccagcaccagggagagcggcgtgcccgacaggttcaccggcagcggcagcggcaccgacttcaccctgaccatcagcagcgtgaaggccgaggacctggccgtgtactactgccagcagtactacacctacccctacaccttcggcggcggcaccaagctggagatcaagggcggcggcggcagcggcggcggcggcagcggcggcggcggcagcgaggtgcagctgcagcagagcggccccgagctggtgaagcccggcgccagcgtgaagatcagctgcaaggccagcggctacagcttcaccggctaccacatgcactgggtgaagcagagccacgtgaagagcctggagtggatcggcaggatcaacccctacaacggcgccgccagccacaaccagaagttcaaggacaaggccaccctgaccgtggacaagagcagcagcaccgcctacatggagctgcacagcctgaccagcgaggacagcgccgtgtactactgcgccaggggctgggactacgacggcggctactacgccatggactactggggccagggcaccagcgtgaccgtgagcagc

SEQ ID NO: 837 - protein, 250 aa, synthetic construct
DIVMSQSPSSLAVSVGEKVTMSCKSSQSLLYSDNQKNYLAWYQQKPGQSPKLLIYWASTRESGVPDRFTGSGSGTDFTLTISSVKAEDLAVYYCQQYYTYPYTFGGGTKLEIKGGGGSGGGGSGGGGSEVQLQQSGPELVKPGASVKISCKASGYSFTGYHMHWVKQSHVKSLEWIGRINPYNGAASHNQKFKDKATLTVDKSSSTAYMELHSLTSEDSAVYYCARGWDYDGGYYAMDYWGQGTSVTVSS

SEQ ID NO: 838 - protein, 10 aa, synthetic construct
SASSSVSYMY

SEQ ID NO: 839 - protein, 7 aa, synthetic construct
DTSNLAS

SEQ ID NO: 840 - protein, 9 aa, synthetic construct
QQWSSYPLT

SEQ ID NO: 841 - protein, 5 aa, synthetic construct
SYWIE

SEQ ID NO: 842 - protein, 17 aa, synthetic construct
EIFPGSGSIKYNEKFKG

SEQ ID NO: 843 - protein, 12 aa, synthetic construct
GGTYNDYSLFDY

SEQ ID NO: 844 - DNA, 318 bp, synthetic construct
  source 1..318: mol_type = other DNA
cagatcgtgctgacccagagccccgccatcatgagcgccagccccggcgagaaggtgaccatgacctgcagcgccagcagcagcgtgagctacatgtactggtaccagcagaagcccggcagcagccccaggctgctgatcttcgacaccagcaacctggccagcggcgtgcccgtgaggttcagcggcagcggcagcggcaccagctacagcctgaccatcagcaggatggaggccgaggacgccgccacctactactgccagcagtggagcagctaccccctgaccttcggcgccggcaccaagctggagctgaag

SEQ ID NO: 845 - protein, 106 aa, synthetic construct
QIVLTQSPAIMSASPGEKVTMTCSASSSVSYMYWYQQKPGSSPRLLIFDTSNLASGVPVRFSGSGSGTSYSLTISRMEAEDAATYYCQQWSSYPLTFGAGTKLELK

SEQ ID NO: 846 - DNA, 363 bp, synthetic construct
  source 1..363: mol_type = other DNA
caggtgcagctgcagcagagcggcgccgagctgatgaagcccggcgccagcgtgaagatcagctgcaaggccaccggctacaccttcagcagctactggatcgagtgggtgaagcagaggcccggccacggcctggagtggatcggcgagatcttccccggcagcggcagcatcaagtacaacgagaagttcaagggcaaggccaccttcaccgccgacaccagcagcaacaccgcctacatgcagctgagcagcctgaccagcgaggacagcgccgtgcactactgcgccaggggcggcacctacaacgactacagcctgttcgactactggggccagggcaccaccctgaccgtgagcagc

SEQ ID NO: 847 - protein, 121 aa, synthetic construct
QVQLQQSGAELMKPGASVKISCKATGYTFSSYWIEWVKQRPGHGLEWIGEIFPGSGSIKYNEKFKGKATFTADTSSNTAYMQLSSLTSEDSAVHYCARGGTYNDYSLFDYWGQGTTLTVSS

SEQ ID NO: 848 - DNA, 726 bp, synthetic construct
  source 1..726: mol_type = other DNA
cagatcgtgctgacccagagccccgccatcatgagcgccagccccggcgagaaggtgaccatgacctgcagcgccagcagcagcgtgagctacatgtactggtaccagcagaagcccggcagcagccccaggctgctgatcttcgacaccagcaacctggccagcggcgtgcccgtgaggttcagcggcagcggcagcggcaccagctacagcctgaccatcagcaggatggaggccgaggacgccgccacctactactgccagcagtggagcagctaccccctgaccttcggcgccggcaccaagctggagctgaagggcggcggcggcagcggcggcggcggcagcggcggcggcggcagccaggtgcagctgcagcagagcggcgccgagctgatgaagcccggcgccagcgtgaagatcagctgcaaggccaccggctacaccttcagcagctactggatcgagtgggtgaagcagaggcccggccacggcctggagtggatcggcgagatcttccccggcagcggcagcatcaagtacaacgagaagttcaagggcaaggccaccttcaccgccgacaccagcagcaacaccgcctacatgcagctgagcagcctgaccagcgaggacagcgccgtgcactactgcgccaggggcggcacctacaacgactacagcctgttcgactactggggccagggcaccaccctgaccgtgagcagc

SEQ ID NO: 849 - protein, 242 aa, synthetic construct
QIVLTQSPAIMSASPGEKVTMTCSASSSVSYMYWYQQKPGSSPRLLIFDTSNLASGVPVRFSGSGSGTSYSLTISRMEAEDAATYYCQQWSSYPLTFGAGTKLELKGGGGSGGGGSGGGGSQVQLQQSGAELMKPGASVKISCKATGYTFSSYWIEWVKQRPGHGLEWIGEIFPGSGSIKYNEKFKGKATFTADTSSNTAYMQLSSLTSEDSAVHYCARGGTYNDYSLFDYWGQGTTLTVSS

SEQ ID NO: 850 - protein, 10 aa, synthetic construct
SASSSVSYIY

SEQ ID NO: 851 - protein, 9 aa, synthetic construct
QQWSSFPPT

SEQ ID NO: 852 - protein, 5 aa, synthetic construct
RYWMH

SEQ ID NO: 853 - protein, 17 aa, synthetic construct
YIYPGSGTSNYDEKFKS

SEQ ID NO: 854 - protein, 8 aa, synthetic construct
EARYTMDY

SEQ ID NO: 855 - DNA, 318 bp, synthetic construct
  source 1..318: mol_type = other DNA
cagatcgtgctgacccagagccccgccatcatgagcgccagccccggcgagaaggtgaccatgacctgcagcgccagcagcagcgtgagctacatctactggtaccagcagaagcccggcagcagccccggcctgctgatctacgacaccagcaacctggccagcggcgtgcccgtgaggttcagcggcagcggcagcggcaccagctacagcctgaccatcagcaggatggaggccgaggacgccgccacctactactgccagcagtggagcagcttcccccccaccttcggcgccggcaccaagctggagctgaag

SEQ ID NO: 856 - protein, 106 aa, synthetic construct
QIVLTQSPAIMSASPGEKVTMTCSASSSVSYIYWYQQKPGSSPGLLIYDTSNLASGVPVRFSGSGSGTSYSLTISRMEAEDAATYYCQQWSSFPPTFGAGTKLELK

SEQ ID NO: 857 - DNA, 351 bp, synthetic construct
  source 1..351: mol_type = other DNA
caggtgcagctgcagcagcccggcagcgacctggtgaggcccggcgccagcgtgaagctgagctgcaaggccagcggctacaccttcaccaggtactggatgcactgggtgaagcagaggcccggccacggcctggagtggatcggctacatctaccccggcagcggcaccagcaactacgacgagaagttcaagagcaaggccaccctgaccgtggacaccagcagcagcaccgcctacatgcagctgagcagcctgaccagcgaggacagcgccgtgtactactgcaccagggaggccaggtacaccatggactactggggccagggcaccagcgtgaccgtgagcagc

SEQ ID NO: 858 - protein, 117 aa, synthetic construct
QVQLQQPGSDLVRPGASVKLSCKASGYTFTRYWMHWVKQRPGHGLEWIGYIYPGSGTSNYDEKFKSKATLTVDTSSSTAYMQLSSLTSEDSAVYYCTREARYTMDYWGQGTSVTVSS

SEQ ID NO: 859 - DNA, 714 bp, synthetic construct
  source 1..714: mol_type = other DNA
cagatcgtgctgacccagagccccgccatcatgagcgccagccccggcgagaaggtgaccatgacctgcagcgccagcagcagcgtgagctacatctactggtaccagcagaagcccggcagcagccccggcctgctgatctacgacaccagcaacctggccagcggcgtgcccgtgaggttcagcggcagcggcagcggcaccagctacagcctgaccatcagcaggatggaggccgaggacgccgccacctactactgccagcagtggagcagcttcccccccaccttcggcgccggcaccaagctggagctgaagggcggcggcggcagcggcggcggcggcagcggcggcggcggcagccaggtgcagctgcagcagcccggcagcgacctggtgaggcccggcgccagcgtgaagctgagctgcaaggccagcggctacaccttcaccaggtactggatgcactgggtgaagcagaggcccggccacggcctggagtggatcggctacatctaccccggcagcggcaccagcaactacgacgagaagttcaagagcaaggccaccctgaccgtggacaccagcagcagcaccgcctacatgcagctgagcagcctgaccagcgaggacagcgccgtgtactactgcaccagggaggccaggtacaccatggactactggggccagggcaccagcgtgaccgtgagcagc

SEQ ID NO: 860 - protein, 238 aa, synthetic construct
QIVLTQSPAIMSASPGEKVTMTCSASSSVSYIYWYQQKPGSSPGLLIYDTSNLASGVPVRFSGSGSGTSYSLTISRMEAEDAATYYCQQWSSFPPTFGAGTKLELKGGGGSGGGGSGGGGSQVQLQQPGSDLVRPGASVKLSCKASGYTFTRYWMHWVKQRPGHGLEWIGYIYPGSGTSNYDEKFKSKATLTVDTSSSTAYMQLSSLTSEDSAVYYCTREARYTMDYWGQGTSVTVSS

SEQ ID NO: 861 - protein, 11 aa, synthetic construct
QASQDVRTWLA

SEQ ID NO: 862 - protein, 7 aa, synthetic construct
SASSLQT

SEQ ID NO: 863 - protein, 9 aa, synthetic construct
QQSNYLPFT

SEQ ID NO: 864 - protein, 5 aa, synthetic construct
SYDMS

SEQ ID NO: 865 - protein, 17 aa, synthetic construct
AINPSGDFTSYNEKFQG

SEQ ID NO: 866 - protein, 9 aa, synthetic construct
GVGWSFLDY

SEQ ID NO: 867 - DNA, 327 bp, synthetic construct
  source 1..327: mol_type = other DNA
gacatccagatgacccagagccccagcagcctgagcgccagcgtgggcgacagggtgaccatcacctgccaggccagccaggacgtgaggacctggctggcctggtaccagcagaagcccggcaaggcccccaagctgctgatctacagcgccagcagcctgcagaccggcgtgcccagcaggttcagcggcagcggcagcggcaccgacttcaccctgaccatcagcagcctgcagcccgaggacttcgccacctactactgccagcagagcaactacctgcccttcaccttcggccagggcaccaaggtggagatcaagagcagc

SEQ ID NO: 868 - protein, 109 aa, synthetic construct
DIQMTQSPSSLSASVGDRVTITCQASQDVRTWLAWYQQKPGKAPKLLIYSASSLQTGVPSRFSGSGSGTDFTLTISSLQPEDFATYYCQQSNYLPFTFGQGTKVEIKSS

SEQ ID NO: 869 - DNA, 354 bp, synthetic construct
  source 1..354: mol_type = other DNA
caggtgcagctggtgcagagcggcgccgaggtgaagaagcccggcgccagcgtgaaggtgagctgcaaggccagcggctacaccttcaccagctacgacatgagctgggtgaggcaggcccccggccagggcctggagtggatgggcgccatcaaccccagcggcgacttcaccagctacaacgagaagttccagggcagggtgaccatgaccagggacaccagcaccagcaccgtgtacatggagctgagcagcctgaggagcgaggacaccgccgtgtactactgcgccaggggcgtgggctggagcttcctggactactggggccagggcaccctggtgaccgtgagcagc

SEQ ID NO: 870 - protein, 118 aa, synthetic construct
QVQLVQSGAEVKKPGASVKVSCKASGYTFTSYDMSWVRQAPGQGLEWMGAINPSGDFTSYNEKFQGRVTMTRDTSTSTVYMELSSLRSEDTAVYYCARGVGWSFLDYWGQGTLVTVSS

SEQ ID NO: 871 - DNA, 726 bp, synthetic construct
  source 1..726: mol_type = other DNA
caggtgcagctggtgcagagcggcgccgaggtgaagaagcccggcgccagcgtgaaggtgagctgcaaggccagcggctacaccttcaccagctacgacatgagctgggtgaggcaggcccccggccagggcctggagtggatgggcgccatcaaccccagcggcgacttcaccagctacaacgagaagttccagggcagggtgaccatgaccagggacaccagcaccagcaccgtgtacatggagctgagcagcctgaggagcgaggacaccgccgtgtactactgcgccaggggcgtgggctggagcttcctggactactggggccagggcaccctggtgaccgtgagcagcggcggcggcggcagcggcggcggcggcagcggcggcggcggcagcgacatccagatgacccagagccccagcagcctgagcgccagcgtgggcgacagggtgaccatcacctgccaggccagccaggacgtgaggacctggctggcctggtaccagcagaagcccggcaaggcccccaagctgctgatctacagcgccagcagcctgcagaccggcgtgcccagcaggttcagcggcagcggcagcggcaccgacttcaccctgaccatcagcagcctgcagcccgaggacttcgccacctactactgccagcagagcaactacctgcccttcaccttcggccagggcaccaaggtggagatcaagagcagc

SEQ ID NO: 872 - protein, 242 aa, synthetic construct
QVQLVQSGAEVKKPGASVKVSCKASGYTFTSYDMSWVRQAPGQGLEWMGAINPSGDFTSYNEKFQGRVTMTRDTSTSTVYMELSSLRSEDTAVYYCARGVGWSFLDYWGQGTLVTVSSGGGGSGGGGSGGGGSDIQMTQSPSSLSASVGDRVTITCQASQDVRTWLAWYQQKPGKAPKLLIYSASSLQTGVPSRFSGSGSGTDFTLTISSLQPEDFATYYCQQSNYLPFTFGQGTKVEIKSS

SEQ ID NO: 873 - protein, 11 aa, synthetic construct
KASEDIHSDLA

SEQ ID NO: 874 - protein, 9 aa, synthetic construct
QQAYYLPLT

SEQ ID NO: 875 - protein, 5 aa, synthetic construct
DYAMD

SEQ ID NO: 876 - protein, 17 aa, synthetic construct
WISGQSGSTSYNEKFQG

SEQ ID NO: 877 - protein, 9 aa, synthetic construct
WLDGPWFDV

SEQ ID NO: 878 - DNA, 327 bp, synthetic construct
  source 1..327: mol_type = other DNA
gacatccagatgacccagagccccagcagcctgagcgccagcgtgggcgacagggtgaccatcacctgcaaggccagcgaggacatccacagcgacctggcctggtaccagcagaagcccggcaaggcccccaagctgctgatctacagcgccagcagcctgcagagcggcgtgcccagcaggttcagcggcagcggcagcggcaccgacttcaccctgaccatcagcagcctgcagcccgaggacttcgccacctactactgccagcaggcctactacctgcccctgaccttcggccagggcaccaaggtggagatcaagagcagc

SEQ ID NO: 879 - protein, 109 aa, synthetic construct
DIQMTQSPSSLSASVGDRVTITCKASEDIHSDLAWYQQKPGKAPKLLIYSASSLQSGVPSRFSGSGSGTDFTLTISSLQPEDFATYYCQQAYYLPLTFGQGTKVEIKSS

SEQ ID NO: 880 - DNA, 354 bp, synthetic construct
  source 1..354: mol_type = other DNA
caggtgcagctggtgcagagcggcgccgaggtgaagaagcccggcgccagcgtgaaggtgagctgcaaggccagcggctacaccttcaccgactacgccatggactgggtgaggcaggcccccggccagggcctggagtggatgggctggatcagcggccagagcggcagcaccagctacaacgagaagttccagggcagggtgaccatgaccagggacaccagcaccagcaccgtgtacatggagctgagcagcctgaggagcgaggacaccgccgtgtactactgcgccaagtggctggacggcccctggttcgacgtgtggggccagggcaccctggtgaccgtgagcagc

SEQ ID NO: 881 - protein, 118 aa, synthetic construct
QVQLVQSGAEVKKPGASVKVSCKASGYTFTDYAMDWVRQAPGQGLEWMGWISGQSGSTSYNEKFQGRVTMTRDTSTSTVYMELSSLRSEDTAVYYCAKWLDGPWFDVWGQGTLVTVSS

SEQ ID NO: 882 - DNA, 726 bp, synthetic construct
  source 1..726: mol_type = other DNA
caggtgcagctggtgcagagcggcgccgaggtgaagaagcccggcgccagcgtgaaggtgagctgcaaggccagcggctacaccttcaccgactacgccatggactgggtgaggcaggcccccggccagggcctggagtggatgggctggatcagcggccagagcggcagcaccagctacaacgagaagttccagggcagggtgaccatgaccagggacaccagcaccagcaccgtgtacatggagctgagcagcctgaggagcgaggacaccgccgtgtactactgcgccaagtggctggacggcccctggttcgacgtgtggggccagggcaccctggtgaccgtgagcagcggcggcggcggcagcggcggcggcggcagcggcggcggcggcagcgacatccagatgacccagagccccagcagcctgagcgccagcgtgggcgacagggtgaccatcacctgcaaggccagcgaggacatccacagcgacctggcctggtaccagcagaagcccggcaaggcccccaagctgctgatctacagcgccagcagcctgcagagcggcgtgcccagcaggttcagcggcagcggcagcggcaccgacttcaccctgaccatcagcagcctgcagcccgaggacttcgccacctactactgccagcaggcctactacctgcccctgaccttcggccagggcaccaaggtggagatcaagagcagc

SEQ ID NO: 883 - protein, 242 aa, synthetic construct
QVQLVQSGAEVKKPGASVKVSCKASGYTFTDYAMDWVRQAPGQGLEWMGWISGQSGSTSYNEKFQGRVTMTRDTSTSTVYMELSSLRSEDTAVYYCAKWLDGPWFDVWGQGTLVTVSSGGGGSGGGGSGGGGSDIQMTQSPSSLSASVGDRVTITCKASEDIHSDLAWYQQKPGKAPKLLIYSASSLQSGVPSRFSGSGSGTDFTLTISSLQPEDFATYYCQQAYYLPLTFGQGTKVEIKSS

SEQ ID NO: 884 - protein, 11 aa, synthetic construct
QASQDVSTDLA

SEQ ID NO: 885 - protein, 7 aa, synthetic construct
HASSLES

SEQ ID NO: 886 - protein, 9 aa, synthetic construct
QQYYNYPLT

SEQ ID NO: 887 - protein, 5 aa, synthetic construct
HYAMD

SEQ ID NO: 888 - protein, 17 aa, synthetic construct
GISPQSGGTSYNEKFQG

SEQ ID NO: 889 - protein, 9 aa, synthetic construct
VHGDYHFDY

SEQ ID NO: 890 - DNA, 327 bp, synthetic construct
  source 1..327: mol_type = other DNA
gacatccagatgacccagagccccagcagcctgagcgccagcgtgggcgacagggtgaccatcacctgccaggccagccaggacgtgagcaccgacctggcctggtaccagcagaagcccggcaaggcccccaagctgctgatctaccacgccagcagcctggagagcggcgtgcccagcaggttcagcggcagcggcagcggcaccgacttcaccctgaccatcagcagcctgcagcccgaggacttcgccacctactactgccagcagtactacaactaccccctgaccttcggccagggcaccaaggtggagatcaagagcagc

SEQ ID NO: 891 - protein, 109 aa, synthetic construct
DIQMTQSPSSLSASVGDRVTITCQASQDVSTDLAWYQQKPGKAPKLLIYHASSLESGVPSRFSGSGSGTDFTLTISSLQPEDFATYYCQQYYNYPLTFGQGTKVEIKSS

SEQ ID NO: 892 - DNA, 354 bp, synthetic construct
  source 1..354: mol_type = other DNA
caggtgcagctggtgcagagcggcgccgaggtgaagaagcccggcgccagcgtgaaggtgagctgcaaggccagcggctacaccttcacccactacgccatggactgggtgaggcaggcccccggccagggcctggagtggatgggcggcatcagcccccagagcggcggcaccagctacaacgagaagttccagggcagggtgaccatgaccagggacaccagcaccagcaccgtgtacatggagctgagcagcctgaggagcgaggacaccgccgtgtactactgcgccagggtgcacggcgactaccacttcgactactggggccagggcaccctggtgaccgtgagcagc

SEQ ID NO: 893 - protein, 118 aa, synthetic construct
QVQLVQSGAEVKKPGASVKVSCKASGYTFTHYAMDWVRQAPGQGLEWMGGISPQSGGTSYNEKFQGRVTMTRDTSTSTVYMELSSLRSEDTAVYYCARVHGDYHFDYWGQGTLVTVSS

SEQ ID NO: 894 - DNA, 726 bp, synthetic construct
  source 1..726: mol_type = other DNA
caggtgcagctggtgcagagcggcgccgaggtgaagaagcccggcgccagcgtgaaggtgagctgcaaggccagcggctacaccttcacccactacgccatggactgggtgaggcaggcccccggccagggcctggagtggatgggcggcatcagcccccagagcggcggcaccagctacaacgagaagttccagggcagggtgaccatgaccagggacaccagcaccagcaccgtgtacatggagctgagcagcctgaggagcgaggacaccgccgtgtactactgcgccagggtgcacggcgactaccacttcgactactggggccagggcaccctggtgaccgtgagcagcggcggcggcggcagcggcggcggcggcagcggcggcggcggcagcgacatccagatgacccagagccccagcagcctgagcgccagcgtgggcgacagggtgaccatcacctgccaggccagccaggacgtgagcaccgacctggcctggtaccagcagaagcccggcaaggcccccaagctgctgatctaccacgccagcagcctggagagcggcgtgcccagcaggttcagcggcagcggcagcggcaccgacttcaccctgaccatcagcagcctgcagcccgaggacttcgccacctactactgccagcagtactacaactaccccctgaccttcggccagggcaccaaggtggagatcaagagcagc

SEQ ID NO: 895 - protein, 242 aa, synthetic construct
QVQLVQSGAEVKKPGASVKVSCKASGYTFTHYAMDWVRQAPGQGLEWMGGISPQSGGTSYNEKFQGRVTMTRDTSTSTVYMELSSLRSEDTAVYYCARVHGDYHFDYWGQGTLVTVSSGGGGSGGGGSGGGGSDIQMTQSPSSLSASVGDRVTITCQASQDVSTDLAWYQQKPGKAPKLLIYHASSLESGVPSRFSGSGSGTDFTLTISSLQPEDFATYYCQQYYNYPLTFGQGTKVEIKSS

SEQ ID NO: 896 - protein, 5 aa, synthetic construct
AYFMG

SEQ ID NO: 897 - protein, 17 aa, synthetic construct
AINWNGDSSWYRDSVKG

SEQ ID NO: 898 - protein, 16 aa, synthetic construct
DTHGAVGLGSERLYDY

SEQ ID NO: 899 - DNA, 375 bp, synthetic construct
  source 1..375: mol_type = other DNA
caggtgcagctgcaggagagcggcggcggcctggtgcagaccggcgacagcctgaggctgagctgcgtggccagcggcaggaccttcagcgcctacttcatgggctggttcaggcaggcccccggcaaggagagggagttcgtgagcgccatcaactggaacggcgacagcagctggtacagggacagcgtgaagggcaggttcaccgtgagcagggacaacgccaagaacaccgtgtacctgcagatgaacagcctggagcccgaggacaccgccgtgtactactgcgccgccgacacccacggcgccgtgggcctgggcagcgagaggctgtacgactactggggccagggcacccaggtgaccgtgagcagc

SEQ ID NO: 900 - protein, 125 aa, synthetic construct
QVQLQESGGGLVQTGDSLRLSCVASGRTFSAYFMGWFRQAPGKEREFVSAINWNGDSSWYRDSVKGRFTVSRDNAKNTVYLQMNSLEPEDTAVYYCAADTHGAVGLGSERLYDYWGQGTQVTVSS

SEQ ID NO: 901 - protein, 6 aa, synthetic construct
HMNPVT

SEQ ID NO: 902 - protein, 16 aa, synthetic construct
TIDSNDIPDYTDAVKG

SEQ ID NO: 903 - protein, 7 aa, synthetic construct
RIIGRYY

SEQ ID NO: 904 - DNA, 348 bp, synthetic construct
  source 1..348: mol_type = other DNA
gaggtgcaggtgcaggagagcggcggcggcctggtgcaggccggcggcagcctgaccctgacctgcaccaccaccggccccggcgtgctgcacatgaaccccgtgaccaggtacaggcagttccccggccagcagagggagatggtggccaccatcgacagcaacgacatccccgactacaccgacgccgtgaagggcaggttcaccctgagcagggacgccaggaggaccaccgcctacctgaggatgaacaacctgcagagcgacgacaccgccatccacttctgcaacgccaggatcatcggcaggtactactggggccagggcacccaggtgaccgtgagcagc

SEQ ID NO: 905 - protein, 116 aa, synthetic construct
EVQVQESGGGLVQAGGSLTLTCTTTGPGVLHMNPVTRYRQFPGQQREMVATIDSNDIPDYTDAVKGRFTLSRDARRTTAYLRMNNLQSDDTAIHFCNARIIGRYYWGQGTQVTVSS

SEQ ID NO: 906 - protein, 5 aa, synthetic construct
VARMA

SEQ ID NO: 907 - protein, 16 aa, synthetic construct
SITSFGDTNYADSVKG

SEQ ID NO: 908 - protein, 9 aa, synthetic construct
DNNRAFTDY

SEQ ID NO: 909 - DNA, 351 bp, synthetic construct
  source 1..351: mol_type = other DNA
gccgtgcagctggtggacagcggcggcggcctggtgcagcccggcggcagcctgaggctgagctgcgccgccagcggcatcatcttcagcgtggccaggatggcctggtacaggcaggcccccggcaagcagagggagatggtggccagcatcaccagcttcggcgacaccaactacgccgacagcgtgaagggcaggttcaccatcagcaggaacaacgaccagaacaccgtgttcctgcagatggacagcctgaagcccgaggacgccgcccactactactgccacgtggacaacaacagggccttcaccgactactggggccagggcacccaggtgaccgtgagcagc

SEQ ID NO: 910 - protein, 117 aa, synthetic construct
AVQLVDSGGGLVQPGGSLRLSCAASGIIFSVARMAWYRQAPGKQREMVASITSFGDTNYADSVKGRFTISRNNDQNTVFLQMDSLKPEDAAHYYCHVDNNRAFTDYWGQGTQVTVSS

SEQ ID NO: 911 - protein, 11 aa, synthetic construct
RASQSVGNYLA

SEQ ID NO: 912 - protein, 7 aa, synthetic construct
DASTRAT

SEQ ID NO: 913 - protein, 9 aa, synthetic construct
QQSYSYPFT

SEQ ID NO: 914 - protein, 5 aa, synthetic construct
SYAMN

SEQ ID NO: 915 - protein, 17 aa, synthetic construct
WINPGSGNTNYADSVKG

SEQ ID NO: 916 - protein, 10 aa, synthetic construct
YAWDLDAFDY

SEQ ID NO: 917 - DNA, 321 bp, synthetic construct
  source 1..321: mol_type = other DNA
gacatccagatgacccagagccccagcagcctgagcgccagcgtgggcgacagggtgaccatcacctgcagggccagccagagcgtgggcaactacctggcctggtaccagcagaagcccggcaaggcccccaagctgctgatctacgacgccagcaccagggccaccggcgtgcccagcaggttcagcggcagcggcagcggcaccgacttcaccctgaccatcagcagcctgcagcccgaggacttcgccacctactactgccagcagagctacagctaccccttcaccttcggccagggcaccaaggtggagatcaag

SEQ ID NO: 918 - protein, 107 aa, synthetic construct
DIQMTQSPSSLSASVGDRVTITCRASQSVGNYLAWYQQKPGKAPKLLIYDASTRATGVPSRFSGSGSGTDFTLTISSLQPEDFATYYCQQSYSYPFTFGQGTKVEIK

SEQ ID NO: 919 - DNA, 357 bp, synthetic construct
  source 1..357: mol_type = other DNA
gaggtgcagctggtggagagcggcggcggcctggtgcagcccggcggcagcctgaggctgagctgcgccgccagcggcttcaacatcaagagctacgccatgaactgggtgaggcaggcccccggcaagggcctggagtgggtggcctggatcaaccccggcagcggcaacaccaactacgccgacagcgtgaagggcaggttcaccatcagcgccgacaccagcaagaacaccgcctacctgcagatgaacagcctgagggccgaggacaccgccgtgtactactgcgccaggtacgcctgggacctggacgccttcgactactggggccagggcaccctggtgaccgtgagcagc

SEQ ID NO: 920 - protein, 119 aa, synthetic construct
EVQLVESGGGLVQPGGSLRLSCAASGFNIKSYAMNWVRQAPGKGLEWVAWINPGSGNTNYADSVKGRFTISADTSKNTAYLQMNSLRAEDTAVYYCARYAWDLDAFDYWGQGTLVTVSS

SEQ ID NO: 921 - DNA, 723 bp, synthetic construct
  source 1..723: mol_type = other DNA
gacatccagatgacccagagccccagcagcctgagcgccagcgtgggcgacagggtgaccatcacctgcagggccagccagagcgtgggcaactacctggcctggtaccagcagaagcccggcaaggcccccaagctgctgatctacgacgccagcaccagggccaccggcgtgcccagcaggttcagcggcagcggcagcggcaccgacttcaccctgaccatcagcagcctgcagcccgaggacttcgccacctactactgccagcagagctacagctaccccttcaccttcggccagggcaccaaggtggagatcaagggcggcggcggcagcggcggcggcggcagcggcggcggcggcagcgaggtgcagctggtggagagcggcggcggcctggtgcagcccggcggcagcctgaggctgagctgcgccgccagcggcttcaacatcaagagctacgccatgaactgggtgaggcaggcccccggcaagggcctggagtgggtggcctggatcaaccccggcagcggcaacaccaactacgccgacagcgtgaagggcaggttcaccatcagcgccgacaccagcaagaacaccgcctacctgcagatgaacagcctgagggccgaggacaccgccgtgtactactgcgccaggtacgcctgggacctggacgccttcgactactggggccagggcaccctggtgaccgtgagcagc

SEQ ID NO: 922 - protein, 508 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 923 - protein, 467 aa, synthetic construct
DIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKGGGGSGGGGSGGGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 924 - protein, 475 aa, synthetic construct
DIQMTQSPSSLSASVGDRVTITCKSSQSLLYSDNQKNYLAWYQQKPGKAPKLLIYWASTRESGVPSRFSGSGSGTDFTLTISSLQPEDFATYYCQQYYTYPYTFGQGTKLEIKSSGGGGSGGGGSGGGGSEVQLVQSGAEVKKPGASVKVSCKASGYTFTGYHMHWVRQAPGQRLEWMGRINPYNGAASHNQKFKDRVTITRDTSASTAYMELSSLRSEDTAVYYCARGWDYDGGYYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 925 - protein, 473 aa, synthetic construct
DIVMSQSPSSLAVSVGEKVTMSCKSSQSLLYSDNQKNYLAWYQQKPGQSPKLLIYWASTRESGVPDRFTGSGSGTDFTLTISSVKAEDLAVYYCQQYYTYPYTFGGGTKLEIKGGGGSGGGGSGGGGSEVQLQQSGPELVKPGASVKISCKASGYSFTGYHMHWVKQSHVKSLEWIGRINPYNGAASHNQKFKDKATLTVDKSSSTAYMELHSLTSEDSAVYYCARGWDYDGGYYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 926 - protein, 465 aa, synthetic construct
QVQLVQSGAEVKKPGASVKVSCKASGYTFTSYDMSWVRQAPGQGLEWMGAINPSGDFTSYNEKFQGRVTMTRDTSTSTVYMELSSLRSEDTAVYYCARGVGWSFLDYWGQGTLVTVSSGGGGSGGGGSGGGGSDIQMTQSPSSLSASVGDRVTITCQASQDVRTWLAWYQQKPGKAPKLLIYSASSLQTGVPSRFSGSGSGTDFTLTISSLQPEDFATYYCQQSNYLPFTFGQGTKVEIKSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 927 - protein, 465 aa, synthetic construct
QVQLVQSGAEVKKPGASVKVSCKASGYTFTDYAMDWVRQAPGQGLEWMGWISGQSGSTSYNEKFQGRVTMTRDTSTSTVYMELSSLRSEDTAVYYCAKWLDGPWFDVWGQGTLVTVSSGGGGSGGGGSGGGGSDIQMTQSPSSLSASVGDRVTITCKASEDIHSDLAWYQQKPGKAPKLLIYSASSLQSGVPSRFSGSGSGTDFTLTISSLQPEDFATYYCQQAYYLPLTFGQGTKVEIKSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 928 - protein, 465 aa, synthetic construct
QVQLVQSGAEVKKPGASVKVSCKASGYTFTHYAMDWVRQAPGQGLEWMGGISPQSGGTSYNEKFQGRVTMTRDTSTSTVYMELSSLRSEDTAVYYCARVHGDYHFDYWGQGTLVTVSSGGGGSGGGGSGGGGSDIQMTQSPSSLSASVGDRVTITCQASQDVSTDLAWYQQKPGKAPKLLIYHASSLESGVPSRFSGSGSGTDFTLTISSLQPEDFATYYCQQYYNYPLTFGQGTKVEIKSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR